(19) United States
(12) Patent Application Publication
(10) Pub. No.: US 2024/0216357 A1
(43) Pub. Date: Jul. 4, 2024

(54) POLYCYCLIC COMPOUND AND APPLICATION THEREOF

(71) Applicants: SICHUAN HUIYU PHARMACEUTICAL CO., LTD., Neijiang (CN); SICHUAN HUIYU SEACROSS PHARMACEUTICAL TECHNOLOGY CO., LTD., Chengdu (CN)

(72) Inventors: Shoujun CHEN, Neijiang (CN); Jiaming YAN, Neijiang (CN); Zhen WANG, Neijiang (CN); Zhao DING, Neijiang (CN)

(73) Assignees: SICHUAN HUIYU PHARMACEUTICAL CO., LTD., Neijiang (CN); SICHUAN HUIYU SEACROSS PHARMACEUTICAL TECHNOLOGY CO., LTD., Chengdu (CN)

(21) Appl. No.: 18/283,466

(22) PCT Filed: Mar. 24, 2022
(86) PCT No.: PCT/CN2022/082812

§ 371 Date: Sep. 22, 2023

(30) Foreign Application Priority Data
Mar. 24, 2021 (CN) 202110316147.1
Dec. 3, 2021 (CN) 202111470484.2

(51) Int. Cl. A61K 31/46; A61K 31/439; A61K 31/4418; A61K 31/4439; A61K 31/4545; A61K 31/496; A61K 31/4985; A61K 31/5025; A61K 31/506; A61K 31/53; A61K 31/5377; C07D 213/74; C07D 239/28; C07D 239/47; C07D 401/04; C07D 401/12; C07D 401/14; C07D 403/10; C07D 405/14; C07D 413/14; C07D 451/04; C07D 471/04; C07D 487/04; C07D 519/00

(52) U.S. Cl. CPC A61K 31/46; A61K 31/439; A61K 31/4418; A61K 31/4439; A61K 31/4545; A61K 31/496; A61K 31/4985; A61K 31/5025; A61K 31/506; A61K 31/53; A61K 31/5377; C07D 213/74; C07D 239/28; C07D 239/47; C07D 401/04; C07D 401/12; C07D 401/14; C07D 403/10; C07D 405/14; C07D 413/14; C07D 451/04; C07D 471/04; C07D 487/04; C07D 519/00

(57) ABSTRACT

Provided are a compound represented by general formula (I), or a tautomer, mesomer, racemate, enantiomer, diastereomer or mixture thereof, a deuterium isotopic derivative, pharmaceutically acceptable hydrate, solvate, salt, or eutectic thereof, a pharmaceutical composition thereof, and an application thereof in preparing a medicament for lysine specific demethylase 1 inhibitor-related diseases.

### CROSS REFERENCE TO THE RELATED APPLICATIONS

This application is the national phase entry of International Application No. PCT/CN2022/082812, filed on Mar. 24, 2022, which is based upon and claims priority to Chinese Patent Applications No. 202110316147.1, filed on Mar. 24, 2021, and No. 202111470484.2, filed on Dec. 3, 2021, the entire contents of which are incorporated herein by reference.

### TECHNICAL FIELD

The present invention relates to the field of medicinal chemistry, and specifically relates to a compound as lysine-specific demethylase 1 (LSD1) inhibitors, and a use thereof in the preparation of a medicament for the treatment of LSD1-related diseases.

### BACKGROUND

LSD1 protein (Lysine Specific Demethylase 1, histone demethylase, also known as KDM1A), consists of 852 amino acids and has a molecular weight of 93 kDa. It was first discovered and reported in 2004 by Harvard University's Shi Yang team (Shi, Y., Lan, F., Matson, C., Mulligan, P., Whetstine, J. R., Cole, P. A., Casero, R. A., and Shi, Y Histone demethylation mediated by the nuclear amine oxidase homolog LSD1. Cell 2004, 119, 941-953). Studies have confirmed that LSD1 exerts its biological function not only by histone demethylation but also by demethylation of non-histone proteins p53 and Dnmtl. The biological role of LSD1 is mainly manifested in the regulation of sex hormone receptor-mediated gene transcription; the regulation of tumor cell proliferation, apoptosis and metastasis; and the regulation of embryonic development (Ancelin, K.; Syx, L.; et al., eLife 2016, 5, e08851/1-e08851/24.), mitosis and other processes. Moreover, LSD1 has also been reported to be associated with osteoporosis (Sun, J.; Ermann, J.; et al., Bone Res. 2018, 6(1), 1-12). In addition, LSD1 inhibition studies have found that it is related to macrophage type polarization (Tan, A. H. Y; Tu, W. J.; et al., Front. Immunol. 2019, 10, 1351.) and CD8+ T cell infiltration in a tumor microenvironment (Hatzi, K.; Geng, H.; et al., Nature Immunology 2019, 20(1), 86-96). LSD1 is widely expressed in the body, among which the liver, pancreas and salivary glands secrete less LSD1, while testicular tissues express more LSD1 at a level similar to that of other tissues. Studies have found that LSD1 expression levels are significantly increased in different tumor tissues, such as neuroblastoma; breast cancer (Wang, Y; Zhang, H.; et al., Cell 2009, 138(4), 660-72.); prostate cancer (Zhao, L.-J.; Fan, Q.-Q.; et al., Pharmacol. Res. 2020, 159, 104991); pancreatic cancer (Sehrawat, A.; Gao, L.; et al., Proc. Nat. Acad. Sci. USA 2018, 115(18), E4179-E4188.); colon cancer and glioma and blood tumors (Hatzi, K.; Geng, H.; et al., Nature Immunology 2019, 20(1), 86-96.), and high expression of LSD1 is often associated with poorer tumor prognosis and recurrence after treatment (Lynch, J.; Harris, W.; et al., Expert Opinion on Therapeutic Targets 2012, 16(12), 1239-1249.). For a human cancer public database, the researchers also found that LSD1 in some types of cancer showed a trend of overexpression. The survival time of patients with high LSD1 expression was significantly shortened, suggesting that LSD1 overexpression was a poor prognostic factor. In addition, it was also found that LSD1 was highly expressed in a variety of cancer tissues, and more and more reports indicated that LSD1 was involved in a variety of tumor processes and embryonic development in an epigenetic regulator. A TCGA cancer database also showed that LSD1 expression showed a negative correlated with IFN antiviral effects and CD8 T cell infiltration, which was also consistent with test results in a mouse model. Therefore, the inhibition of LSD1 can enhance the immunogenicity of tumors and promote T cell infiltration, activate anti-tumor T cell immunity, and can be used as a target for tumor treatment with anti-PD-1 immunotherapy. The function of inhibiting LSD1 can enhance the expression of endogenous retroviral elements (ERVs) and inhibit the function of RNA-induced silencing complex (RISC), resulting in overexpression of double-stranded RNA (dsRNA) and activation of type I interferon (IFN) (Doll, S., Kriegmair, M. C., Santos, A., Wierer, M., Coscia, F., Neil, H. M., et al. Rapid proteomic analysis for solid tumors reveals LSD1 as a drug target in an end-stage cancer patient. Molecular Oncology, 2018, 12(8), 1296-1307.). The research results also suggest that inhibition of DNA methylation either alone or in combination with HDAC inhibitors can lead to activation of an tumor interferon (IFN) pathway and enhance the efficacy of immunotherapy in tumors. At the same time, blocking DNA methylation in T cells can also enhance PD-1/PD-L1 immunotherapy-mediated T cell activity and tumor suppression (Chiappinelli, K. B., Strissel, P. L., Desrichard, A., Li, H., Henke, C., Akman, B., Hein, A., Rote, N. S., Cope, L. M., Snyder, A., et al. Inhibiting DNA methylation causes an interferon response in cancer via dsRNA including endogenous retroviruses. Cell 2015, 162, 974-986; Topper, M. J., Vaz, M., Chiappinelli, K. B., DeStefano Shields, C. E., Niknafs, N., Yen, R. C., Wenzel, A., Hicks, J., Ballew, M., Stone, M., et al. Epigenetic therapy ties MYC depletion to reversing immune evasion and treating lung cancer. Cell 2017, 171, 1284-130; Ghoneim, H. E., Fan, Y, Moustaki, A., Abdelsamed, H. A., Dash, P., Dogra, P., Carter, R., Awad, W., Neale, G., Thomas, P. G., et al. De novo epigenetic programs inhibit PD-1 blockade-mediated t cell rejuvenation. Cell 2017, 170, 142-157). Therefore, the development of LSD1 inhibitors is a hot spots in the field of oncology research.

At present, there are still no drugs on the market worldwide for targeting LSD1, and compounds under study are in the early clinical or preclinical research stage. Although many companies or research institutions have conducted corresponding research on LSD1 inhibitors and reported relevant patents, for example, GSK has reported a class of cyclopropylamine compounds as LSD1 inhibitors for the treatment of cancers (Neil W. Johnson et al., U.S. Pat. No. 10,064,854; DECAPRIO, J. A. et al., WO2019075327). Celgene reports a class of substituted heterocyclic LSD1 inhibitors for the treatment of cancers (Y. K. Chen, et al., US20180325900). However, some of the reported LSD1 inhibitors have a narrow therapeutic window due to their poor activity, and some others are not very satisfactory, so there is still a great need to develop a new LSD1 inhibitor in this field, especially an LSD1 inhibitor with high activity and superior druggability.

### SUMMARY

The present invention provides a compound of formula (I), or a tautomer, mesomer, racemate, enantiomer, diastereoisomer, or mixture thereof, or a deuterated isotopic derivative, pharmaceutically acceptable hydrate, solvate, salt or cocrystal thereof,

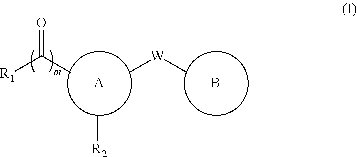

wherein
ring A is selected from C₆₋₁₀ aryl, C₃₋₁₀ heteroaryl, C₃₋₁₀ cycloalkyl, C₂₋₁₀ heterocycloalkyl, C₃₋₁₀ cycloalkenyl, C₂₋₁₀ heterocycloalkenyl, C₆₋₁₀ aryl fused C₃₋₁₀ cycloalkyl, C₆₋₁₀ aryl fused C₂₋₁₀ heterocycloalkyl, C₆₋₁₀ aryl fused C₃₋₁₀ cycloalkenyl, C₆₋₁₀ aryl fused C₂₋₁₀ heterocycloalkenyl, 5-10-membered heteroaryl fused C₃₋₁₀ cycloalkyl, 5-10-membered heteroaryl fused C₂₋₁₀ heterocycloalkyl, 5-10-membered heteroaryl fused C₃₋₁₀ cycloalkenyl, and 5-10-membered heteroaryl fused C₂₋₁₀ heterocycloalkenyl; the above groups are optionally substituted by 0 to 4 substituents selected from halogen, —CN, hydroxy, C₁₋₆ alkyl, halogen substituted C₁₋₆ alkyl, C₁₋₆ alkoxy, halogen substituted C₁₋₆ alkoxy, C₆₋₁₀ aryl substituted C₁₋₆ alkoxy, —OC₃₋₁₀ cycloalkyl, C₃₋₁₀ cycloalkyl, —O—C₁₋₆ alkyl-(C₃₋₁₀ cycloalkyl), —O—(C₂₋₁₀ heterocycloalkyl), C₂₋₁₀ heterocycloalkyl, —O—C₁₋₆ alkyl-(C₂₋₁₀ heterocycloalkyl), C₂₋₆ alkenyl, C₂₋₆ alkynyl, ═O, —NR_{c}R_{d}, and

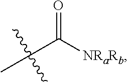

and the ═O can only be a substituent of a non-aromatic ring; and the heteroaryl, heterocycloalkyl, heterocycloalkenyl, C₆₋₁₀ aryl fused C₂₋₁₀ heterocycloalkyl, C₆₋₁₀ aryl C₂₋₁₀ fused heterocycloalkenyl, 5-10-membered heteroaryl fused C₃₋₁₀ cycloalkyl, 5-10-membered heteroaryl fused C₂₋₁₀ heterocycloalkyl, 5-10-membered heteroaryl fused C₃₋₁₀ cycloalkenyl, 5-10-membered heteroaryl fused C₂₋₁₀ heterocycloalkenyl each contain 1 to 4 heteroatoms selected from N, O or S.

Preferably, ring A is selected from C₆₋₁₀ aryl, 5-10-membered heteroaryl, C₃₋₁₀ cycloalkyl, C₂₋₁₀ heterocycloalkyl, C₃₋₁₀ cycloalkenyl, C₂₋₁₀ heterocycloalkenyl, C₆₋₁₀ aryl fused C₃₋₁₀ cycloalkyl, C₆₋₁₀ aryl fused C₂₋₁₀ heterocycloalkyl, C₆₋₁₀ aryl fused C₃₋₁₀ cycloalkenyl, C₆₋₁₀ aryl fused C₂₋₁₀ heterocycloalkenyl, 5-10-membered heteroaryl fused C₃₋₁₀ cycloalkyl, 5-10-membered heteroaryl fused C₂₋₁₀ heterocycloalkyl, 5-10-membered heteroaryl fused C₃₋₁₀ cycloalkenyl, and 5-10-membered heteroaryl fused C₂₋₁₀ heterocycloalkenyl; the above groups are optionally substituted by 0 to 4 substituents selected from halogen, —CN, hydroxy, C₁₋₆ alkyl, halogen substituted C₁₋₆ alkyl, C₁₋₆ alkoxy, halogen substituted C₁₋₆ alkoxy, C₆₋₁₀ aryl substituted C₁₋₆ alkoxy, —OC₃₋₁₀ cycloalkyl, C₃₋₁₀ cycloalkyl, C₂₋₆ alkenyl, C₂₋₆ alkynyl, ═O, —NR_{c}R_{d}, and

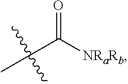

and the ═O can only be a substituent of a non-aromatic ring; and the heteroaryl, heterocycloalkyl, heterocycloalkenyl, C₆₋₁₀ aryl fused C₂₋₁₀ heterocycloalkyl, C₆₋₁₀ aryl fused C₂₋₁₀ heterocycloalkenyl, 5-10-membered heteroaryl fused C₃₋₁₀ cycloalkyl, 5-10-membered heteroaryl fused C₂₋₁₀ heterocycloalkyl, 5-10-membered heteroaryl fused C₃₋₁₀ cycloalkenyl, 5-10-membered heteroaryl fused C₂₋₁₀ heterocycloalkenyl each contain 1 to 4 heteroatoms selected from N, O or S.

Preferably, ring A is selected from C₆₋₁₀ aryl, C₃₋₁₀ heteroaryl, C₃₋₁₀ cycloalkyl, and C₃₋₁₀ heterocycloalkyl; the aryl, the heteroaryl, the cycloalkyl and the heterocycloalkyl are optionally substituted by 0 to 4 substituents selected from halogen, CN, CF₃, hydroxy, C₁₋₆ alkyl, C₁₋₆ alkoxy, C₂₋₆ alkenyl, C₂₋₆ alkynyl, ═O, or

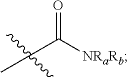

the heteroaryl and the heterocycloalkyl each contain 1 to 4 heteroatoms selected from N, O or S;

ring B is selected from C₆₋₁₀ aryl, C₃₋₁₀ heteroaryl, C₃₋₁₀ cycloalkyl, and C₂₋₁₀ heterocycloalkyl; the aryl, the heteroaryl, the cycloalkyl and the heterocycloalkyl are optionally substituted by 0 to 4 substituents selected from halogen, —CN, hydroxy, C₁₋₆ alkyl, halogen substituted C₁₋₆ alkoxy, C₃₋₁₀ cycloalkyl, C₂₋₁₀ heterocycloalkyl, C₂₋₆ alkenyl, C₂₋₆ alkynyl, or —NR_{c}R_{d}; the heteroaryl and the heterocycloalkyl each contain 1 to 4 heteroatoms selected from N, O or S; provided that ring B is not a 9-membered spiro ring.

Preferably, ring B is selected from C₆₋₁₀ aryl, 5-10-membered heteroaryl, C₃₋₁₀ cycloalkyl, and C₂₋₁₀ heterocycloalkyl; the aryl, the heteroaryl, the cycloalkyl and the heterocycloalkyl are optionally substituted by 0 to 4 substituents selected from halogen, —CN, CF₃, hydroxy, C₁₋₆ alkyl, halogen substituted C₁₋₆ alkyl, C₁₋₆ alkoxy, halogen substituted C₁₋₆ alkoxy, C₃₋₁₀ cycloalkyl, C₂₋₁₀ heterocycloalkyl, C₂₋₆ alkenyl, C₂₋₆ alkynyl, or —NR_{c}R_{d}; the heteroaryl and the heterocycloalkyl each contain 1 to 4 heteroatoms selected from N, O or S; provided that ring B is not a 9-membered spiro ring.

Preferably, ring B is selected from C₆₋₁₀ aryl, C₃₋₁₀ heteroaryl, C₃₋₁₀ cycloalkyl, and C₃₋₁₀ heterocycloalkyl; the aryl, the heteroaryl, the cycloalkyl and the heterocycloalkyl are optionally substituted by 0 to 4 substituents selected from halogen, —CN, CF₃, hydroxy, C₁₋₆ alkyl, C₁₋₆ alkoxy, C₂₋₆ alkenyl, C₂₋₆ alkynyl, or —NR_{c}R_{d}; the heteroaryl and the heterocycloalkyl each contain 1 to 4 heteroatoms selected from N, O or S; provided that ring B is not a 9-membered spiro ring.

W is selected from a bond,

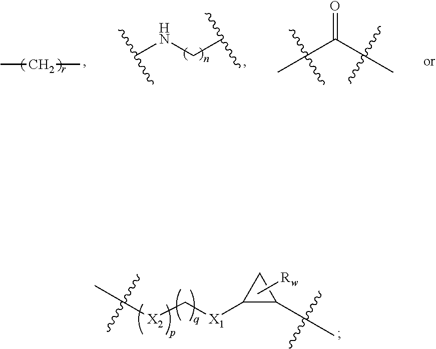

and when W is a bond, ring A and ring B are directly connected via the bond.

Preferably, W is selected from a bond, —CH₂—,

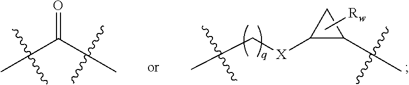

and when W is a bond, ring A and ring B are directly connected via the bond.

X₁ is selected from —NRₓ—, —O—, or —CHRₓ—.

X₂ is selected from —NH—, or —O—.

R_{W} and Rₓ are independently selected from H, C₁₋₆ alkyl, C₁₋₆ alkoxy, halogen, —CN, C₂₋₆ alkenyl, C₂₋₆ alkynyl, —C(═O)C₁₋₆ alkyl, —OC₃₋₆ cycloalkyl, C₆₋₁₀ aryl, C₅₋₁₀ heteroaryl, C₃₋₁₀ cycloalkyl, and C₂₋₁₀ heterocycloalkyl; the alkyl, the alkenyl, the alkynyl, the aryl, the heteroaryl, the cycloalkyl or heterocycloalkyl are optionally substituted by 0 to 4 substituents selected from halogen, halogen substituted C₁₋₆ alkyl, —CN, hydroxy, C₁₋₆ alkyl, C₁₋₆ alkoxy, —C(═O)C₁₋₆ alkyl, —OC₃₋₆ cycloalkyl or C₁₋₄ alkylthio; the heterocyclyl contains 1 to 3 heteroatoms selected from N, O or S.

R₁ and R₂ are independently selected from hydrogen, C₁₋₆ alkyl, C₁₋₆ alkoxy, halogen, —CN, C₂₋₆ alkenyl, C2-6 alkynyl, —C(═O)C₁₋₆ alkyl, —OC₃₋₆ cycloalkyl, —OC₆₋₁₀ aryl, —O—(5-10-membered heteroaryl), C₆₋₁₀ aryl, C₅₋₁₀ heteroaryl, C₃₋₁₀ cycloalkyl, C₂₋₁₀ heterocycloalkyl, C₃₋₁₀ cycloalkenyl, C₂₋₁₀ heterocycloalkenyl, C₆₋₁₀ aryl fused C₂₋₁₀ heterocycloalkyl, and —NR₄R₅; the alkyl, the alkenyl, the alkynyl, the aryl, the heteroaryl, the cycloalkyl, the heterocycloalkyl, the cycloalkenyl, and the heterocycloalkenyl are optionally further substituted by 0 to 4 substituents selected from halogen, —CN, —CH₂CN, —NH₂, hydroxy, C₁₋₆ alkyl, halogen substituted C₁₋₆ alkyl, hydroxy substituted C₁₋₆ alkyl, C₁₋₆ alkoxy, halogen substituted C₁₋₆ alkoxy, hydroxy substituted C₁₋₆ alkoxy, ═O, —C(═O)C₁₋₆ alkyl, —C(═O)OC₁₋₆ alkyl, —COOH, C₂₋₆ alkynyl, hydroxy substituted C₂₋₆ alkynyl, —OC₃₋₆ cycloalkyl, C₁₋₄ alkylthio, C₅₋₁₀ heteroaryl, C₆₋₁₀ aryl, C₄₋₁₀ cycloalkyl, C₂₋₁₀ heterocycloalkyl, and —S(O)₂Rₐ; the heteroaryl and heterocycloalkyl each contain 1 to 3 heteroatoms selected from N, O or S.

Preferably, R₁ and R₂ are independently selected from hydrogen, C₁₋₆ alkyl, C₁₋₆ alkoxy, halogen, —CN, C₂₋₆ alkenyl, C2-6 alkynyl, —C(═O)C₁₋₆ alkyl, —OC₃₋₆ cycloalkyl, —OC₆₋₁₀ aryl, —O—(5-10-membered heteroaryl), C₆₋₁₀ aryl, 5-10-membered heteroaryl, C₃₋₁₀ cycloalkyl, C₂₋₁₀ heterocycloalkyl, C₆₋₁₀ aryl fused C₂₋₁₀ heterocycloalkyl, and —NR₄R₅; the alkyl, the alkenyl, the alkynyl, the aryl, the heteroaryl, the cycloalkyl, or the heterocycloalkyl are optionally further substituted by 0 to 4 substituents selected from halogen, —CN, —CH₂CN, —NH₂, hydroxy, C₁₋₆ alkyl, halogen substituted C₁₋₆ alkyl, hydroxy substituted C₁₋₆ alkyl, C₁₋₆ alkoxy, halogen substituted C₁₋₆ alkoxy, hydroxy substituted C₁₋₆ alkoxy, —C(═O)C₁₋₆ alkyl, —C(═O)OC₁₋₆ alkyl, —COOH, C₂₋₆ alkynyl, hydroxy substituted C₂₋₆ alkynyl, —OC₃₋₆ cycloalkyl, C₁₋₄ alkylthio, 5-10-membered heteroaryl, C₆₋₁₀ aryl, C₄₋₁₀ cycloalkyl, C₂₋₁₀ heterocycloalkyl, and —S(O)₂R₆; the heteroaryl and heterocycloalkyl each contain 1 to 3 heteroatoms selected from N, O or S.

Preferably, R₁ and R₂ are independently selected from hydrogen, C₁₋₆ alkyl, C₁₋₆ alkoxy, halogen, —CN, C₂₋₆ alkenyl, C₂₋₆ alkynyl, —C(═O)C₁₋₆ alkyl, —(═O)C₁₋₆ alkyl, —OC₃₋₆ cycloalkyl, C₄₋₁₀ cycloalkyl, C₆₋₁₀ aryl, C₅₋₁₀ heteroaryl, C₃₋₁₀ cycloalkyl, and C₃₋₁₀ heterocycloalkyl; the alkyl, the alkenyl, the alkynyl, the aryl, the heteroaryl, the cycloalkyl, or the heterocycloalkyl is optionally further substituted by 0 to 4 substituents selected from halogen, CF₃, —CN, hydroxy, C₁₋₆ alkyl, C₁₋₆ alkoxy, —C(═O)C₁₋₆ alkyl, —(═O)C₁₋₆ alkyl, —OC₃₋₆ cycloalkyl, C₁₋₄ alkylthio, C₅₋₁₀ heteroaryl, C₆₋₁₀ aryl, C₄₋₁₀ cycloalkyl, C₃₋₁₀ heterocycloalkyl, and —S(O)₂Rₐ; the heterocyclyl contains 1 to 3 heteroatoms selected from N, O or S.

R₄ and R₅ are independently selected from hydrogen, C₁₋₆ alkyl, amino substituted C₁₋₆ alkyl, C₃₋₁₀ cycloalkyl, C₂₋₁₀ heteroaryl, C₆₋₁₀ aryl, and 5-10-membered heteroaryl; and R₆ is selected from C₁₋₆ alkyl, and amino.

Rₐ and R_{b} are independently selected from hydrogen, C₁₋₆ alkyl, C₁₋₆ alkoxy, halogen, —CN, C₂₋₆ alkenyl, C₂₋₆ alkynyl, —C(═O)C₁₋₆ alkyl, —OC₃₋₆ cycloalkyl, C₄₋₁₀ cycloalkyl, C₆₋₁₀ aryl, C₅₋₁₀ heteroaryl, C₃₋₁₀ cycloalkyl, and C₂₋₁₀ heterocycloalkyl; the alkyl, the alkenyl, the alkynyl, the aryl, the heteroaryl, the cycloalkyl, or the heterocycloalkyl is optionally further substituted by 0 to 4 substituents selected from halogen, halogen substituted C₁₋₆ alkyl, —CN, hydroxy, C₁₋₆ alkyl, C₁₋₆ alkoxy, —C(═O)C₁₋₆ alkyl, —OC₃₋₆ cycloalkyl, C₁₋₄ alkylthio, C₅₋₁₀ heteroaryl, C₆₋₁₀ aryl, C₄₋₁₀ cycloalkyl, C₂₋₁₀ heterocycloalkyl, and —S(O)₂Rₐ; and the heterocyclyl contains 1 to 3 heteroatoms selected from N, O or S.

Rₐ and R_{b} are independently selected from hydrogen, C₁₋₆ alkyl, C₁₋₆ alkoxy, halogen, —CN, C₂₋₆ alkenyl, C₂₋₆ alkynyl, —C(═O)C₁₋₆ alkyl, —OC₃₋₆ cycloalkyl, C₄₋₁₀ cycloalkyl, C₆₋₁₀ aryl, C₅₋₁₀ heteroaryl, C₃₋₁₀ cycloalkyl, and C₂₋₁₀ heterocycloalkyl; the alkyl, the alkenyl, the alkynyl, the aryl, the heteroaryl, the cycloalkyl, or the heterocycloalkyl is optionally further substituted by 0 to 4 substituents selected from halogen, halogen substituted C₁₋₆ alkyl, —CN, hydroxy, C₁₋₆ alkyl, C₁₋₆ alkoxy, —C(═O)C₁₋₆ alkyl, —OC₃₋₆ cycloalkyl, C₁₋₄ alkylthio, C₅₋₁₀ heteroaryl, C₆₋₁₀ aryl, C₄₋₁₀ cycloalkyl, C₃₋₁₀ heterocycloalkyl, and —S(O)₂R₆; the heterocyclyl contains 1 to 3 heteroatoms selected from N, O or S; R₆ is selected from C₁₋₆ alkyl, and amino.

Rₐ and R_{b} are independently selected from hydrogen, C₁₋₆ alkyl, C₁₋₆ alkoxy, halogen, —CN, C₂₋₆ alkenyl, C₂₋₆ alkynyl, —C(═O)C₁₋₆ alkyl, —(═O)C₁₋₆ alkyl, —OC₃₋₆ cycloalkyl, C₄₋₁₀ cycloalkyl, C₆₋₁₀ aryl, C₅₋₁₀ heteroaryl, C₃₋₁₀ cycloalkyl, and C₃₋₁₀ heterocycloalkyl; the alkyl, the alkenyl, the alkynyl, the aryl, the heteroaryl, the cycloalkyl, or the heterocycloalkyl is optionally further substituted by 0 to 4 substituents selected from halogen, CF₃, CN, hydroxy, C₁₋₆ alkyl, C₁₋₆ alkoxy, —C(═O)C₁₋₆ alkyl, —(═O)C₁₋₆ alkyl, —OC₃₋₆ cycloalkyl, C₁₋₄ alkylthio, C₅₋₁₀ heteroaryl, C₆₋₁₀ aryl, C₄₋₁₀ cycloalkyl, C₃₋₁₀ heterocycloalkyl, and —S(O)₂Rₐ; the heterocyclyl contains 1 to 3 heteroatoms selected from N, O or S.

R_{c} and R_{d} are independently selected from hydrogen, C₁₋₆ alkyl, C₁₋₆ alkoxy, halogen, —CN, C₂₋₆ alkenyl, C₂₋₆ alkynyl, —C(═O)C₁₋₆ alkyl, —OC₃₋₆ cycloalkyl, C₃₋₁₀ cycloalkyl, C₆₋₁₀ aryl, C₅₋₁₀ heteroaryl, C₄₋₁₀ cycloalkyl, and C₂₋₁₀ heterocycloalkyl; the alkyl, the alkenyl, the alkynyl, the aryl, the heteroaryl, the cycloalkyl, or the heterocycloalkyl is optionally further substituted by 0 to 4 substituents selected from halogen, hydroxy, amino, aminoalkyl, aminocycloalkyl, aminoheterocyclyl, C₁₋₆ alkyl, C₁₋₆ alkoxy, C₅₋₁₀ heteroaryl, aryl, hydroxy, —CN, and —S(O)₂Rₐ; the heterocyclyl contains 1 to 3 heteroatoms selected from N, O or S; R₆ is selected from C₁₋₆ alkyl, and amino; when ring A is selected from a aromatic heterocycle fused by a 5-membered ring and a 6-membered ring, R_{c} and R_{d} are not simultaneously methyl.

R_{c} and R_{d} are independently selected from hydrogen, C₁₋₆ alkyl, C₁₋₆ alkoxy, halogen, —CN, C₂₋₆ alkenyl, C₂₋₆ alkynyl, —C(═O)C₁₋₆ alkyl, —OC₃₋₆ cycloalkyl, C₃₋₁₀ cycloalkyl, C₆₋₁₀ aryl, C₅₋₁₀ heteroaryl, C₄₋₁₀ cycloalkyl, and C₂₋₁₀ heterocycloalkyl; the alkyl, the alkenyl, the alkynyl, the aryl, the heteroaryl, the cycloalkyl, or the heterocycloalkyl is optionally further substituted by 0 to 4 substituents selected from halogen, hydroxy, amino, aminoalkyl, aminocycloalkyl, aminoheterocyclyl, C₁₋₆ alkyl, C₁₋₆ alkoxy, C₅₋₁₀ heteroaryl, aryl, hydroxy, —CN, and —S(O)₂R₆; the heterocyclyl contains 1 to 3 heteroatoms selected from N, O or S; R₆ is selected from C₁₋₆ alkyl, and amino; when ring A is selected from a aromatic heterocycle fused by a 5-membered ring and a 6-membered ring, R_{c} and R_{d} are not simultaneously methyl.

R_{c} and R_{d} are independently selected from hydrogen, C₁₋₆ alkyl, C₁₋₆ alkoxy, halogen, —CN, C₂₋₆ alkenyl, C₂₋₆ alkynyl, —C(═O)C₁₋₆ alkyl, —(═O)C₁₋₆ alkyl, —OC₃₋₆ cycloalkyl, C₃₋₁₀ cycloalkyl, C₆₋₁₀ aryl, C₅₋₁₀ heteroaryl, C₄₋₁₀ cycloalkyl, and C₃₋₁₀ heterocycloalkyl; the alkyl, the alkenyl, the alkynyl, the aryl, the heteroaryl, the cycloalkyl, or the heterocycloalkyl is optionally further substituted by 0 to 4 substituents selected from halogen, amino, aminoalkyl, aminocycloalkyl, aminoheterocyclyl, C₁₋₆ alkyl, C₁₋₆ alkoxy, C₅₋₁₀ heteroaryl, aryl, hydroxy, CN, and —S(O)₂Rₐ; the heterocyclyl contains 1 to 3 heteroatoms selected from N, O or S; when ring A is selected from a aromatic heterocycle fused by a 5-membered ring and a 6-membered ring, R_{c} and R_{d} are not simultaneously methyl; m and n are independently selected from integers from 0 to 6; when m is 0, R₁ is directly connected to ring A.

Preferably, m and n are each independently selected from 0, 1, 2, 3, 4 or 5; when m is selected from 0, R₁ is directly connected to ring A.

Preferably, m is selected from 0 or 1; and when m is 0, R₁ is directly connected to ring A.

Preferably, m is selected from 0.

Preferably, n is an integer selected from 0 to 6; when n is selected from 0,

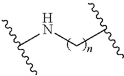

means —NH—.

r is an integer selected from 1 to 6.

p is selected from 0 or 1; and when p is 0, X₂ is absent.

q is selected from 1, 2, 3 or 4.

The present invention discloses a compound of formula (I), wherein ring A of formula (I) is selected from:

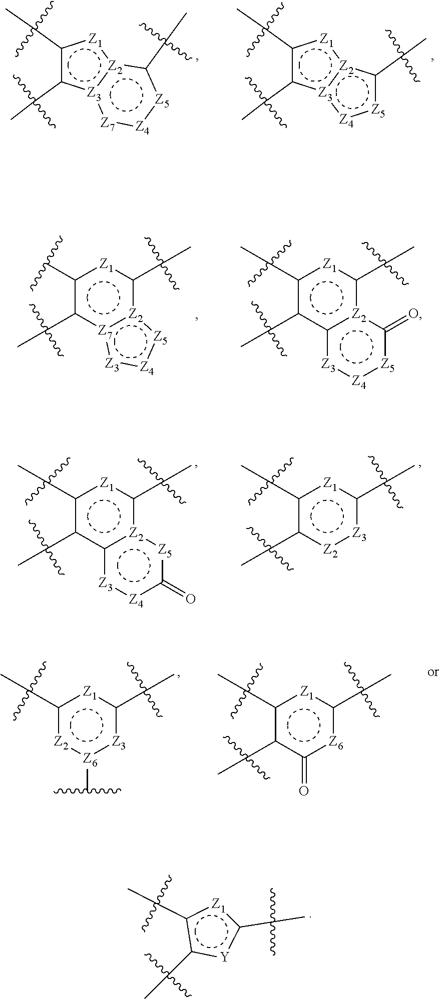

Preferably, ring A is selected from

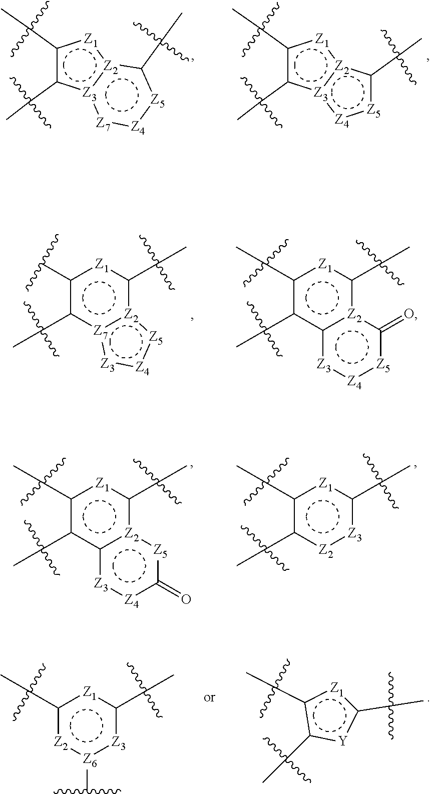

Preferably, ring A is selected from

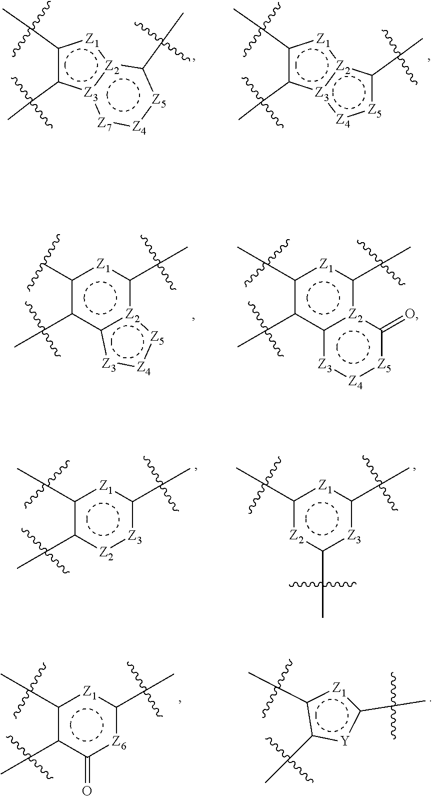

Z₁, Z₂, Z₃, Z₄, Z₅, Z₆, and Z₇ are each independently selected from O, S, N, —NR₃—,

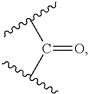 [SMILES: *C(*)=O]

### ═CR_{z}—,

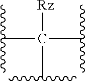

or —CR_{y}R_{z}—.

R₃ is selected from hydrogen or C₁₋₆ alkyl.

Preferably, Z₁, Z₂, Z₃, Z₄, and Z₅ are each independently selected from N, —NH— or —CR_{z}—.

Preferably, Z₆ is selected from —NH— or CR_{z}.

R_{y} and R_{z} are independently selected from hydrogen, halogen, —CN, hydroxy, C₁₋₆ alkyl, halogen substituted C₁₋₆ alkyl, C₁₋₆ alkoxy, halogen substituted C₁₋₆ alkoxy, C₆₋₁₀ aryl substituted C₁₋₆ alkoxy, —OC₃₋₁₀ cycloalkyl, C₃₋₁₀ cycloalkyl, —O—C₁₋₆ alkyl-(C₃₋₁₀ cycloalkyl), —O—(C₂₋₁₀ heterocycloalkyl), C₂₋₁₀ heterocycloalkyl, —O—C₁₋₆ alkyl-(C₂₋₁₀ heterocycloalkyl), C₂₋₆ alkenyl, C₂₋₆ alkynyl, —NR_{c}R_{d}, and

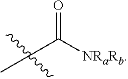

Preferably, R_{y} and R_{z} are independently selected from hydrogen, halogen, —CN, hydroxy, C₁₋₆ alkyl, halogen substituted C₁₋₆ alkyl, C₁₋₆ alkoxy, halogen substituted C₁₋₆ alkoxy, C₆₋₁₀ aryl substituted C₁₋₆ alkoxy, —OC₃₋₁₀ cycloalkyl, C₃₋₁₀ cycloalkyl, C₂₋₆ alkenyl, C₂₋₆ alkynyl, —NR_{c}R_{d},

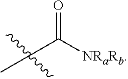

Preferably, R_{y} and R_{z} are each independently selected from hydrogen, —CN, hydroxy, C₁₋₆ alkyl, C₁₋₆ alkoxy, halogen substituted C₁₋₆ alkoxy, C₆₋₁₀ aryl substituted methoxy, —OC₃₋₁₀ cycloalkyl, and

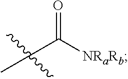

Rₐ and R_{b} are both hydrogen.

Further preferably, R_{y} and R_{z} are each independently selected from hydrogen, —CN, hydroxy, methyl, methoxy, ethoxy, propoxy, difluoromethoxy, benzyloxy, cyclopentaneoxy, and —C(O)NH₂.

Preferably, R_{z} is selected from hydrogen, halogen, CN, hdroxy, C₁₋₆ alkyl, C₁₋₆ alkoxy, C₃₋₁₀ cycloalkyl, C₂₋₆ alkenyl, C₂₋₆ alkynyl, ═O, NRₑR_{d} or

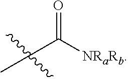

Y is selected from O or S.

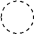

Represents a double bond or absence that may exist anywhere within the ring.

Preferably,

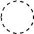

represents an aromatic ring.

The present disclosure provides a compound of formula (I-1),

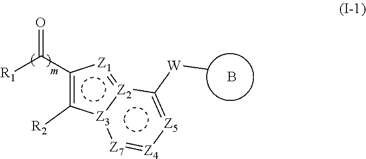

wherein the substituents of formula (I-1) are as defined above.

The present disclosure provides a compound of formula (I-1a),

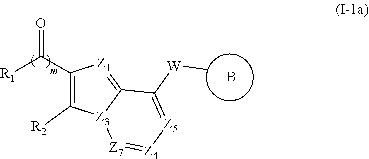

wherein Z₁, Z₃, Z₄, Z₅, and Z₇ are each independently selected from N or =CR_{z}—; and W is a bond.

In an aspect of the present disclosure, wherein Z₃ is N; and Z₁, Z₄, Z₅, and Z₇ are each independently selected from N or ═CR_{z}—.

In an aspect of the present disclosure, wherein ring A is selected from

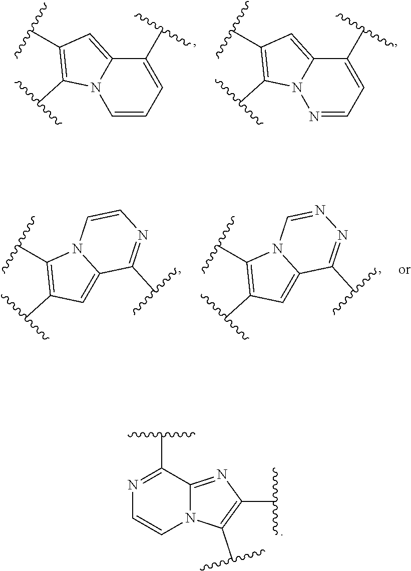 [SMILES: *c1cc2c(*)cccn2c1* ; *c1cc2c(*)ccnn2c1* ; *c1cc2c(*)nccn2c1* ; *c1cc2c(*)nncn2c1* ; *c1nc2c(*)nccn2c1*]

In an aspect of the present disclosure, a compound of formula (I-1b) is provided,

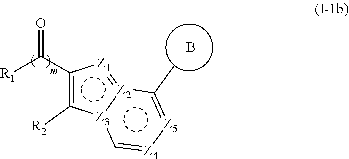

wherein the substituents are as defined above.

In an aspect of the present disclosure, wherein

ring B is selected from C₆₋₁₀ aryl, C₃₋₁₀ heteroaryl, and C₃₋₁₀ heterocycloalkyl; the aryl, the heteroaryl, and the heterocycloalkyl are optionally substituted by 0 to 4 substituents selected from F, CN, CF₃, hydroxy, C₁₋₆ alkyl, C₁₋₆ alkoxy or NR_{c}R_{d}; the heteroaryl and the heterocycloalkyl each contain 1 to 4 heteroatoms selected from N, O or S.

R_{c} and R_{d} are independently selected from hydrogen, F, CN, hydroxy, C₁₋₆ alkyl, or C₁₋₆ alkoxy.

Z₁, Z₂, Z₃, Z₄, and Z₅ are each independently selected from N or —CR_{z}—.

R_{z} is selected from hydrogen, C₁₋₆ alkyl, or C₃₋₁₀ cycloalkyl.

R₁ and R₂ are independently selected from C₅₋₁₀ heteroaryl and C₆₋₁₀ aryl; the heteroaryl and the aryl are each optionally substituted by 0 to 4 substituents selected fromF, amino, aminoalkyl, aminocycloalkyl, aminoheterocyclyl, C₁₋₆ alkyl, C₁₋₆ alkoxy, C₅₋₁₀ heteroaryl, aryl, hydroxy, and CN; the heteroaryl contains 1 to 3 heteroatoms selected from N, O or S. m is selected from 0, or 1.

Preferably, ring B is selected from C₆₋₁₀ aryl, C₃₋₁₀ heteroaryl, and C₃₋₁₀ heterocycloalkyl; the aryl, the heteroaryl and the heterocycloalkyl are optionally substituted by 0 to 4 substituents selected from F, CN, CF₃, hydroxy, C₁₋₆ alkyl, C₁₋₆ alkoxy or NR_{c}R_{d}; and the heteroaryl and the heterocycloalkyl each contain 1 to 4 heteroatoms selected from N, O or S.

R_{c} and R_{d} are independently selected from hydrogen, F, CN, hydroxy, C₁₋₆ alkyl, or C₁₋₆ alkoxy.

Z₁, Z₂, Z₃, Z₄, and Z₅ are each independently selected from N or —CR_{z}—.

R_{z} is selected from hydrogen, or C₁₋₆ alkyl.

R₁ and R₂ are independently selected from C₅₋₁₀ heteroaryl and C₆₋₁₀ aryl; the heterocycloalkyl and the aryl are each optionally substituted by 0 to 4 substituents selected from F, amino, aminoalkyl, aminocycloalkyl, aminoheterocyclyl, C₁₋₆ alkyl, C₁₋₆ alkoxy, C₅₋₁₀ heteroaryl, aryl, hydroxy, and CN; the heterocyclyl contains 1 to 3 heteroatoms selected from N, O or S. m is selected from 0.

Preferably, ring B is selected from or

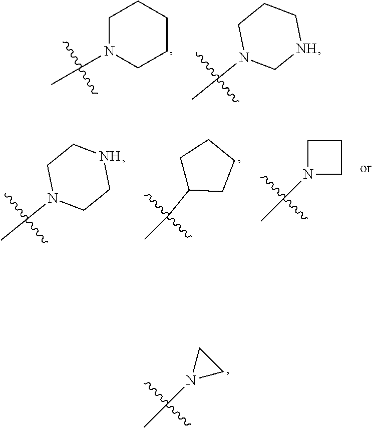

wherein the hydrogen of the group or

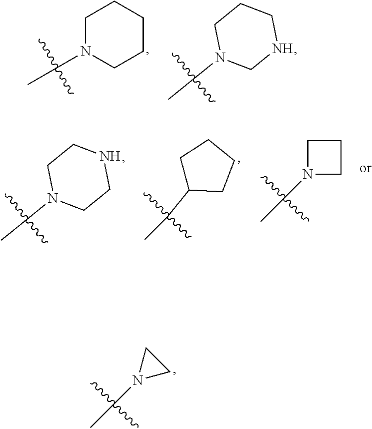

is optionally substituted by 0 to 4 substituents selected from NH₂, —NHCH₃ or methyl.
R_{z} is hydrogen;
R₁ is selected from benzene ring and thiazole, wherein the hydrogen of the benzene ring and the thiazole is further optionally substituted by 0 to 3 substituents selected from CN or F.
R₂ is selected from benzene ring and thiazole, wherein the hydrogen of the benzene ring and the thiazole is further optionally substituted by 0 to 3 substituents selected from methyl, methoxy or F.

The present disclosure provides a compound of formula (I-2),

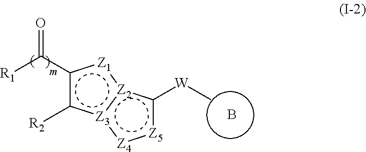

wherein the substituents are as defined above.

The present disclosure provides a compound of formula (I-2a),

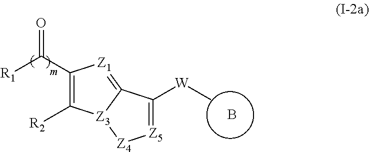

wherein, w is a bond.

In an aspect of the present disclosure, wherein Z₁ and Z₅ are each independently selected from N or ═CR_{z}—; Z₃ is N; and Z₄ is —NR₃—.

In an aspect of the present disclosure, wherein R_{z} is hydrogen.

In an aspect of the present disclosure, wherein ring A is

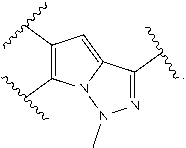 [SMILES: *c1cc2c(*)nn(C)n2c1*]

In an aspect of the present disclosure, wherein the compound is selected from the compound of formula (I-2b),

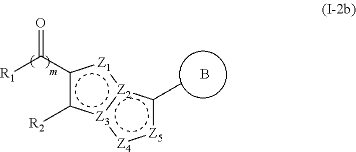

wherein the substituents are as defined above.

In an aspect of the present disclosure, wherein,
ring B is selected from C₆₋₁₀ aryl, C₃₋₁₀ heteroaryl, and C₃₋₁₀ heterocycloalkyl; the aryl, the heteroaryl and the heterocycloalkyl are optionally substituted by hydrogen, halogen, CN, hydroxy, C₁₋₆ alkyl, C₁₋₆ alkoxy or NR_{c}R_{d}; the heteroaryl and the heterocycloalkyl each contain 1 to 3 heteroatoms selected from N, O or S;
R_{c} and R_{d} are each independently selected from hydrogen, halogen, CN, hydroxy, C₁₋₆ alkyl, C₁₋₆ alkoxy, C₂₋₆ alkenyl or C₂₋₆ alkynyl;
Z₁, Z₂, Z₃, Z₄, and Z₅ are each independently selected from N, —NH— or —CR_{z}—;
R_{z} is selected from hydrogen or C₁₋₆ alkyl;
R₁ and R₂ are each independently selected from C₅₋₁₀ heteroaryl or C₆₋₁₀ aryl; wherein the C₅₋₁₀ heteroaryl or the aryl are each optionally substituted by 0 to 4 substituents selected from F, amino, aminoalkyl, aminocycloalkyl, aminoheterocyclyl, C₁₋₆ alkyl, C₁₋₆ alkoxy, C₅₋₁₀ heteroaryl, aryl, hydroxy or CN; and the heteroaryl contains 1 to 3 heteroatoms selected from N, O or S;
preferably, ring B is selected from

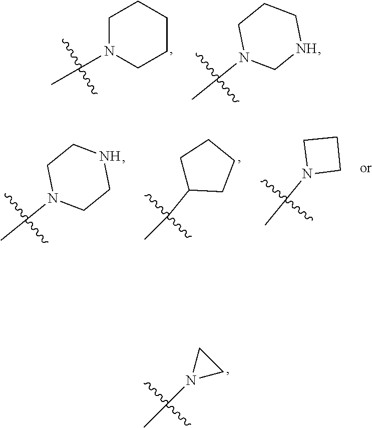

wherein the hydrogen of the group

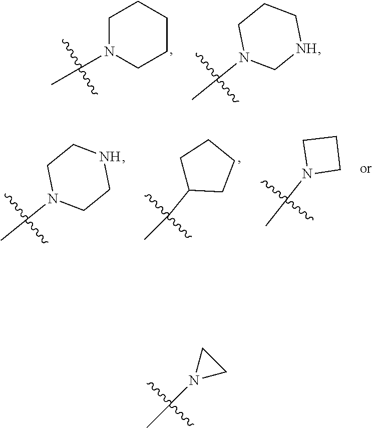

is optionally substituted by 0 to 2 substituents selected from —NH₂, —NHCH₃ or methyl;
R_{z} is selected from H or methyl;
R₁ is selected from benzene ring or thiazole, wherein the hydrogen of the benzene ring or thiazole is optionally substituted by 0 to 2 substituents selected from —CN or F;
R₂ is benzene ring, wherein the hydrogen of the benzene ring is optionally substituted by one or more substituents selected from methyl, methoxy or F.

The present disclosure provides a compound of formula (I-3),

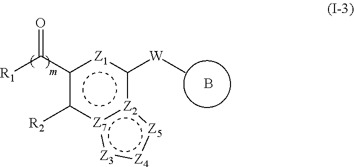

wherein the substituents are as defined above.

The present disclosure provides a compound of formula (I-3a),

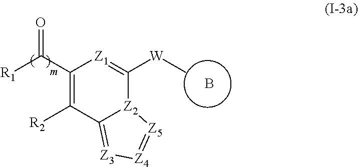

wherein Z₁, Z₂, Z₃, Z₄, and Z₅ are each independently selected from N or ═CR_{z}—; and W is a bond.

In an aspect of the present disclosure, wherein Z₂ is N; and Z₁, Z₃, Z₄, and Z₅ are each independently selected from N or ═CR_{z}—.

In an aspect of the present disclosure, wherein R_{z} is selected from hydrogen or C₁₋₆ alkyl.

In an aspect of the present disclosure wherein ring A is selected from

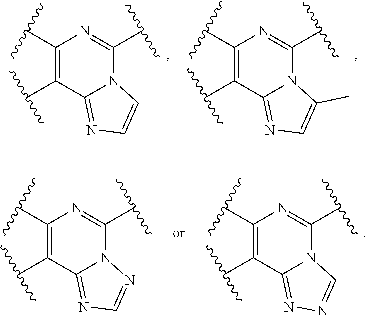 [SMILES: *c1nc(*)n2ccnc2c1* ; *c1nc(*)n2c(C)cnc2c1* ; *c1nc(*)n2ncnc2c1* ; *c1nc(*)n2cnnc2c1*]

The present disclosure provides a compound of formula (I-3b),

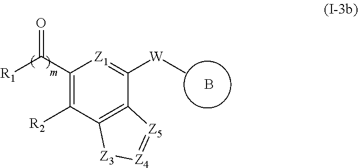

wherein Z₁, Z₄, and Z₅ are each independently selected from N or ═CR_{z}—; Z₃ is selected from O, S, —NR₃— or —CR_{y}R_{z}—; and W is a bond.

In an aspect of the present disclosure, wherein Z₁ is N; Z₃ is selected from O or —NR₃—; Z₄ and Z₅ are each independently selected from N or ═CR_{z}—; and W is a bond.

In an aspect of the present disclosure, wherein R_{z} is hydrogen.

In an aspect of the present disclosure, wherein ring A is selected from

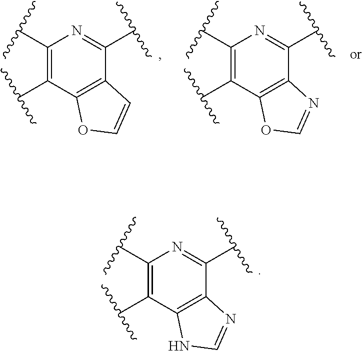 [SMILES: *c1nc(*)c2ccoc2c1* ; *c1nc(*)c2ncoc2c1* ; *c1nc(*)c2nc[nH]c2c1*]

The present disclosure provides a compound of formula (I-3c),

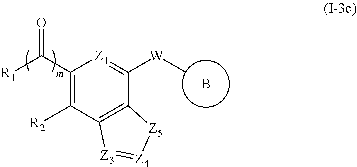

wherein Z₁, Z₃, and Z₄ are each independently selected from N or ═CR_{z}—; Z₅ is selected from O, S, —NR₃— or —CR_{y}R_{z}—; and W is a bond.

In an aspect of the present disclosure, wherein Z₁ is N; Z₃ and Z₄ are each independently selected from N or ═CR_{z}—; Z₅ is —NR₃—; and W is a bond.

In an aspect of the present disclosure, wherein R_{z} is hydrogen.

In an aspect of the present disclosure, wherein ring A is selected from

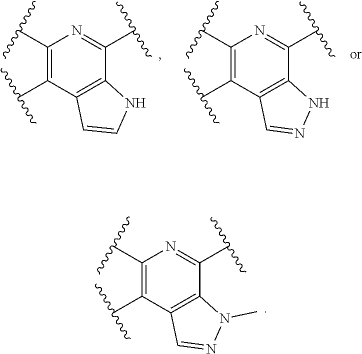 [SMILES: *c1nc(*)c2[nH]ccc2c1* ; *c1nc(*)c2[nH]ncc2c1* ; *c1nc(*)c2c(cnn2C)c1*]

The present disclosure provides a formula (I-3d),

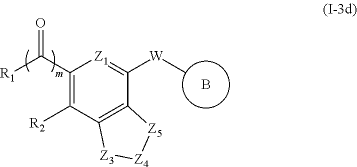

wherein Z₁ is selected from N or ═CR_{z}—; Z₃, Z₄, and Z₅ are each independently selected from O, S, —NR₃— or —CR_{y}R_{z}—; and W is a bond.

In an aspect of the present disclosure, wherein Z₁ is N; Z₃, Z₄, and Z₅ are each independently selected from O or —CR_{y}R_{z}—; and W is a bond.

In an aspect of the present disclosure, wherein R_{y} and R_{z} are both hydrogen.

In an aspect of the present disclosure, wherein ring A is

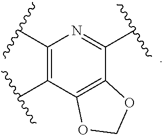 [SMILES: *c1nc(*)c2c(c1*)OCO2]

In an aspect of the present disclosure, wherein the compound is selected from the compound of formula (I-3e),

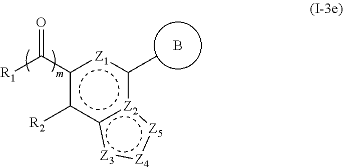

wherein the substituents are as defined above.

In an aspect of the present disclosure, wherein

ring B is selected from C₆₋₁₀ aryl, C₃₋₁₀ heteroaryl, and C₃₋₁₀ heterocycloalkyl; the aryl, the heteroaryl and the heterocycloalkyl are optionally substituted by 0 to 4 substituents selected from halogen, CN, hydroxy, C₁₋₆ alkyl, C₁₋₆ alkoxy or NR_{c}R_{d}; and the heteroaryl and the heterocycloalkyl each contains 1 to 3 heteroatoms selected from N, O or S.

R_{c} and R_{d} are each independently selected from hydrogen, halogen, CN, hydroxy, C₁₋₆ alkyl, C₁₋₆ alkoxy, C₂₋₆ alkenyl or C₂₋₆ alkynyl; and R_{c} and R_{d} are not simultaneously methyl.

Z₁, Z₂, Z₃, Z₄, and Z₅ are each independently selected from N or CR_{z}.

R_{z} is selected from hydrogen or C₁₋₆ alkyl.

R₁ and R₂ are each independently selected from C₅₋₁₀ heteroaryl or C₆₋₁₀ aryl; wherein the C₅₋₁₀ heteroaryl or the aryl are each optionally substituted by 0 to 4 substituents selected from F, amino, aminoalkyl, aminocycloalkyl, aminoheterocyclyl, C₁₋₆ alkyl, C₁₋₆ alkoxy, C₅₋₁₀ heteroaryl, aryl, hydroxy or CN; and the heteroaryl contains 1 to 3 heteroatoms selected from N, O or S.

m is 0.

preferably, ring B is selected from

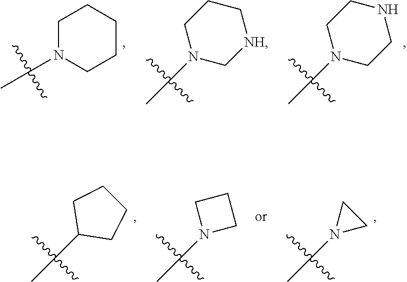

wherein the hydrogen of the group

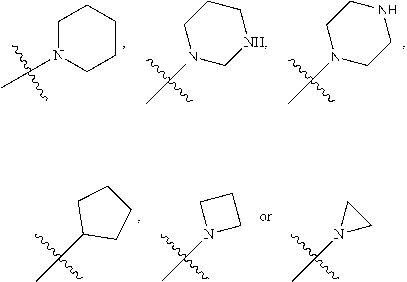

is optionally substituted by one or more substituents selected from NH₂, —NHCH₃ or methyl.

R_{z} is hydrogen.

R₁ is selected from benzene ring or thiazole, wherein the hydrogen of the benzene ring or the thiazole is further optionally substituted by 0 to 2 substituents selected from CN or F.

R₂ is selected from benzene ring, wherein the hydrogen of the benzene ring is optionally substituted by 0 to 2 substituents selected from methyl, methoxy or F.

In an aspect of the present disclosure, wherein the compound is selected from the compound of (I-4),

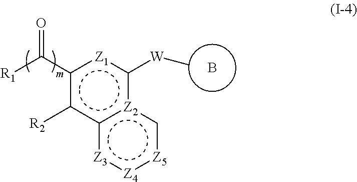

wherein the substituents are as defined above.

In an aspect of the present disclosure, wherein the compound is selected from the compound of formula (I-4a),

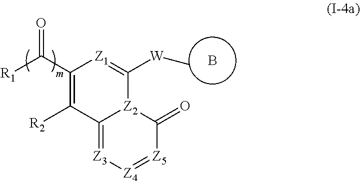

wherein, Z₁, Z₂, Z₃, Z₄, and Z₅ are each independently selected from N or ═CR_{z}—; and W is a bond.

In an aspect of the present disclosure, wherein R_{z} is hydrogen.

In an aspect of the present disclosure, wherein ring A is

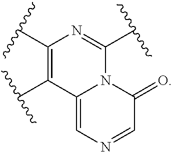 [SMILES: *c1nc(*)n2c(=O)cncc2c1*]

In an aspect of the present disclosure, wherein the compound is selected from the compound of formula (I-4b),

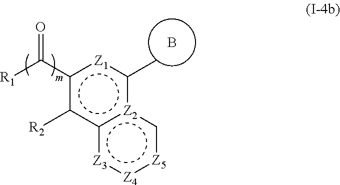

wherein the substituents are as defined above.

In an aspect of the present disclosure, wherein
ring B is selected from C₆₋₁₀ aryl, C₃₋₁₀ heteroaryl or C₃₋₁₀ heterocycloalkyl; the aryl, the heteroaryl or the heterocycloalkyl are optionally substituted by hydrogen, halogen, CN, hydroxy, C₁₋₆ alkyl, C₁₋₆ alkoxy or —NR_{c}R_{d}; and the heteroaryl and the heterocycloalkyl each contain 1 to 3 heteroatoms selected from N, O or S.
R_{c} and R_{d} are each independently selected from hydrogen, halogen, CN, hydroxy, C₁₋₆ alkyl, C₁₋₆ alkoxy, C₂₋₆ alkenyl or C₂₋₆ alkynyl;
Z₁, Z₂, Z₃, Z₄, and Z₅ are each independently selected from N, —NH— or —CR_{z}—;
R_{z} is selected from hydrogen or C₁₋₆ alkyl;
R₁ and R₂ are each independently selected from C₅₋₁₀ heteroaryl or C₆₋₁₀ aryl; wherein the C₅₋₁₀ heteroaryl and aryl are each optionally substituted by 0 to 4 substituents selected from F, amino, aminoalkyl, aminocycloalkyl, aminoheterocyclyl, C₁₋₆ alkyl, C₁₋₆ alkoxy, C₅₋₁₀ heteroaryl, aryl, hydroxy or CN; the heteroaryl contains 1 to 3 heteroatoms selected from N, O or S;
m is 0;
preferably, ring B is selected from

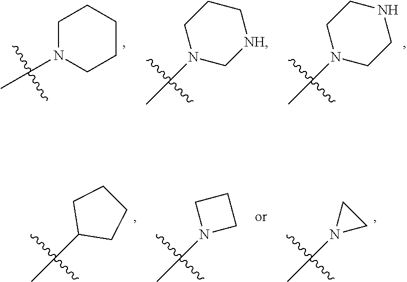

wherein the hydrogen of the group

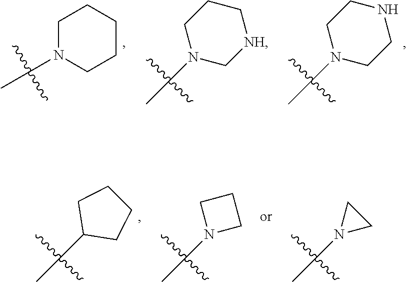

is optionally substituted by 0 to 2 substituents selected from —NH₂, —NHCH₃ or methyl.
R_{z} is hydrogen;
R₁ is selected from benzene ring or thiazole, wherein the hydrogen of the benzene ring or thiazole is optionally substituted by 0 to 2 substituents selected from —CN or F;
R₂ is benzene ring, wherein the hydrogen of the benzene ring is optionally substituted by 0 to 2 substituents selected from methyl, methoxy or F.

In an aspect of the present disclosure, wherein the compound is selected from the compound of formula (I-5),

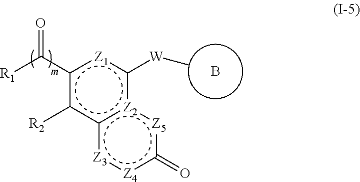

wherein the substituents are as defined above.

In an aspect of the present disclosure, wherein the compound is selected from the compound of formula (I-5a),

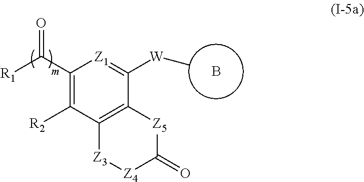

wherein Z₁ is selected from N or ═CR_{z}—; Z₃, Z₄, and Z₅ are each independently selected from O, S, —NR₃— or —CR_{y}R_{z}—; and W is a bond.

In an aspect of the present disclosure, wherein Z₁ is N; Z₃, Z₄, and Z₅ are each independently selected from O, —NR₃— or —CR_{y}R_{z}—; and W is a bond.

In an aspect of the present disclosure, wherein R_{y} and R_{z} are both hydrogen.

In an aspect of the present disclosure, wherein ring A is

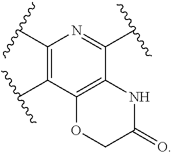 [SMILES: *c1nc(*)c2c(c1*)OCC(=O)N2]

In an aspect of the present disclosure, wherein the compound is selected from the compound of formula (I-6),

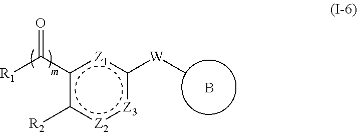

wherein the substituents are as defined above.

In an aspect of the present disclosure, wherein the compound is selected from the compound of formula (I-6a),

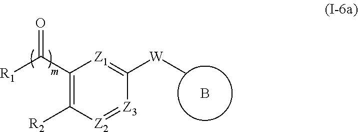

wherein Z₁, Z₂, and Z₃ are each independently selected from N or ═CR_{z}—.

In an aspect of the present disclosure, wherein W is selected from a bond,

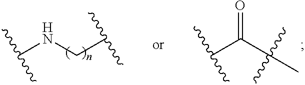

and n is selected from 0 or 1.

In an aspect of the present disclosure, wherein ring A is selected from:

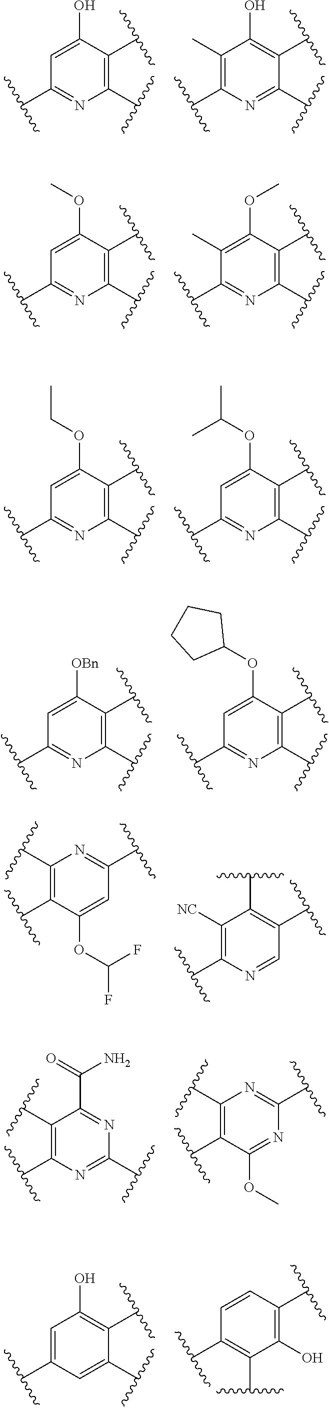

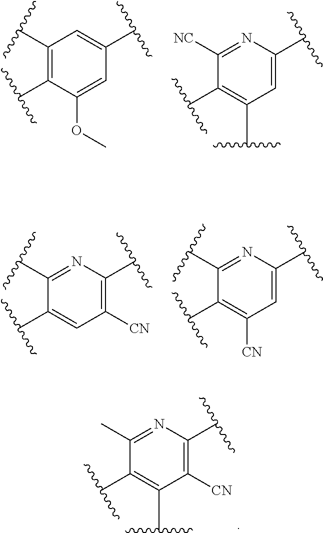 [SMILES: *c1cc(*)c(*)c(OC)c1 ; *c1cc(*)c(*)c(C#N)n1 ; *c1cc(C#N)c(*)nc1* ; *c1cc(C#N)c(*)c(*)n1 ; *c1nc(C)c(*)c(*)c1C#N]

In an aspect of the present disclosure, wherein the compound is selected from the compound of formula (I-6b),

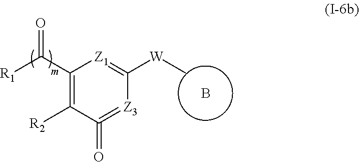

wherein Z₁ is selected from N or ═CR_{z}—; and Z₃ is selected from —NR₃— or —CR_{y}R_{z}—.

In an aspect of the present disclosure, wherein Z₁ is N, and Z₃ is —NR₃—.

In an aspect of the present disclosure, wherein W is —NH—.

In an aspect of the present disclosure, wherein ring A is

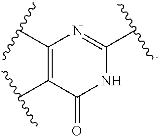 [SMILES: *c1nc(*)c(*)c(=O)[nH]1]

In an aspect of the present disclosure, wherein the compound is selected from the compound of formula (I-6c),

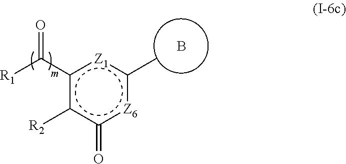

wherein the substituents are as defined above.

In an aspect of the present disclosure, wherein
ring B is selected from C₆₋₁₀ aryl, C₃₋₁₀ heteroaryl or C₃₋₁₀ heterocycloalkyl; the aryl, the heteroaryl, and the heterocycloalkyl are each optionally substituted by 0 to 4 substituents selected from F, CN, hydroxy, C₁₋₆ alkyl, C₁₋₆ alkoxy or NR_{c}R_{d}; and the heteroaryl and the heterocycloalkyl each contain 1 to 3 heteroatoms selected from N, O or S;
R_{c} and R_{d} are each independently selected from hydrogen, halogen, CN, hydroxy, C₁₋₆ alkyl, C₁₋₆ alkoxy, C₂₋₆ alkenyl or C₂₋₆ alkynyl;
Z₁, Z₂, Z₃, Z₄, and Z₅ are each independently selected from N, —NH— or —CR_{z}—; R_{z} is hydrogen;
R₁ and R₂ are each independently selected from C₅₋₁₀ heteroaryl or C₆₋₁₀ aryl; wherein the C₅₋₁₀ heteroaryl or the aryl are each optionally substituted by 0 to 2 substituents selected from F, amino, aminoalkyl, aminocycloalkyl, aminoheterocyclyl, C₁₋₆ alkyl, C₁₋₆ alkoxy, C₅₋₁₀ heteroaryl, aryl, hydroxy, and CN; and the heteroaryl contains 1 to 3 heteroatoms selected from N, O or S;
m is 0;
preferably, ring B is selected from

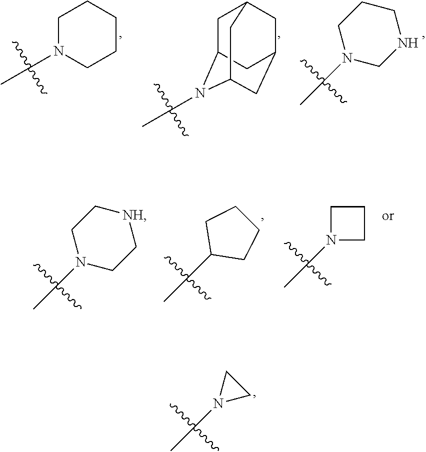

wherein the hydrogen of the group

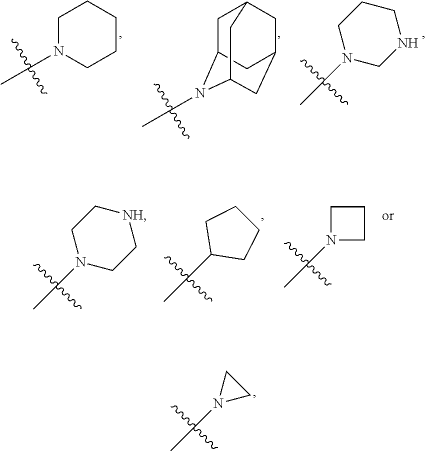

is optionally substituted by one or more substituents selected from —NH₂, hydroxy, —NHCH₃ or methyl;
R₁ is selected from benzene ring or thiazole, wherein the hydrogen of the benzene ring or the thiazole is optionally substituted by 0 to 2 substituents selected from CN or F; and
R₂ is selected from benzene ring, wherein the hydrogen of the benzene ring is optionally substituted by 0 to 2 substituents selected from methyl, methoxy or F.

In an aspect of the present disclosure, wherein the compound is selected from the compound of formula (I-6d),

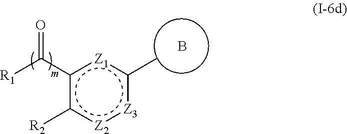

wherein the substituents are as defined above.

In an aspect of the present disclosure, wherein
ring B is selected from C₆₋₁₀ aryl, C₃₋₁₀ heteroaryl or C₃₋₁₀ heterocycloalkyl; the aryl, the heteroaryl, and the heterocycloalkyl are each optionally substituted by 0 to 4 substituents selected from F, CN, hydroxy, C₁₋₆ alkyl, C₁₋₆ alkoxy or —NR_{c}R_{d}; and the heteroaryl and the heterocycloalkyl each contain 1 to 3 heteroatoms selected from N, O or S;
R_{c} and R_{d} are each independently selected from hydrogen, halogen, CN, hydroxy, C₁₋₆ alkyl, C₁₋₆ alkoxy, C₂₋₆ alkenyl or C₂₋₆ alkynyl;
Z₁, Z₂, Z₃, Z₄, and Z₅ are each independently selected from N or CR_{z};
R_{z} is selected from hydrogen, hydroxy, C₁₋₆ alkoxy or

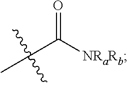

Rₐ and R_{b} are each selected from H;
R₁ and R₂ are each independently selected from C₅₋₁₀ heteroaryl or C₆₋₁₀ aryl; wherein the C₅₋₁₀ heteroaryl and the aryl are each optionally substituted by 0 to 4 substituents selected from F, amino, aminoalkyl, aminocycloalkyl, aminoheterocyclyl, C₁₋₆ alkyl, C₁₋₆ alkoxy, C₅₋₁₀ heteroaryl, aryl, hydroxy or —CN; and the heterocyclyl contains 1 to 3 heteroatoms selected from N, O or S;
m is 0;
preferably, ring B is selected from

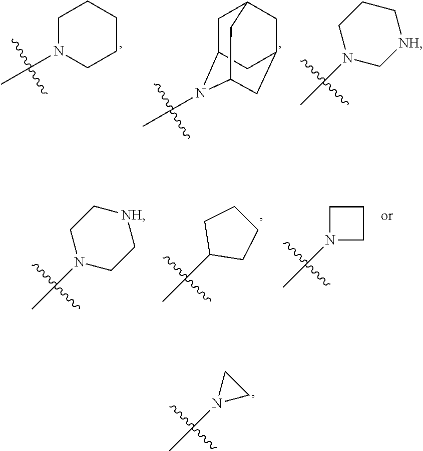

wherein the hydrogen of the group

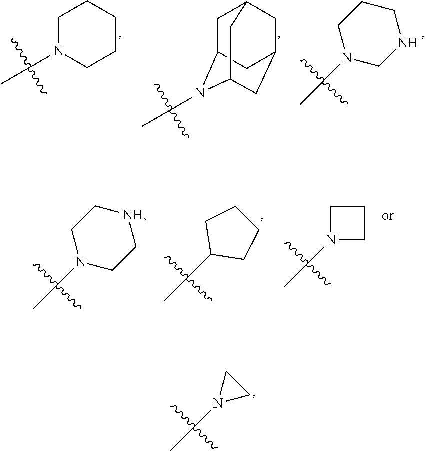

is optionally substituted by one or more substituents selected from —NH₂, hydroxy, —NHCH₃ or methyl;
R_{z} is selected from hydrogen, hydroxy, methoxy or

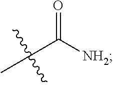 [SMILES: *C(N)=O]

R₁ is selected from benzene ring or thiazole, wherein the hydrogen of the benzene ring and the thiazole is optionally substituted by 0 to 2 substituents selected from —CN or F; and
R₂ is selected from benzene ring, wherein the hydrogen of the benzene ring is optionally substituted by 0 to 2 substituents selected from methyl, methoxy or F.

In an aspect of the present disclosure, wherein the compound is selected from the compound of formula (I-7), wherein

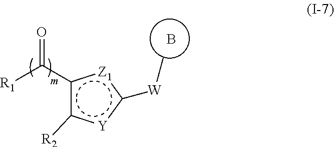

wherein the substituents are as defined above.

In an aspect of the present disclosure, wherein the compound is selected from the compound of (I-7a),

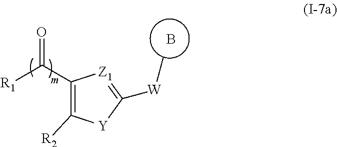

wherein Z₁ is selected N or ═CR_{z}—.

In an aspect of the present disclosure, wherein R_{z} is hydrogen.

In an aspect of the present disclosure, wherein W is selected from a bond,

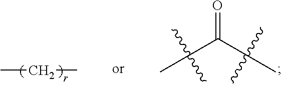

and r is 1.

In an aspect of the present disclosure, wherein ring A is selected from

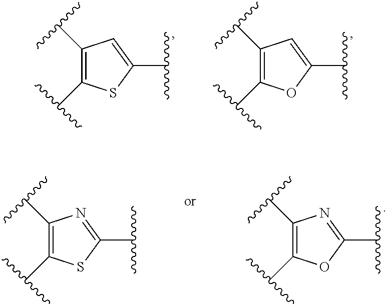 [SMILES: *c1cc(*)c(*)s1 ; *c1cc(*)c(*)o1 ; *c1nc(*)c(*)s1 ; *c1nc(*)c(*)o1]

In an aspect of the present disclosure, wherein the compound is selected from the compound of formula (I-7b), wherein

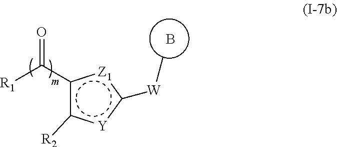

wherein the substituents are as defined above.

In an aspect of the present disclosure, wherein ring B is selected from C₆₋₁₀ aryl, C₃₋₁₀ heteroaryl or C₃₋₁₀ heterocycloalkyl; the aryl, the heteroaryl, and the heterocycloalkyl are each optionally substituted by 0 to 4 substituents selected from F, CN, hydroxy, C₁₋₆ alkyl, C₁₋₆ alkoxy or —NR_{c}R_{d}; and the heteroaryl and the heterocycloalkyl each contain 1 to 3 heteroatoms selected from N, O or S.
R_{c} and R_{d} are each independently selected from hydrogen, halogen, —CN, hydroxy, C₁₋₆ alkyl, C₁₋₆ alkoxy, C₂₋₆ alkenyl or C₂₋₆ alkynyl;
W is selected from —CH₂— or

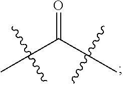 [SMILES: *C(*)=O]

Z₁ is selected from N or CR_{z};
R_{z} is selected from hydrogen or C₁₋₆ alkyl;
Y is selected from O or S;
R₁ and R₂ are each independently selected from C₁₋₆ alkyl, C₅₋₁₀ heteroaryl or C₆₋₁₀ aryl; the C₅₋₁₀ heteroaryl and the aryl are each optionally substituted by 0 to 4 substituents selected from F, amino, aminoalkyl, aminocycloalkyl, aminoheterocyclyl, C₁₋₆ alkyl, C₁₋₆ alkoxy, C₅₋₁₀ heteroaryl, aryl, hydroxy or CN; and the heteroaryl contains 1 to 3 heteroatoms selected from N, O or S;
m is 0;
preferably, ring B is selected from

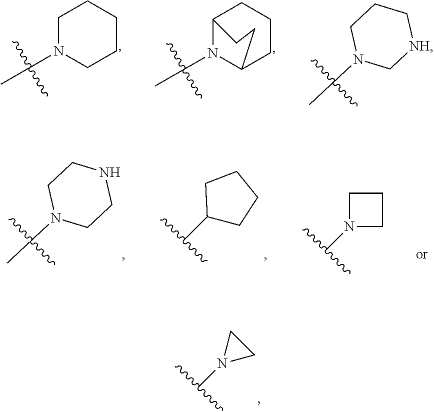

wherein the hydrogen of the group

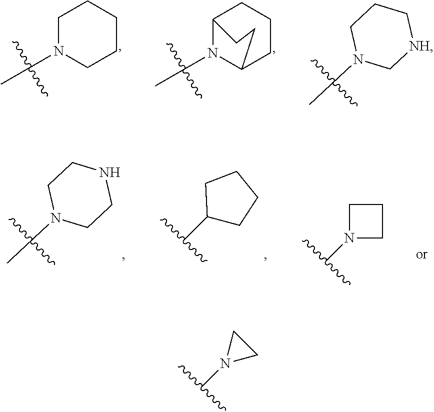

is optionally substituted by one or more substituents selected from —NH₂, hydroxy, —NHCH₃ or methyl;
Z₁ is selected from CR_{z};
R_{z} is hydrogen;
R₁ is selected from methyl, benzene ring, pyrimidine, pyridine or thiazole, wherein the hydrogen of the methyl, the benzene ring or the thiazole is optionally substituted by 0 to 2 substituents selected from CN or F; and
R₂ is selected from methyl, benzene ring, pyridine, pyrimidine,

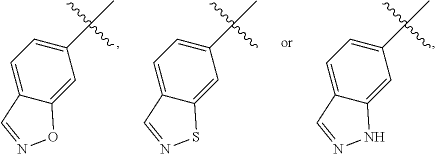 [SMILES: *c1ccc2cnoc2c1 ; *c1ccc2cnsc2c1 ; *c1ccc2cn[nH]c2c1]

wherein the hydrogen of the methyl, the benzene ring, the pyridine, the pyrimidine,

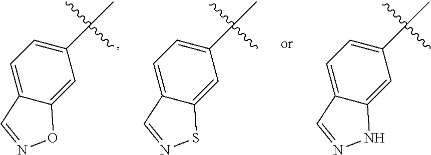 [SMILES: *c1ccc2cnoc2c1 ; *c1ccc2cnsc2c1 ; *c1ccc2cn[nH]c2c1]

is optionally substituted by 0 to 2 substituents selected from methyl, methoxy or F.

In an aspect of the present disclosure, wherein the compound is selected from the compound or formula (I-8),

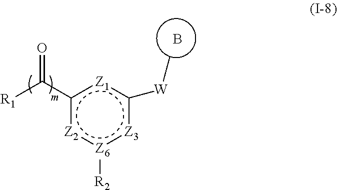

wherein the substituents are as defined above.

In an aspect of the present disclosure, wherein the compound is selected from the compound of formula (I-8a),

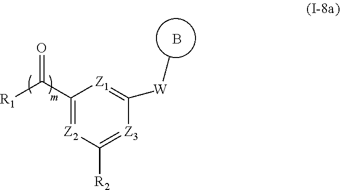

wherein Z₁, Z₂, and Z₃ are each independently selected from N or ═CR_{z}—.

In an aspect of the present disclosure, wherein R_{z} is selected from hydrogen or C₁₋₆ alkoxy.

In an aspect of the present disclosure, wherein W is selected from a bond,

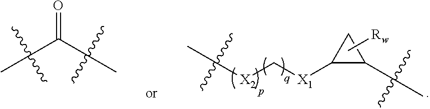

In an aspect of the present disclosure, wherein X₂ is —O—; p is selected from 0 or 1; q is selected from 1, 2, 3, or 4; X₁ is —NH—; and R_{W} is H.

In an aspect of the present disclosure, wherein ring A is selected from

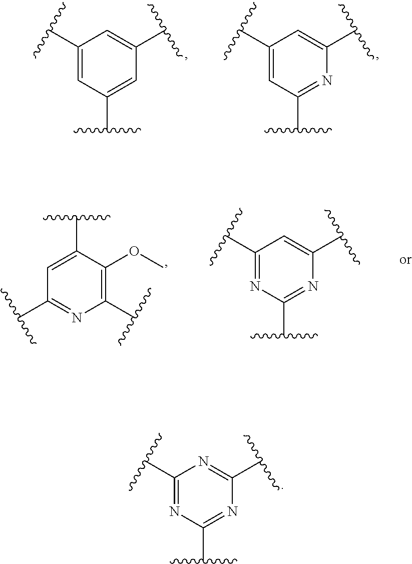 [SMILES: *c1cc(*)cc(*)c1 ; *c1cc(*)nc(*)c1 ; *c1cc(*)c(OC)c(*)n1 ; *c1cc(*)nc(*)n1 ; *c1nc(*)nc(*)n1]

In an aspect of the present disclosure, wherein the compound is selected from the compound of formula (I-8b),

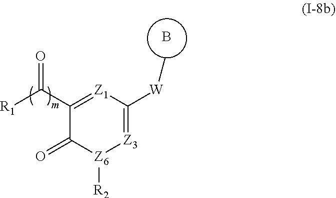

wherein Z₁ and Z₃ are each independently selected from N or ═CR_{z}—; and Z₆ is selected from N or

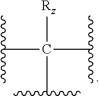

In an aspect of the present disclosure, wherein R_{z} is selected from hydrogen or C₁₋₆ alkyl.

In an aspect of the present disclosure, wherein W is

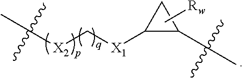

In an aspect of the present disclosure, wherein p is 0; q is 3; X₁ is —NH—; and R_{W}, is H.

In an aspect of the present disclosure, wherein ring A is

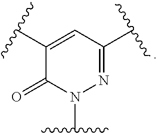 [SMILES: *c1cc(*)c(=O)n(*)n1]

In an aspect of the present disclosure, wherein ring B is selected from C₆₋₁₀ aryl, C₃₋₁₀ cycloalkyl or C₂₋₁₀ heterocycloalkyl; the heterocycloalkyl contains 1 to 2 N heteroatoms; the cycloalkyl and the heterocycloalkyl are monocyclic, bicyclic or polycyclic group; the bicyclic and polycyclic group are bridge ring or fused ring; and
the hydrogen of the aryl, cycloalkyl and heterocycloalkyl ring is optionally substituted by 0 to 2 substituents selected from halogen, —CN, hydroxy, C₁₋₆ alkyl, C₁₋₆ alkoxy, C₂₋₁₀ heterocycloalkyl or —NR_{c}R_{d}, wherein the C₂₋₁₀ heterocycloalkyl as a substituent contains 1 to 2 heteroatoms selected from N or O; and R_{c} and R_{d} are each independently selected from H, C₁₋₆ alkyl, hydroxy substituted C₁₋₆ alkyl, and C₁₋₆ alkoxy substituted C₁₋₆ alkyl.

In an aspect of the present disclosure, wherein ring B is selected from

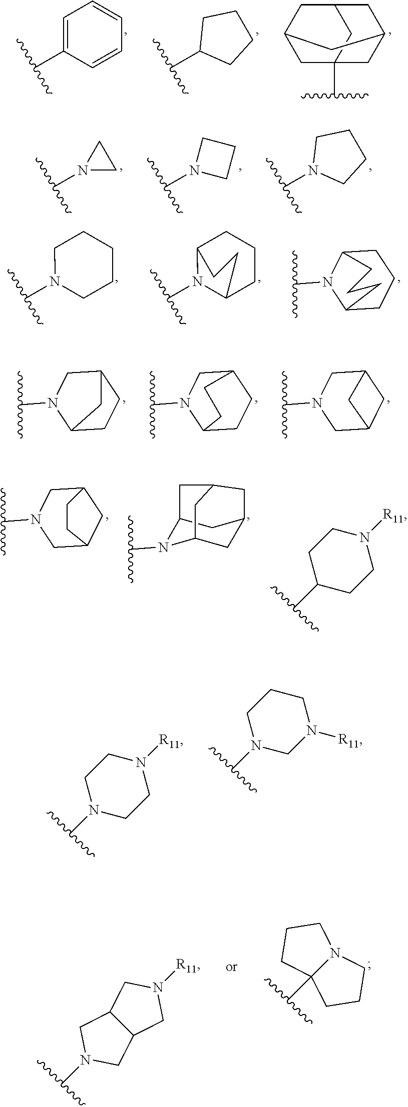

R₁₁ is selected from H, or C₁₋₆ alkyl, and the hydrogen of the ring is optionally substituted by 0 to 2 substituents selected from halogen, —CN, hydroxy, C₁₋₆ alkyl, C₁₋₆ alkoxy, 6-membered heterocycloalkyl or —NRₑR_{d}, wherein the 6-membered heterocycloalkyl as a substituent contains 1 to 2 heteroatoms selected from N and 0; and R_{c} and R_{d} are each independently selected from H, C₁₋₆ alkyl, hydroxy substituted C₁₋₆ alkyl, and C₁₋₃ alkoxy substituted C₁₋₃ alkyl.

In an aspect of the present disclosure, wherein R_{c} and R_{d} are each independently selected from H, methyl,

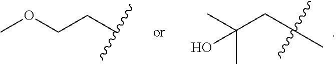

In an aspect of the present disclosure, wherein the hydrogen of the ring is optionally substituted by 0 to 2 substituents selected from —F, —CN, hydroxy, methyl, methoxy, —NH₂, —NHCH₃, —N(CH₃)₂,

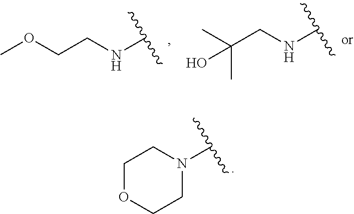

In an aspect of the present disclosure, wherein the compound is selected from the compound of formula (I-8c),

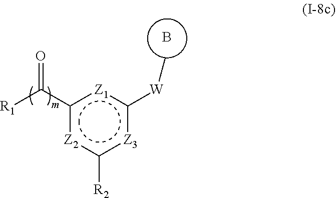

wherein the substituents are as defined above.

In an aspect of the present disclosure, wherein
ring B is selected from C₆₋₁₀ aryl, C₃₋₁₀ heteroaryl or C₃₋₁₀ heterocycloalkyl; the aryl, the heteroaryl and the heterocycloalkyl are optionally substituted by hydrogen, halogen, CN, hydroxy, C₁₋₆ alkyl, C₁₋₆ alkoxy or NR_{c}R_{d}; the heteroaryl and the heterocycloalkyl each contain 1 to 3 heteroatoms selected from N, O or S;
R_{c} and R_{d} are each independently selected from hydrogen, halogen, —CN, hydroxy, C₁₋₆ alkyl, C₁₋₆ alkoxy, C₂₋₆ alkenyl or C₂₋₆ alkynyl;
W is selected from

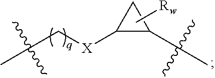

X is selected from —NRₓ—;
R_{W} and Rₓ are each independently selected from H, C₁₋₆ alkyl, C₁₋₆ alkoxy, halogen, C₂₋₆ alkenyl or C₂₋₆ alkynyl;
Z₁, Z₂, and Z₃ are each independently selected from N or CR_{z};
R_{z} is selected from hydrogen or C₁₋₆ alkyl;
R₁ and R₂ are each independently selected from C₅₋₁₀ heteroaryl or C₆₋₁₀ aryl; the C₅₋₁₀ heteroaryl and aryl are each optionally substituted by 0 to 4 substituents selected from F, amino, aminoalkyl, aminocycloalkyl, aminoheterocyclyl, C₁₋₆ alkyl, C₁₋₆ alkoxy, C₅₋₁₀ heteroaryl, aryl, hydroxy, CN or S(O)₂Rₐ; and the heteroaryl contains 1 to 3 heteroatoms selected from N, O or S;
Rₐ is selected from C₁₋₆ alkyl;
m is 1;
q is selected from 1, 2, 3 or 4;
preferably, ring B is selected from benzene ring,

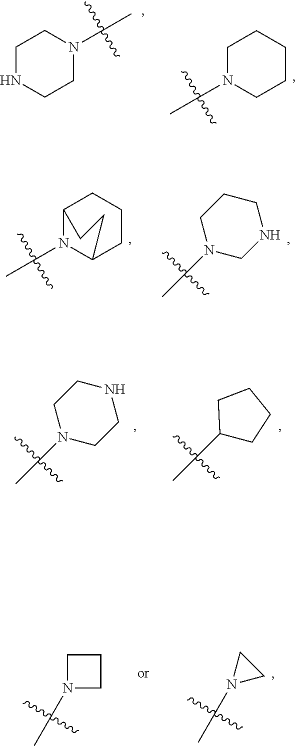

wherein the hydrogen of the benzene ring,

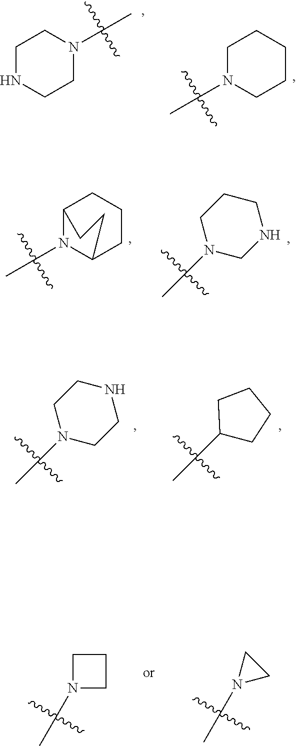

is optionally substituted by 0 to 4 substituents selected from —NH₂, CN, F or methyl;
X is —NH—;
R_{W} is selected from H, C₁₋₆ alkyl, C₁₋₆ alkoxy or halogen;
R_{z} is hydrogen;
R₁ is selected from

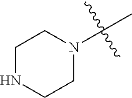 [SMILES: *N1CCNCC1]

or benzene ring, wherein the hydrogen of

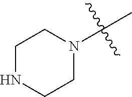 [SMILES: *N1CCNCC1]

and the benzene ring is optionally substituted by 0 to 2 substituents selected from methyl,

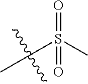 [SMILES: *S(C)(=O)=O]

or methoxy; and
R₂ is selected from benzene ring or

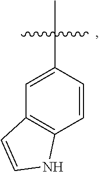 [SMILES: *c1ccc2[nH]ccc2c1]

wherein the hydrogen of the benzene ring and

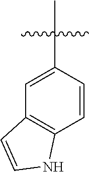 [SMILES: *c1ccc2[nH]ccc2c1]

is optionally substituted by 0 to 2 substituents selected from methyl, methoxy, F or

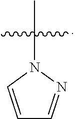 [SMILES: *n1cccn1]

In an aspect of the present disclosure, wherein ring B is selected from

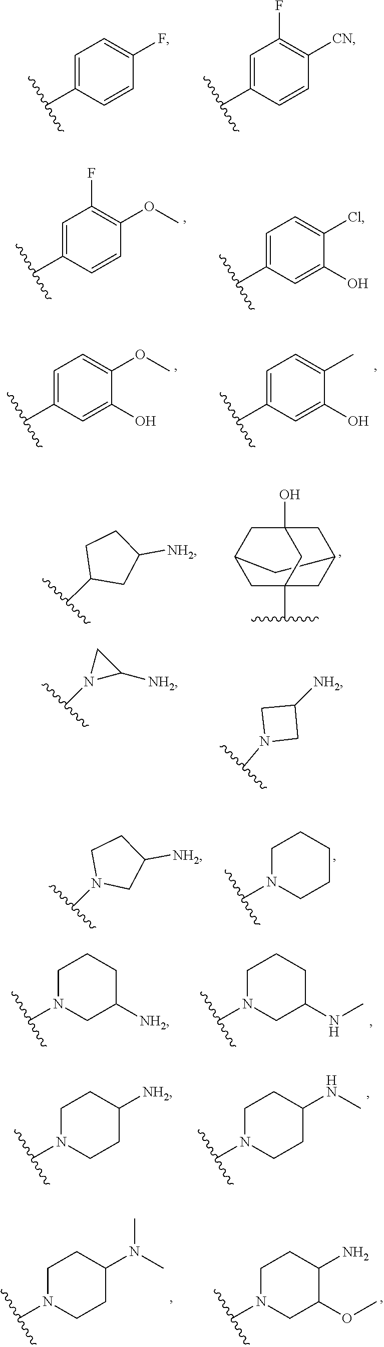

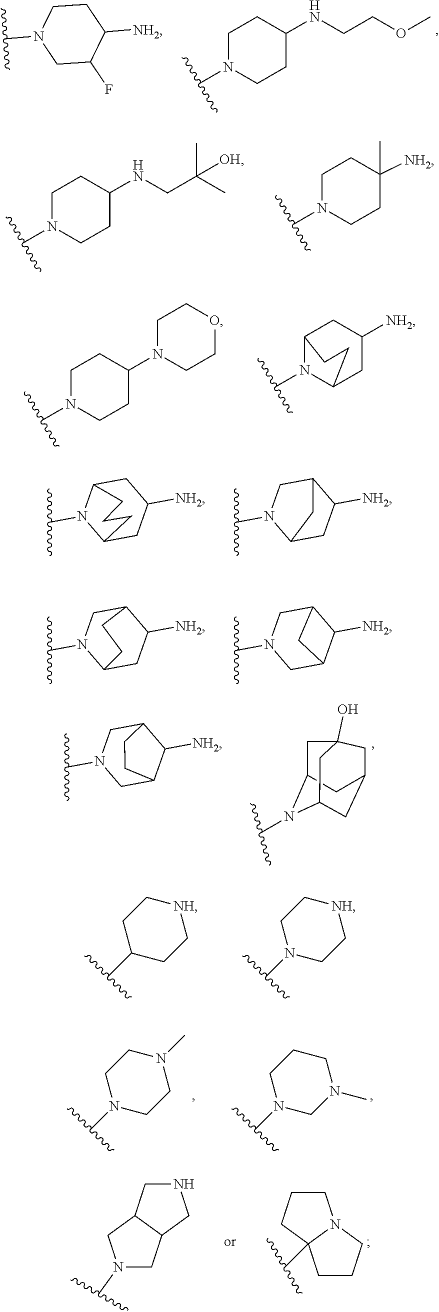

and
preferably, ring B is selected from:

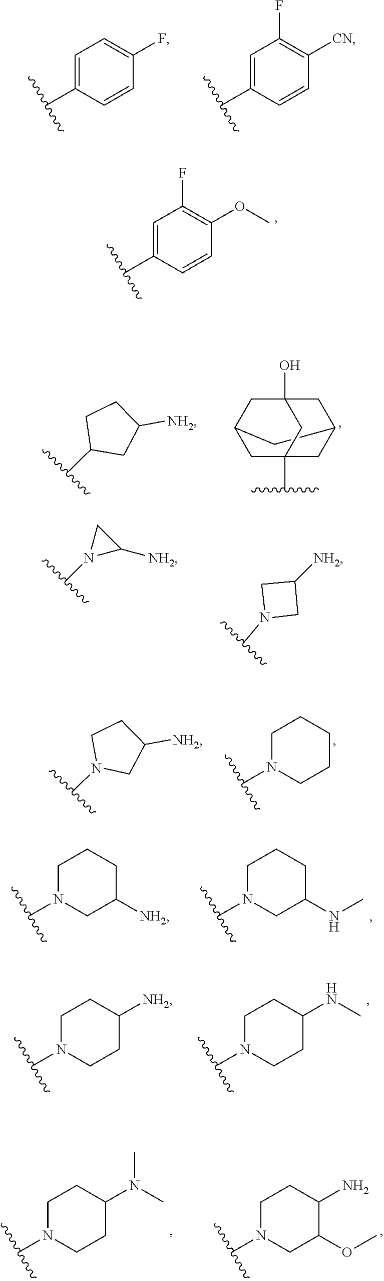

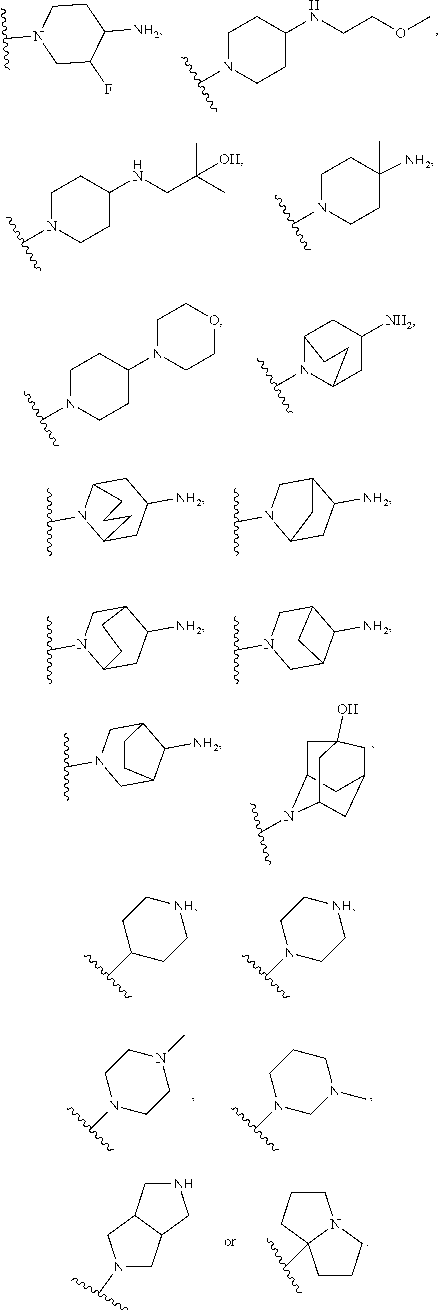

In an aspect of the present disclosure, wherein ring B is selected from

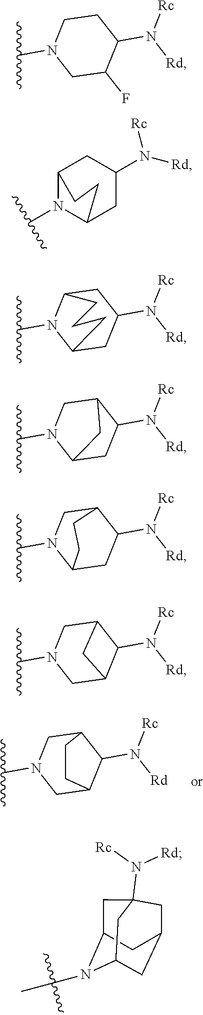

preferably, Rₑ and R_{d} are each independently selected from H, C₁₋₆ alkyl, hydroxy substituted C₁₋₆ alkyl, or C₁₋₆ alkoxy substituted C₁₋₆ alkyl; and further preferably, R_{c} and R_{d} are both hydrogen.

In an aspect of the present disclosure, wherein one of R₁ and R₂ is hydrogen.

In an aspect of the present disclosure, wherein one of R₁ and R₂ is C₁₋₆ alkyl optionally substituted by 0 to 1 substituent selected from C₆₋₁₀ aryl, 5-10-membered heteroaryl, C₃₋₁₀ cycloalkyl or C₂₋₁₀ heterocycloalkyl; the C₆₋₁₀ aryl, the 5-10-membered heteroaryl, the C₃₋₁₀ cycloalkyl, and the C₂₋₁₀ heterocycloalkyl are further optionally substituted by 0 to 2 substituents selected from halogen or C₁₋₆ alkoxy; the 5-10-membered heteroaryl and the C₂₋₁₀ heterocycloalkyl each contain 1 to 3 heteroatoms selected from N, O or S;
preferably, one of R₁ and R₂ is methyl optionally substituted by 0 to 1 substituent selected from phenyl and 6-membered heterocycloalkyl; the phenyl and the 6-membered heterocycloalkyl are further optionally substituted by 0 to 2 substituents selected from halogen and C₁₋₆ alkoxy; the 6-membered heterocycloalkyl contains 1 to 2 heteroatoms selected from N or O; and
further preferably, one of R₁ and R₂ is selected from methyl,

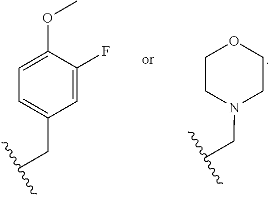 [SMILES: *Cc1ccc(OC)c(F)c1 ; *CN1CCOCC1]

In an aspect of the present disclosure, wherein one of R₁ and R₂ is C₂₋₆ alkynyl optionally substituted by 0 to 1 substituent selected from C₆₋₁₀ aryl or 5-10-membered heteroaryl; the C₆₋₁₀ aryl, and the 5-10-membered heteroaryl are further optionally substituted by 0 to 2 substituents selected from halogen and C₁₋₆ alkoxy; the 5-10-membered heteroaryl contains 1 to 3 heteroatoms selected from N, O or S;
preferably, one of R₁ and R₂ is acetenyl optionally substituted by 0 to 1 phenyl; the phenylis further optionally substituted by 0 to 2 substituents selected from halogen or C₁₋₆ alkoxy; and
further preferably, one of R₁ and R₂ is

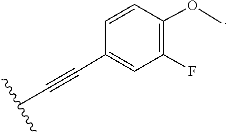 [SMILES: *C#Cc1ccc(OC)c(F)c1]

In an aspect of the present disclosure, wherein R₁ and/or R₂ is C₂₋₁₀ heterocycloalkyl; the heterocycloalkyl is optionally substituted by 0 to 2 substituents selected from C₁₋₆ alkyl, —S(O)₂R₆, halogen, —COOH or —C(═O)OC₁₋₆ alkyl; R₆ is selected from C₁₋₆ alkyl or —NH₂; the heterocycloalkyl contains 1 to 2 heteroatoms selected from N or O; preferably, the heterocycloalkyl is a 6-membered heterocycloalkyl; and further preferably, R₁ and/or R₂ is selected from

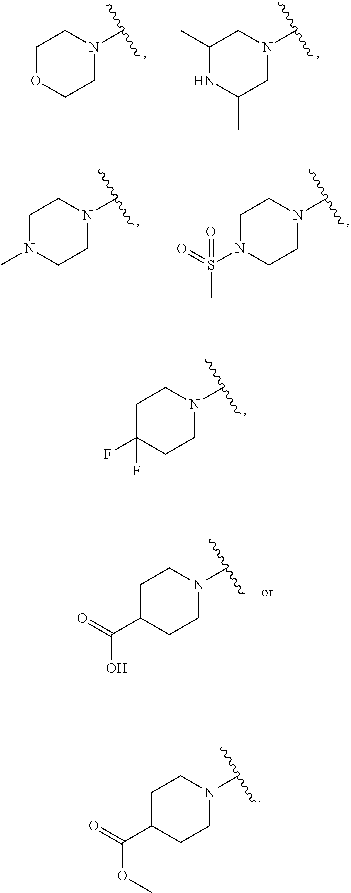

In an aspect of the present disclosure, wherein R₁ and/or R₂ is selected from C₆₋₁₀ aryl, 5-10-membered heteroaryl, —OC₆₋₁₀ aryl, —O(5-10-membered heteroaryl), and C₆₋₁₀ aryl fused C₂₋₁₀ heterocycloalkyl; the C₆₋₁₀ aryl, the 5-10-membered heteroaryl, the —OC₆₋₁₀ aryl, the —O(5-10-membered heteroaryl), and the C₆₋₁₀ aryl fused C₂₋₁₀ heterocycloalkyl are optionally substituted by 0 to 4 substituents selected from —CN, —CH₂CN, halogen, —S(O)₂R₆, C₁₋₆ alkyl, halogen substituted C₁₋₆ alkyl, hydroxy, C₁₋₆ alkoxy, halogen substituted C₁₋₆ alkoxy, —NH₂, hydroxy substituted C₁₋₆ alkyl, hydroxy substituted C₂₋₆ alkynyl, hydroxy substituted C₁₋₆ alkoxy, 5-10-membered heteroaryl or C₂₋₁₀ heterocycloalkyl; the C₆₋₁₀ aryl fused C₂₋₁₀ heterocycloalkyl can be substituted by ═O; the heteroaryl and the heterocycloalkyl each contain 1 to 3 heteroatoms selected from N, O or S; and R₆ is selected from C₁₋₆ alkyl or —NH₂;
preferably, R₁ and/or R₂ is selected from phenyl, 5-10-membered heteroaryl, phenoxy, and phenyl fused 5-membered heterocycloalkyl; the phenyl, the 5-10-membered heteroaryl, the phenoxy or the phenyl fused 5-membered heterocycloalkyl are optionally substituted by 0 to 4 substituents selected from —CN, —CH₂CN, halogen, —S(O)₂R₆, C₁₋₆ alkyl, halogen substituted C₁₋₆ alkyl, hydroxy, C₁₋₆ alkoxy, halogen substituted C₁₋₆ alkoxy, —NH₂, hydroxy substituted C₁₋₆ alkyl, hydroxy substituted C₂₋₆ alkynyl, hydroxy substituted C₁₋₆ alkoxy, 5-6-membered heteroaryl or 5-6-membered heterocycloalkyl; the phenyl fused 5-membered heterocycloalkyl can be substituted by ═O; the heteroaryl and the heterocycloalkyl each contain 1 to 3 heteroatoms selected from N, O or S; and R₆ is selected from C₁₋₆ alkyl or —NH₂;
preferably, R₁ and/or R₂ is selected from phenyl, 5-10-membered heteroaryl, phenoxy and phenyl fused 5-membered heterocycloalkyl; the phenyl, the 5-10-membered heteroaryl, the phenoxy or the phenyl fused 5-membered heterocycloalkyl are optionally substituted by 0 to 4 substituents selected from —CN, —CH₂CN, F, —C₁, —S(O)₂R₆, methyl, pentyl, trifluoromethyl, hydroxy, methoxy, trifluoromethoxy, —NH₂,

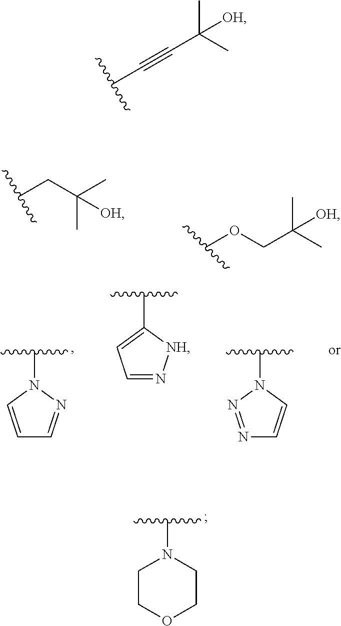

the phenyl fused 5-membered heterocycloalkyl can be substituted by ═O; the heteroaryl and the heterocycloalkyl each contain 1 to 3 heteroatoms selected from N, O or S; and R₆ is selected from methyl or —NH₂;
further preferably, R₁ and/or R₂ is selected from:

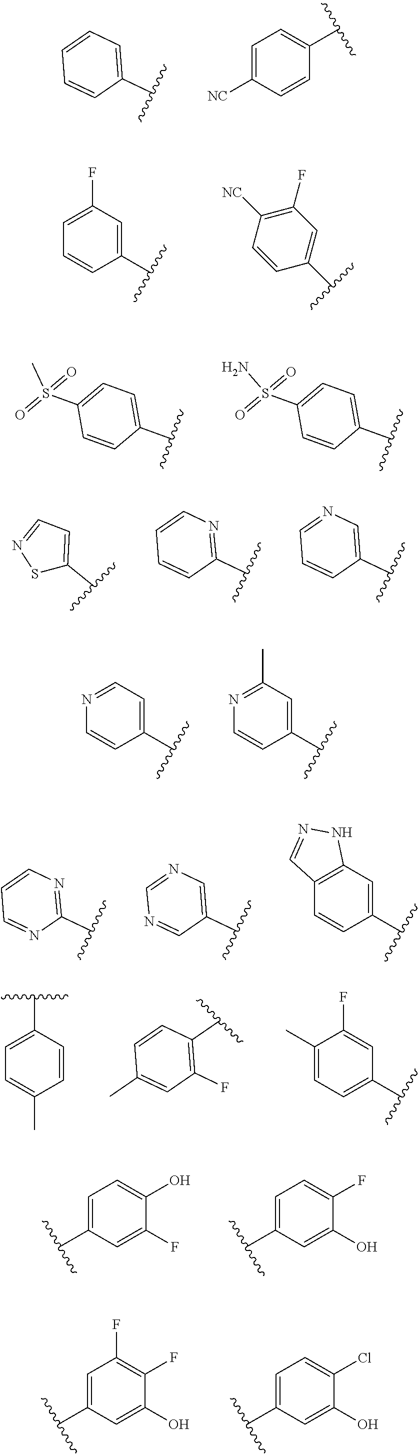

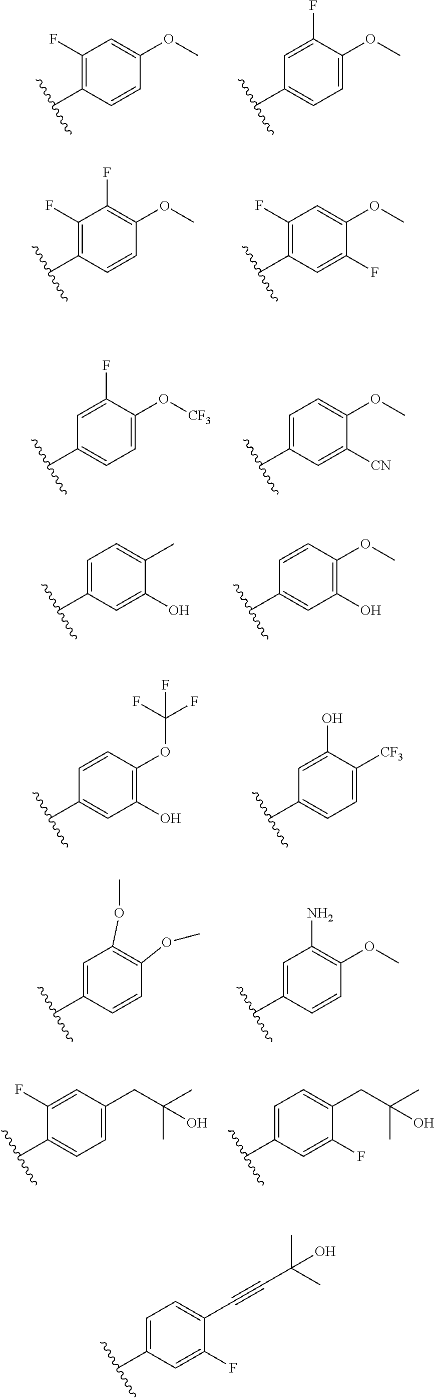

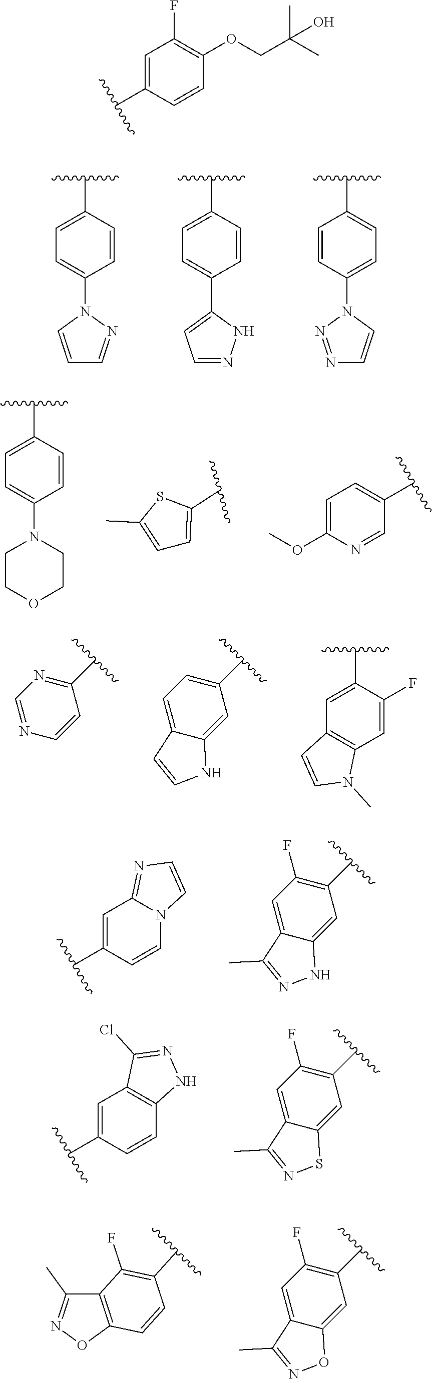

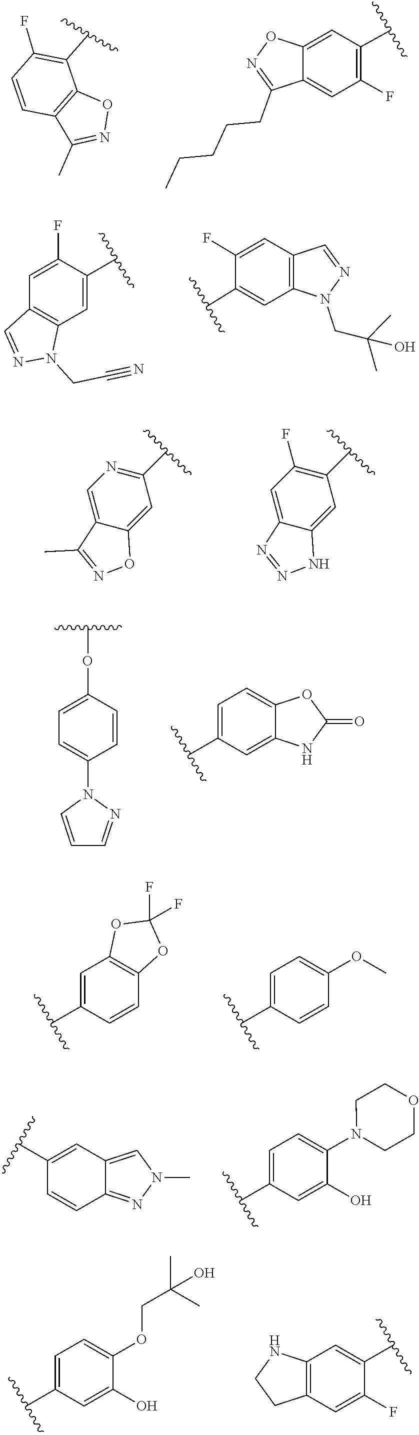

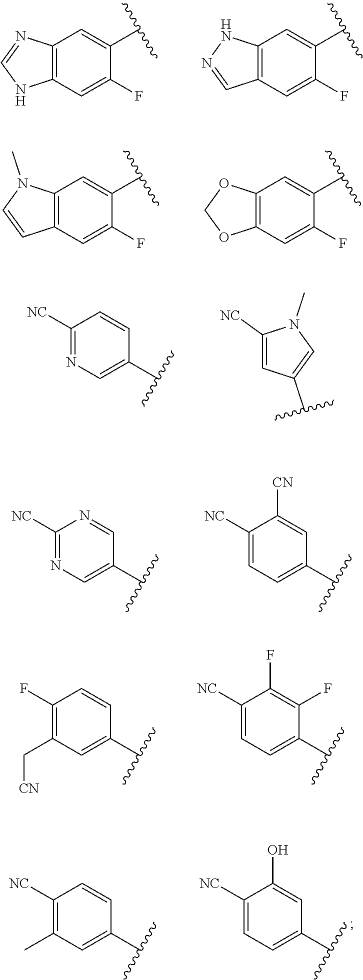

and
further preferably, R₁ and/or R₂ is selected from:

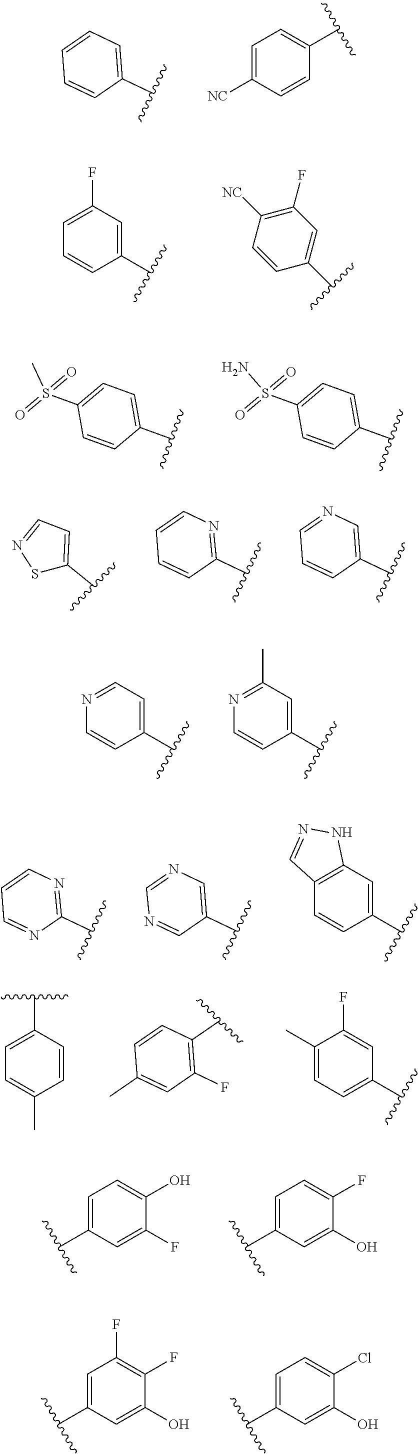

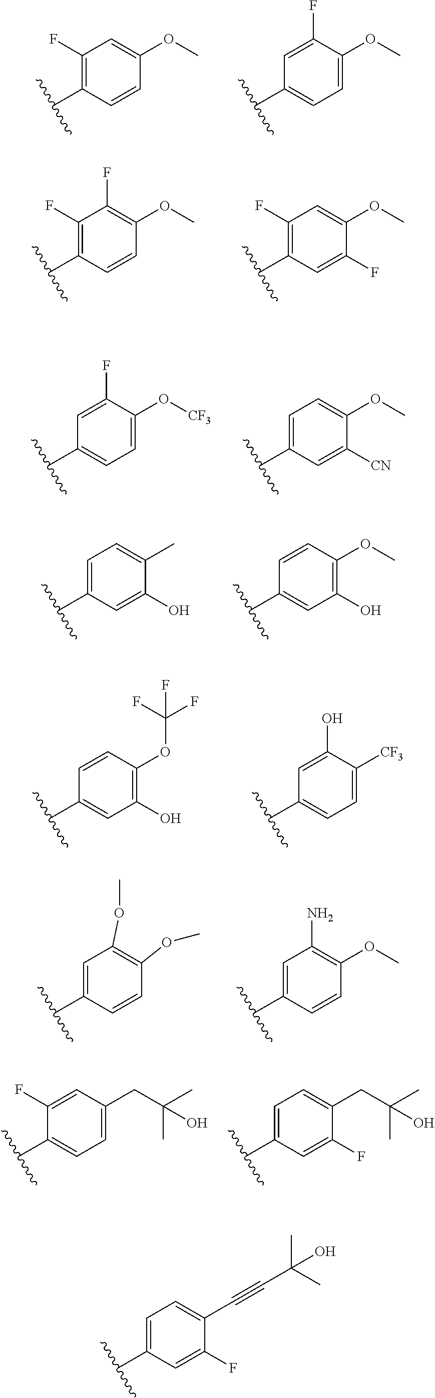

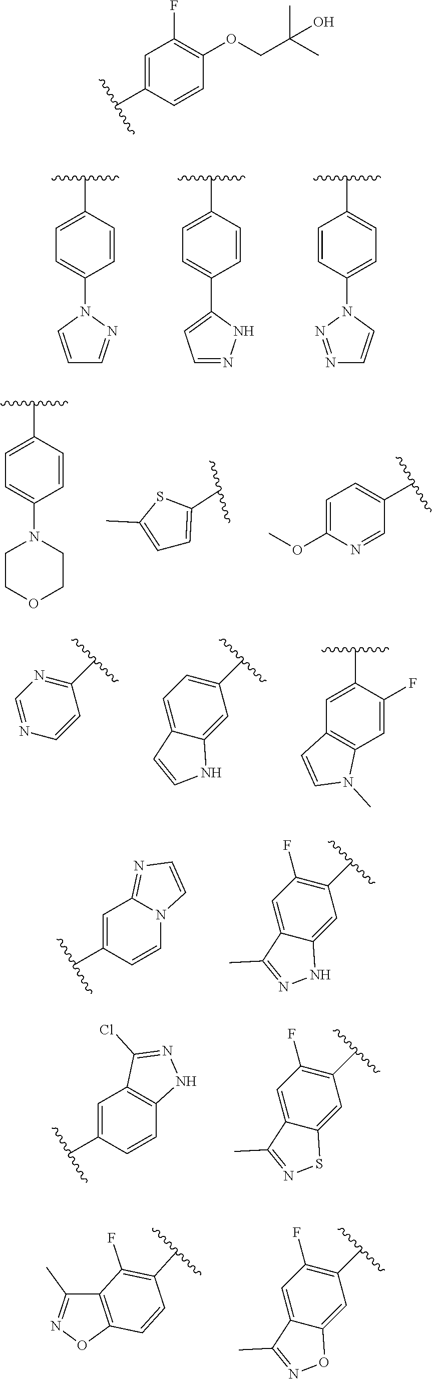

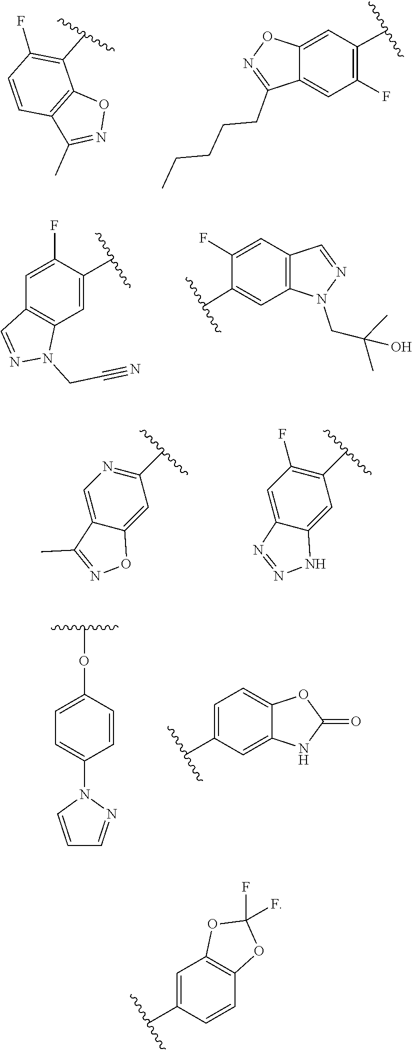

In an aspect of the present disclosure, wherein one of R₁ and R₂ is —NR₄R₅; R₄ and R₅ are each independently selected from H, C₆₋₁₀ alkyl, —NH₂ substituted C₁₋₆ alkyl, C₃₋₁₀ cycloalkyl, C₂₋₁₀ heterocycloalkyl, C₆₋₁₀ aryl or 5-10-membered heteroaryl; the C₆₋₁₀ aryl and the 5-10-membered heteroaryl are optionally substituted by 0 to 4 substituents selected from halogen or C₁₋₆ alkoxy; the C₂₋₁₀ heterocycloalkyl and the 5-10-membered heteroaryl contain 1 to 3 heteroatoms selected from N, O or S;
preferably, R₄ and R₅ are each independently selected from H, C₁₋₆ alkyl, —NH₂ substituted C₁₋₆ alkyl, 6-membered heterocycloalkyl or phenyl; the phenyl is optionally substituted by 0 to 2 substituents selected from halogen or C₁₋₆ alkoxy; the 6-membered heterocycloalkyl contains 1 to 2 heteroatoms selected from N or O; and further preferably, one of R₁ and R₂ is selected from

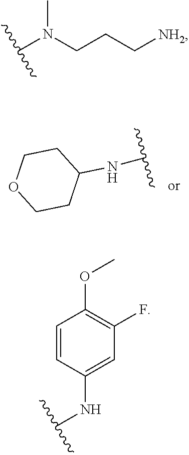

In an aspect of the present disclosure, wherein one of R₁ and R₂ is selected from C₃₋₁₀ cycloalkyl, C₂₋₁₀ heterocycloalkyl, C₃₋₁₀ cycloalkenyl or C₂₋₁₀ heterocycloalkenyl; the above groups are optionally substituted by 0 to 4 substitutents selected from halogen, —CN, hydroxy, C₁₋₆ alkyl, halogen substituted C₁₋₆ alkyl, C₁₋₆ alkoxy, halogen substituted C₁₋₆ alkoxy or ═O; preferably, one of R₁ and R₂ is selected from C₃₋₁₀ cycloalkyl or C₂₋₁₀ heterocycloalkenyl; the above groups are optionally substituted by 0 to 4 substituents selected from —CN, C₁₋₆ alkyl or ═O;
preferably, one of R₁ and R₂ is selected from

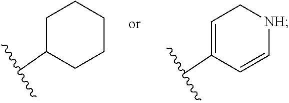 [SMILES: *C1CCCCC1 ; *C1=CCNC=C1]

the above groups are optionally substituted by 0 to 2 substituents selected from —CN, C₁₋₆ alkyl or ═O; and
further preferably, one of R₁ and R₂ is selected from

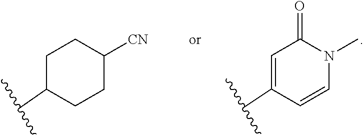

The present disclosure provides the following compounds:

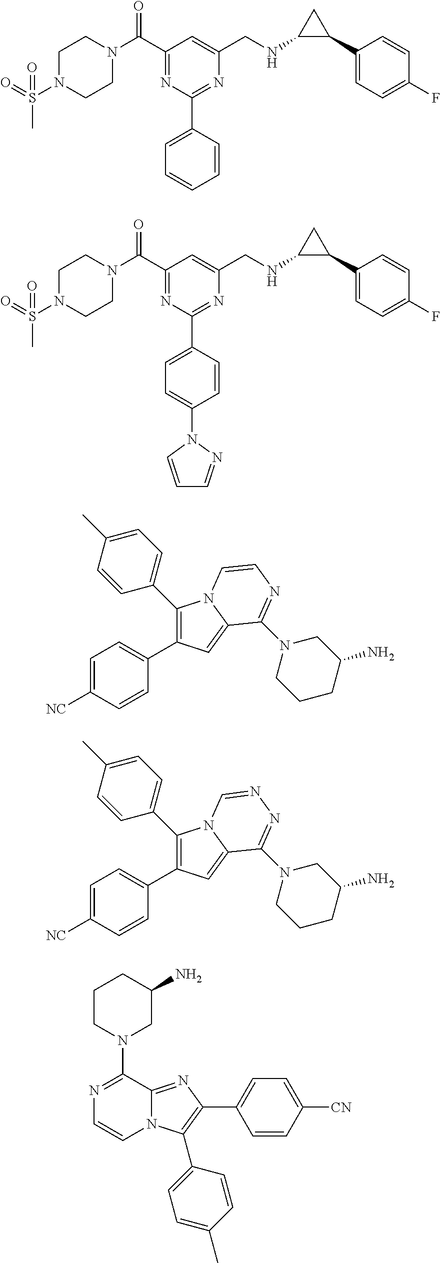

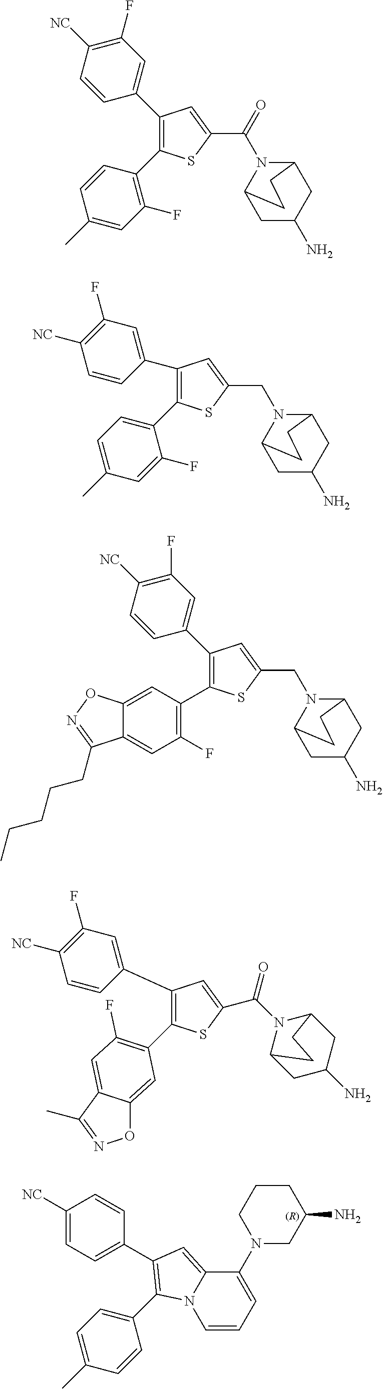

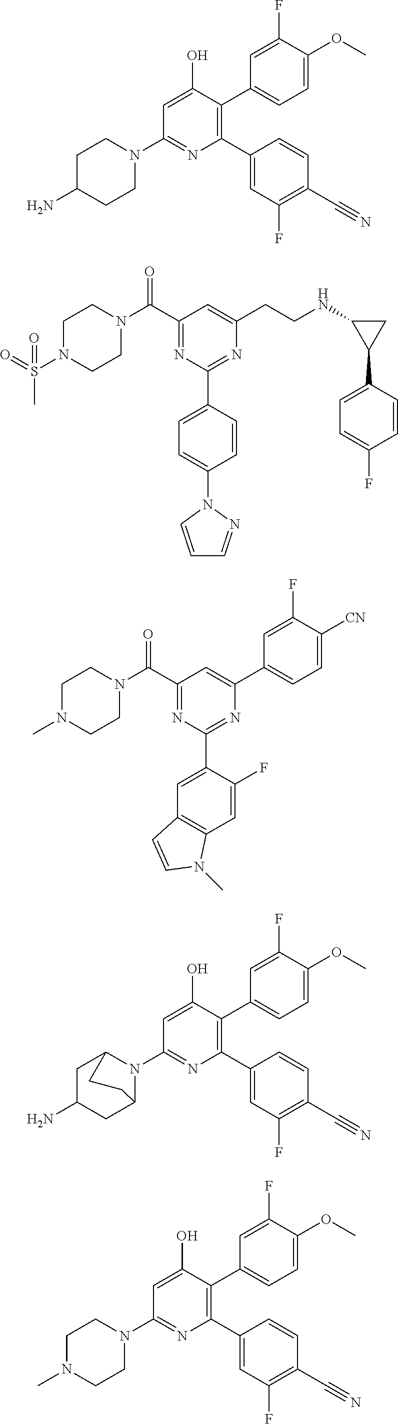

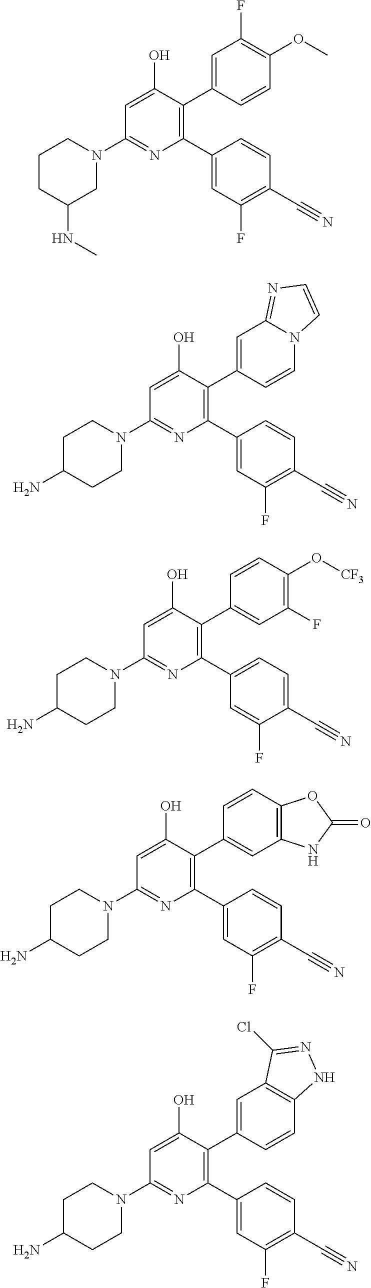

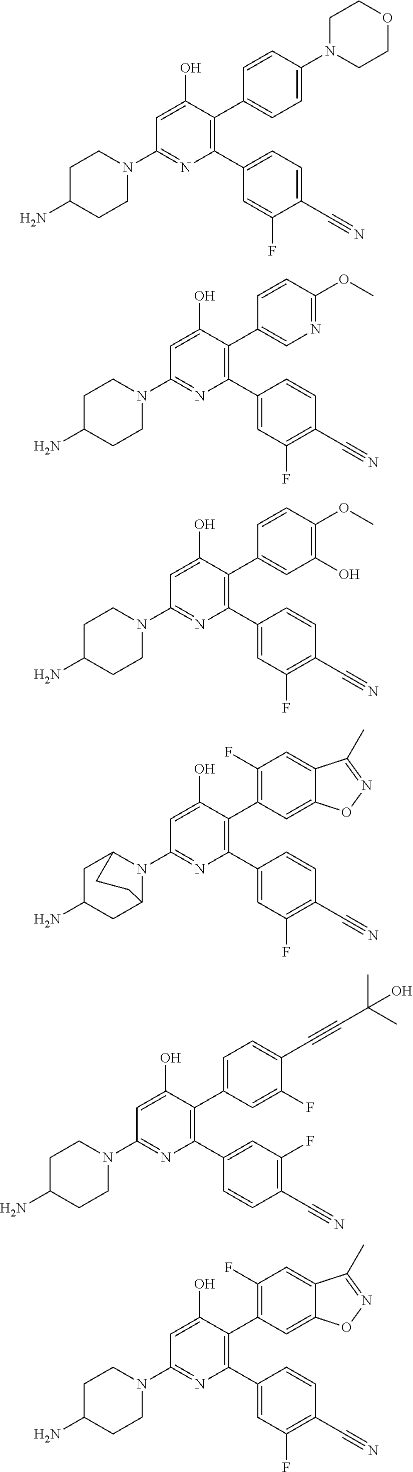

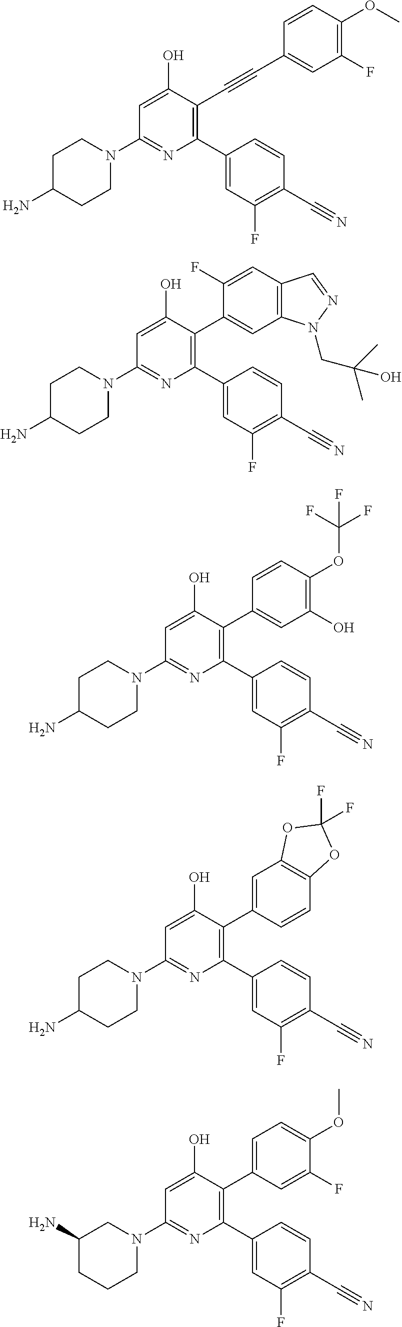

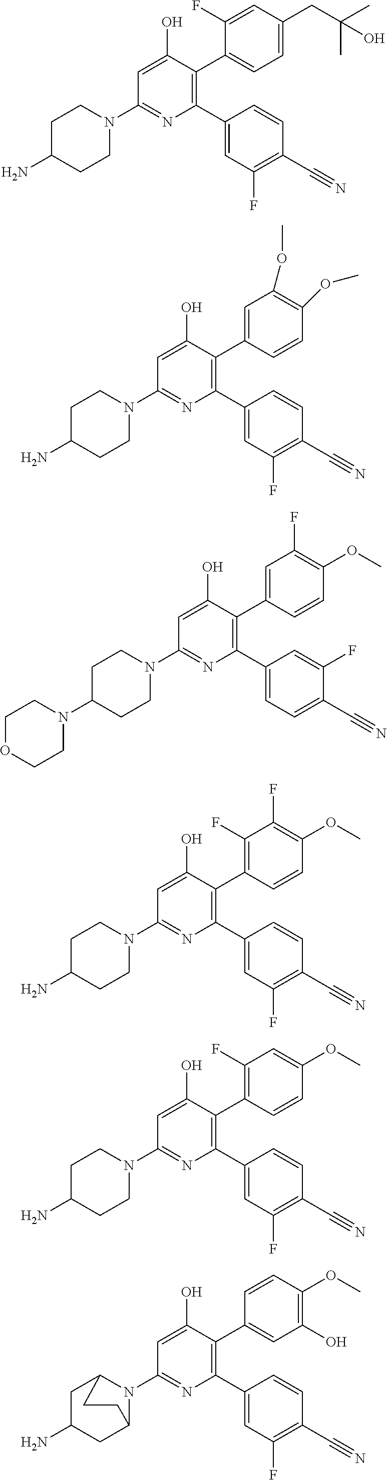

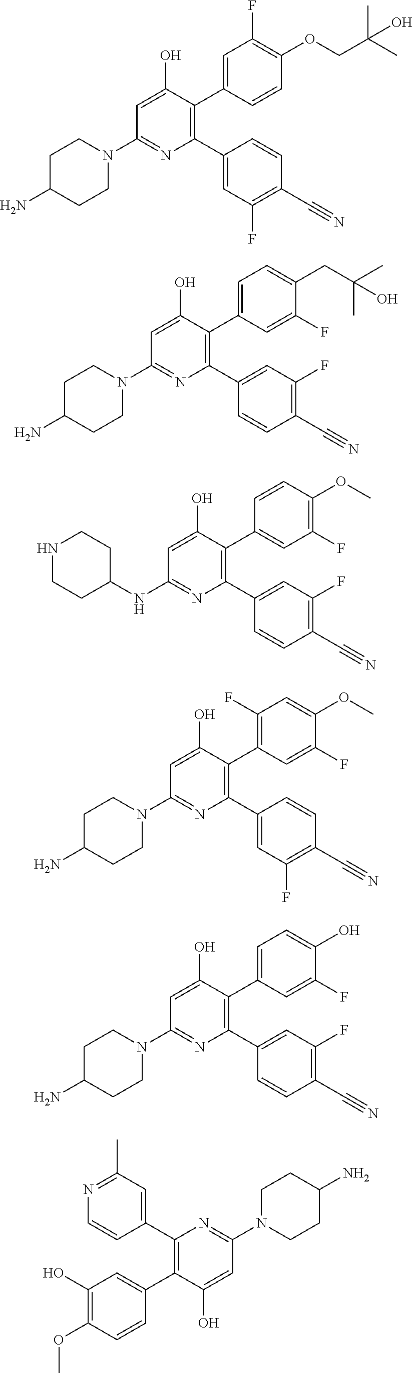

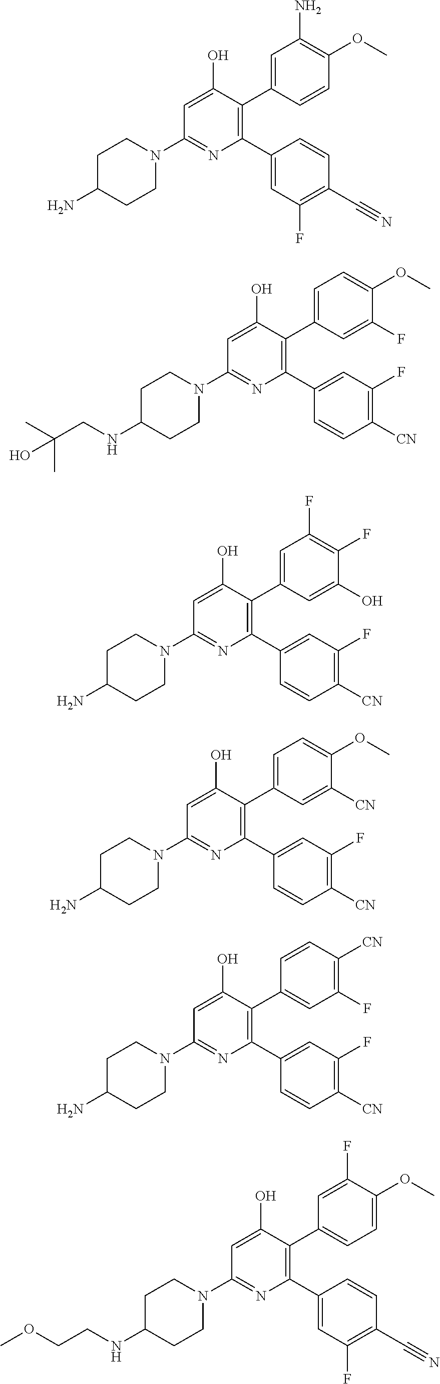

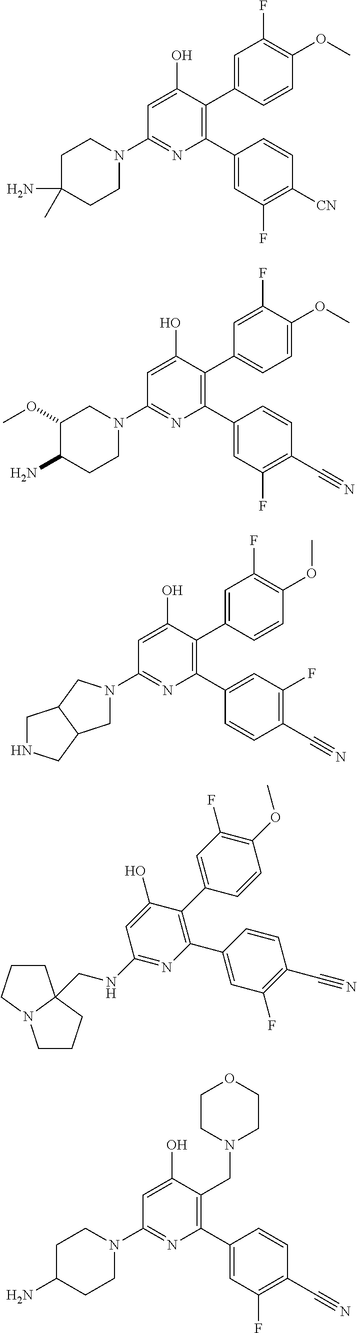

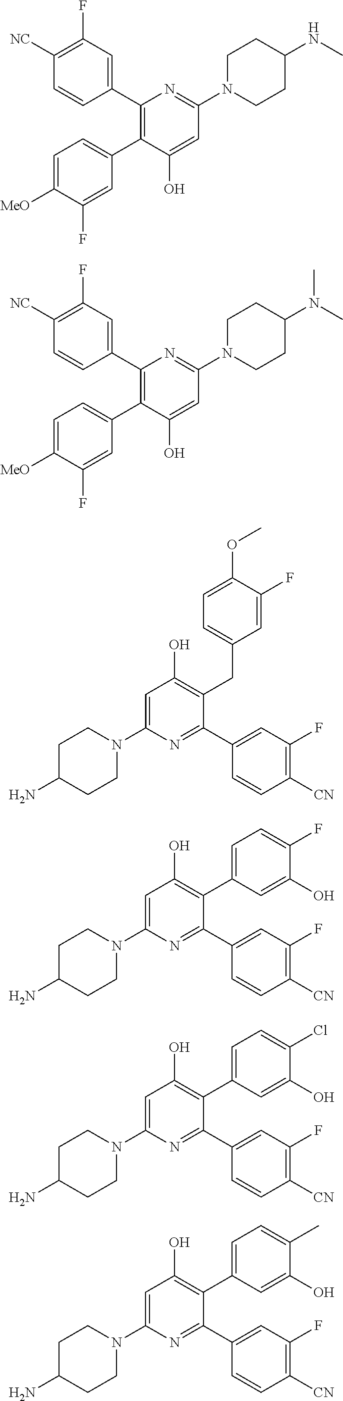

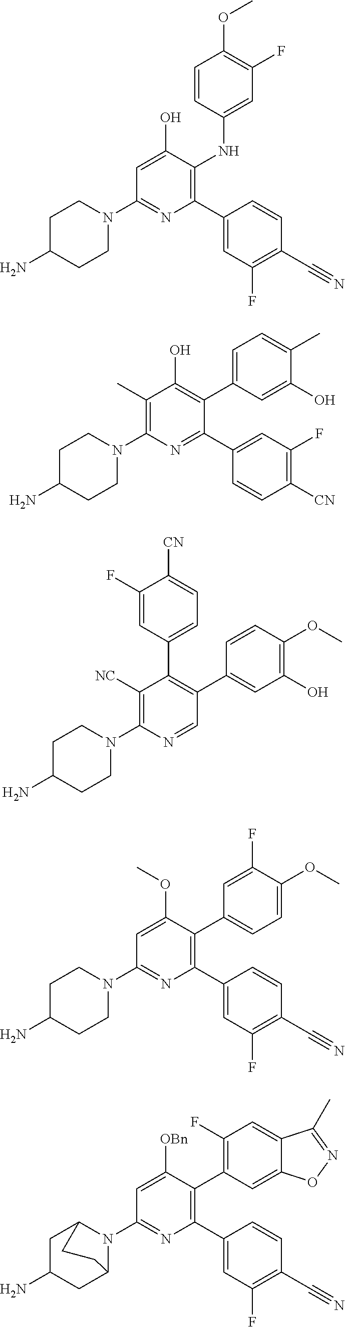

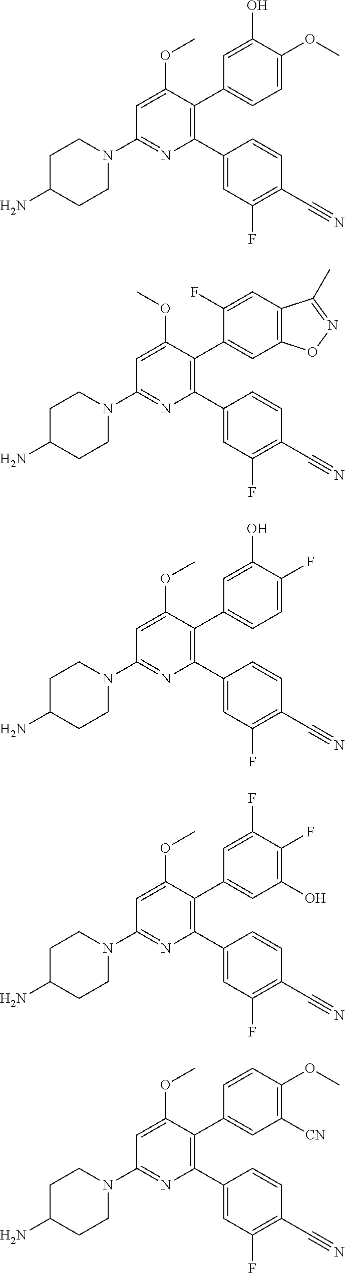

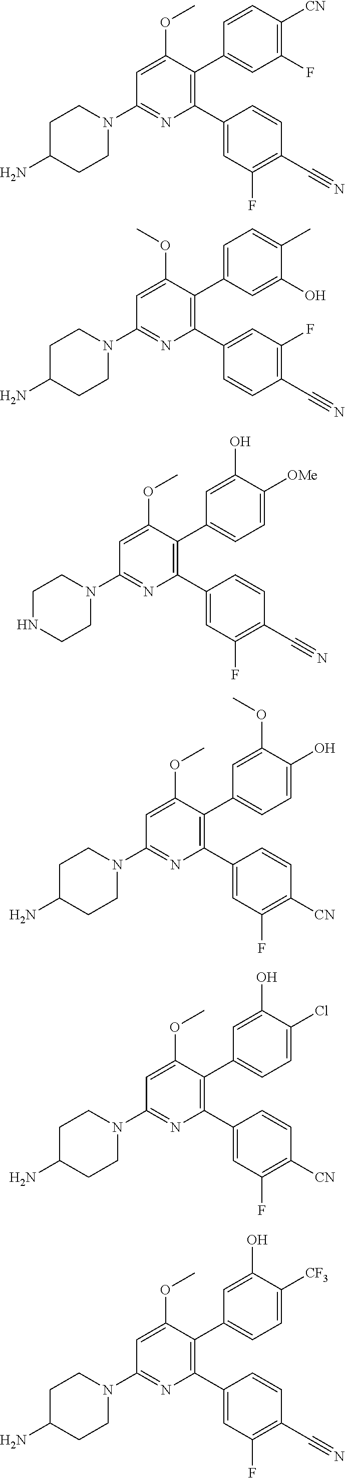

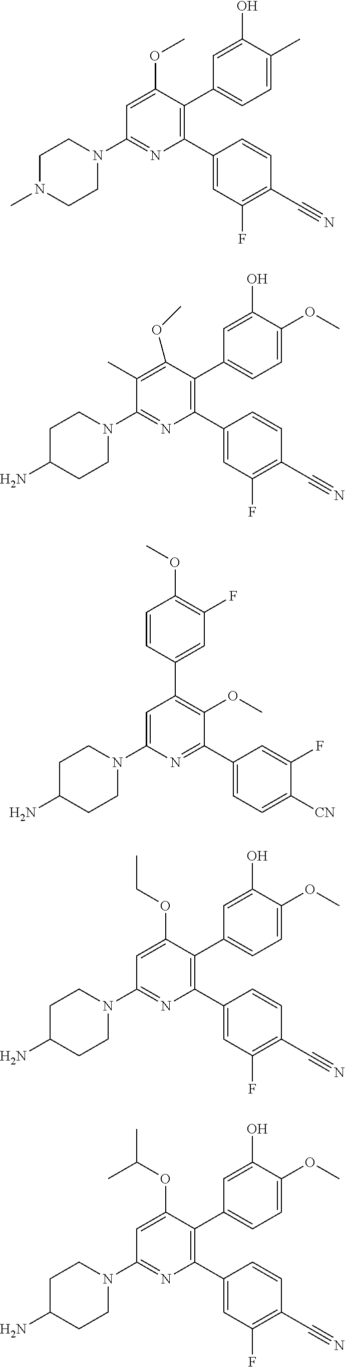

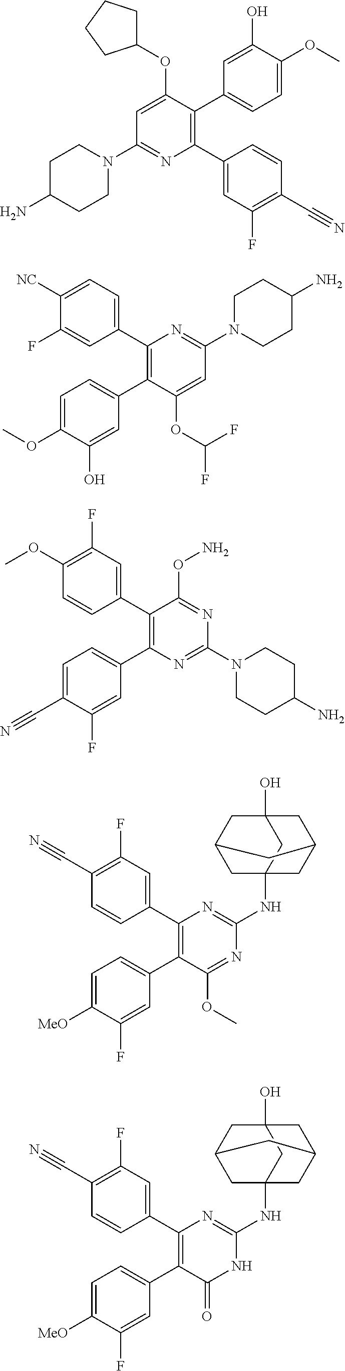

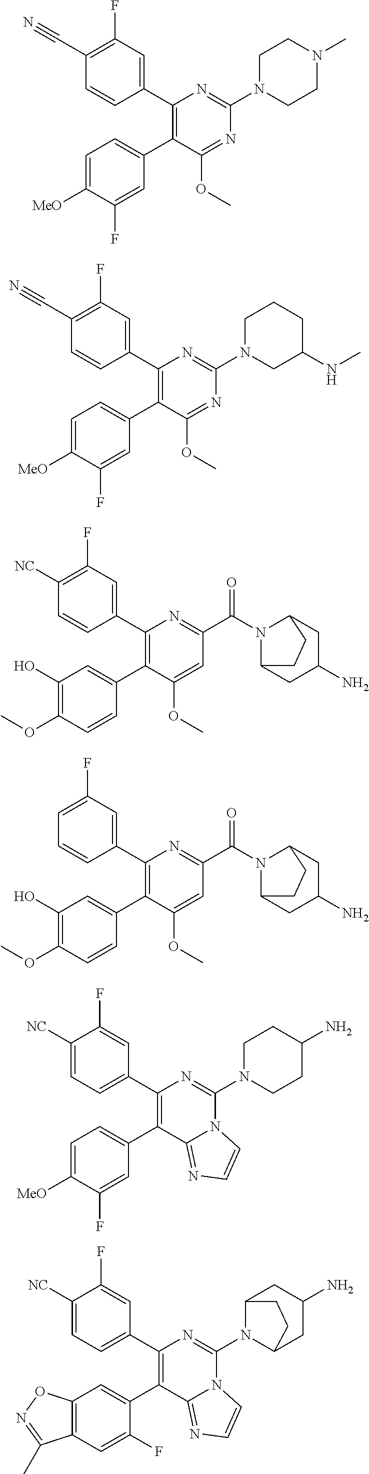

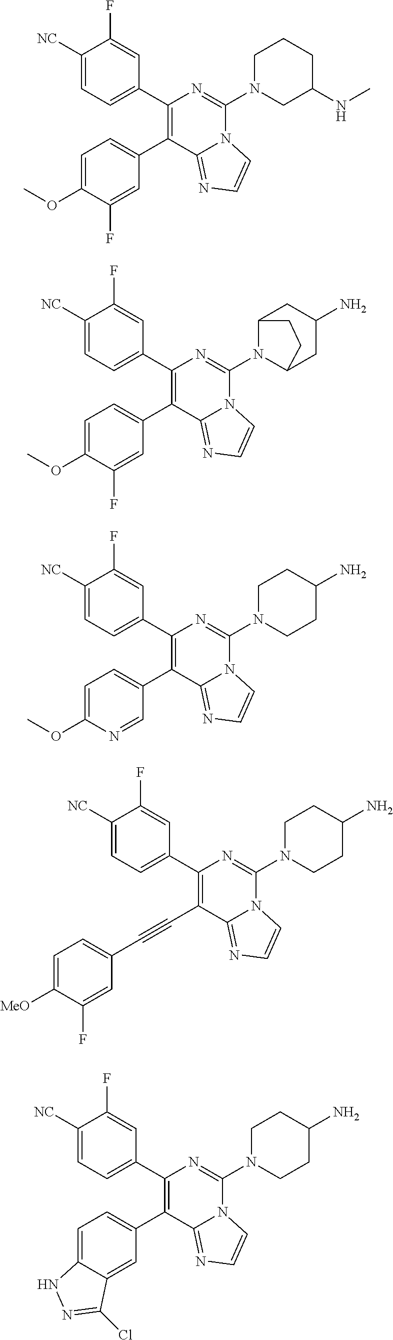

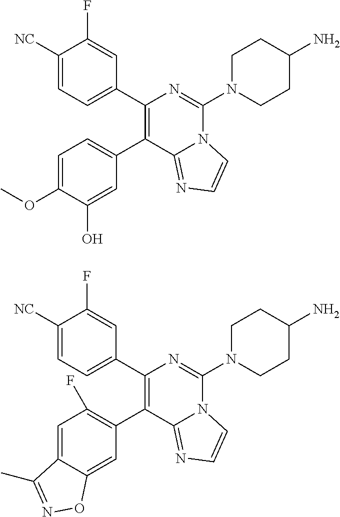

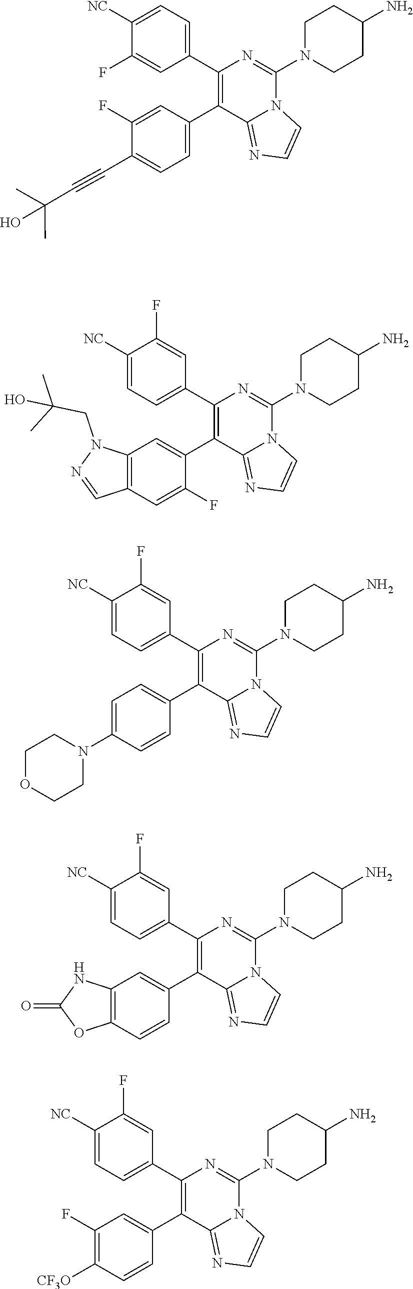

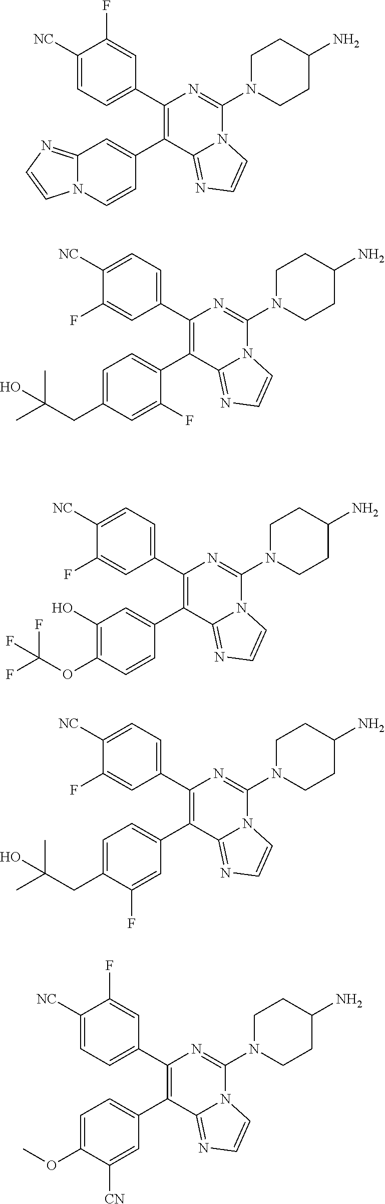 [SMILES: N#Cc1ccc(-c2nc(N3CCC(N)CC3)n3ccnc3c2-c2ccn3ccnc3c2)cc1F ; CC(C)(O)Cc1ccc(-c2c(-c3ccc(C#N)c(F)c3)nc(N3CCC(N)CC3)n3ccnc23)c(F)c1 ; N#Cc1ccc(-c2nc(N3CCC(N)CC3)n3ccnc3c2-c2ccc(OC(F)(F)F)c(O)c2)cc1F ; CC(C)(O)Cc1ccc(-c2c(-c3ccc(C#N)c(F)c3)nc(N3CCC(N)CC3)n3ccnc23)cc1F ; COc1ccc(-c2c(-c3ccc(C#N)c(F)c3)nc(N3CCC(N)CC3)n3ccnc23)cc1C#N]

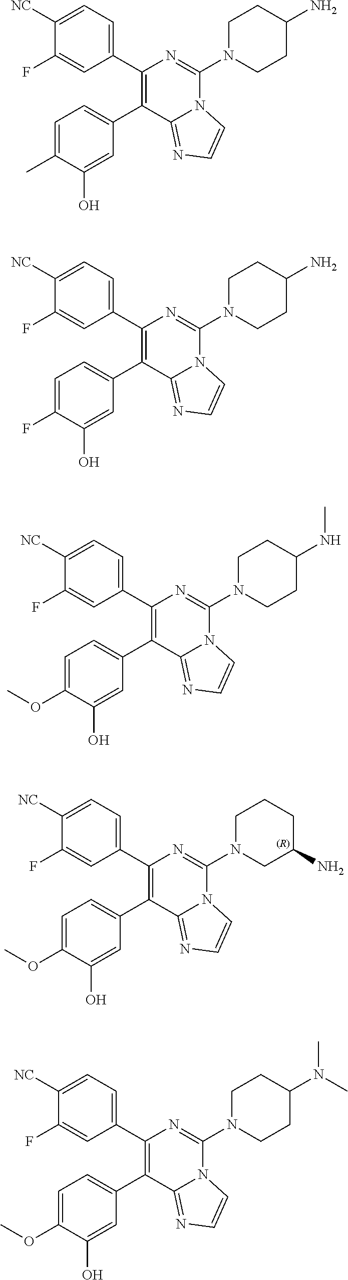

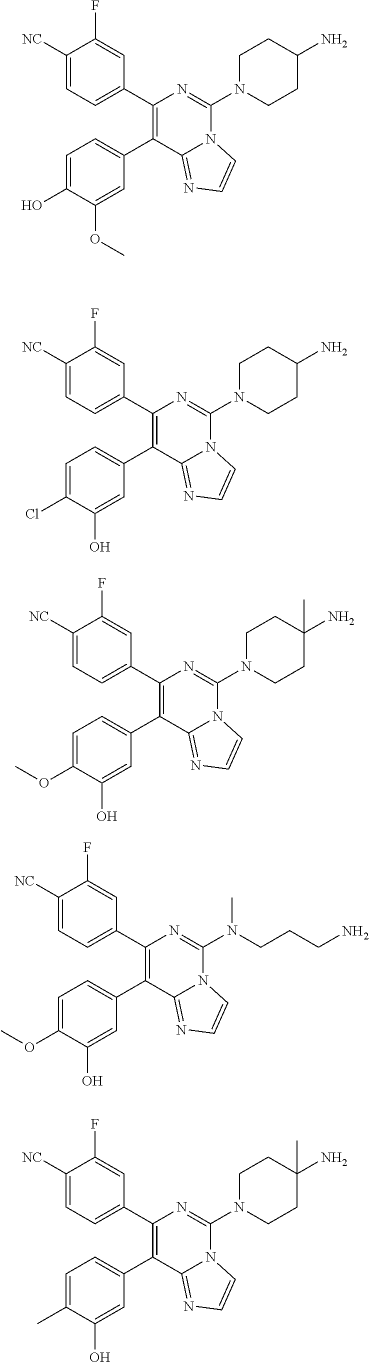

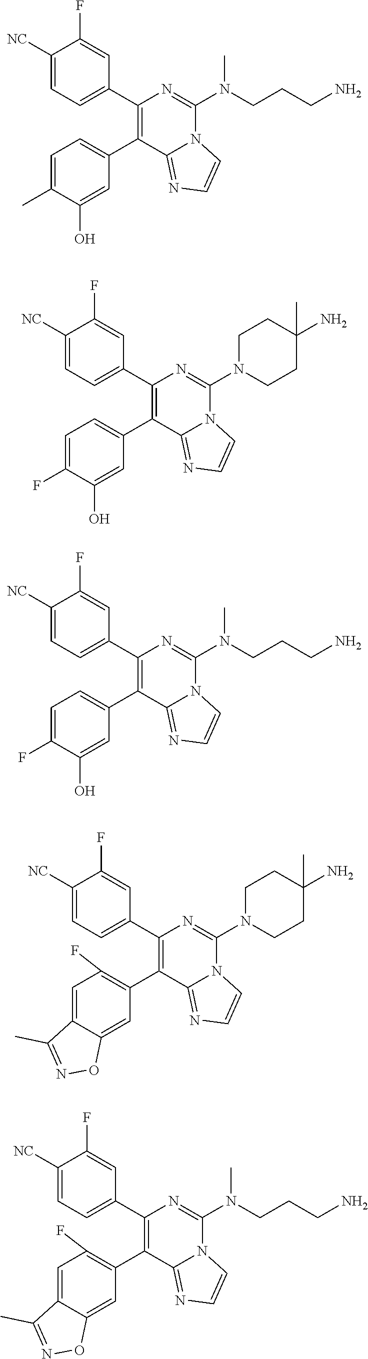

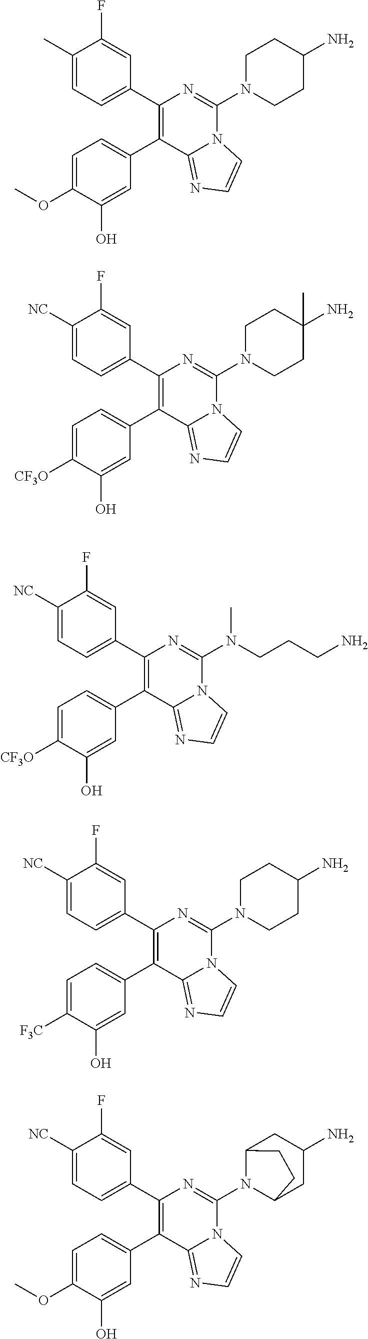

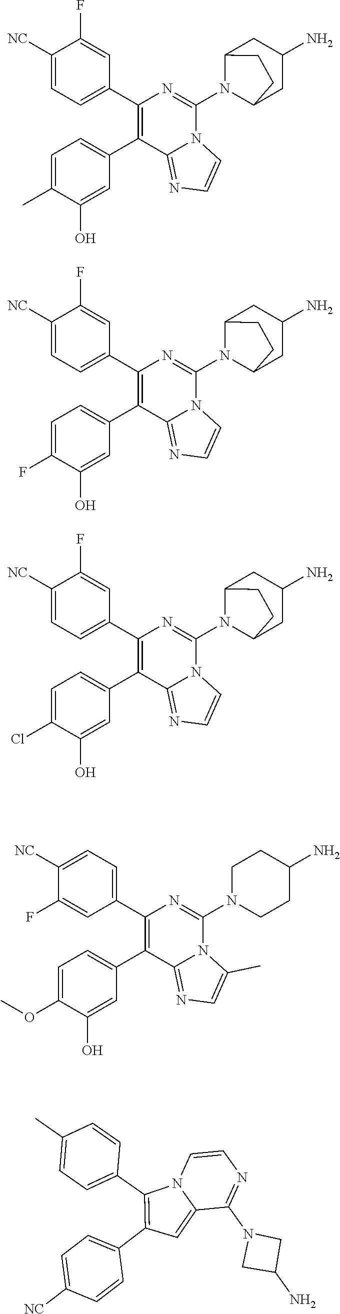

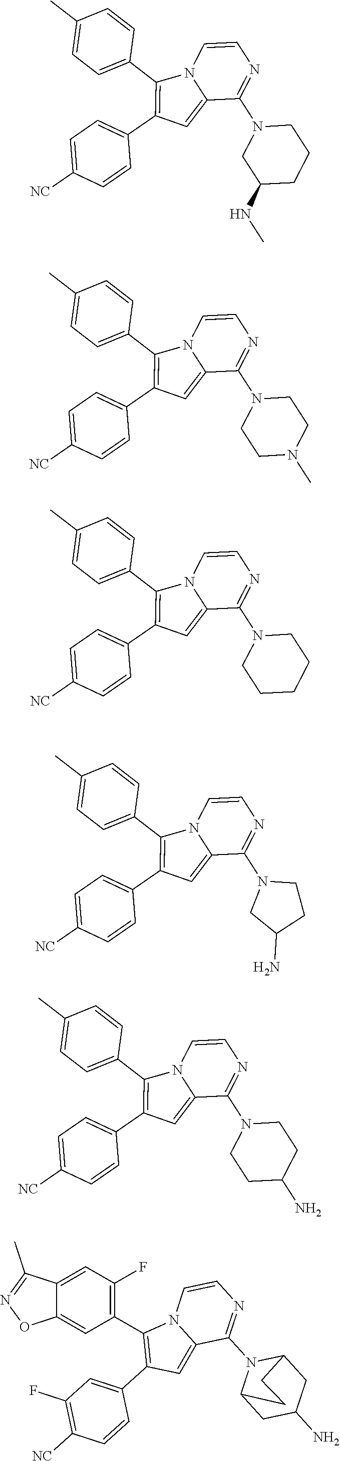 [SMILES: CN[C@@H]1CCCN(c2nccn3c(-c4ccc(C)cc4)c(-c4ccc(C#N)cc4)cc23)C1 ; Cc1ccc(-c2c(-c3ccc(C#N)cc3)cc3c(N4CCN(C)CC4)nccn23)cc1 ; Cc1ccc(-c2c(-c3ccc(C#N)cc3)cc3c(N4CCCCC4)nccn23)cc1 ; Cc1ccc(-c2c(-c3ccc(C#N)cc3)cc3c(N4CCC(N)C4)nccn23)cc1 ; Cc1ccc(-c2c(-c3ccc(C#N)cc3)cc3c(N4CCC(N)CC4)nccn23)cc1 ; Cc1noc2cc(-c3c(-c4ccc(C#N)c(F)c4)cc4c(N5C6CCC5CC(N)C6)nccn34)c(F)cc12]

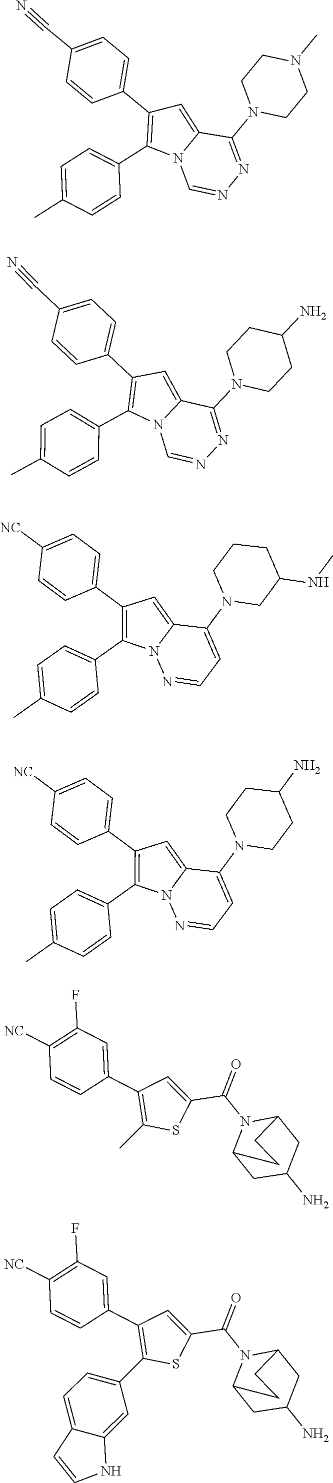

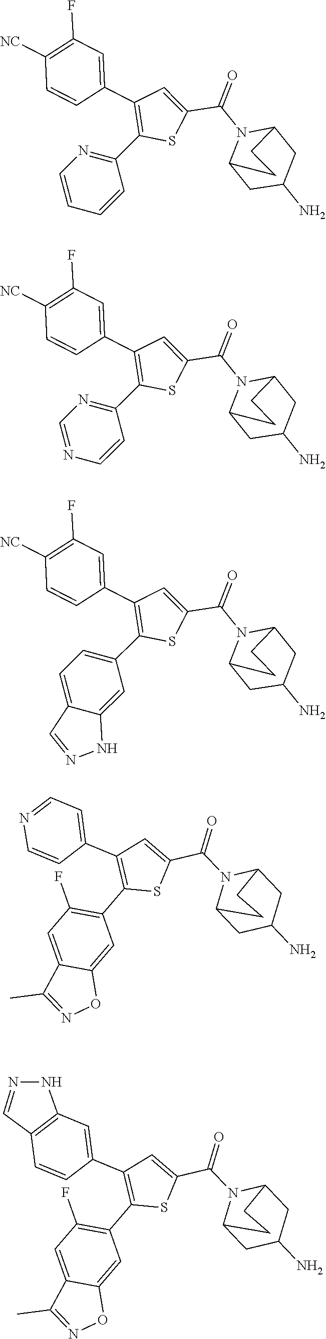

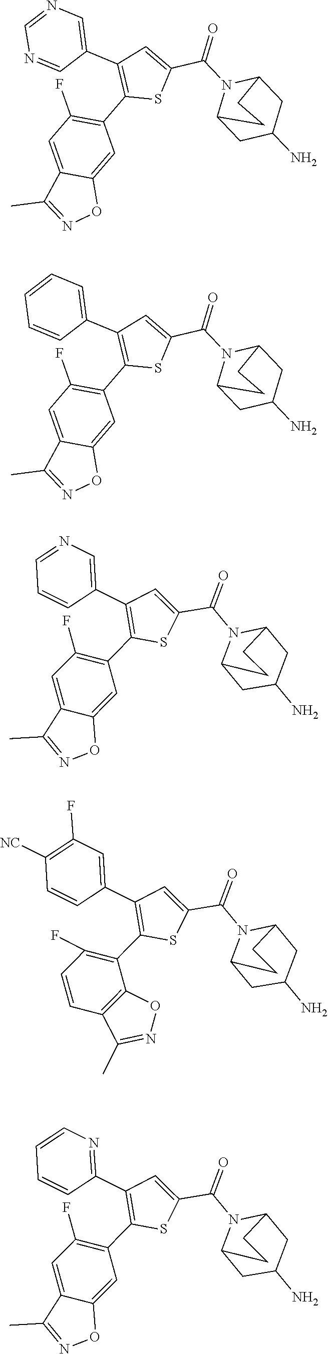

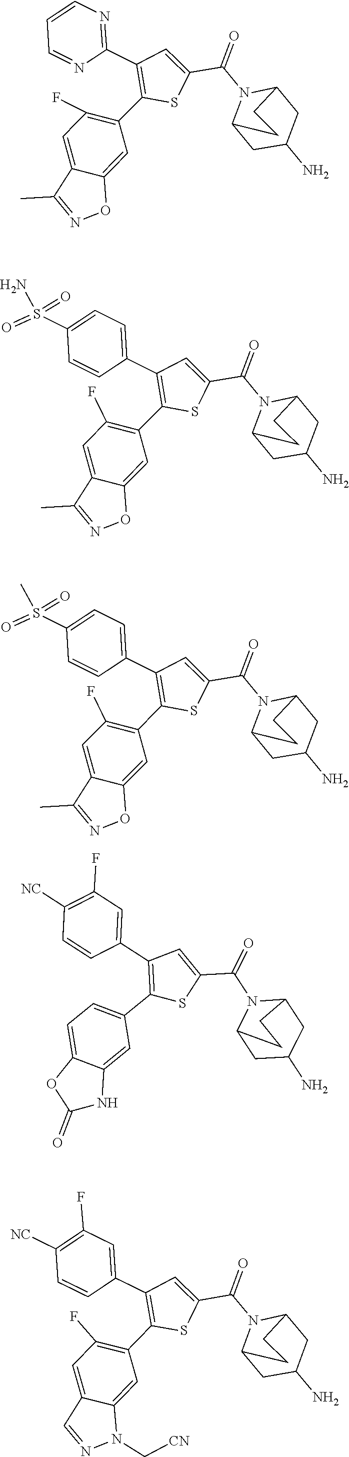

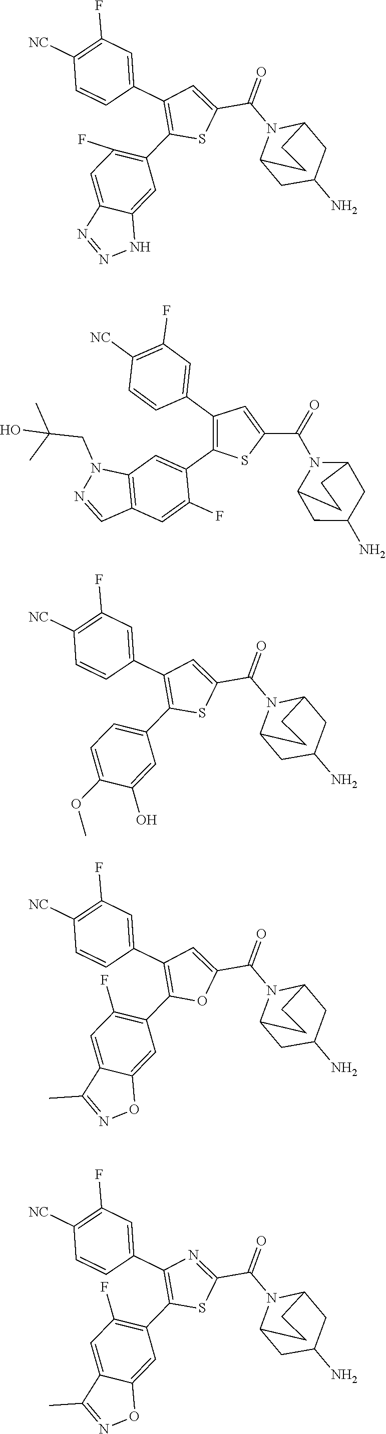

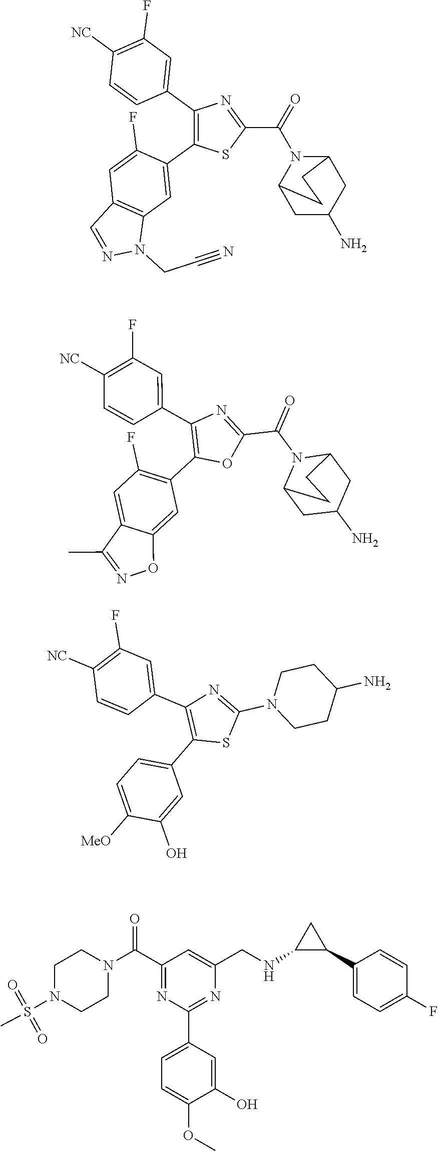

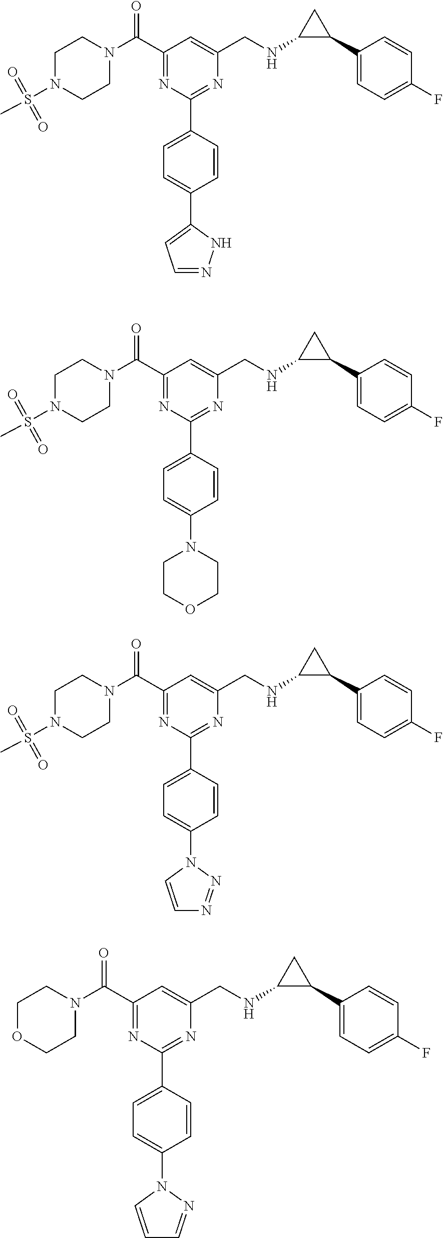 [SMILES: CS(=O)(=O)N1CCN(C(=O)c2cc(CN[C@@H]3C[C@H]3c3ccc(F)cc3)nc(-c3ccc(-c4ccn[nH]4)cc3)n2)CC1 ; CS(=O)(=O)N1CCN(C(=O)c2cc(CN[C@@H]3C[C@H]3c3ccc(F)cc3)nc(-c3ccc(N4CCOCC4)cc3)n2)CC1 ; CS(=O)(=O)N1CCN(C(=O)c2cc(CN[C@@H]3C[C@H]3c3ccc(F)cc3)nc(-c3ccc(-n4ccnn4)cc3)n2)CC1 ; O=C(c1cc(CN[C@@H]2C[C@H]2c2ccc(F)cc2)nc(-c2ccc(-n3cccn3)cc2)n1)N1CCOCC1]

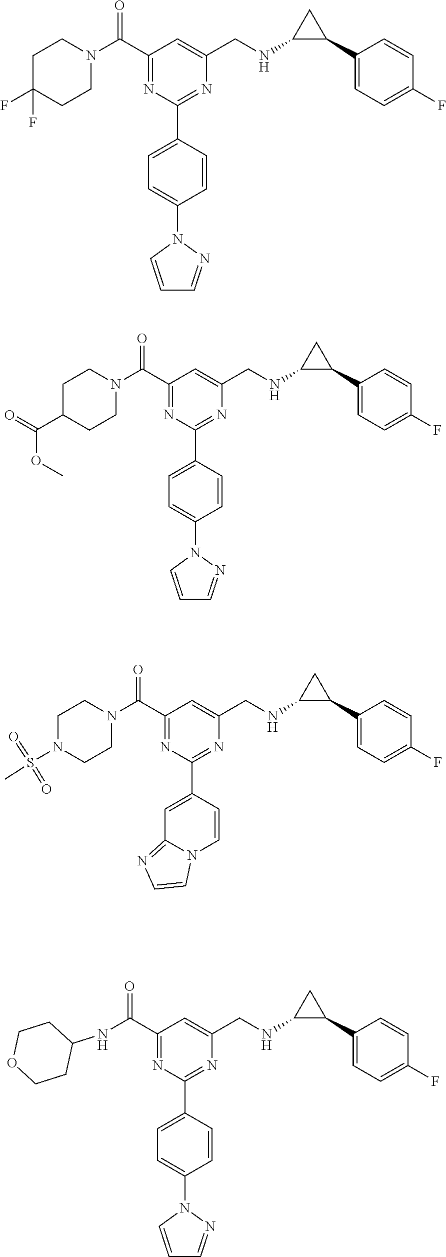 [SMILES: O=C(c1cc(CN[C@@H]2C[C@H]2c2ccc(F)cc2)nc(-c2ccc(-n3cccn3)cc2)n1)N1CCC(F)(F)CC1 ; COC(=O)C1CCN(C(=O)c2cc(CN[C@@H]3C[C@H]3c3ccc(F)cc3)nc(-c3ccc(-n4cccn4)cc3)n2)CC1 ; CS(=O)(=O)N1CCN(C(=O)c2cc(CN[C@@H]3C[C@H]3c3ccc(F)cc3)nc(-c3ccn4ccnc4c3)n2)CC1 ; O=C(NC1CCOCC1)c1cc(CN[C@@H]2C[C@H]2c2ccc(F)cc2)nc(-c2ccc(-n3cccn3)cc2)n1]

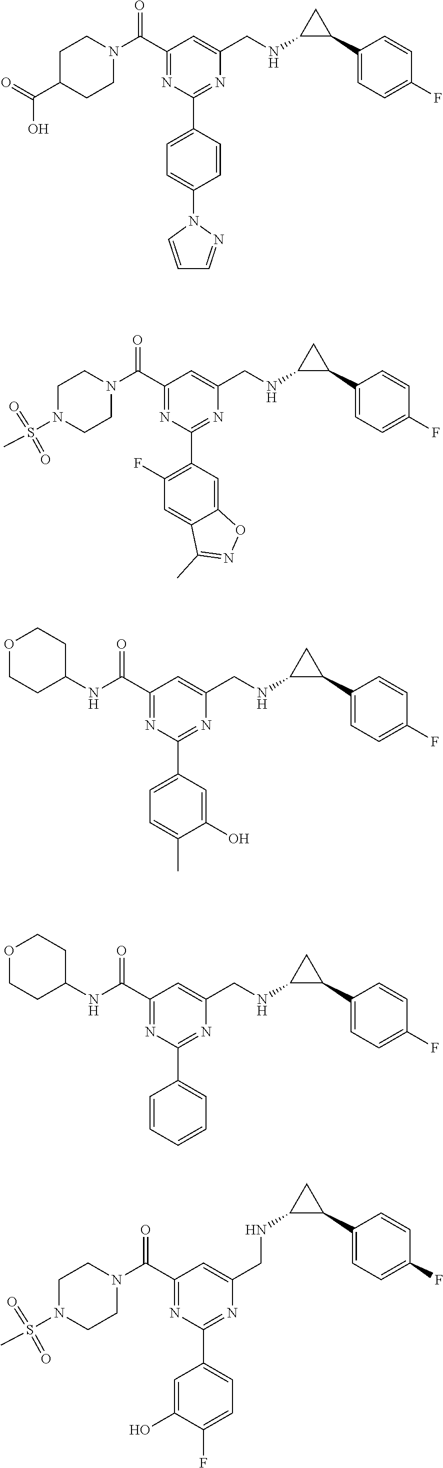

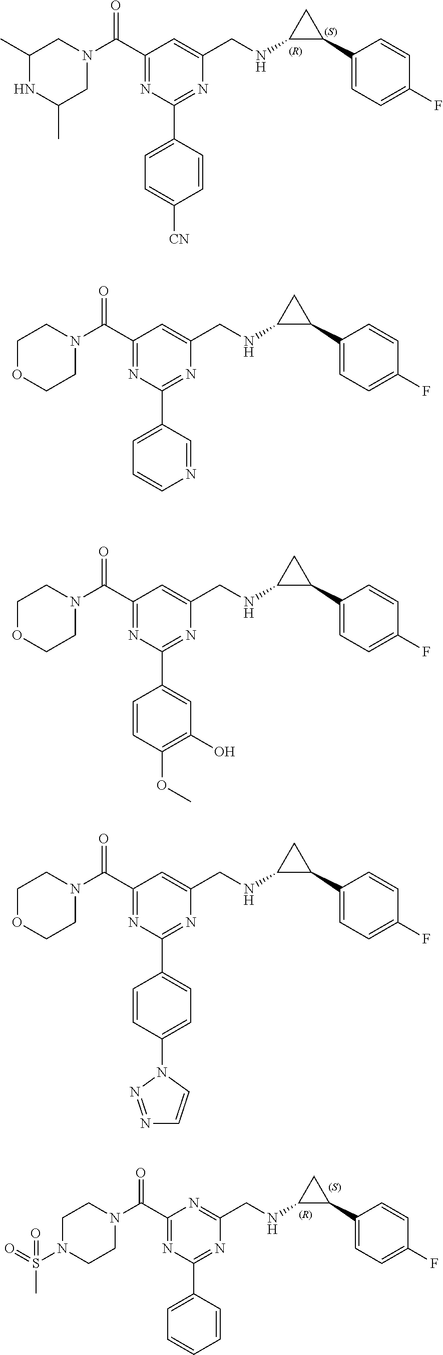

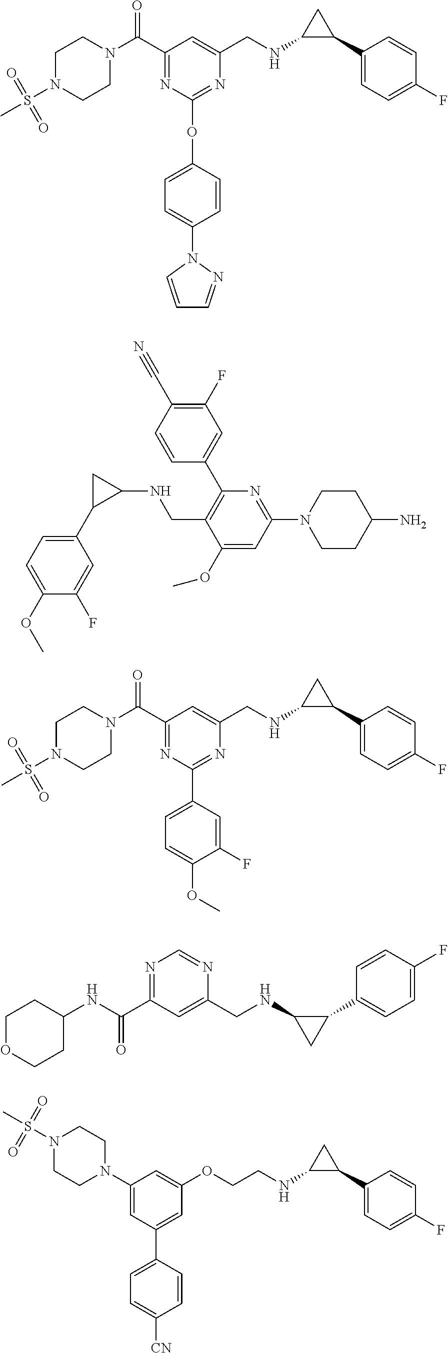

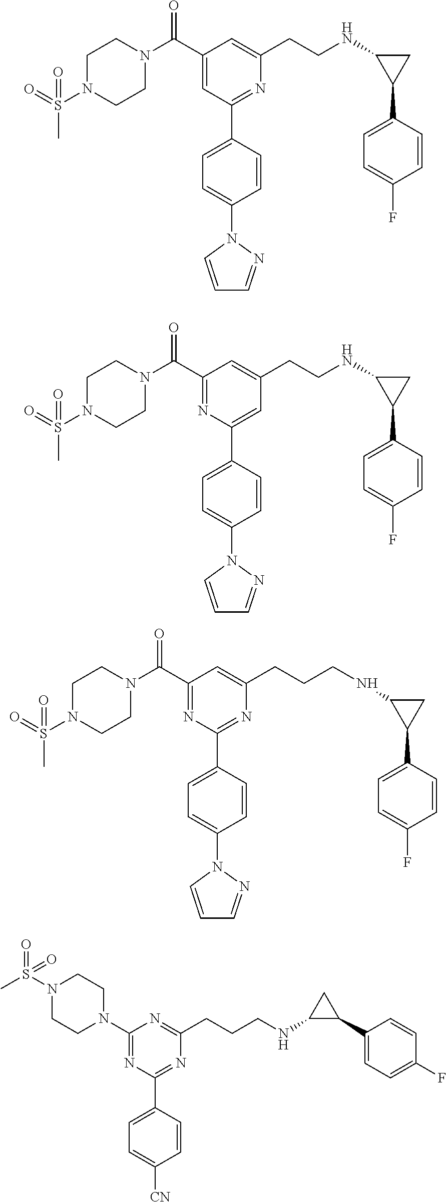

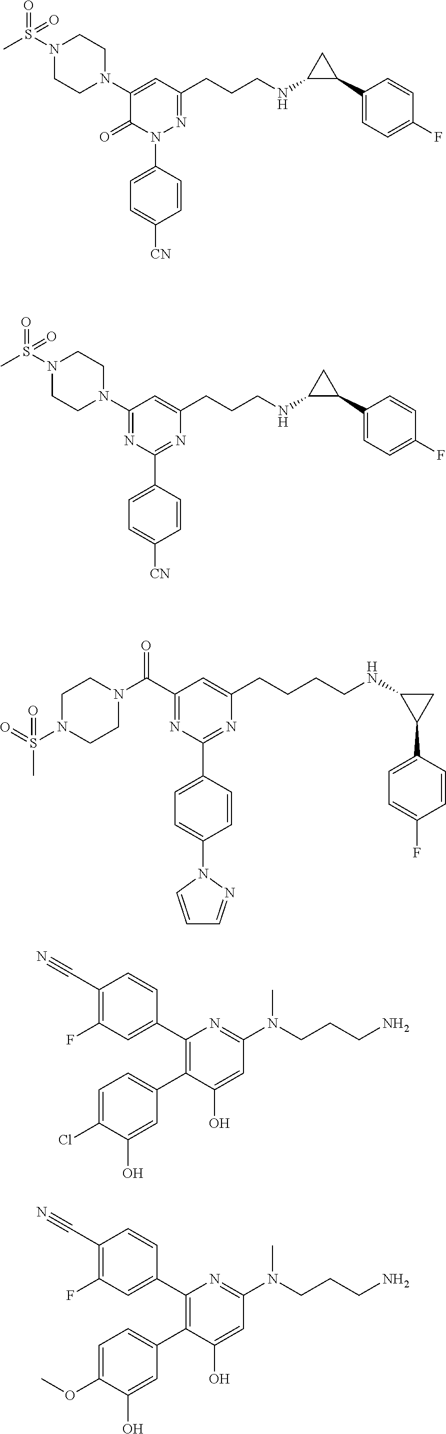 [SMILES: CS(=O)(=O)N1CCN(c2cc(CCCN[C@@H]3C[C@H]3c3ccc(F)cc3)nn(-c3ccc(C#N)cc3)c2=O)CC1 ; CS(=O)(=O)N1CCN(c2cc(CCCN[C@@H]3C[C@H]3c3ccc(F)cc3)nc(-c3ccc(C#N)cc3)n2)CC1 ; CS(=O)(=O)N1CCN(C(=O)c2cc(CCCCN[C@@H]3C[C@H]3c3ccc(F)cc3)nc(-c3ccc(-n4cccn4)cc3)n2)CC1 ; CN(CCCN)c1cc(O)c(-c2ccc(Cl)c(O)c2)c(-c2ccc(C#N)c(F)c2)n1 ; COc1ccc(-c2c(O)cc(N(C)CCCN)nc2-c2ccc(C#N)c(F)c2)cc1O]

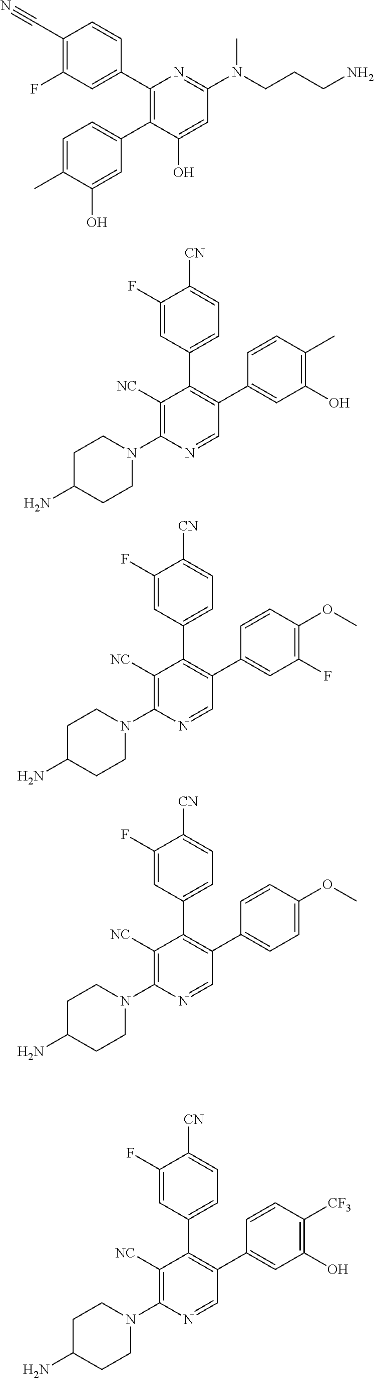 [SMILES: Cc1ccc(-c2c(O)cc(N(C)CCCN)nc2-c2ccc(C#N)c(F)c2)cc1O ; Cc1ccc(-c2cnc(N3CCC(N)CC3)c(C#N)c2-c2ccc(C#N)c(F)c2)cc1O ; COc1ccc(-c2cnc(N3CCC(N)CC3)c(C#N)c2-c2ccc(C#N)c(F)c2)cc1F ; COc1ccc(-c2cnc(N3CCC(N)CC3)c(C#N)c2-c2ccc(C#N)c(F)c2)cc1 ; N#Cc1ccc(-c2c(-c3ccc(C(F)(F)F)c(O)c3)cnc(N3CCC(N)CC3)c2C#N)cc1F]

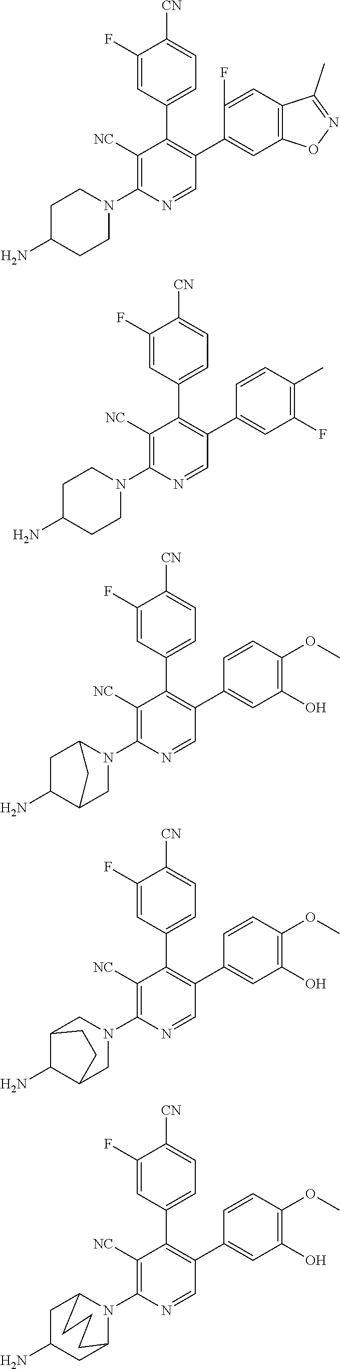 [SMILES: Cc1noc2cc(-c3cnc(N4CCC(N)CC4)c(C#N)c3-c3ccc(C#N)c(F)c3)c(F)cc12 ; Cc1ccc(-c2cnc(N3CCC(N)CC3)c(C#N)c2-c2ccc(C#N)c(F)c2)cc1F ; COc1ccc(-c2cnc(N3CC4CC3CC4N)c(C#N)c2-c2ccc(C#N)c(F)c2)cc1O ; COc1ccc(-c2cnc(N3CC4CCC(C3)C4N)c(C#N)c2-c2ccc(C#N)c(F)c2)cc1O ; COc1ccc(-c2cnc(N3C4CCCC3CC(N)C4)c(C#N)c2-c2ccc(C#N)c(F)c2)cc1O]

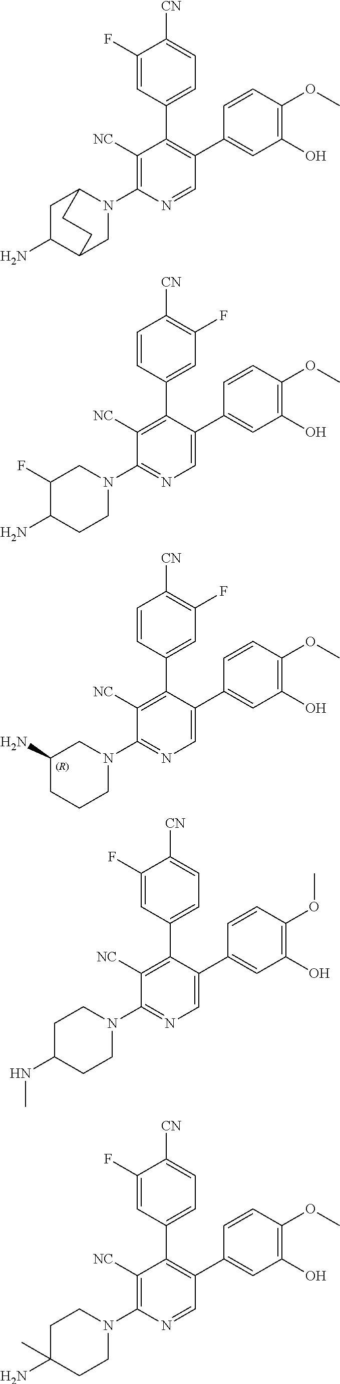

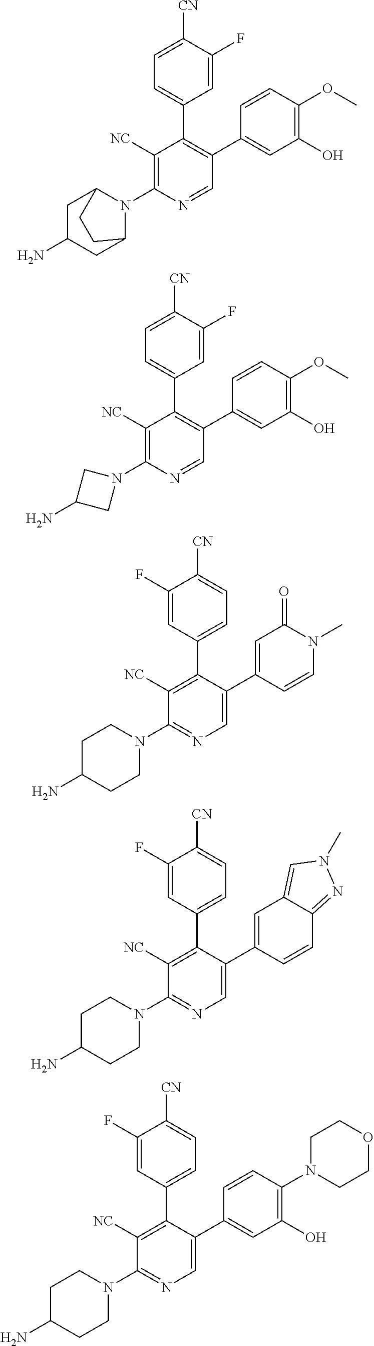 [SMILES: COc1ccc(-c2cnc(N3C4CCC3CC(N)C4)c(C#N)c2-c2ccc(C#N)c(F)c2)cc1O ; COc1ccc(-c2cnc(N3CC(N)C3)c(C#N)c2-c2ccc(C#N)c(F)c2)cc1O ; Cn1ccc(-c2cnc(N3CCC(N)CC3)c(C#N)c2-c2ccc(C#N)c(F)c2)cc1=O ; Cn1cc2cc(-c3cnc(N4CCC(N)CC4)c(C#N)c3-c3ccc(C#N)c(F)c3)ccc2n1 ; N#Cc1ccc(-c2c(-c3ccc(N4CCOCC4)c(O)c3)cnc(N3CCC(N)CC3)c2C#N)cc1F]

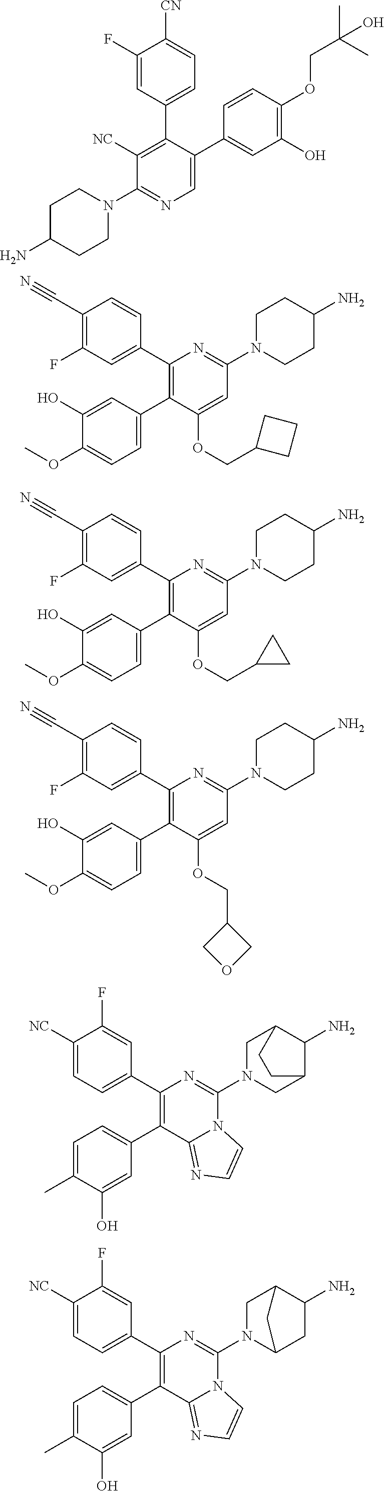

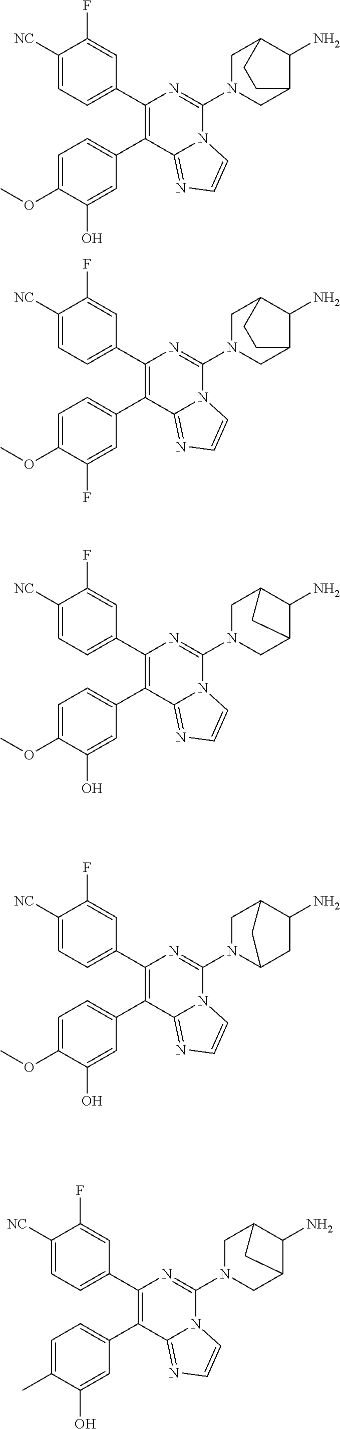

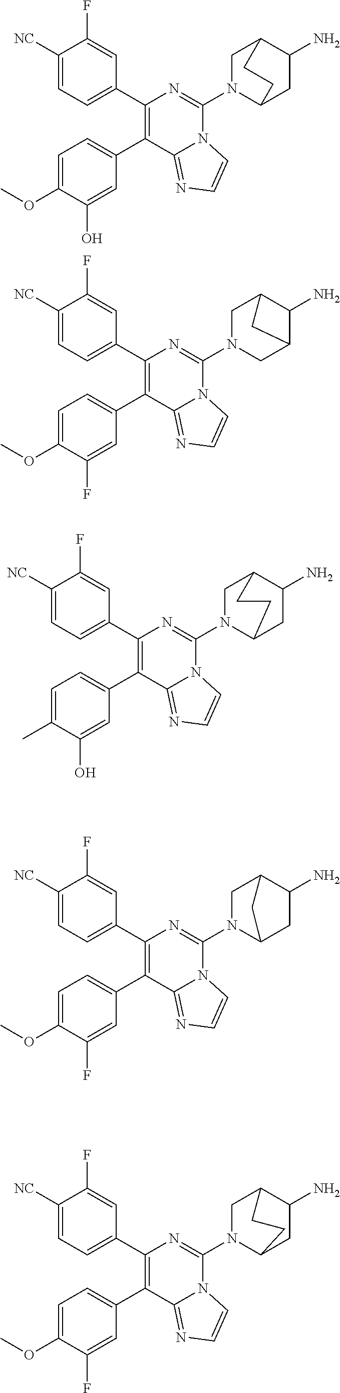

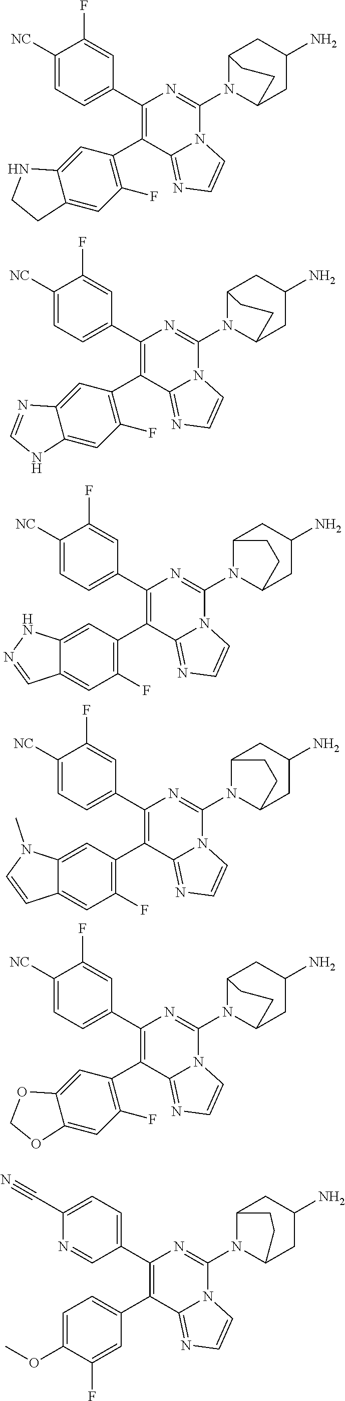

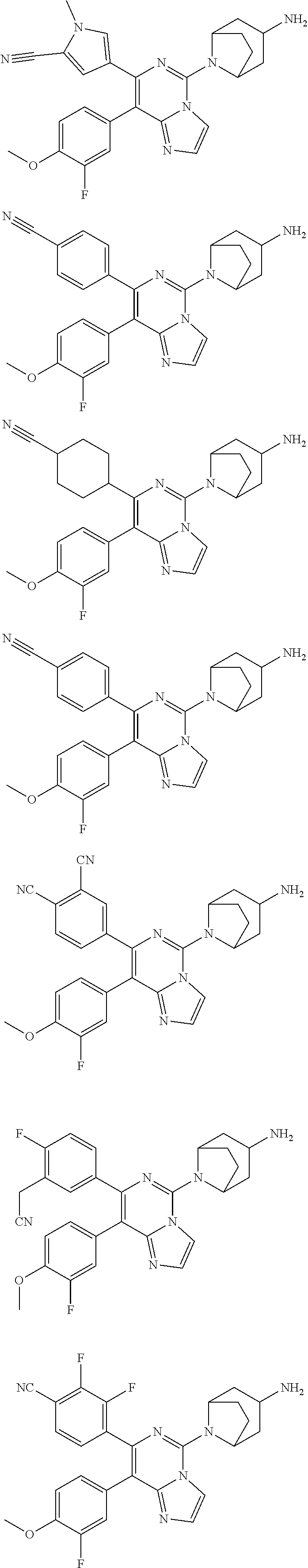

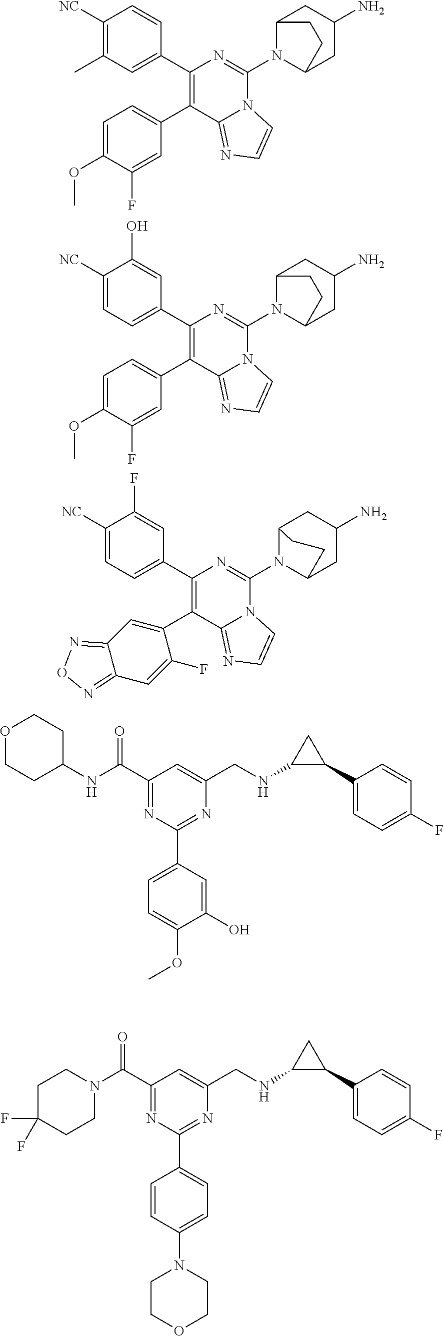

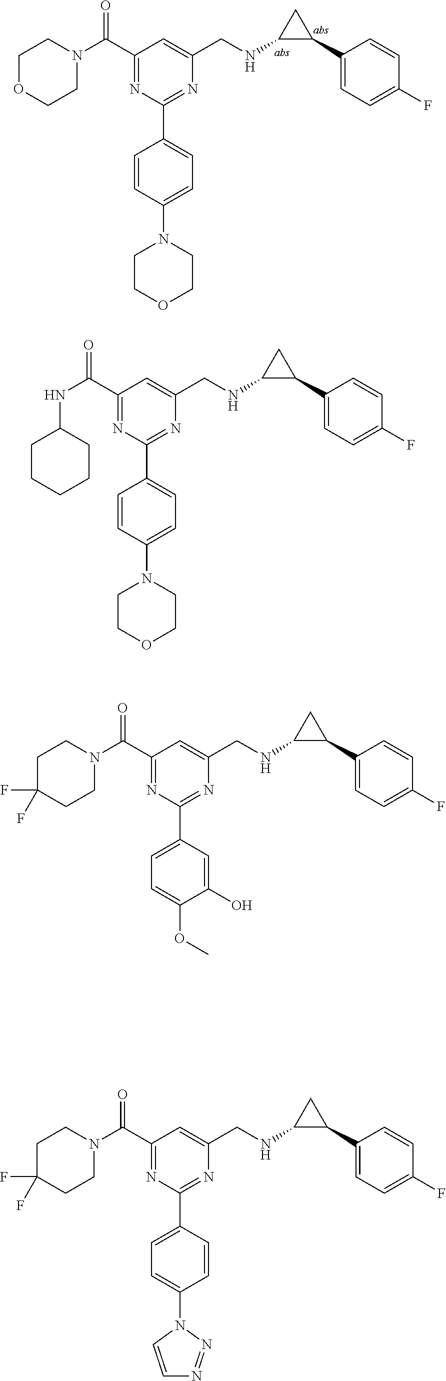 [SMILES: O=C(c1cc(CN[C@@H]2C[C@H]2c2ccc(F)cc2)nc(-c2ccc(N3CCOCC3)cc2)n1)N1CCOCC1 ; O=C(NC1CCCCC1)c1cc(CN[C@@H]2C[C@H]2c2ccc(F)cc2)nc(-c2ccc(N3CCOCC3)cc2)n1 ; COc1ccc(-c2nc(CN[C@@H]3C[C@H]3c3ccc(F)cc3)cc(C(=O)N3CCC(F)(F)CC3)n2)cc1O ; O=C(c1cc(CN[C@@H]2C[C@H]2c2ccc(F)cc2)nc(-c2ccc(-n3ccnn3)cc2)n1)N1CCC(F)(F)CC1]

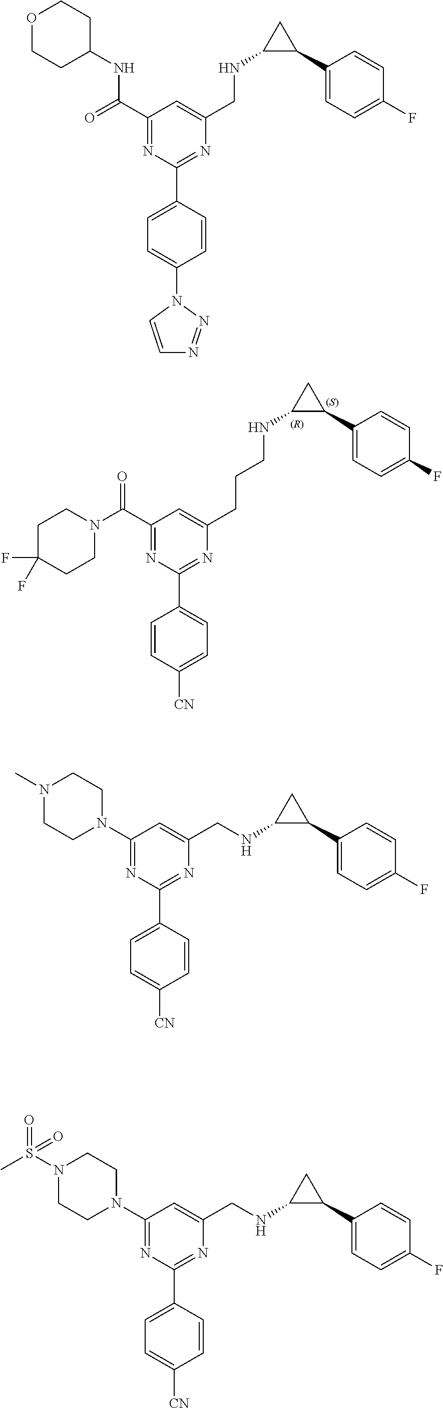

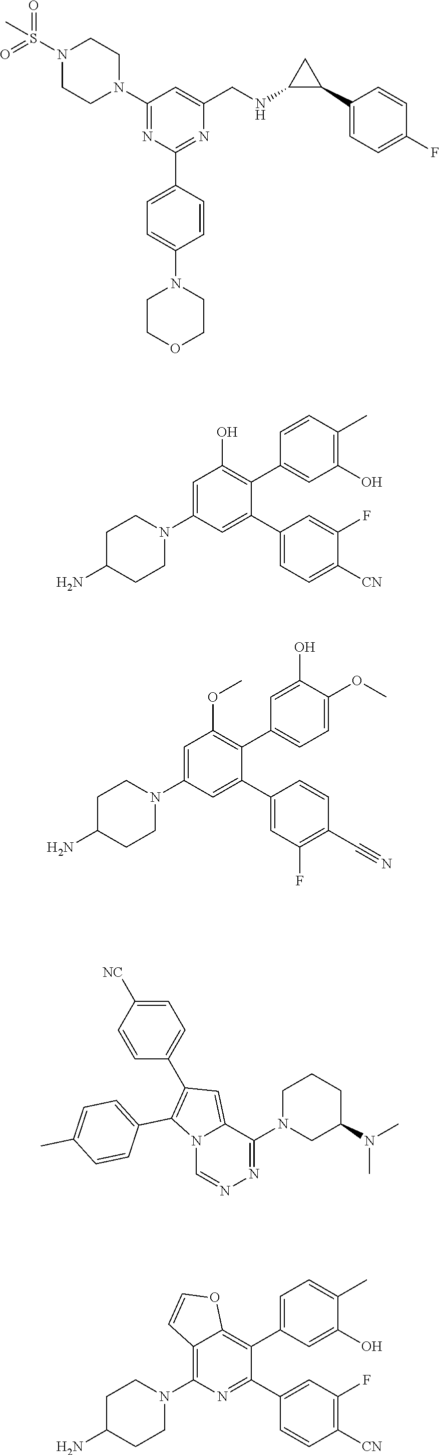

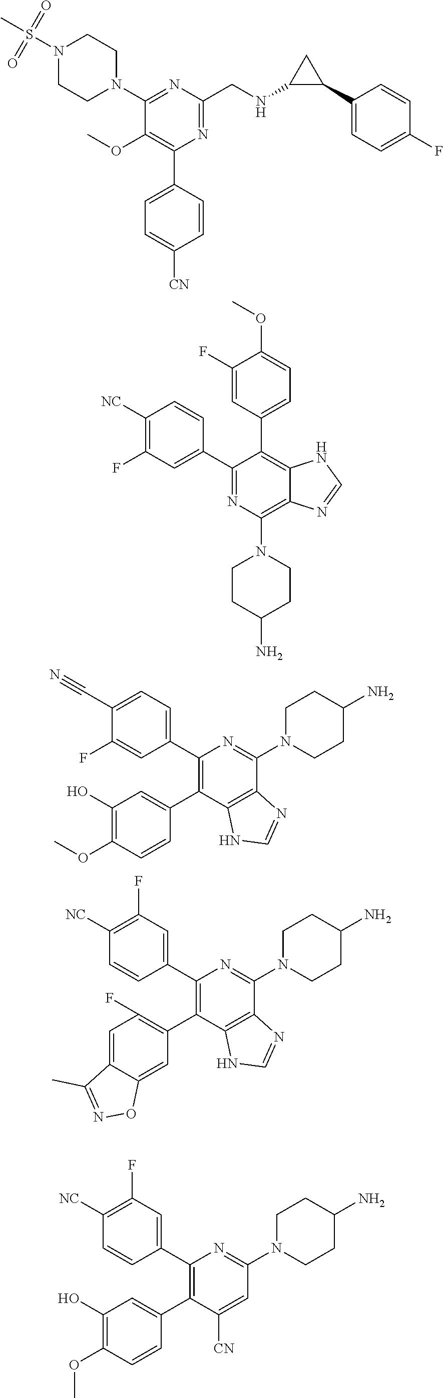

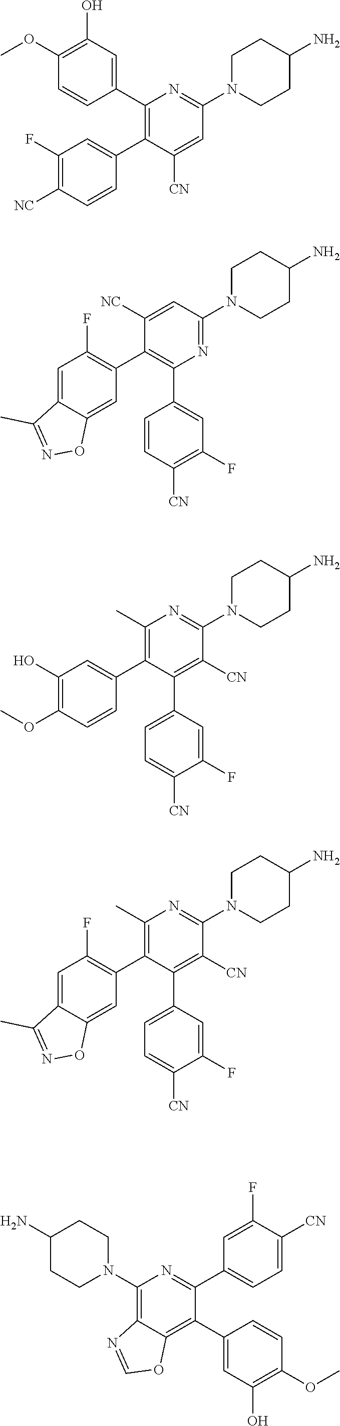 [SMILES: COc1ccc(-c2nc(N3CCC(N)CC3)cc(C#N)c2-c2ccc(C#N)c(F)c2)cc1O ; Cc1noc2cc(-c3c(C#N)cc(N4CCC(N)CC4)nc3-c3ccc(C#N)c(F)c3)c(F)cc12 ; COc1ccc(-c2c(C)nc(N3CCC(N)CC3)c(C#N)c2-c2ccc(C#N)c(F)c2)cc1O ; Cc1nc(N2CCC(N)CC2)c(C#N)c(-c2ccc(C#N)c(F)c2)c1-c1cc2onc(C)c2cc1F ; COc1ccc(-c2c(-c3ccc(C#N)c(F)c3)nc(N3CCC(N)CC3)c3ncoc23)cc1O]

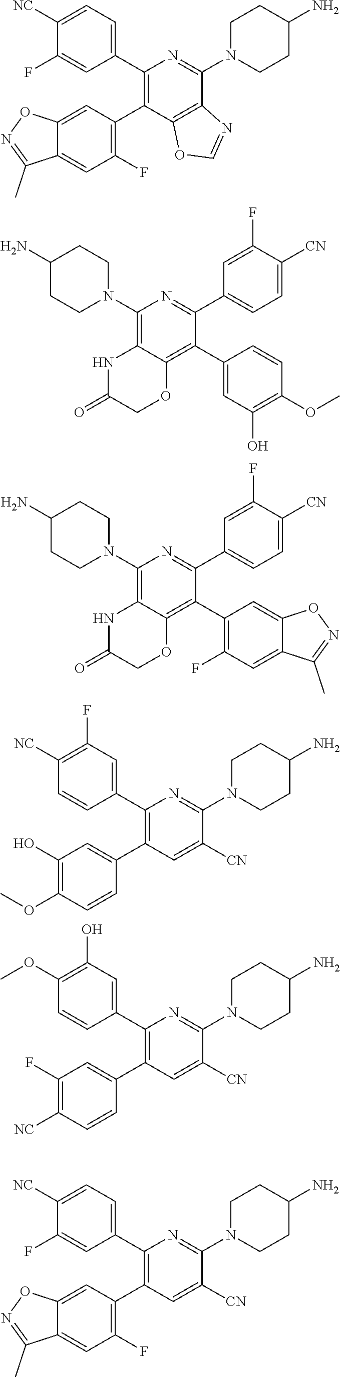

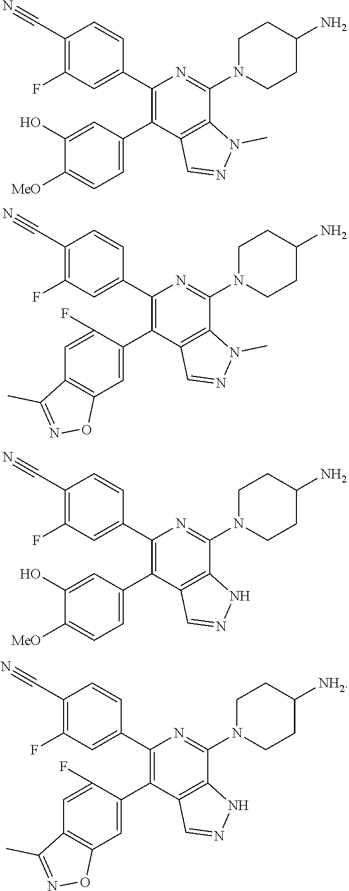

The present disclosure provides a pharmaceutical composition, the pharmaceutical composition comprising therapeutically effective amount of the above compounds or the tautomer, the mesomer, the racemate, the enantiomer, the diastereoisomer, or the mixture thereof, or the deuterated isotope derivative, the pharmaceutically acceptable hydrate, the solvate, the salt or the cocrystal thereof, and a pharmaceutically acceptable carrier, diluent, adjuvant, vehicle or excipient; the composition may further comprise one or more other therapeutic agents.

The present disclosure provides a use of the above compound or the tautomer, the mesomer, the racemate, the enantiomer, the diastereoisomer, or the mixture thereof, or the deuterated isotope derivative, the pharmaceutically acceptable hydrate, the solvate, the salt or the cocrystal thereof, or the above composition in the preparation of a medicament related to LSD1 inhibitor.

In an aspect of the present disclosure, the medicament related to LSD1 inhibitor is a medicament used for tumors; preferably, the medicament related to LSD1 inhibitor is a medicament used for lung cancer; and further preferably, the medicament related to LSD1 inhibitor is a medicament for small cell lung cancer.

In an aspect of the present disclosure wherein
ring A is selected from

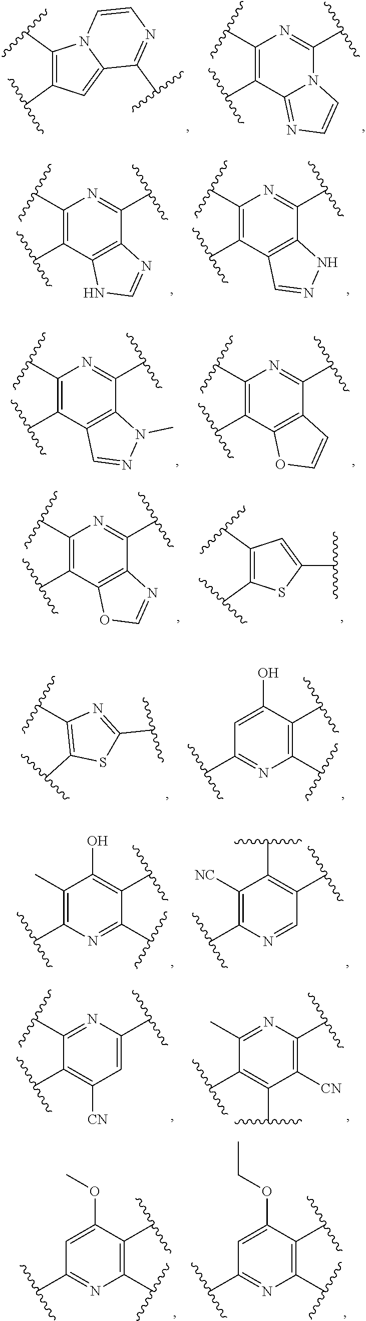

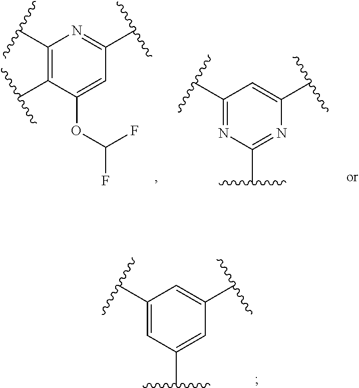 [SMILES: *c1cc(OC(F)F)c(*)c(*)n1 ; *c1cc(*)nc(*)n1 ; *c1cc(*)cc(*)c1]

ring B is selected from or

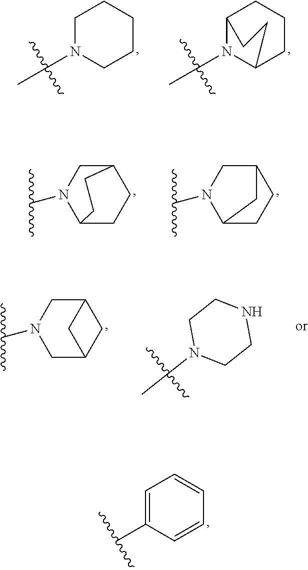

wherein the hydrogen of the group

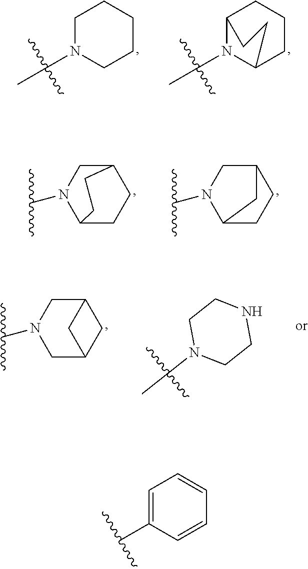

or is optionally substituted by 0 to 4 of halogen, NH₂, —NHCH₃ or methyl;
preferably, ring B is selected from

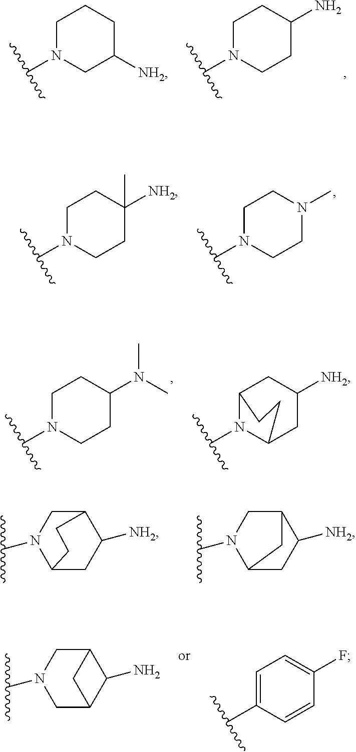

W is selected from a bond, —CH₂—,

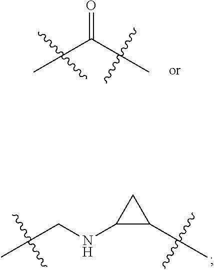

and
R₁ and/or R₂ is selected from:

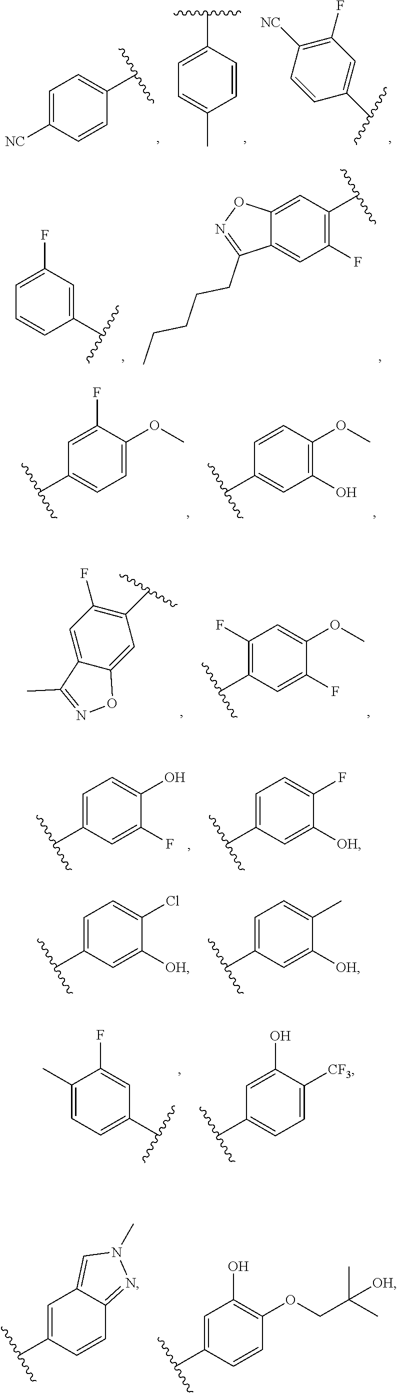

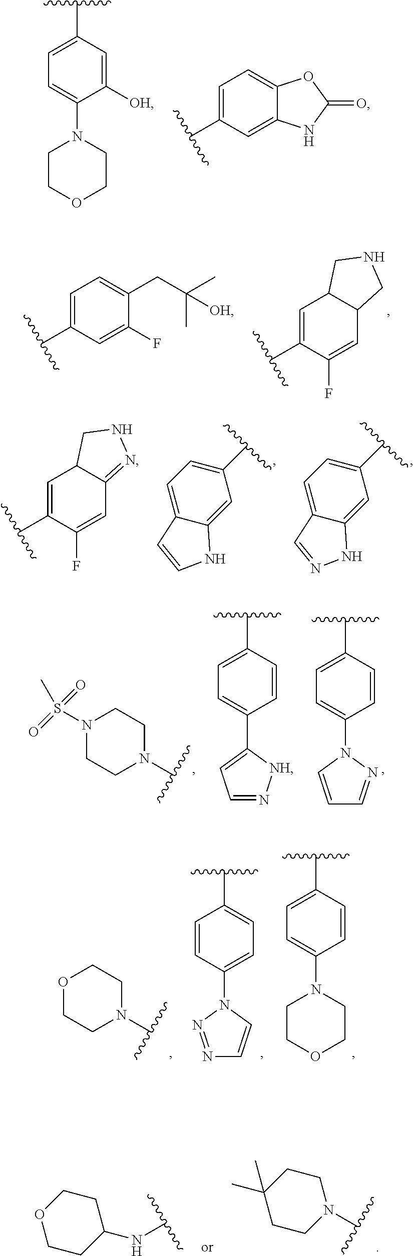

### DETAILED DESCRIPTION OF THE EMBODIMENTS

Unless otherwise defined, all technical terms and scientific terms used in the present disclosure have the same meaning as commonly understood by the person skilled in the art. Reference to the technical intention used herein refers to techniques generally known in the art, including those modifications of techniques or substitutions of equivalent techniques that are obvious to those skilled in the art. Although it is believed that the following terms are well understood by those skilled in the art, the following definitions are still provided to better explain the present invention.

### I. Definition

The terms “including”, “comprising” or “containing” and other variations herein are inclusive or open-ended, but do not exclude other unenumerated elements or method steps. Those skilled in the art should understand that the above terms such as “including” encompass the meaning of “consisting of”.

The term “one or more” or similar expressions “at least one” may denote for example, 1, 2, 3, 4, 5, 6, 7, 8, 9, 10 or more.

The term “aryl” refers to an all-carbon monocyclic or fused polycyclic aromatic group with a conjugated 7L electronic system. As used herein, the term “C₆₋₁₀ aryl” refers to an aromatic group containing 6 to 10 carbon atoms, such as phenyl or naphthoyl. The aryl may optionally substituted by one or more suitable substituents, such as cyano (CN) or halogen (F, Cl or Br).

The term “heteroaryl” refers to a monocyclic, bicyclic or tricyclic aromatic ring system, which comprises at least one heteroatom that may be same or different (the heteroatom is, for example, oxygen, nitrogen or sulfur), and additionally in each case, may be benzene fused. As used herein, the term “5-10-membered heteroaryl” refers to a monocyclic, bicyclic or tricyclic aromatic ring system having 5 to 10 atoms as ring atoms, which comprises at least one heteroatom that may be same or different (the heteroatom is, for example, oxygen, nitrogen or sulfur). The heteroaryl may optionally be substituted by one or more suitable substituents, such as cyano (CN) or halogen (F, Cl or Br).

The term “cycloalkyl” refers to a saturated monocyclic or polycyclic (e.g., bicyclic) hydrocarbon ring (e.g., monocyclic ring, such as cyclopropyl, cyclobutyl, cyclopentyl, cyclohexyl; or bicyclic ring, including a spiro ring, and a fused or bridging system (e.g., bicyclo[2.2.1]heptyl, etc.)). As used herein, the term “C₃₋₁₀ cycloalkyl” refers to a saturated monocyclic or polycyclic (e.g., bicyclic) hydrocarbon ring (e.g., cyclopropyl, cyclobutyl, cyclopentyl, or cyclohexyl) having 3 to 10 carbon atoms as ring atoms. The cycloalkyl may optionally be substituted by one or more suitable substituents.

The term “heterocycloalkyl” refers to a saturated monocyclic or polycyclic (e.g., bicyclic) group having 2, 3, 4, 5, 6, 7, 8, 9 or 10 carbon atoms and one or more heteroatoms in the ring; and the heterocycloalkyl may be linked to the rest of the molecule through any one of the carbon atoms or the heteroatoms. As used herein, the term “C₂₋₁₀ heterocycloalkyl” refers to a saturated monocyclic or polycyclic (e.g., bicyclic) group having 2 to 10 carbon atoms as ring atoms, which comprises at least one heteroatom that may be same or different (the heteroatom is, for example, oxygen, nitrogen or sulfur). The heterocycloalkyl may optionally be substituted by one or more suitable substituents.

The term “cycloalkenyl” refers to a non-aromatic monocyclic or polycyclic (e.g., bicyclic) hydrocarbon ring containing at least one carbon-carbon double bond (i.e., C═C). As used herein, the term C₃₋₁₀ cycloalkenyl refers to unsaturated non-aromatic alicyclic hydrocarbon having 3 to 10 carbon atoms as ring atoms, and examples include, but are not limited to, cyclopropenyl, cyclobutenyl, cyclopentenyl, cyclopentadienyl, cyclohexenyl, cyclohexadienyl, etc. The cycloalkenyl may optionally be substituted by one or more suitable substituents.

The term “heterocycloalkenyl” refers to a class of cycloalkenyl defined above, and at least one carbon atom of its ring is substituted by a heteroatom, such as nitrogen, oxygen or sulfur. Examples of C₂₋₁₀ heterocycloalkenyl include, but are not limited to, tetrahydropyridine, dihydropyran, dihydrofuran, pyrroline, etc., which may be monocyclic or polycyclic (e.g., bicyclic) groups. The heterocycloalkenyl may optionally be substituted by one or more suitable substituents.

The term “halogenated” or “halogen” group is defined to include F, Cl, Br or I.

The term “amino” refers to —NH₂.

The term “hydroxy” refers to —OH.

The term “alkyl” is defined as a straight-chain or branched-chain saturated aliphatic hydrocarbon group. As used herein, the term “C₁₋₆ alkyl” refers to a straight-chain or branched-chain saturated aliphatic hydrocarbon group having 1, 2, 3, 4, 5 or 6 carbon atoms, such as methyl, ethyl, n-propyl, isopropyl, n-butyl, sec-butyl, tert-butyl, n-pentyl, n-hexyl, and the like.

The term “halogen substituted alkyl”, when used herein alone or in combination with other groups, refers to the alkyl defined above, of which one or more hydrogen atoms are substituted by halogen. Those skilled in the art should understand that when there are more than one halogen substituents, the halogen may be the same or different, and may be located on the same or different carbon atoms. As used herein, the term “halogen substituted C₁₋₆ alkyl” refers to one or more hydrogen atoms of C₁₋₆ alkyl being substituted by halogen, such as trifluoromethyl.

The term “hydroxy substituted alkyl” refers to the alkyl as defined above, of which one or more hydrogen atoms are substituted by hydroxy. As used herein, the term “hydroxy substituted C₁₋₆ alkyl” refers to one or more hydrogen atoms of C₁₋₆ alkyl being substituted by hydroxy, such as

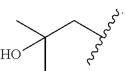 [SMILES: *CC(C)(C)O]

The term “amino(—NH₂) substituted alkyl” refers to the alkyl defined above, of which one or more hydrogen atoms are substituted by amino(—NH₂). As used herein, the term “amino(—NH₂) substituted C₁₋₆ alkyl” refers to one or more hydrogen atoms of C₁₋₆ alkyl being substituted by amino(—NH₂), such as NH₂

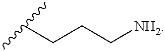 [SMILES: *CCCN]

The term “alkoxy substituted alkyl” refers to the alkyl as defined above, of which one or more hydrogen atoms are substituted by alkoxy. As used herein, the term “C₁₋₆ alkoxy substituted C₁₋₆ alkyl” refers to one or more hydrogen atoms of the C₁₋₆ alkyl being replaced by C₁₋₆ alkoxy; and the term “C₁₋₃ alkoxy substituted C¹⁻³ alkyl” refers to one or more hydrogen atoms of the C₁₋₃ alkyl being substituted by C₁₋₃ alkoxy, such as.

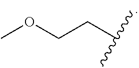 [SMILES: *CCOC]

The term “alkoxy” refers to the connection of oxygen atoms to “alkyl” as defined above, i.e., the “alkoxy” group may be defined as —OR, wherein R is the alkyl as defined above. As used herein, examples of the term “C₁₋₆ alkoxy” include, but are not limited to, methoxy, ethoxy, n-propoxy, isopropoxy, n-butoxy, sec-butoxy, tert-butoxy, n-pentyloxy, n-hexyloxy, etc.

The term “halogen substituted alkoxy” refers to the alkoxy as defined above, in which one or more hydrogen atoms are substituted by halogen. Those skilled in the art should understand that when there are more than one halogen substituents, the halogen may be the same or different, and may be located on the same or different carbon atoms. As used herein, the term “halogen substituted C₁₋₆ alkoxy” refers to one or more hydrogen atoms of C₁₋₆ alkoxy being substituted by halogen, such as difluoromethoxy, and trifluoromethoxy.

The term “aryl substituted alkoxy” refers to the alkoxy as defined above, of which one or more hydrogen atoms are substituted by aryl. As used herein, the term “C₆₋₁₀ aryl substituted C₁₋₆ alkoxy” refers to one or more hydrogen atoms of C₁₋₆ alkoxy being substituted by C₆₋₁₀ aryl, such as benzyloxy

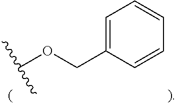

The term “hydroxy substituted alkoxy” refers to the alkoxy as defined above, of which one or more hydrogen atoms are substituted by hydroxy. As used herein, the term “hydroxy substituted C₁₋₆ alkoxy” refers to one or more hydrogen atoms of C₁₋₆ alkoxy being substituted by hydroxy, such as

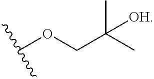 [SMILES: *OCC(C)(C)O]

The term “—O cycloalkyl” refers to the connection of oxygen atoms to the “cycloalkyl” as defined above, i.e., the “—O cycloalkyl” group may be defined as —OR, where R is the cycloalkyl as defined above. As used herein, examples of the term “—OC₃₋₁₀ cycloalkyl” may be, for example, cyclopentenyl-oxy-

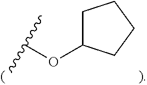 [SMILES: *OC1CCCC1]

The term “—O aryl” refers to the connection of oxygen atoms to the “aryl” as defined above, i.e., the “—O aryl” group may be defined as —OR, where R is the aryl as defined above. As used herein, examples of the term “—OC₆₋₁₀ aryl” may be, for example, phenoxy

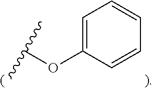 [SMILES: *Oc1ccccc1]

The term “—O heteroaryl” refers to the connection of oxygen atoms to the “heteroaryl” as defined above, i.e., the “—O heteroaryl” group may be defined as —OR, where R is the heteroaryl as defined above.

The term “alkenyl” refers to a straight-chain or branched-chain aliphatic hydrocarbon group containing at least one carbon-carbon double bond. The double bond may exist as an E or Z isomer. The double bond may be located anywhere possible in the hydrocarbon chain. As used herein, the term “C₂₋₆ alkenyl” refers to alkenyl containing 2 to 6 carbon atoms, such as vinyl, propenyl, butenyl, butadienyl, pentenyl, pentadienyl, hexenyl, hexadienyl, etc. The alkenyl may optionally be substituted by one or more suitable substituents.

The term “alkynyl” refers to a straight-chain or branched-chain aliphatic hydrocarbon group containing at least one C≡C triple bond. The triple bond may be located anywhere possible in the hydrocarbon chain. As used herein, the term “C₂₋₆ alkynyl” refers to alkynyl containing 2 to 6 carbon atoms, such as ethynyl, propynyl, butynyl, pentynal, hexynyl, etc. The alkynyl may optionally be substituted by one or more suitable substituents.

The term “hydroxy substituted alkynyl” refers to the alkynyl as defined above, of which one or more hydrogen atoms are substituted by hydroxy. As used herein, the term “hydroxy substituted C₂₋₆ alkynyl” refers to one or more hydrogen atoms of C₂₋₆ alkynyl being substituted by hydroxy, such as

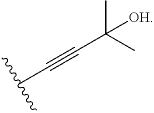 [SMILES: *C#CC(C)(C)O]

The term “aminoalkyl”, when used herein alone or in combination with other groups, refers to the alkyl defined above, of which one or more hydrogen atoms are substituted by amino.

The term “aminocycloalkyl”, when used herein alone or in combination with other groups, refers to the cycloalkyl defined above, of which one or more hydrogen atoms are substituted by amino.

The term “aminoheterocycloalkyl”, when used herein alone or in combination with other groups, refers to the heterocycloalkyl defined above, of which one or more hydrogen atoms are substituted by amino.

The term “substituted” means that one or more (e.g., one, two, three, or four) hydrogens of a specified atom are optionally substituted by the indicated groups, provided that the normal atomic valence of the specified atom under the present circumstances is not exceeded and the substitution forms a stable compound. The combination of substituents and/or variants is only allowed if such combination form a stable compound.

The term “optionally substituted” refers to the optional substitution by a particular group, atomic group, or partial substitution.

When a group is described as “optionally substituted by one or more substituents”, this group may be (1) unsubstituted or (2) substituted. If carbon on a group is described as being optionally substituted by one or more substituents, one or more hydrogen on the carbon (to the extent of any hydrogen present) may be individually and/or together substituted by substituted or unsubstituted by the independently selected substituents. If carbon on a group is described as being optionally substituted by one or more substituents, one or more hydrogen on the carbon (to the extent of any hydrogen present) may be each substituted or unsubstituted by the independently selected substituents.

When a bond of a substituent appears as a bond connecting two atoms and passing through the ring, such a substituent may bond to any ring-forming atom in the ring which can be substituted.

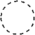

indicates the presence or absence of the possible double bond at any position of the ring, which means that it includes the presence of a plurality of cases, such as a saturated ring, an unsaturated non-aromatic rings with double bonds, and an aromatic ring.

The compounds of the present invention may further comprise one or more (e.g., one, two, three, or four) isotope displacements.

The term “stereoisomer” represents an isomer formed due to at least one asymmetric center. Compounds with one or more (e.g., one, two, three, or four) asymmetric centers may produce racemate, racemic mixture, single enantiomers, diastereoisomer mixtures, and individual diastereoisomers. Specific individual molecules may also exist as geometric isomers (cis/trans). Similarly, the compounds of the present invention may exist in two or more mixtures of structurally different forms (commonly referred to as tautomers) in rapid equilibrium. Representative examples of tautomers include ketone-enol tautomers, phenol-ketone tautomers, nitroso-oxime tautomers, imine-enamine tautomers, etc. The scope of the present application covers all such isomers and their mixtures in any proportion (e.g., 60%, 65%, 70%, 75%, 80%, 85%, 90%, 95%, 96%, 97%, 98%, 99%).

Pharmaceutically acceptable salts of the compounds of the present disclosure may include acid addition salts and alkali salts of the compounds. Suitable acid addition salts are formed from acids that form non-toxic salts. Examples include acetate, aspartate, benzoate, benzenesulfonate, bicarbonate/carbonate, bisulfate/sulfate, borate, camphorsulfonate, citrate, ethanedisulfonate, ethanesulfonate, formate, fumarate, glucoheptonate, gluconate, glucuronate, hexafluorophosphate, hibenzate, hydrochloride/chloride, hydrobromide/bromide, hydroiodate/iodide, isethionate, lactate, malate, maleate, malonate, mesylate, methyl sulfate, naphthoate, 1,5-naphthalenedisulfonate, 2-naphthalenesulfonate, nicotinate, nitrate, orotate, oxalate, palmitate, pamoate, phosphate/biphosphate/dihydrogen phosphate, saccharate, stearate, succinate, tartrate, tosylate, and trifluoroacetate. Suitable alkali salts are formed from alkali that form non-toxic salts. Examples include aluminum, arginine, benzathine, calcium, choline, diethylamine, diethanolamine, glycine, lysine, magnesium, meglumine, ethanolamine, potassium, sodium, trometamol, and zinc salts. Semi-salts of acids and bases, such as hemisulphate and semi-calcium salts, may also be formed. For a review of suitable salts, see “Handbook of Pharmaceutical Salts: Properties, Selection, and Use” by Stahl and Wermuth (Wiley-VCH, Weinheim, Germany, 2002).

### II. Examples

The following details the implementation process of the present invention and the beneficial effects produced by specific embodiments, and in order to help the readers better understand the essence and characteristics of the present invention, and is not used as a limitation of the scope of implementation in the present case.

The structures of compounds are determined by nuclear magnetic resonance (NMR) or/and mass spectrometry (MS). NMR shift (6) is given in unit of 10-6 ppm. NMR is determined using an AVANCE NEO 400 MHz Bruker instrument, and solvents for determination are deuterated dimethyl sulfoxide (DMSO-d₆), deuterated chloroform (CDCl₃), and deuterated methanol (CD₃OD), and an internal standard substance is tetramethylsilane (TMS). MS is obtained from an ISQ-EC Thermo Fisher LC-MS instrument. An instrument for preparative HPLC is a GX-281 Gilson chromatograph. A separation method includes: (1) 5 μL of Sun Fire® Prep C18 OBDTM, 30×150 mm Column, 0.04% HCl aqueous solution/acetonitrile; (2) 5 μL of Sun Fire® Prep C18 OBDTM, 30×150 mm Column, 0.02% TFA aqueous solution/acetonitrile; (3) 5 μL of Sun Fire® Prep C18 OBDTM, 30x150 mm Column, 0.06% formic acid aqueous solution/acetonitrile; (4) 5 μL of Xbridge® Prep C18 OBDTM, 30×150 mm Column, 10 mM NH₄HCO₃ aqueous solution/acetonitrile; and (5) 5 μL of Xbridge® Prep C18 OBDTM, 30×150 mm Column, 0.6% NH₃·H₂O aqueous solution/acetonitrile.

The solvents used in the present invention are commercially available.

The solutions in the examples refer to aqueous solutions, unless otherwise specified.

The reaction temperature in the examples is room temperature ranging from 20° C. to 30° C., unless otherwise specified.
HATU refers to 0-(7-azabenzotriazole-1-yl)—N,N,N′,N′-tetramethylurea hexafluorophosphate;
DIPEA refers to diisopropylethylamine;
DMSO refers to dimethyl sulfoxide;
Pd₂(dba)₃ refers to tris(dibenzylideneacetone)dipalladium;
DCE refers to dichloroethane;
DCM refers to dichloromethane;
DMF refers to N,N-dimethylformamide;
NMP refers to N-methylpyrrolidone;
TFA refers to trifluoroacetic acid;
NBS refers to N-bromosuccinimide;
TfOH refers to trifluoromethanesulfonic acid; and
DPPA refers to diphenylphosphoryl azide.

### Example 1

### Preparation of (6-(((1R,2S)-2-(4-fluorophenyl)cyclopropyl)amino)methyl)-2-phenylpyrimidin-4-yl)(4-(methyls ulfonyl)piperazin-1-yl)methanone

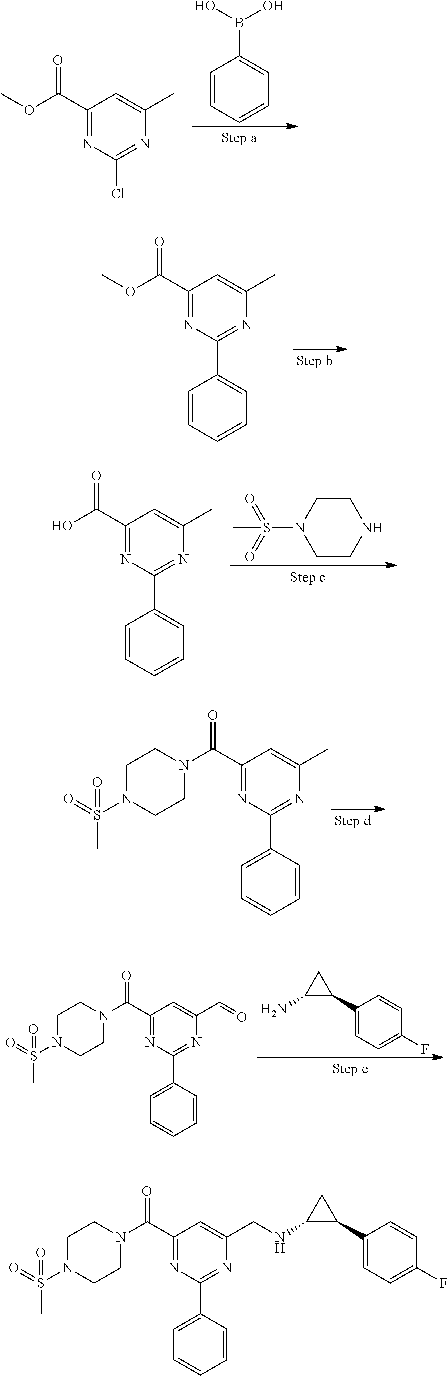

### Step a): Preparation of methyl 6-methyl-2-phenylpyrimidine-4-carboxylate

Methyl 2-chloro-6-methylpyrimidine-4-carboxylate (1.0 g, 5.3 mmol), Pd₂(dba)₃ (73 mg, 0.08 mmol), P(t-Bu)₃ (1.95 g, 9.6 mmol), KF (934 mg, 9.3 mmol) and phenylboronic acid (1.044 g, 8.0 mmol) are quickly added to a flask, and then tetrahydrofuran (10 mL) was added by using a syringe, the flask is evacuated and backfilled with nitrogen three times, and the mixture is stirred at 90° C. for 8 h under nitrogen atmosphere. When LC-MS shows that the reaction is completed, a crude product is obtained by concentration, and the residue is purified by silica gel chromatography (eluent: petroleum ether/ethyl acetate=10/3) to obtain methyl 6-methyl-2-phenylpyrimidine-4-carboxylate with a yield of 98.1%.

¹H NMR (400 MHz, DMSO-d₆) δ ppm 8.40 (dd, J=4.8, 2.8 Hz, 2H), 7.83 (m, 1H), 7.60-7.39 (m, 3H), 3.94 (m, 3H), 2.62 (m, 3H).

ESI-MS m/z:229.1 [M+H]⁺.

### Step b): Preparation of 6-methyl-2-phenylpyrimidine-4-carboxylic acid

Methyl 6-methyl-2-phenylpyrimidine-4-carboxylate (1.2 g, 5.2 mmol) is dissolved in a tetrahydrofuran (10 mL) solution, added with a LiOH—H₂O (0.88 g, 21.1 mmol) aqueous solution at 0° C., stirred for 0.5 h, warmed to room temperature and stirred for 4 h. When LS-MS shows that the reaction is completed, impurities are extracted with petroleum ether/ethyl acetate=4:1 (20 mL), the aqueous phase is adjusted to pH of about 4-5 by HCl(1N) and extracted with ethyl acetate (20 mL×3), and the organic layer is washed with saturated saline (20 mL), dried with anhydrous sodium sulfate, filtered, and concentrated under a reduced pressure to obtain 6-methyl-2-phenylpyrimidine-4-carboxylic acid with a yield of 80.7%.

¹H NMR (400 MHz, DMSO-d₆) δ ppm 13.79 (s, 1H), 8.47 (dt, J=7.4, 3.6 Hz, 2H), 7.83 (s, 1H), 7.66-7.45 (m, 3H), 2.65 (s, 3H).

ESI-MS m/z: 214.2 [M+H]⁺.

### Step c): Preparation of (6-methyl-2-phenylpyrimidin-4-yl) (4-(methylsulfonyl)piperazin-1-yl)methanone

6-Methyl-2-phenylpyrimidine-4-carboxylic acid (1.0 g, 4.8 mmol) and 1-(methylsulfonyl)piperazine (782 mg, 4.8 mmol) are added to DMF (20 mL), added with HATU (1.81 g, 4.8 mmol) and DIPEA (1.54 g, 11.9 mmol) at 0° C., the reaction was warmed to room temperature and stirred for 1 h; when LC-MS shows that the reaction is completed, the reaction solution is quenched with 40 mL of water, and extracted with ethyl acetate (40 mL×2); organic phases are combined, washed with saturated saline (30 mL×2), dried with anhydrous sodium sulfate, filtered, and concentrated to obtain a crude product; and the residue is purified by silica gel chromatography (eluent: dichloromethane/ethyl acetate=10/7) to obtain (6-methyl-2-phenylpyrimidin-4-yl)(4-(methylsulfonyl)piperazin-1-yl) methanone with a yield of 87.5%.

¹H NMR (400 MHz, DMSO-d₆) δ ppm 8.40 (dt, J=7.8, 3.8 Hz, 2H), 7.65-7.47 (m, 4H), 3.86-3.73 (m, 2H), 3.66-3.53 (m, 2H), 3.32-3.25 (m, 2H), 3.19 (d, J=26.8 Hz, 2H), 2.95 (s, 3H), 2.62 (s, 3H).

ESI-MS m/z: 361.4 [M+H]⁺.

### Step d): Preparation of 6-(4-(methylsulfonyl)piperazine-1-carbonyl)-2-phenylpyrimidine-4-formaldehyde

(6-Methyl-2-phenylpyrimidin-4-yl)(4-(methylsulfonyl)piperazin-1-yl) methanone (500 mg, 1.4 mmol) and SeO₂ (740 mg, 6.66 mmol) are added to a reaction flask containing 1,4-dioxane (15 mL), the reaction temperature was warmed to 110° C., and stirred for 8 h. When LC-MS shows that the reaction is completed, the reaction solution is quenched with a saturated sodium bicarbonate aqueous solution (40 mL), and extracted with ethyl acetate (40 mL×2); organic phases are combined, washed with saturated saline (40 mL×2), dried with anhydrous sodium sulfate, filtered, and concentrated to obtain a crude product, i.e., 6-(4-(methylsulfonyl)piperazine-1-carbonyl)-2-phenylpyrimidine-4-formaldehyde with a yield of 66.4%.

¹H NMR (400 MHz, DMSO-d₆) δ ppm 10.07 (s, 1H), 8.56-8.31 (m, 2H), 7.93 (s, 1H), 7.77-7.47 (m, 3H), 3.89-3.71 (m, 2H), 3.70-3.53 (m, 2H), 3.35-3.15 (m, 4H), 2.95 (s, 3H).

ESI-MS m/z:393 [M+H₂O]⁺.

### Step e): Preparation of (6-((((1R,2S)-2-(4-fluorophenyl)cyclopropyl)amino)methyl)-2-phenylpyrimidin-4-yl)(4-(methylsulfonyl)p iperazin-1-yl)methanone

6-(4-(methylsulfonyl)piperazine-1-carbonyl)-2-phenylpyrimidine-4-formaldehyde (100 mg, 0.27 mmol) is added to DCE (2 mL), and stirred at 25° C. for 3 h. The mixture is sequentially added with MeOH (0.8 mL), AcOH (0.1 mL) and (1R,2S)-2-(4-fluorophenyl)cyclopropyl-1-amine (60 mg, 0.27 mmol) and stirred at room temperature for 2 h. When LC-MS shows that the reaction is completed, the reaction solution is added with NaBH₃CN (67 mg, 1.07 mmol); and when LC-MS shows that the reaction is completed, a crude product is obtained by concentration and delivered to Prep-HPLC for preparation (separation method 4) to obtain (6-((((1R,2S)-2-(4-fluorophenyl)cyclopropyl)amino)methyl)-2-phenylpyrimidin-4-yl)(4-(methyls ulfonyl)piperazin-1-yl)methanone with a yield of 18.5%.

¹H NMR (400 MHz, Methanol-d₄) δ ppm 8.44 (d, J=7.6 Hz, 2H), 7.58-7.43 (m, 4H), 7.02 (dd, J=8.2, 5.6 Hz, 2H), 6.90 (t, J=8.6 Hz, 2H), 4.12 (s, 2H), 4.07-3.84 (m, 2H), 3.74-3.63 (m, 2H), 3.59-3.38 (m, 2H), 3.40 (d, J=5.0 Hz, 2H), 2.90 (s, 3H), 2.42 (dt, J=7.2, 3.8 Hz, 1H), 2.32-1.91 (m, 1H), 1.22-1.09 (m, 1H), 1.00 (dd, J=12.4, 6.2 Hz, 1H).

ESI-MS m/z:510.2 [M+H]⁺.

### Example 2

### Preparation of (2-(4-(1H-pyrazol-1-yl)phenyl)-6-((((1R,2S)-2-(4-fluorophenyl)cyclopropyl)amino)methyl)pyri midin-4-yl)(4-(methylsulfonyl)piperazin-1-yl)methanone

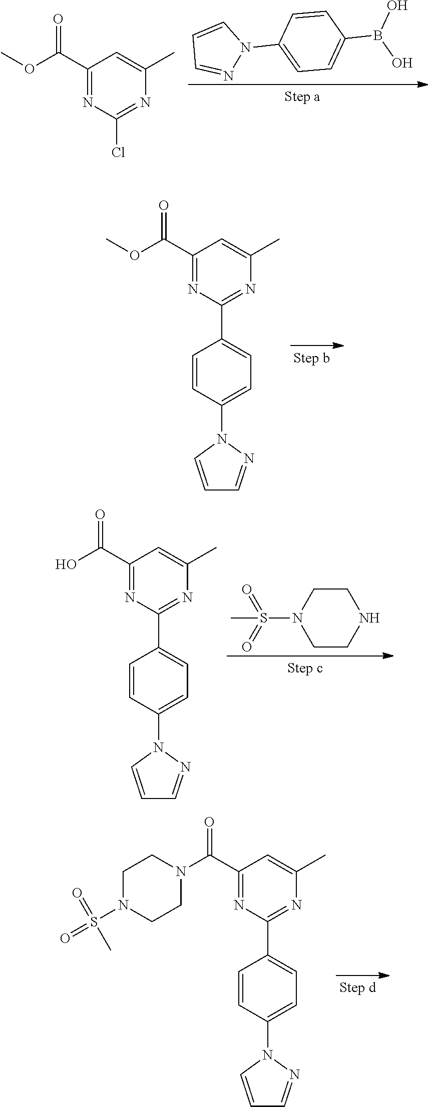

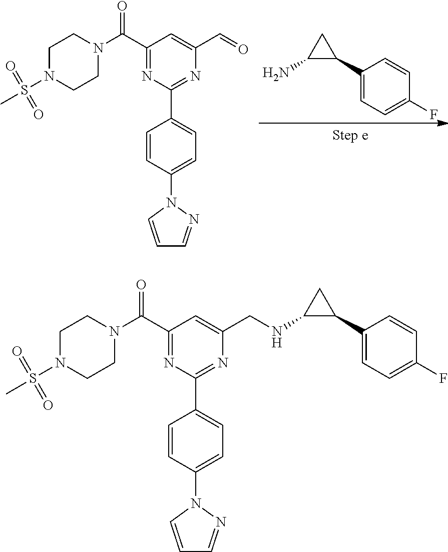

### Step a): Preparation of methyl 2-(4-(1H-pyrazol-1-yl)phenyl)-6-methylpyrimidine-4-carboxylate

Methyl 2-chloro-6-methylpyrimidine-4-carboxylate (1 g, 5.3 mmol), Pd₂(dba)₃ (73 mg, 0.08 mmol), P(t-Bu)₃ (1.95 g, 9.6 mmol), KF (934 mg, 9.3 mmol) and (4-(1H-pyrazol-1-yl)phenyl)boronic acid (1.5 g, 8.0 mmol) are quickly added to a flask, and then tetrahydrofuran (10 mL) was added by using a syringe, the flask is evacuated and backfilled with nitrogen three times, and the mixture is stirred at 90° C. for 8 h under nitrogen atmosphere. When LC-MS shows that the reaction is completed, a crude product is obtained by concentration, and the residue is purified by silica gel chromatography (eluent: petroleum ether/ethyl acetate=10/7) to obtain methyl 2-(4-(1H-pyrazol-1-yl)phenyl)-6-methylpyrimidine-4-carboxylate with a yield of 84.9%.

¹H NMR (400 MHz, DMSO-d₆) δ ppm 8.85-8.36 (m, 3H), 8.24-7.67 (m, 4H), 6.60 (s, 1H), 3.96 (s, 3H), 2.65 (s, 3H).

ESI-MS m/z:295.2[M+H]⁺.

### Step b): Preparation of 2-(4-(1H-pyrazol-1-yl)phenyl)-6-methylpyrimidine-4-carboxylic acid

Methyl 2-(4-(1H-pyrazol-1-yl)phenyl)-6-methylpyrimidine-4-carboxylate (1.5 g, 5.1 mmol) is added to tetrahydrofuran (10 mL), and a LiOH—H₂O (299 mg, 7.14 mmol) aqueous solution is added to the mixed solution at 0° C., stirred for 0.5 h, then warmed to room temperature and continuously stirred for 4 h. When LS-MS shows that the reaction is completed, impurities are extracted with petroleum ether/ethyl acetate=4:1 (20 mL), an aqueous phase is adjusted to pH of about 4-5 by HCl (1N) and extracted with ethyl acetate (20 mL×3) to obtain a solution of the product, and the organic layer is washed with saturated saline (20 mL), dried with anhydrous sodium sulfate, filtered, and concentrated under a reduced pressure to obtain 2-(4-(1H-pyrazol-1-yl)phenyl)-6-methylpyrimidine-4-carboxylic acid with a yield of 89.5%.

¹H NMR (400 MHz, DMSO-d₆) δ ppm 13.82 (s, 1H), 8.60 (dd, J=27.4, 5.6 Hz, 3H), 8.04 (d, J=8.8 Hz, 2H), 7.82 (s, 2H), 6.61 (d, J=1.8 Hz, 1H), 2.66 (s, 3H).

ESI-MS m/z:281.1 [M+H]⁺.

### Step c): Preparation of (2-(4-(1h-pyrazol-1-yl)phenyl)-6-methylpyrimidin-4-yl) (4-(methylsulfonyl)piperazin-1-yl)methanone

2-(4-(1H-pyrazol-1-yl)phenyl)-6-methylpyrimidin-4-carboxylic acid (900 mg, 3.21 mmol) and 1-(methylsulfonyl)piperazine (633 mg, 3.85 mmol) are dissolved in DMF (20 mL), added with HATU (1.470 g, 3.85 mmol) and DIPEA (1.25 g, 9.64 mmol) at 0° C., the reaction was warmed to room temperature and stirred for 1 h; when LC-MS shows that the reaction is completed, the mixture is quenched with 40 mL of water, and extracted with ethyl acetate (40 mL×2); organic phases are combined, washed with saturated saline (30 mL×2), dried with anhydrous sodium sulfate, filtered, and concentrated to obtain a crude product; and the residue is purified by silica gel chromatography (eluent: dichloromethane/ethyl acetate=10/7) to obtain (2-(4-(1h-pyrazol-1-yl)phenyl)-6-methylpyrimidin-4-yl) (4-(methylsulfonyl)piperazin-1-yl) methanone with a yield of 86.3%.

¹H NMR (400 MHz, DMSO-d₆) δ ppm 8.63 (d, J=2.6 Hz, 1H), 8.50 (d, J=8.8 Hz, 2H), 8.01 (t, J=18.5 Hz, 2H), 7.82 (d, J=1.4 Hz, 1H), 7.50 (s, 1H), 6.61 (t, J=1.8 Hz, 1H), 3.86-3.76 (m, 2H), 3.67-3.54 (m, 2H), 3.32-3.25 (m, 2H), 3.25-3.17 (m, 2H), 2.96 (s, 3H), 2.63 (s, 3H).

ESI-MS m/z:427.3 [M+H]⁺.

### Step d): Preparation of 2-(4-(1h-pyrazol-1-yl)phenyl)-6-(4-(methylsulfonyl)piperazin-1-carbonyl)pyrimidine-4-formalde hyde

(2-(4-(1h-pyrazol-1-yl)phenyl)-6-methylpyrimidin-4-yl) (4-(methylsulfonyl)piperazin-1-yl)methanone (1.2 g, 2.8 mmol) is added to 1,4-dioxane (25 mL), added with SeO₂ (6.66 g, 60 mmol) at 25° C., the reaction temperature was warmed to 110° C., and stirred for 8 h; when LC-MS shows that the reaction is completed, the reaction solution is quenched with a saturated sodium bicarbonate aqueous solution (40 mL), and extracted with ethyl acetate (40 mL×2); organic phases are combined, washed with saturated saline (40 mL×2), dried with anhydrous sodium sulfate, filtered, and concentrated to obtain a crude product, i.e., 2-(4-(1H-pyrazol-1-yl)phenyl)-6-(4-(methylsulfonyl)piperazin-1-carbonyl)pyrimidine-4-formald ehyde with a yield of 65.8%.

¹H NMR (400 MHz, DMSO-d₆) δ ppm 10.09 (s, 1H), 8.69-8.43 (m, 3H), 8.17-7.99 (m, 2H), 7.94-7.76 (m, 1H), 6.74-6.47 (m, 1H), 3.94-3.70 (m, 2H), 3.61 (dd, J=15.8, 10.9 Hz, 2H), 3.40-3.24 (m, 4H), 3.24-3.12 (m, 2H), 2.96 (s, 3H).

ESI-MS m/z:459.2 [M+H₂O]⁺.

### Step e): Preparation of (2-(4-(1H-pyrazol-1-yl)phenyl)-6-((((1R,2S)-2-(4-fluorophenyl)cyclopropyl)amino)methyl)pyri midin-4-yl) (4-(methylsulfonyl)piperazin-1-yl)methanone

2-(4-(1H-pyrazol-1-yl)phenyl)-6-(4-(methylsulfonyl)piperazin-1-carbonyl)pyrimidine-4-formald ehyde (100 mg, 0.23 mmol) is added to DCE (2 mL), stirred at 25° C. for 3 h, and then added with MeOH (0.5 mL), AcOH (0.1 mL) and (1R,2S)-2-(4-fluorophenyl)cyclopropyl-1-amine (51 mg, 0.23 mmol); When TLC shows that the raw materials are completely consumed, the reaction solution is added with NaBH₃CN(57 mg, 0.91 mmol), and stirred at room temperature for 2 h; and when LC-MS shows the reaction is completed, a crude product is obtained by concentration and delivered to Prep-HPLC for preparation (separation method 4) to obtain (2-(4-(1H-pyrazol-1-yl)phenyl)-6-((((1R,2S)-2-(4-fluorophenyl)cyclopropyl)amino)methyl)pyri midin-4-yl) (4-(methylsulfonyl)piperazin-1-yl)methanone with a yield of 20.2%.

¹H NMR (400 MHz, Methanol-d₄) δ ppm 8.57 (d, J=8.6 Hz, 2H), 8.34 (d, J=2.2 Hz, 1H), 7.90 (d, J=8.6 Hz, 2H), 7.78 (s, 1H), 7.56 (s, 1H), 7.02 (dd, J=8.2, 5.6 Hz, 2H), 6.91 (t, J=8.6 Hz, 2H), 6.58 (s, 1H), 4.13 (s, 2H), 4.05-3.87 (m, 2H), 3.81-3.63 (m, 2H), 3.50-3.35 (m, 4H), 2.91 (s, 3H), 2.57-2.20 (m, 1H), 2.01-1.84 (m, 1H), 1.28-0.81 (m, 2H).

ESI-MS m/z: 576.2 [M+H]⁺.

### Example 3

### Preparation of (R)-4-(1-(3-aminopiperidin-1-yl)-6-(p-tolyl)pyrrolo[1,2-a]pyrazine-7-yl)benzonitrile

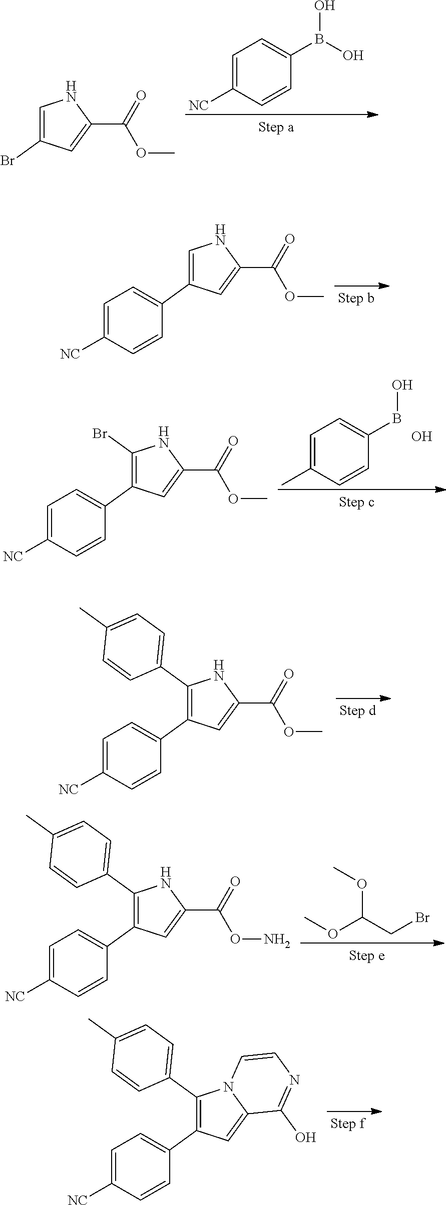

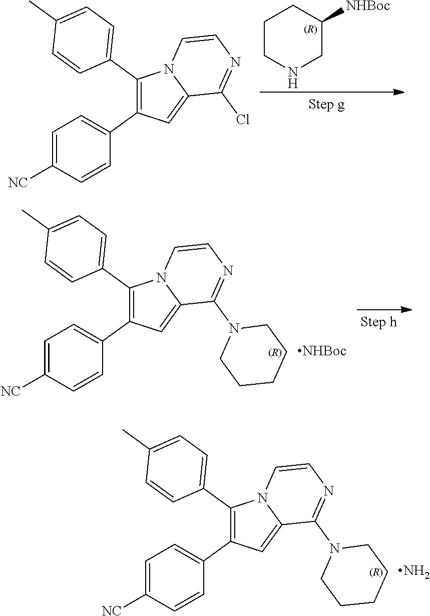

### Step a): Preparation of methyl 4-(4-cyanophenyl)-1H-pyrrole-2-carboxylate

Methyl 4-bromo-1H-pyrrole-2-carboxylate (1.0 g, 4.9 mmol), (4-cyanophenyl) boronic acid (1.1 g, 7.4 mmol), Cs₂CO₃ (4.8 g, 14.7 mmol), and Pd(dppf)Cl₂ (358 mg, 0.5 mmol) are added to 20 mL of dioxane and then added with 4 ml of water; and the mixed reaction solution is purged by nitrogen blowing and protected with nitrogen. The reaction solution reacts in a microwave reactor at 110° C. for 1 h; when LC-MS shows that the reaction is completed, a crude product is obtained by concentration, and the residue is purified by silica gel chromatography (eluent: petroleum ether/ethyl acetate=5/4) to obtain methyl 4-(4-cyanophenyl)-1H-pyrrole-2-carboxylate with a yield of 53.1%.

¹H NMR (400 MHz, DMSO-d₆) δppm 12.30 (s, 1H), 7.85 (d, J=8.4 Hz, 2H), 7.78-7.70 (m, 3H), 7.34 (d, J=1.7 Hz, 1H), 3.80 (s, 3H).

ESI-MS m/z: 227.1[M+H]⁺.

### Step b): Preparation of methyl 5-bromo-4-(4-cyanophenyl)-1H-pyrrole-2-carboxylate

Methyl 4-(4-cyanophenyl)-1H-pyrrole-2-carboxylate (4.5 g, 19.9 mmol) is dissolved in DMF (50 mL), cooled to 0° C., added with NBS (3.7 g, 20.9 mmol) in batches, and reacted at 0° C. for 1 h; when LC-MS shows that the reaction is completed, the reaction solution is quenched with 20 mL of water, and extracted with ethyl acetate (20 mL×2); organic phases are combined, washed with saturated saline (15 mL×2), dried with anhydrous sodium sulfate, filtered, and concentrated to obtain a crude product; and the residue is purified by silica gel chromatography (eluent: petroleum ether/ethyl acetate=10/9) to obtain methyl 5-bromo-4-(4-cyanophenyl)-1H-pyrrole-2-carboxylate with a yield of 60.8%.

¹H NMR (400 MHz, DMSO-d₆) δppm 13.12 (s, 1H), 7.89-7.80 (m, 4H), 7.21 (s, 1H), 3.81 (s, 3H).

ESI-MS m/z: 304.9[M+H]⁺.

### Step c): Preparation of methyl 4-(4-cyanophenyl)-5-(p-tolyl)-1H-pyrrole-2-carboxylate

Methyl 5-bromo-4-(4-cyanophenyl)-1H-pyrrole-2-carboxylate (3.7 g, 12.1 mmol), p-tolylboronic acid (2.5 g, 18.2 mmol), Na₂CO₃ (3.8 g, 36.3 mmol), and Pd(dppf)Cl₂ (880 mg, 1.2 mmol) are added to 80 mL of DMF; the mixed reaction solution is displaced with nitrogen for three times and reacted at 110° C. for 12 h; when LC-MS shows that the reaction is completed, the reaction solution is quenched with 20 mL of water, and extracted with ethyl acetate (20 mL×2); organic phases are combined, washed with saturated saline (15 mL×2), dried with anhydrous sodium sulfate, filtered, and concentrated to obtain a crude product; and the residue is purified by silica gel chromatography (eluent: dichloromethane/ethyl acetate=10/1) to obtain methyl 4-(4-cyanophenyl)-5-(p-tolyl)-1H-pyrrole-2-carboxylate with a yield of 78.5%.

¹H NMR (400 MHz, DMSO-d₆) δppm 12.31 (s, 1H), 7.79-7.66 (m, 3H), 7.42-7.36 (m, 2H), 7.25 (d, J=8.2 Hz, 2H), 7.21-7.17 (m, 2H), 3.81 (s, 3H), 2.33 (s, 3H).

ESI-MS m/z: 317.1[M+H]⁺.

### Step d): Preparation of 4-(4-cyanophenyl)-5-(p-tolyl)-1H-pyrrole-2-carboxamide

Methyl 4-(4-cyanophenyl)-5-(p-tolyl)-1H-pyrrole-2-carboxylate (1.0 g, 3.16 mmol) is dissolved in 20 mL of methanol solution of 7M ammonia, placed in a stuffy reactor, and reacted at 100° C. for 12 h; when LC-MS shows that the reaction is completed, the reaction solution is concentrated under vacuum; and the residue is purified by silica gel chromatography (eluent: dichloromethane/ethyl acetate=10/1) to obtain 4-(4-cyanophenyl)-5-(p-tolyl)-1H-pyrrole-2-carboxamide with a yield of 22.1%.

¹H NMR (400 MHz, DMSO-d₆) δppm 11.06 (s, 1H), 10.90 (d, J=1.8 Hz, 1H), 8.30 (q, J=4.2, 3.6 Hz, 2H), 8.15 (dd, J=8.4, 1.2 Hz, 2H), 7.61 (t, J=7.8 Hz, 3H), 7.25 (t, J=7.6 Hz, 1H), 7.19-7.14 (m, 1H), 2.31 (s, 3H).

ESI-MS m/z: 302.1[M+H]⁺.

### Step e): Preparation of 4-(1-hydroxy-6-(p-tolyl)pyrrolo[1,2-a]pyrazin-7-yl) benzonitrile

4-(4-cyanophenyl)-5-(p-tolyl)-1H-pyrrole-2-carboxamide (200 mg, 0.66 mmol) is dissolved in 5 mL of DMF, added with 2-bromo-1,1-dimethoxyethane (225 mg, 1.3 mmol) and Cs₂CO₃ (648 mg, 1.99 mmol), warmed to 100° C. and reacted for 24 h; when LC-MS shows that the reaction is completed, the reaction solution is quenched with 20 mL of water, and extracted with ethyl acetate (20 mL×2); organic phases are combined, washed with saturated saline (15 mL×2), dried with anhydrous sodium sulfate, filtered, and concentrated to obtain a crude product; and the residue is purified by silica gel chromatography (eluent: dichloromethane/methanol=20/1) to obtain 4-(1-hydroxy-6-(p-tolyl)pyrrolo[1,2-a]pyrazin-7-yl) benzonitrile with a yield of 60.6%.

ESI-MS m/z: 326.1[M+H]⁺.

### Step f): Preparation of 4-(1-chloro-6-(p-tolyl)pyrrolo[1,2-a]pyrazin-7-yl)benzonitrile

4-(1-hydroxy-6-(p-tolyl)pyrrolo[1,2-a]pyrazin-7-yl) benzonitrile (120 mg, 0.37 mmol) is dissolved with 1,2-dichloroethane (5 mL), and added with phosphorus oxychloride (2.5 mL), and the system is warmed to 80° C. and reacted for 8 h. When LC-MS shows that the reaction is completed, the reaction solution is concentrated under vacuum, and then the solid is dissolved with 3 mL of anhydrous dichloromethane, added with 0.2 mL of triethylamine and concentrated to obtain a crude product. The residue is purified by silica gel chromatography (eluent: petroleum ether/ethyl acetate=10/1) to obtain 4-(1-chloro-6-(p-tolyl)pyrrolo[1,2-a]pyrazin-7-yl)benzonitrile with a yield of 62.2%.

ESI-MS m/z: 344.1[M+H]⁺.

### Step g): Preparation of tert-butyl (R)-(1-(7-(4-cyanophenyl)-6-(p-tolyl)pyrrolo[1,2-a]pyrazin-1-yl)piperidin-3-yl)carbamate

4-(1—Chloro-6-(p-tolyl)pyrrolo[1,2-a]pyrazin-7-yl)benzonitrile (70 mg, 0.20 mmol) and tert-butyl (R)-piperidin-3-carbamate (60 mg, 0.30 mmol) are dissolved in 3 mL of DMSO, and added with DIPEA (103 mg, 0.80 mmol), and the system is warmed to 55° C. under nitrogen protection and reacted for 8 h. When LC-MS shows that the reaction is completed, the mixture is quenched with 20 mL of water, and extracted with ethyl acetate (20 mL×2); organic phases are combined, washed with saturated saline (15 mL×2), dried with anhydrous sodium sulfate, filtered, and concentrated to obtain a crude product; and the residue is purified by silica gel chromatography (eluent: petroleum ether/ethyl acetate=5/4) to obtain tert-butyl (R)-(1-(7-(4-cyanophenyl)-6-(p-tolyl)pyrrolo[1,2-a]pyrazin-1-yl)piperidin-3-yl)carbamate with a yield of 95.0%.

¹H NMR (400 MHz, DMSO-d₆) δppm 7.70 (d, J=7.8 Hz, 2H), 7.57-7.44 (m, 3H), 7.37 (d, J=7.6 Hz, 2H), 7.27 (d, J=7.8 Hz, 2H), 7.07 (t, J=3.8 Hz, 2H), 4.42 (d, J=12.8 Hz, 1H), 4.23 (d, J=13.0 Hz, 1H), 3.57 (s, 1H), 2.95 (t, J=11.2 Hz, 1H), 2.83 (t, J=11.2 Hz, 1H), 2.-2.35 (s, 3H), 2.03-1.75 (m, 3H), 1.61 (d, J=11.4 Hz, 1H), 1.41 (s, 9H).

ESI-MS m/z: 508.2 [M+H]⁺.

### Step h): Preparation of (R)-4-(1-(3-aminopiperidin-1-yl)-6-(p-tolyl)pyrrolo[1,2-a]pyrazin-7-yl)benzonitrile

### Tert-butyl

(R)-(1-(7-(4-cyanophenyl)-6-(p-tolyl)pyrrolo[1,2-a]pyrazin-1-yl)piperidin-3-yl)carbamate (90 mg, 0.18 mmol) is dissolved with 5 mL of ethyl acetate, added with 5 mL of 4M HCl(ethyl acetate solution), and reacted at room temperature for 1 h; a large number of solids are generated in the reaction system; and when LC-MS shows that the reaction is completed, a crude product is obtained by concentration and delivered to Prep-HPLC for preparation (separation method 4) to obtain (R)-4-(1-(3-aminopiperidin-1-yl)-6-(p-tolyl)pyrrolo[1,2-a]pyrazin-7-yl)benzonitrile with a yield of 72.2%.

¹H NMR (400 MHz, DMSO-d₆) δ ppm 7.79 (d, J=7.8 Hz, 3H), 7.56 (d, J=8.2 Hz, 2H), 7.46-7.36 (m, 3H), 7.29 (d, J=7.8 Hz, 2H), 7.05 (d, J=5.6 Hz, 1H), 4.56 (d, J=13.0 Hz, 1H), 4.16 (d, J=13.2 Hz, 1H), 3.66 (s, 3H), 2.42 (s, 3H), 2.15 (d, J=6.4 Hz, 1H), 1.97 (s, 1H), 1.77 (t, J=8.4 Hz, 2H).

ESI-MS m/z: 408.2 [M+H]⁺.

### Example 4

### Preparation of (R)-4-(1-(3-aminopiperidin-1-yl)-6-(p-tolyl)pyrrolo[1,2-d] [1,2,4]triazine-7-yl)benzonitrile

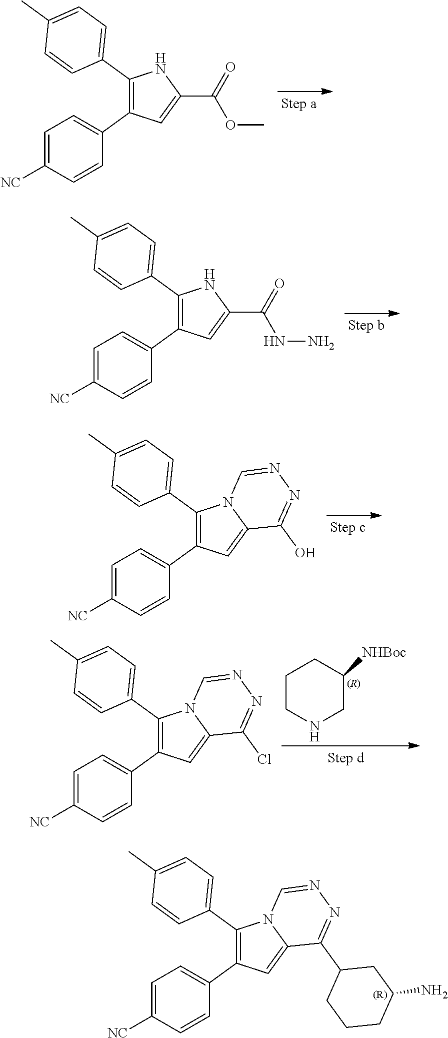

### Step a): Preparation of 4-(4-cyanophenyl)-5-(p-tolyl)-1H-pyrrole-2-carbohydrazide

Methyl 4-(4-cyanophenyl)-5-(p-tolyl)-1H-pyrrole-2-carboxylate (1.0 g, 3.16 mmol) is dissolved in hydrazine hydrate and ethanol (in the ratio of 1:1, 10 mL), placed in a stuffy reactor, and reacted at 80° C. for 5 h; when LC-MS shows that the reaction is completed, a crude product is obtained by concentration; and the residue is purified by silica gel chromatography (eluent: dichloromethane/methanol=20/1) to obtain 4-(4-cyanophenyl)-5-(p-tolyl)-1H-pyrrole-2-carbohydrazide with a yield of 64.9%.

¹H NMR (400 MHz, DMSO-d₆) δppm 9.36 (s, 1H), 7.74-7.69 (m, 2H), 7.34 (d, J=8.2 Hz, 2H), 7.24 (d, J=7.8 Hz, 2H), 7.17 (d, J=7.8 Hz, 2H), 7.03 (d, J=2.6 Hz, 1H), 4.46 (s, 2H), 3.17 (d, J=5.2 Hz, 1H), 2.32 (s, 3H).

ESI-MS m/z: 317.1 [M+H]⁺.

### Step b): Preparation of 4-(1-hydroxy-6-(p-tolyl)pyrrolo[1,2-d] [1,2,4] triazin-7-yl)benzonitrile

4-(4-cyanophenyl)-5-(p-tolyl)-1H-pyrrole-2-carbohydrazide (650 mg, 2.05 mmol) is dissolved in triethyl orthoformate (5 mL) and refluxed for 1 h; and when LC-MS shows that the reaction is completed, a crude product is obtained by vacuum concentration. The crude product is then dissolved with 15 mL of absolute ethanol, added with potassium hydroxide (460 mg, 8.2 mmol), refluxed for 1 h, and concentrated to obtain a crude product; and the residue is purified by silica gel chromatography (eluent: petroleum ether/ethyl acetate=1/1) to obtain 4-(1-hydroxy-6-(p-tolyl)pyrrolo[1,2-d] [1,2,4] triazin-7-yl)benzonitrile with a yield of 29.7%.

ESI-MS m/z: 327.1[M+H]⁺.

### Step c): Preparation of 4-(1-chloro-6-(p-tolyl)pyrrolo[1,2-d][1,2,4]triazin-7-yl)benzonitrile

4-(1-hydroxy-6-(p-tolyl)pyrrolo[1,2-d] [1,2,4]triazin-7-yl)benzonitrile (200 mg, 0.61 mmol) is dissolved with 1,2-dichloroethane (5 mL) and added with phosphorus oxychloride (2.5 mL); the system is warmed to 75° C. and reacted for 3 h; when alkaline LC-MS shows that the reaction is completed, the reaction solution is concentrated under vacuum; the solid is then dissolved with 3 mL of anhydrous dichloromethane, added with 0.2 mL of triethylamine, and concentrated to obtain a crude product; and the residue is purified by silica gel chromatography (eluent: petroleum ether/ethyl acetate=10/1) to obtain 4-(1-chloro-6-(p-tolyl)pyrrolo[1,2-d][1,2,4]triazin-7-yl)benzonitrile with a yield of 37.7%.

ESI-MS m/z: 345.1[M+H]⁺.

### Step d): Preparation of (R)-4-(1-(3-aminopiperidin-1-yl)-6-(p-tolyl)pyrrolo[1,2-d][1,2,4]triazin-7-yl)benzonitrile

4-(1-chloro-6-(p-tolyl)pyrrolo[1,2-d] [1,2,4]triazin-7-yl)benzonitrile (80 mg, 0.23 mmol) and tert-butyl (R)-piperidin-3-yl-carbamate (93.1 mg, 0.47 mmol) are dissolved in 3 mL of DMSO, and added with DIPEA (90.2 mg, 0.7 mmol), and the system is warmed to 55° C. under nitrogen protection and reacted for 3 h. When the alkaline LC-MS shows that the reaction is completed, a crude product is obtained by concentration; and the residue is purified by silica gel chromatography (eluent: petroleum ether/ethyl acetate=10/3) to obtain tert-butyl (R)-(1-(7-(4-cyanophenyl)-6-(p-tolyl)pyrrolo[1,2-d][1,2,4]triazin-1-yl)piperidin-3-yl)carbamate. Tert-butyl (R)-(1-(7-(4-cyanophenyl)-6-(p-tolyl)pyrrolo[1,2-d][1,2,4]triazin-1-yl)piperidin-3-yl)carbamate (80 mg, 0.16 mmol) is dissolved with 3 mL of 4M HCl(ethyl acetate solution), and reacted at room temperature for 30 min. When the alkaline LC-MS shows that the reaction is completed, a crude product is obtained by concentration and delivered to Prep-HPLC for preparation (separation method 4) to obtain (R)-4-(1-(3-aminopiperidin-1-yl)-6-(p-tolyl)pyrrolo[1,2-d] [1,2,4]triazin-7-yl)benzonitrile with a yield of 8.3%.

¹H NMR (400 MHz, Methanol-d₄) δ ppm 8.55 (d, J=16.4 Hz, 2H), 7.67-7.62 (m, 2H), 7.54-7.49 (m, 2H), 7.38 (d, J=7.8 Hz, 2H), 7.30 (d, J=7.8 Hz, 3H), 4.33-4.24 (m, 1H), 4.12 (d, J=13.4 Hz, 1H), 3.52-3.44 (m, 2H), 3.39 (dd, J=12.8, 8.4 Hz, 1H), 2.44 (s, 3H), 2.19 (d, J=11.6 Hz, 1H), 1.99 (d, J=16.0 Hz, 1H), 1.82 (dd, J=9.6, 4.0 Hz, 1H), 1.79-1.70 (m, 1H).

ESI-MS m/z: 409.2 [M+H]⁺.

### Example 5

### Preparation of (R)-4-(8-(3-aminopiperidin-1-yl)-3-(p-tolyl)imidazo[1,2-a]pyrazin-2-yl)benzonitrile hydrochloride

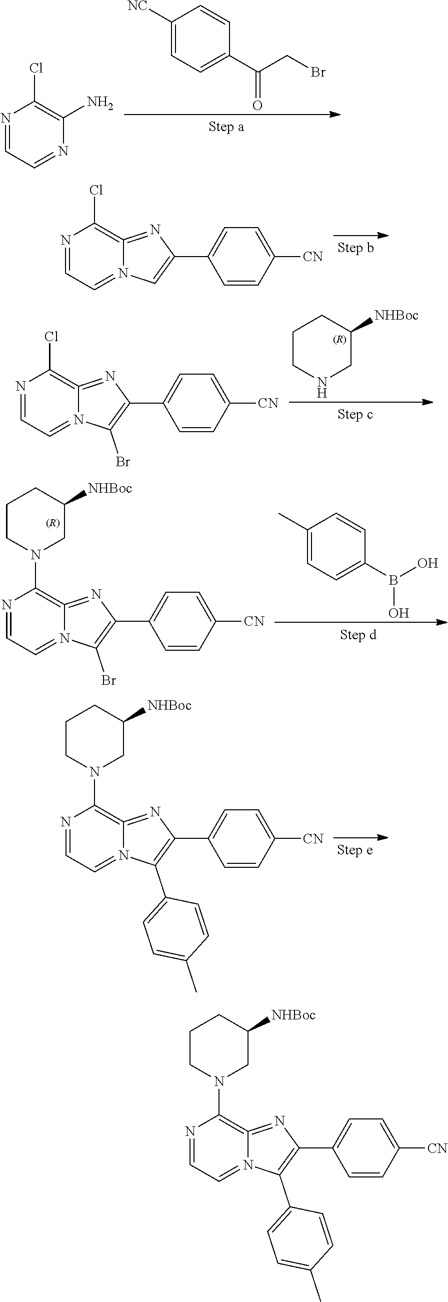

### Step a:) Preparation of 4-(8-chloroimidazolo[1,2-a]pyrazin-2-yl)benzonitrile

3-chloropyrazin-2-amine (1 g, 4.6 mmol), 4-(2-bromoacetyl)benzonitrile (1 g, 4.6 mmol), diethylaniline (2.1 g, 13.9 mmol), and DMSO (10 mL) are added to a reaction flask sequentially, warmed to 130° C. and reacted overnight. When the reaction is completed, the reaction solution is cooled to room temperature, then added with water (50 mL), and extracted with ethyl acetate (20 mL×2); organic phases are combined, washed with saturated saline (100 mL×2), dried with anhydrous sodium sulfate, and filtered; the organic phase is concentrated under a reduced pressure to dryness; and the residue is purified by silica gel chromatography (eluent: petroleum ether/ethyl acetate=2/1) to obtain 4-(8-chloroimidazolo[1,2-a]pyrazin-2-yl)benzonitrile with a yield of 30.5%.

ESI-MS m/z=255.1 [M+H]⁺.

### Step b): Preparation of 4-(3-bromo-8-chloroimidazolo[1,2-a]pyrazin-2-yl)benzonitrile

4-(8-chloroimidazolo[1,2-a]pyrazin-2-yl)benzonitrile (310 mg, 1.2 mmol) and NBS (233 mg, 1.32 mmol) are dissolved in DMF (5 mL) and reacted for 2 h in an ice bath while stirring. When the reaction is completed, the reaction solution is added with water (50 mL), and extracted with ethyl acetate (20 mL×2); organic phase is then washed with saturated saline (20 mL), dried with anhydrous sodium sulfate, filtered, and concentrated under a reduced pressure to obtain 4-(3-bromo-8-chloroimidazolo[1,2-a]pyrazin-2-yl)benzonitrile with a yield of 75.0%.

ESI-MS m/z=334.2 [M+H]⁺.

### Step c): Preparation of tert-butyl (R)-(1-(3-bromo-2-(4-cyanophenyl)imidazolo[1,2-a]pyrazin-8-yl)piperidin-3-yl)carbamate

4-(3-bromo-8-chloroimidazolo[1,2-a]pyrazin-2-yl)benzonitrile (300 mg, 0.90 mmol), tert-butyl (R)-piperidin-3-yl-carbamate (263 mg, 0.90 mmol), and DIPEA (348 mg, 2.7 mmol) are dissolved in DMSO (5 mL), and the reaction is warmed to 55° C. for 2 h. When the reaction is completed, the reaction solution is cooled to room temperature, then added with water (50 mL), and extracted with ethyl acetate (20 mL×2); organic phases are combined, washed with saturated saline (20 mL), dried with anhydrous sodium sulfate, filtered, and concentrated to obtain a crude product; and the residue is purified by silica gel chromatography (eluent: petroleum ether/ethyl acetate=2/1) to obtain tert-butyl (R)-(1-(3-bromo-2-(4-cyanophenyl)imidazolo[1,2-a]pyrazin-8-yl)piperidin-3-yl)carbamate with a yield of 35.0%.

ESI-MS m/z=498.1 [M+H]⁺.

### Step d): Preparation of tert-butyl (R) (1-(2-(4-cyanophenyl)-3-(p-tolyl)imidazolo[1,2-a]pyrazin-8-yl)piperidin-3-yl)carbamate

### Tert-butyl

(R)-(1-(3-bromo-2-(4-cyanophenyl)imidazolo[1,2-a]pyrazin-8-yl)piperidin-3-yl)carbamate (150 mg, 0.30 mmol), p-tolylboronic acid (61.2 mg, 0.45 mmol), Cs₂CO₃ (295 mg, 0.90 mmol), Pd(dppf)Cl₂ (44.2 mg, 0.06 mmol), 1.4-dioxane (5 mL) and water (1 mL) are sequentially added to a reaction flask, nitrogen replacement is performed for three times, and the reaction is warmed to 100° C. and reacted for 3 h. When the reaction is completed, the reaction solution is cooled to room temperature, then added with water (50 mL), and extracted with ethyl acetate (20 mL×2); organic phase is then washed with saturated saline (20 mL), dried with anhydrous sodium sulfate, filtered, and concentrated to obtain a crude product; and the residue is purified by silica gel chromatography (eluent: petroleum ether/ethyl acetate=1/1) to obtain tert-butyl (R)-(1-(2-(4-cyanophenyl)-3-(p-tolyl)imidazolo[1,2-a]pyrazin-8-yl)piperidin-3-yl)carbamate with a yield of 56.0%.

¹H NMR (400 MHz, DMSO-d₆) δppm 7.76 (d, J=2.8 Hz, 4H), 7.44 (d, J=8.0 Hz, 2H), 7.39 (d, J=7.8 Hz, 2H), 7.33 (d, J=4.6 Hz, 1H), 7.25 (d, J=4.6 Hz, 1H), 6.95 (d, J=7.8 Hz, 1H), 5.31 (s, 1H), 5.09 (s, 1H), 3.21 (d, J=11.8 Hz, 2H), 2.44 (s, 4H), 1.87 (d, J=32.6 Hz, 2H), 1.55 (q, J=9.4 Hz, 2H), 1.39 (s, 9H).

ESI-MS m/z=509.1 [M+H]⁺.

### Step e):Preparation of (R)-4-(8-(3-aminopiperidin-1-yl)-3-(p-tolyl)imidazolo[1,2-a]pyrazin-2-yl)benzonitrile hydrochloride

### Tert-butyl

(R)-(1-(2-(4-cyanophenyl)-3-(p-tolyl)imidazolo[1,2-a]pyrazin-8-yl)piperidin-3-yl)carbamate (90 mg, 0.17 mmol) is dissolved in 5 mL of 4M HCl (ethyl acetate solution, 5 mL) and reacted for 1 h at room temperature. When the reaction is completed, solids are generated in the reaction solution, and filtered; a filter cake is collected and dried to obtain (R)-4-(8-(3-aminopiperidin-1-yl)-3-(p-tolyl)imidazolo[1,2-a]pyrazin-2-yl)benzonitrile hydrochloride with a yield of 82.3%.

¹H NMR (400 MHz, DMSO-d₆) δ ppm 8.34 (d, J=5.4 Hz, 3H), 7.81 (s, 4H), 7.46 (d, J=7.8 Hz, 2H), 7.41-7.35 (m, 4H), 5.09 (d, J=65.2 Hz, 2H), 3.38 (s, 1H), 2.45 (s, 3H), 2.12 (d, J=11.2 Hz, 1H), 1.97-1.86 (m, 1H), 1.80-1.66 (m, 2H).

ESI-MS m/z: 409.2 [M+H]⁺.

### Example 6

### Preparation of 4-(5-(3-amino-8-azabicyclo[3.2.1]octane-8-carbonyl)-2-(2-fluoro-4-methylphenyl)thiophen-3-yl)-2-fluorobenzonitrile hydrochloride

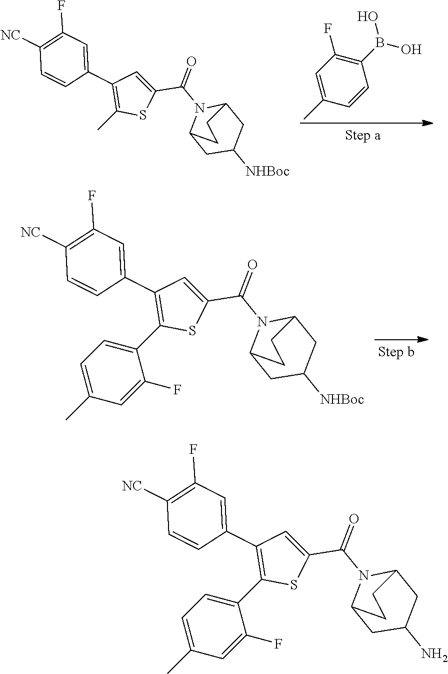 [SMILES: Cc1sc(C(=O)N2C3CCC2CC(NC(=O)OC(C)(C)C)C3)cc1-c1ccc(C#N)c(F)c1 ; Cc1ccc(B(O)O)c(F)c1 ; Cc1ccc(-c2sc(C(=O)N3C4CCC3CC(NC(=O)OC(C)(C)C)C4)cc2-c2ccc(C#N)c(F)c2)c(F)c1 ; Cc1ccc(-c2sc(C(=O)N3C4CCC3CC(N)C4)cc2-c2ccc(C#N)c(F)c2)c(F)c1]

### Step a): Preparation of tert-butyl (8-(4-(4-cyano-3-fluorophenyl)-5-(2-fluoro-4-methylphenyl)thiophen-2-carbonyl)-8-azabicyclo[3.2.1]octane-3-yl)carbamate

### Tert-butyl

(8-(5-bromo-4-(4-cyano-3-fluorophenyl)thiophen-2-carbonyl)-8-azabicyclo[3.2.1]octane-3-yl)ca rbamate (600 mg, 0.938 mmol), (2-fluoro-4-methylphenyl)boronic acid (173 mg, 1.13 mmol), K₂CO₃ (915 mg, 2.81 mmol), and Pd(dppf)Cl₂ (69 mg, 0.09 mmol) are dissolved in 1,4-dioxane (8.4 mL) and added with water (1.6 mL); and the mixed reaction solution is purged by nitrogen blowing and protected with nitrogen. A reaction is performed in a microwave reactor at 100° C. for 1 h. When the reaction is completed, the mixture is extracted with ethyl acetate (20 mL×2); organic phase is then washed with saline (50 mL), dried with anhydrous sodium sulfate, filtered, and concentrated to obtain a crude product. The residue is purified by silica gel chromatography (eluent: petroleum ether/ethyl acetate=1/1) to obtain tert-butyl (8-(4-(4-cyano-3-fluorophenyl)-5-(2-fluoro-4-methylphenyl)thiophen-2-carbonyl)-8-azabicyclo[3.2.1]octane-3-yl)carbamate with a yield of 43.7%.

¹H NMR (400 MHz, Chloroform-d) δ ppm 7.55-7.46 (m, 2H), 7.19 (tt, J=7.6, 2.8 Hz, 1H), 7.14-7.04 (m, 2H), 6.98 (t, J=5.8 Hz, 1H), 6.92-6.83 (m, 1H), 4.80 (s, 2H), 4.45-4.33 (m, 1H), 4.12 (tt, J=9.4, 6.0 Hz, 1H), 2.38 (t, J=3.2 Hz, 3H), 2.21-2.02 (m, 4H), 1.89 (s, 2H), 1.69-1.56 (m, 2H), 1.43 (s, 9H).

ESI-MS m/z: 564.2[M+H]⁺.

### Step b): Preparation of 4-(5-(3-amino-8-azabicyclo[3.2.1]octane-8-carbonyl)-2-(2-fluoro-4-methylphenyl)thiophen-3-yl)-2-fluorobenzonitrile hydrochloride

### Tert-butyl

(8-(4-(4-cyano-3-fluorophenyl)-5-(2-fluoro-4-methylphenyl)thiophen-2-carbonyl)-8-azabicyclo[3.2.1]octane-3-yl)carbamate (50 mg, 0.09 mmol) is dissolved in 1M HCl (ethyl acetate solution, 1.5 mmol, 2 mL) at room temperature and stirred for 2 h under nitrogen protection; when the raw materials are completely converted, a white solid precipitated; the reaction solution is filtered to obtain a filter cake; the filter cake is washed with ethyl acetate (5 mL), and dried to obtain a white solid 4-(5-(3-amino-8-azabicyclo[3.2.1]octane-8-carbonyl)-2-(2-fluoro-4-methylphenyl)thiophen-3-yl)-2-fluorobenzonitrile hydrochloride with a yield of 66.7%.

¹H NMR (400 MHz, Methanol-d₄) δppm 7.72 (d, J=2.0 Hz, 1H), 7.67 (t, J=7.4 Hz, 1H), 7.29 (t, J=7.4 Hz, 2H), 7.23 (d, J=8.2 Hz, 1H), 7.10 (d, J=7.8 Hz, 1H), 7.00 (d, J=11.2 Hz, 1H), 4.90-4.89 (m, 2H), 3.78 (dt, J=12.4, 6.4 Hz, 1H), 2.40 (d, J=2.4 Hz, 3H), 2.15 (d, J=12.8 Hz, 4H), 1.98-1.81 (m, 4H).

ESI-MS m/z: 464.2[M+H]⁺.

### Example 7

### Preparation of 4-(5-((3-amino-8-azabicyclo[3.2.1]octane-8-yl)methyl)-2-(2-fluoro-4-methylphenyl)thiophen-3-yl)-2-fluorobenzonitrile trifluoroacetate

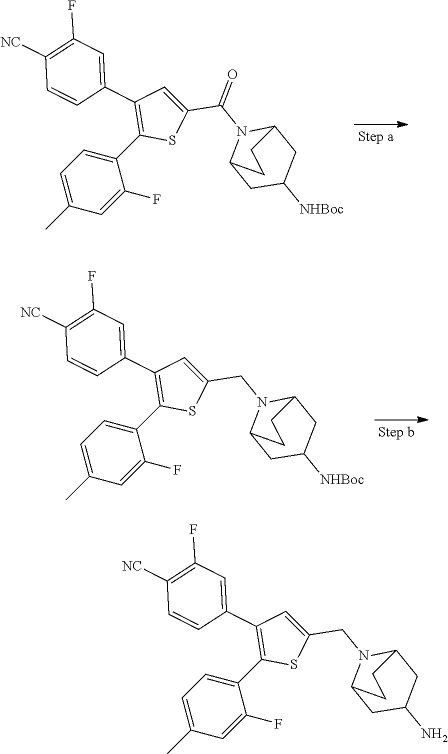 [SMILES: Cc1ccc(-c2sc(C(=O)N3C4CCC3CC(NC(=O)OC(C)(C)C)C4)cc2-c2ccc(C#N)c(F)c2)c(F)c1 ; Cc1ccc(-c2sc(CN3C4CCC3CC(NC(=O)OC(C)(C)C)C4)cc2-c2ccc(C#N)c(F)c2)c(F)c1 ; Cc1ccc(-c2sc(CN3C4CCC3CC(N)C4)cc2-c2ccc(C#N)c(F)c2)c(F)c1]

### Step a): Preparation of tert-butyl (8-((4-(4-cyano-3-fluorophenyl)-5-(2-fluoro-4-methylphenyl)thiophen-2-yl)methyl)-8-azabicycl o[3.2.1]octane-3-yl)carbamate

### Tert-butyl

(8-(4-(4-cyano-3-fluorophenyl)-5-(2-fluoro-4-methylphenyl)thiophen-2-carbonyl)-8-azabicyclo[3.2.1]octane-3-yl)carbamate (175 mg, 0.30 mmol) is dissolved in anhydrous acetonitrile (10 mL), added with 2M borane dimethyl sulfide solution (138 mg, 1.81 mmol, 0.9 mL), and protected under nitrogen; and the mixed reaction solution is warmed to 50° C., and reacted for 16 h to complete the reaction. The reaction solution is quenched with methanol and concentrated under vacuum to remove tetrahydrofuran; the residue is purified by silica gel chromatography (eluent: petroleum ether/ethyl acetate=1/1) to obtain tert-butyl (8-((4-(4-cyano-3-fluorophenyl)-5-(2-fluoro-4-methylphenyl)thiophen-2-yl)methyl)-8-azabicycl o[3.2.1]octane-3-yl)carbamate with a yield of 36.7%.

¹H NMR (400 MHz, Chloroform-d) δ ppm 7.46 (t, J=7.4 Hz, 1H), 7.17 (t, J=7.8 Hz, 1H), 7.07 (t, J=9.4 Hz, 2H), 7.00-6.91 (m, 2H), 6.85 (d, J=10.8 Hz, 1H), 4.36 (d, J=8.6 Hz, 1H), 3.73 (s, 2H), 3.34 (s, 1H), 2.37 (s, 3H), 2.04-1.96 (m, 2H), 1.91-1.81 (m, 2H), 1.74 (d, J=8.4 Hz, 2H), 1.52 (t, J=12.8 Hz, 2H), 1.43 (s, 9H), 1.26 (q, J=7.8, 6.6 Hz, 2H).

ESI-MS m/z: 550.2[M+H]⁺.

### Step b): Preparation of 4-(5-((3-amino-8-azabicyclo[3.2.1]octane-8-yl)methyl)-2-(2-fluoro-4-methylphenyl)thiophen-3-yl)-2-fluorobenzonitrile trifluoroacetate

### Tert-butyl

(8-((4-(4-cyano-3-fluorophenyl)-5-(2-fluoro-4-methylphenyl)thiophen-2-yl)methyl)-8-azabicycl o[3.2.1]octane-3-yl)carbamate (30 mg, 0.05 mmol) is dissolved in a TFA/DCM solution (1:3, 2.5 mL); and the reaction solution is stirred at room temperature under nitrogen protection, and reacted for 30 min to complete the reaction. The reaction solution is concentrated to obtain a solid and freeze-dried to obtain 4-(5-((3-amino-8-azabicyclo[3.2.1]octane-8-yl)methyl)-2-(2-fluoro-4-methylphenyl)thiophen-3-yl)-2-fluorobenzonitrile trifluoroacetate with a yield of 80.0%.

¹H NMR (400 MHz, Methanol-d₄) δ ppm 7.69-7.55 (m, 2H), 7.30-7.17 (m, 3H), 7.08 (d, J=7.8 Hz, 1H), 6.98 (d, J=11.0 Hz, 1H), 4.59 (s, 2H), 4.20 (d, J=5.2 Hz, 2H), 3.78 (dq, J=11.2, 5.8, 4.8 Hz, 1H), 2.59-2.43 (m, 2H), 2.38 (d, J=2.0 Hz, 3H), 2.31-2.11 (m, 6H), 1.28 (s, 2H).

ESI-MS m/z: 450.2[M+H]⁺.

### Example 8

### Preparation of 4-(5-(3-amino-8-azabicyclo[3.2.1]octane-8-carbonyl)-2-(5-fluoro-3-pentylbenzo[d]isoxazol-6-yl) thiophen-3-yl)-2-fluorobenzonitrile

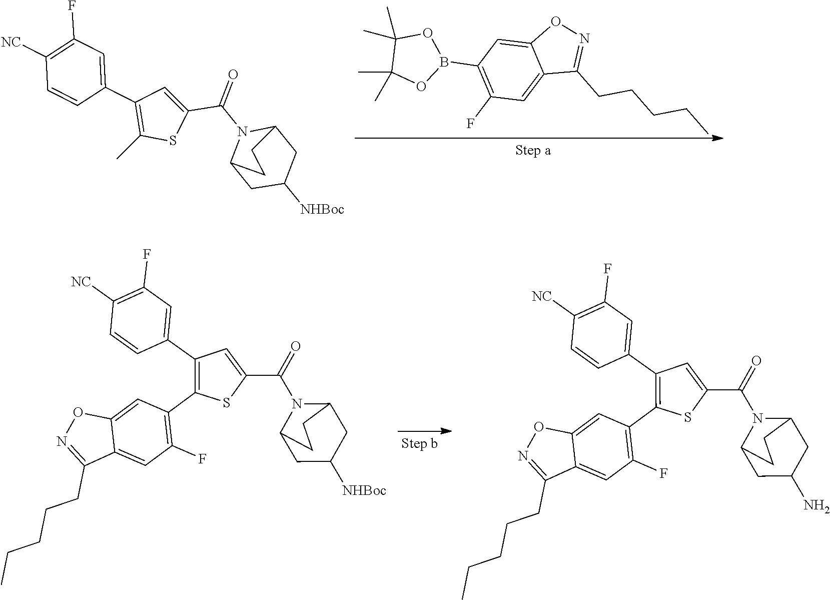 [SMILES: Cc1sc(C(=O)N2C3CCC2CC(NC(=O)OC(C)(C)C)C3)cc1-c1ccc(C#N)c(F)c1 ; CCCCCc1noc2cc(B3OC(C)(C)C(C)(C)O3)c(F)cc12 ; CCCCCc1noc2cc(-c3sc(C(=O)N4C5CCC4CC(NC(=O)OC(C)(C)C)C5)cc3-c3ccc(C#N)c(F)c3)c(F)cc12 ; CCCCCc1noc2cc(-c3sc(C(=O)N4C5CCC4CC(N)C5)cc3-c3ccc(C#N)c(F)c3)c(F)cc12]

### Step a): Preparation of tert-butyl(8-(4-(4-cyano-3-fluorophenyl)-5-(5-fluoro-3-pentylbenzo[d]isoxazol-6-yl)thiophen-2-carbonyl)-8-azabicyclo[3.2.1]octane-3-yl)carbamate

### Tert-butyl

(8-(5-bromo-4-(4-cyano-3-fluorophenyl)thiophen-2-carbonyl)-8-azabicyclo[3.2.1]octane-3-yl)ca rbamate (128 mg, 0.24 mmol), 5-fluoro-3-pentyl-6-(4,4,5,5-tetramethyl-1,3,2-dioxaborolane-2-yl)benzo[d]isoxazole (100 mg, 0.36 mmol), Na₂CO₃ (51 mg, 0.482 mmol), and Pd(dppf)Cl₂ (35.2 mg, 0.05 mmol) are added to a 10 mL microwave-dedicated reaction tube, and added with 1,4-dioxane (4 mL) and water (0.8 mL) for dissolving; and the mixed solution is purged by nitrogen blowing and protected with nitrogen, and reacted for 30 min at 100° C. in the microwave reactor. When the reaction is completed, the mixture is cooled to room temperature, added with water (30 mL), and extracted with ethyl acetate (20 mL×2); organic phase is then washed with saline (10 mL), dried with anhydrous sodium sulfate, filtered, and concentrated to obtain a crude product. The residue is purified by silica gel chromatography (eluent: petroleum ether/ethyl acetate=1/1) to obtain tert-butyl (8-(4-(4-cyano-3-fluorophenyl)-5-(5-fluoro-3-pentylbenzo[d]isoxazol-6-yl)thiophen-2-carbonyl)-8-azabicyclo[3.2.1]octane-3-yl)carbamate with a yield of 25.4%.

¹H NMR (400 MHz, DMSO-d₆) δppm 7.98 (d, J=5.4 Hz, 1H), 7.92-7.74 (m, 3H), 7.63 (d, J=10.4 Hz, 1H), 7.21 (d, J=8.2 Hz, 1H), 6.79 (d, J=8.0 Hz, 1H), 4.71 (d, J=41.8 Hz, 2H), 3.90 (s, 1H), 2.95 (q, J=7.8 Hz, 2H), 2.13-1.73 (m, 8H), 1.65-1.54 (m, 2H), 1.36 (s, 9H), 1.33-1.19 (m, 4H), 0.86 (d, J=6.6 Hz, 3H).

ESI-MS m/z: 661.2[M+H]⁺.

### Step b): Preparation of 4-(5-(3-amino-8-azabicyclo[3.2.1]octane-8-carbonyl)-2-(5-fluoro-3-pentylbenzo[d]isoxazol-6-yl) thiophen-3-yl)-2-fluorobenzonitrile

### Tert-butyl

(8-(4-(4-cyano-3-fluorophenyl)-5-(5-fluoro-3-pentylbenzo[d]isoxazol-6-yl)thiophen-2-carbonyl)-8-azabicyclo[3.2.1]octane-3-yl)carbamate (40 mg, 0.06 mmol) is dissolved in 1 mL of TFA/DCM (1:3) solution under nitrogen protection; and the reaction solution is reacted for 1 h at room temperature. When the reaction is completed, the reaction solution is concentrated to obtain a crude product; the crude product is purified by Prep-HPLC (separation method 4) to obtain a white solid, i.e., 4-(5-(3-amino-8-azabicyclo[3.2.1]octane-8-carbonyl)-2-(5-fluoro-3-pentylbenzo[d]isoxazol-6-yl) thiophen-3-yl)-2-fluorobenzonitrile with a yield of 36.7%.

¹H NMR (400 MHz, DMSO-d₆) δ ppm 8.40 (s, 1H), 7.93 (d, J=5.4 Hz, 1H), 7.85-7.74 (m, 3H), 7.54 (d, J=10.4 Hz, 1H), 7.21 (d, J=8.0 Hz, 1H), 4.72 (d, J=37.4 Hz, 2H), 2.95 (t, J=7.4 Hz, 2H), 1.99 (d, J=36.8 Hz, 4H), 1.74 (q, J=7.8 Hz, 4H), 1.58 (t, J=11.8 Hz, 2H), 1.30 (h, J=3.6 Hz, 4H), 0.82 (s, 3H).

ESI-MS m/z: 561.2[M+H]⁺.

### Example 9

### Preparation of 4-(5-(3-amino-8-azabicyclo[3.2.1]octane-8-carbonyl)-2-(5-fluoro-3-pentylbenzo[d]isoxazol-6-yl) thiophen-3-yl)-2-fluorobenzonitrile formate

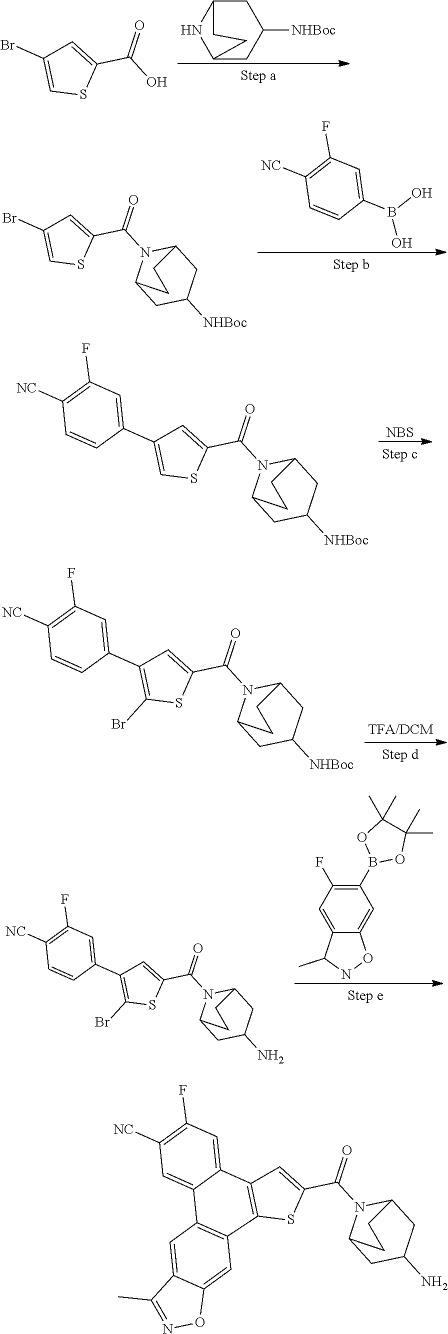

### Step a): Preparation of tert-butyl (8-(4-bromothiophene-2-carbonyl)-8-azabicyclo[3.2.1]octane-3-yl)carbamate

4-Bromothiophene-2-carboxylic acid (2.0 g, 9.71 mmol) is dissolved in DMF (25 mL), added with HATU (5.53 g, 14.56 mmol), stirred at room temperature under nitrogen protection and reacted for 0.5 h, then added with DIPEA (3.76 g, 29.12 mmol) and tert-butyl (8-azabicyclo[3.2.1]octane-3-yl)carbamate (2.41 g, 10.68 mmol), and reacted overnight at room temperature under nitrogen protection. When LC-MS shows that the reaction is completed, the reaction solution is quenched with water (60 mL), and extracted with ethyl acetate (50 mL×3); organic phase is then washed with saline (50 mL), dried with anhydrous sodium sulfate, filtered, and concentrated under vacuum. The residue is purified by silica gel chromatography (eluent: petroleum ether/ethyl acetate=2/1) to obtain tert-butyl (8-(4-bromothiophene-2-carbonyl)-8-azabicyclo[3.2.1]octane-3-yl)carbamate with a yield of 87.0%. 1H NMR (400 MHz, Chloroform-d) δ ppm 7.36 (d, J=1.4 Hz, 1H), 7.27 (d, J=1.4 Hz, 1H), 4.70 (d, J=63.6 Hz, 2H), 4.37 (d, J=8.6 Hz, 1H), 4.11 (s, 1H), 2.03 (s, 4H), 1.87 (s, 2H), 1.59 (s, 2H), 1.43 (s, 9H).

ESI-MS m/z: 415.1 [M+H]⁺.

### Step b): Preparation of tert-butyl (8-(4-(4-cyano-3-fluorophenyl)thiophen-2-carbonyl)-8-azabicyclo[3.2.1]octane-3-acyl)carbamat e

Tert-butyl (8-(4-bromothiophene-2-carbonyl)-8-azabicyclo[3.2.1]octane-3-yl)carbamate (3.50 g, 8.43 mmol), (4-cyano-3-fluorophenyl)boronic acid (1.93 g, 11.71 mmol), Cs₂CO₃ (9.51 g, 29.27 mmol), and Pd(dppf)Cl₂ (716 mg, 0.98 mmol) are added to a 30 mL microwave-dedicated reaction tube, and added with 1,4-dioxane (15 mL) and water (3 mL) for dissolving; and the mixed solution is purged by nitrogen blowing and protected with nitrogen. The reaction solution is reacted for 35 min at 120° C. in the microwave reactor; when TLC and LC-MS show that the reaction is completed, the reaction solution is added with water (60 mL), and extracted with ethyl acetate (3×20 mL); organic phases are combined and then washed with saturated saline (15 mL), dried with anhydrous sodium sulfate, filtered, and concentrated under vacuum; and the residue is purified by silica gel chromatography (eluent: ethyl acetate/petroleum ether=1/1) to obtain tert-butyl (8-(4-(4-cyano-3-fluorophenyl)thiophen-2-carbonyl)-8-azabicyclo[3.2.1]octane-3-acyl)carbamat e with a yield of 93.6%.

¹H NMR (400 MHz, Chloroform-d) δppm 7.36 (d, J=1.4 Hz, 2H), 7.27 (d, J=1.4 Hz, 3H), 4.71 (s, 2H), 4.37 (d, J=8.6 Hz, 1H), 4.12 (q, J=7.2 Hz, 1H), 2.07-2.02 (m, 4H), 1.87 (s, 2H), 1.59 (s, 2H), 1.44 (s, 9H).

ESI-MS m/z: 456.2 [M+H]⁺.

### Step c): Preparation of tert-butyl (8-(5-bromo-4-(4-cyano-3-fluorophenyl)thiophen-2-carbonyl)-8-azabicyclo[3.2.1]octane-3-ylcar bamate

### Tert-butyl

(8-(4-(4-cyano-3-fluorophenyl)thiophen-2-carbonyl)-8-azabicyclo[3.2.1]octane-3-acyl)carbamat e (3.6 g, 7.89 mmol) is dissolved in dry DMF (20 mL), added with NBS (1.5 g, 8.44 mmol), then warmed to 60° C., and reacted for 6 h under nitrogen protection. When LC-MS shows that the reaction is completed, the reaction solution is added with 50 mL of water and extracted with ethyl acetate (20 mL×3). Organic layers are combined and then washed with saline (10 mL), dried with anhydrous sodium sulfate, filtered, and concentrated under vacuum; and the residue is purified by silica gel chromatography (eluent: ethyl acetate/dichloromethane=1: 2) to obtain tert-butyl (8-(5-bromo-4-(4-cyano-3-fluorophenyl)thiophen-2-carbonyl)-8-azabicyclo[3.2.1]octane-3-yl-ca rbamate with a yield of 44.3%.

¹H NMR (400 MHz, Methanol-d₄) δppm 7.87 (t, J=7.4 Hz, 1H), 7.65 (dd, J=17.0, 9.2 Hz, 2H), 7.54 (s, 1H), 4.79-4.70 (m, 2H), 4.09-3.99 (m, 1H), 1.98 (d, J=24.8 Hz, 4H), 1.63 (t, J=12.6 Hz, 4H), 1.43 (s, 9H).

ESI-MS m/z: 534.1 [M+H]⁺.

### Step d): Preparation of 4-(5-(3-amino-8-azabicyclo[3.2.1]octane-8-carbonyl)-2-bromothiophen-3-yl)-2-fluorobenzonitril e

### Tert-butyl

(8-(5-bromo-4-(4-cyano-3-fluorophenyl)thiophen-2-carbonyl)-8-azabicyclo[3.2.1]octane-3-yl-ca rbamate (200 mg, 0.37 mmol) is dissolved in 2 mL of a TFA/DCM (1: 3) solution, reacted for 24-41 h at room temperature under nitrogen protection, and detected by TLC and LC-MS. When the reaction is completed, the reaction solution is concentrated at low temperature; the residue is purified by pre-HPLC (separation method 4) preparation to obtain 4-(5-(3-amino-8-azabicyclo[3.2.1]octane-8-carbonyl)-2-bromothiophen-3-yl)-2-fluorobenzonitril e with a yield of 75.7%.

ESI-MS m/z: 434.0[M+H]⁺.

### Step e): Preparation of 4-(5-(3-amino-8-azabicyclo[3.2.1]octane-8-carbonyl)-2-(5-fluoro-3-methylbenzo[d]isoxazol-6-yl) thiophen-3-yl)-2-fluorobenzonitrile formate

4-(5-(3-amino-8-azabicyclo[3.2.1]octane-8-carbonyl)-2-bromothiophen-3-yl)-2-fluorobenzonitril e (61 mg, 0.14 mmol), 5-fluoro-3-methyl-6-(4,4,5,5-tetramethyl-1,3,2-dioxaborolane-2-yl)benzo[d]isoxazole (59.8 mg, 0.22 mmol), Na₂CO₃ (29.9 mg, 0.29 mmol), Pd(dppf)Cl₂ (20.9 mg, 0.03 mmol) are dissolved in 5 mL of dioxane and added with 1 mL of water. The mixed solution is purged by nitrogen blowing and protected with nitrogen, and reacted at 100° C. for 30 min in a microwave reactor; when TLC and LC-MS show that the reaction is completed, the reaction solution is added with water (20 mL), and extracted with ethyl acetate (10 mL×3); organic phase is then washed with saline (10 mL), dried with anhydrous sodium sulfate, filtered, and concentrated; the residue is purified by silica gel chromatography (eluent: methanol/dichloromethane=1:10); the purified product is detected for purity by LC-MS, then purified by pre-HPLC preparation (separation method 3), and freeze-dried to obtain 4-(5-(3-amino-8-azabicyclo[3.2.1]octane-8-carbonyl)-2-(5-fluoro-3-methylbenzo[d]isoxazol-6-yl) thiophen-3-yl)-2-fluorobenzonitrile formate with a yield of 21.4%.

¹H NMR (400 MHz, Methanol-d₄) δ ppm 8.55 (s, 1H), 7.87-7.66 (m, 2H), 7.61 (dt, J=23.6, 8.2 Hz, 2H),7.35 (d, J=9.8 Hz, 1H), 7.21 (d, J=8.2 Hz, 1H), 4.86 (s, 2H), 3.59 (dp, J=11.8, 5.8 Hz, 1H), 2.55 (d, J=17.6 Hz, 3H), 2.32-1.69 (m, 8H).

ESI-MS m/z: 505.1[M+H]⁺.

### Example 10

### Preparation of (R)-4-(8-(3-aminopiperidin-1-yl)-3-(p-tolyl)indolizin-2-yl)benzonitrile hydrochloride

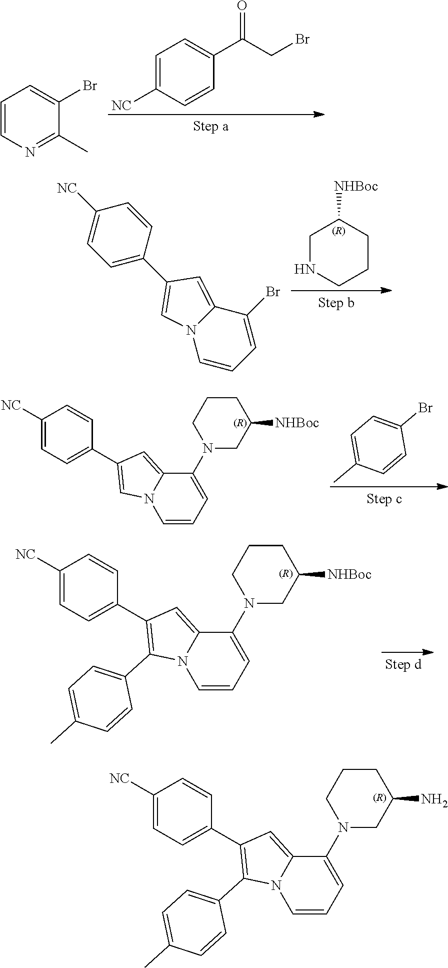

### Step a): Preparation of 4-(8-bromoindolizin-2-yl)benzonitrile

3-bromo-2-methylpyridine (500 mg, 2.94 mmol), and 4-(2-bromoacetyl)benzonitrile (791 mg, 3.53 mmol) are dissolved in toluene (12 mL), subjected to nitrogen displacement, and refluxed overnight to form a solid. The solid is then added with 10 mL of potassium carbonate aqueous solution (1.35 g, 9.78 mmol) and continued to be stirred at 80° C. for 3 h. When TLC and LC-MS show that the reaction is completed, the reaction solution is concentrated under vacuum, filtered and washed to obtain a brown solid; and the brown solid is purified by silica gel chromatography (eluent: petroleum ether/ethyl acetate=2: 1) to obtain 4-(8-bromoindolizin-2-yl)benzonitrile with a yield of 21.4%.

ESI-MS m/z: 297.9[M+H]⁺.

### Step b): Preparation of tert-butyl (R)-(1-(2-(4-cyanophenyl)bromoindolizin-8-yl)piperidin-3-yl-carbamate

4-(8-bromo-bromoindolizin-2-yl)benzonitrile (160 mg, 0.54 mmol), tert-butyl (R)-piperidin-3-carbamate (129.8 mg, 0.65 mmol), PD₂(dba)₃ (99.0 mg, 0.11 mmol), Cs₂CO₃ (325.5 mg, 1.08 mmol), and X-phos (125.2 mg, 0.22 mmol) are dissolved in toluene (12 mL), and reacted at 110° C. for 22 h under nitrogen protection. The reaction progress is monitored by TLC and LC-MS. When raw materials disappear, that is, the reaction is completed, the reaction solution is concentrated; and the residue is purified by silica gel chromatography (eluent: petroleum ether/ethyl acetate=3: 1) to obtain tert-butyl (R)-(1-(2-(4-cyanophenyl)bromoindolizin-8-yl)piperidin-3-yl-carbamate with a yield of 79.6%.

¹H NMR (400 MHz, Chloroform-d) δ ppm 7.78 (d, J=8.2 Hz, 2H), 7.69-7.54 (m, 4H), 6.83 (s, 1H), 6.45 (t, J=7.0 Hz, 1H), 6.13 (d, J=7.2 Hz, 1H), 5.16 (s, 1H), 4.02 (s, 1H), 3.40 (d, J=11.8 Hz, 1H), 3.20 (s, 1H), 3.07 (s, 2H), 1.99-1.84 (m, 2H), 1.64 (s, 2H), 1.48 (s, 9H).

ESI-MS m/z: 417.2[M+H]⁺.

### Step c): Preparation of tert-butyl (R) (1-(2-(4-cyanophenyl)-3-(p-tolyl)indolizidin-8-yl)piperidin-3-yl-carbamate

Tert-butyl (R)-(1-(2-(4-cyanophenyl)bromoindolizin-8-yl)piperidin-3-yl-carbamate (75 mg, 0.18 mmol), p-bromotoluene (37 mg, 0.22 mmol), and potassium acetate (35 mg, 0.36 mmol) are dissolved in NMP (2 mL), warmed to 100° C. under nitrogen protection, reacted for 1 h, and added with one droplet of water. The reflux reaction is then continued overnight and protected under nitrogen. The reaction progress is monitored by TLC and LC-MS; when the reaction is completed, the reaction solution is cooled and concentrated under vacuum; the concentrate is then dissolved with ethyl acetate, washed in saline (5 mL×2), dried with anhydrous sodium sulfate, filtered, and concentrated; and the residue is purified by silica gel chromatography (eluent: petroleum ether/ethyl acetate=3: 1) to obtain tert-butyl (R)-(1-(2-(4-cyanophenyl)-3-(p-tolyl)indolizidin-8-yl)piperidin-3-yl-carbamate with a yield of 44.4%.

¹H NMR (400 MHz, Chloroform-d) δ ppm 7.60-7.39 (m, 7H), 7.28 (s, 1H), 7.22 (d, J=8.0 Hz, 2H), 6.80 (s, 1H), 6.39 (t, J=7.0 Hz, 1H), 6.16 (d, J=7.2 Hz, 1H), 4.03 (s, 1H), 3.43-3.02 (m, 4H), 2.43 (s, 3H), 1.86 (t, J=12.2 Hz, 4H), 1.46 (s, 9H).

ESI-MS m/z: 507.3[M+H]⁺.

### Step d): Preparation of (R)-4-(8-(3-aminopiperidin-1-yl)-3-(p-tolyl)bromoindolizin-2-yl)benzonitrile formate

Tert-butyl (R)(1-(2-(4-cyanophenyl)-3-(p-tolyl)indolizidin-8-yl)piperidin-3-yl-carbamate (43 mg, 0.08 mmol) is dissolved in a 1M HCl (ethyl acetate solution, 2.4 mL) and reacted for 30 min at room temperature under nitrogen protection. The reaction progress is monitored by TLC and LC-MS; when the reaction is completed, the reaction solution is concentrated under low temperature to obtain a crude product; and the crude product is purified by pre-HPLC (separation method 3) and freeze-dried to obtain (R)-4-(8-(3-aminopiperidin-1-yl)-3-(p-tolyl)bromoindolizin-2-yl) benzonitrile formate with a yield of 50.0%.

¹H NMR (400 MHz, Methanol-d₄) δ ppm 8.55 (s, 1H), 7.60-7.45 (m, 5H), 7.33 (d, J=7.6 Hz, 2H), 7.21 (d, J=7.8 Hz, 2H), 6.79 (s, 1H), 6.45 (t, J=7.0 Hz, 1H), 6.29 (d, J=7.0 Hz, 1H), 3.64-3.54 (m, 1H), 3.35 (d, J=10.2 Hz, 1H), 2.90 (dt, J=52.4, 10.8 Hz, 2H), 2.43 (s, 3H), 2.28-1.35 (m, 5H).

ESI-MS m/z: 407.2[M+H]⁺.

### Example 11

### Preparation of 4-(6-(4-aminopiperidin-1-yl)-3-(3-fluoro-4-methoxyphenyl)-4-hydroxypyridin-2-yl)-2-fluoroben zonitrile formate

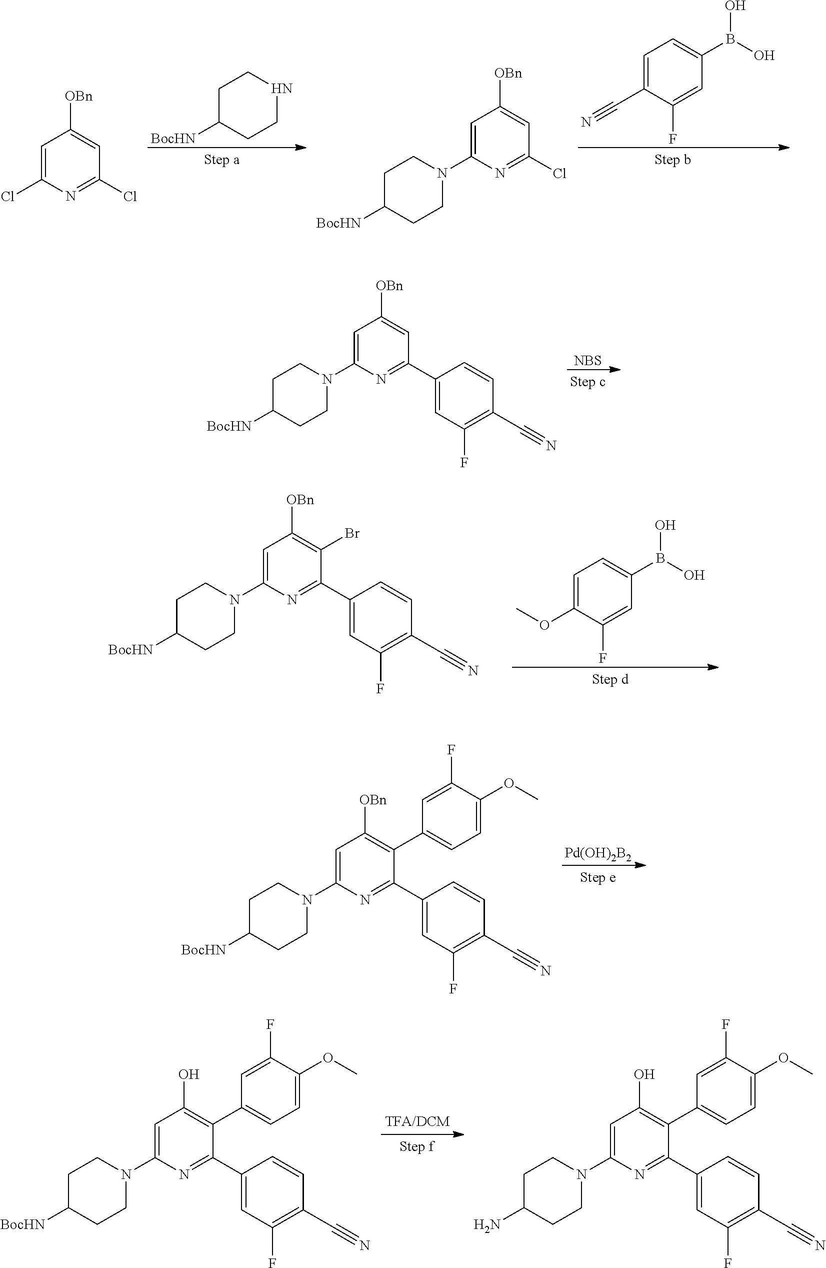

### Step a): Preparation of tert-butyl (1-(4-(benzyloxy)-6-chloropyridin-2-yl)piperidin-4-yl)carbamate

4-(benzyloxy)-2,6-dichloropyridine (1.8 g, 7.1 mmol), tert-butylpiperidin-4-carbamate (1.4 g, 7.1 mmol), and DIPEA (415 mg, 14.2 mg) are dissolved in NMP (20 mL), warmed to 130° C., reacted for 2 h. The reaction progress is monitored by LC-MS, when the reaction is completed, the reaction solution is cooled to room temperature, then added with water (50 mL) and extracted with ethyl acetate (30 mL×3). Organic layers are combined and then washed with saline (20 mL), dried with anhydrous sodium sulfate, filtered, and concentrated under vacuum; and the residue is purified by silica gel chromatography (eluent: petroleum ether/ethyl acetate=2: 1) to obtain tert-butyl (1-(4-(benzyloxy)-6-chloropyridin-2-yl)piperidin-4-yl)carbamate with a yield of 77.4%.

ESI-MS m/z=418.1 [M+H]⁺.

### Step b): Preparation of tert-butyl (1-(4-(benzyloxy)-6-(4-cyano-3-fluorophenyl)pyridin-2-yl)piperidin-4-yl)carbamate

Tert-butyl (1-(4-(benzyloxy)-6-chloropyridin-2-yl)piperidin-4-yl)carbamate (2.3 g, 5.5 mmol), (4-cyano-3-fluorophenyl)boronic acid (909 mg, 5.5 mmol), Cs₂CO₃ (3.5 g, 11.0 mmol), and Pd(dppf)Cl₂ (77.2 mg, 0.11 mmol) are dissolved in dioxane (40 mL) and added with water (4 mL), nitrogen displacement is performed for three times, and the reaction is warmed to 100° C. and performed for 2 h, the reaction process is monitored by LC-MS. When LC-MS shows that the reaction is completed, the reaction solution is cooled to room temperature, added with water (50 mL), and extracted with ethyl acetate (30 mL×3); organic layers are combined and then washed with saline (20 mL), dried with anhydrous sodium sulfate, filtered, and concentrated under vacuum; and the residue is purified by silica gel chromatography (eluent: petroleum ether/ethyl acetate=1/1) to obtain tert-butyl (1-(4-(benzyloxy)-6-(4-cyano-3-fluorophenyl)pyridin-2-yl)piperidin-4-yl)carbamate with a yield of 76.0%.

¹H NMR (400 MHz, Chloroform-d) δppm 7.83 (dd, J=17.8, 9.4 Hz, 2H), 7.65 (t, J=6.8 Hz, 1H), 7.46-7.35 (m, 5H), 6.26 (s, 1H), 5.14 (d, J=2.6 Hz, 2H), 4.28 (d, J=13.2 Hz, 2H), 3.03 (t, J=12.6 Hz, 2H), 2.06 (d, J=12.2 Hz, 2H), 1.46 (d, J=2.6 Hz, 9H).

ESI-MS m/z=503.1 [M+H]⁺.

### Step c): Preparation of tert-butyl (1-(4-(benzyloxy)-5-bromo-6-(4-cyano-3-fluorophenyl)pyridin-2-yl)piperidin-4-yl)carbamate

Tert-butyl (1-(4-(benzyloxy)-6-(4-cyano-3-fluorophenyl)pyridin-2-yl)piperidin-4-yl)carbamate (1.8 g, 3.58 mmol), and NBS (687 mg, 2.41 mmol) are dissolved in DMF (30 mL), and reacted for 2 h at room temperature, the reaction process is monitored by LC-MS. When LC-MS shows that the reaction is completed, the reaction solution is added with water (80 mL), and extracted with ethyl acetate (40 mL×3); organic layers are combined and then washed with saline (20 mL), dried with anhydrous sodium sulfate, filtered, and concentrated under vacuum; and the residue is purified by silica gel chromatography (eluent: petroleum ether/ethyl acetate=1: 2) to obtain tert-butyl (1-(4-(benzyloxy)-5-bromo-6-(4-cyano-3-fluorophenyl)pyridin-2-yl)piperidin-4-yl)carbamate with a yield of 81.0%.

ESI-MS m/z=582.1 [M+H]⁺.

### Step d): Preparation of tert-butyl (1-(4-(benzyloxy)-6-(4-cyano-3-fluorophenyl)-5-(3-fluoro-4-methoxyphenyl)pyridin-2-yl)piperi din-4-yl)carbamate

### Tert-butyl

(1-(4-(benzyloxy)-5-bromo-6-(4-cyano-3-fluorophenyl)pyridin-2-yl)piperidin-4-yl)carbamate (1.7 g, 2.9 mmol), (3-fluoro-4-methoxyphenyl)boronic acid (498 mg, 2.9 mmol), Cs₂CO₃ (1.9 g, 5.8 mmol), and Pd(dppf)Cl₂ (214 mg, 0.29 mmol) are dissolved in dioxane (30 mL) and added with water (6 mL), nitrogen displacement is performed for three times, and the reaction is warmed to 100° C. and performed for 2 h, the reaction process is monitored by LC-MS. When LC-MS shows that the raw materials disappear, the reaction is completed. The reaction solution is cooled to room temperature, then added with water (40 mL) and extracted with ethyl acetate (40 mL×3). Organic layers are combined and then washed with saturated saline (20 mL, dried with anhydrous sodium sulfate, filtered, and concentrated under vacuum; and the residue is purified by silica gel chromatography (eluent: petroleum ether/ethyl acetate=1: 1) to obtain tert-butyl (1-(4-(benzyloxy)-6-(4-cyano-3-fluorophenyl)-5-(3-fluoro-4-methoxyphenyl)pyridin-2-yl)piperi din-4-yl)carbamate with a yield of 43.8%.

ESI-MS m/z=627.1 [M+H]⁺.

### Step e): Preparation of tert-butyl (1-(6-(4-cyano-3-fluorophenyl)-5-(3-fluoro-4-methoxyphenyl)-4-hydroxypyridin-2-yl)piperidin-4-yl-carbamate

### Tert-butyl

(1-(4-(benzyloxy)-6-(4-cyano-3-fluorophenyl)-5-(3-fluoro-4-methoxyphenyl)pyridin-2-yl)piperi din-4-yl) carbamate (200 mg, 0.32 mmol) is dissolved in methanol (20 mL) and added with 100 mg of 10% Pd(OH)₂, nitrogen displacement is performed for three times, and the reaction is performed for 4 h at room temperature, the reaction process is monitored by LC-MS. When LC-MS shows that the reaction is completed, the reaction solution is sonicated for 15 min and then filtered, and the filtrate is concentrated under vacuum to obtain tert-butyl (1-(6-(4-cyano-3-fluorophenyl)-5-(3-fluoro-4-methoxyphenyl)-4-hydroxypyridin-2-yl)piperidin-4-yl-carbamate with a yield of 96.8%.

ESI-MS m/z=537.1 [M+H]⁺.

### Step f): Preparation of 4-(6-(4-aminopiperidin-1-yl)-3-(3-fluoro-4-methoxyphenyl)-4-hydroxypyridin-2-yl)-2-fluoroben zonitrile formate

### Tert-butyl

(1-(6-(4-cyano-3-fluorophenyl)-5-(3-fluoro-4-methoxyphenyl)-4-hydroxypyridin-2-yl)piperidin-4-yl-carbamate (170 mg, 0.31 mmol) is dissolved in 4 mL of TFA/DCM (1:3) solution and reacted for 1 h at room temperature. A solid is generated in the reaction solution; when LC-MS detection shows that the reaction is completed, the reaction solution is filtered to obtain a solid; and the solid is then purified by pre-HPLC preparation (separation method 3) to obtain 4-(6-(4-aminopiperidin-1-yl)-3-(3-fluoro-4-methoxyphenyl)-4-hydroxypyridin-2-yl)-2-fluoroben zonitrile formate with a yield of 6.5%.

¹H NMR (400 MHz, DMSO-d₆) δ ppm 8.71 (s, 1H), 8.30 (s, 1H), 7.75 (t, J=7.6 Hz, 1H), 7.33 (d, J=10.8 Hz, 1H), 7.13 (d, J=8.2 Hz, 1H), 7.04-6.88 (m, 2H), 6.72 (d, J=8.6 Hz, 1H), 6.41 (s, 1H), 4.26 (d, J=13.2 Hz, 2H), 3.81 (s, 3H), 3.44-3.21 (m, 3H), 2.90 (t, J=12.8 Hz, 2H), 1.94 (d, J=12.2 Hz, 2H), 1.48 (qd, J=12.2, 4.0 Hz, 2H).

ESI-MS m/z=437.1 [M+H]⁺.

### Example 12

### Preparation of (2-(4-(1H-pyrazol-1-yl)phenyl)-6-(2-(((1R,2S)-2-(4-fluorophenyl)cyclopropyl)amino)ethyl)pyri midin-4-yl) (4-(methylsulfonyl)piperazin-1-yl)methanone

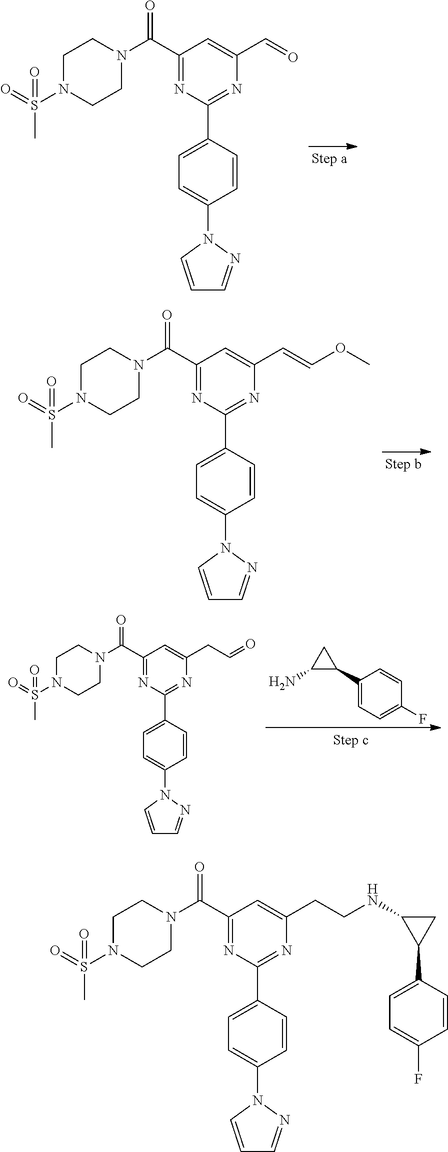

### Step a): Preparation of methyl 2-chloro-6-(4-cyano-3-fluorophenyl)pyrimidin-4-carboxylate

2-(4-(1H-pyrazol-1-yl)phenyl)-6-(4-(methylsulfonyl)piperazin-1-carbonyl)pyrimidin-4-formalde hyde (200.0 mg, 0.45 mmol),(methoxymethyl)triphenylphosphonium chloride (212.0 mg, 0.73 mmol), tris(3,6-dioxahepyl)amine (161.7 mg, 0.5 mmol) are added to a mixed solution consisting of 10 mL of dichloromethane and 10 mL of saturated potassium carbonate aqueous solution. The reaction solution is warmed to 45° C. for reflux reaction for 18 h. When LC MS shows that reaction is completed, the reaction solution is quenched with 20 mL of water, and extracted with ethyl acetate (20 mL×2); organic phase is combined, washed with saturated saline (30 mL×2), dried with anhydrous sodium sulfate, and filtered to obtain filtrate; the filtrate is concentrated under a reduced pressure; and the residue is purified by silica gel chromatography (eluent: petroleum ether/ethyl acetate=2/1) to obtain a yellow solid, (E)-(2-(4-(1H-pyrazol-1-yl)phenyl)-6-(2-methoxyvinyl)pyrimidin-4-yl) (4-(methylsulfonyl)piperazin-1-yl)methanone with a yield of 46.7%.

ESI-MS m/z: 469.5 [M+H]⁺.

### Step b): Preparation of 2-(2-(4-(1H-pyrazol-1-yl)phenyl)-6-(4-(methylsulfonyl)piperazin-1-carbonyl)pyrimidin-4-yl)ace taldehyde

(E)-(2-(4-(1H-pyrazol-1-yl)phenyl)-6-(2-methoxyvinyl)pyrimidin-4-yl)(4-(methylsulfonyl)piper azin-1-yl)methanone (95.5 mg, 0.21 mmol) is dissolved in 4 mL of 1,4-dioxane, and added dropwise with 4 mL of 6M hydrochloric acid aqueous solution. When dropwise addition is completed, the mixture is warmed to room temperature and reacted for 3 h. When LC-MS monitoring shows that the reaction is completed, the reaction solution is added to a saturated sodium bicarbonate aqueous solution (20 mL), and extracted with ethyl acetate (20 mL×2); organic phase is combined, washed with saturated saline (30 mL×2), dried with anhydrous sodium sulfate, and filtered to obtain filtrate; and the filtrate is concentrated under a reduced pressure. A crude product 2-(2-(4-(1H-pyrazol-1-yl)phenyl)-6-(4-(methylsulfonyl)piperazin-1-carbonyl)pyrimidin-4-yl)ace taldehyde is obtained.

ESI-MS m/z: 455.1 [M+H]⁺.

### Step c): Preparation of (2-(4-(1H-pyrazol-1-yl)phenyl)-6-(2-(((1R,2S)-2-(4-fluorophenyl)cyclopropyl)amino)ethyl)pyrimidin-4-yl) (4-(methylsulfonyl)piperazin-1-yl)methanone

The crude product 2-(2-(4-(1H-pyrazol-1-yl)phenyl)-6-(4-(methylsulfonyl)piperazin-1-carbonyl)pyrimidin-4-yl)ace taldehyde (41.2 mg, 0.22 mmol) obtained in Step b), 0.5 mL of methanol, and 0.1 mL of acetic acid are added to 2 mL of DCE. The reaction solution is stirred for 1 h at room temperature, then cooled to 5° C., added with sodium cyanoborohydride (55.4 mg, 0.88 mmol) in batches. The reaction solution is warmed to room temperature and reacted for 1 h; when LC-MS monitors and shows that the reaction is completed, the reaction solution is added with water (10 mL), and extracted with ethyl acetate (10 mL×3). Organic layers are combined, washed with a saturated sodium chloride aqueous solution (30 mL×2), dried with anhydrous sodium sulfate, then concentrated to dryness, and purified by Prep-HPLC (separation method 4) to obtain (2-(4-(1H-pyrazol-1-yl)phenyl)-6-(2-(((1R,2S)-2-(4-fluorophenyl)cyclopropyl)amino)ethyl)pyri midin-4-yl) (4-(methylsulfonyl)piperazin-1-yl)methanone, with a total yield of two steps being 4.1%.

¹H NMR (400 MHz, DMSO-d₆) δ ppm: 8.42 (dd, J=8.7, 1.8 Hz, 2H), 7.96 (s, 1H), 7.83-7.63 (m, 3H), 7.42 (s, 1H), 6.96 (dd, J=8.2, 5.4 Hz, 2H), 6.87 (td, J=8.6, 1.8 Hz, 2H), 6.46 (q, J=2.0 Hz, 1H), 3.79 (dd, J=40.8, 5.2 Hz, 4H), 3.34 (dt, J=18.9, 6.2 Hz, 5H), 3.17 (d, J=6.6 Hz, 2H), 2.80 (d, J=1.6 Hz, 3H), 2.43 (d, J=5.8 Hz, 1H), 1.19 (s, 2H), 0.98 (t, J=6.2 Hz, 1H), 0.79 (s, 1H).

ESI-MS m/z: 590.7 [M+H]⁺.

### Example 13

### Preparation of 2-fluoro-4-(2-(6-fluoro-1-methyl-1H-indol-5-yl)-6-(4-methylpiperazin-1-carbonyl)pyrimidin-4-y 1)benzonitrile

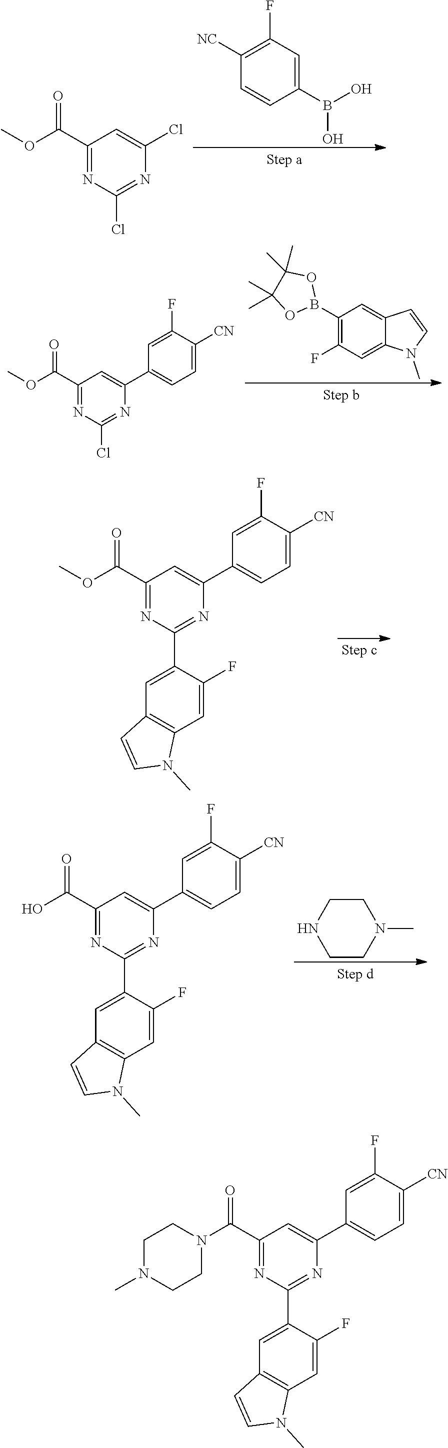

### Step a): Preparation of methyl 2-chloro-6-(4-cyano-3-fluorophenyl)pyrimidine-4-carboxylate

Raw materials, i.e., methyl 2,6-dichloropyrimidine-4-carboxylate (1 g, 4.83 mmol), pyrimidine-4-carboxylic acid (796 mg, 4.83 mmol), K₂CO₃ (1.99 g, 14.49 mmol), and Pd(dppf)Cl₂ (70.6 mg, 0.966 mmol) are dissolved in 15 mL of dioxane, and added with 3 mL of water, nitrogen displacement is performed for three times, and the reaction is warmed to 80° C. and performed for 2 h, the reaction process is monitored by LC-MS. When LC-MS shows that the raw materials are reacted completely, the reaction solution is cooled to room temperature, then added with water (40 mL) and extracted with ethyl acetate (40 mL×3). Organic layers are combined, washed with saline (40 mL×2), dried with anhydrous sodium sulfate, filtered, and concentrated under vacuum to obtain a crude product. The residue is purified by silica gel chromatography (eluent: petroleum ether/ethyl acetate=2/1) to obtain a white solid compound, methyl 2-chloro-6-(4-cyano-3-fluorophenyl)pyrimidine-4-carboxylate with a yield of 19.9%.

ESI-MS m/z=292.1 [M+H]⁺.

### Step b): Preparation of methyl 6-(4-cyano-3-fluorophenyl)-2-(6-fluoro-1-methyl-1H-indol-5-yl)pyrimidine-4-carboxylate

Methyl 2-chloro-6-(4-cyano-3-fluorophenyl)pyrimidine-4-carboxylate (140 mg, 0.50 mmol), 6-fluoro-1-methyl-5-(4,4,5,5-tetramethyl-1,3,2-dioxolane-2-yl)-1H-indole (275 mg, 1.0 mmol), Cs₂CO₃ (325 mg, 1.0 mmol), and Pd(dppf)Cl₂ (70.1 mg, 0.1 mmol) are dissolved in 5 mL of dioxane, and added with 1 mL of water, nitrogen displacement is performed for three times, and the reaction is warmed to 100° C. and reacted for 2 h, the reaction process is monitored by LC-MS. When LC-MS shows that the reaction is completed, the reaction solution is cooled to room temperature, then added with water (20 mL) and extracted with ethyl acetate (20 mL×3). Organic layers are combined, washed with saturated saline (40 mL), dried with anhydrous sodium sulfate, filtered, and concentrated under vacuum to obtain a crude product. The residue is purified by silica gel chromatography (eluent: petroleum ether/ethyl acetate=1/1) to obtain methyl 6-(4-cyano-3-fluorophenyl)-2-(6-fluoro-1-methyl-1H-indol-5-yl)pyrimidine-4-carboxylate with a yield of 64.0%.

ESI-MS m/z=405.1 [M+H]⁺.

### Step c): Preparation of 6-(4-cyano-3-fluorophenyl)-2-(6-fluoro-1-methyl-1H-indol-5-yl)pyrimidine-4-carboxylic acid

### Methyl

6-(4-cyano-3-fluorophenyl)-2-(6-fluoro-1-methyl-1H-indol-5-yl)pyrimidine-4-carboxylate (140 mg, 0.32 mmol) and lithium hydroxide (53.6 mg, 1.28 mmol) are dissolved in 5 mL of THF and 1 mL of H₂O, and reacted for 1 h at room temperature, the reaction process is monitored by LC-MS. When LC-MS shows that the reaction is completed, the reaction solution is adjusted to pH=5 by dropwise adding 1N hydrochloric acid aqueous solution, and extracted with ethyl acetate (20 mL×3). Organic layers are combined and then washed with saline (20 mL), dried with anhydrous sodium sulfate, filtered, and concentrated under vacuum to obtain a white solid compound, i.e., 6-(4-cyano-3-fluorophenyl)-2-(6-fluoro-1-methyl-1H-indol-5-yl)pyrimidine-4-carboxylic acid with a yield of 96.9%.

ESI-MS m/z=391.1 [M+H]⁺.

### Step d): Preparation of 2-fluoro-4-(2-(6-fluoro-1-methyl-1H-indol-5-yl)-6-(4-methylpiperazin-1-carbonyl)pyrimidin-4-y 1)benzonitrile

A compound methyl 6-(4-cyano-3-fluorophenyl)-2-(6-fluoro-1-methyl-1H-indol-5-yl)pyrimidine-4-carboxylic acid (130 mg, 0.33 mmol), 1-methylpiperazine (19.2 mg, 0.192 mmol), HATU (53.6 mg, 0.141 mmol) and DIPEA (49.6 mg, 0.385 mmol) are dissolved in 5 mL of DMF, and reacted for 2 h at room temperature, the reaction process is monitored by LC-MS. When LC-MS shows that the reaction is completed, the reaction solution is added with water (20 mL) and extracted with ethyl acetate (10 mL×3). Organic layers are combined and then washed with saline (20 mL), dried with anhydrous sodium sulfate, filtered, and concentrated under vacuum to obtain a crude product; and the crude product is purified by Prep-HPLC (separation method 4) to obtain a white sold compound, 2-fluoro-4-(2-(6-fluoro-1-methyl-1H-indol-5-yl)-6-(4-methylpiperazin-1-carbonyl)pyrimidin-4-y 1)benzonitrile with a yield of 54.5%.

¹H NMR (400 MHz, Methanol-d₄) δ ppm 8.44-8.39 (m, 1H), 8.33 (t, J=10.8 Hz, 2H), 8.11 (d, J=1.7 Hz, 1H), 7.96 (s, 1H), 7.31-7.21 (m, 2H), 6.62-6.55 (m, 1H), 3.87 (d, J=5.4 Hz, 2H), 3.83 (d, J=1.6 Hz, 3H), 3.73 (t, J=5.0 Hz, 2H), 2.62 (dt, J=18.2, 5.0 Hz, 4H), 2.39 (s, 3H).

ESI-MS m/z=473.1 [M+H]⁺.

Compounds in Examples 14-63 are prepared according to the synthesis method of Example 11 (separation method for the compounds: free alkali, hydrochloride and formate are separately prepared according to separation methods 4, 1 and 3, respectively), and their structures and characterization data are as follows:

| Ex- | | | | MS |
|---|---|---|---|---|
| amples | Chemical name | Structure | ¹H NMR | (M + H)⁺ |
| 14 | 4-(6-(3-amino-8-a- zabicyclo[3.2.1]oc- tane-8-yl)-3-(3- fluoro-4-methoxy- phenyl)-4-hydroxy- pyridin-2-yl)-2- fluorobenzonitrile | 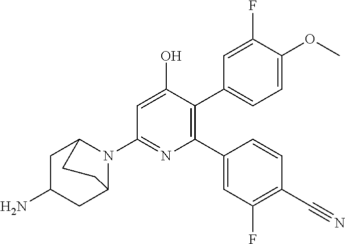 [SMILES: COc1ccc(-c2c(O)cc(N3C4CCC3CC(N)C4)nc2-c2ccc(C#N)c(F)c2)cc1F] | ¹H NMR (400 MHz, DMSO-d₆) δ ppm 7.73 (t, J = 7.4 Hz, 1H), 7.29 (d, J = 10.8 Hz, 1H), 7.14 (d, J = 8.0 Hz, 1H), 7.06-6.88 (m, 2H), 6.72 (d, J = 8.6 Hz, 1H), 6.17 (s, 1H), 4.39 (d, J = 4.8 Hz, 2H), 3.81 (s, 3H), 3.18-3.11 (m, 1H), 1.93 (dd, J = 8.6, 4.2 Hz, 2H), 1.81-1.55 (m, 4H), 1.44 (t, J = 12.0 Hz, 2H). | 463.2 |
| 15 | 2-fluoro-4-(3-(3- fluoro-4-methoxy- phenyl)-4-hydroxy- 6-(4-methylpiper- azin-1-yl)pyridin- 2-yl)benzonitrile | 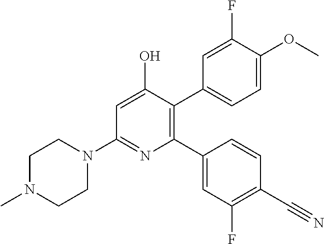 | ¹H NMR (400 MHz, DMSO-d₆) δ ppm 10.45 (s, 1H), 7.73 (t, J = 8.0 Hz, 1H), 7.31 (d, J = 8.0 Hz, 1H), 7.13 (d, J = 8.2 Hz, 1H), 7.01-6.99 (m, 2H), 6.74 (d, J = 8.2 Hz, 1H), 6.32 (s, 1H), 3.80 (s, 3H), 3.47-3.43 (m, 4H), 2.45-2.39 (m, 4H), 2.24 (s, 3H). | 437.2 |
| 16 | 2-fluoro-4-(3-(3- fluoro-4-methoxy- phenyl)-4-hydroxy- 6-(3-(methylami- no)piperidin-1-yl) pyridin-2-yl)benzo- nitrile | 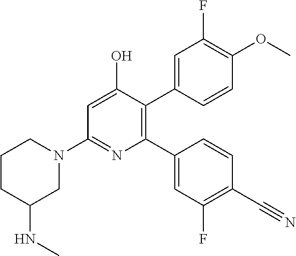 [SMILES: CNC1CCCN(c2cc(O)c(-c3ccc(OC)c(F)c3)c(-c3ccc(C#N)c(F)c3)n2)C1] | ¹H NMR (400 MHz, DMSO-d₆) δ ppm 7.73 (t, J = 7.4 Hz, 1H), 7.31 (d, J = 10.8 Hz, 1H), 7.14 (d, J = 8.0 Hz, 1H), 7.05-6.86 (m, 2H), 6.73 (d, J = 8.3 Hz, 1H), 6.32 (s, 1H), 4.15 (dd, J = 13.0, 3.6 Hz, 1H), 4.01 (dt, J = 13.6, 4.1 Hz, 1H), 3.80 (d, J = 1.2 Hz, 3H), 2.90 (d, J = 2.2 Hz, 1H), 2.66 (dd, J = 12.6, 9.3 Hz, 1H), 2.40 (tt, J = 9.0, 3.8 Hz, 1H), 2.33 (d, J = 1.2 Hz, 1H), 2.04-1.86 (m, 1H), 1.70 (dt, J = 13.4, 4.0 Hz, 1H), 1.44 (q, J = 11.8 Hz, 1H), 1.36-1.16 (m, 1H). | 451.2 |
| 17 | 4-(6-(4-aminopiper- idin-1-yl)-4-hy droxy-3-(imidazolo [1,2-a]pyridin-7-yl) pyridin-2-yl)-2- fluorobenzonitrile hydrochloride | 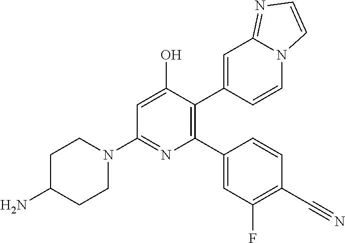 [SMILES: N#Cc1ccc(-c2nc(N3CCC(N)CC3)cc(O)c2-c2ccn3ccnc3c2)cc1F] | ¹H NMR (400 MHz, DMSO-d₆) δ ppm 14.46 (s, 1H), 11.35 (s, 1H), 8.70 (d, J = 7.0 Hz, 1H), 8.26 (s, 1H), 8.11 (d, J = 2.2 Hz, 1H), 7.82-7.62 (m, 2H), 7.51 (d, J = 10.3 Hz, 1H), 7.17 (dd, J = 26.7, 7.6 Hz, 2H), 6.49 (s, 1H), 4.34 (d, J = 13.2 Hz, 2H), 3.33 (td, J = 11.2, 5.4 Hz, 1H), 2.98 (t, J = 12.8 Hz, 2H), 2.01 (dd, J = 12.8, 3.8 Hz, 2H), 1.53 (qd, J = 12.2, 4.0 Hz, 2H). | 429.2 |
| 18 | 4-(6-(4-aminopiper- idin-1-y)-3-(3- fluoro-4-(trifluoro- methoxy)phenyl)- 4-hydroxypyridin- 2-yl)-2-fluoro- benzonitrile | 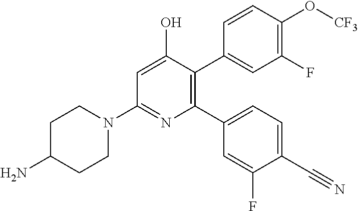 [SMILES: N#Cc1ccc(-c2nc(N3CCC(N)CC3)cc(O)c2-c2ccc(OC(F)(F)F)c(F)c2)cc1F] | ¹H NMR (400 MHz, DMSO-d₆) δ ppm 7.75 (t, J = 7.4 Hz, 1H), 7.38 (t, J = 8.4 Hz, 1H), 7.27 (dd, J = 11.2, 6.6 Hz, 2H), 7.14 (d, J = 8.2 Hz, 1H), 6.90 (d, J = 8.5 Hz, 1H), 6.30 (s, 1H), 4.14 (d, J = 13.2 Hz, 2H), 3.43 (t, J = 6.8 Hz, 1H), 2.89 (t, J = 12.2 Hz, 2H), 1.78 (d, J = 12.2 Hz, 2H), 1.23 (t, J = 11.5 Hz, 2H). | 491.1 |
| 19 | 4-(6-(4-aminopi- peridin-1-yl)-4- hydroxy- 3-(2-oxo-2,3- dihydrobenzo[d] oxazol-5-yl)pyridin- 2-yl)-2-fluoroben- zonitrile hydrochloride | 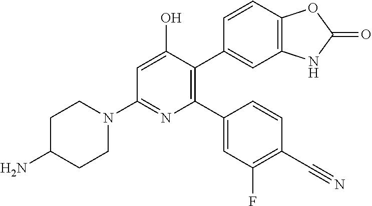 [SMILES: N#Cc1ccc(-c2nc(N3CCC(N)CC3)cc(O)c2-c2ccc3oc(=O)[nH]c3c2)cc1F] | ¹H NMR (400 MHz, DMSO-d₆) δ ppm 11.62 (s, 1H), 8.22 (s, 3H), 7.74 (t, J = 7.6 Hz, 1H), 7.52-7.30 (m, 1H), 7.14 (t, J = 7.2 Hz, 2H), 6.86 (s, 1H), 6.67 (d, J = 8.2 Hz, 1H), 6.57 (s, 1H), 4.22 (d, J = 13.4 Hz, 2H), 3.30 (s, 1H), 3.06-2.87 (m, 2H), 2.01 (d, J = 12.6 Hz, 2H), 1.58 (d, J = 12.4 Hz, 2H). | 446.2 |
| 20 | 4-(6-(4-aminopyri- din-1-yl)-3- (3-chloro- 1H-indazol-5- yl)-4-hydroxypyri- din-2-yl)-2-fluoro- benzonitrile hydrochloride | 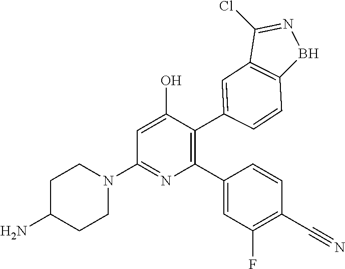 | ¹H NMR (400 MHz, DMSO-d₆) δ ppm 13.36 (s, 1H), 8.31 (s, H), 7.70 (t, J = 7.4 Hz, 1H), 7.54-7.27 (m, 4H), 7.15 (dd, J = 31.6, 8.4 Hz, 2H), 6.55 (s, 1H), 4.26 (d, J = 13.4 Hz, 2H), 3.33 (d, J = 11.4 Hz, 1H), 3.03 (t, J = 12.8 Hz, 2H), 2.03 (d, J = 12.4 Hz, 2H), 1.58 (q, J = 11.6 Hz, 2H). | 463.1 |
| 21 | 4-(6-(4-aminopiper- idin-1-yl)-4- hydroxy-3-(4-mor- pholinophenyl)pyri- din-2-yl)-2-fluoro- benzonitrile hydrochloride | 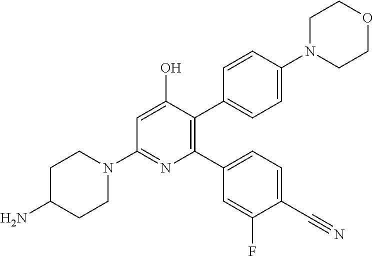 [SMILES: N#Cc1ccc(-c2nc(N3CCC(N)CC3)cc(O)c2-c2ccc(N3CCOCC3)cc2)cc1F] | ¹H NMR (400 MHz, DMSO-d₆) δ ppm 8.33 (s, 3H), 7.82 (t, J = 7.6 Hz, 1H), 7.50 (d, J = 10.4 Hz, 1H), 7.26 (d, J = 8.0 Hz, 1H), 6.94 (q, J = 8.6 Hz, 4H), 6.59 (s, 1H), 4.17 (d, J = 13.2 Hz, 2H), 3.75 (d, J = 4.8 Hz, 4H), 3.35 (td, J = 11.2, 5.2 Hz, 1H), 3.21-3.04 (m, 6H), 2.04 (d, J = 12.4 Hz, 2H), 1.61 (qd, J = 12.2, 3.8 Hz, 2H) | 474.2 |
| 22 | 4-(6-(4-aminopiper- idin-1-yl)-4-hy- droxy-6′-methoxy- [3,3′-bipyridine]-2- yl)-2-fluorobenzo- nitrile | 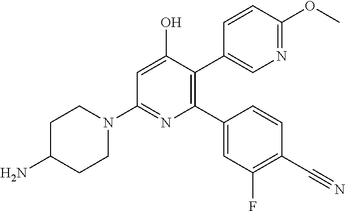 [SMILES: COc1ccc(-c2c(O)cc(N3CCC(N)CC3)nc2-c2ccc(C#N)c(F)c2)cn1] | ¹H NMR (400 MHz, Methanol-d₄) δ ppm 7.70 (d, J = 2.0 Hz, 1H), 7.60- 7.43 (m, 2H), 7.26 (d, J = 10.4 Hz, 1H), 7.15 (d, J = 8.2 Hz, 1H), 6.73 (d, J = 8.6 Hz, 1H), 6.25 (d, J = 1.4 Hz, 1H), 4.29 (d, J = 13.4 Hz, 2H), 3.86 (d, J = 1.6 Hz, 3H), 3.03 (dq, J = 10.7, 5.4, 4.4 Hz, 1H), 2.91 (t, J = 12.6 Hz, 2H), 2.02-1.86 (m, 2H), 1.49 (qd, J = 11.8, 3.8 Hz, 2H). | 420.2 |
| 23 | 4-(6-(4-aminopiper- idin-1-yl)-4- hydroxy-3- (3-hydroxy- 4-methoxyphenyl) pyridin-2-yl)-2- fluorobenzonitrile | 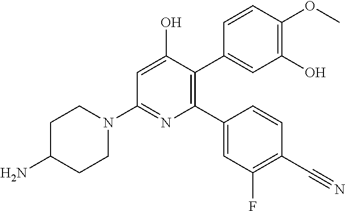 [SMILES: COc1ccc(-c2c(O)cc(N3CCC(N)CC3)nc2-c2ccc(C#N)c(F)c2)cc1O] | ¹H NMR (400 MHz, DMSO-d₆) δ ppm 8.82 (s, 1H), 7.71 (t, J = 7.4 Hz, 1H), 7.22 (dd, J = 31.8, 9.4 Hz, 2H), 6.77 (d, J = 8.2 Hz, 1H), 6.54 (s, 1H), 6.45-6.18 (m, 2H), 4.10 (d, J = 13.0 Hz, 2H), 3.73 (d, J = 1.6 Hz, 3H), 2.98-2.74 (m, 3H), 1.88-1.67 (m, 2H), 1.31-1.11 (m, 2H). | 435.2 |
| 24 | 4-(6-(3-amino-8-a- zabicyclo[3.2.1]oc- tane-8-yl)-3-(5- fluoro-3- methylben- zo[d]isoxazol-6-yl)- 4-hydroxypyridin- 2-yl)-2-fluoroben- zonitrile | 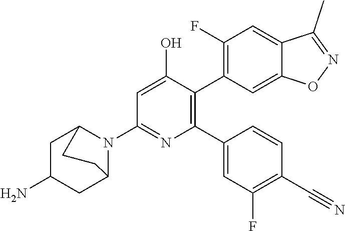 [SMILES: Cc1noc2cc(-c3c(O)cc(N4C5CCC4CC(N)C5)nc3-c3ccc(C#N)c(F)c3)c(F)cc12] | ¹H NMR (400 MHz, DMSO-d₆) δ ppm 7.69 (t, J = 7.6 Hz, 1H), 7.61 (d, J = 8.4 Hz, 1H), 7.48 (d, J = 5.4 Hz, 1H), 7.33 (d, J = 10.6 Hz, 1H), 7.11 (d, J = 8.0 Hz, 1H), 6.16 (s, 1H), 4.43 (d, J = 13.6 Hz, 2H), 2.52 (s, 3H), 1.94 (s, 2H), 1.81-1.63 (m, 4H), 1.48 (t, J = 12.0 Hz, 3H). | 488.2 |
| 25 | 4-(6-(4-aminopiper- idin-1-yl)-3-(3- fluoro-4-(3- hydroxy- 3-methyl-1-alky- nyl-1-yl)phenyl)- 4-hydroxypyridin- 2-yl)-2-fluoroben- zonitrile hydrochloride | 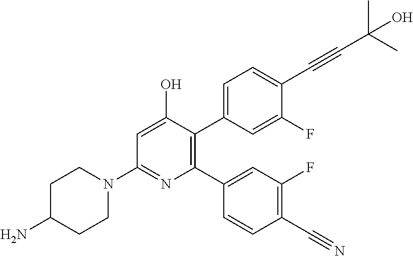 [SMILES: CC(C)(O)C#Cc1ccc(-c2c(O)cc(N3CCC(N)CC3)nc2-c2ccc(C#N)c(F)c2)cc1F] | ¹H NMR (400 MHz, Methanol-d₄) δ ppm 7.62 (dd, J = 8.0, 6.6 Hz, 1H), 7.38 (d, J = 9.8 Hz, 1H), 7.27-7.17 (m, 2H), 6.90 (dd, J = 10.2, 1.6 Hz, 1H), 6.75 (dd, J = 8.0, 1.6 Hz, 1H), 6.46 (d, J = 1.4 Hz, 1H), 4.15 (d, J = 13.4 Hz, 2H), 3.46-3.34 (m, 1H), 3.15 (s, 2H), 2.09 (d, J = 12.0 Hz, 2H), 1.77-1.58 (m, 2H), 1.46 (s, 6H). | 489.2 |
| 26 | 4-(6-(4-aminopiper- idin-1-yl)-3-(5-fluo- ro-3-methylben- zo[d]isoxazol-6- yl)-4-hydroxypyri- din-2-yl)-2-fluoro- benzonitrile | 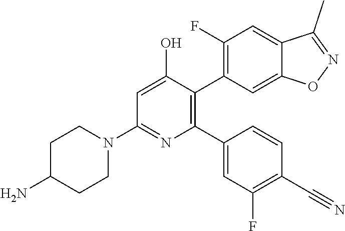 [SMILES: Cc1noc2cc(-c3c(O)cc(N4CCC(N)CC4)nc3-c3ccc(C#N)c(F)c3)c(F)cc12] | ¹H NMR (400 MHz, DMSO-d₆) δ ppm 7.70 (dd, J = 8.0, 6.8 Hz, 1H), 7.63 (d, J = 8.4 Hz, 1H), 7.52 (d, J = 5.4 Hz, 1H), 7.35 (dd, J = 10.6, 1.5 Hz, 1H), 7.11 (dd, J = 8.0, 1.6 Hz, 1H), 6.34 (s, 1H), 4.17 (d, J = 12.8 Hz, 2H), 2.92 (t, J = 12.4 Hz, 3H), 2.62 (s, 3H), 1.81 (d, J = 11.2 Hz, 2H), 1.29 (q, J = 13.8, 13.2 Hz, 2H). | 462.2 |
| 27 | 4-(6-(4-aminopiper- idin-1-yl)-3-(3- fluoro-4- methoxyphe- nyl)ethynyl)-4- hydroxypyridin-2- yl)-2-fluorobenzo- nitrile | 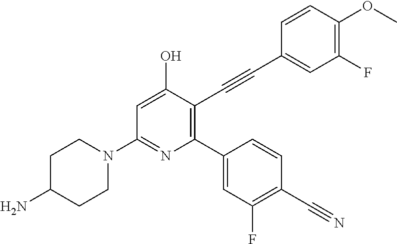 [SMILES: COc1ccc(C#Cc2c(O)cc(N3CCC(N)CC3)nc2-c2ccc(C#N)c(F)c2)cc1F] | ¹H NMR (400 MHz, DMSO-d₆) δ ppm 8.21-8.00 (m, 3H), 7.91-7.77 (m, 2H), 7.71 (dt, J = 8.6, 1.6 Hz, 1H), 7.32 (t, J = 8.8 Hz, 1H), 7.17 (d, J = 1.0 Hz, 1H), 4.31 (d, J = 13.4 Hz, 2H), 3.91 (s, 3H), 3.09-2.92 (m, 2H), 2.83 (p, J = 5.8 Hz, 1H), 1.81 (d, J = 12.3 Hz, 2H), 1.37-1.20 (m, 2H). | 461.2 |
| 28 | 4-(6-(4-aminopyri- din-1-yl)-3-(5- fluoro-1-(2- hydroxy- 2-methylpropyl)- 1H-indazol-6-yl)- 4-hydroxypyridin- 2-yl)-2-fluoroben- zonitrile hydrochloride | 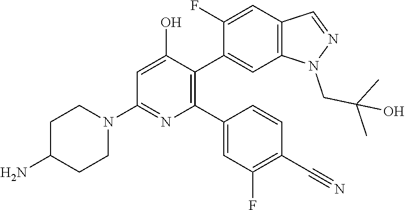 [SMILES: CC(C)(O)Cn1ncc2cc(F)c(-c3c(O)cc(N4CCC(N)CC4)nc3-c3ccc(C#N)c(F)c3)cc21] | ¹H NMR (400 MHz, Methanol-d₄) δ ppm 8.03 (s, 1H), 7.69 (dd, J = 8.0, 6.6 Hz, 1H), 7.57 (dd, J = 9.6, 1.4 Hz, 1H), 7.52-7.44 (m, 2H), 7.35 (dd, J = 8.2, 1.4 Hz, 1H), 6.67 (s, 1H), 4.27 (dd, J = 18.8, 4.4 Hz, 4H), 3.63-3.52 (m, 1H), 3.42 (dt, J = 12.8, 2.8 Hz, 2H), 2.25 (dd, J = 12.8, 3.8 Hz, 2H), 1.96-1.77 (m, 2H), 1.10 (d, J = 12.2 Hz, 6H). | 519.2 |
| 29 | 4-(6-(4-aminopiper- idin-1-yl)-4-hy- droxy-3-(3- hydroxy- 4-(trifluorometh- oxy)phenyl)pyridin- 2-yl)-2-fluoroben- zonitrile hydrochloride | 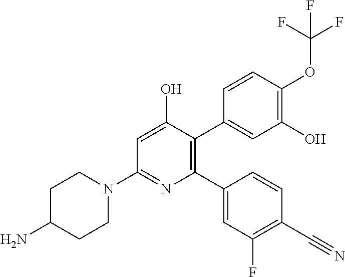 [SMILES: N#Cc1ccc(-c2nc(N3CCC(N)CC3)cc(O)c2-c2ccc(OC(F)(F)F)c(O)c2)cc1F] | ¹H NMR (400 MHz, Methanol-d₄) δ ppm 7.64 (dd, J = 8.0, 6.6 Hz, 1H), 7.40 (dd, J = 9.8, 1.4 Hz, 1H), 7.29- 7.16 (m, 1H), 7.02 (dq, J = 8.4, 1.4 Hz, 1H), 6.66 (s, 1H), 6.61-6.43 (m 2H), 4.11 (d, J = 13.6 Hz, 2H), 3.49-3.38 (m, 1H), 3.33-3.22 (m, 2H), 2.17-2.03 (m, 2H), 1.72 (qd, J = 12.4, 3.8 Hz, 2H). | 489.2 |
| 30 | 4-(6-(4-aminopiper- idin-1-yl)-3-(2,2- difluorobenzo[d] [1,3]dioxol-5-yl)- 4-hydroxypyridin- 2-yl)-2-fluoroben- zonitrile hydrochloride | 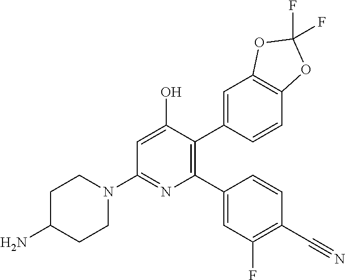 [SMILES: N#Cc1ccc(-c2nc(N3CCC(N)CC3)cc(O)c2-c2ccc3c(c2)OC(F)(F)O3)cc1F] | ¹H NMR (400 MHz, Methanol-d₄) δ ppm 7.62 (d, J = 7.6 Hz, 1H), 7.41 (d, J = 10.8 Hz, 1H), 7.19 (t, J = 8.2 Hz, 1H), 7.04-6.96 (m, 2H), 6.78-6.69 (m, 1H), 6.46 (d, J = 13.4 Hz, 1H), 4.14 (s, 2H), 3.36 (s, 1H), 3.18 (s, 2H), 2.09 (d, J = 12.4 Hz, 2H), 1.67 (d, J = 12.0 Hz, 2H). | 469.1 |
| 31 | (R)-4-(6-(3-amino- piperidin-1-yl)-3- (3-fluoro-4-methox- yphenyl)-4-hydrox- ypyridin-2-yl)- 2-fluorobenzonitrile hydrochloride | 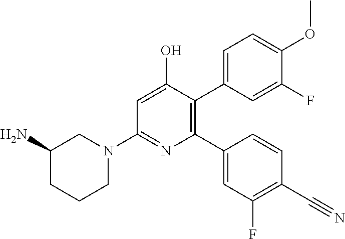 [SMILES: COc1ccc(-c2c(O)cc(N3CCC[C@@H](N)C3)nc2-c2ccc(C#N)c(F)c2)cc1F] | ¹H NMR (400 MHz, DMSO-d₆) δ ppm 8.29 (s, 3H), 7.71 (t, J = 7.4 Hz, 1H), 7.44-7.32 (m, 1H), 7.12 (dd, J = 8.2, 1.4 Hz, 1H), 7.03-6.84 (m, 2H), 6.68 (dt, J = 8.4, 1.4 Hz, 1H), 6.50 (s, 1H), 4.20 (d, J = 9.6 Hz, 1H), 3.74 (s, 4H), 3.23-2.96 (m, 3H), 2.04-1.87 (m, 1H), 1.87-1.72 (m, 1H), 1.54 (ddt, J = 19.6, 13.6, 7.0 Hz, 2H). | 437.2 |
| 32 | 4-(6-(4-aminopiper- idin-1-yl)-3-(2- fluoro-4- (2-hydroxy- 2-methylpropyl) phenyl)-4-hydrox- ypyridin-2-yl)-2- fluorobenzonitrile hydrochloride | 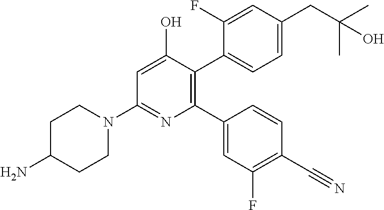 [SMILES: CC(C)(O)Cc1ccc(-c2c(O)cc(N3CCC(N)CC3)nc2-c2ccc(C#N)c(F)c2)c(F)c1] | ¹H NMR (400 MHz, Methanol-d₄) δ ppm 7.62 (t, J = 6.8 Hz, 1H), 7.37 (d, J = 9.6 Hz, 1H), 7.23 (d, J = 7.8 Hz, 1H), 7.04-6.76 (m, 3H), 6.53 (s, 1H), 4.12 (d, J = 12.6 Hz, 2H), 3.59- 3.38 (m, 3H), 2.63 (s, 2H), 2.12 (d, J = 11.8 Hz, 2H), 1.74 (d, J = 12.2 Hz, 2H), 1.04 (s, 6H). | 479.2 |
| 33 | 4-(6-(4-aminopiper- idin-1-yl)-3-(3,4- dimethoxyphenyl)- 4-hydroxypyridin- 2-yl)-2-fluoroben- zonitrile hydrochloride | 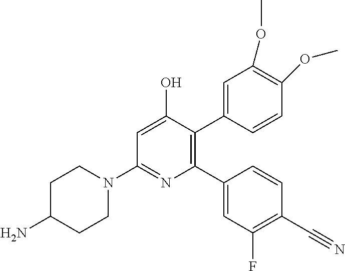 [SMILES: COc1ccc(-c2c(O)cc(N3CCC(N)CC3)nc2-c2ccc(C#N)c(F)c2)cc1OC] | ¹H NMR (400 MHz, DMSO-d₆) δ ppm 8.17 (s, 3H), 7.77 (t, J = 7.6 Hz, 1H), 7.40 (d, J = 10.4 Hz, 1H), 7.20 (d, J = 8.0 Hz, 1H), 6.83 (d, J = 8.4 Hz, 1H), 6.63 (d, J = 2.0 Hz, 1H), 6.56 (dd, J = 8.2, 2.0 Hz, 2H), 4.20 (d, J = 13.2 Hz, 2H), 3.72 (s, 3H), 3.57 (s, 3H), 3.30 (s, 1H), 2.97 (d, J = 13.4 Hz, 2H), 2.00 (d, J = 11.6 Hz, 2H), 1.65-1.50 (m, 2H). | 449.2 |
| 34 | 2-fluoro-4-(3-(3- fluoro-4-methoxy- phenyl)-4-hydroxy- 6-(4-morpholino- peridin-1-yl)pyridin- 2-yl)benzonitrile hydrochloride | 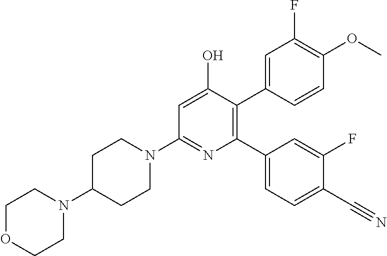 [SMILES: COc1ccc(-c2c(O)cc(N3CCC(N4CCOCC4)CC3)nc2-c2ccc(C#N)c(F)c2)cc1F] | ¹H NMR (400 MHz, DMSO-d₆) δ ppm 11.13 (m, 2H), 7.78 (dt, J = 11.8, 7.6 Hz, 1H), 7.37 (d, J = 26.0 Hz, 1H), 7.18 (t, J = 7.6 Hz, 1H), 7.00 (dtd, J = 9.8, 5.3, 4.9, 2.6 Hz, 1H), 6.80-6.67 (m, 2H), 6.48 (d, J = 49.4 Hz, 1H), 4.36 (s, 2H), 3.98 (d, J = 12.0 Hz, 2H), 3.76 (s, 5H), 3.46 (s, 3H), 3.15-3.04 (m, 2H), 2.90 (d, J = 14.4 Hz, 2H), 2.21 (s, 2H), 1.72 (s, 2H). | 507.2 |
| 35 | 4-(6-(4-aminopiper- idin-1-yl)-3-(2,3- difluoro-4- methoxyphenyl)- 4-hydroxy- pyridin-2-yl)- 2-fluorobenzonitrile | 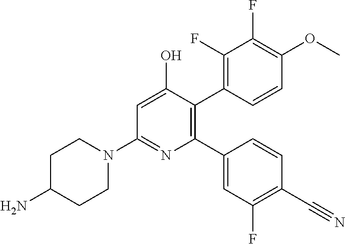 [SMILES: COc1ccc(-c2c(O)cc(N3CCC(N)CC3)nc2-c2ccc(C#N)c(F)c2)c(F)c1F] | ¹H NMR (400 MHz, DMSO-d₆) δ ppm 7.74 (dd, J = 8.2, 6.8 Hz, 1H), 7.31 (dd, J = 10.6, 1.4 Hz, 1H), 7.12 (dd, J = 8.0, 1.6 Hz, 1H), 6.89 (td, J = 8.4, 7.8, 1.6 Hz, 1H), 6.78 (td, J = 8.4, 2.0 Hz, 1H), 6.28 (s, 1H), 4.12 (d, J = 13.2 Hz, 2H), 3.85 (s, 3H), 2.90 (d, J = 11.6 Hz, 2H), 2.80 (dt, J = 10.0, 4.2 Hz, 1H), 1.76 (dd, J = 12.8, 3.8 Hz, 2H), 1.23 (q, J = 11.4 Hz, 2H). | 455.2 |
| 36 | 4-(6-(4-aminopiper- idin-1-yl)-3-(2- fluoro-4-methoxy- phenyl)-4-hydroxy- pyridin-2-yl)-2- fluorobenzonitrile hydrochloride | 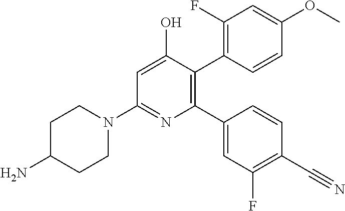 [SMILES: COc1ccc(-c2c(O)cc(N3CCC(N)CC3)nc2-c2ccc(C#N)c(F)c2)c(F)c1] | ¹H NMR (400 MHz, Methanol-d₄) δ ppm 7.74 (dd, J = 8.0, 6.6 Hz, 1H), 7.51 (dd, J = 9.8, 1.4 Hz, 1H), 7.33 (dd, J = 8.0, 1.6 Hz, 1H), 7.02 (t, J = 8.6 Hz, 1H), 6.79-6.58 (m, 3H), 4.21 (dt, J = 14.0, 3.4 Hz, 2H), 3.77 (s, 3H), 3.53 (tt, J = 11.4, 4.2 Hz, 1H), 3.44-3.30 (m, 2H), 2.34-2.14 (m, 2H), 1.98-1.76 (m, 2H). | 437.2 |
| 37 | 4-(6-(3-amino-8-a- zabicyclo[3.2.1]oc- tane-8-yl)-4- hydroxy-3- (3-hydroxy- 4-methoxyphenyl) pyridin-2-yl)-2- fluorobenzonitrile | 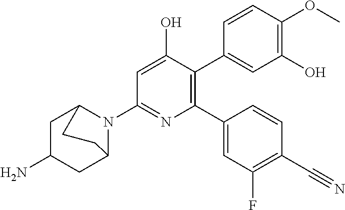 [SMILES: COc1ccc(-c2c(O)cc(N3C4CCC3CC(N)C4)nc2-c2ccc(C#N)c(F)c2)cc1O] | ¹H NMR (400 MHz, DMSO-d₆) δ ppm 8.83 (s, 1H), 7.72 (dd, J = 8.0, 7.0 Hz, 1H), 7.35-7.12 (m, 2H), 6.77 (d, J = 8.4 Hz, 1H), 6.54 (d, J = 2.0 Hz, 1H), 6.38 (dd, J = 8.2, 2.0 Hz, 1H), 6.19 (s, 1H), 4.40 (d, J = 4.2 Hz, 2H), 3.74 (s, 3H), 3.18-3.11 (m, 1H), 1.94 (dd, J = 8.6, 4.2 Hz, 2H), 1.82-1.61 (m, 4H), 1.52-1.37 (m, 2H). | 461.2 |
| 38 | 4-(6-(4-aminopiper- ine-1-yl)-3-(3- fluoro-4- (2-hydroxy- 2-methylpropoxy) phenyl)-4-hydroxy- pyridin-2-yl)- 2-fluorobenzonitrile hydrochloride | 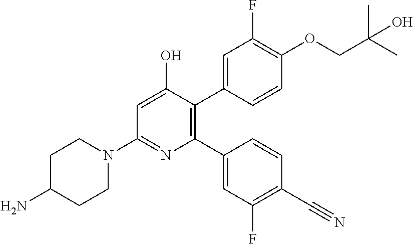 [SMILES: CC(C)(O)COc1ccc(-c2c(O)cc(N3CCC(N)CC3)nc2-c2ccc(C#N)c(F)c2)cc1F] | ¹H NMR (400 MHz, Methanol-d₄) δ ppm 7.74 (s, 1H), 7.52 (q, J = 6.8, 5.2 Hz, 1H), 7.42-7.26 (m, 1H), 6.99 (t, J = 7.0 Hz, 2H), 6.79 (d, J = 7.8 Hz, 1H), 6.63 (s, 1H), 4.30-4.14 (m, 2H), 3.81 (s, 2H), 3.53 (s, 1H), 3.36 (s, 2H), 2.27-2.14 (m, 2H), 1.85 (s, 2H), 1.30 (s, 6H). | 495.2 |
| 39 | 4-(6-(4-aminopiper- idin-1-yl)-3-(3- fluoro-4-(2- hydroxy-2- methylpropyl) phenyl)-4-hydroxy- pyridin-2-yl)-2- fluorobenzonitrile hydrochloride | 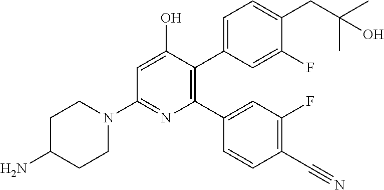 [SMILES: CC(C)(O)Cc1ccc(-c2c(O)cc(N3CCC(N)CC3)nc2-c2ccc(C#N)c(F)c2)cc1F] | ¹H NMR (400 MHz, Methanol-d₄) δ ppm 7.75 (dd, J = 8.0, 6.6 Hz, 1H), 7.51 (d, J = 1.4 Hz, 1H), 7.36 (dd, J = 8.0, 1.6 Hz, 1H), 7.24 (d, J = 7.8 Hz, 1H), 6.92 (dd, J = 10.6, 1.8 Hz, 1H), 6.84 (dd, J = 7.8, 1.7 Hz, 1H), 6.64 (s, 1H), 4.22 (dq, J = 14.2, 2.4 Hz, 2H), 3.62-3.47 (m, 1H), 3.45- 3.37 (m, 2H), 2.79 (d, J = 1.4 Hz, 2H), 2.30-2.17 (m, 2H), 1.85 (qd, J = 12.4, 4.2 Hz, 2H), 1.17 (s, 6H). | 479.2 |
| 40 | 2-fluoro-4-(3-(3- fluoro-4-methoxy- phenyl)-4-hydroxy- 6-(piperidin-4-yl- amino)pyridin-2- yl)benzonitrile | 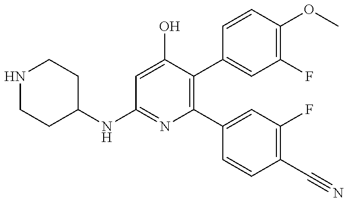 [SMILES: COc1ccc(-c2c(O)cc(NC3CCNCC3)nc2-c2ccc(C#N)c(F)c2)cc1F] | ¹H NMR (400 MHz, DMSO-d₆) δ ppm 7.72 (dd, J = 8.0, 7.0 Hz, 1H), 7.28 (dd, J = 10.8, 1.4 Hz, 1H), 7.12 (dd, J = 8.0, 1.4 Hz, 1H), 7.04-6.89 (m, 2H), 6.71 (dt, J = 8.4, 1.4 Hz, 1H), 6.46 (d, J = 7.4 Hz, 1H), 6.08 (s, 1H), 3.80 (s, 3H), 3.69 (s, 1H), 2.97 (dt, J = 12.4, 3.8 Hz, 2H), 2.63-2.53 (m, 2H), 1.87 (dd, J = 12.8, 3.8 Hz, 2H), 1.31 (qd, J = 11.6, 3.8 Hz, 2H). | 437.2 |
| 41 | 4-(6-(4-aminopiper- idin-1-yl)-3-(2,5- difluoro-4- methoxyphenyl)- 4-hydroxy- pyridin-2-yl)- 2-fluorobenzonitrile hydrochloride | 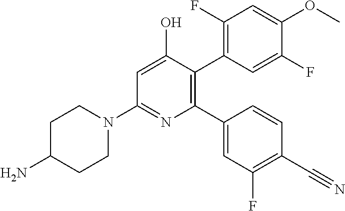 [SMILES: COc1cc(F)c(-c2c(O)cc(N3CCC(N)CC3)nc2-c2ccc(C#N)c(F)c2)cc1F] | ¹H NMR (400 MHz, DMSO-d₆) δ ppm 11.3-10.6 (s, 1H), 8.17 (dt, J = 17.6, 8.8 Hz, 2H), 7.80 (t, J = 7.6 Hz, 1H), 7.39 (d, J = 10.6 Hz, 1H), 7.18 (dd, J = 8.0, 1.6 Hz, 1H), 7.03 (ddd, J = 16.4, 11.2, 7.2 Hz, 2H), 6.48 (s, 1H), 4.25 (d, J = 13.2 Hz, 2H), 3.82 (s, 3H), 3.29 (s, 1H), 2.96 (t, J = 12.8 Hz, 2H), 2.05-1.92 (m, 2H), 1.63-1.44 (m, 2H). | 455.2 |
| 42 | 4-(6-(4-aminopiper- idin-1-yl)-3-(4- fluoro-3- hydroxyphe- nyl)-4-hydroxy- pyridin-2-yl)-2- fluorobenzonitrile | 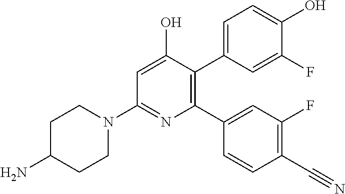 [SMILES: N#Cc1ccc(-c2nc(N3CCC(N)CC3)cc(O)c2-c2ccc(O)c(F)c2)cc1F] | ¹H NMR (400 MHz, DMSO-d₆) δ ppm 7.72 (dd, J = 8.2, 7.0 Hz, 1H), 7.25 (dd, J = 10.8, 1.4 Hz, 1H), 7.18 (dd, J = 8.2, 1.6 Hz, 1H), 6.98 (dd, J = 11.4, 8.2 Hz, 1H), 6.68 (dd, J = 8.6, 2.2 Hz, 1H), 6.41 (ddd, J = 8.4, 4.4, 2.2 Hz, 1H), 6.31 (s, 1H), 4.17 (dt, J = 13.6, 3.6 Hz, 2H), 2.98-2.79 (m, 3H), 1.88-1.75 (m, 2H), 1.28 (qd, J = 11.6, 3.8 Hz, 2H). | 423.2 |
| 43 | 6-(4-aminopiperidin- 1-yl)-3-(3-hydroxy- 4-methoxyphe- nyl)-2′-methyl-[2,4′- bipyridine]-4- ol | 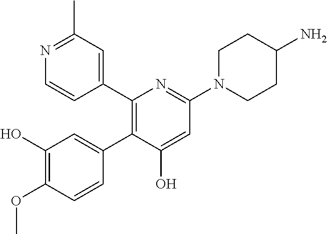 [SMILES: COc1ccc(-c2c(O)cc(N3CCC(N)CC3)nc2-c2ccnc(C)c2)cc1O] | ¹H NMR (400 MHz, DMSO-d₆) δ ppm 7.73 (t, J = 8.2 Hz, 1H), 7.31 (dd, J = 11.4, 8.2 Hz, 1H), 7.15 (dd, J = 8.4, 6.2 Hz, 1H), 7.03-6.93 (m, 2H), 6.75-6.71 (m, 1H), 6.37 (s, 1H), 4.42-4.35 (m, 1H), 4.04 (d, J = 6.8 Hz, 1H), 3.80 (s, 3H), 3.38 (s, 3H), 2.97-2.83 (m, 2H), 2.69 (m, 2H), 1.83-1.77 (m, 2H), 1.35-1.22 (m, 1H). | 407.2 |
| 44 | 4-(3-amino-4-meth- oxyphenyl)-6-(4- aminopiperidin-1- yl)-4-hydroxypyri- din-2-yl)-2-fluoro- benzonitrile | 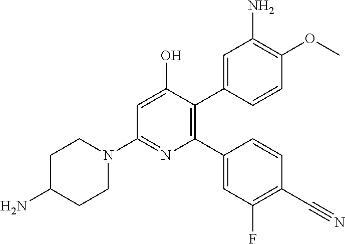 [SMILES: COc1ccc(-c2c(O)cc(N3CCC(N)CC3)nc2-c2ccc(C#N)c(F)c2)cc1N] | ¹H NMR (400 MHz, DMSO-d₆) δ ppm 7.71 (t, J = 7.6 Hz, 1H), 7.32- 7.18 (m, 2H), 6.66 (d, J = 8.2 Hz, 1H), 6.41 (d, J = 2.2 Hz, 1H), 6.32 (s, 1H), 6.15 (dd, J = 8.2, 2.2 Hz, 1H), 4.60 (d, J = 9.8 Hz, 1H), 4.12 (dt, J = 13.2, 4.0 Hz, 2H), 3.73 (s, 3H), 2.95- 2.77 (m, 2H), 1.83-1.74 (m, 2H), 1.25 (qd, J = 11.6, 4.0 Hz, 2H). | 434.2 |
| 45 | 2-fluoro-4-(3-(3- fluoro-4-methoxy- phenyl)-4-hydroxy- 6-(4-((2-hydroxy- 2-methylpropyl)a- mino)piperidin-1- yl)pyridin-2-yl)ben- zonitrile hydrochloride | 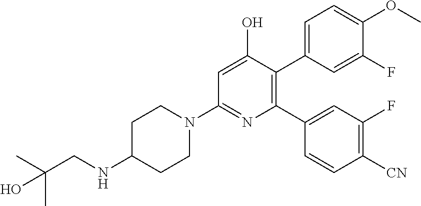 [SMILES: COc1ccc(-c2c(O)cc(N3CCC(NCC(C)(C)O)CC3)nc2-c2ccc(C#N)c(F)c2)cc1F] | ¹H NMR (400 MHz, Methanol-d₄) δ ppm 7.73-7.59 (m, 1H), 7.42 (s, 1H), 7.25 (s, 1H), 6.98-6.83 (m, 2H), 6.71 (s, 1H), 6.53 (s, 1H), 4.15 (s, 2H), 3.74 (s, 3H), 3.60-3.38 (m, 1H), 3.25 (s, 2H), 3.00 (s, 2H), 2.25 (s, 2H), 1.85 (s, 2H), 1.25 (s, 6H). | 509.2 |
| 46 | 4-(6-(4-aminopiper- idin-1-yl)-3-(3,4- difluoro-5-hydroxy- phenyl)-4-hy- droxypyridin-2-yl)- 2-fluorobenzonitrile | 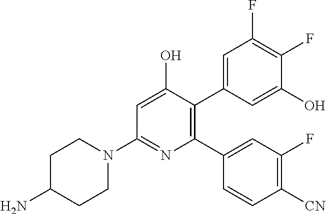 [SMILES: N#Cc1ccc(-c2nc(N3CCC(N)CC3)cc(O)c2-c2cc(O)c(F)c(F)c2)cc1F] | ¹H NMR (400 MHz, Methanol-d₄) δ ppm 7.56 (dd, J = 8.2, 6.8 Hz, 1H), 7.32 (dd, J = 10.8, 1.6 Hz, 1H), 7.22 (dd, J = 8.0, 1.6 Hz, 1H), 6.50-6.33 (m, 2H), 6.27 (s, 1H), 4.32 (d, J = 13.0 Hz, 2H), 3.17-3.01 (m, 1H), 2.95-2.81 (m, 2H), 2.05-1.88 (m, 2H), 1.52 (qd, J = 12.2, 4.2 Hz, 2H). | 441.2 |
| 47 | 4-(6-(4-aminopiper- idin-1-yl)-3-(3- cyano-4-methoxy- phenyl)-4-hydroxy- pyridin-2-yl)-2- fluorobenzonitrile | 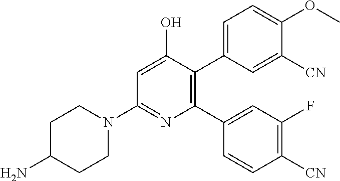 [SMILES: COc1ccc(-c2c(O)cc(N3CCC(N)CC3)nc2-c2ccc(C#N)c(F)c2)cc1C#N] | ¹H NMR (400 MHz, DMSO-d₆) δ ppm 7.73 (dd, J = 8.1, 7.0 Hz, 1H), 7.42 (d, J = 2.2 Hz, 1H), 7.33 (dd, J = 10.8, 1.4 Hz, 1H), 7.25 (dd, J = 8.8, 2.2 Hz, 1H), 7.17-7.05 (m, 2H), 6.28 (s, 1H), 4.12 (dd, J = 10.8, 6.8 Hz, 2H), 3.88 (s, 3H), 2.93-.278 (m, 3H), 1.78 (dd, J = 12.8, 4.0 Hz, 2H), 1.26 (td, J = 11.6, 7.8 Hz, 2H). | 444.2 |
| 48 | 4,4′-(6-(4-aminopi- peridin-1-yl)-4-hy- droxypyridine-2,3- diyl)bis(2-fluoro- benzonitrile) | 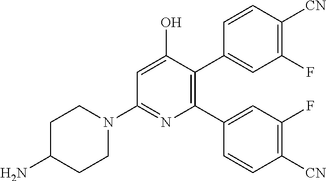 [SMILES: N#Cc1ccc(-c2nc(N3CCC(N)CC3)cc(O)c2-c2ccc(C#N)c(F)c2)cc1F] | ¹H NMR (400 MHz, DMSO-d₆) δ ppm 7.71 (dt, J = 16.0, 7.6 Hz, 2H), 7.32 (ddd, J = 16.8, 10.8, 1.6 Hz, 2H), 7.07 (dd, J = 8.0, 1.6 Hz, 1H), 6.97 (dd, J = 8.2, 1.6 Hz, 1H), 6.22 (s, 1H), 4.15 (d, J = 12.6 Hz, 2H), 2.99-2.81 (m, 3H), 1.95-1.68 (m, 2H), 1.28 (qd, J = 12.2, 11.8, 3.8 Hz, 2H). | 432.2 |
| 49 | 2-fluoro-4-(3-(3- fluoro-4-methoxy- phenyl)-4-hydroxy- 6-(4-((2-methoxy- ethyl)amino)piper- idin-1-yl)pyridin- 2-yl)benzonitrile | 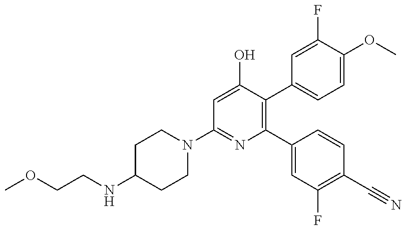 | ¹H NMR (400 MHz, DMSO-d₆) δ ppm 7.74 (t, J = 7.4 Hz, 1H), 7.41- 7.26 (m, 1H), 7.15-7.12 (m, 1H), 7.10-6.88 (m, 2H), 6.73 (dt, J = 8.4, 1.4 Hz, 1H), 6.36 (s, 1H), 4.21 (d, J = 12.8 Hz, 2H), 3.80 (s, 3H), 3.48 (d, J = 5.4 Hz, 2H), 3.32 (s, 3H), 2.90 (dd, J = 27.6, 15.2 Hz, 5H), 1.97 (d, J = 12.4 Hz, 2H), 1.39 (s, 2H). | 495.2 |
| 50 | 4-(6-amino-4-meth- ylpiperidin-1-yl)- 3-(3-fluoro-4-meth- oxyphenyl)-4-hy- droxypyridin-2- yl)-2-fluorobenzo- nitrile | 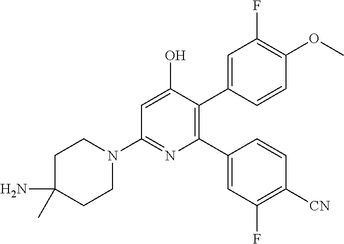 [SMILES: COc1ccc(-c2c(O)cc(N3CCC(C)(N)CC3)nc2-c2ccc(C#N)c(F)c2)cc1F] | ¹H NMR (400 MHz, DMSO-d₆ + D₂O) δ ppm 7.73 (t, J = 7.6 Hz, 1H), 7.30 (dd, J = 10.8, 1.4 Hz, 1H), 7.14 (dd, J = 8.2, 1.6 Hz, 1H), 7.04-6.91 (m, 2H), 6.72 (dd, J = 8.4, 2.2 Hz, 1H), 6.32 (s, 1H), 3.80 (s, 3H), 3.54 (q, J = 5.8 Hz, 4H), 1.47 (h, J = 8.0 Hz, 4H), 1.12 (s, 3H). | 451.2 |
| 51 | 4-(6-((3R,4R)-4-a- mino-3-methoxypi- peridin-1-yl)-3-(3- fluoro-4-methoxy- phenyl)-4- hydroxypyridin- 2-yl)-2-fluoro- benzonitrile | 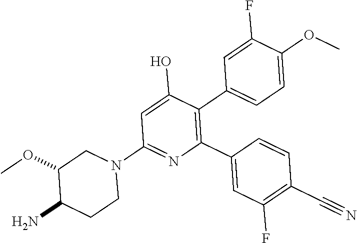 [SMILES: COc1ccc(-c2c(O)cc(N3CC[C@@H](N)[C@H](OC)C3)nc2-c2ccc(C#N)c(F)c2)cc1F] | ¹H NMR (400 MHz, DMSO-d₆) δ ppm 7.73 (t, J = 8.0 Hz, 1H), 7.31 (dd, J = 8.2, 2.4 Hz, 1H), 7.15 (dd, J = 6.4, 1.6 Hz, 1H), 7.07-6.92 (m, 2H), 6.81-6.58 (m, 1H), 6.37 (s, 1H), 4.50-4.24 (m, 1H), 4.04 (d, J = 2.4 Hz, 1H), 3.80 (s, 3H), 3.38 (s, 3H), 3.03-2.75 (m, 2H), 2.69-2.73 (m, 2H), 1.89-1.64 (m, 1H), 1.46- 1.15 (m, 1H). | 467.2 |
| 52 | 2-fluoro-4-(3-(3- fluoro-4-methoxy- phenyl)-6-(hexahy- dropyrrolo[3,4-c] pyrrole-2(1H)-yl)- 4-hydroxypyridin- 2-yl)benzonitrile hydrochloride | 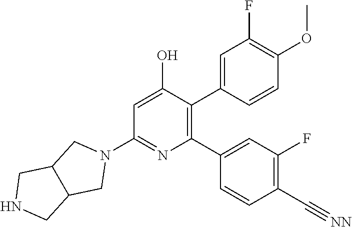 | ¹H NMR (400 MHz, Methanol-d₄) δ ppm 7.76 (dd, J = 7.8, 6.6 Hz, 1H), 7.62-7.48 (m, 1H), 7.36 (dd, J = 8.0, 1.2 Hz, 1H), 7.04-6.91 (m, 2H), 6.80 (dt, J = 8.4, 1.6 Hz, 1H), 6.38 (s, 1H), 3.93 (dd, J = 11.2, 6.2 Hz, 2H), 3.85 (s, 3H), 3.81 (dd, J = 11.2, 2.8 Hz, 2H), 3.67 (q, J = 7.8 Hz, 2H), 3.42 (d, J = 9.4 Hz, 2H), 3.33 (p, J = 1.6 Hz, 2H). | 449.2 |
| 53 | 2-fluoro-4-(3-(3- fluoro-4-methoxy- phenyl)-4-hydroxy- 6-(((tetrahydro-1H- pyrrolizin-7a(5H)- yl)methyl)ami- no)pyridin-2-yl)ben- zonitrile hydrochloride | 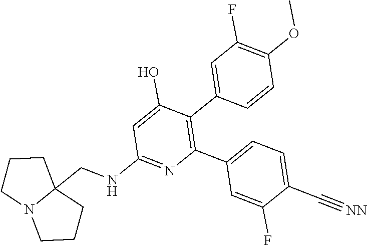 | ¹H NMR (400 MHz, DMSO-d₆) δ ppm 8.21 (s, 3H), 7.94-7.82 (m, 1H), 7.53 (d, J = 10.2 Hz, 1H), 7.26 (d, J = 8.2 Hz, 1H), 7.11-6.95 (m, 2H), 6.85-6.75 (m, 1H), 6.54 (s, 1H), 3.79 (s, 3H), 3.70 (s, 2H), 3.34 (s, 2H), 3.04 (d, J = 11.8, 2H), 2.05 (qd, J = 11.2, 10.2, 6.4 Hz, 4H), 1.92 (dd, J = 10.8, 5.4 Hz, 4H). | 477.2 |
| 54 | 4-(6-(4-aminopiper- idin-1-yl)-4-hy- droxy-3-(morpholi- nomethyl)pyridin- 2-yl)-2-fluoroben- zonitrile hydrochloride | 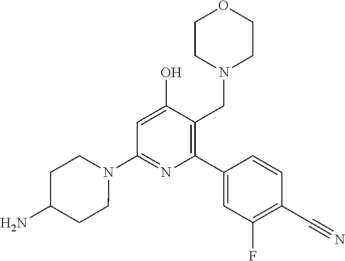 [SMILES: N#Cc1ccc(-c2nc(N3CCC(N)CC3)cc(O)c2CN2CCOCC2)cc1F] | ¹H NMR (400 MHz, Methanol-d₄) δ ppm 8.04 (s, 1H), 7.88 (d, J = 8.6 Hz, 1H), 7.81-7.63 (m, 1H), 6.66 (s, 1H), 4.23 (d, J = 9.8 Hz, 4H), 3.86 (dq, J = 21.4, 10.4 Hz, 4H), 3.54 (s, 1H), 3.38 (s, 4H), 2.99 (d, J = 11.2 Hz, 2H), 2.19 (d, J = 11.2 Hz, 2H), 1.88 (d, J = 12.0 Hz, 2H). | 412.2 |
| 55 | 2-fluoro-4-(3-(3- fluoro-4-methoxy- phenyl)-4-hydroxy- 6-(4-(methylami- no)piperidin-1-yl) pyridin-2-yl)benzo- nitrile | 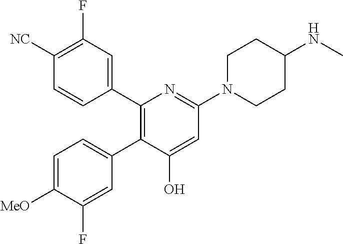 [SMILES: CNC1CCN(c2cc(O)c(-c3ccc(OC)c(F)c3)c(-c3ccc(C#N)c(F)c3)n2)CC1] | ¹H NMR (400 MHz, DMSO-d₆ + D₂O) δ ppm 7.73 (dd, J = 8.0, 6.8 Hz, 1H), 7.30 (dd, J = 10.8, 1.6 Hz, 1H), 7.14 (dd, J = 8.2, 1.6 Hz, 1H), 7.04-6.93 (m, 2H), 6.76- 6.69 (m, 1H), 6.33 (s, 1H), 4.11 (dd, J = 13.4, 4.0 Hz, 2H), 3.80 (s, 3H), 2.92 (ddd, J = 13.6, 11.2, 2.7 Hz, 2H), 2.67-2.53 (m, 1H), 2.33 (s, 3H), 1.92-1.82 (m, 2H), 1.30-1.19 (m, 2H). | 451.2 |
| 56 | 4-(6-(4-(dimethyl- amino)piperidin-1- yl)-3-(3-fluoro-4- methoxyphenyl)- 4-hydroxypyridin- 2-yl)-2-fluoroben- zonitrile | 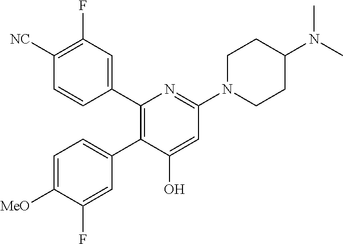 [SMILES: COc1ccc(-c2c(O)cc(N3CCC(N(C)C)CC3)nc2-c2ccc(C#N)c(F)c2)cc1F] | ¹H NMR (400 MHz, DMSO-d₆) δ ppm 10.36 (s, 1H), 7.73 (dd, J = 8.0, 6.8 Hz, 1H), 7.31 (dd, J = 10.8 ,1.6 Hz, 1H), 7.15 (dd, J = 8.2, 1.6 Hz, 1H), 7.07-6.88 (m, 2H), 6.73 (ddd, J = 8.4, 2.2, 1.2 Hz, 1H), 6.33 (s, 1H), 4.21 (dt, J = 12.8, 3.8 Hz, 2H), 3.80 (s, 3H), 2.82 (td, J = 12.8, 2.6 Hz, 2H), 2.29 (td, J = 10.6, 5.4 Hz, 1H), 2.19 (s, 6H), 1.87-1.76 (m, 2H), 1.35 (qd, J = 12.2, 4.0 Hz, 2H). | 465.2 |
| 57 | 4-(6-(4-aminopiper- idin-1-yl)-3-(3- fluoro-4-methoxy- phenyl)-4-hydroxy- pyridin-2-yl)-2- fluorobenzonitrile hydrochloride | 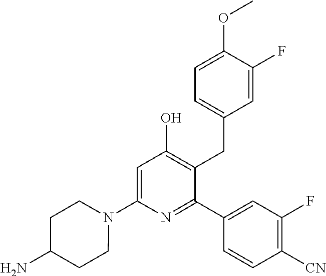 [SMILES: COc1ccc(Cc2c(O)cc(N3CCC(N)CC3)nc2-c2ccc(C#N)c(F)c2)cc1F] | ¹H NMR (400 MHz, Methanol-d₄) δ ppm 7.82 (t, J = 7.2 Hz, 1H), 7.41 (d, J = 9.6 Hz, 1H), 7.32 (d, J = 8.0 Hz, 1H), 6.82 (t, J = 8.6 Hz, 1H), 6.60 (dd, J = 12.4, 1.8 Hz, 1H), 6.53 (d, J = 8.4 Hz, 1H), 6.47 (d, J = 3.2 Hz, 1H), 4.03 (d, J = 13.4 Hz, 2H), 3.71 (s, 3H), 3.65 (s, 1H), 3.38 (dt, J = 3.8, 1.8 Hz, 2H), 3.16 (s, 2H), 2.16- 2.00 (m, 2H), 1.68 (d, J = 12.4 Hz, 2H). | 451.2 |
| 58 | 4-(6-(4-aminopiper- idin-1-yl)-3-(4- fluoro-3-hydroxy- phenyl)-4-hydroxy- pyridin-2-yl)-2- fluorobenzonitrile | 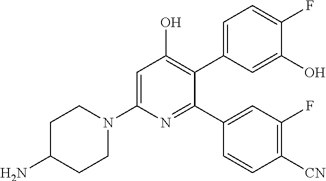 [SMILES: N#Cc1ccc(-c2nc(N3CCC(N)CC3)cc(O)c2-c2ccc(F)c(O)c2)cc1F] | ¹H NMR (400 MHz, DMSO-d₆) δ ppm 7.73 (dd, J = 8.2, 6.8 Hz, 1H), 7.33-7.23 (m, 1H), 7.15 (dd, J = 8.2, 1.6 Hz, 1H), 6.97 (dd, J = 11.4, 8.4 Hz, 1H), 6.69 (dd, J = 8.6, 2.2 Hz, 1H), 6.47-6.34 (m, 1H), 6.30 (s, 1H), 4.10 (d, J = 13.0 Hz, 2H), 2.99-2.71 (m, 3H), 1.76 (dd, J = 12.8, 3.8 Hz, 2H), 1.30-1.12 (m, 2H). | 423.2 |
| 59 | 4-(6-(4-aminopi- peridin-1-yl)-3-(4- chloro-3-hydroxy- phenyl)-4-hydroxy- pyridin-2-yl)-2- fluorobenzonitrile | 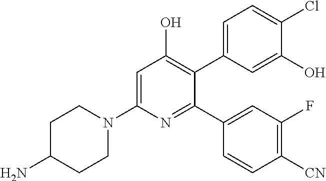 [SMILES: N#Cc1ccc(-c2nc(N3CCC(N)CC3)cc(O)c2-c2ccc(Cl)c(O)c2)cc1F] | ¹H NMR (400 MHz, DMSO-d₆) δ ppm 7.73 (t, J = 7.4 Hz, 1H), 7.30 (dd, J = 10.8, 1.4 Hz, 1H), 7.14 (dd, J = 8.2, 1.6 Hz, 2H), 6.72 (d, J = 1.8 Hz, 1H), 6.42 (dd, J = 8.2, 2.0 Hz, 1H), 6.27 (s, 1H), 4.10 (dt, J = 13.5, 3.7 Hz, 2H), 2.98-2.74 (m, 3H), 1.81-1.68 (m, 2H), 1.23 (td, J = 15.2, 14.6, 7.8 Hz, 2H). | 439.1 |
| 60 | 4-(6-(4-aminopiper- idin-1-yl)-4- hydroxy-3- (3-hydroxy- 4-methylphenyl) pyridin-2-fluoro- benzonitrile | 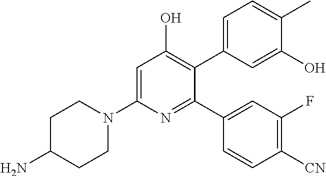 [SMILES: Cc1ccc(-c2c(O)cc(N3CCC(N)CC3)nc2-c2ccc(C#N)c(F)c2)cc1O] | ¹H NMR (400 MHz, DMSO-d₆) δ ppm 7.70 (dd, J = 8.2, 70 Hz, 1H), 7.27 (dd, J = 11.0, 1.4 Hz, 1H), 7.17 (dd, J = 8.2, 1.5 Hz, 1H), 6.89 (d, J = 7.8 Hz, 1H), 6.54 (d, J = 1.8 Hz, 1H), 6.34 (dd, J = 7.6, 1.6 Hz, 1H), 6.29 (s, 1H), 4.09 (dd, J = 13.2, 3.8 Hz, 2H), 2.92-2.72 (m, 3H), 2.08 (s, 3H), 1.79-1.64 (m, 2H), 1.29-1.14 (m, 2H). | 419.2 |
| 61 | 4-(6-((3-aminopro- pyl)(meth- yl)amino)- 3-(4-chloro-3- hydroxyphenyl)-4- hydroxypyridin- 2-yl)-2-fluoroben- zonitrile | 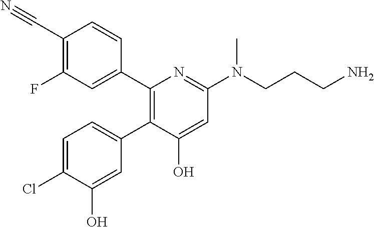 [SMILES: CN(CCCN)c1cc(O)c(-c2ccc(Cl)c(O)c2)c(-c2ccc(C#N)c(F)c2)n1] | ¹H NMR (400 MHz, Metanol-d₄) δ ppm 7.56 (dd, J = 8.0, 6.8 Hz, 1H), 7.29-7.17 (m, 2H), 7.11 (d, J = 8.1 Hz, 1H), 6.68 (d, J = 1.8 Hz, 1H), 6.48 (dt, J = 8.2, 2.0 Hz, 1H), 6.14- 5.98 (m, 1H), 3.71 (t, J = 6.2 Hz, 2H), 2.97 (s, 3H), 2.82-2.75 (m, 2H), 1.91 (p, J = 6.3 Hz, 2H). | 427.1 |
| 62 | 4-(6-((3-aminopro- pyl)(methyl)amino)- 3-(4-chloro-3- hydroxyphenyl)-4- hydroxypyridin- 2-yl)-2-fluoroben- zonitrile | 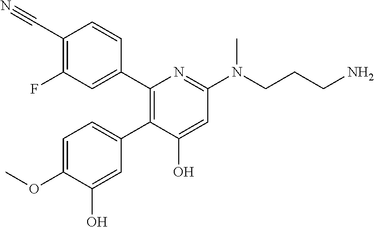 [SMILES: COc1ccc(-c2c(O)cc(N(C)CCCN)nc2-c2ccc(C#N)c(F)c2)cc1O] | ¹H NMR (400 MHz, Methanol-d₄) δ ppm 7.53 (dd, J = 8.4, 6.6 Hz, 1H), 7.25-7.18 (m, 2H), 7.14-6.89 (m, 1H), 6.78 (d, J = 8.2 Hz, 1H), 6.60 (d, J = 2.0 Hz, 1H), 6.50-6.43 (m, 1H), 3.82 (s, 3H), 3.73-3.68 (m, 2H), 2.97 (s, 3H), 2.76 (t, J = 6.2 Hz, 2H), 1.89 (q, J = 6.2 Hz, 2H). | 423.2 |
| 63 | 4-(6-((3-aminopro- pyl)(methyl)amino)- 4-hydroxy-3-(3- hydroxy-4-methyl- phenyl)pyridin-2- yl)-2-fluorobenzo- nitrile hydrochloride | 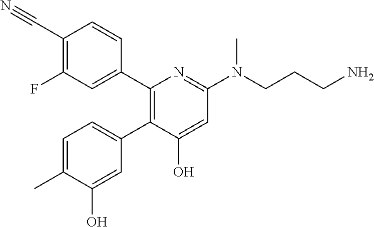 [SMILES: Cc1ccc(-c2c(O)cc(N(C)CCCN)nc2-c2ccc(C#N)c(F)c2)cc1O] | ¹H NMR (400 MHz, DMSO-d₆) δ ppm 9.16 (s, 1H), 8.05 (s, 3H), 7.78 (t, J = 7.4 Hz, 1H), 7.55-7.40 (m, 1H), 7.23 (d, J = 8.2 Hz, 1H), 6.90 (d, J = 7.8 Hz, 1H), 6.55 (d, J = 1.6 Hz, 1H), 6.46 (s, 1H), 6.33 (dd, J = 7.4, 1.6 Hz, 1H), 3.65 (d, J = 6.8 Hz, 2H), 3.06 (s, 3H), 2.82 (h, J = 6.0 Hz, 2H), 2.06 (s, 3H), 1.92 (p, J = 7.2 Hz, 2H) | 407.2 |

### Example 64

### Preparation of 4-(6-(4-aminopiperidin-1-yl)-3-(3-fluoro-4-methoxyphenyl)amino)-4-hydroxypyridin-2-yl)-2-flu orobenzonitrile hydrochloride

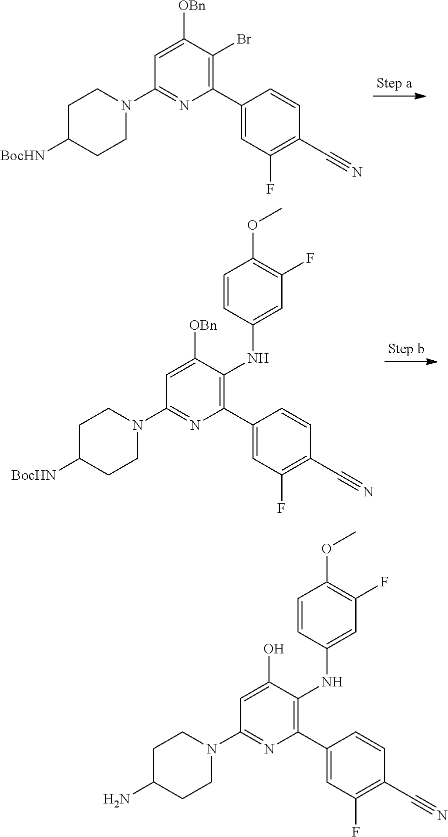

### Step a): Preparation of tert-butyl 1-(4-(benzyloxy)-6-(4-cyano-3-fluorophenyl)-5-(3-fluoro-4-methoxyphenyl)amino)pyridin-2-yl-piperidin-4-yl)carbamate

### Tert-butyl

(1-(4-(benzyloxy)-5-bromo-6-(4-cyano-3-fluorophenyl)pyridin-2-yl)piperidin-4-yl)carbamate (200 mg, 0.34 mmol), 3-fluoro-4-methoxyaniline (96 mg, 0.68 mmol), Pd₂(dba)₃ (30 mg, 0.03 minol), 2-dicyclohexylphosphine-2′,6′-diisopropoxy-1,1′-biphenyl (32 mg, 0.07 mmol), and sodium tert-butoxide (96 mg, 1.02 mmol) are weighed, added with anhydrous toluene (2 mL), and heated and reacted in microwaves for 1.5 h under nitrogen protection. When LC-MS shows that the reaction is completed, the reaction solution is extracted with ethyl acetate (20 mL×3), washed with saturated saline (20 mL), dried with anhydrous sodium sulfate, filtered, and concentrated; and the residue is purified by silica gel chromatography (eluent: petroleum ether/ethyl acetate=1/1) to obtain tert-butyl 1-(4-(benzyloxy)-6-(4-cyano-3-fluorophenyl)-5-(3-fluoro-4-methoxyphenyl)amino)pyridin-2-yl-piperidin-4-yl)carbamate with a yield of 27.2%.

ESI-MS m/z: 642.3[M+H]⁺.

### Step b): Preparation of 4-(6-(4-aminopiperidin-1-yl)-3-(3-fluoro-4-methoxyphenyl)amino)-4-hydroxypyridin-2-yl)-2-flu orobenzonitrile hydrochloride

### Tert-butyl

(1-(4-(benzyloxy)-6-(4-cyano-3-fluorophenyl)-5-(3-fluoro-4-methoxyphenyl)amino)pyridin-2-yl-piperidin-4-yl)carbamate (63 mg, 0.10 mmol) is weighed and added to a sealed tube, and added with trifluoroacetic acid (2 mL), and warmed to 70° C. in an oil bath and reacted for 16 h under nitrogen protection. When LC-MS shows that the reaction is completed, the reaction solution is concentrated to dryness, added with a methanol (2 mL) solution, and then purified by a pre-HPLC preparation column (separation method 1) to obtain 4-(6-(4-aminopiperidin-1-yl)-3-(3-fluoro-4-methoxyphenyl)amino)-4-hydroxypyridin-2-yl)-2-flu orobenzonitrile hydrochloride with a yield of 10.7%.

¹H NMR (400 MHz, Methanol-d₄) δ ppm 7.84-7.75 (m, 1H), 7.63 (d, J=9.8 Hz, 1H), 7.55 (dd, J=8.2, 1.6 Hz, 1H), 6.82 (t, J=9.0 Hz, 1H), 6.67 (d, J=2.4 Hz, 1H), 6.39-6.25 (m, 2H), 4.16 (dt, J=14.0, 2.8 Hz, 2H), 3.74 (s, 3H), 3.50 (tt, J=12.0, 4.2 Hz, 1H), 3.37-3.33 (m, 1H), 3.28 (d, J=2.8 Hz, 1H), 2.20 (dd, J=12.8, 3.8 Hz, 2H), 1.83 (qd, J=12.4, 3.8 Hz, 2H).

ESI-MS m/z: 452.2[M+H]⁺.

### Example 65

### Preparation of 4-(6-(4-aminopiperidin-1-yl)-4-hydroxy-3-(3-hydroxy-4-methylphenyl)-5-methylpyridin-2-yl)-2-fluorobenzonitrile hydrochloride

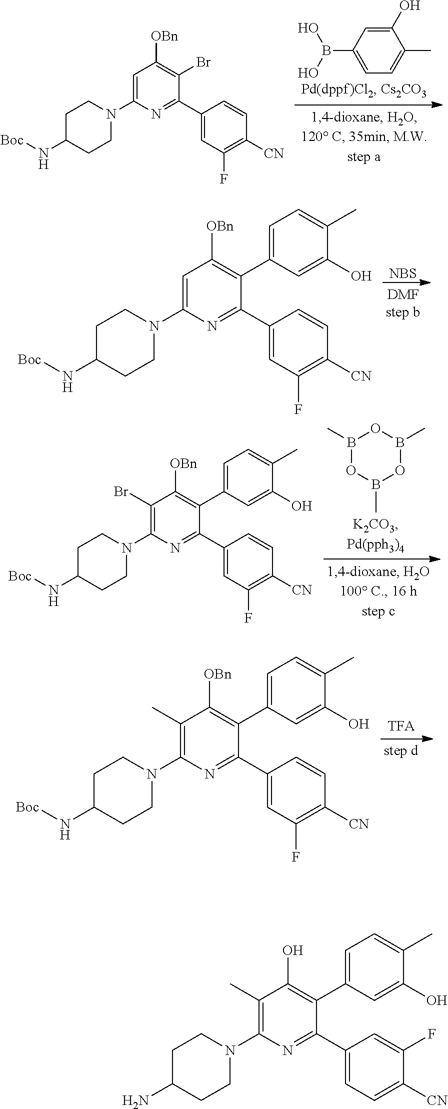

### Step a): Preparation of tert-butyl 1-(4-(benzyloxy)-6-(4-cyano-3-fluorophenyl)-5-(3-hydroxy-4-methoxyphenyl)pyridin-2-yl)piper idin-4-yl)carbamate

### Tert-butyl

1-(4-(benzyloxy)-5-bromo-6-(4-cyano-3-fluorophenyl)pyridin-2-yl)piperidin-4-yl)carbamate (300 mg, 0.518 mmol), (3-hydroxy-4-methylphenyl)boronic acid (118 mg, 0.76 mmol), Cs₂CO₃ (337 mg, 1.03 mmol), and Pd(dppf)Cl₂ (38 mg, 0.05 mmol) are dissolved in 8 mL of dioxane and added with 0.8 mL of water; and the mixed reaction solution is purged by nitrogen blowing and protected with nitrogen. The mixture reacts in a microwave reactor at 120° C. for 30 min; when LC-MS shows that the reaction is completed, a crude product is obtained by concentration, and the residue is purified by silica gel chromatography (eluent: petroleum ether/ethyl acetate=10/3) to obtain tert-butyl 1-(4-(benzyloxy)-6-(4-cyano-3-fluorophenyl)-5-(3-hydroxy-4-methoxyphenyl)pyridin-2-yl)piper idin-4-yl)carbamate with a yield of 90.4%.

¹H NMR (400 MHz, DMSO-d₆) δ ppm 9.83 (s, 1H), 7.76 (t, J=7.6 Hz, 1H), 7.41-7.17 (m, 9H), 6.55 (d, J=17.8 Hz, 2H), 5.24-5.09 (m, 2H), 4.44-4.19 (m, 2H), 3.51 (s, 2H), 3.02-2.85 (m, 2H), 2.09 (s, 3H), 1.99 (s, 1H), 1.88-1.73 (m, 2H), 1.40 (s, 9H).

ESI-MS m/z: 609.3[M+H]⁺.

### Step b): Preparation of tert-butyl (1-(4-(benzyloxy)-3-bromo-6-(4-cyano-3-fluorophenyl)-5-(3-hydroxy-4-methoxyphenyl)pyridin-2-yl)piperidin-4-yl)carbamate

### Tert-butyl

1-(4-(benzyloxy)-6-(4-cyano-3-fluorophenyl)-5-(3-hydroxy-4-methylphenyl)pyridin-2-yl)piperid in-4-yl)carbamate (200 mg, 0.038 mmol) is dissolved in N,N-dimethylformamide (10 mL), added with NBS (140 mg, 0.039 mmol) at 0° C. and stirred. When LS-MS shows that the reaction is completed. The reaction solution is quenched with water (20 mL) and extracted with ethyl acetate (20 mL×2); organic layers are combined and then washed with saturated saline (15 mL×2), dried with anhydrous sodium sulfate, filtered, and concentrated under a reduced pressure to obtain a residue; and the residue is purified by silica gel chromatography (eluent: petroleum ether/ethyl acetate=2/1) to obtain tert-butyl (1-(4-(benzyloxy)-3-bromo-6-(4-cyano-3-fluorophenyl)-5-(3-hydroxy-4-methoxyphenyl)pyridin-2-yl)piperidin-4-yl)carbamate with a yield of 64.5%.

ESI-MS(m/z)=687.2[M+H]⁺.

### Step c): Preparation of tert-butyl 1-(4-(benzyloxy)-6-(4-cyano-3-fluorophenyl)-5-(3-hydroxy-4-methoxyphenyl)-3-methylpyridin-2-yl)piperidin-4-yl)carbamate

### Tert-butyl

(1-(4-(benzyl)-3-bromo-6-(4-cyano-3-fluorophenyl)-5-(3-hydroxy-4-methylphenyl)pyridin-2-yl) piperidin-4-yl)carbamate (50 mg, 0.07 mmol), 2,4,6-trimethyl-1,3,5,2,4,6-trioxatriborinane (100 uL, 0.2 mmol), K₂CO₃ (30 mg, 0.22 mmol), and Pd(dppf)Cl₂ (8 mg, 0.014 mmol) are dissolved in 4 mL of dioxane; and the mixed reaction solution is purged by nitrogen blowing and protected with nitrogen. The mixture reacts at 100° C. for 16 h; when TLC and LC-MS show that the reaction is completed, a crude product is obtained by concentration, and the crude product is purified by silica gel chromatography (eluent: petroleum ether/ethyl acetate=5/3) to obtain tert-butyl 1-(4-(benzyloxy)-6-(4-cyano-3-fluorophenyl)-5-(3-hydroxy-4-methoxyphenyl)-3-methylpyridin-2-yl)piperidin-4-yl)carbamate with a yield of 70.2%.

ESI-MS m/z: 623.3[M+H]⁺.

### Step d): Preparation of 4-(6-(4-aminopiperidin-1-yl)-4-hydroxy-3-(3-hydroxy-4-methylphenyl)-5-methylpyridin-2-yl)-2-fluorobenzonitrile hydrochloride

### Tert-butyl

1-(4-(benzyloxy)-6-(4-cyano-3-fluorophenyl)-5-(3-hydroxy-4-methylphenyl)-3-methylpyridin-2-yl)piperidin-4-yl)carbamate (67 mg, 0.107 mmol) is added to 3 mL of trifluoroacetic acid solution, and reacted at 75° C. for 16 h; and when LC-MS shows that the reaction is completed, the solvent is dried by spinning; and the crude product is subjected to Prep-HPLC preparation (separation method 1) to obtain 4-(6-(4-aminopiperidin-1-yl)-4-hydroxy-3-(3-hydroxy-4-methylphenyl)-5-methylpyridin-2-yl)-2-fluorobenzonitrile hydrochloride with a yield of 20.4%.

¹H NMR (400 MHz, Methanol-d₄) δppm 7.55 (t, J=7.4 Hz, 1H), 7.29 (d, J=9.8 Hz, 1H), 7.14 (d, J=8.0 Hz, 1H), 6.75 (s, 1H), 6.47 (s, 1H), 6.18 (s, 1H), 4.05 (d, J=13.6 Hz, 2H), 3.36 (ddt, J=11.4, 8.6, 4.2 Hz, 1H), 3.21 (d, J=13.2 Hz, 2H), 2.06 (dd, J=13.0, 3.8 Hz, 2H), 1.96 (s, 3H), 1.82 (s, 3H), 1.69 (qd, J=12.4, 4.2 Hz, 2H).

ESI-MS m/z: 433.2[M+H]⁺.

### Example 66

### Preparation of 2-(4-aminopiperidin-1-yl)-4-(4-cyano-3-fluorophenyl)-5-(3-hydroxy-4-methoxyphenyl)nicotinon itrile hydrochloride

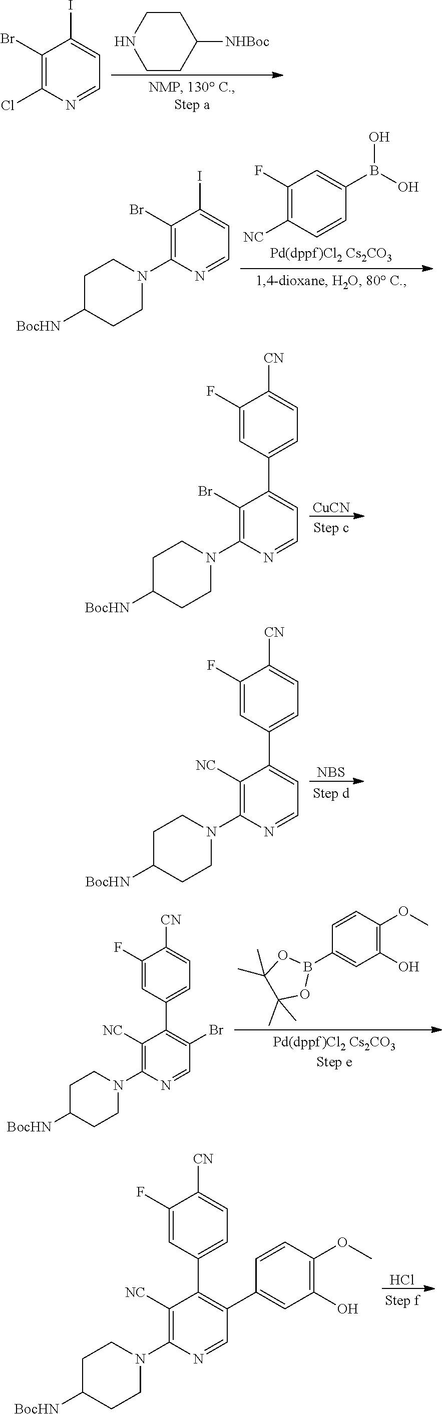

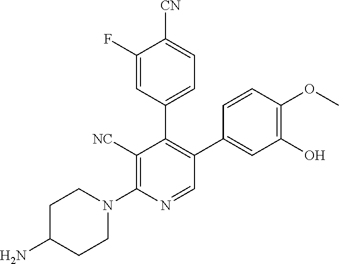 [SMILES: COc1ccc(-c2cnc(N3CCC(N)CC3)c(C#N)c2-c2ccc(C#N)c(F)c2)cc1O]

### Step a): Preparation of tert-butyl(4-(3-bromo-4-iodopyridin-2-yl)cyclohexyl)carbamate

3-bromo-2-chloro-4-iodopyridine (1.0 g, 3.155 mmol), tert-butyl piperidin-4-yl-carbamate (0.946 g, 4.732 mmol) and NMP (10 mL) are added to a microwave reactor and reacted at 130° C. for 1 h while stirring. When the reaction is completed, the reaction solution is added with water (20 mL), and extracted with ethyl acetate (20 mL×3); organic phase is combined, and washed with saturated saline (20 mL×2); the organic phase is concentrated under a reduced pressure to dryness; and the residue is purified by silica gel chromatography (eluent: petroleum ether/ethyl acetate=3/1) to obtain tert-butyl(4-(3-bromo-4-iodopyridin-2-yl)cyclohexyl)carbamate with a yield of 43.0%.

¹H NMR (400 MHz, DMSO-d₆) δppm 7.88 (d, J=5.0 Hz, 1H), 7.53 (d, J=5.0 Hz, 1H), 3.66-3.49 (m, 2H), 3.41 (s, 1H), 2.87-2.68 (m, 2H), 1.90-1.71 (m, 2H), 1.52 (qd, J=11.8, 3.6 Hz, 2H), 1.39 (s, 9H).

ESI-MS(m/z)=482.0[M+H]⁺.

### Step b): Preparation of tert-butyl (1-(3-bromo-4-(4-cyano-3-fluorophenyl)pyridin-2-yl)piperidin-4-yl)carbamate

Tert-butyl (4-(3-bromo-4-iodopyridin-2-yl)cyclohexyl)carbamate (653 mg, 5.5 mmol), (4-cyano-3-fluorophenyl)boronic acid (336 mg, 5.5 mmol), Cs₂CO₃ (0.885 g, 2.714 mmol), Pd(dppf)Cl₂ (100 mg, 0.136 mmol), 1.4-dioxane (10 mL) and H₂O (2.5 mL) are dissolved in a reaction flask, and reacted at 100° C. for 1 h while stirring. The reaction is concentrated under a reduced pressure to dryness; the residue is purified by silica gel chromatography (eluent: petroleum ether/ethyl acetate=3/1) to obtain tert-butyl (1-(3-bromo-4-(4-cyano-3-fluorophenyl)pyridin-2-yl)piperidin-4-yl)carbamate with a yield of 47.0%.

¹H NMR (400 MHz, DMSO-d₆) δ 8.30 (d, J=4.8 Hz, 1H), 8.17-7.95 (m, 1H), 7.69 (dd, J=10.2, 1.6 Hz, 1H), 7.47 (dd, J=8.0, 1.6 Hz, 1H), 7.00 (d, J=4.8 Hz, 1H), 3.66 (d, J=12.6 Hz, 2H), 3.44 (s, 1H), 2.86 (t, J=11.8 Hz, 2H), 1.84 (d, J=12.4 Hz, 2H), 1.56 (tt, J=12.4, 6.2 Hz, 2H), 1.39 (s, 9H).

ESI-MS(m/z)=475.1 [M+H]⁺.

### Step c): Preparation of tert-butyl (1-(3-cyano-4-(4-cyano-3-fluorophenyl)pyridin-2-yl)piperidin-4-yl)carbamate

Tert-butyl(1-(3-bromo-4-(4-cyano-3-fluorophenyl)pyridin-2-yl)piperidin-4-yl)carbamate (303 mg, 0.638 mmol), cuprous cyanide (115 mg, 1.276 mmol) and DMSO (10 mL) are added to a reaction flask and reacted at 80° C. for 12 h while stirring. When the reaction is completed, the reaction solution is added with water (40 mL), and extracted with ethyl acetate (40 mL×3); organic phase is combined, and washed with saturated saline (40 mL×2), and concentrated under a reduced pressure to dryness; and the residue is purified by silica gel chromatography (eluent: petroleum ether/ethyl acetate=1/1) to obtain tert-butyl (1-(3-cyano-4-(4-cyano-3-fluorophenyl)pyridin-2-yl)piperidin-4-yl)carbamate with a yield of 21.0%. ESI-MS(m/z)=422.2[M+H]⁺.

### Step d): Preparation of tert-butyl(1-(4-(benzyloxy)-5-bromo-6-(4-cyano-3-fluorophenyl)pyridin-2-yl)piperidin-3-yl)(methyl)carbamate

Tert-butyl (1-(3-cyano-4-(4-cyano-3-fluorophenyl)pyridin-2-yl)piperidin-4-yl)carbamate (57 mg, 0.134 mmol) and DMF (2 mL) are added to a reaction flask, added with NBS in batches (29 mg, 0.161 mmol) under ice bath agitation, and reacted while stirring at room temperature for 30 min. When the reaction is completed, the reaction solution is added with water (40 mL), and extracted with ethyl acetate (40 mL×3); organic phase is combined, and washed with saturated saline (40 mL×2), and concentrated under a reduced pressure to dryness; and the residue is purified by silica gel chromatography (eluent: petroleum ether/ethyl acetate=2/1) to obtain tert-butyl (1-(5-3-cyano-4-(4-cyano-3-fluorophenyl)pyridin-2-yl)piperidin-4-yl)carbamate with a yield of 77.0%.

ESI-MS(m/z)=500.2[M+H]⁺.

### Step e): Preparation of tert-butyl (1-(3-cyano-4-(4-cyano-3-fluorophenyl)-5-(3-hydroxy-4-methoxyphenyl)pyridin-2-yl)piperidin-4-yl)carbamate

### Tert-butyl

(1-(5-bromo-3-cyano-4-(4-cyano-3-fluorophenyl)pyridin-2-yl)piperidin-4-yl)carbamate (52 mg, 0.103 mmol), 2-methoxy-5-(4,4,5,5-tetramethyl-1,3,2-dioxaborolane-2-yl)phenol (39 mg, 0.155 mmol), Cs₂CO₃ (67 mg, 0.206 mmol), Pd(dppf)Cl₂ (7 mg, 0.010 mmol), 1.4-Dioxane (4 mL) and H₂O (1 mL) are added to a reaction flask, and reacted at 120° C. for 1 h while stirring. The reaction is concentrated under a reduced pressure to dryness; the residue is purified by silica gel chromatography (eluent: petroleum ether/ethyl acetate=1/1) to obtain tert-butyl (1-(3-cyano-4-(4-cyano-3-fluorophenyl)-5-(3-hydroxy-4-methoxyphenyl)pyridin-2-yl)piperidin-4-yl)carbamate with a yield of 67.0%.

ESI-MS(m/z)=544.3[M+H]⁺.

### Step f): Preparation of 2-(4-aminopiperidin-1-yl)-4-(4-cyano-3-fluorophenyl)-5-(3-hydroxy-4-methoxyphenyl)nicotinon itrile hydrochloride

### Tert-butyl

1-(3-cyano-4-(4-cyano-3-fluorophenyl)-5-(3-hydroxy-4-methoxyphenyl)pyridin-2-yl)piperidin-4-yl)carbamic acid (38 mg, 0.069 mmol) is added to a reaction flask, then added with 4M hydrogen chloride (ethyl acetate solution, 2.5 mL) and stirred at room temperature for 1 h; a large amount of solids precipitated, which are concentrated under a reduced pressure to obtain a crude product; and the crude product is purified by Prep-HPLC (separation method 1) to obtain 2-(4-aminopiperidin-1-yl)-4-(4-cyano-3-fluorophenyl)-5-(3-hydroxy-4-methoxyphenyl)nicotinon itrile hydrochloride with a yield of 52.5%.

¹H NMR (400 MHz, DMSO-d₆) δ 9.01 (s, 1H), 8.45 (s, 1H), 8.17 (s, 3H), 7.96 (dd, J=8.0, 6.8 Hz, 1H), 7.67 (dd, J=10.2, 1.6 Hz, 1H), 7.28 (dd, J=8.0, 1.6 Hz, 1H), 6.89-6.70 (m, 1H), 6.47 (d, J=7.0 Hz, 2H), 4.25 (d, J=13.6 Hz, 2H), 3.72 (s, 3H), 3.42 (d, J=5.0 Hz, 1H), 3.16 (t, J=12.4 Hz, 2H), 2.05 (d, J=11.0 Hz, 2H), 1.75-1.61 (m, 2H).

ESI-MS(m/z)=444.2 [M+H]⁺.

Compounds in Examples 67-86 are prepared according to the synthesis method of Example 66 (separation method for the compounds: free alkali, hydrochloride and formate are separately prepared according to separation methods 4, 1 and 3, respectively), and their structures and characterization data are as follows:

| | | | | MS |
|---|---|---|---|---|
| Examples | Chemical name | Structure | ¹H NMR | (M + H)⁺ |
| 67 | 2-(4- aminopiperidin-1- yl)-4-(4- cyano-3-fluoro phenyl)-5-(3- hydroxy-4- methylphenyl) nicotinonitrile hydrochloride | 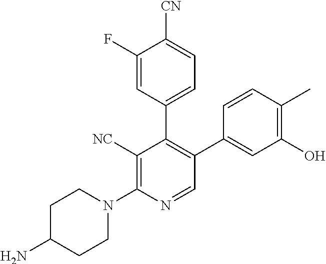 [SMILES: Cc1ccc(-c2cnc(N3CCC(N)CC3)c(C#N)c2-c2ccc(C#N)c(F)c2)cc1O] | ¹H NMR (400 MHz, DMSO-d₆) δ ppm 9.36 (s, 1H), 8.45 (s, 1H), 8.17 (br, 3H), 7.96 (t, J = 7.4 Hz, 1H), 7.71 (dd, J = 10.2, 1.4 Hz, 1H), 7.26 (dd, J = 8.0, 1.4 Hz, 1H), 6.96 (d, J = 7.6 Hz, 1H), 6.63 − 6.27 (m, 2H), 4.27 (d, J = 13.4 Hz, 2H), 3.35 (s, 1H), 3.17 (t, J = 12.0 Hz, 2H), 2.05 (s, 5H), 1.69 (tt, J = 12.8, 6.4 Hz, 2H). | 428.2 |
| 68 | 2-(4-aminopiperidin-1- yl)-4-(4-cyano- 3-fluoro phenyl)-5-(3- fluoro-4- methoxyphenyl) nicotinonitrile hydrochloride | 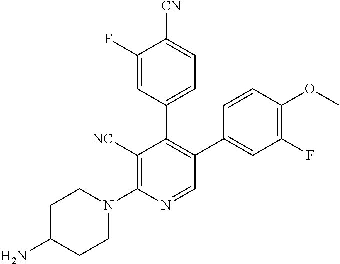 [SMILES: COc1ccc(-c2cnc(N3CCC(N)CC3)c(C#N)c2-c2ccc(C#N)c(F)c2)cc1F] | ¹H NMR (400 MHz, DMSO-d₆) δ ppm 8.51 (s, 1H), 8.24 − 7.88 (m, 4H), 7.69 (dd, J = 10.1, 1.6 Hz, 1H), 7.32 (dd, J = 8.0, 1.6 Hz, 1H), 7.13 − 6.93 (m, 2H), 6.85 − 6.69 (m, 1H), 4.29 (d, J = 13.4 Hz, 2H), 3.80 (s, 3H), 3.40 (s, 1H),3.18 (t, J = 12.4 Hz, 2H), 2.12 − 1.94 (m, 2H), 1.75 − 1.56 (m, 2H). | 446.2 |
| 69 | 2-(4-aminopiperidin-1- yl)-4-(4-cyano-3- fluorophenyl)-5- (4-methoxyphenyl) nicotinonitrile hydrochloride | 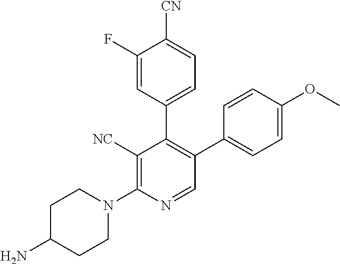 [SMILES: COc1ccc(-c2cnc(N3CCC(N)CC3)c(C#N)c2-c2ccc(C#N)c(F)c2)cc1] | ¹H NMR (400 MHz, DMSO-d₆ + D₂O) δ ppm 8.49 (s, 1H), 7.97 (dd, J = 8.0, 6.8 Hz, 1H), 7.67 (dd, J = 10.0, 1.5 Hz, 1H), 7.29 (dd, J = 8.0, 1.5 Hz, 1H), 7.10 − 6.90 (m, 2H), 6.90 − 6.72 (m, 2H), 4.27 (d, J = 13.4 Hz, 2H), 3.71 (s, 3H), 3.33 (d, J = 9.0 Hz, 1H), 3.15 (d, J = 11.6 Hz, 2H), 2.16 − 1.96 (m, 2H), 1.68 (tt, J = 13.2, 6.6 Hz, 2H). | 428.2 |
| 70 | 2-(4-aminopiperidin-1- yl)-4-(4-cyano- 3-fluoro phenyl)-5-(3- hydroxy-4- (trifluoromethyl) phenyl) nicotinonitrile hydrochloride | 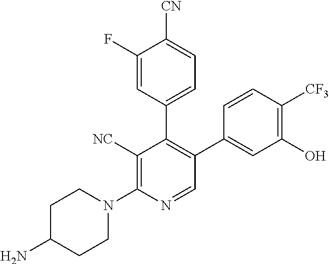 [SMILES: N#Cc1ccc(-c2c(-c3ccc(C(F)(F)F)c(O)c3)cnc(N3CCC(N)CC3)c2C#N)cc1F] | ¹H NMR (400 MHz, Methanol-d₄) δ ppm 8.38 (s, 1H), 7.76 − 7.62 (m, 1H), 7.33 (dd, J = 28.2, 8.8 Hz, 2H), 7.19 − 6.99 (m, 1H), 6.57 (d, J = 7.4 Hz, 2H), 4.38 (d, J = 13.4 Hz, 2H), 3.46 − 3.28 (m, 1H), 3.14 (t, J = 12.8 Hz, 2H), 2.08 (d, J = 12.2 Hz, 2H), 1.80 − 1.60 (m, 2H). | 482.2 |
| 71 | 2-(4-aminopiperidin-1- yl)-4-(4-cyano-3- fluorophenyl)- 5-(5-fluoro-3- methylbenzo[d] isoxazol- 6-yl)nicotinonitrile | 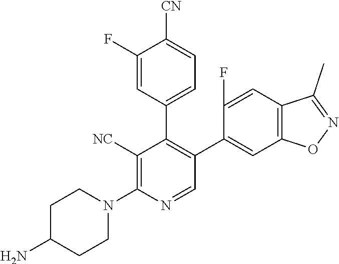 [SMILES: Cc1noc2cc(-c3cnc(N4CCC(N)CC4)c(C#N)c3-c3ccc(C#N)c(F)c3)c(F)cc12] | ¹H NMR (400 MHz, DMSO-d₆) δ ppm 8.51 (s, 1H), 7.91 (t, J = 7.4 Hz, 1H), 7.76 (dd, J = 24.8, 7.8 Hz, 2H), 7.62 (d, J = 8.8 Hz, 1H), 7.32 (d, J = 8.0 Hz, 1H), 4.39 − 4.17 (m, 2H), 3.28 − 3.20 (m, 2H), 2.90 (dq, J = 9.4, 4.8, 4.2 Hz, 1H), 2.51 (s, 3H), 1.97 − 1.81 (m, 2H), 1.37 (q, J = 13.2, 11.8 Hz, 2H). | 471.2 |
| 72 | 2-(4-aminopiperidin-1- yl)-4-(4-cyano-3- fluorophenyl)-5- (3-fluoro-4- methylphenyl) nicotinitrile | 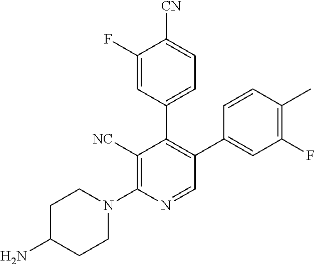 [SMILES: Cc1ccc(-c2cnc(N3CCC(N)CC3)c(C#N)c2-c2ccc(C#N)c(F)c2)cc1F] | ¹H NMR (400 MHz, DMSO-d₆) δ ppm 8.48 (s, 1H), 7.97 (dd, J = 8.0, 6.8 Hz, 1H), 7.68 (dd, J = 10.0, 1.6 Hz, 1H), 7.31 (dd, J = 8.0, 1.6 Hz, 1H), 7.15 (t, J = 8.0 Hz, 1H), 6.95 (dd, J = 10.8, 1.6 Hz, 1H), 6.73 (dd, J = 7.8, 1.6 Hz, 1H), 4.22 (d, J = 13.0 Hz, 2H), 3.24 − 3.12 (m, 2H), 2.95 (dt, J = 10.4, 6.0 Hz, 1H), 2.17 (d, J = 1.8 Hz, 3H), 1.88 (dd, J = 13.0, 3.8 Hz, 2H), 1.40 (p, J = 14.6, 12.8 Hz, 2H). | 430.2 |
| 73 | 2-(5-amino-2- azabicyclo [2.2.1]heptane-2-yl)-4- (4-cyano-3- fluorophenyl)- 5-(3-hydroxy-4- methoxyphenyl) nicotinitrile | 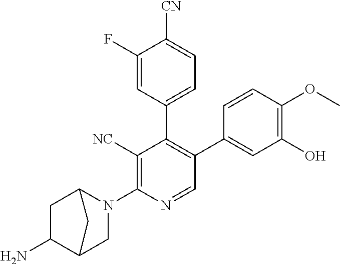 [SMILES: COc1ccc(-c2cnc(N3CC4CC3CC4N)c(C#N)c2-c2ccc(C#N)c(F)c2)cc1O] | ¹H NMR (400 MHz, DMSO-d₆) δ ppm 9.02 (s, 1H), 8.36 (s, 1H), 8.00 (s, 1H), 7.91 − 7.05 (m, 2H), 6.84 (d, J = 8.2 Hz, 1H), 6.49 (dd, J = 7.4, 2.4 Hz, 2H), 5.05 − 4.68 (m, 2H), 4.06 (d, J = 9.6 Hz, 0.5H), 3.76 (s, 4H), 3.51 (dt, J = 9.4, 4.2 Hz, 0.5H), 3.27 (d, J = 9.8 Hz, 0.5H), 3.17 − 3.04 (m, 0.5H), 2.17 − 2.07 (m, 1H), 2.00 (d, J = 9.8 Hz, 0.5H), 1.83 (d, J = 10.2 Hz, 0.5H), 1.68 (dd, J = 33.8, 9.8 Hz, 1H), 1.38 − | 456.2 |
| | | | 1.18 (m, 1H). | |
| 74 | 2-(8-amino-3- azabicyclo[3.2.1] octane-3-yl)-4-(4- cyano-3-fluorophenyl)- 5-(3-hydroxy-4- methoxyphenyl) nicotinonitrile | 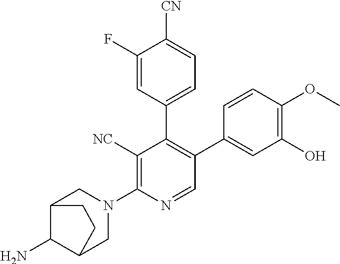 [SMILES: COc1ccc(-c2cnc(N3CC4CCC(C3)C4N)c(C#N)c2-c2ccc(C#N)c(F)c2)cc1O] | ¹H NMR (400 MHz, DMSO-d₆) δ ppm 8.99 (s, 1H), 8.39 (s, 1H), 7.95 (dd, J = 8.0, 6.8 Hz, 1H), 7.66 (dd, J = 10.2, 1.6 Hz, 1H), 7.27 (dd, J = 8.0, 1.6 Hz, 1H), 6.80 (d, J = 8.2 Hz, 1H), 6.47 (d, J = 8.2 Hz, 2H), 3.93 (s, 2H), 3.71 (s, 5H), 3.08 (t, J = 4.6 Hz, 1H), 1.98 (s, 2H), 1.78 − 1.56 (m, 4H). | 470.2 |
| 75 | 2-(3-amino- 9-azabicyclo [3.3.1]nonan-9-yl)-4- (4-cyano-3- fluorophenyl)- 5-(3-hydroxy-4- methoxyphenyl) nicotinonitrile | 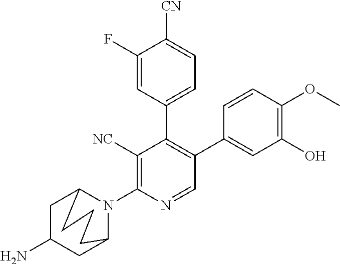 [SMILES: COc1ccc(-c2cnc(N3C4CCCC3CC(N)C4)c(C#N)c2-c2ccc(C#N)c(F)c2)cc1O] | ¹H NMR (400 MHz, DMSO-d₆) δ ppm 8.38 (s, 1H), 7.95 (dd, J = 8.0, 6.8 Hz, 1H), 7.68 (dd, J 10.2, 1.6 Hz, 1H), 7.27 (dd, J = 8.0, 1.4 Hz, 1H), 6.80 (d, J = 8.2 Hz, 1H), 6.64 − 6.29 (m, 2H), 4.71 (d, = 10.6 Hz, 2H), 3.71 (s, 3H), 2.56 (dd, J = 11.6, 5.8 Hz, 1H), 2.27 (td, J = 11.8, 5.6 Hz, 2H), 1.84 (s, 1H), 1.74 (s, 2H), 1.54 (t, J = 16.4 Hz, 3H), 1.28 (td, J = 12.4, 3.8 Hz, 2H) | 484.2 |
| 76 | 2-(5-amino-2- azabicyclo[2.2.2] octane-2-yl)-4-(4- cyano-3-fluorophenyl)- 5-(3-hydroxy-4- methoxyphenyl) nicotinonitrile | 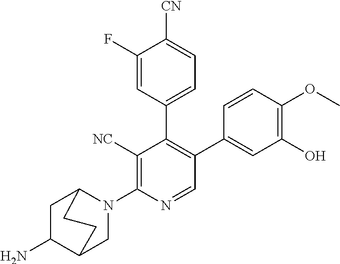 [SMILES: COc1ccc(-c2cnc(N3CC4CCC3CC4N)c(C#N)c2-c2ccc(C#N)c(F)c2)cc1O] | ¹H NMR (400 MHz, DMSO-d₆) δ ppm 8.98 (s, 1H), 8.33 (d, J = 2.0 Hz, 1H), 7.95 (t, J = 7.4 Hz, 1H), 7.63 (s, 1H), 7.26 (s, 1H), 6.91 − 6.66 (m, 1H), 6.45 (dd, J = 8.2, 1.6 Hz, 2H), 4.71 − 4.46 (m, 1H), 4.17 (d, J = 10.8 Hz, 0.5H), 3.71 (s, 4H), 3.54 (d, J = 10.6 Hz, 0.5H), 3.14 (s, 1H), 2.28 (t, J = 11.6 Hz, 1H), 2.10 (s, 1H), 1.94 − 1.71 (m, 3H), 1.65 − 1.57 (m, 1H), 1.51 − 1.36 (m, 1H). | 470.2 |
| 77 | 2-(4-amino-3- fluoropiperidin- 1-yl)-4-(4-cyano- 3-fluorophenyl)-5-(3- hydroxy-4- methoxyphenyl) nicotinonitrile | 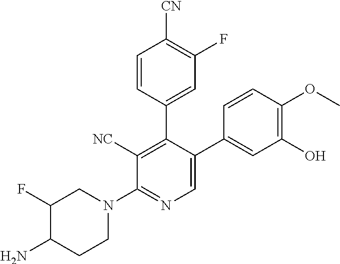 [SMILES: COc1ccc(-c2cnc(N3CCC(N)C(F)C3)c(C#N)c2-c2ccc(C#N)c(F)c2)cc1O] | ¹H NMR (400 MHz, Methanol-d₄) δ 8.34 (s, 1H), 7.64 (dd, J = 8.0, 6.6 Hz, 1H), 7.28 (dd, J = 9.8, 1.5 Hz, 1H), 7.14 (dd, J = 8.0, 1.5 Hz, 1H), 6.72 (d, J = 8.8 Hz, 1H), 6.54 − 6.28 (m, 2H), 4.46 − 4.21 (m, 2H), 4.15 (d, J = 13.6 Hz, 1H), 3.71 (s, 3H), 3.17 − 2.98 (m, 2H), 2.92 (tdd, J = 13.2, 8.5, 4.8 Hz, 1H), 1.96 (ddd, J = 10.6, 5.2, 2.5 Hz, 1H), 1.53 (qd, J = 12.0, 4.3 Hz, 1H). | 462.2 |
| 78 | (R)-2-(3- aminopiperidin- 1-yl)-4-(4-cyano- 3-f1uorophenyl)- 5-(3-hydroxy-4- methoxyphenyl) nicotinonitrile | 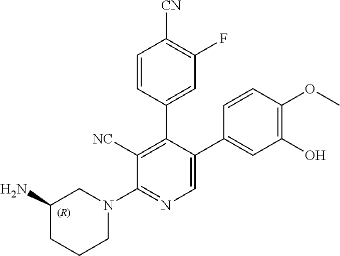 | ¹H NMR (400 MHz, Methanol-d₄) δ 8.30 (s, 1H), 7.64 (dd, J = 8.0, 6.6 Hz, 1H), 7.27 (dd, J = 9.8, 1.5 Hz, 1H), 7.13 (dd, J = 8.0, 1.5 Hz, 1H), 6.71 (d, J = 8.8 Hz, 1H), 6.53 − 6.28 (m, 2H), 4.12 (dd, J = 8.4, 2.0 Hz, 1H), 4.02 (d, J = 13.2 Hz, 1H), 3.71 (s, 3H), 3.16 − 3.00 (m, 1H), 2.97 − 2.82 (m, 2H), 1.95 (dd, J = 10.6, 6.4 Hz, 1H), 1.79 (dt, J = 13.6, 3.9 Hz, 1H), 1.64 (qt, J = 14.6, 3.9 Hz, 1H), 1.42 − 1.22 (m, 1H). | 444.2 |
| 79 | 4-(4-cyano-3- fluorophenyl)-5-(3- hydroxy-4- methoxyphenyl)- 2-(4-(methylamino) piperidin-1-yl) nicotinonitrile hydrochloride | 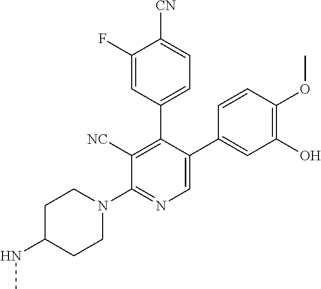 | ¹H NMR (400 MHz, DMSO-d₆) δ ppm 8.85 (s, 3H), 8.45 (s, 1H), 7.97 (dd, J = 8.0, 6.8 Hz, 1H), 7.68 (dd, J = 10.2, 1.4 Hz, 1H), 7.28 (dd, J = 8.0, 1.6 Hz, 1H), 6.98 − 6.66 (m, 1H), 6.47 (d, J = 7.4 Hz, 2H), 4.29 (d, J = 13.4 Hz, 2H), 3.71 (s, 3H), 3.38 − 3.19 (m, 1H), 3.12 (t, J = 12.6 Hz, 2H), 2.59 (t, J = 5.4 Hz, 3H), 2.17 (d, J = 12.0 Hz, 2H), 1.76 − 1.57 (m, 2H). | 458.2 |
| 80 | 2-(4-amino-4- methylpiperidin- 1-yl)-4-(4-cyano- 3-fluorophenyl)-5-(3- hydroxy-4- methoxyphenyl) )nicotinonitrile hydrochloride | 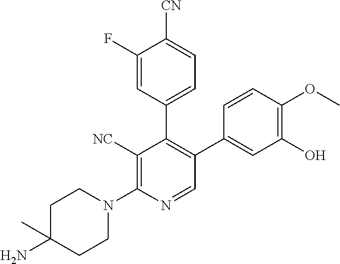 [SMILES: COc1ccc(-c2cnc(N3CCC(C)(N)CC3)c(C#N)c2-c2ccc(C#N)c(F)c2)cc1O] | ¹H NMR (400 MHz, DMSO-d₆) δ 9.05 (s, 1H), 8.46 (s, 1H), 8.15 (s, 3H), 7.98 (dd, J = 8.0, 6.8 Hz, 1H), 7.69 (dd, J = 10.2, 1.6 Hz, 1H), 7.28 (dd, J = 8.2, 1.6 Hz, 1H), 6.85-6.78 (m, 1H), 6.48 (d, J = 7.2 Hz, 2H), 4.02 (dd, J = 11.8, 7.2 Hz, 2H), 3.72 (s, 3H), 3.51-3.40 (m, 2H), 1.91 (td, J = 11.0, 9.6, 6.0 Hz, 2H), 1.86-1.76 (m, 2H), 1.42 (s, 3H). | 458.2 |
| 81 | 2-(3-amino- 8-azabicyclo [3.2.1]octane-8-yl)- 4-(4-cyano-3- fluorophenyl)-5-(3- hydroxy-4- methoxyphenyl) nicotinonitrile | 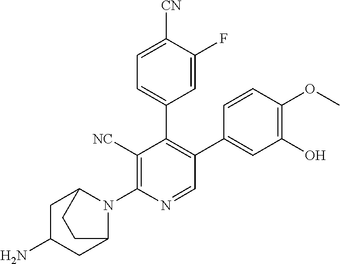 [SMILES: COc1ccc(-c2cnc(N3C4CCC3CC(N)C4)c(C#N)c2-c2ccc(C#N)c(F)c2)cc1O] | ¹H NMR (400 MHz, DMSO-d₆) δ ppm 8.35 (s, 1H), 7.94− 7.86 (m, 1H), 7.58 (dd, J = 10.0, 1.6 Hz, 1H), 7.24 (dd, J = 8.0, 1.6 Hz, 1H), 6.78 (d, J = 8.4 Hz, 1H), 6.50 − 6.40 (m, 2H), 4.98 −4.84 (m, 2H), 3.87 (s, 1H), 3.68 (s, 3H), 1.98-1.75 (m, 6H), 1.59 (dt, J = 27.6, 12.0 Hz, 2H). | 470.2 |
| 82 | 2-(3-aminoazetidin- 1-yl)-4-(4-cyano-3- fluorophenyl)-5-(3- hydroxy-4- methoxyphenyl) nicotinonitrile | 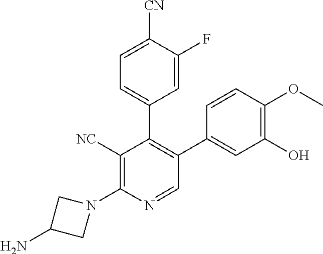 [SMILES: COc1ccc(-c2cnc(N3CC(N)C3)c(C#N)c2-c2ccc(C#N)c(F)c2)cc1O] | ¹H NMR (400 MHz, DMSO-d₆) δ ppm 8.98 (s, 1H), 8.32 (s, 1H), 7.95 (dd, J = 8.0, 6.8 Hz, 1H), 7.58 (dd, J = 10.0, 1.6 Hz, 1H), 7.23 (dd, J = 8.0, 1.6 Hz, 1H), 6.84 − 6.74 (m, 1H), 6.46 − 6.37 (m, 2H), 4.44 (dd, J = 8.8, 6.8 Hz, 2H), 3.89 (dd, J = 8.6, 5.6 Hz, 2H), 3.83 (t, J = 6.2 Hz, 1H), 3.70 (s, 3H), 2.18 (s, 2H). | 416.2 |
| 83 | 6-(4-aminopiperidin-1- yl)-4-(4-cyano-3- fluorophenyl)-1′- methyl-2′-oxo-1′,2′- dihydro-[3,4′- bipyridine]-5- carbonitrile hydrochloride | 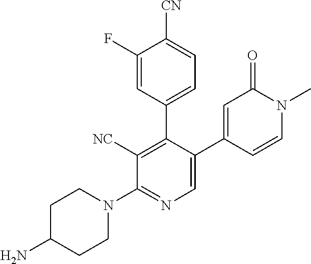 [SMILES: Cn1ccc(-c2cnc(N3CCC(N)CC3)c(C#N)c2-c2ccc(C#N)c(F)c2)cc1=O] | ¹H NMR (400 MHz, Methanol-d₄) δ ppm 8.20 (s, 1H), 7.71 (d, J = 6.8 Hz, 1H), 7.56 − 7.39 (m, 1H), 7.22 (d, J = 9.4 Hz, 1H), 7.01 (d, J = 7.8 Hz, 1H), 6.59 (d, J = 1.8 Hz, 1H), 6.48 (dd, J = 6.8, 1.8 Hz, 1H), 4.86 − 4.78 (m, 2H), 4.24 (d, J = 13.4 Hz, 2H), 3.14 (tt, J = 11.3, 3.8 Hz, 1H), 3.02 − 2.87 (m, 3H), 1.89 − 1.75 (m, 2H), 1.45 (qd, J = 12.4, 3.9 Hz, 2H). | 429.2 |
| 84 | 2-(4-aminopiperidin-1- yl)-4-(4- cyano-3- fluorophenyl)-5- (2-methyl-2H- indazol-5-yl) nicotinonitrile hydrochloride | 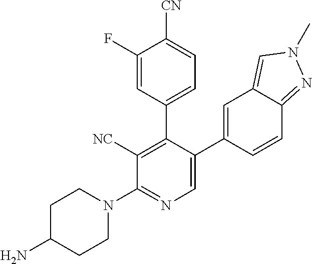 [SMILES: Cn1cc2cc(-c3cnc(N4CCC(N)CC4)c(C#N)c3-c3ccc(C#N)c(F)c3)ccc2n1] | ¹H NMR (400 MHz, Methanol-d₄) δ ppm 8.69 (s, 1H), 8.34 (s, 1H), 7.71 (s, 1H), 7.53 (s, 1H), 7.48 − 7.35 (m, 1H), 7.35 − 7.05 (m, 3H), 4.38 − 4.27 (m, 2H), 4.21 (s, 3H), 3.21 − 3.04 (m, 3H), 2.21 − 1.92 (m, 2H), 1.79 − 1.56 (m, 2H). | 452.2 |
| 85 | 2-(4-aminopiperidin-1- yl)-4-(4-cyano-3-fluoro phenyl)-5-(3- hydroxy-4- morpholinophenyl) nicotinonitrile hydrochloride | 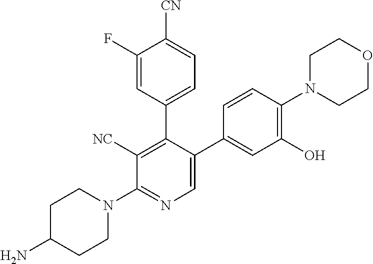 [SMILES: N#Cc1ccc(-c2c(-c3ccc(N4CCOCC4)c(O)c3)cnc(N3CCC(N)CC3)c2C#N)cc1F] | ¹H NMR (400 MHz, Methanol-d₄) δ 8.37 (s, 1H), 7.69 (dd, J = 7.8, 6.6 Hz, 1H), 7.51 − 7.42 (m, 1H), 7.35 (d, J = 9.8 Hz, 1H), 7.20 (d, J = 7.8 Hz, 1H), 6.72 (d, J = 7.2 Hz, 2H), 4.39 (d, J = 13.2 Hz, 2H), 3.98 (t, J = 4.8 Hz, 4H), 3.64 (s, 4H), 3.42 − 3.31 (m, 1H), 3.14 (t, J = 12.8 Hz, 2H), 2.08 (d, J = 12.4 Hz, 2H), 1.72 (tt, J = 12.8, 6.4 Hz, 2H). | 499.2 |
| 86 | 2-(4-aminopiperidin-1- phenyl)-5- (3-hydroxy-4- (2-hydroxy-2-methyl propoxy)phenyl) nicotinonitrile hydrochloride | 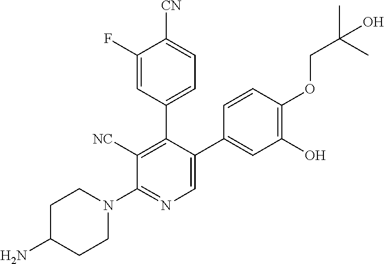 [SMILES: CC(C)(O)COc1ccc(-c2cnc(N3CCC(N)CC3)c(C#N)c2-c2ccc(C#N)c(F)c2)cc1O] | ¹H NMR (400 MHz, DMSO-d₆) δ 8.88 (br, 1H), 8.52 (s, 1H), 8.15 (br, 3H), 8.04 (t, J = 7.4 Hz, 1H), 7.74 (d, J = 10.0 Hz, 1H), 7.36 (d, J = 8.0 Hz, 1H), 6.84 (d, J = 8.2 Hz, 1H), 6.57 (d, J = 2.2 Hz, 1H), 6.50 (dd, J = 8.2, 2.2 Hz, 1H), 4.32 (d, J = 13.4 Hz, 2H), 3.71 (s, 2H), 3.48 − 3.34 (m, 1H), 3.23 (t, J = 12.6 Hz, 2H), 2.12 (dd, J = 12.8, 3.9 Hz, 2H), 1.83 − 1.65 (m, 2H), 1.24 (s, 6H). | 502.2 |

### Example 87

### Preparation of 4-(6-(4-aminopiperidin-1-yl)-3-(3-fluoro-4-methoxyphenyl)-4-methoxypyridin-2-yl)-2-fluoroben zonitrile

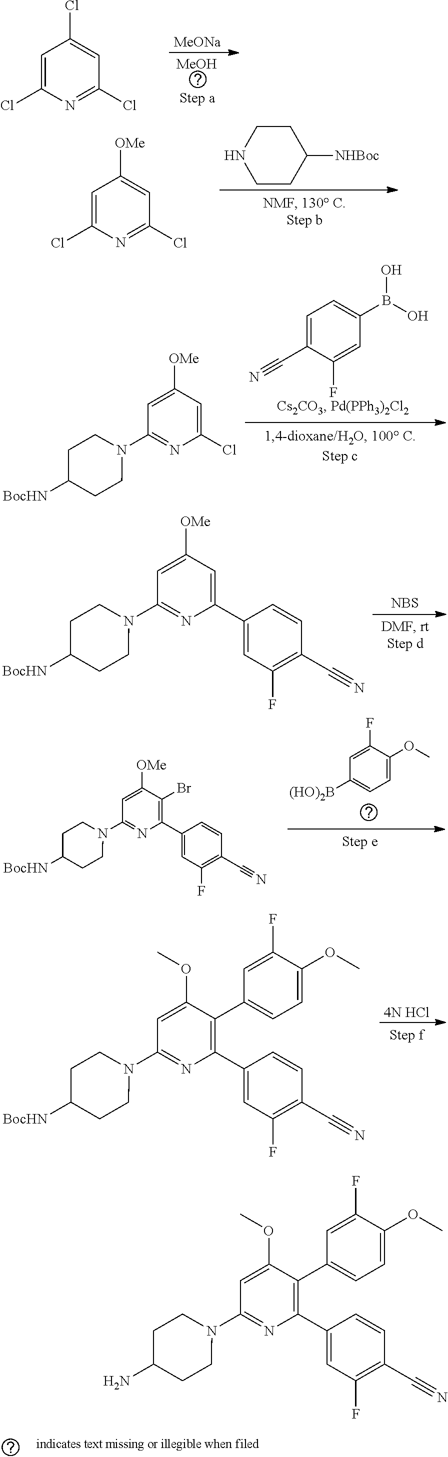

### Step a): Preparation of 2,6-dichloro-4-methoxypyridine

4-(benzyloxy)-2,6-dichloropyridine (2 g, 11.049 mmol) and MeOH (20 mL) are added to a reaction flask and reacted while stirring at room temperature for 16 h. When the reaction is completed, the reaction solution is added with water (100 mL), and extracted with ethyl acetate (100 mL×3); organic phase is combined, and washed with saturated saline (100 mL×2); the organic phase is concentrated under a reduced pressure to dryness; and the residue is purified by silica gel chromatography (eluent: petroleum ether/ethyl acetate=2/1) to obtain 2,6-dichloro-4-methoxypyridine with a yield of 60.5%.

¹H NMR (400 MHz, Chloroform-d) δppm 6.79 (s, 2H), 3.87 (s, 3H).

ESI-MS(m/z)=178.6[M+H]⁺.

### Step b): Preparation of tert-butyl (1-(6-chloro-4-methoxypyridin-2-yl)piperidin-4-yl)carbamate

2,6-dichloro-4-methoxypyridine (1.2 g, 6.629 mmol), tert-butyl piperidin-4-yl-carbamate (2.7 g, 13.258 mmol) and NMP (15 mL) are added to a microwave reactor and reacted at 130° C. for 2 h while stirring. When the reaction is completed, the reaction solution is added with water (100 mL), and extracted with ethyl acetate (100 mL×3); organic phase is combined, and washed with saturated saline (100 mL×2), and concentrated under a reduced pressure to dryness; and the residue is purified by silica gel chromatography (eluent: petroleum ether/ethyl acetate=1/1) to obtain tert-butyl (1-(6-chloro-4-methoxypyridin-2-yl)piperidin-4-yl)carbamate with a yield of 52.0%.

ESI-MS(m/z)=342.5[M+H]⁺.

### Step c): Preparation of tert-butyl (1-(6-(4-cyano-3-fluorophenyl)-4-methoxypyridin-2-yl)piperidin-4-yl)carbamate

Tert-butyl (1-(6-chloro-4-methoxypyridin-2-yl)piperidin-4-yl)carbamate (1.2 g, 3.447 mmol), (4-cyano-3-fluorophenyl)boronic acid (853 mg, 5.170 mmol), Cs₂CO₃ (2.2 g, 6.894 mmol), Pd(dppf)Cl₂ (253 mg, 0.345 mmol), 1.4-dioxane (10 mL) and H₂O (2.5 mL) are added to a reaction flask, and reacted at 120° C. for 1 h while stirring. The reaction solution is concentrated under a reduced pressure to dryness; and the residue is purified by silica gel chromatography (eluent: petroleum ether/ethyl acetate=2/3) to obtain tert-butyl (1-(6-(4-cyano-3-fluorophenyl)-4-methoxypyridin-2-yl)piperidin-4-yl)carbamate with a yield of 73.0%.

¹H NMR (400 MHz, DMSO-d₆) δ 8.12 (dd, J=15.4, 9.8 Hz, 2H), 7.02 (d, J=1.8 Hz, 1H), 6.83 (d, J=7.8 Hz, 1H), 6.40 (s, 1H), 4.33 (d, J=13.2 Hz, 2H), 3.86 (m, 3H), 3.31 (s, 3H), 2.94 (t, J=12.4 Hz, 2H), 1.80 (d, J=12.0 Hz, 2H), 1.39 (s, 9H).

ESI-MS(m/z)=427.3 [M+H]⁺.

### Step d): Preparation of tert-butyl (1-(5-bromo-6-(4-cyano-3-fluorophenyl)-4-methoxypyridin-2-yl)piperidin-4-yl)carbamate

Tert-butyl (1-(6-(4-cyano-3-fluorophenyl)-4-methoxypyridin-2-yl)piperidin-4-yl)carbamate (1.1 g, 2.516 mmol) and DMF (20 mL) are added to a reaction flask, added with NBS in batches (448 mg, 2.516 mmol) in an ice bath while stirring, and reacted at room temperature for 30 min while stirring. When the reaction is completed, the reaction solution is added with water (40 mL), and extracted with ethyl acetate (40 mL×3); organic phase is combined, and washed with saturated saline (40 mL×2), and concentrated under a reduced pressure to dryness; and the residue is purified by silica gel chromatography (eluent: petroleum ether/ethyl acetate=2/3) to obtain tert-butyl (1-(5-bromo-6-(4-cyano-3-fluorophenyl)-4-methoxypyridin-2-yl)piperidin-4-yl)carbamate with a yield of 72.0%. E

ESI-MS m/z)=505.2 [M+H]⁺.

### Step e): Preparation of tert-butyl (1-(6-(4-cyano-3-fluorophenyl)-5-(3-fluoro-4-methoxyphenyl)-4-methoxypyridin-2-yl)piperidin-4-yl)carbamate

Tert-butyl(1-(5-bromo-6-(4-cyano-3-fluorophenyl)-4-methoxypyridin-2-yl)piperidin-4-yl)carbam ate (200 mg, 0.396 mmol), (3-fluoro-4-methoxyphenyl)boronic acid (101 mg, 0.595 mmol), Cs₂CO₃ (258 mg, 0.702 mmol), Pd(dppf)Cl₂ (29 mg, 0.039 mmol), 1.4-dioxane (8 mL) and H₂O (2 mL) are added to a reaction flask, and reacted at 120° C. for 1 h while stirring. The reaction solution is concentrated under a reduced pressure to dryness; and the residue is purified by silica gel chromatography (eluent: petroleum ether/ethyl acetate=2/3) to obtain tert-butyl (1-(6-(4-cyano-3-fluorophenyl)-5-(3-fluoro-4-methoxyphenyl)-4-methoxypyridin-2-yl)piperidin-4-yl)carbamate with a yield of 87.0%.

ESI-MS(m/z)=551.3[M+H]⁺.

### Step f): Preparation of 4-(6-(4-aminopiperidin-1-yl)-3-(3-fluoro-4-methoxyphenyl)-4-methoxypyridin-2-yl)-2-fluoroben zonitrile

### Tert-butyl

1-(6-(4-cyano-3-fluorophenyl)-5-(3-fluoro-4-methoxyphenyl)-4-methoxypyridin-2-yl)piperidin-4-yl)carbamate (190 mg, 0.345 mmol) is added to a reaction flask, then added with 4M hydrogen chloride (ethyl acetate solution, 2.5 mL) and stirred at room temperature for 1 h; a large amount of solids precipitated, and are concentrated under a reduced pressure to obtain a crude product; and the crude product is purified by Prep-HPLC (separation method 4) to obtain 4-(6-(4-aminopiperidin-1-yl)-3-(3-fluoro-4-methoxyphenyl)-4-methoxypyridin-2-yl)-2-fluoroben zonitrile with a yield of 51.0%.

¹H NMR (400 MHz, DMSO-d₆) δppm 7.74 (t, J=7.4 Hz, 1H), 7.30 (d, J=10.8 Hz, 1H), 7.14 (d, J=7.8 Hz, 1H), 7.07-6.84 (m, 2H), 6.72 (d, J=8.4 Hz, 1H), 6.49 (s, 1H), 4.29 (dt, J=13.4, 3.8 Hz, 2H), 3.79 (d, J=6.8 Hz, 6H), 3.02-2.85 (m, 2H), 2.80 (tt, J=9.8, 4.0 Hz, 1H), 1.88-1.51 (m, 4H).

ESI-MS(m/z)=451.5 [M+H]⁺.

Compounds in Examples 88-100 are prepared according to the synthesis method of Example 87 (separation method for the compounds: free alkali, hydrochloride and formate are separately prepared according to separation methods 4, 1 and 3, respectively), and their structures and characterization data are as follows:

| | | | | MS |
|---|---|---|---|---|
| Examples | Chemical name | Structure | ¹H NMR | (M + H)⁺ |
| 88 | 4-(6-(3-amino-8- azabicyclo [3.2.1]octane-8-yl)-4- (benzyloxy)-3-(5-fluoro-3- methylbenzo[d]isoxazol- 6-yl)pyridin-2-yl)-2- fluorobenzonitrile | 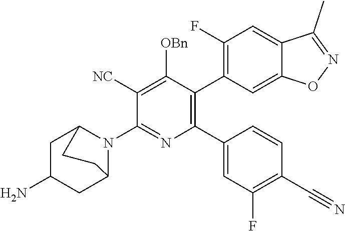 [SMILES: Cc1noc2cc(-c3c(-c4ccc(C#N)c(F)c4)nc(N4C5CCC4CC(N)C5)c(C#N)c3OCc3ccccc3)c(F)cc12] | ¹H NMR (400 MHz, DMSO-d₆) δ ppm 7.78 − 7.63 (m, 2H), 7.57 (d, J = 5.4 Hz, 1H), 7.40 7.22 (m, 6H), 7.12 (d, J = 8.2 Hz, 1H), 6.53 (s, 1H), 5.21 (s, 2H), 4.59 (s, 2H), 3.23 − 3.08 (m, 1H), 2.54 (s, 3H), 1.94 (s, 3H), 1.73 (dd, J = 28.8, 10.6 Hz, 5H). | 578.2 |
| 89 | 4-(6-(4-aminopiperidin- 1-yl)-3-(3-hydroxy-4- methoxyphenyl)-4- methoxypyridin-2-yl)-2- fluorobenzonitrile | 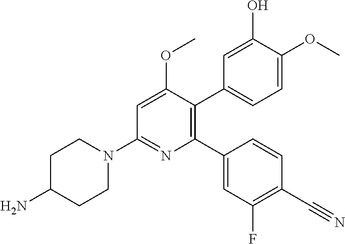 [SMILES: COc1ccc(-c2c(OC)cc(N3CCC(N)CC3)nc2-c2ccc(C#N)c(F)c2)cc1O] | ¹H NMR (400 MHz, DMSO-d₆) δ ppm 8.85 (s, 1H), 7.73 (dd, J = 8.0, 7.0 Hz, 1H), 7.42 − 7.10 (m, 2H), 6.78 (d, J = 8.4 Hz, 1H), 6.68 − 6.43 (m, 2H), 6.37 (dd, J = 8.2, 2.0 Hz, 1H), 4.29 (d, J = 13.0 Hz, 2H), 3.76 (d, J = 15.0 Hz, 6H), 3.01 − 2.87 (m, 2H), 2.87 − 2.74 (m, 1H), 1.78 (d, J = 11.8 Hz, 2H), 1.23 (q, J = 10.2, 9.0 Hz, 2H). | 449.2 |
| 90 | 4-(6-(4-aminopiperidin-1- yl)-3-(5-fluoro-3-methyl benzo[d]isoxazol-6-yl)- 4-methoxypyridin- 2-yl)-2- fluorobenzonitrile | 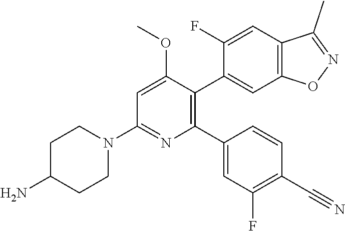 [SMILES: COc1cc(N2CCC(N)CC2)nc(-c2ccc(C#N)c(F)c2)c1-c1cc2onc(C)c2cc1F] | ¹H NMR (400 MHz, DMSO-d₆) δ ppm 7.72 (dd, J = 8.0, 6.8 Hz, 1H), 7.66 (d, J = 8.6 Hz, 1H), 7.52 (d, J = 5.4 Hz, 1H), 7.35 (dd, J = 10.4, 1.6 Hz, 1H), 7.12 (dd, J = 8.0, 1.6 Hz, 1H), 6.55 (s, 1H), 4.34 (dd, J = 14.0, 3.8 Hz, 2H), 3.80 (s, 3H), 2.99 (ddd, J = 13.8, 9.8, 3.2 Hz, 2H), 2.90 − 2.77 (m, 1H), 2.50 (s, 3H), 1.85 − 1.76 (m, 2H), 1.33 − 1.20 (m, 2H). | 476.2 |
| 91 | 4-(6-(4-aminopiperidin-1- yl)-3-(4-fluoro-3- hydroxyphenyl)- 4-methoxypyridin- 2-yl)-2- fluorobenzonitrile | 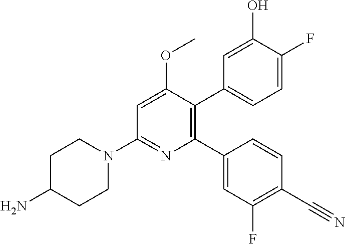 [SMILES: COc1cc(N2CCC(N)CC2)nc(-c2ccc(C#N)c(F)c2)c1-c1ccc(F)c(O)c1] | ¹H NMR (400 MHz, DMSO-d₆) δ ppm 7.74 (dd, J = 8.0, 7.0 Hz, 1H), 7.29 (dd, J = 10.8, 1.6 Hz, 1H), 7.15 (dd, J = 8.0, 1.6 Hz, 1H), 6.99 (dd, J = 11.4, 8.4 Hz, 1H), 6.66 (dd, J = 8.8, 2.2 Hz, 1H), 6.51 (s, 1H), 6.41 (ddd, J = 8.4, 4.4, 2.2 Hz, 1H), 4.39 − 4.29 (m, 2H), 3.79 (s, 3H), 2.94 (td, J = 12.4, 11.4, 2.6 Hz, 3H), 1.87 − 1.78 (m, 2H), 1.37 − 1.21 (m, 2H). | 437.2 |
| 92 | 4-(6-(4-aminopiperidin- 1-yl)-3-(3,4-difluoro-5- hydroxyphenyl)-4- methoxypyridin-2-yl)- 2-fluorobenzonitrile | 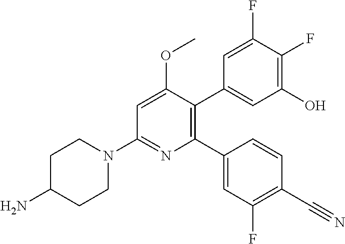 [SMILES: COc1cc(N2CCC(N)CC2)nc(-c2ccc(C#N)c(F)c2)c1-c1cc(O)c(F)c(F)c1] | ¹H NMR (400 MHz, DMSO-d₆) δ ppm 7.77 (dd, J = 8.0, 6.8 Hz, 1H), 7.35 (dd, J = 10.8, 1.6 Hz, 1H), 7.14 (dd, J = 8.0, 1.6 Hz, 1H), 6.63 − 6.47 (m, 2H), 6.41 (dt, J = 7.4, 1.8 Hz, 1H), 4.34 (dt, J = 13.2, 3.8 Hz, 2H), 3.80 (s, 3H), 3.03 − 2.86 (m, 3H), 1.92 − 1.76 (m, 2H), 1.39 − 1.20 (m, 2H). | 455.2 |
| 93 | 4-(6-(4-aminopiperidin-1- yl)-3-(3-cyano-4- methoxyphenyl)-4- methoxypyridin- 2-yl)-2-fluorobenzonitrile | 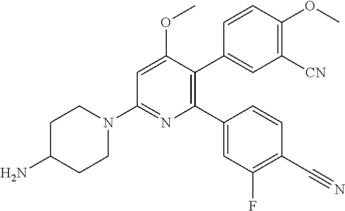 [SMILES: COc1ccc(-c2c(OC)cc(N3CCC(N)CC3)nc2-c2ccc(C#N)c(F)c2)cc1C#N] | ¹H NMR (400 MHz, DMSO-d₆) δ ppm 7.75 (dd, J = 8.0, 6.8 Hz, 1H), 7.43 (d, J = 2.2 Hz, 1H), 7.34 (dd, J = 10.8, 1.6 Hz, 1H), 7.26 (dd, J = 8.8, 2.2 Hz, 1H), 7.11 (dd, J = 8.2, 1.4 Hz, 2H), 6.52 (s, 1H), 4.34 (dd, J = 13.6, 3.8 Hz, 2H), 3.88 (s, 3H), 3.80 (s, 3H), 3.02 − 2.85 (m, 3H), 1.90 − 1.76 (m, 2H), 1.35 − 1.19 (m, 2H). | 458.2 |
| 94 | 4,4′-(6-(4-aminopiperidin- 1-yl)-4-methoxypyridin- 2,3-diyl)bis(2- fluorobenzonitrile) | 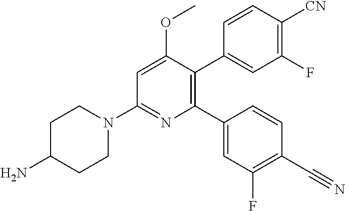 [SMILES: COc1cc(N2CCC(N)CC2)nc(-c2ccc(C#N)c(F)c2)c1-c1ccc(C#N)c(F)c1] | ¹H NMR (400 MHz, DMSO-d₆) δ ppm 7.75 (ddd, J = 8.4, 7.0, 1.8 Hz, 2H), 7.34 (ddd, J = 14.8, 10.6, 1.6 Hz, 2H), 7.08 (dd, J = 8.0, 1.6 Hz, 1H), 7.02 (dd, J = 8.0, 1.6 Hz, 1H), 6.50 (s, 1H), 4.34 (dd, J = 10.8, 6.6 Hz, 2H), 3.82 (s, 3H), 2.97 (ddd, J = 13.8, 11.6, 2.8 Hz, 2H), 2.88 (dt, J = 10.4, 5.8Hz, 1H), 1.80 | 446.2 |
| | | | (dd, J = 12.6, 3.8 Hz, | |
| | | | 2H), 1.36 − 1.18 (m, 2H). | |
| 95 | 4-(6-(4-aminopiperidin-1- yl)-3-(3-hydroxy-4- methylphenyl)-4- methoxypyridin-2-yl)-2- fluorobenzonitrile hydrochloride | 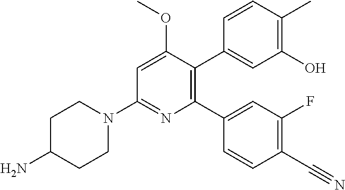 [SMILES: COc1cc(N2CCC(N)CC2)nc(-c2ccc(C#N)c(F)c2)c1-c1ccc(C)c(O)c1] | ¹H NMR (400 MHz, Methanol-d₄) δ ppm 7.71 (dd, J = 8.0, 6.6 Hz, 1H), 7.50 (d, J = 9.8 Hz, 1H), 7.35 (d, J = 8.0 Hz, 1H), 6.95 (d, J = 7.6 Hz, 1H), 6.80 (s, 1H), 6.53 (d, J = 1.4 Hz, 1H), 6.45 − 6.38 (m, 1H), 4.37 (d, J = 13.4 Hz, 2H), 4.03 (s, 3H), 3.57 (ddt, J = 11.2, 7.6, 3.8 Hz, 1H), 3.43 (t, J = | 433.2 |
| | | | 12.8 Hz, 2H), 2.26 (dd, | |
| | | | J = 13.6, 3.8 Hz, 2H), 2.12 | |
| | | | (s, 3H), 1.90 (qd, J = | |
| | | | 12.4, 3.8 Hz, 2H). | |
| 96 | 2-fluoro-4-(3-(3-hydroxy- 4-methoxyphenyl)-4- methoxy-6-(piperazinyl) pyridin-2-yl)benzonitrile hydrochloride | 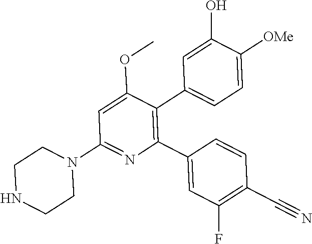 [SMILES: COc1ccc(-c2c(OC)cc(N3CCNCC3)nc2-c2ccc(C#N)c(F)c2)cc1O] | ¹H NMR (400 MHz, DMSO-d₆) δ ppm 9.20 (d, J = 26.4 Hz, 1H), 7.74 (t, J = 7.6 Hz, 1H), 7.34 (dd, J = 10.8, 1.4 Hz, 1H), 7.18 (dd, J = 8.2, 1.6 Hz, 1H), 6.79 (d, J = 8.4 Hz, 1H), 6.61 (s, 1H), 6.52 (d, J = 2.2 Hz, 1H), 6.38 (dd, J = 8.2, 2.2 Hz, 1H), 3.84 (t, J = 5.0 Hz, 4H), 3.80 (s, 3H), 3.74 (s, 3H), 3.19 (p, J = 3.4 Hz, 4H). | 435.2 |
| 97 | 4-(6-(4-aminopiperidin-1- yl)-3-(4-hydroxy-4- methoxyphenyl)-4- methoxypyridin-2-yl)-2- fluorobenzonitrile | 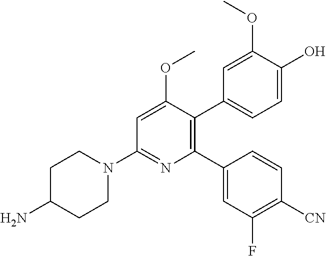 [SMILES: COc1cc(-c2c(OC)cc(N3CCC(N)CC3)nc2-c2ccc(C#N)c(F)c2)ccc1O] | ¹H NMR (400 MHz, DMSO-d₆) δ ppm 9.4-8.6(m, 1H), 7.72 (dd, J = 8.2, 7.0 Hz, 1H), 7.31-7.12 (m, 2H), 6.64 (d, J = 8.0 Hz, 1H), 6.56 (d, J = 1.8 Hz, 1H), 6.48 (s, 1H), 6.42 (dd, J = 8.2, 2.0 Hz, 1H), 4.28 (dt, J = 13.0, 3.8 Hz, 2H), 3.78 (s, 3H), 3.57 (s, 3H), 2.92 (ddd, J = 13.6, 11.4, 2.8 Hz, 2H), 2.85-2.75 (m, 1H), 1.78 (dd, J = 13.2, 3.6 Hz, 2H), 1.29-1.16 (m, 2H) | 449.2 |
| 98 | 4-(6-(4-aminopiperidin-1- yl)-3-(4-chloro-3- hydroxyphenyl)-4- methoxypyridin-2-yl)-2- fluorobenzonitrile hydrochloride | 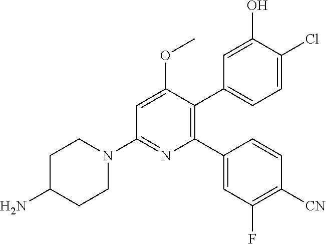 [SMILES: COc1cc(N2CCC(N)CC2)nc(-c2ccc(C#N)c(F)c2)c1-c1ccc(Cl)c(O)c1] | ¹H NMR (400 MHz, DMSO-d₆) δ ppm 10.11 (s, 1H), 8.18 (d, J = 5.2 Hz, 3H), 7.77 (dd, J = 8.2, 6.8 Hz, 1H), 7.37 (dd, J = 10.6, 1.4 Hz, 1H), 7.23 − 7.09 (m, 2H), 6.70 (d, J = 2.0 Hz, 1H), 6.59 (s, 1H), 6.46 (dd, J = 8.2, 2.0 Hz, 1H), 4.46 (d, J = 13.6 Hz, 2H), 3.82 (s, 3H), 3.32 (d, J = 5.0 Hz, 1H) 2.99 (t, J = 12.6 Hz, 2H), 1.99 (dd, J = 12.8, 3.8 Hz, 2H), 1.56 (qd, J = 12.2, 3.8 Hz, 2H). | 453.1 |
| 99 | 4-(6-(4-aminopiperidin-1- yl)-3-(3-hydroxy-4- (trifluoromethyl)phenyl)- 4-methoxypyridin-2-yl)- 2-fluorobenzonitrile hydrochloride | 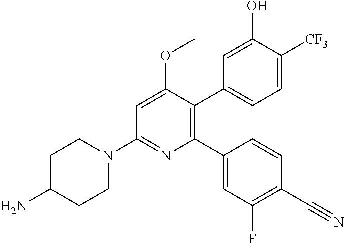 [SMILES: COc1cc(N2CCC(N)CC2)nc(-c2ccc(C#N)c(F)c2)c1-c1ccc(C(F)(F)F)c(O)c1] | ¹H NMR (400 MHz, Methanol-d₄) δ ppm: 7.80 − 7.70 (m, 1H), 7.56 (d, J = 9.8 Hz, 1H), 7.41 (d, J = 8.0 Hz, 1H), 7.37 − 7.32 (m, 1H), 6.83 (s, 1H), 6.73 (s, 1H), 6.69 (d, J = 8.2 Hz, 1H), 4.42 (d, J = 13.8 Hz, 2H), 4.06 (s, 3H), 3.56 (td, J = 11.2, 5.4 Hz, 1H), 3.49 − 3.36 (m, 2H), 2.32 − 2.14 (m, 2H), 1.89 (qd, J = 14.0, 13.2, 8.2 Hz, 2H). | 487.2 |
| 100 | 2-fluoro-4-(3-(3-hydroxy- 4-methylphenyl)-4- methoxy-6-(4- methylpiperazin- 1-yl)pyridin-2-yl) benzonitrile | 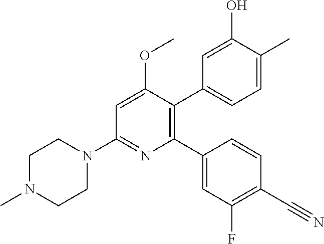 [SMILES: COc1cc(N2CCN(C)CC2)nc(-c2ccc(C#N)c(F)c2)c1-c1ccc(C)c(O)c1] | ¹H NMR (400 MHz, DMSO-d₆) δ ppm 9.11 (s, 1H), 7.71 (t, J = 7.6 Hz, 1H), 7.35 − 7.25 (m, 1H), 7.18 (dd, J = 8.0, 1.6 Hz, 1H), 6.92 (d, J = 7.6 Hz, 1H), 6.49 (s, 2H), 6.35 (dd, J = 7.6, 1.6 Hz, 1H), 3.77 (s, 3H), 3.57 (t, J = 5.0 Hz, 4H), 2.42 (t, J = 5.2 Hz, 4H), 2.23 (s, 3H), 2.08 (s, 3H). | 433.2 |

### Example 101

### Preparation of 4-(6-(4-aminopiperidin-1-yl)-3-(3-hydroxy-4-methoxyphenyl)-4-methoxy-5-methylpyridin-2-yl)-2-fluorobenzonitrile

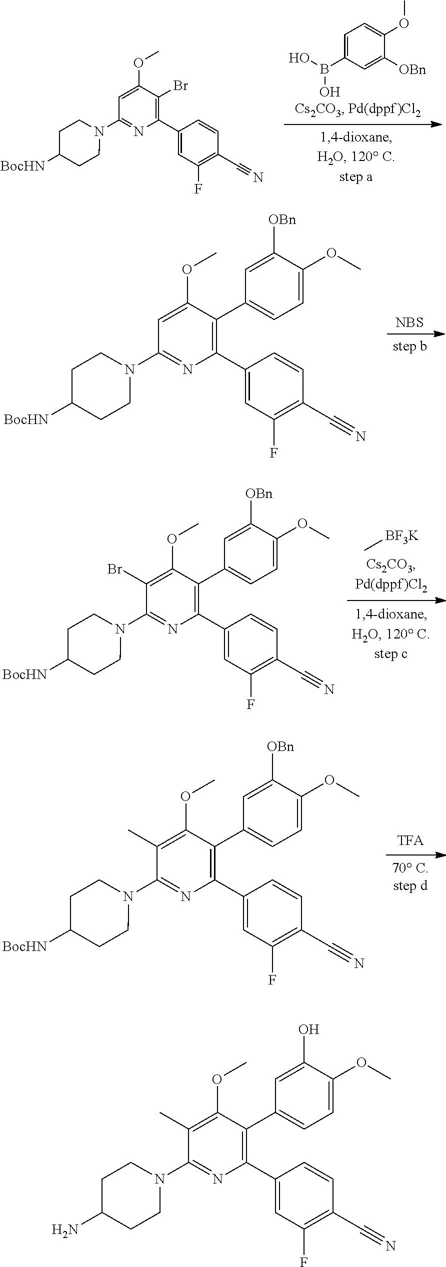

### Step a): Preparation of tert-butyl(1-(5-(3-(benzyloxy)-4-methoxyphenyl)-6-(4-cyano-3-fluorophenyl)-4-methoxypyridin-2-yl)piperidin-4-yl)carbamate

Tert-butyl(1-(5-bromo-6-(4-cyano-3-fluorophenyl)-4-methoxypyridin-2-yl)piperidin-4-yl)carbam ate (400 mg, 0.794 mmol), (3-(benzyloxy)-4-methoxyphenyl)boronic acid (307 mg, 1.191 mmol), Cs₂CO₃ (518 mg, 1.588 mmol), Pd(dppf)Cl₂ (58 mg, 0.079 mmol), 1.4-Dioxane (12 mL) and H₂O (3 mL) are added to a reaction flask, and reacted at 120° C. for 1 h while stirring. The reaction solution is concentrated under a reduced pressure to dryness; and the residue is purified by silica gel chromatography (eluent: petroleum ether/ethyl acetate=1/1) to obtain tert-butyl(1-(5-(3-(benzyloxy)-4-methoxyphenyl)-6-(4-cyano-3-fluorophenyl)-4-methoxypyridin-2-yl)piperidin-4-yl)carbamate with a yield of 48.0%.

ESI-MS(m/z)=639.3[M+H]⁺.

### Step b): Preparation of tert-butyl(1-(5-(3-(benzyloxy)-4-methoxyphenyl)-3-bromo-6-(4-cyano-3-fluorophenyl)-4-methoxypyridin-2-yl)piperidin-4-yl)carbamate

Tert-butyl(1-(5-(3-(benzyloxy)-4-methoxyphenyl)-6-(4-cyano-3-fluorophenyl)-4-methoxypyridin-2-yl)piperidin-4-yl)carbamate (244 mg, 0.381 mmol) and DMF (20 mL) are added to a reaction flask, added with NBS in batches (102 mg, 0.572 mmol) under ice bath agitation, and reacted at room temperature for 30 min while stirring. When the reaction is completed, the reaction solution is added with water (40 mL), and extracted with ethyl acetate (40 mL×3); organic phase is combined, and washed with saturated saline (40 mL×2), and concentrated under a reduced pressure to dryness; and the residue is purified by silica gel chromatography (eluent: petroleum ether/ethyl acetate=1/1) to obtain tert-butyl (1-(5-(3-(benzyloxy)-4-methoxyphenyl)-3-bromo-6-(4-cyano-3-fluorophenyl)-4-methoxypyridin-2-yl)piperidin-4-yl)carbamate with a yield of 72.0%.

¹H NMR (400 MHz, DMSO-d₆) δppm 7.71 (dt, J=7.8, 6.2 Hz, 1H), 7.39-7.25 (m, 6H), 7.16-7.05 (m, 1H), 6.98-6.84 (m, 2H), 6.68 (dd, J=8.4, 2.0 Hz, 1H), 4.99 (s, 2H), 3.78 (s, 3H), 3.38 (s, 3H), 3.31 (s, 2H), 2.87 (t, J=11.8 Hz, 2H), 1.99 (s, 1H), 1.84 (d, J=12.0 Hz, 2H), 1.42 (d, J=17.0 Hz, 9H).

ESI-MS(m/z)=717.2[M+H]⁺.

### Step c): Preparation of tert-butyl (1-(5-(3-(benzyloxy)-4-methoxyphenyl)-6-(4-cyano-3-fluorophenyl)-4-methoxy-3-methylpyridin-2-yl)piperidin-4-yl)carbamate

### Tert-butyl

(1-(5-(3-(benzyloxy)-4-methoxyphenyl)-3-bromo-6-(4-cyano-3-fluorophenyl)-4-methoxypyridin-2-yl)piperidin-4-yl)carbamate (100 mg, 0.140 mmol), potassium methyltrifluoroborate (34 mg, 0.280 mmol), Cs₂CO₃ (95 mg, 0.290 mmol), Pd(dppf)Cl₂ (11 mg, 0.015 mmol), 1.4-dioxane (4 mL) and H₂O (1 mL) are added to a reaction flask, and reacted at 120° C. for 1 h while stirring. The reaction solution is concentrated under a reduced pressure to dryness; and the residue is purified by silica gel chromatography (eluent: petroleum ether/ethyl acetate=1/1) to obtain tert-butyl (1-(5-(3-(benzyloxy)-4-methoxyphenyl)-6-(4-cyano-3-fluorophenyl)-4-methoxy-3-methylpyridin-2-yl)piperidin-4-yl)carbamate with a yield of 25.0%.

ESI-MS(m/z)=653.1[M+H]⁺.

### Step d): Preparation of 4-(6-(4-aminopiperidin-1-yl)-3-(3-hydroxy-4-methoxyphenyl)-4-methoxy-5-methylpyridin-2-yl)-2-fluorobenzonitrile

### Tert-butyl

(1-(5-(3-(benzyloxy)-4-methoxyphenyl)-6-(4-cyano-3-fluorophenyl)-4-methoxy-3-methylpyridin-2-yl)piperidin-4-yl)carbamate (23 mg, 0.035 mmol) and TFA (3 mL) are added to a reaction flask, stirred for dissolving, stirred at 70° C. for 16 h; a large amount of solids precipitate, which are concentrated under a reduced pressure to obtain a crude product; and the crude product is purified by Prep-HPLC (separation method 4) to obtain 4-(6-(4-aminopiperidin-1-yl)-3-(3-hydroxy-4-methoxyphenyl)-4-methoxy-5-methylpyridin-2-yl)-2-fluorobenzonitrile with a yield of 62.5%.

¹H NMR (400 MHz, DMSO-d₆) δppm 7.86-7.63 (m, 1H), 7.30 (dd, J=11.0, 1.8 Hz, 1H), 7.19 (dt, J=8.0, 1.8 Hz, 1H), 6.86 (d, J=8.4 Hz, 1H), 6.58 (d, J=2.0 Hz, 1H), 6.50 (dd, J=8.2, 2.0 Hz, 1H), 3.76 (s, 3H), 3.48 (d, J=12.4 Hz, 2H), 3.39 (s, 3H), 2.79 (p, J=13.4, 12.4 Hz, 3H), 2.20 (s, 3H), 1.83 (d, J=12.4 Hz, 2H), 1.50 (dt, J=57.4, 11.4 Hz, 2H).

ESI-MS(m/z)=463.5 [M+H]⁺.

### Example 102

### Preparation of 4-(6-(4-aminopiperidin-1-yl)-4-(3-fluoro-4-methoxyphenyl)-3-methoxypyridin-2-yl)-2-fluoroben zonitrile

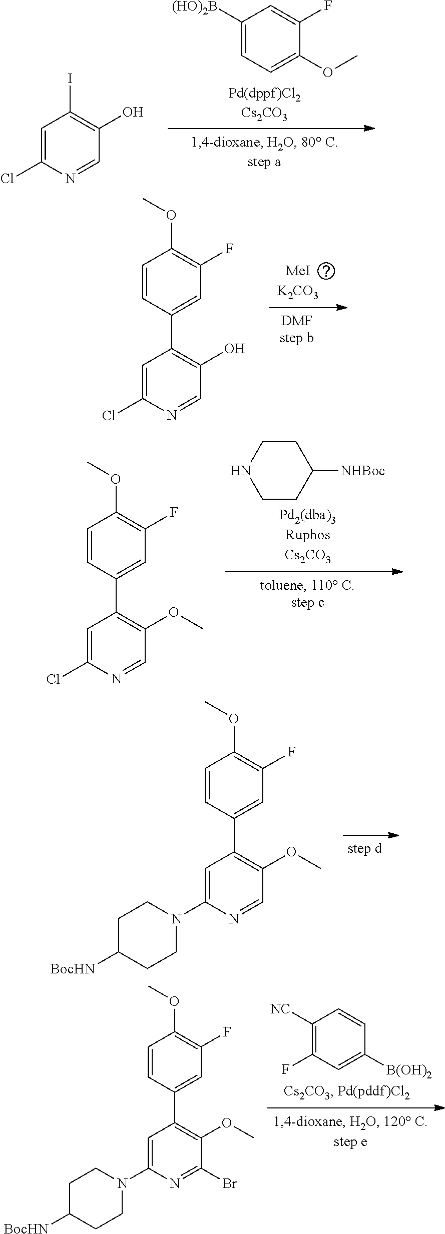

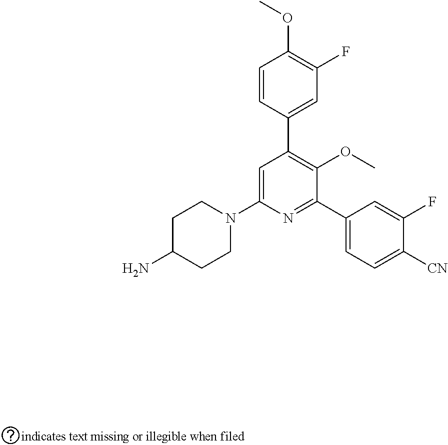 [SMILES: COc1ccc(-c2cc(N3CCC(N)CC3)nc(-c3ccc(C#N)c(F)c3)c2OC)cc1F]

### Step a): Preparation of 6-chloro-4-(3-fluoro-4-methoxyphenyl)pyridine-3-ol

6-chloro-4-iodopyridin-3-ol (500 mg, 1.961 mmol), (3-fluoro-4-methoxyphenyl)boronic acid (400 mg, 2.353 mmol), Pd(dppf)Cl₂ (143 mg, 0.196 mmol), Cs₂CO₃ (1.3 g, 3.922 mmol), 1,4-dioxane (12 mL) and water (3 mL) are sequentially added to a microwave reactor, subjected to nitrogen displacement for three times, warmed to 80° C., and reacted for 1 min while stirring. When the reaction is completed, the reaction solution is concentrated under a reduced pressure; the residue is purified by silica gel chromatography (eluent: petroleum ether/ethyl acetate=2/1) to obtain 6-chloro-4-(3-fluoro-4-methoxyphenyl)pyridine-3-ol with a yield of 80.0%.

ESI-MS(m/z)=253.2[M+H]⁺.

### Step b): Preparation of 2-chloro-4-(3-fluoro-4-methoxyphenyl)-5-methoxypyridine

6-chloro-4-(3-fluoro-4-methoxyphenyl)pyridine-3-ol (397 mg, 1.569 mmol), iodomethane (267 mg, 1.883 mmol), K₂CO₃ (433 mg, 3.138 mmol), and DMF (5 mL) are sequentially added to a reaction flask, subjected to nitrogen displacement for three times, and reacted at 80° C. for 2 h while stirring. When the reaction is completed, the reaction solution is added with water (50 mL), and extracted with ethyl acetate (50 mL×2); organic phase is combined, washed with saturated saline (50 mL×2), dried with anhydroous sodium sulfate, and filtered to obtain filtrate; the filtrate is concentrated under a reduced pressure; and the residue is purified by silica gel chromatography (eluent: petroleum ether/ethyl acetate=2/1) to obtain 2-chloro-4-(3-fluoro-4-methoxyphenyl)-5-methoxypyridine with a yield of 71.4%.

ESI-MS(m/z)=268.0[M+H]⁺.

### Step c): Preparation of tert-butyl (1-(4-(3-fluoro-4-methoxyphenyl)-5-methoxypyridin-2-yl)piperidin-4-yl)carbamate

2-Chloro-4-(3-fluoro-4-methoxyphenyl)-5-methoxypyridine (300 mg, 1.120 mmol), tert-butyl piperidin-4-yl-carbamate (448 mg, 2.240 mmol), Ruphos (10 mg, 0.011 mmol), Cs₂CO₃ (730 mg, 2.240 mmol), toluene (10 mL) and Pd(dba)₂ (19 mg, 0.033 mmol) are sequentially added to a reaction flask, subjected to nitrogen displacement for three times, warmed to 100° C. and reacted for 3 h while stirring. When the reaction is completed, the reaction is quenched by adding water (10 mL), and extracted with ethyl acetate (40 mL×2); organic phase is combined, and washed with saturated saline (30 mL×1), and concentrated under a reduced pressure; and the residue is purified by silica gel chromatography (eluent: petroleum ether/ethyl acetate=2/1) to obtain tert-butyl (1-(4-(3-fluoro-4-methoxyphenyl)-5-methoxypyridin-2-yl)piperidin-4-yl)carbamate with a yield of 28.4%.

¹H NMR (400 MHz, DMSO-d₆) δppm 7.93 (s, 1H), 7.53-7.37 (m, 2H), 7.23 (t, J=8.8 Hz, 1H), 6.84 (s, 1H), 4.23-4.06 (m, 2H), 3.88 (s, 3H), 3.74 (s, 3H), 3.45 (s, 3H), 2.86 (t, J=12.2 Hz, 2H), 1.77 (d, J=11.8 Hz, 2H), 1.38 (s, 9H).

ESI-MS m/z)=432.3 [M+H]⁺.

### Step d): Preparation of tert-butyl (1-(6-bromo-4-(3-fluoro-4-methoxyphenyl)-5-methoxypyridin-2-yl)piperidin-4-yl)carbamate

Tert-butyl (1-(4-(3-fluoro-4-methoxyphenyl)-5-methoxypyridin-2-yl)piperidin-4-yl)carbamate (162 mg, 0.318 mmol) and TfOH (5 mL) are added to a reaction flask, added with NBS in batches (68 mg,0.381 mmol) under ice bath agitation, and reacted while stirring in the ice bath at room temperature for 30 min. When the reaction is completed, the reaction solution is added into a sodium bicarbonate aqueous solution (40 mL), and extracted with ethyl acetate (40 mL×3); organic phase is combined, and washed with saturated saline (40 mL×2), and concentrated under a reduced pressure to dryness; and the residue is purified by silica gel chromatography (eluent: petroleum ether/ethyl acetate=3/2) to obtain tert-butyl (1-(6-bromo-4-(3-fluoro-4-methoxyphenyl)-5-methoxypyridin-2-yl)piperidin-4-yl)carbamate with a yield of 70.0%.

ESI-MS(m/z)=510.2[M+H]⁺.

### Step e): Preparation of 4-(6-(4-aminopiperidin-1-yl)-4-(3-fluoro-4-methoxyphenyl)-3-methoxypyridin-2-yl)-2-fluoroben zonitrile

### Tert-butyl

(1-(6-bromo-4-(3-fluoro-4-methoxyphenyl)-5-methoxypyridin-2-yl)piperidin-4-yl)carbamate (114 mg, 0.223 mmol), (3-fluoro-4-cyanophenyl)boronic acid (74 mg, 0.446 mmol), Pd(dppf)Cl₂ (17 mg, 0.023 mmol), Cs₂CO₃ (145 mg, 0.446 mmol), 1,4-dioxane (4 mL) and water (1 mL) are sequentially added to a microwave reactor, subjected to nitrogen displacement for three times, warmed to 120° C., and reacted for 30 min while stirring. When the reaction is ended, the reaction solution is concentrated under a reduced pressure; and the crude product is purified by Prep-HPLC (separation method 4) to obtain 4-(6-(4-aminopiperidin-1-yl)-4-(3-fluoro-4-methoxyphenyl)-3-methoxypyridin-2-yl)-2-fluoroben zonitrile with a yield of 34.0%.

¹H NMR (400 MHz, Methanol-d₄) δppm 7.95 (s, 1H), 7.85 (t, J=7.4 Hz, 1H), 7.68-7.47 (m, 3H), 7.42 (t, J=1.8 Hz, 1H), 6.89 (s, 1H), 4.19 (d, J=13.2 Hz, 2H), 3.95-3.74 (m, 6H), 3.09-2.82 (m, 3H), 2.09-1.96 (m, 2H), 1.51 (t, J=10.4 Hz, 2H).

ESI-MS(m/z)=451.2[M+H]⁺.

### Example 103

### Preparation of 4-(6-(4-aminopiperidin-1-yl)-4-ethoxy-3-(3-hydroxy-4-methoxyphenyl)pyridin-2-yl)-2-fluorobe nzonitrile

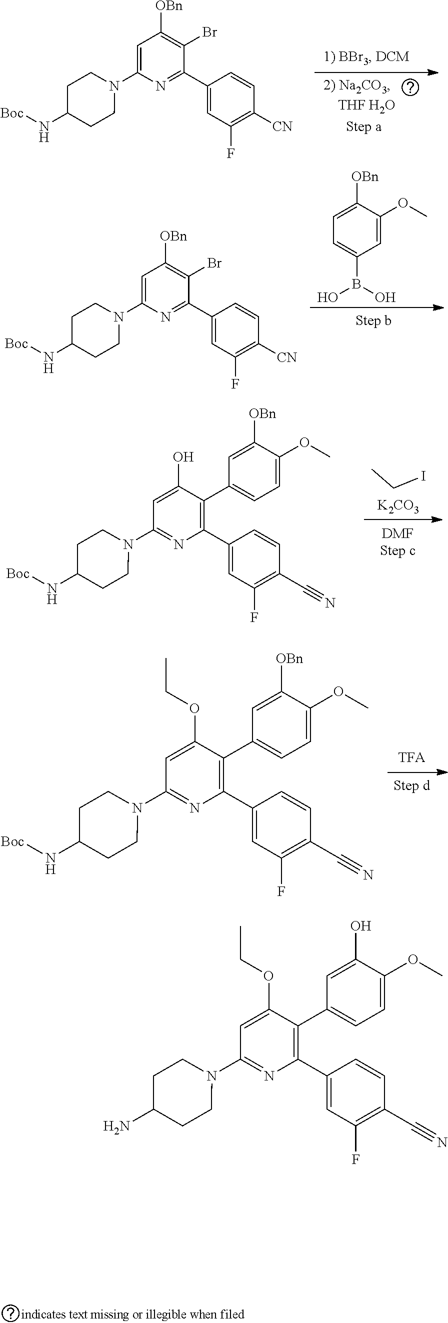

### Step a): Preparation of tert-butyl (1-(5-bromo-6-(4-cyano-3-fluorophenyl)-4-hydroxypyridin-2-yl)piperidin-4-yl)carbamate

### Tert-butyl

1-(4-(benzyloxy)-5-bromo-6-(4-cyano-3-fluorophenyl)pyridin-2-yl)piperidin-4-yl)carbamate is dissolved in 20 ml of dichloromethane (3 g, 5.1 mmol), stirred at −78° C. for 15 min, added dropwise with 20 mL of 1M boron tribromide solution in hexane and reacted for 1 h; detected until the reaction is completed, the reaction solution is quenched with a saturated sodium bicarbonate aqueous solution, and an organic solvent is dried by spinning; sodium carbonate is added to adjust pH=8-9 in an ice bath; the reaction solution is added with di-tert-butyl dicarbonate, reacted for 30 min, and quenched with water (200 mL); the reaction solution is extracted with ethyl acetate (200 mL×2); organic phase is combined, and washed with saturated saline (150 mL×2), dried with anhydrous sodium sulfate, filtered and concentrated to obtain a crude product; and the residue is purified by silica gel chromatography (eluent: petroleum ether/ethyl acetate=10/3) to obtain tert-butyl(1-(5-(3-(benzyl)-4-methoxyphenyl)-6-(4-cyano-3-fluorophenyl)-4-hydroxypyridin-2-y 1)piperidin-4-yl)carbamate with a yield of 70.2%.

¹H NMR (400 MHz, DMSO-d₆) δ ppm 11.62 (s, 1H), 8.10-7.93 (m, 1H), 7.74 (d, J=10.2 Hz, 1H), 7.65 (d, J=8.0 Hz, 1H), 7.05 (d, J=1.6 Hz, 1H), 6.85 (d, J=7.8 Hz, 1H), 4.23 (d, J=13.4 Hz, 2H), 3.54 (d, J=10.6 Hz, 1H), 2.98 (t, J=12.4 Hz, 2H), 1.89-1.74 (m, 2H), 1.39 (d, J=1.8 Hz, 11H).

ESI-MS m/z=491.1[M+H]⁺.

### Step b): Preparation of tert-butyl(1-(5-(3-(benzyl)-4-methoxyphenyl)-6-(4-cyano-3-fluorophenyl)-4-hydroxypyridin-2-y 1)piperidin-4-yl)carbamate

### Tert-butyl

1-(5-bromo-6-(4-cyano-3-fluorophenyl)-4-hydroxypyridin-2-yl)piperidin-4-yl)carbamate (400 mg, 0.16 mmol), (4-(benzyloxy)-3-methoxyphenyl)boronic acid (237 mg, 0.25 mmol), Cs₂CO₃ (532 mg, 1.6 mmol), and Pd(dppf)Cl₂ (60 mg, 0.08 mmol) are dissolved in 10 mL of dioxane and added with 1 mL of water; and the mixed reaction solution is purged by nitrogen blowing and protected with nitrogen. The mixture reacts in a microwave reactor at 120° C. for 30 min; when LC-MS shows that the reaction is completed, a crude product is obtained by concentration, and the residue is purified by silica gel chromatography (eluent: petroleum ether/ethyl acetate=10/3) to obtain tert-butyl(1-(5-(3-(benzyloxy)-4-methoxyphenyl)-3-bromo-6-(4-cyano-3-fluorophenyl)-4-methoxypyridin-2-yl)piperidin-4-yl)carbamate with a yield of 63.4%.

ESI-MS m/z=625.3 [M+H]⁺.

### Step c): Preparation of tert-butyl(1-(5-(3-(benzyloxy)-4-methoxyphenyl)-6-(4-cyano-3-fluorophenyl)-4-ethoxypyridin-2-yl)piperidin-4-yl)carbamate

### Tert-butyl

(1-(5-(3-(benzyloxy)-4-methoxyphenyl)-6-(4-cyano-3-fluorophenyl)-4-hydroxypyridin-2-yl)pipe ridin-4-yl)carbamate (50 mg, 0.08 mmol), iodoethane (37 mg, 0.24 mmol), and K₂CO₃ (33 mg, 0.24 mmol) are dissolved in 5 mL of N,N-dimethylformamide; and the mixed reaction solution is purged by nitrogen blowing and protected with nitrogen. The reaction solution reacts at 60° C. for 2 h; when LC-MS shows that the reaction is completed, a crude product is obtained by concentration, and delivered to Prep-HPLC preparation (separation method 4) to obtain tert-butyl(1-(5-(3-(benzyloxy)-4-methoxyphenyl)-6-(4-cyano-3-fluorophenyl)-4-ethoxypyridin-2-yl)piperidin-4-yl)carbamate with a yield of 43.6%.

ESI-MS m/z=653.3 [M+H]⁺.

### Step d): Preparation of 4-(6-(4-aminopiperidin-1-yl)-4-ethoxy-3-(3-hydroxy-4-methoxyphenyl)pyridin-2-yl)-2-fluorobe nzonitrile

### Tert-butyl

(1-(5-(3-(benzyloxy)-4-methoxyphenyl)-6-(4-cyano-3-fluorophenyl)-4-ethoxypyridin-2-yl)piperi din-4-yl)carbamate (24 mg, 0.034 mmol) is added to 4 mL of trifluoroacetic acid solution, and reacted at 70° C. for 2 h; when LC-MS shows that the reaction is completed, the solvent is dried by spinning at low temperature to dryness; and the crude product is subjected to Prep-HPLC preparation (separation method 4) to obtain 4-(6-(4-aminopiperidin-1-yl)-4-ethoxy-3-(3-hydroxy-4-methoxyphenyl)pyridin-2-yl)-2-fluorobe nzonitrile with a yield of 41.5%.

¹H NMR (400 MHz, Methanol-d₄) δ ppm 7.51 (dd, J=8.0, 6.8 Hz, 1H), 7.29-7.20 (m, 2H), 6.81 (d, J=8.4 Hz, 1H), 6.59 (d, J=2.2 Hz, 1H), 6.54-6.38 (m, 2H), 4.42 (s, 2H), 4.10 (t, J=7.0 Hz, 2H), 3.85 (s, 3H), 3.03-2.81 (m, 3H), 1.96 (d, J=12.2 Hz, 2H), 1.47 (dd, J=11.8, 4.0 Hz, 2H), 1.32 (t, J=7.0 Hz, 3H).

ESI-MS m/z=463.2 [M+H]⁺.

### Example 104

### 4-(6-(4-aminopiperidin-1-yl)-3-(3-hydroxy-4-methoxyphenyl)-4-isopropoxypyridin-2-yl)-2-fluor obenzonitrile is prepared according to the synthesis method (separation method 4) of Example 103, and the structure and representation data are as follows

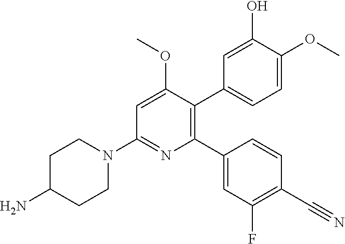 [SMILES: COc1ccc(-c2c(OC)cc(N3CCC(N)CC3)nc2-c2ccc(C#N)c(F)c2)cc1O]

¹H NMR (400 MHz, Methanol-d₄) δ ppm 7.39 (dd, J=8.0, 6.8 Hz, 1H), 7.18-7.02 (m, 2H), 6.68 (d, J=8.4 Hz, 1H), 6.46 (d, J=2.0 Hz, 1H), 6.39-6.28 (m, 2H), 4.60 (d, J=6.2 Hz, 1H), 4.29 (d, J=13.2 Hz, 2H), 3.73 (s, 3H), 2.93-2.76 (m, 3H), 1.90-1.76 (m, 2H), 1.34 (qd, J=12.2, 4.2 Hz, 2H), 1.16 (d, J=6.0 Hz, 6H).

ESI-MS m/z=477.2 [M+H]⁺.

### Example 105

### 4-(6-(4-aminopiperidin-1-yl)-4-(cyclopentyloxy)-3-(3-hydroxy-4-methoxyphenyl)pyridin-2-yl)-2-fluorobenzonitrile is prepared according to the synthesis method (separation method 4) of Example 103, and the structure and representation data are as follows

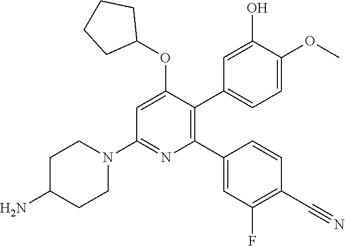 [SMILES: COc1ccc(-c2c(OC3CCCC3)cc(N3CCC(N)CC3)nc2-c2ccc(C#N)c(F)c2)cc1O]

¹H NMR (400 MHz, Methanol-d₄) δppm 7.51 (dd, J=8.0, 6.6 Hz, 1H), 7.34-7.17 (m, 2H), 6.80 (d, J=8.4 Hz, 1H), 6.57 (d, J=2.0 Hz, 1H), 6.47-6.37 (m, 2H), 4.94 (dq, J=5.8, 3.2, 2.8 Hz, 1H), 4.41 (dd, J=13.2, 3.4 Hz, 2H), 3.85 (s, 3H), 2.97 (td, J=13.0, 12.6, 2.6 Hz, 3H), 1.92 (dddd, J=19.2, 7.8, 5.4, 2.8 Hz, 4H), 1.76 (dd, J=14.0, 5.4 Hz, 2H), 1.63 (dtt, J=14.6, 7.8, 3.8 Hz, 4H), 1.45 (dd, J=11.4, 3.8 Hz, 2H).

ESI-MS m/z=503.2 [M+H]⁺.

### Example 106

### Preparation of 4-(6-(4-aminopiperidin-1-yl)-4-(difluoromethoxy)-3-(3-hydroxy-4-methoxyphenyl)pyridin-2-yl)-2-fluorobenzonitrile

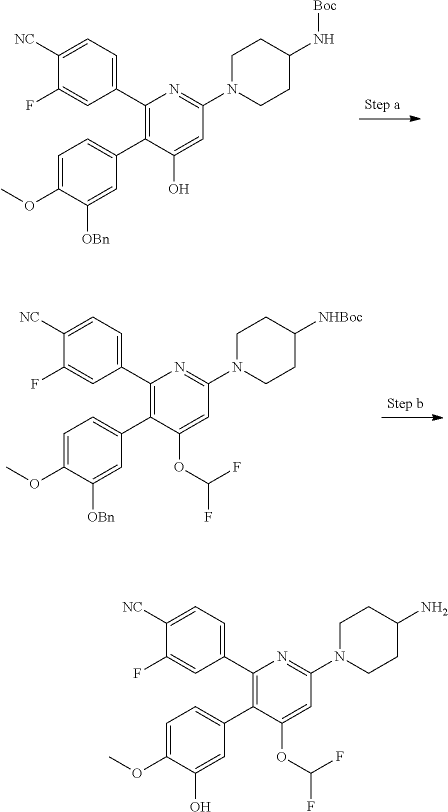 [SMILES: COc1ccc(-c2c(O)cc(N3CCC(NC(=O)OC(C)(C)C)CC3)nc2-c2ccc(C#N)c(F)c2)cc1OCc1ccccc1 ; COc1ccc(-c2c(OC(F)F)cc(N3CCC(NC(=O)OC(C)(C)C)CC3)nc2-c2ccc(C#N)c(F)c2)cc1OCc1ccccc1 ; COc1ccc(-c2c(OC(F)F)cc(N3CCC(N)CC3)nc2-c2ccc(C#N)c(F)c2)cc1O]

### Step a): Synthesis of tert-butyl(1-(5-(3-(benzyloxy)-4-methoxyphenyl)-6-(4-cyano-3-fluorophenyl)-4-(difluoromethox y)pyridin-2-yl)piperidin-4-yl)carbamate

Tert-butyl(1-(5-(3-(benzyloxy)-4-methoxyphenyl)-6-(4-cyano-3-fluorophenyl)-4-hydroxypyridin-2-yl)piperidin-4-yl)carbamate (75 mg, 0.12 mmol), potassium hydroxide (20.2 mg, 0.36 mmol), and diethyl(bromofluoromethyl)phosphonate (64 mg, 0.24 mmol) are dissolved in acetonitrile (3.2 mL) and water (0.2 mL), and reacted at room temperature for 16 h while stirring under nitrogen protection. When LC-MS shows that the reaction is completed, the reaction solution is added with water (10 mL), extracted with ethyl acetate (10 mL×3), washed with saturated saline (10 mL), dried with anhydrous sodium sulfate, filtered, and concentrated; and the residue is purified by silica gel chromatography (eluent: petroleum ether/ethyl acetate=3: 1) to obtain tert-butyl(1-(5-(3-(benzyloxy)-4-methoxyphenyl)-6-(4-cyano-3-fluorophenyl)-4-(difluoromethox y)pyridin-2-yl)piperidin-4-yl)carbamate with a yield of 69.1%.

¹HNMR (400 MHz, DMSO-d₆) δ ppm 7.69 (dd, J=8.2, 6.8 Hz, 1H), 7.43-7.19 (m, 7H), 7.09 (dd, J=8.2, 1.6 Hz, 1H), 6.88 (dd, J=20.4, 8.2 Hz, 2H), 6.76-6.67 (m, 2H), 6.62 (dd, J=8.2, 2.0 Hz, 1H), 5.75 (s, 1H), 4.91 (s, 2H), 4.29 (d, J=13.4 Hz, 2H), 3.76 (s, 3H), 3.54 (s, 1H), 2.98 (t, J=12.4 Hz, 3H), 1.81 (d, J=12.4 Hz, 2H), 1.39 (s, 9H).

ESI-MS m/z: 675.3[M+H]⁺.

### Step b): Preparation of 4-(6-(4-aminopiperidin-1-yl)-4-(difluoromethoxy)-3-(3-hydroxy-4-methoxyphenyl)pyridin-2-yl)-2-fluorobenzonitrile

Tert-butyl(1-(5-(3-(benzyloxy)-4-methoxyphenyl)-6-(4-cyano-3-fluorophenyl)-4-(difluorometho xy)pyridin-2-yl)piperidin-4-yl)carbamate (56 mg, 0.08 mmol) is added to trifluoroacetic acid (3 mL) in a sealed tube, and warmed to 70° C. and reacted for 2 h under nitrogen protection; when the reaction is completed, the system is concentrated to dryness to obtain a crude product; and the crude product is purified by a Prep-HPLC preparation column (separation method 4) and freeze-dried to obtain 4-(6-(4-aminopiperidin-1-yl)-4-(difluoromethoxy)-3-(3-hydroxy-4-methoxyphenyl)pyridin-2-yl)-2-fluorobenzonitrile with a yield of 19.6%.

¹HNMR (400 MHz, DMSO-d₆) δ ppm 8.92 (s, 1H), 7.76 (t, J=7.6 Hz, 1H), 7.30 (dd, J=10.8, 1.4 Hz, 1H), 7.25-7.17 (m, 1H), 6.82 (d, J=8.4 Hz, 1H), 6.68 (s, 1H), 6.52 (d, J=2.2 Hz, 1H), 6.43 (dd, J=8.2, 2.2 Hz, 1H), 4.25 (dt, J=13.4, 3.8 Hz, 2H), 3.75 (s, 3H), 3.04-2.92 (m, 2H), 2.84 (tt, J=10.2, 3.8 Hz, 1H), 1.79 (dd, J=13.2, 3.8 Hz, 2H), 1.30-1.17 (m, 2H).

ESI-MS m/z: 485.2[M+H]⁺.

Compounds in Examples 107-109 are prepared according to the synthesis method of Example 106 (separation method 1), and their structures and characterization data are as follows

| | | | | MS |
|---|---|---|---|---|
| Examples | Chemical name | Structure | ¹H NMR | (M + H)⁺ |
| 107 | 4-(6-(4- aminopiperidin- 1-yl)-4- (cyclobutylmethoxy)- 3-(3-hydroxy-4- methoxyphenyl) pyridin-2-yl)-2- fluorobenzonitrile hydrochloride | 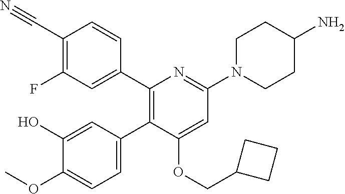 [SMILES: COc1ccc(-c2c(OCC3CCC3)cc(N3CCC(N)CC3)nc2-c2ccc(C#N)c(F)c2)cc1O] | ¹H NMR (400 MHz, DMSO-d₆ + D₂O) δ ppm 7.76 (t, J = 7.4 Hz, 1H), 7.35 (d, J = 10.0 Hz, 1H), 7.20 (dt, J = 8.2, 2.2 Hz, 1H), 6.78 (d, J = 8.4 Hz, 1H), 6.65 − 6.46 (m, 2H), 6.38 (dt, J = 8.2, 1.8 Hz, 1H), 4.43 (d, J = 13.4 Hz, 2H), 4.02 (d, J = 6.0 Hz, 2H), 3.73 (s, 3H), 3.31 (s, 1H), 2.97 (d, J = 11.8 Hz, 2H), 2.60 (p, J = 7.6, 7.0 Hz, 1H), 1.94 (dtd, J = 10.5, 5.4, 3.2 Hz, 4H), 1.83 − | 503.2 |
| | | | 1.69 (m, 4H), 1.54 (d, J = 10.6 | |
| | | | Hz, 2H). | |
| 108 | 4-(6-(4- aminopiperidin- 1-yl)-4- (cyclopropylmethoxy)- 3-(3-hydroxy- 4-methoxyphenyl) pyridin-2-yl)-2- fluorobenzonitrile hydrochloride | 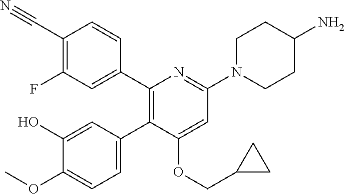 [SMILES: COc1ccc(-c2c(OCC3CC3)cc(N3CCC(N)CC3)nc2-c2ccc(C#N)c(F)c2)cc1O] | ¹H NMR (400 MHz, Methanol-d₄) δ ppm 7.71 (t, J = 7.0 Hz, 1H), 7.47 (d, J = 9.6 Hz, 1H), 7.31 (d, J = 7.8 Hz, 1H), 6.89 − 6.66 (m, 2H), 6.59 (s, 1H), 6.48 (d, J = 8.2 Hz, 1H), 4.31 (d, J = 12.8 Hz, 2H), 4.15 (d, J = 6.6 Hz, 2H), 3.81 (s, 3H), 3.63 − 3.47 (m, 1H), 3.38 (t, J = 12.2 Hz, 2H), 2.37 − 2.13 (m, 2H), 1.98 − 1.71 (m, 2H), 1.34 − 1.10 (m, 1H), 0.58 (dd, J = 7.8, 5.2 Hz, | 489.2 |
| | | | 2H), 0.33 (t, J = 4.8 Hz, 2H). | |
| 109 | 4-(6-(4-aminopiperidin- 1-yl)-3-(3-hydroxy-4- methoxyphenyl)-4- (oxetane-3-yl- methoxy)pyridin- 2-yl)-2- fluorobenzonitrile hydrochloride | 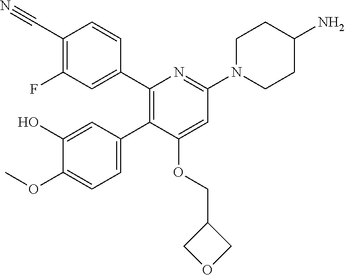 [SMILES: COc1ccc(-c2c(OCC3COC3)cc(N3CCC(N)CC3)nc2-c2ccc(C#N)c(F)c2)cc1O] | ¹H NMR (400 MHz, Methanol-d₄) δ ppm 7.69 (t, J = 6.8 Hz, 1H), 7.47 (d, J = 9.4 Hz, 1H), 7.32 (d, J = 7.8 Hz, 1H), 6.95 − 6.69 (m, 2H), 6.57 (s, 1H), 6.48 (d, J = 8.0 Hz, 1H), 4.35 (q, J = 12.8, 10.8 Hz, 4H), 3.78 (s, 3H), 3.67 − 3.30 (m, 7H), 2.22 (t, J = 11.8 Hz, 3H), 1.86 (t, J = 11.4 Hz, 2H). | 505.2 |

### Example 110

### Preparation of 2-(4-aminopiperidin-1-yl)-6-(4-cyano-3-fluorophenyl)-5-(3-fluoro-4-methoxyphenyl)pyrimidine-4-carboxamide hydrochloride

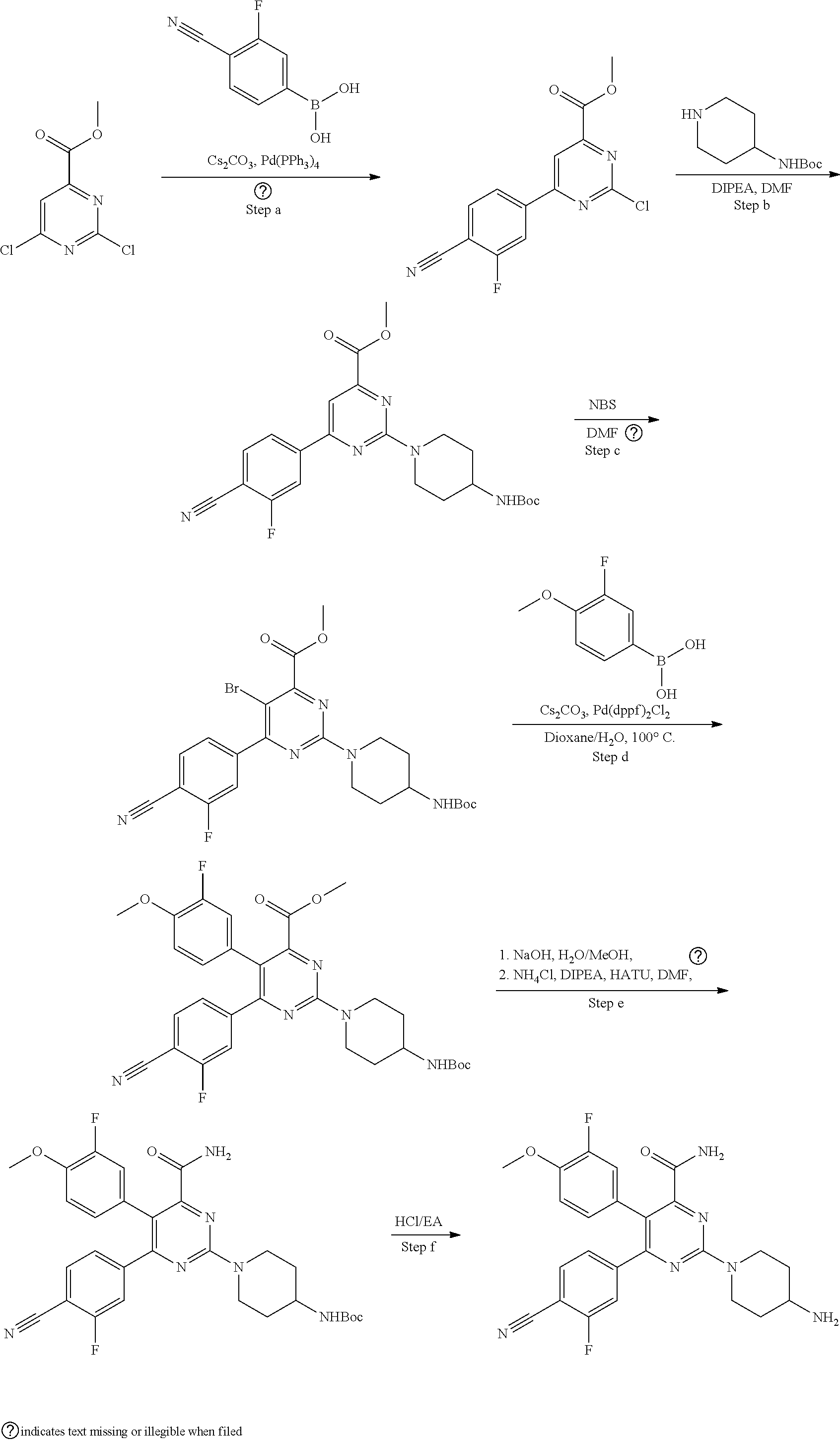

### Step a): Preparation of methyl 2-chloro-6-(4-cyano-3-fluorophenyl)pyrimidine-4-carboxylate

Methyl 2,6-dichloropyrimidine-4-carboxylate (2.1 g, 10.1 mmol), (4-cyano-3-fluorophenyl)boronic acid (1.67 g, 10.1 mmol), Cs₂CO₃ (6.6 g, 20.2 mmol), and Pd(dppf)Cl₂ (371 mg, 0.005 mmol) are dissolved in 20 mL of dioxane and 1 mL of water, and reacted in microwaves at 100° C. for 30 min. When LC-MS monitors that the raw materials are reacted completely, the reaction solution is cooled to room temperature, and concentrated under vacuum; and the residue is purified by silica gel chromatography (eluent: petroleum ether/ethyl acetate=10/3) to obtain methyl 2-chloro-6-(4-cyano-3-fluorophenyl)pyrimidine-4-carboxylate with a yield of 18.2%.

¹H NMR (400 MHz, DMSO-d₆) δ ppm 8.75 (d, J=1.8 Hz, 1H), 8.45 (d, J=10.6 Hz, 1H), 8.33 (d, J=8.4 Hz, 1H), 8.17 (t, J=7.8 Hz, 1H), 3.98 (d, J=1.8 Hz, 3H).

ESI-MS m/z=292.0[M+H]⁺.

### Step b): Preparation of methyl 2-(4-((tert-butoxycarbonyl)amino)piperidin-1-yl)-6-(4-cyano-3-fluorophenyl)pyrimidine-4-carbo xylate

Methyl 2-chloro-6-(4-cyano-3-fluorophenyl)pyrimidine-4-carboxylate (520 mg, 1.78 mmol), tert-butyl-piperidin-4-yl-carbamate (357.6 mg, 1.78 mmol), and DIPEA (460 mg, 3.56 mmol) are dissolved in DMF, and reacted for 2 h at room temperature, the reaction process is monitored by LC-MS. When LC-MS shows the reaction is completed, the reaction solution is quenched with 20 mL of water, and extracted with ethyl acetate (20 mL×2); organic phase is combined, washed with saturated saline (18 mL×2), dried with anhydrous sodium sulfate, and filtered; and the residue is purified by silica gel chromatography (eluent: dichloromethane/ethyl acetate=5/2) to obtain methyl 2-(4-((tert-butoxycarbonyl)amino)piperidin-1-yl)-6-(4-cyano-3-fluorophenyl)pyrimidine-4-carbo xylatewith a yield of 85.4%.

¹H NMR (400 MHz, DMSO-d₆) δ ppm 8.29 (d, J=10.8 Hz, 1H), 8.17 (d, J=8.4 Hz, 1H), 8.04 (t, J=7.6 Hz, 1H), 7.67 (s, 1H), 6.95 (d, J=7.4 Hz, 1H), 5.72 (t, J=2.0 Hz, 1H), 4.47 (d, J=12.8 Hz, 1H), 3.91 (d, J=2.6 Hz, 3H), 1.92-1.77 (m, 2H), 1.56-1.45 (m, 2H), 1.39 (s, 9H).

ESI-MS m/z=456.2[M+H]⁺.

### Step c): Preparation of methyl 5-bromo-2-(4-((tert-butoxycarbonyl)amino)piperidin-1-yl)-6-(4-cyano-3-fluorophenyl)pyrimidin e-4-carboxylate

### Methyl

2-(4-((tert-butoxycarbonyl)amino)piperidin-1-yl)-6-(4-cyano-3-fluorophenyl)pyrimidine-4-carbo xylate (750 mg, 1.64 mmol) and NBS (879.7 mg, 4.94 mmol) are dissolved in DMF, subjected to nitrogen displacement for three times, and reacted for 2 h in an ice bath, the reaction process is monitored by LC-MS. When LC-MS shows the reaction has been completed, the reaction solution is quenched with 20 mL of water, and extracted with ethyl acetate (20 mL×2); organic phase is combined, washed with saturated saline (15 mL×2), dried with anhydrous sodium sulfate, filtered; and the residue is purified by silica gel chromatography (eluent: dichloromethane/ethyl acetate=5/2) to obtain methyl 5-bromo-2-(4-((tert-butoxycarbonyl)amino)piperidin-1-yl)-6-(4-cyano-3-fluorophenyl)pyrimidin e-4-carboxylate with a yield of 91.3%.

ESI-MS m/z=534.1 [M+H]⁺.

### Step d): Preparation of methyl 2-(4-((tert-butoxycarbonyl)amino)piperidin-1-yl)-6-(4-cyano-3-fluorophenyl)-5-(3-fluoro-4-met hoxyphenyl)pyrimidine-4-carboxylate

### Methyl

5-bromo-2-(4-((tert-butoxycarbonyl)amino)piperidin-1-yl)-6-(4-cyano-3-fluorophenyl)pyrimidin e-4-carboxylate (430 mg, 0.80 mmol), (3-fluoro-4-methoxyphenyl) boronic acid (136 mg, 0.80 mmol), Cs₂CO₃ (524.2 mg, 1.60 mmol), and Pd(dppf)Cl₂ (58.8 mg, 0.080 mmol) are dissolved in 1,4-Dioxane/H₂O (10/1), subjected to nitrogen displacement for three times, and reacted in microwaves at 100° C. for 30 min, the reaction process is monitored by LC-MS. When LC-MS shows that the reaction has been completed, the reaction solution is cooled to room temperature, and then concentrated under vacuum; and the residue is purified by silica gel chromatography (eluent: petroleum ether/ethyl acetate=5/3) to obtain methyl 2-(4-((tert-butoxycarbonyl)amino)piperidin-1-yl)-6-(4-cyano-3-fluorophenyl)-5-(3-fluoro-4-met hoxyphenyl)pyrimidine-4-carboxylate with a yield of 71.4%.

¹H NMR (400 MHz, DMSO-d₆) δ ppm 7.84 (t, J=7.4 Hz, 1H), 7.60-7.49 (m, 1H), 7.22 (d, J=8.2 Hz, 1H), 7.09 (t, J=8.8 Hz, 1H), 6.98-6.94 (m, 1H), 6.89-6.79 (m, 1H), 4.43 (d, J=12.8 Hz, 1H), 3.82 (s, 3H), 3.62 (s, 3H), 3.57 (s, 1H), 3.45-3.36 (m, 1H), 3.11 (s, 2H), 1.91-1.86 (m, 1H), 1.81-1.74 (m, 1H), 1.51-1.44 (m, 2H), 1.38 (d, J=2.4 Hz, 9H).

ESI-MS m/z=580.2 [M+H]⁺.

### Step e): Preparation of tert-butyl(1-(4-carbamoyl-6-(4-cyano-3-fluorophenyl)-5-(3-fluoro-4-methoxyphenyl)pyrimidin-2-yl)piperidin-4-yl]carbamate

### Methyl

2-(4-((tert-butoxycarbonyl)amino)piperidin-1-yl)-6-(4-cyano-3-fluorophenyl)-5-(3-fluoro-4-met hoxyphenyl)pyrimidine-4-carboxylate (50 mg, 0.086 mmol) and NaOH (21 mg, 0.172 mmol) are dissolved in MeOH/H₂O, and reacted at room temperature for 2 h, the reaction process is monitored by LC-MS. When LC-MS shows that raw materials is reacted completely, 1N hydrochloric acid is prepared to adjust the reaction solution to PH=5, then added with ethyl acetate, and repeatedly extracted with saturated saline for three times; the organic phase is dried, and concentrated under vacuum to obtain 60 mg of crude product; the crude product is then dissolved in DMF, added with DIPEA (41 mg, 0.318 mmol), NH₄Cl (22.6 mg, 0.424 mmol), and HATU (60.5 mg, 0.159 mmol), and continues to react at room temperature for 1 h, the reaction process is monitored by LC-MS; when LC-MS shows that the reaction is completed, the reaction solution is concentrated under vacuum; and the residue is purified by silica gel chromatography (eluent: dichloromethane/ethyl acetate=5/2) to obtain tert-butyl (1-(4-carbamoyl-6-(4-cyano-3-fluorophenyl)-5-(3-fluoro-4-methoxyphenyl)pyrimidin-2-yl)piper idin-4-yl]carbamate with a yield of 67.3%.

¹H NMR (400 MHz, DMSO-d₆) δ ppm: 7.95 (s, 1H), 7.79 (t, J=7.4 Hz, 1H), 7.52 (s, 1H), 7.19 (d, J=8.2 Hz, 1H), 7.07-6.91 (m, 3H), 6.84 (d, J=8.4 Hz, 1H), 4.46 (d, J=35.0 Hz, 2H), 3.81 (s, 3H), 3.02 (d, J=41.2 Hz, 2H), 1.88 (s, 1H), 1.79 (s, 1H), 1.47 (d, J=9.0 Hz, 2H), 1.38 (s, 9H), 1.24 (d, J=9.2 Hz, 1H).

ESI-MS m/z=565.2[M+H]⁺.

### Step f): Preparation of 2-(4-aminopiperidin-1-yl)-6-(4-cyano-3-fluorophenyl)-5-(3-fluoro-4-methoxyphenyl)pyrimidin-4-carboxamide hydrochloride

### Tert-butyl

[1-(4-carbamoyl-6-(4-cyano-3-fluorophenyl)-5-(3-fluoro-4-methoxyphenyl)pyrimidin-2-yl)piper idin-4-yl]carbamate (40 mg, 0.07 mmol) is dissolved in 4M HCl (ethyl acetate solution) and reacted at room temperature for 0.5 h, the reaction process is monitored by LC-MS. When LC-MS shows that the reaction is completed, the reaction solution is concentrated under vacuum; and the solid is delivered to Prep-HPLC preparation (separation method 1) to obtain 2-(4-aminopiperidin-1-yl)-6-(4-cyano-3-fluorophenyl)-5-(3-fluoro-4-methoxyphenyl)pyrimidin-4-carboxamide hydrochloride with a yield of 91.2.

¹H NMR (400 MHz, DMSO-d₆) δ ppm 8.20 (s, 2H), 7.93 (s, 1H), 7.84 (dd, J=8.2, 6.8 Hz, 1H), 7.58 (s, 1H), 7.48 (d, J=10.2 Hz, 1H), 7.20 (dd, J=8.2, 1.6 Hz, 1H), 7.08-6.97 (m, 2H), 6.82 (dd, J=8.4, 2.1 Hz, 1H), 4.68-4.59 (m, 1H), 4.34 (dt, J=13.8, 4.2 Hz, 1H), 3.81 (s, 3H), 3.35 (dd, J=12.8, 9.2 Hz, 1H), 3.29-3.17 (m, 2H), 2.07 (dd, J=12.6, 5.2 Hz, 1H), 1.88-1.79 (m, 1H), 1.67 (q, J=9.8, 6.8 Hz, 1H), 1.54 (td, J=10.8, 5.8 Hz, 1H).

ESI-MS m/z=465.2[M+H]⁺.

### Example 111

### Preparation of 2-fluoro-4-(5-(3-fluoro-4-methoxyphenyl)-2-((3-hydroxy-adamantane-1-yl)amino)-6-methoxypy rimidin-4-yl)benzonitrile trifluoroacetate

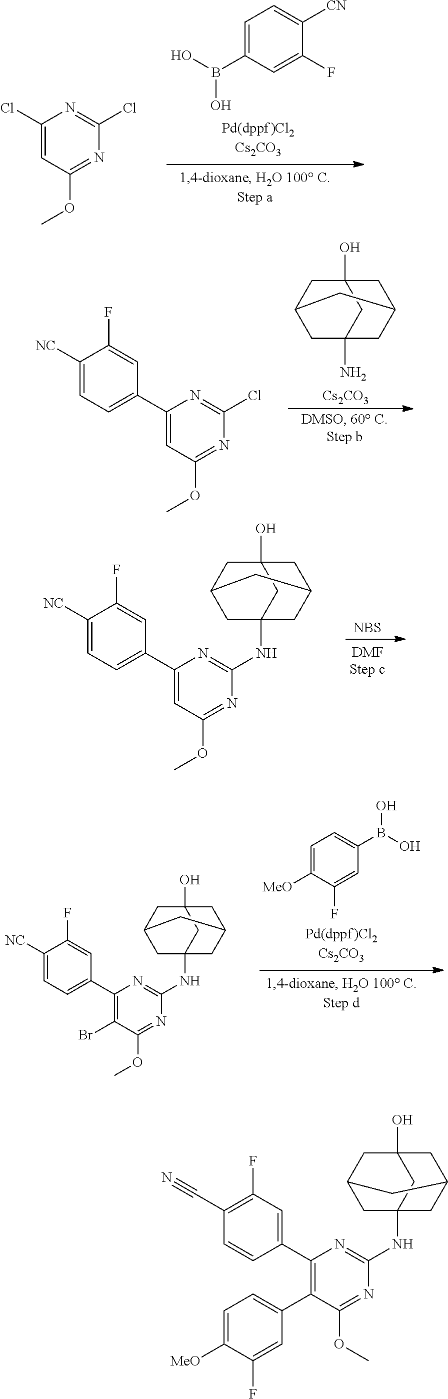

Step a): Preparation of 4-(2-chloro-6-methoxypyrimidin-4-yl)-2-fluorobenzonitrile 2,4-dichloro-6-methoxypyrimidine (750 mg, 4.2 mmol), (4-cyano-3-fluorophenyl)boronic acid (695 mg, 4.2 mmol), Cs₂CO₃ (2.746 g, 8.26 mmol) and Pd(dppf)Cl₂ (155 mg, 0.21 mmol) are added to 15 mL of dioxane and then added with 1.5 ml of water; and the mixed reaction solution is purged by nitrogen blowing and protected with nitrogen. The reaction solution is reacted in microwaves at 100° C. for 60 min, including four parallel reactions; when TLC and LC-MS show that the reaction is completed, the reaction solution is extracted with ethyl acetate (20 mL×3), washed with 20 mL of saline, dried with anhydrous sodium sulfate, filtered, and concentrated to obtain a crude product; and the residue is purified by silica gel chromatography (eluent: petroleum ether/ethyl acetate=10/7) to obtain 4-(2-chloro-6-methoxypyrimidin-4-yl)-2-fluorobenzonitrile with a yield of 20.3%.

¹H NMR (400 MHz, DMSO-d₆) δ ppm 8.32 (t, J=10.6 Hz, 2H), 8.13 (t, J=7.4 Hz, 1H), 7.27 (s, 1H), 4.10 (s, 3H).

ESI-MS m/z=264.0 [M+H]⁺.

### Step b): 2-fluoro-4-(2-(3-hydroxyadamantane-1-yl)amino)-6-methoxypyrimidin-4-yl)benzonitrile

4-(2-chloro-6-methoxypyrimidin-4-yl)-2-fluorobenzonitrile (450 mg, 1.71 mmol), 1-amino-3-hydroxyadamantane (572 mg.3.42 mmol), and Cs₂CO₃ (1.112 g, 3.42 mmol) are dissolved in a dimethyl sulfoxide (20 mL) solution. The solution is reacted at 60° C. for 36 h, and LS-MS shows that the raw materials have been consumed. The reaction solution is quenched with water (20 mL) and extracted with ethyl acetate (20 mL×2); organic layers are combined and then washed with saturated saline (10 mL×2), dried with anhydrous sodium sulfate, filtered, and concentrated under a reduced pressure to obtain a residue; and the residue is purified by silica gel chromatography (eluent: petroleum ether/ethyl acetate=10/7) to obtain 2-fluoro-4-(2-(3-hydroxyadamantane-1-yl)amino)-6-methoxypyrimidin-4-yl)benzonitrile with a yield of 24.5%.

ESI-MS m/z=395.2 [M+H]⁺.

### Step c): 4-(5-bromo-2-((3-hydroxyadamantane-1-yl)amino)-6-methoxypyrimidin-4-yl)-2-fluorobenzonitr ile

2-fluoro-4-(2-(3-hydroxyadamantane-1-yl)amino)-6-methoxypyrimidin-4-yl)benzonitrile (100 mg.0.253 mmol) is dissolved in N,N-dimethylformamide (4 mL), added with NBS (68 mg, 0.381 mmol) at 0° C. and stirred; when LS-MS shows that 2-fluoro-4-(2-(3-hydroxyadamantane-1-yl)amino)-6-methoxypyrimidin-4-yl)benzonitrile has been consumed, the reaction solution is quenched with water (20 mL) and extracted with ethyl acetate (20 mL×2); organic layers are combined and then washed with saturated saline (15 mL×2), dried with anhydrous sodium sulfate, filtered, and concentrated under a reduced pressure to obtain a residue; and the residue is purified by silica gel chromatography (eluent: petroleum ether/ethyl acetate=10/9) to obtain 4-(5-bromo-2-((3-hydroxyadamantane-1-yl)amino)-6-methoxypyrimidin-4-yl)-2-fluorobenzonitr ile with a yield of 98.5%.

ESI-MS m/z=473.1 [M+H]⁺.

### Step d): Preparation of 2-fluoro-4-(5-(3-fluoro-4-methoxyphenyl)-2-((3-hydroxyadamantane-1-yl)amino)-6-methoxypyr imidin-4-yl)benzonitrile trifluoroacetate

4-(5-bromo-2-((3-hydroxyadamantane-1-yl)amino)-6-methoxypyrimidin-4-yl)-2-fluorobenzonitr ile (110 mg, 0.233 mmol), (3-fluoro-4-methylphenyl)boronic acid (60 mg, 0.349 mmol), Cs₂CO₃ (152 mg, 0.466 mmol), and Pd(dppf)Cl₂ (17 mg, 0.0233 mmol) are dissolved in 6 mL of dioxane and added with 0.6 mL of water; and the mixed reaction solution is purged by nitrogen blowing and protected with nitrogen. The reaction solution is reacted in microwaves at 100° C. for 60 min; when TLC and LC-MS show that the reaction is completed, the reaction solution is extracted with ethyl acetate (20 mL×3), washed with 20 mL of saline, dried with anhydrous sodium sulfate, filtered and concentrated to obtain a crude product; and the crude product is delivered to Prep-HPLC preparation (separation method 2) to obtain 2-fluoro-4-(5-(3-fluoro-4-methoxyphenyl)-2-((3-hydroxyadamantane-1-yl)amino)-6-methoxypyr imidin-4-yl)benzonitrile trifluoroacetate with a yield of 50.2%.

¹H NMR (400 MHz, Methanol-d₄) δ ppm 7.67 (t, J=7.4 Hz, 1H), 7.40-7.10 (m, 2H), 7.04-6.82 (m, 2H), 6.76 (d, J=8.4 Hz, 1H), 4.02 (s, 3H), 3.90 (d, J=1.8 Hz, 1H), 3.84 (d, J=1.8 Hz, 3H), 2.32 (s, 2H), 2.20 (d, J=11.8 Hz, 2H), 2.07 (d, J=11.Hz, 2H), 1.70 (d, J=37.6 Hz, 4H).

ESI-MS m/z=519.2[M+H]⁺.

### Example 112

### Preparation of 2-fluoro-4-(5-(3-fluoro-4-methoxyphenyl)-2-((3-hydroxyadamantane-1-yl)amino)-6-carbonyl-1,6-dihydropyrimidin-4-yl)benzonitrile

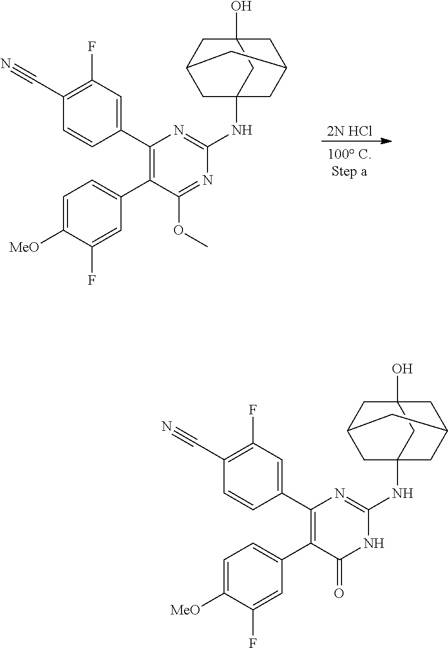

### Step a): Preparation of 2-fluoro-4-(5-(3-fluoro-4-methoxyphenyl)-2-((3-hydroxyadamantane-1-yl)amino)-6-carbonyl-1,6-dihydropyrimidin-4-yl)benzonitrile

2-fluoro-4-(5-(3-fluoro-4-methoxyphenyl)-2-((3-hydroxyadamantane-1-yl)amino)-6-methoxypyr imidin-4-yl)benzonitrile (50 mg, 0.0965 mmol) is added to 4 mL of 2M hydrochloric acid (2 mL of 4M dioxane hydrochloride and 2 mL of water) solution and reacted at 100° C. for 12 h; when LC-MS shows that half of raw materials are reacted, the solvent is dried by spinning, the crude product is delivered to Prep-HPLC (separation method 4) to obtain 2-fluoro-4-(5-(3-fluoro-4-methoxyphenyl)-2-((3-hydroxyadamantane-1-yl)amino)-6-carbonyl-1,6-dihydropyrimidin-4-yl)benzonitrile with a yield of 22.2%.

¹H NMR (400 MHz, Methanol-d₄) δ ppm 7.58 (t, J=7.6 Hz, 1H), 7.28 (dd, J=23.8, 9.4 Hz, 2H), 6.99-6.86 (m, 2H), 6.74 (d, J=8.6 Hz, 1H), 3.83 (d, J=2.4 Hz, 3H), 2.27 (s, 2H), 2.13 (d, J=11.6 Hz, 4H), 2.02 (d, J=11.8 Hz, 2H), 1.65 (d, J=38.2 Hz, 6H).

ESI-MS m/z=505.2 [M+H]⁺.

### Example 113

### 2-fluoro-4-(5-(3-fluoro-4-methoxyphenyl)-6-methoxy-2-(4-methylpiperazin-1-yl)pyrimidin-4-yl) benzonitrile is prepared according to the synthesis method (separation method 4) of Example 111, and the structure and representation data are as follows

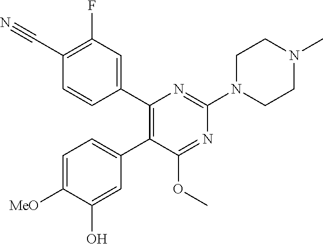 [SMILES: COc1ccc(-c2c(OC)nc(N3CCN(C)CC3)nc2-c2ccc(C#N)c(F)c2)cc1O]

¹H NMR (400 MHz, Methanol-d₄) δ ppm 8.36 (dd, J=39.6, 9.4 Hz, 2H), 7.85 (t, J=7.4 Hz, 1H), 7.24-7.08 (m, 3H), 3.99 (s, 3H), 3.91 (s, 3H), 3.36 (t, J=5.0 Hz, 4H), 2.38 (t, J=5.0 Hz, 4H), 2.26 (s, 3H).

ESI-MS m/z=452.2 [M+H]⁺.

### Example 114

### 2-fluoro-4-(5-(3-fluoro-4-methoxyphenyl)-6-methoxy-2-(3-(methylamino)piperidin-1-yl)pyrimid in-4-yl)benzonitrile hydrochloride is prepared according to the synthesis method (separation method 4) of Example 111, and the structure and representation data are as follows

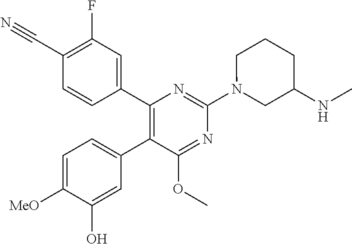 [SMILES: CNC1CCCN(c2nc(OC)c(-c3ccc(OC)c(O)c3)c(-c3ccc(C#N)c(F)c3)n2)C1]

¹H NMR (400 MHz, Methanol-d₄) δ ppm 8.43 (dd, J=8.0, 1.6 Hz, 1H), 8.34 (d, J=10.6 Hz, 1H), 7.87 (t, J=7.4 Hz, 1H), 7.19 (d, J=13.0 Hz, 3H), 4.29 (dd, J=12.4, 3.4 Hz, 1H), 4.00 (s, 3H), 3.92 (s, 3H), 3.51 (dd, J=13.6, 4.2 Hz, 1H), 3.22 (dt, J=8.8, 3.8 Hz, 2H), 3.13 (dd, J=12.6, 9.6 Hz, 1H), 2.85-2.73 (m, 3H), 2.69 (s, 1H), 2.16 (dd, J=11.8, 5.2 Hz, 1H), 1.70-1.38 (m, 3H).

ESI-MS m/z=466.2 [M+H]⁺.

### Example 115

### Preparation of 4-(6-(3-amino-8-azabicyclo[3.2.1]octane-8-carbonyl)-3-(3-hydroxy-4-methoxyphenyl)-4-methox ypyridin-2-yl)-2-fluorobenzonitrile

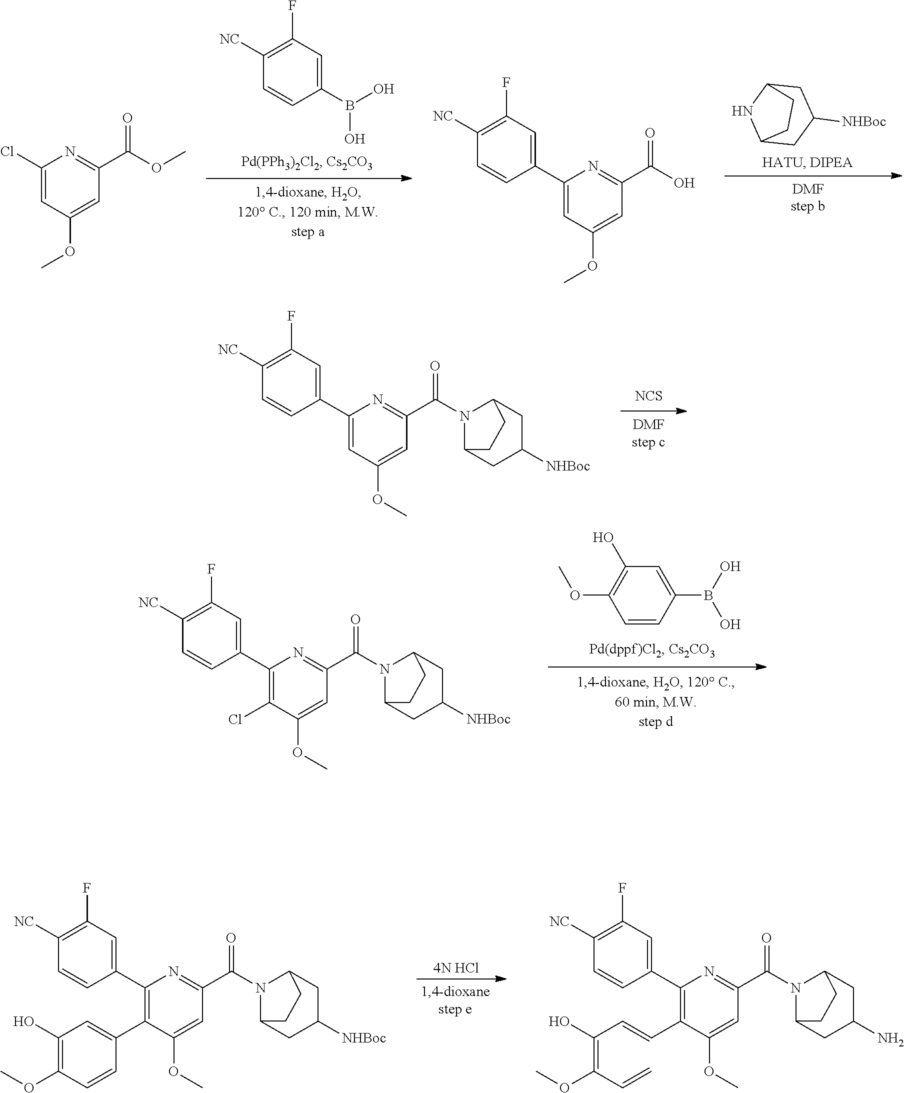

### Step a): Preparation of 6-(4-cyano-3-fluorophenyl)-4-methoxypicolinic acid

Methyl 6-chloro-4-methoxypicolinate (500 mg, 2.488 mmol), (4-cyano-3-fluorophenyl)boronic acid (616 mg, 3.732 mmol), Cs₂CO₃ (1.6 g, 4.976 mmol), Pd(PP₃)Cl₂ (174 mg, 0.249 mmol), 1.4-Dioxane (8 mL) and H₂O (2 mL) are added to a reaction flask, and reacted at 120° C. for 2 h while stirring. The reaction solution is concentrated under a reduced pressure to dryness; and the residue is purified by silica gel chromatography (eluent: dichloromethane/methanol=8/1) to obtain 6-(4-cyano-3-fluorophenyl)-4-methoxypicolinic acid with a yield of 73.0%

ESI-MS(m/z)=273.3[M+H]⁺.

### Step b): Preparation of tert-butyl (8-(6-(4-cyano-3-fluorophenyl)-4-methoxypicolinamido)-8-azabicyclo[3.2.1]octane-3-yl)carbam ate

4-(6-(4-aminopiperidin-1-yl)-4-(3-fluoro-4-methoxyphenyl)-3-methoxypyridin-2-yl)-2-fluoroben zonitrile (496 mg, 1.816 mmol), tert-butyl(8-azabicyclo[3.2.1]octane-3-yl)carbamate (410 mg, 1.816 mmol) and DMF (10 mL) are added to a reaction flask, added with HATU (962 mg, 2.178 mmol) and DIPEA (703 mg, 5.448 mmol) in an ice bath while stirring, and reacted at room temperature for 1 h while stirring. When the reaction is completed, the reaction solution is quenched with water (40 mL), and extracted with ethyl acetate (40 mL×2); organic phase is combined, washed sequentially with saturated sodium bicarbonate aqueous solution (40 mL) and saturated saline (40 mL×2), dried with anhydrous sodium sulfate, and filtered to obtain filtrate; the filtrate is concentrated under a reduced pressure; and the residue is purified by silica gel chromatography (eluent: dichloromethane/methanol=20/1) to obtain tert-butyl(8-(6-(4-cyano-3-fluorophenyl)-4-methoxypicolinamido)-8-azabicyclo[3.2.1]octane-3-y 1)carbamate with a yield of 63.1%.

ESI-MS(m/z)=481.2[M+H]⁺.

### Step c): Preparation of tert-butyl(8-(5-chloro-6-(4-cyano-3-fluorophenyl)-4-methoxypicolinamido)-8-azabicyclo[3.2.1]octane-3-yl)carbamate

Tert-butyl(8-(6-(4-cyano-3-fluorophenyl)-4-methoxypicolinamido)-8-azabicyclo[3.2.1]octane-3-yl)carbamate (550 mg, 1.144 mmol) and DMF (10 mL) are added to a reaction flask, added with NCS in batches (152 mg, 1.144 mmol) in an ice bath, and reacted while stirring at room temperature for 2 h. When the reaction is completed, the reaction solution is added with water (40 mL), and extracted with ethyl acetate (40 mL×3); organic phase is combined, and washed with saturated saline (40 mL×2), and concentrated under a reduced pressure to dryness; and the residue is purified by silica gel chromatography (eluent: dichloromethane/methanol=20/1) to obtain tert-butyl(8-(5-chloro-6-(4-cyano-3-fluorophenyl)-4-methoxypicolinamido)-8-azabicyclo[3.2.1]o ctane-3-yl)carbamate with a yield of 45.0%.

¹H NMR (400 MHz, DMSO-d₆) δppm 8.35-8.17 (m, 2H), 8.09 (dd, J=8.2, 6.8 Hz, 1H), 7.92 (s, 1H), 4.74-4.60 (m, 1H), 4.13 (s, 3H), 3.89 (d, J=38.6 Hz, 1H), 3.65 (s, 1H), 1.97-1.56 (m, 8H), 1.38 (s, 9H).

ESI-MS(m/z)=515.2[M+H]⁺.

### Step d): Preparation of tert-butyl(8-(6-(4-cyano-3-fluorophenyl)-5-(3-hydroxy-4-methoxyphenyl)-4-methoxypicolinami do)-8-azabicyclo[3.2.1]octane-3-yl)carbamate

### (Tert-butyl

8-(5-chloro-6-(4-cyano-3-fluorophenyl)-4-methoxypicolinamido)-8-azabicyclo[3.2.1]octane-3-yl) carbamate (80 mg, 0.155 mmol), (3-hydroxy-4-methoxyphenyl)boronic acid (58 mg, 0.233 mmol), Cs₂CO₃ (101 mg, 0.310 mmol), Pd(dppf)Cl₂ (11 mg, 0.015 mmol), 1.4-Dioxane (4 mL) and H₂O (1 mL) are added to a reaction flask, and reacted at 120° C. for 1 h while stirring. The reaction solution is concentrated under a reduced pressure to dryness; and the residue is purified by silica gel chromatography (eluent: dichloromethane/methanol=15/1) to obtain tert-butyl(8-(6-(4-cyano-3-fluorophenyl)-5-(3-hydroxy-4-methoxyphenyl)-4-methoxypicolinami do)-8-azabicyclo[3.2.1]octane-3-yl)carbamate with a yield of 40.0%.

ESI-MS(m/z)=603.1[M+H]⁺.

### Step e): Preparation of 4-(6-(3-amino-8-azabicyclo[3.2.1]octane-8-carbonyl)-3-(3-hydroxy-4-methoxyphenyl)-4-methox ypyridin-2-yl)-2-fluorobenzonitrile

Tert-butyl(8-(6-(4-cyano-3-fluorophenyl)-5-(3-hydroxy-4-methoxyphenyl)-4-methoxypicolinami do)-8-azabicyclo[3.2.1]octane-3-yl)carbamate (37 mg, 0.062 mmol) is added to a reaction flask, then added with a hydrogen chloride ethyl acetate solution (4M, 2.5 mL) and stirred at room temperature for 1 h; a large amount of solids precipitate, The reaction solution is concentrated under a reduced pressure to obtain a crude product; and the crude product is purified by Prep-HPLC (separation method 4) to obtain 4-(6-(3-amino-8-azabicyclo[3.2.1]octane-8-carbonyl)-3-(3-hydroxy-4-methoxyphenyl)-4-methox ypyridin-2-yl)-2-fluorobenzonitrile with a yield of 54.0%.

¹H NMR (400 MHz, DMSO-d₆) δ ppm 8.46-8.20 (m, 2H), 8.10 (dd, J=8.2, 6.8 Hz, 1H), 7.84 (s, 1H), 6.93 (d, J=8.4 Hz, 1H), 6.87-6.54 (m, 2H), 4.50-4.33 (m, 1H), 3.97 (s, 3H), 3.79 (s, 3H), 3.56 (d, J=6.6 Hz, 1H), 2.95 (tt, J=11.2, 5.6 Hz, 1H), 1.77-1.11 (m, 8H).

ESI-MS(m/z)=503.2[M+H]⁺.

### Example 116

### (3-Amino-8-azabicyclo[3.2.1]octane-8-yl)(6-(3-fluorophenyl)-5-(3-hydroxy-4-methoxyphenyl)-4-methoxypyridin-2-yl)methanone is prepared according to the synthesis method (separation method 4) of Example 115, and the structure and representation data are as follows

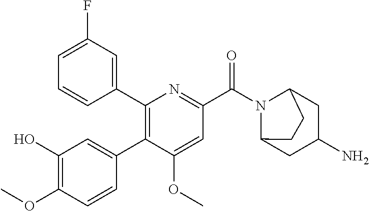 [SMILES: COc1ccc(-c2c(OC)cc(C(=O)N3C4CCC3CC(N)C4)nc2-c2cccc(F)c2)cc1O]

¹H NMR (400 MHz, DMSO-d₆) δ ppm 8.13-7.90 (m, 2H), 7.69 (s, 1H), 7.57 (td, J 8.0, 6.0 Hz, 1H), 7.31 (td, J 8.6, 2.8 Hz, 1H), 6.93 (d, J 8.4 Hz, 1H), 6.79 (d, J 2.0 Hz, 1H), 6.73 (dd, J 8.4, 2.2 Hz, 1H), 4.48-4.25 (m, 1H), 3.95 (s, 3H), 3.79 (s, 3H), 3.58 (dd, J 6.6, 3.2 Hz, 1H), 2.95 (tt, J 11.0, 5.4 Hz, 1H), 1.77-1.13 (m, 8H).

ESI-MS(m/z)=478.2[M+H]⁺.

### Example 117

### Preparation of 4-(5-(4-aminopiperidin-1-yl)-8-(3-fluoro-4-methoxyphenyl)imidazolo[1,2-c]pyrimidin-7-yl]-2-fl uorobenzonitrile hydrochloride

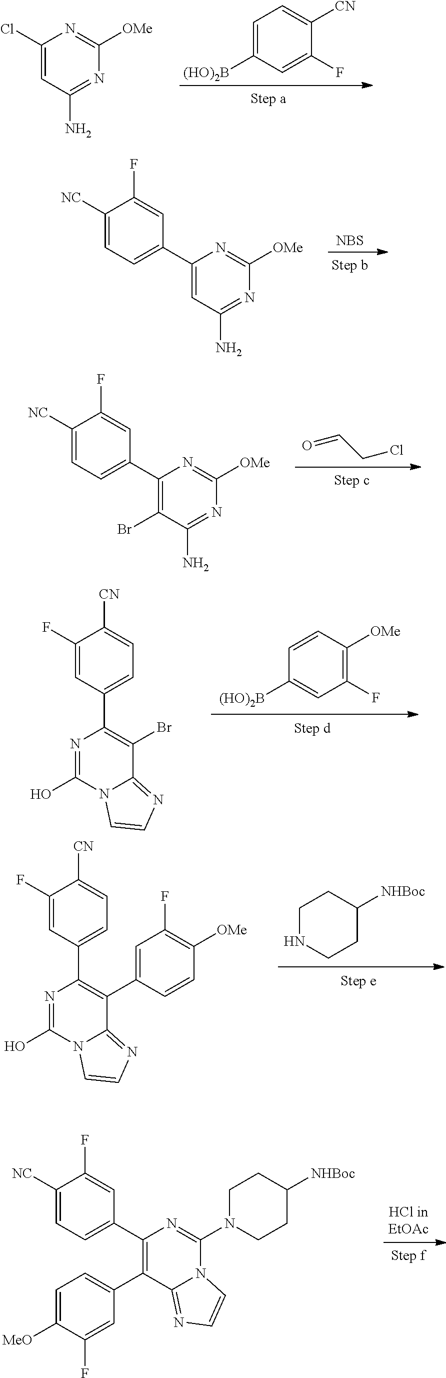

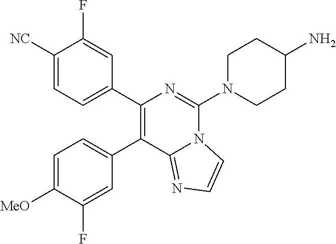 [SMILES: COc1ccc(-c2c(-c3ccc(C#N)c(F)c3)nc(N3CCC(N)CC3)n3ccnc23)cc1F]

Step a): Preparation of 4-(6-amino-2-methoxypyrimidin-4-yl)-2-fluorobenzonitrile 6-chloro-2-methoxypyrimidin-4-amine (3.0 g, 0.03 mol), Na₂CO₃ (9.9 g, 0.09 mol), (4-cyano-3-fluorophenyl)boronic acid (7.8 g, 0.05 mol), and Pd(aphos)₂Cl₂ (4.3 g, 0.006 mol) are dissolved in a 1,4-dioxane aqueous solution (150 ml, 5:1), and the mixed reaction solution is purged by nitrogen blowing and protected with nitrogen. The oil bath is warmed to 95° C. for reaction. When LC-MS shows that the reaction is completed, the reaction solution is concentrated under vacuum, dissolved in ethyl acetate (20 mL), and washed with water (20 mL); aqueous phase is extracted with ethyl acetate (20 mL×3), washed with saturated saline, dried with anhydrous sodium sulfate, filtered, and concentrated; and the residue is purified by silica gel chromatography (eluent: petroleum ether/ethyl acetate=2/1) to obtain 4-(6-amino-2-methoxypyrimidin-4-yl)-2-fluorobenzonitrile with a yield of 88.6%.

ESI-MS m/z: 245.1[M+H]⁺.

### Step b): Preparation of 4-(6-amino-5-bromo-2-methoxypyrimidin-4-yl)-2-fluorobenzonitrile

4-(6-amino-2-methoxypyrimidin-4-yl)-2-fluorobenzonitrile (2.0 g, 0.008 mol) is added to anhydrous DMSO (10 mL) and anhydrous acetonitrile (50 mL) and dissolved, added with NBS (1.45 g, 0.008 mol) under the protection of nitrogen in an ice water bath, and continue to react for 2 h. When LC-MS detection shows that the reaction is completed, the reaction solution is quenched with water (40 mL), then extracted with ethyl acetate (50 mL×3), washed with saturated saline, dried with anhydrous sodium sulfate, filtered, and concentrated; and the residue is purified by silica gel chromatography (eluent: petroleum ether/ethyl acetate=3/1) to obtain 4-(6-amino-5-bromo-2-methoxypyrimidin-4-yl)-2-fluorobenzonitrile with a yield of 78.5%.

ESI-MS m/z: 323.0[M+H]⁺.

### Step c): Preparation of 4-(8-bromo-5-hydroxyimidazopyrazine-7-yl)-2-fluorobenzonitrile

4-(6-amino-5-bromo-2-methoxypyrimidin-4-yl)-2-fluorobenzonitrile (500 mg, 1.553 mmol) is dissolved in isopropanol (13 mL), then added with a raw material chloroacetaldehyde (6.09 g, 31.055 mmol), subjected to nitrogen displacement, and reacted at 100° C. overnight; and the reaction progress is monitored by TLC and LC-MS. When LC-MS shows that the reaction is completed, the reaction solution is concentrated under vacuum, and dissolved in dichloromethane (5 mL); and the residue is purified by silica gel chromatography (eluent: methanol/dichloromethane=1/20) to obtain 4-(8-bromo-5-hydroxyimidazopyrazine-7-yl)-2-fluorobenzonitrile with a yield of 85.7%.

¹H NMR (400 MHz, DMSO-d₆) δ ppm 12.28 (s, 1H), 8.14 (dt, J=9.0, 4.6 Hz, 1H), 7.97 (d, J=2.6 Hz, 1H), 7.84 (dd, J=10.2, 2.6 Hz, 1H), 7.65 (dd, J=7.8, 2.4 Hz, 1H), 7.50 (d, J=2.4 Hz, 1H).

ESI-MS m/z: 333.0[M+H]⁺.

### Step d: Preparation of 2-fluoro-4-(8-(3-fluoro-4-methoxyphenyl)-5-hydroxyimido[1,2-c]pyrimidin-7-yl)benzonitrile

4-(8-bromo-5-hydroxyimidazopyrazine-7-yl)-2-fluorobenzonitrile (380 mg, 1.144 mmol), 3-fluoro-4-methoxyphenylboronic acid (292 mg, 1.717 mmol), Pd(dppf)Cl₂ (167 mg, 0.208 mmol), and Na₂CO₃ (243 mg, 2.289 mmol) are weighed and dissolved in 1,4-dioxane (15 mL) and added with water (3 mL). and the mixed reaction solution is purged by N₂ blowing and protected with N₂, and reacted in a microwave reactor at 105° C. for 30 min; and when LC-MS shows that the reaction is completed, the reaction solution is extracted with ethyl acetate (10 mL×3), washed with 10 ml of saline, dried with anhydrous sodium sulfate, filtered, and concentrated. The residue is purified by silica gel chromatography (eluent: methanol/dichloromethane=1/20) to obtain 2-fluoro-4-(8-(3-fluoro-4-methoxyphenyl)-5-hydroxyimido[1,2-c]pyrimidin-7-yl)benzonitrile with a yield of 89.7%.

¹H NMR (400 MHz, DMSO-d₆) δ ppm 12.02 (s, 1H), 7.94-7.87 (m, 2H), 7.65-7.58 (m, 2H), 7.54-7.50 (m, 1H), 7.28 (dd, J=8.2, 1.6 Hz, 1H), 7.16-7.13 (m, 1H), 7.05 (t, J=8.8 Hz, 1H), 3.81 (s, 3H).

ESI-MS m/z: 379.1[M+H]⁺.

### Step e): Preparation of tert-butyl(1-(7-(4-cyano-3-fluorophenyl)-8-(3-fluoro-4-methoxyphenyl)imidazo[1,2-c]pyrimidin-5-yl)piperidin-4-yl)carbamate

2-fluoro-4-(8-(3-fluoro-4-methoxyphenyl)-5-hydroxyimido[1,2-c]pyrimidin-7-yl)benzonitrile (200 mg, 0.530 mmol), tert-butyl piperidine-4-carbamate (318 mg, 1.57 mmol), and Carter condensation agent (352 mg, 0.795 mmol) are dissolved in 16 mL of anhydrous acetonitrile, and then added with DIPEA (206 mg, 1.590 mmol), subjected to nitrogen displacement, warmed to 60° C. under nitrogen protection and reacted overnight. The reaction progress is monitored by TLC and LC-MS; when the reaction is completed, the reaction solution is cooled and concentrated under vacuum; and the concentrate is directly purified by a thin layer chromatography preparation plate (methanol/dichloromethane=1/10) to obtain tert-butyl(1-(7-(4-cyano-3-fluorophenyl)-8-(3-fluoro-4-methoxyphenyl)imidazo[1,2-c]pyrimidin-5-yl)piperidin-4-yl)carbamate with a yield of 58.4%.

¹H NMR (400 MHz, Methanol-d₄) δ ppm 7.80 (d, J=1.6 Hz, 1H), 7.63-7.55 (m, 2H), 7.45 (dd, J=10.8, 1.6 Hz, 1H), 7.33 (dd, J=8.2, 1.6 Hz, 1H), 7.19-7.06 (m, 2H), 7.00 (dt, J=8.4, 1.6 Hz, 1H), 4.05-3.96 (m, 2H), 3.91 (s, 3H), 3.68 (dq, J=10.6, 6.4, 5.2 Hz, 1H), 3.25-3.15 (m, 2H), 2.07 (dd, J=13.2, 3.8 Hz, 2H), 1.76 (qd, J=11.6, 3.8 Hz, 2H), 1.46 (s, 9H).

ESI-MS m/z: 561.2[M+H]⁺.

### Step f): Preparation of 4-(5-(4-aminopiperidin-1-yl)-8-(3-fluoro-4-methoxyphenyl)imidazo[1,2-c]pyrimidin-7-yl]-2-fluo robenzonitrile hydrochloride

Tert-butyl(1-(7-(4-cyano-3-fluorophenyl)-8-(3-fluoro-4-methoxyphenyl)imidazo[1,2-c]pyrimidin-5-yl)piperidin-4-yl)carbamate (173 mg, 0.309 mmol) is dissolved in a 4M HCl (ethyl acetate solution, 5 mL), and reacted for 40 min at room temperature under nitrogen protection. When LC-MS detection shows that the reaction is completed, the reaction solution is concentrated at room temperature to precipitate solid; the solid is filtered, washed with ethyl acetate, concentrated under vacuum, and freeze-dried to obtain 4-(5-(4-aminopiperidin-1-yl)-8-(3-fluoro-4-methoxyphenyl)imidazo[1,2-c]pyrimidin-7-yl]-2-fluo robenzonitrile hydrochloride with a yield of 23.6%.

¹H NMR (400 MHz, DMSO-d₆) δ ppm 8.48 (d, J=5.2 Hz, 3H), 8.20 (d, J=2.0 Hz, 1H), 8.07 (s, 1H), 7.88 (t, J=7.6 Hz, 1H), 7.58 (dd, J=10.6, 1.6 Hz, 1H), 7.34-7.27 (m, 2H), 7.24 (t, J=8.6 Hz, 1H), 7.06-6.97 (m, 1H), 4.09 (d, J=13.2 Hz, 2H), 3.89 (s, 3H), 3.38 (d, J=12.2 Hz, 1H), 3.27 (t, J=12.6 Hz, 2H), 2.19-2.05 (m, 2H), 1.90 (qd, J=12.2, 3.8 Hz, 2H).

ESI-MS m/z: 461.2[M+H]⁺.

Compounds in Examples 118-186 are prepared according to the synthesis method of Example 117 (separation method for the compounds: free alkali, hydrochloride and formate are separately prepared according to separation methods 4, 1 and 3, respectively), and their structures and characterization data are as follows:

| | | | | MS |
|---|---|---|---|---|
| | | | | (M + |
| Examples | Chemical name | Structure | ¹H NMR | H)⁺ |
| 118 | 4-(5-(3-amino- 8-azabicyclo [3.2.1]octane-8- yl)-8-(5-fluoro-3- methylbenzo[d] isoxazol-6-yl) imidazolo[1,2 c]pyrimidin-7- yl)-2- fluorobenzonitrile | 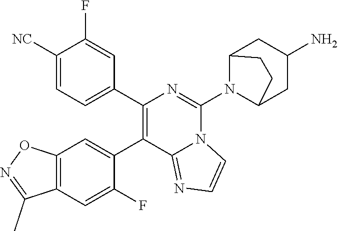 [SMILES: Cc1noc2cc(-c3c(-c4ccc(C#N)c(F)c4)nc(N4C5CCC4CC(N)C5)n4ccnc34)c(F)cc12] | ¹H NMR (400 MHz, Methanol-d₄) δ ppm 7.93 (s, 1H), 7.71 (d, J = 5.2 Hz, 1H), 7.68 − 7.54 (m, 2H), 7.54 − 7.43 (m, 2H), 7.31 (d, J = 8.2 Hz, 1H), 4.84 (s, 2H), 3.31 (q, J = 1.8 Hz, 1H), 2.58 (s, 3H), 2.20 (t, J = 7.2 Hz, 2H), 2.05 (d, J = 12.8 Hz, 2H), 1.98 − 1.77 (m, 4H). | |
| 119 | 2-fluoro-4-(8- (3-fluoro-4- methoxyphenyl)- 5-(3- (methylamino) piperidin-1-yl) imidazolo[1,2-c] pyrimidin-7-yl) benzonitrile | 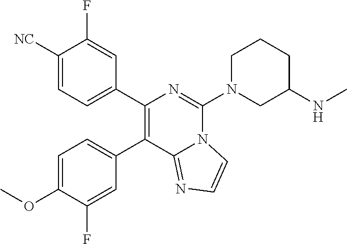 | ¹H NMR (400 MHz, DMSO-d₆) δ ppm 7.91 (s, 1H), 7.82 (t, J = 7.4 Hz, 1H), 7.67 (s, 1H), 7.53 (d, J = 10.8 Hz, 1H), 7.36 − 7.22 (m, 2H), 7.14 (t, J = 8.6 Hz, 1H), 7.02 (d, J = 8.4 Hz, 1H), 3.86 (s, 3H), 3.74 (d, J = 12.8 Hz, 1H), 3.29 (s, 1H), 3.14 (t, J = 11.0 Hz, 1H), 2.97 − 2.87 (m, 1H), 2.78 (s, 1H), 2.36 (s, 3H), 1.93 (dd, J = 45.4, 11.6 Hz, 2H), 1.72 (d, J = 13.0 Hz, 1H), 1.40 (d, J = 11.2 Hz, 1H). | 475.2 |
| 120 | 4-(5-(3-amino-8- azabicyclo[3.2.1] octane-8-yl)-8- (3-fluoro-4- methoxyphenyl) imidazolo[1,2-c] pyrimidin-7-yl)-2- fluorobenzonitrile | 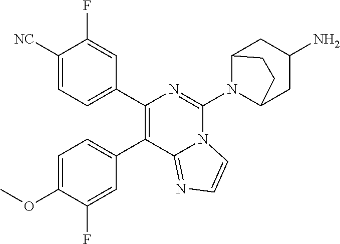 [SMILES: COc1ccc(-c2c(-c3ccc(C#N)c(F)c3)nc(N3C4CCC3CC(N)C4)n3ccnc23)cc1F] | ¹H NMR (400 MHz, DMSO-d₆) δ ppm 7.92 (s, 1H), 7.82 (t, J = 7.6 Hz, 1H), 7.65 (s, 1H), 7.49 (d, J = 10.9 Hz, 1H), 7.36 − 7.19 (m, 2H), 7.13 (t, J = 8.8 Hz, 1H), 7.00 (d, J = 8.4 Hz, 1H), 4.76 − 4.41 (m, 2H), 3.86 (s, 3H), 3.14 (tt, J = 11.8, 5.4 Hz, 1H), 2.27 − 1.72 (m, 8H), 1.65 (t, J = 11.8 Hz, 2H). | 487.2 |
| 121 | 4-(5-(4- aminopiperidin- 1-yl)-8-(6- methoxypyridin- 3-yl)imidazolo [1,2-c]pyrimidin- 7-yl)-2- fluorobenzonitrile | 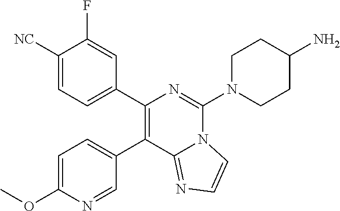 [SMILES: COc1ccc(-c2c(-c3ccc(C#N)c(F)c3)nc(N3CCC(N)CC3)n3ccnc23)cn1] | ¹H NMR (400 MHz, DMSO-d₆) δ ppm 8.08 (d, J = 2.4 Hz, 1H), 7.93 − 7.74 (m, 2H), 7.74 − 7.60 (m, 2H), 7.55 (d, J = 10.8 Hz, 1H), 7.31 (d, J = 8.0 Hz, 1H), 6.86 (d, J = 8.4 Hz, 1H), 3.88 (d, J = 10.2 Hz, 5H), 3.09 (t, J = 11.8 Hz, 2H), 2.89 (tt, J = 9.4, 4.0 Hz, 1H), 2.29 − 1.80 (m, 4H), 1.52 (q, J = 9.8 Hz, 2H). | 444.2 |
| 122 | 4-(5-(4- aminopiperidin-1- yl)-8-(3-fluoro-4- methoxyphenyl) ethynyl)imidazolo [1,2-c]pyrimidin- 7-yl)-2- fluorobenzonitrile hydrochloride | 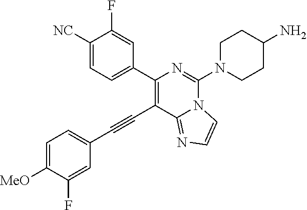 [SMILES: COc1ccc(C#Cc2c(-c3ccc(C#N)c(F)c3)nc(N3CCC(N)CC3)n3ccnc23)cc1F] | ¹H NMR (400 MHz, DMSO-d6 + D20) δ ppm 8.31 − 8.15 (m, 2H), 8.12 (t, J = 7.2 Hz, 1H), 8.01 (s, 1H), 7.59 (dd, J = 21.0, 8.6 Hz, 1H), 7.41 (dd, J = 26.8, 10.2 Hz, 2H), 7.25 (t, J = 8.8 Hz, 1H), 4.13 (d, J = 13.4 Hz, 2H), 3.88 (d, J = 1.6 Hz, 3H), 3.38 (s, 1H), 3.23 (t, J = 12.6 Hz, 2H), 2.05 (d, J = 12.2 Hz, 2H), 1.79 (q, J = 11.2, 9.8 Hz, 2H | 485.2 |
| 123 | 4-(5-(4- aminopiperidin-1- yl)-8-(3-chloro- 1H-indazolo-5-yl) imidazolo[1,2-c] pyrimidin-7-yl)-2- fluorobenzonitrile | 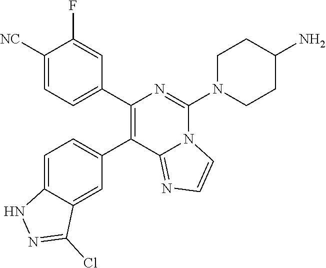 [SMILES: N#Cc1ccc(-c2nc(N3CCC(N)CC3)n3ccnc3c2-c2ccc3[nH]nc(Cl)c3c2)cc1F] | ¹H NMR (400 MHz, DMSO-d₆) δ ppm 7.89 (s, 1H), 7.84 − 7.70 (m, 2H), 7.67 (s, 1H), 7.53 (dd, J = 10.0, 5.4 Hz, 2H), 7.25 (dd, J = 8.4, 6.2 Hz, 2H), 3.92 (d, J = 12.8 Hz, 2H), 3.11 (t, J = 12.2 Hz, 2H), 2.90 (s, 1H), 1.91 (d, J = 12.8 Hz, 2H), 1.54 (q, J = 11.2 Hz, 2H). | 487.2 |
| 124 | 4-(5-(4- aminopiperidin-1- yl)-8-3-hydroxy-4- (methoxyphenyl) imidazolo[1,2-c] pyrimidin-7- yl)-2- fluorobenzonitrile | 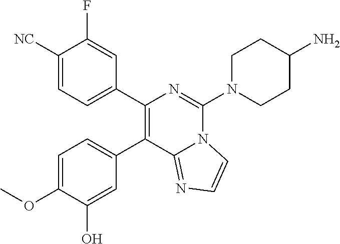 [SMILES: COc1ccc(-c2c(-c3ccc(C#N)c(F)c3)nc(N3CCC(N)CC3)n3ccnc23)cc1O] | ¹H NMR (400 MHz, DMSO-d₆) δ ppm 9.04 (s, 1H), 7.94 − 7.75 (m, 2H), 7.64 (d, J = 1.2 Hz, 1H), 7.49 (dd, J = 11.0, 1.6 Hz, 1H), 7.34 (dd, J = 8.2, 1.6 Hz, 1H), 6.97 − 6.72 (m, 2H), 6.65 (dd, J = 8.4, 2.0 Hz, 1H), 3.88 (d, J = 12.8 Hz, 2H), 3.79 (s, 3H), 3.07 (t, J = 11.8 Hz, 2H), 2.98 − 2.80 (m, 1H), 2.60 (s, 2H), 1.90 (d, J = 11.8Hz, 2H), 1.53 (q, J = 10.8 Hz, 2H). | 459.2 |
| 125 | 4-(5-(4- aminopiperidin- 1-yl)-8-(5-fluoro- 3-methylbenzo[d] isoxazol-6-yl) imidazolo[1,2-c] pyrimidin-7-yl)-2- fluorobenzonitrile | 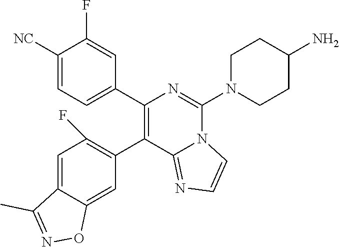 [SMILES: Cc1noc2cc(-c3c(-c4ccc(C#N)c(F)c4)nc(N4CCC(N)CC4)n4ccnc34)c(F)cc12] | ¹H NMR (400 MHz, DMSO-d₆) δ ppm 8.03 − 7.85 (m, 2H), 7.79 (dd, J = 8.4, 6.8 Hz, 2H), 7.71 − 7.51 (m, 2H), 7.29 (dd, J = 8.2, 1.6 Hz, 1H), 4.13 − 3.90 (m, 2H), 3.15 (td, J = 13.0, 12.4, 2.6 Hz, 2H), 2.92 (dt, J = 10.2, 5.8 Hz, 1H), 2.57 (s, 3H), 2.04 − 1.80 (m, 2H), 1.54 (s, 2H). | 486.2 |
| 126 | 4-(5-(4- aminopiperidin- 1-yl)-8- (3-fluoro-4- (3-hydroxy-3- methyl-1-alkynyl- 1-yl)phenyl) imidazolo[1,2-c] pyrimidin-7-yl)-2- fluorobenzonitrile | 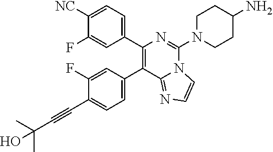 [SMILES: CC(C)(O)C#Cc1ccc(-c2c(-c3ccc(C#N)c(F)c3)nc(N3CCC(N)CC3)n3ccnc23)cc1F] | ¹H NMR (400 MHz, Methanol-d₄) δ ppm 8.21 (d, J = 2.4 Hz, 1H), 8.03 (d, J = 2.2 Hz, 1H), 7.72 (dd, J = 8.2, 6.8 Hz, 1H), 7.65 − 7.52 (m, 2H), 7.41 − 7.26 (m, 2H), 7.17 (dd, J = 7.8, 1.6 Hz, 1H), 4.31 (d, J = 13.4 Hz, 2H), 3.64 − 3.50 (m, 1H), 3.50 − 3.38 (m, 2H), 2.24 (d, J = 12.0 Hz, 2H), 2.02 (qd, J = 12.4, 4.0 Hz, 2H), 1.61 (s, 6H) | 513.2 |
| | hydrochloride | | | |
| 127 | 4-(5-(4- aminopiperidin- 1-yl)-8-(5-fluoro- 1-(2-hydroxy-2- methylpropyl)- 1H-indazo-6-yl) imidazolo[1,2-c] pyrimidin-7-yl)-2- fluorobenzonitrile hydrochloride | 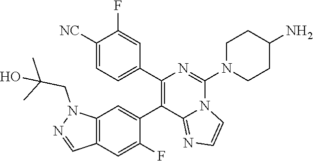 [SMILES: CC(C)(O)Cn1ncc2cc(F)c(-c3c(-c4ccc(C#N)c(F)c4)nc(N4CCC(N)CC4)n4ccnc34)cc21] | ¹H NMR (400 MHz, DMSO-d₆) δ ppm 8.48 − 8.33 (m, 3H), 8.29 − 8.21 (m, 1H), 8.14 (s, 1H), 8.09 (s, 1H), 7.85 − 7.74 (m, 2H), 7.69 (d, J = 9.8 Hz, 1H), 7.55 (dd, J = 10.6, 1.4 Hz, 1H), 7.30 (dd, J = 8.0, 1.4 Hz, 1H), 4.39 − 4.08 (m, 4H), 3.54 − 3.20 (m, 2H), 2.13 (d, J = 12.4 Hz, 2H), 2.03 − 1.81 (m, 2H), 1.24 (s, 1H), 1.05 (s, 3H), 0.95 (s, 3H). | 543.2 |
| 128 | 4-(5-(4- aminopiperidin-1- yl)-8-(4- morpholinophenyl) imidazolo [1,2-c]pyrimidin- 7-yl)-2- fluorobenzonitrile | 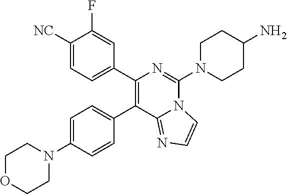 [SMILES: N#Cc1ccc(-c2nc(N3CCC(N)CC3)n3ccnc3c2-c2ccc(N3CCOCC3)cc2)cc1F] | ¹H NMR (400 MHz, DMSO-d₆) δ ppm 7.93 − 7.74 (m, 2H), 7.64 (d, J = 1.4 Hz, 1H), 7.48 (dd, J = 11.0, 1.6 Hz, 1H), 7.33 (dd, J = 8.0, 1.6 Hz, 1H), 7.20 (d, J = 8.8 Hz, 2H), 7.00 − 6.82 (m, 2H), 4.00 − 3.81 (m, 2H), 3.75 (dd, J = 5.8, 3.8 Hz, 4H), 3.22 − 2.99 (m, 6H), 2.88 (dt, J = 9.8, 5.4 Hz, 1H), 1.89 (d, J = 11.4 Hz, 2H), 1.52 (q, J = 9.8 Hz, 2H). | 498.2 |
| 129 | 4-(5-(4- aminopiperidin-1- yl)-8-(2-oxo-2,3- dihydrobenzo[d] oxazol-5-yl) imidazolo[1,2-c] pyrimidin-7-yl)-2- fluorobenzonitrile | 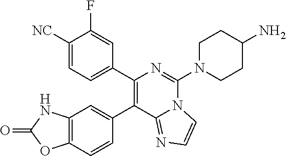 [SMILES: N#Cc1ccc(-c2nc(N3CCC(N)CC3)n3ccnc3c2-c2ccc3oc(=O)[nH]c3c2)cc1F] | ¹H NMR (400 MHz, DMSO-d₆) δ ppm 7.85 (d, J = 1.6 Hz, 1H), 7.77 (dd, J = 8.2, 7.0 Hz, 1H), 7.63 (d, J = 1.4 Hz, 1H), 7.53 (dd, J = 11.0, 1.6 Hz, 1H), 7.28 (dd, J = 8.0, 1.6 Hz, 1H), 7.21 − 7.04 (m, 2H), 6.82 (dd, J = 8.2, 1.8 Hz, 1H), 3.92 (d, J = 13.0 Hz, 2H), 3.11 (d, J = 11.2 Hz, 2H), 2.99 (dt, J = 10.2, 6.2 Hz, 1H), 1.94 (d, J = 11.4 Hz, 2H), 1.60 (q, J = 10.4 Hz, 2H). | 470.2 |
| 130 | 4-(5-(4- aminopiperidin-1- yl)-8-(3-fluoro-4- (trifluoromethoxy) phenyl)imidazolo [1,2-c]pyrimidin- 7-yl)-2- fluorobenzonitrile | 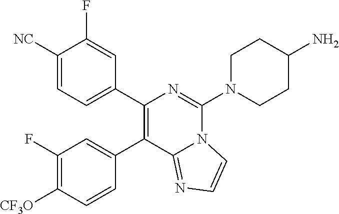 | ¹H NMR (400 MHz, DMSO-d₆) δ ppm 7.91 (d, J = 1.6 Hz, 1H), 7.89 − 7.80 (m, 1H), 7.68 (d, J = 1.4 Hz, 1H), 7.67 − 7.45 (m, 3H), 7.31 (dd, J = 8.2, 1.6 Hz, 1H), 7.19 (dt, J = 8.6, 1.4 Hz, 1H), 3.94 (d, J = 12.8 Hz, 2H), 3.12 (t, J = 11.4 Hz, 2H), 2.91 (dd, J = 10.8, 6.8 Hz, 1H), 1.90 (dd, J = 13.6, 3.8 Hz, 2H), 1.53 (q, J = 10.0 Hz, 2H). | 515.2 |
| 131 | 4-(5-(4- aminopiperidin-1- yl)-8-(imidazolo [1,2-a]pyridin-7-yl) imidazolo[1,2-c] pyrimidin-7-yl)-2- fluorobenzonitrile | 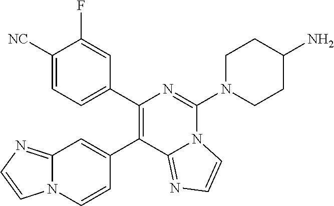 [SMILES: N#Cc1ccc(-c2nc(N3CCC(N)CC3)n3ccnc3c2-c2ccn3ccnc3c2)cc1F] | ¹H NMR (400 MHz, DMSO-d₆) δ ppm 8.51 (dd, J = 7.0, 1.0 Hz, 1H), 7.98 (s, 1H), 7.90 (d, J = 1.6 Hz, 1H), 7.82 (dd, J = 8.0, 7.0 Hz, 1H), 7.75 − 7.51 (m, 4H), 7.40 (dd, J = 8.0, 1.6 Hz, 1H), 6.75 (dd, J = 7.0, 1.6 Hz, 1H), 3.93 (dd, J = 13.0, 3.8 Hz, 2H), 3.12 (ddd, J = 13.6, 11.2, 2.6 Hz, 2H), 2.98 − 2.82 (m, 1H), 1.95 − 1.87 (m, 2H), 1.59 − 1.47 (m, 2H). | 453.2 |
| 132 | 4-(5-(4- aminopiperidin- 1-yl)-8-(2-fluoro- 4-(2-hydroxy-2- methylpropyl) phenyl) imidazolo[1,2-c] pyrimidin-7-yl)-2- fluorobenzonitrile | 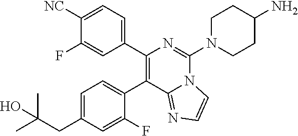 [SMILES: CC(C)(O)Cc1ccc(-c2c(-c3ccc(C#N)c(F)c3)nc(N3CCC(N)CC3)n3ccnc23)c(F)c1] | ¹H NMR (400 MHz, DMSO-d₆) δ ppm 8.32 (s, 3H), 8.15 (s, 1H), 7.92 (q, J = 7.6 Hz, 2H), 7.50 (dd, J = 10.8, 1.6 Hz, 1H), 7.43 (dd, J = 8.0, 1.6 Hz, 1H), 7.36 (t, J = 7.9 Hz, 1H), 7.27 − 7.14 (m, 2H), 4.18 (d, J = 13.4 Hz, 3H), 3.46 (d, J = 4.8 Hz, 1H), 3.37 − 3.21 (m, 2H), 2.79 (s, 2H), 2.25 − 2.10 (m, 2H), 2.00 − 1.82 (m, 2H), 1.16 (s, 6H). | 503.2 |
| | hydrochloride | | | |
| 133 | 4-(5-(4- aminopiperidin-1- yl)-8-(3-hydroxy- 4-(trifluoromethoxy) phenyl)imidazolo [1,2-c]pyrimidin- 7-yl)-2- fluorobenzonitrile hydrochloride | 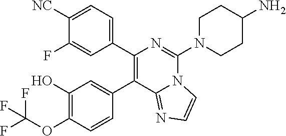 [SMILES: N#Cc1ccc(-c2nc(N3CCC(N)CC3)n3ccnc3c2-c2ccc(OC(F)(F)F)c(O)c2)cc1F] | ¹H NMR (400 MHz, Methanol-d₄) δ ppm 8.09 (d, J = 2.2 Hz, 1H), 7.90 (d, J = 2.2 Hz, 1H), 7.60 (dd, J = 8.2, 6.6 Hz, 1H), 7.42 (dd, J = 10.4, 1.6 Hz, 1H), 7.30 (ddd, J = 8.4, 6.8, 1.4 Hz, 2H), 6.92 (d, J = 2.0 Hz, 1H), 6.88 − 6.71 (m, 1H), 4.18 (d, J = 13.6 Hz, 2H), 3.55 − 3.40 (m, 1H), 3.40 − 3.27 (m, 2H), 2.18 − 2.01 (m, 2H), 1.91 (qd, J = 12.4, 4.0 Hz, 2H). | 513.2 |
| 134 | 4-(5-(4- aminopiperidin-1- yl)-8-(3-fluoro-4- (2-hydroxy-2- methylpropyl) phenyl)imidazolo [1,2-c]pyrimidin- 7-yl)-2- fluorobenzonitrile | 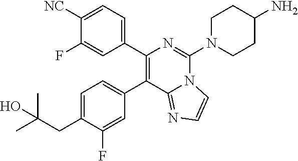 [SMILES: CC(C)(O)Cc1ccc(-c2c(-c3ccc(C#N)c(F)c3)nc(N3CCC(N)CC3)n3ccnc23)cc1F] | ¹H NMR (400 MHz, DMSO-d₆) δ ppm 7.88 (d, J = 1.6 Hz, 1H), 7.81 (dd, J = 8.2, 7.0 Hz, 1H), 7.67 (d, J = 1.4 Hz, 1H), 7.48 − 7.34 (m, 2H), 7.29 (t, J = 8.0 Hz, 1H), 7.17 (dd, J = 10.8, 1.8 Hz, 1H), 7.00 (dd, J = 7.8, 1.8 Hz, 1H), 4.46 (s, 1H), 3.92 (dt, J = 13.4, 4.0 Hz, 2H), 3.16 − 3.04 (m, 2H), 2.90 (tt, J = 9.2, 4.0 Hz, 1H), 2.73 (s, 2H), 1.90 (dd, J = 13.2, 3.8 Hz, 2H), 1.64 − 1.47 (m, 2H), 1.10 (s, 6H). | 503.2 |
| 135 | 4-(5-(4- aminopiperidin- 1-yl)-8-(3-cyano- 4-methoxyphenyl) imidazolo[1,2-c] pyrimidin-7-yl)-2- fluorobenzonitrile | 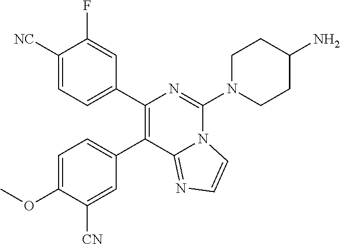 [SMILES: COc1ccc(-c2c(-c3ccc(C#N)c(F)c3)nc(N3CCC(N)CC3)n3ccnc23)cc1C#N] | ¹H NMR (400 MHz, DMSO-d₆) δ ppm 7.92 (m, 1H), 7.88 − 7.76 (m, 2H), 7.69 (d, J = 1.4 Hz, 1H), 7.55 (ddd, J = 16.4, 9.8, 1.8 Hz, 2H), 7.36 − 7.11 (m, 2H), 3.94 (s, 2H), 2.60 (s, 3H), 3.20 − 3.00 (m, 3H), 1.96 (d, J = 11.8 Hz, 2H), 1.64 (q, J = 10.2 Hz, 2H). | 468.2 |
| 136 | 4-(5-(4- aminopiperidin- 1-yl)-8-(3-hydroxy- 4-methylphenyl) imidazolo[1,2-c] pyrimidin-7-yl)-2- fluorobenzonitrile hydrochloride | 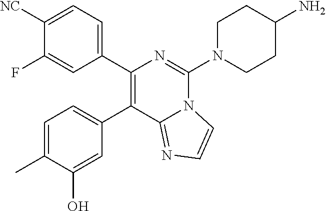 [SMILES: Cc1ccc(-c2c(-c3ccc(C#N)c(F)c3)nc(N3CCC(N)CC3)n3ccnc23)cc1O] | ¹H NMR (400 MHz, Methanol-d₄) δ ppm 8.11 (d, J = 2.2 Hz, 1H), 7.90 (d, J = 1.8 Hz, 1H), 7.58 (dd, J = 8.2, 6.8 Hz, 1H), 7.46 (d, J = 1.4 Hz, 1H), 7.36 (dd, J = 8.2, 1.4 Hz, 1H), 7.16 (d, J = 7.6 Hz, 1H), 6.68 (d, J = 6.4 Hz, 2H), 4.17 (d, J = 13.2 Hz, 2H), 3.46 (tt, J = 11.8, 3.6 Hz, 1H), 3.32 (t, J = 12.6 Hz, 2H), 2.17 (s, 5H), 1.94 (qt, J = 16.8, 8.2 Hz, 2H). | 443.2 |
| 137 | 4-(5-(4- aminopiperidin- 1-yl)-8- (4-fluoro-3- hydroxyphenyl) imidazolo[1,2-c] pyrimidin-7-yl]-2- fluorobenzonitrile | 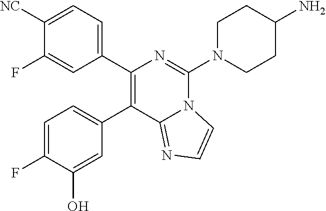 [SMILES: N#Cc1ccc(-c2nc(N3CCC(N)CC3)n3ccnc3c2-c2ccc(F)c(O)c2)cc1F] | ¹H NMR (400 MHz, DMSO-d₆) δ ppm 7.90 − 7.78 (m, 2H), 7.66 (d, J = 1.4 Hz, 1H), 7.51 (dd, J = 10.8, 1.6 Hz, 1H), 7.30 (dd, J = 8.2, 1.6 Hz, 1H), 7.11 (dd, J = 11.2, 8.2 Hz, 1H), 7.00 (dd, J = 8.6, 2.2 Hz, 1H), 6.67 (ddd, J = 8.4, 4.4, 2.2 Hz, 1H), 3.92 (d, J = 13.4 Hz, 2H), 3.10 (t, J = 11.6 Hz, 2H), 2.96 (dt, J = 10.4, 6.2 Hz, 1H), 1.97 − 1.86 (m, 2H), 1.65 − 1.48 (m, 2H). | 447.2 |
| 138 | 2-fluoro-4-(8- (3-hydroxy-4- methoxyphenyl)- 5-(4- (methylamino) piperidin-1-yl) imidazolo[1,2- c]pyrimidin-7- yl)benzonitrile hydrochloride | 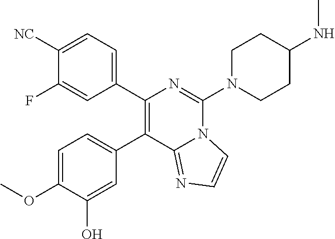 [SMILES: CNC1CCN(c2nc(-c3ccc(C#N)c(F)c3)c(-c3ccc(OC)c(O)c3)c3nccn23)CC1] | ¹H NMR (400 MHz, Methanol-d₄) δ ppm 8.24 (s, 1H), 8.04 (s, 1H), 7.70 (dd, J = 8.2, 6.6 Hz, 1H), 7.57 (s, 1H), 7.55 − 7.44 (m, 1H), 7.11 (d, J = 7.7 Hz, 1H), 6.86 (s, 2H), 4.32 (d, J = 13.2 Hz, 2H), 3.95 (s, 3H), 3.55 − 3.47 (m, 1H), 3.42 (t, J = 12.5 Hz, 2H), 2.80 (s, 3H), 2.33 (d, J = 12.0 Hz, 2H), 2.13 − 1.98 (m, 2H). | 473.2 |
| 139 | (R)-4-(5-(3- aminopiperidin- 1-yl)-8-(3- hydroxy-4- methoxyphenyl) imidazolo[1,2-c] pyrimidin- 7-yl)-2- fluorobenzonitrile hydrochloride | 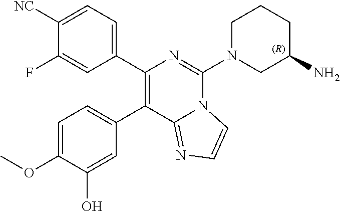 | ¹H NMR (400 MHz, Methanol-d₄) δ ppm 8.26 (d, J = 2.2 Hz, 1H), 8.06 (d, J = 2.2 Hz, 1H), 7.71 (dd, J = 8.2, 6.8 Hz, 1H), 7.57 (dd, J = 10.5, 1.4 Hz, 1H), 7.49 (dd, J = 8.2, 1.2 Hz, 1H), 7.12 (d, J = 8.0 Hz, 1H), 6.86 (d, J = 7.4 Hz, 2H), 4.25 (dd, J = 13.2, 3.4 Hz, 1H), 3.95 (s, 4H), 3.77 (dt, J = 8.7, 4.8 Hz, 1H), 3.56 (ddd, J = 20.2, 13.4, 9.6 Hz, 2H), 2.35 − 2.23 (m, 1H), 2.21 − 2.09 (m, 1H), 2.07 − 1.85 (m, 2H). | 459.2 |
| 140 | 4-(5-(4 (dimethylamino) piperidin-1-yl)-8- (3-hydroxy-4- methoxyphenyl) imidazolo[1,2-c] pyrimidin-7-yl)-2- fluorobenzonitrile | 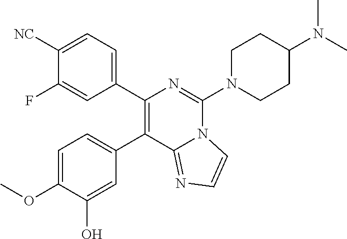 | ¹H NMR (400 MHz, DMSO-d₆) δ ppm 9.04 (s, 1H), 7.88 (d, J = 1.4 Hz, 1H), 7.81 (dd, J = 8.2, 7.0 Hz, 1H), 7.65 (d, J = 1.4 Hz, 1H), 7.48 (dd, J = 11.2, 1.6 Hz, 1H), 7.34 (dd, J = 8.2, 1.6 Hz, 1H), 6.89 (d, J = 8.4 Hz, 1H), 6.83 (d, J = 2.2 Hz, 1H), 6.66 (dd, J = 8.2, 2.2 Hz, 1H), 3.96 (d, J = 12.8 Hz, 2H), 3.79 (s, 3H), 3.00 (t, J = 12.2 Hz, 2H), 2.43 − 2.31 (m, 1H), 2.24 (s, 6H), 2.00 − 1.89 (m, 2H), 1.67 (dd, J = 13.8, 10.2 Hz, 2H). | 487.2 |
| 141 | 4-(5-(4- aminopiperidin-1- yl)-8-(4-hydroxy- 3-methoxyphenyl) imidazolo[1,2-c] pyrimidin-7-yl)- 2- fluorobenzonitrile formate | 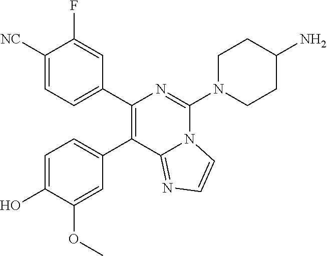 [SMILES: COc1cc(-c2c(-c3ccc(C#N)c(F)c3)nc(N3CCC(N)CC3)n3ccnc23)ccc1O] | ¹H NMR (400 MHz, DMSO-d₆) δ ppm: 9.21 (s, 1H),8.54 (s, 1H), 7.91 (d, J = 1.6 Hz, 1H), 7.81 (dd, J = 8.0, 7.0 Hz, 1H), 7.69 (d, J = 1.4 Hz, 1H), 7.50 (dd, J = 11.0, 1.5 Hz, 1H), 7.32 (dd, J = 8.2, 1.5 Hz, 1H), 6.89 (d, J = 2.0 Hz, 1H), 6.81 − 6.68 (m, 2H), 3.99 (d, J = 13.2 Hz, 2H), 3.61 (s, 3H), 3.31 (d, J = 8.4 Hz 1H), 3.13 (t, J = 12.4 Hz, 2H), 2.03 (d, J = 11.8 Hz, 2H), 1.79 (d, J = 12.0 Hz, 2H). | 459.2 |
| 142 | 4-(5-(4- aminopiperidin-1- yl)-8-(4-chloro- 3-hydroxyphenyl) imidazolo[1,2-c] pyrimidin-7-yl)-2- fluorobenzonitrile formate | 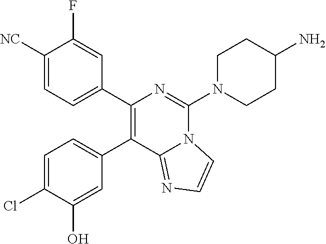 [SMILES: N#Cc1ccc(-c2nc(N3CCC(N)CC3)n3ccnc3c2-c2ccc(Cl)c(O)c2)cc1F] | ¹H NMR (400 MHz, DMSO-d₆) δ ppm 8.33 (s, 1H), 7.91 (d, J = 1.6 Hz, 1H), 7.83 (t, J = 7.6 Hz, 1H), 7.68 (d, J = 1.4 Hz, 1H), 7.57 (dd, J = 10.8, 1.6 Hz, 1H), 7.32 − 7.28 (m, 2H), 7.04 (d, J = 2.0 Hz, 1H), 6.68 (dd, J = 8.2, 2.0 Hz, 1H), 4.02 (d, J = 13.2 Hz, 2H), 3.22 (s, 1H), 3.13 (d, J = 12.2 Hz, 2H), 2.02 (d, J = 12.6 Hz, 2H), 1.70 (q, J = 12.0, 10.4 Hz, 2H). | 463.1 |
| 143 | 4-(5-(4-amino-4- methylpiperidin-1- yl)-8-(3-hydroxy- 4-methoxyphenyl) imidazolo[1,2-c] pyrimidin-7-yl)-2- fluorobenzonitrile | 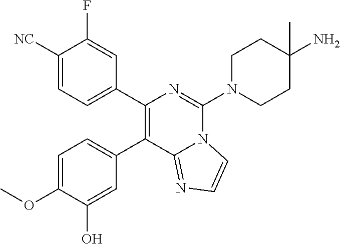 [SMILES: COc1ccc(-c2c(-c3ccc(C#N)c(F)c3)nc(N3CCC(C)(N)CC3)n3ccnc23)cc1O] | ¹H NMR (400 MHz, DMSO-d₆) δ ppm 9.12 (s, 1H), 7.97 − 7.83 (m, 2H), 7.72 (d, J = 1.2 Hz, 1H), 7.58 (dd, J = 11.2, 1.6 Hz, 1H), 7.43 (dd, J = 8.2, 1.6 Hz, 1H), 7.07 − 6.85 (m, 2H), 6.74 (dd, J = 8.4, 2.0 Hz, 1H), 3.87 (s, 3H), 3.60 (q, J = 4.4 Hz, 4H), 1.85 − 1.73 (m, 2H), 1.67 (d, J = 4.6 Hz, 2H), 1.24 (s, 3H). | 473.2 |
| 144 | 4-(5-((3- aminopropyl) (methyl)amino)- 8-(3-hydroxy-4- methoxyphenyl) imidazolo[1,2-c] pyrimidin-7-yl)- 2- fluorobenzonitrile | 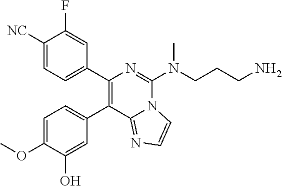 [SMILES: COc1ccc(-c2c(-c3ccc(C#N)c(F)c3)nc(N(C)CCCN)n3ccnc23)cc1O] | ¹H NMR (400 MHz, DMSO-d₆) δ ppm 8.06 (d, J = 1.6 Hz, 1H), 7.80 (dd, J = 8.0, 7.0 Hz, 1H), 7.61 (d, J = 1.4 Hz, 1H), 7.48 (dd, J = 11.0, 1.6 Hz, 1H), 7.34 (dd, J = 8.2, 1.6 Hz, 1H), 6.89 (d, J = 8.4 Hz, 1H), 6.82 (d, J = 2.0 Hz, 1H), 6.64 (dd, J = 8.4, 2.0 Hz, 1H), 3.78 (s, 3H), 3.55 (t, J = 7.4 Hz, 2H), 3.14 (s, 3H), 2.64 (t, J = 6.6 Hz, 2H), 1.82 (dp, J = 14.0, 7.2 Hz, 2H). | 447.2 |
| 145 | 4-(5-(4-amino- 4-methylpiperidin- 1-yl)-8-(3- hydroxy-4- methylphenyl) imidazolo[1,2-c] pyrimidin-7-yl)-2- fluorobenzonitrile | 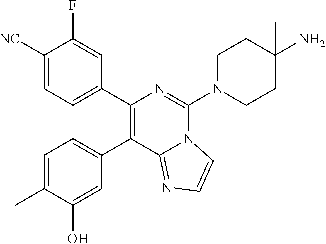 [SMILES: Cc1ccc(-c2c(-c3ccc(C#N)c(F)c3)nc(N3CCC(C)(N)CC3)n3ccnc23)cc1O] | ¹H NMR (400 MHz, DMSO-d₆) δ ppm 9.32 (s, 1H), 7.95 − 7.71 (m, 2H), 7.63 (d, J = 1.2 Hz, 1H), 7.50 (dd, J = 11.0, 1.5 Hz, 1H), 7.34 (dd, J = 8.0, 1.6 Hz, 1H), 7.03 (d, J = 7.6 Hz, 1H), 6.84 (d, J = 1.6 Hz, 1H), 6.59 (dd, J = 7.6, 1.6 Hz, 1H), 3.53 (t, J = 5.4 Hz, 4H), 2.15 (s, 3H), 1.78 − 1.55 (m, 4H), 1.16 (s, 3H). | 457.2 |
| 146 | 4-(5-((3- aminopropyl) (methyl) amino)-8-(3- hydroxy-4- methylphenyl) imidazolo[1,2-c] pyrimidin-7-yl)-2- fluorobenzonitrile | 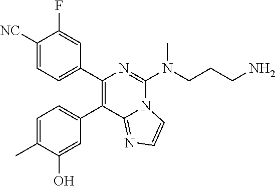 [SMILES: Cc1ccc(-c2c(-c3ccc(C#N)c(F)c3)nc(N(C)CCCN)n3ccnc23)cc1O] | ¹H NMR (400 MHz, DMSO-d₆) δ ppm 9.31 (s, 1H), 8.08 (d, J = 1.6 Hz, 1H), 7.80 (dd, J = 8.2, 7.0 Hz, 1H), 7.61 (d, J = 1.4 Hz, 1H), 7.50 (dd, J = 11.2, 1.6 Hz, 1H), 7.35 (td, J = 7.8, 1.6 Hz, 1H), 7.03 (d, J = 7.8 Hz, 1H), 6.84 (d, J = 1.6 Hz, 1H), 6.59 (dd, J = 7.6, 1.6 Hz, 1H), 3.67 − 3.54 (m, 2H), 3.16 (s, 3H), 2.64 (t, J = 6.4 Hz, 2H), 2.15 (s, 3H), 1.82 (q, J = 7.0 Hz, 2H). | 431.2 |
| 147 | 4-(5-(4-amino-4- methylpiperidin- 1-yl)-8-(4- fluoro-3- hydroxyphenyl) imidazolo[1,2-c] pyrimidin- 7-yl)-2- fluorobenzonitrile | 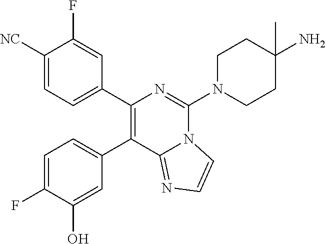 [SMILES: CC1(N)CCN(c2nc(-c3ccc(C#N)c(F)c3)c(-c3ccc(F)c(O)c3)c3nccn23)CC1] | ¹H NMR (400 MHz, DMSO-d₆) δ ppm 7.90 − 7.74 (m, 2H), 7.65 (d, J = 1.2 Hz, 1H), 7.50 (dd, J = 11.0, 1.6 Hz, 1H), 7.31 (dd, J = 8.2, 1.6 Hz, 1H), 7.11 (dd, J = 11.4, 8.2 Hz, 1H), 7.00 (dd, J = 8.6, 2.2 Hz, 1H), 6.67 (ddd, J = 8.4, 4.2, 2.0 Hz, 1H), 3.53 (dd, J = 11.4, 5.6 Hz, 4H), 1.80 − 1.53 (m, 4H), 1.16 (s, 3H). | 461.2 |
| 148 | 4-(5-((3- aminopropyl) (methyl) amino)-8-(4- fluoro-3- hydroxyphenyl) imidazolo [1,2-c]pyrimidin- 7-yl)-2- fluorobenzonitrile | 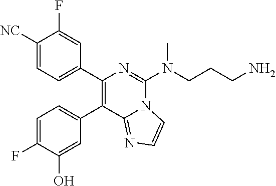 [SMILES: CN(CCCN)c1nc(-c2ccc(C#N)c(F)c2)c(-c2ccc(F)c(O)c2)c2nccn12] | ¹H NMR (400 MHz, DMSO-d₆) δ ppm 8.09 (d, J = 1.6 Hz, 1H), 7.81 (t, J = 7.6 Hz, 1H), 7.61 (d, J = 1.4 Hz, 1H), 7.54 − 7.44 (m, 1H), 7.31 (td, J = 7.8, 1.6 Hz, 1H), 7.10 (dd, J = 11.4, 8.4 Hz, 1H), 6.99 (dd, J = 8.6, 2.2 Hz, 1H), 6.65 (ddd, J = 8.4, 4.4, 2.2 Hz, 1H), 3.57 (t, J = 7.4 Hz, 2H), 3.16 (s, 3H), 2.64 (t, J = 6.4 Hz, 2H), 1.93 − 1.70 (m, J = 7.2 Hz, 2H). | 435.2 |
| 149 | 4-(5-(4-amino-4- methylpiperidin- 1-yl)-8-(5-fluoro- 3-methylbenzo [d]isoxazol-6-yl) imidazolo[1,2-c] pyrimidin-7-yl)-2- fluorobenzonitrile | 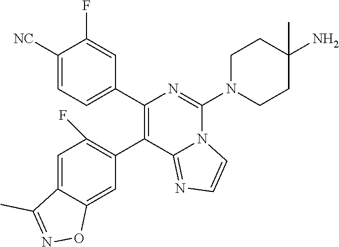 [SMILES: Cc1noc2cc(-c3c(-c4ccc(C#N)c(F)c4)nc(N4CCC(C)(N)CC4)n4ccnc34)c(F)cc12] | ¹H NMR (400 MHz, Chloroform-d) δ 7.62 − 7.55 (m, 2H), 7.48 − 7.41 (m, 2H), 7.31 (dd, J = 8.0, 6.6 Hz, 1H), 7.25 (d, J = 8.0 Hz, 1H), 7.11 (dd, J = 8.0, 1.6 Hz, 1H), 3.74 (s, 2H), 3.58 (d, J = 7.0 Hz, 2H), 2.52 (s, 3H), 1.99 − 1.87 (m, 4H), 1.47 (s, 3H). | 500.2 |
| 150 | 4-(5-(4-amino-4- methylpiperidin- 1-yl)-8-(5-fluoro- 3-methylbenzo [d]isoxazol-6-yl) imidazolo[1,2-c] pyrimidin-7-yl)-2- fluorobenzonitrile | 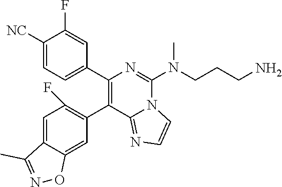 [SMILES: Cc1noc2cc(-c3c(-c4ccc(C#N)c(F)c4)nc(N(C)CCCN)n4ccnc34)c(F)cc12] | ¹H NMR (400 MHz, DMSO-d₆) δ ppm 7.22 (d, J = 1.6 Hz, 1H), 6.91 (d, J = 5.2 Hz, 1H), 6.81 − 6.74 (m, 3H), 6.70 (dd, J = 10.4, 1.6 Hz, 1H), 6.52 (dd, J = 8.2, 1.6 Hz, 1H), 2.95 (t, J = 7.2 Hz, 2H), 2.54 (s, 3H), 2.05 (t, J = 7.2 Hz, 2H), 1.80 (s, 3H), 1.24 (q, J = 7.4 Hz, 2H). | 474.2 |
| 151 | 5-(5-(4- aminopiperidin-1- yl)-7-(3-fluoro-4- methylphenyl) imidazolo[1,2-c] pyrimidin-8-yl)-2- methoxyphenol hydrochloride | 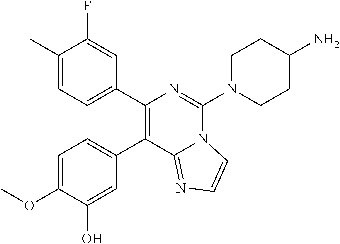 [SMILES: COc1ccc(-c2c(-c3ccc(C)c(F)c3)nc(N3CCC(N)CC3)n3ccnc23)cc1O] | ¹H NMR (400 MHz, DMSO-d₆) δ ppm 9.29 (s, 1H), 8.30 (s, 3H), 8.16 (s, 1H), 8.03 (s, 1H), 7.27 − 7.18 (m, 2H), 7.15 (dd, J = 8.0, 1.6 Hz, 1H), 7.03 (d, J = 8.4 Hz, 1H), 6.78 (d, J = 2.2 Hz, 1H), 6.71 (dd, J = 8.2, 2.2 Hz, 1H), 4.06 (d, J = 13.2 Hz, 2H), 3.82 (s, 3H), 3.40 (d, J = 5.2 Hz, 1H), 3.26 (d, J = 25.0 Hz, 2H), 2.21 (d, J = 1.8 Hz, 3H), 2.14 − 2.04 (m, 2H), 1.87 (qd, J = 12.2, 4.0 Hz, 2H). | 448.2 |
| 152 | 4-(5-(4-amino-4- methylpiperidin- 1-yl)-8-(3- hydroxy-4- (trifluoromethoxy) phenyl)imidazolo [1,2-c]pyrimidin- 7-yl)-2- fluorobenzonitrile | 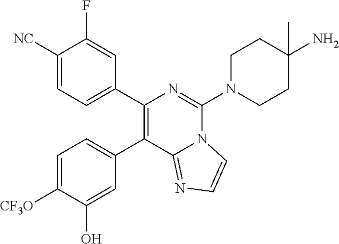 | ¹H NMR (400 MHz, DMSO-d₆) δ ppm 7.92 − 7.78 (m, 2H), 7.49 (d, J = 1.8 Hz, 1H), 7.66 (d, J = 1.4 Hz, 1H), 7.41 − 7.31 (m, 1H), 7.24 (d, J = 8.4 Hz, 1H), 7.06 (d, J = 2.2 Hz, 1H), 6.76 (dd, J = 8.4, 2.2 Hz, 1H), 3.53 (d, J = 18.8 Hz, 4H), 1.84 − 1.56 (m, 4H), 1.19 (s, 3H). | 527.2 |
| 153 | 4-(5-((3- aminopropyl) (methyl) amino)-8-(3- hydroxy-4- (trifluoromethoxy) phenyl)imidazolo [1,2-c]pyrimidin- 7-yl)-2- fluorobenzonitrile | 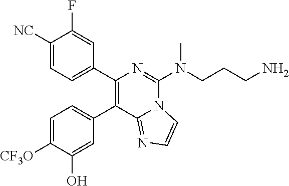 | ¹H NMR (400 MHz, DMSO-d₆) δ ppm 8.10 (d, J = 1.6 Hz, 1H), 7.82 (t, J = 7.6 Hz, 1H), 7.61 (d, J = 1.6 Hz, 1H), 7.45 (dd, J = 11.0, 1.6 Hz, 1H), 7.34 (dd, J = 8.2, 1.6 Hz, 1H), 7.28 − 7.15 (m, 1H), 7.05 (d, J = 2.0 Hz, 1H), 6.73 (dd, J = 8.4, 2.0Hz, 1H), 3.65 − 3.55 (m, 2H), 3.17 (s, 3H), 2.65 (t, J = 6.6 Hz, 2H), 1.81 (p, J = 6.8 Hz, 2H). | 501.2 |
| 154 | 4-(5-(4- aminopiperidin- 1-yl)-8-(3- hydroxy-4- (trifluoromethyl) phenyl)imidazolo [1,2-c]pyrimidin- 7-yl)-2- fluorobenzonitrile carboxylate | 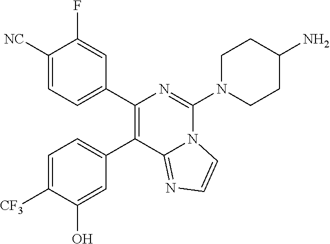 [SMILES: N#Cc1ccc(-c2nc(N3CCC(N)CC3)n3ccnc3c2-c2ccc(C(F)(F)F)c(O)c2)cc1F] | ¹H NMR (400 MHz, DMSO-d₆) δ ppm: 8.32 (s, 1H), 7.92 (d, J = 1.4 Hz, 1H), 7.84 (t, J = 7.6 Hz, 1H), 7.68 (d, J = 1.4 Hz, 1H), 7.56 (dd, J = 10.8, 1.4 Hz, 1H), 7.48 (d, J = 8.2 Hz, 1H), 7.30 (dd, J = 8.2, 1.5 Hz, 1H), 7.11 (s, 1H), 6.82 (d, J = 8.2 Hz, 1H), 4.01 (d, J = 13.2 Hz, 2H), 3.17 (s, 1H), 3.13 (d, J = 12.2 Hz, 2H), 2.00 (d, J = 12.6 Hz, 2H), 1.72 (h, J = 9.0 Hz, 2H). | 497.2 |
| 155 | 4-(5-(3-amino-8- azadicyclo[3.2.1] octane-8-yl)-8-(3- hydroxy-4- methoxyphenyl) imidazolo[1,2-c] pyrimidin-7-yl)-2- fluorobenzonitrile hydrochloride | 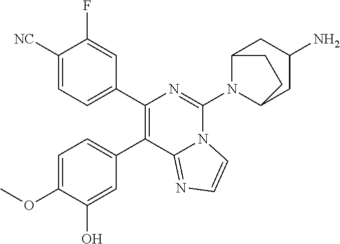 [SMILES: COc1ccc(-c2c(-c3ccc(C#N)c(F)c3)nc(N3C4CCC3CC(N)C4)n3ccnc23)cc1O] | ¹H NMR (400 MHz, DMSO-d₆) δ ppm 9.13 (s, 1H), 8.16(s, 3H), 8.06 (s, 1H), 7.83 (t, J = 7.6 Hz, 2H), 7.53 (d, J = 11.0 Hz, 1H), 7.35 (d, J = 15.0 Hz, 1H), 6.95 (d, J = 8.4 Hz, 1H), 6.81 (d, J = 2.0 Hz, 1H), 6.67 (dd, J = 8.2, 2.0 Hz, 1H), 4.76 (s, 2H), 3.80 (S, 3H), 3.35 (s, 1H), 2.24 − 1.77 (m, 8H). | 485.2 |
| 156 | 4-(5-(3-amino-8- azabicyclo[3.2.1] octane-8-yl)-8- (3-hydroxy-4- methylphenyl) imidazolo[1,2-c] pyrimidin-7-yl)- 2-fluorobenzo nitrile hydrochloride | 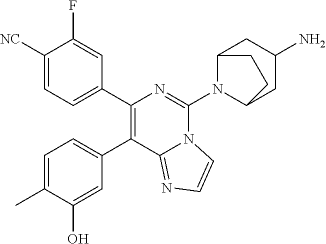 [SMILES: Cc1ccc(-c2c(-c3ccc(C#N)c(F)c3)nc(N3C4CCC3CC(N)C4)n3ccnc23)cc1O] | ¹H NMR (400 MHz, DMSO-d₆) δ ppm 9.60 (s, 1H), 8.18 (s, 4H), 8.01 (s, 1H), 7.85 (t, J = 7.4 Hz, 1H), 7.57 (d, J = 10.8 Hz, 1H), 7.35 (dd, J = 8.0, 1.4 Hz, 1H), 7.14 (d, J = 7.7 Hz, 1H), 6.82 (d, J = 1.6 Hz, 1H), 6.63 (dd, J = 7.6, 1.6 Hz, 1H), 4.84 − 4.72 (m, 2H), 3.62 (s, 1H), 2.27 − 1.79 (m, 11H). | 469.2 |
| 157 | 4-(5-(3-amino- 8-azabicyclo [3.2.1]octane-8- yl)-8-(4-fluoro- 3-hydroxyphenyl) imidazolo [1,2-c]pyrimidin- 7-yl)-2- fluorobenzonitrile hydrochloride | 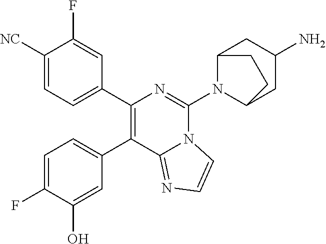 [SMILES: N#Cc1ccc(-c2nc(N3C4CCC3CC(N)C4)n3ccnc3c2-c2ccc(F)c(O)c2)cc1F] | ¹H NMR (400 MHz, DMSO-d₆) δ ppm 10.09 (s, 1H), 8.12 (s, 4H), 8.02 − 7.76 (m, 2H), 7.56 (d, J = 10.6 Hz, 1H), 7.32(d, J = 10.8 Hz, 1H), 7.18 (dd, J = 11.2, 8.4 Hz, 1H), 7.00 (dd, J = 8.6, 2.0 Hz, 1H), 6.69 (ddd, J = 8.4, 4.2, 2.0 Hz, 1H), 4.79 (t, J = 3.6 Hz, 2H), 3.62 (s, 1H), 2.28 − 1.67 (m, 8H). | 473.2 |
| 158 | 4-(5-(3-amino-8- azabicyclo[3.2.1] octane-8-yl)-8- (4-chloro-3- hydroxyphenyl) imidazolo[1,2-c] pyrimidin-7-yl)-2- fluorobenzonitrile hydrochloride | 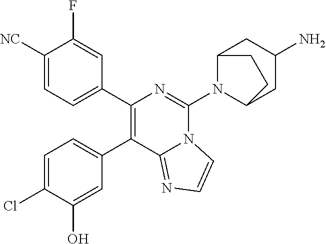 [SMILES: N#Cc1ccc(-c2nc(N3C4CCC3CC(N)C4)n3ccnc3c2-c2ccc(Cl)c(O)c2)cc1F] | ¹H NMR (400 MHz, DMSO-d₆) δ ppm 10.41 (s, 1H), 8.14(s, 3H),8.12 (s, 1H), 7.96 − 7.78 (m, 2H), 7.59 (d, J = 10.8 Hz, 1H), 7.44 − 7.20 (m, 2H), 7.03 (d, J = 1.8 Hz, 1H), 6.72 (dd, J = 8.2, 1.8 Hz, 1H), 4.88 − 4.74 (m, 2H), 3.62 (s, 1H), 2.25 − 1.77 (m, 8H). | 489.2 |
| 159 | 4-(5-(8-amino- 3-azabicyclo[3. 2.1]octane-3-yl)- 8-(3-hydroxy-4- methylphenyl) imidazolo[1,2-c] pyrimidin-7-yl)-2- fluorobenzonitrile | 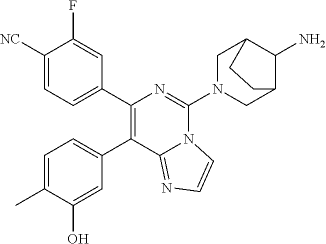 [SMILES: Cc1ccc(-c2c(-c3ccc(C#N)c(F)c3)nc(N3CC4CCC(C3)C4N)n3ccnc23)cc1O] | ¹H NMR (400 MHz, DMSO-d₆) δ ppm 9.32 (s, 1H), 7.92 (d, J = 1.6 Hz, 1H), 7.81 (dd, J = 8.2, 7.0 Hz, 1H), 7.63 (d, J = 1.4 Hz, 1H), 7.50 (dd, J = 11.2, 1.4 Hz, 1H), 7.39 − 7.26 (m, 1H), 7.03 (d, J = 7.8 Hz, 1H), 6.84 (d, J = 1.8 Hz, 1H), 6.59 (dd, J = 7.4, 1.8 Hz, 1H), 3.72 − 3.54 (m, 4H), 3.29 (s, 2H), 3.17 (t, J = 4.6 Hz, 1H), 2.13 (d, J = 12.8 Hz, 3H), 1.86 (d, J = 8.6 Hz, 4H). | 469.2 |
| 160 | 4-(5-(5-amino- 2-azadicyclo[2. 2.1]heptan-2- yl)-8-(3-hydroxy- 4-methylphenyl) imidazolo[1,2-c] pyrimidin-7-yl)-2- fluorobenzonitrile hydrochloride | 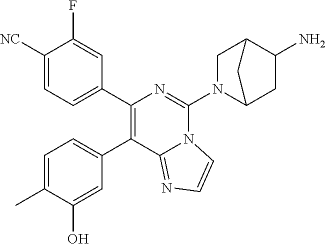 [SMILES: Cc1ccc(-c2c(-c3ccc(C#N)c(F)c3)nc(N3CC4CC3CC4N)n3ccnc23)cc1O] | ¹H NMR (400 MHz, DMSO-d₆) δ ppm 9.62 (s, 1H), 8.54 − 8.13 (m, 4H), 7.96 (d, J = 8.0 Hz, 1H), 7.88 (ddd, J = 8.2, 6.8, 1.4 Hz, 1H), 7.48 (ddd, J = 10.8, 9.4, 1.6 Hz, 1H), 7.34 (dd, J = 8.2, 1.6 Hz, 1H), 7.15 (dd, J = 7.8, 4.0 Hz, 1H), 6.78 (dd, J = 3.4, 1.8 Hz, 1H), 6.60 (dt, J = 7.4, 2.4 Hz, 1H), 4.79 (d, J = 57.2 Hz, 1H), 4.18 (ddd, J = 39.6, 36.6, 9.8 Hz, 2H), 3.61 − 3.41 (m, 2H), 2.93 (m, 1H), 2.30 − 2.11 (m, 4H), 2.07 − 1.80 (m, 2H). | 455.2 |
| 161 | 4-(5-(8-amino- 3-azabicyclo[3. 2.1]octane-3-yl)- 8-(3-hydroxy-4- methoxyphenyl) imidazolo[1,2-c] pyrimidin- 7-yl)-2- fluorobenzonitrile hydrochloride | 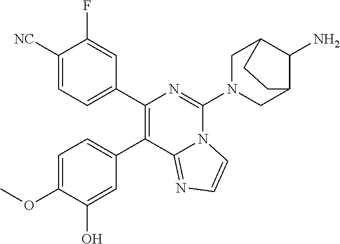 [SMILES: COc1ccc(-c2c(-c3ccc(C#N)c(F)c3)nc(N3CC4CCC(C3)C4N)n3ccnc23)cc1O] | ¹H NMR (400 MHz, DMSO-d6 + D20) δ 8.06 (d, J = 2.1Hz, 1H), 7.93 (d, J = 1.9 Hz, 1H), 7.83 (dd, J = 8.1, 6.9 Hz, 1H), 7.49 (dd, J = 10.8, 1.5 Hz, 1H), 7.36 (dd, J = 8.2, 1.5 Hz, 1H), 6.99 (d, J = 8.3 Hz, 1H), 6.76 (d, J = 2.1Hz, 1H), 6.70 (dd, J = 8.2, 2.1Hz, 1H), 3.86 (dd, J = 13.5, 3.2 Hz, 2H), 3.79 (s, 3H), 3.59 (s, 2H), 3.35 (t, J = 4.6 Hz, 1H), 2.45 (s, 2H), 1.88 (s, 1H), 1.85 (d, J = 4.6 Hz, 3H), 1.83 (s, 1H). | 485.2 |
| 162 | 4-(5-(8-amino- 3-azabicyclo [3.2.1]octyl-3- yl)-8-(3-fluoro-4- methoxyphenyl) imidazolo[1,2-c] pyrimidin-7-yl)-2- fluorobenzonitrile hydrochloride | 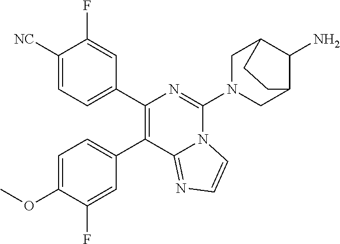 [SMILES: COc1ccc(-c2c(-c3ccc(C#N)c(F)c3)nc(N3CC4CCC(C3)C4N)n3ccnc23)cc1F] | ¹H NMR (400 MHz, Methanol-d₄) δ ppm 8.20 − 8.05 (m, 1H), 8.01 − 7.83 (m, 1H), 7.60 (dd, J = 8.0, 6.6 Hz, 1H), 7.40 (d, J = 10.2 Hz, 1H), 7.29 (d, J = 8.2 Hz, 1H), 7.14 (q, J = 11.2, 9.8 Hz, 2H), 7.03 (d, J = 8.4 Hz, 1H), 4.31 − 4.11 (m, 2H), 3.85 (s, 3H), 3.61 (s, 1H), 3.54 (d, J = 12.4 Hz, 2H), 2.56 (d, J = 4.4 Hz, 2H), 2.04 − 1.80 (m, 4H). | 487.2 |
| 163 | 4-(5-(6-amino-3- azabicyclo[3.1.1] heptan-3-yl)-8- (3-hydroxy-4- methoxyphenyl) imidazolo[1,2-c] pyrimidin- 7-yl)-2- fluorobenzonitrile hydrochloride | 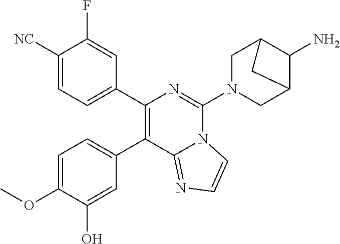 [SMILES: COc1ccc(-c2c(-c3ccc(C#N)c(F)c3)nc(N3CC4CC(C3)C4N)n3ccnc23)cc1O] | ¹H NMR (400 MHz, Methanol-d₄) δ 8.70 (d, J = 2.6 Hz, 1H), 7.86 (d, J = 2.4 Hz, 1H), 7.66 (dd, J = 8.2, 6.8 Hz, 1H), 7.55 (dd, J = 10.6, 1.6 Hz, 1H), 7.45 (dd, J = 8.2, 1.6 Hz, 1H), 7.08 (d, J = 8.2 Hz, 1H), 6.85 − 6.77 (m, 2H), 4.62 (d, J = 12.2 Hz, 2H), 4.40 (d, J = 12.2 Hz, 2H), 3.93 (s, 3H), 3.05 − 2.97 (m, 2H), 2.66 (s, 1H), 2.20 (dt, J = 11.2, 5.8 Hz, 1H), 1.77 (d, J = 10.2 Hz, 1H). | 471.2 |
| 164 | 4-(5-(5-amino- 2-azabicyclo[2.2. 1]heptan-2-yl)-8- (3-hydroxy-4- methoxyphenyl) imidazolo[1,2-c] pyrimidin- 7-yl)-2- fluorobenzonitrile hydrochloride | 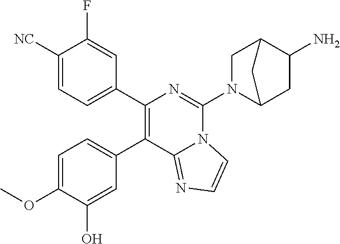 [SMILES: COc1ccc(-c2c(-c3ccc(C#N)c(F)c3)nc(N3CC4CC3CC4N)n3ccnc23)cc1O] | ¹H NMR (400 MHz, Methanol-d₄) δ 8.29 (dd, J = 4.2, 2.4 Hz, 1H), 7.87 (dd, J = 4.4, 2.2 Hz, 1H), 7.66 (ddd, J = 8.2, 6.8, 4.2 Hz, 1H), 7.50 (dd, J = 10.6, 1.4 Hz, 1H), 7.42 (dt, J = 8.2, 1.6 Hz, 1H), 7.06 (dd, J = 8.2, 1.0 Hz, 1H), 6.85 − 6.72 (m, 2H), 4.98 (d, J = 42.6 Hz, 1H), 4.41 − 4.20 (m, 2H), 3.92 (s, 3H), 3.18 − 2.98 (m, 1H), 2.88 − 2.73 (m, 1H), 2.48 − 2.05 (m, 3H), 2.05 − 1.81 (m, 1H). | 471.2 |
| 165 | 4-(5-(6-amino- 3-azabicyclo [3.1.1]heptane- 3-yl)-8-(3- hydroxy-4- methylphenyl) imidazolo[1,2- c]pyrimidin- 7-yl)-2- fluorobenzonitrile | 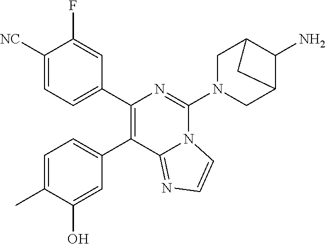 [SMILES: Cc1ccc(-c2c(-c3ccc(C#N)c(F)c3)nc(N3CC4CC(C3)C4N)n3ccnc23)cc1O] | ¹H NMR (400 MHz, DMSO-d₆) δ ppm 9.26 (s, 1H), 8.42 (d, J = 1.6 Hz, 1H), 7.78 (t, J = 7.6 Hz, 1H), 7.61 − 7.26 (m, 3H), 7.14 − 6.47 (m, 3H), 4.41 − 3.97 (m, 4H), 3.31 (s, 1H), 2.14 (s, 3H), 1.74 (dt, J = 11.2, 6.2 Hz, 1H), 1.56 − 1.03 (m, 3H). | 455.2 |
| 166 | 4-(5-(5-amino- 2-azabicyclo [2.2.2]octane- 2-yl)-8-(3- hydroxy-4- methoxyphenyl) imidazolo[1,2-c] pyrimidin-7- yl)-2- fluorobenzonitrile hydrochloride | 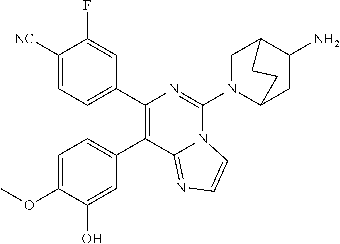 [SMILES: COc1ccc(-c2c(-c3ccc(C#N)c(F)c3)nc(N3CC4CCC3CC4N)n3ccnc23)cc1O] | ¹H NMR (400 MHz, Methanol-d₄) δ 8.26 (d, J = 2.4 Hz, 1H), 7.92 − 7.86 (m, 1H), 7.66 (dd, J = 8.2, 6.7 Hz, 1H), 7.49 (dd, J = 10.6, 1.6 Hz, 1H), 7.42 (dd, J = 8.2, 1.6 Hz, 1H), 7.07 (d, J = 8.2 Hz, 1H), 6.84 − 6.76 (m, 2H), 4.72 − 4.63 (m, 1H), 4.29 (d, J = 11.4 Hz, 1H), 4.14 (dt, J = 11.4, 1.7 Hz, 1H), 3.92 (s, 3H), 3.70 (dd, J = 8.4, 4.4 Hz, 1H), 2.54 − 2.43 (m, 1H), 2.39 − 2.28 (m, 2H), 2.22 − 2.12 (m, 1H), 2.05 (ddd, J = 13.8, 7.8, 3.4 Hz, 1H), 1.97 − 1.82 (m, 2H). | 485.2 |
| 167 | 4-(5-(6-amino- 3-azabicyclo [3.1.1]heptane- 3-yl)-8-(3- fluoro-4- methoxyphenyl) imidazolo[1,2- c]pyrimidin- 7-yl)-2- fluorobenzonitrile hydrochloride | 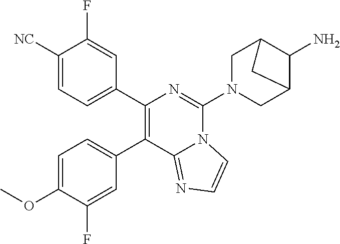 [SMILES: COc1ccc(-c2c(-c3ccc(C#N)c(F)c3)nc(N3CC4CC(C3)C4N)n3ccnc23)cc1F] | ¹H NMR (400 MHz, Methanol-d₄)oppm 8.73 (d, J = 2.4 Hz, 1H), 7.90 (d, J = 2.2 Hz, 1H), 7.69 (dd, J = 8.2, 6.8 Hz, 1H), 7.58 (dd, J = 10.4, 1.4 Hz, 1H), 7.41 (dd, J = 8.2, 1.4 Hz, 1H), 7.35 − 7.17 (m, 2H), 7.17 − 6.98 (m, 1H), 4.64 (d, J = 12.2 Hz, 2H), 4.43 (d, J = 12.2 Hz, 2H), 3.97 (s, 3H), 3.89 (t, J = 5.8 Hz, 1H), 3.03 (t, J = 6.0 Hz, 2H), 2.22 (dt, J = 11.2, 6.0 Hz, 1H), 1.80 (d, J = 10.2 Hz, 1H). | 473.2 |
| 168 | 4-(5-(5-amino- 2-azabicyclo [2.2.2]octane-2- yl)-8-(3-hydroxy- 4-methylphenyl) imidazolo[1,2-c] pyrimidin-7-yl)- 2-fluorobenzo nitrile | 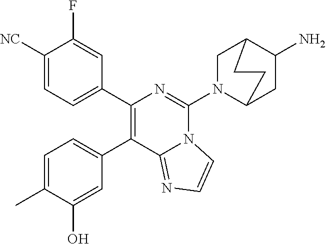 [SMILES: Cc1ccc(-c2c(-c3ccc(C#N)c(F)c3)nc(N3CC4CCC3CC4N)n3ccnc23)cc1O] | ¹H NMR (400 MHz, DMSO-d₆) δ ppm 9.27 (s, 1H), 8.02 (dd, J = 15.8, 1.6 Hz, 1H), 7.78 (t, J = 7.6 Hz, 1H), 7.59 − 7.40 (m, 2H), 7.32 (dd, J = 8.2, 1.4 Hz, 1H), 7.01 (d, J = 7.8 Hz, 1H), 6.86 − 6.38 (m, 2H), 4.38 − 4.20 (m, 2H), 3.78 (dd, J = 30.4, 10.1Hz, 2H), 3.16 (s, 1H), 2.14 (s, 5H), 1.94 − 1.49 (m, 4H). | 469.2 |
| 169 | 4-(5-(5-amino-2- azabicyclo[2.2.1] heptane-2-yl)-8- (3-fluoro-4- methoxyphenyl) imidazolo[1,2- c]pyrimidin- 7-yl)-2- fluorobenzonitrile hydrochloride | 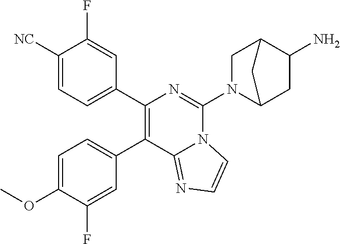 [SMILES: COc1ccc(-c2c(-c3ccc(C#N)c(F)c3)nc(N3CC4CC3CC4N)n3ccnc23)cc1F] | ¹H NMR (400 MHz, Methanol-d₄) δ ppm 8.14 (d, J = 2.2 Hz, 1H), 7.73 (d, J = 2.0 Hz, 1H), 7.51 (t, J = 7.4 Hz, 1H), 7.33 (d, J = 10.4 Hz, 1H), 7.23 (d, J = 8.0 Hz, 1H), 7.13 − 6.99 (m, 2H), 6.94 (d, J = 8.4 Hz, 1H), 4.17 (dd, J = 9.2, 3.8 Hz, 1H), 3.78 (s, 4H), 3.59 − 3.38 (m, 1H), 3.15 (q, J = 1.8 Hz, 1H), 2.89 (d, J = 3.4 Hz, 1H), 2.62 (dd, J = 14.6, 7.6 Hz, 1H), 2.04 (d, J = 11.2 Hz, 1H), 1.97 − 1.82 (m, 1H), 1.72 (d, J = 13.8 Hz, 1H). | 473.2 |
| 170 | 4-(5-(5-amino-2- azabicyclo[2.2.2] octane-2-yl)-8- (3-fluoro-4- methoxyphenyl) imidazolo[1,2-c] pyrimidin-7-yl)-2- fluorobenzonitrile hydrochloride | 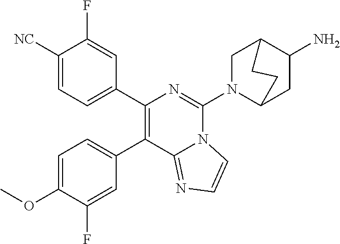 [SMILES: COc1ccc(-c2c(-c3ccc(C#N)c(F)c3)nc(N3CC4CCC3CC4N)n3ccnc23)cc1F] | ¹H NMR (400 MHz, Methanol-d₄) δ ppm 8.23 (d, J = 2.4 Hz, 1H), 7.83 (d, J = 2.4 Hz, 1H), 7.58 (dd, J = 8.2, 6.8 Hz, 1H), 7.39 (dd, J = 10.4, 1.6 Hz, 1H), 7.26 (dd, J = 8.2, 1.4 Hz, 1H), 7.20 − 7.05 (m, 2H), 6.99 (dt, J = 8.4, 1.6 Hz, 1H), 4.65 − 4.50 (m, 1H), 4.31 − 4.00 (m, 2H), 3.84 (s, 3H), 3.70 (d, J = 9.4 Hz, 1H), 2.78 (ddt, J = 13.4, 9.8, 3.4 Hz, 1H), 2.37 − 2.10 (m, 2H), 2.07 − 1.78 (m, 3H), 1.65 (ddd, J = 14.4, 5.4, 1.8 Hz, 1H). | 487.2 |
| 171 | 4-(5-(3-amino-8- azabicyclo[3.2.1] octane-8-yl)-8- (5-fluoroindole- 6-yl)imidazolo [1,2-c]pyrimidin- 7-yl)-2- fluorobenzonitrile | 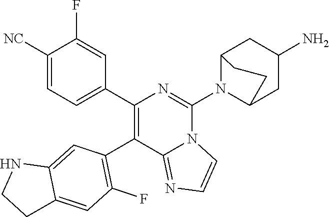 [SMILES: N#Cc1ccc(-c2nc(N3C4CCC3CC(N)C4)n3ccnc3c2-c2cc3c(cc2F)CCN3)cc1F] | ¹H NMR (400 MHz, DMSO-d₆) δ ppm 7.93 (d, J = 1.5 Hz, 1H), 7.84 (dd, J = 8.2, 7.0 Hz, 1H), 7.62 (d, J = 1.4 Hz, 1H), 7.53 (dd, J = 11.0, 1.6 Hz, 1H), 7.41 (dd, J = 8.0, 1.6 Hz, 1H), 6.90 (d, J = 9.4 Hz, 1H), 6.47 (d, J = 5.8 Hz, 1H), 4.72 (s, 2H), 3.44 (t, J = 8.6 Hz, 2H),3.32 (s, 1H), 2.96 (t, J = 8.6 Hz, 2H), 2.19 − 1.65 (m, 8H). | 498.2 |
| 172 | 4-(5-(3-amino-8- azabicyclo[3.2.1] octane-8-yl)-8- (6-fluoro-1H- benzo[d] imidazole-5-yl) imidazolo [1,2-c]pyrimidin- 7-yl)-2- fluorobenzonitrile | 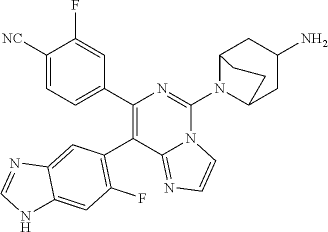 [SMILES: N#Cc1ccc(-c2nc(N3C4CCC3CC(N)C4)n3ccnc3c2-c2cc3nc[nH]c3cc2F)cc1F] | ¹H NMR (400 MHz, DMSO-d₆) δ ppm 8.31 (s, 1H), 7.97 (d, J = 1.5 Hz, 1H), 7.76 (dd, J = 8.2, 7.0 Hz, 1H), 7.64 (d, J = 1.4 Hz, 2H), 7.57 − 7.35 (m, 2H), 7.30 (dd, J = 8.2, 1.6 Hz, 1H), 4.75 (s, 2H), 3.34 (s, 1H), 2.15 − 1.78 (m, 8H). | 497.2 |
| 173 | 4-(5-(3-amino-8- azabicyclo[3.2.1] octane-8-yl)-8-(5- fluoro-1H- indazole-6-yl) imidazolo[1,2-c] pyrimidin- 7-yl)-2- fluorobenzonitrile hydrochloride | 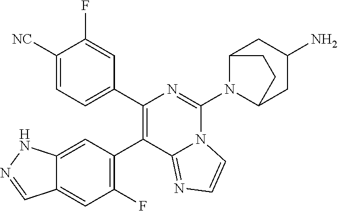 [SMILES: N#Cc1ccc(-c2nc(N3C4CCC3CC(N)C4)n3ccnc3c2-c2cc3[nH]ncc3cc2F)cc1F] | ¹H NMR (400 MHz, Methanol-d₄) δ ppm 8.20 (d, J = 2.4 Hz, 1H), 8.09 (s, 1H), 7.96 (d, J = 2.2 Hz, 1H), 7.67 (d, J = 5.6 Hz, 1H), 7.63 − 7.44 (m, 3H), 7.27 (dd, J = 8.2, 1.6 Hz, 1H), 5.00 − 4.88 (m, 2H), 3.73 (td, J = 10.4, 5.4 Hz, 1H), 2.33 − 2.07 (m, 6H), 1.96 (t, J = 7.4 Hz, 2H). | 497.2 |
| 174 | 4-(5-(3-amino-8- azabicyclo[3.2.1] octane-8-yl)-8- (5-fluoro-1- methyl-1H-indole- 6-yl)imidazolo [1,2-c]pyrimidin- 7-yl)-2-fluoro benzonitrile | 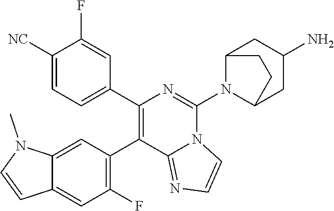 [SMILES: Cn1ccc2cc(F)c(-c3c(-c4ccc(C#N)c(F)c4)nc(N4C5CCC4CC(N)C5)n4ccnc34)cc21] | ¹H NMR (400 MHz, Methanol-d₄) δ ppm 7.89 (s, 1H), 7.61 (d, J = 22.0 Hz, 2H), 7.55 − 7.40 (m, 3H), 7.34 (d, J = 8.2 Hz, 1H), 7.32 − 7.10 (m, 2H), 4.76 (s, 3H), 3.78 (s, 3H), 2.32 − 2.11 (m, 2H), 2.05 (d, J = 12.8 Hz, 2H), 1.88 (dq, J = 30.6, 12.4 Hz, 4H). | 510.2 |
| 175 | 4-(5-(3-amino-8- azabicyclo[3.2.1] octane-8-yl)-8-(6- fluorobenzo[d] [1,3]dioxane-5- -yl)imidazolo [1,2-c]pyrimidin- 7-yl)-2- fluorobenzonitrile hydrochloride | 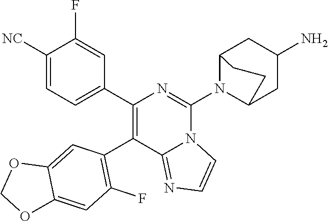 [SMILES: N#Cc1ccc(-c2nc(N3C4CCC3CC(N)C4)n3ccnc3c2-c2cc3c(cc2F)OCO3)cc1F] | ¹H NMR (400 MHz, Methanol-d₄) δ ppm 8.16 (d, J = 2.4 Hz, 1H), 7.98 (d, J = 2.4 Hz, 1H), 7.64 (t, J = 7.4 Hz, 1H), 7.47 (d, J = 10.2 Hz, 1H), 7.34 (d, J = 8.2 Hz, 1H), 6.90 − 6.59 (m, 2H), 6.01 (d, J = 3.4 Hz, 2H), 4.88 (d, J = 6.8 Hz, 2H), 3.72 (p, J = 8.6 Hz, 1H), 2.34 − 1.84 (m, 8H). | 501.2 |
| 176 | 5-(5-(3-amino-8- azabicyclo[3.2.1] octane-8-yl)-8- (3-fluoro-4- methoxyphenyl) imidazolo[1,2-c ] pyrimidin-7-yl) picolinonitrile hydrochloride | 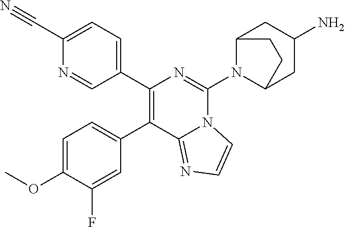 [SMILES: COc1ccc(-c2c(-c3ccc(C#N)nc3)nc(N3C4CCC3CC(N)C4)n3ccnc23)cc1F] | ¹H NMR (400 MHz, Methanol-d₄) δ 8.59 (d, J = 2.2 Hz, 1H), 7.88 (dd, J = 8.2, 2.2 Hz, 1H), 7.80 (d, J = 1.6 Hz, 1H), 7.67 (d, J = 8.2 Hz, 1H), 7.54 (d, J = 1.6 Hz, 1H), 7.10 (dd, J = 12.0, 2.1Hz, 1H), 7.01 (t, J = 8.6 Hz, 1H), 6.91 (dt, J = 8.4, 1.5 Hz, 1H), 4.67 (s, 2H), 3.81 (s, 3H), 3.43 − 3.29 (m, 1H), 2.11 (dd, J = 10.6, 5.3 Hz, 2H), 2.05 − 1.94 (m, 2H), 1.83 (q, J = 8.2 Hz, 4H). | 470.2 |
| 177 | 4-(5-(3-amino-8- azabicyclo[3.2.1] octane-8-yl)-8- (3-fluoro-4- methoxyphenyl) imidazolo[1,2-c] pyrimidin-7-yl)- 1-methyl-1H- pyrrole-2- carbonitrile hydrochloride | 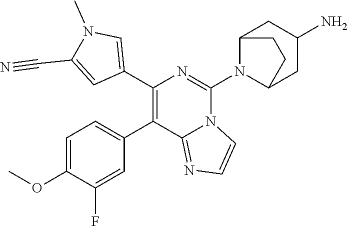 [SMILES: COc1ccc(-c2c(-c3cc(C#N)n(C)c3)nc(N3C4CCC3CC(N)C4)n3ccnc23)cc1F] | ¹H NMR (400 MHz, Methanol-d₄) δ 7.64 (d, J = 1.6 Hz, 1H), 7.36 (d, J = 1.6 Hz, 1H), 7.14 (t, J = 8.6 Hz, 1H), 7.04 − 6.96 (m, 2H), 6.88 (d, J = 1.8 Hz, 1H), 6.38 (d, J = 1.8 Hz, 1H), 4.59 (d, J = 3.8 Hz, 2H), 3.87 (s, 3H), 3.60 (s, 3H), 3.22 (s, 1H), 2.14 − 2.06 (m, 2H), 1.95 (ddd, J = 13.2, 5.7, 2.9 Hz, 2H), 1.88 − 1.68 (m, 4H). | 472.2 |
| 178 | 4-(5-(3-amino- 8-azabicyclo [3.2.1]octane-8- yl)-8-(3-fluoro-4- methoxyphenyl) imidazolo[1,2-c] pyrimidin-7-yl) benzonitrile hydrochloride | 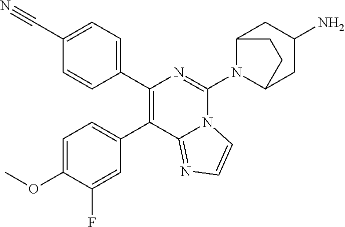 [SMILES: COc1ccc(-c2c(-c3ccc(C#N)cc3)nc(N3C4CCC3CC(N)C4)n3ccnc23)cc1F] | ¹H NMR (400 MHz, Methanol-d₄) δ 7.76 (d, J = 1.6 Hz, 1H), 7.58 − 7.41 (m, 5H), 7.03 (dd, J = 12.0, 2.1Hz, 1H), 6.97 (t, J = 8.6 Hz, 1H), 6.88 (ddd, J = 8.4, 2.1, 1.1Hz, 1H), 4.62 (t, J = 3.8 Hz, 2H), 3.79 (s, 3H), 3.32 − 3.22 (m, 1H), 2.15 − 2.04 (m, 2H), 2.01 − 1.90 (m, 2H), 1.86 − 1.70 (m, 4H). | 469.2 |
| 179 | 4-(5-(3-amino- 8-azadicyclo [3.2.1]octane-8- yl)-8-(3-fluoro-4- methoxyphenyl) imidazolo[1,2-c] pyrimidin-7-yl) cyclohexane-1- carbonitrile hydrochloride | 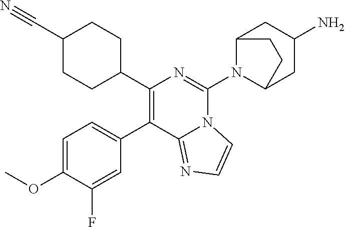 [SMILES: COc1ccc(-c2c(C3CCC(C#N)CC3)nc(N3C4CCC3CC(N)C4)n3ccnc23)cc1F] | ¹H NMR (400 MHz, Methanol-d₄) δ 7.64 (d, J = 1.6 Hz, 1H), 7.36 (d, J = 1.6 Hz, 1H), 7.11 (td, J = 8.8, 4.2 Hz, 1H), 7.05 − 6.95 (m, 2H), 4.55 (d, J = 4.0 Hz, 2H), 3.84 (s, 3H), 3.34 − 3.22 (m, 1H), 2.96 (t, J = 3.6 Hz, 1H), 2.69 − 2.47 (m, 1H), 2.15 − 2.00 (m, 4H), 2.00 − 1.85 (m,4H), 1.85 − 1.65 (m, 4H), 1.66 − 1.34 (m, 4H) | 475.3 |
| 180 | 5-(5-(3-amino- 8-azabicyclo [3.2.1]octane-8- yl)-8-(3-fluoro-4- methoxyphenyl) imidazolo[1,2-c] pyrimidin-7-yl) pyrimidin-2- carbonitrile | 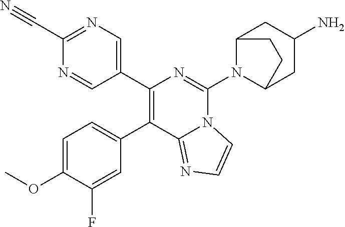 [SMILES: COc1ccc(-c2c(-c3cnc(C#N)nc3)nc(N3C4CCC3CC(N)C4)n3ccnc23)cc1F] | ¹H NMR (400 MHz, DMSO-d₆) δ 8.96 (s, 2H), 8.03 (d, J = 1.6 Hz, 1H), 7.76 (d, J = 1.4 Hz, 1H), 7.43 (dd, J = 12.4, 2.1Hz, 1H), 7.23 (t, J = 8.8 Hz, 1H), 7.18 − 7.13 (m, 1H), 4.76 (t, J = 3.8 Hz, 2H), 3.94 (s, 3H), 3.20 (tt, J = 11.2, 5.5 Hz, 1H), 2.11 (dd, J = 8.6, 4.3 Hz, 2H), 1.95 (ddd, J = 13.2, 5.5, 2.8 Hz, 2H), 1.87 (t, J = 6.8 Hz, 2H), 1.78 − 1.62 (m, 2H). | 471.3 |
| 181 | 4-(5-(3-amino- 8-azabicyclo [3.2.1] octane-8-yl)-8- (3-fluoro-4- methoxyphenyl) imidazolo[1,2- c]pyrimidin-7-yl) phthalonitrile hydrochloride | 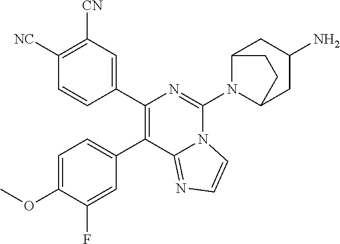 [SMILES: COc1ccc(-c2c(-c3ccc(C#N)c(C#N)c3)nc(N3C4CCC3CC(N)C4)n3ccnc23)cc1F] | ¹H NMR (400 MHz, DMSO-d₆) δ 8.19 (br, 3H), 8.15 (s, 1H), 8.05 (d, J = 8.2 Hz, 1H), 7.95 (s, 1H), 7.77 (d, J = 8.2 Hz, 1H), 7.37 − 7.28 (m, 1H), 7.24 − 7.19 (m, 1H), 7.14 (d, J = 29.6 Hz, 1H), 7.01 (d, J = 8.4 Hz, 1H), 4.85 − 4.78 (m, 2H), 3.88 (s, 3H), 3.74 (s, 1H), 2.15 − 2.11 (m, 2H), 2.07 (d, J = 9.0 Hz, 4H), 1.87 (t, J = 7.0 Hz, 2H). | 494.2 |
| 182 | 2-(5-(5-(3-amino- 8-azadicyclo [3.2.1]octane-8- yl)-8-(3-fluoro-4- methoxyphenyl) imidazolo[1,2-c] pyrimidin-7-yl)-2- fluorophenyl) acetonitrile hydrochloride | 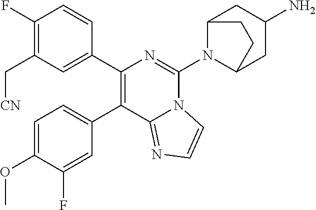 [SMILES: COc1ccc(-c2c(-c3ccc(F)c(CC#N)c3)nc(N3C4CCC3CC(N)C4)n3ccnc23)cc1F] | ¹H NMR (400 MHz, Methanol-d₄) δ ppm 8.19 (d, J = 2.4 Hz, 1H), 7.98 (d, J = 2.2 Hz, 1H), 7.70 (dd, J = 7.4, 2.2 Hz, 1H), 7.51 (ddd, J = 8.6, 5.0, 2.3 Hz, 1H), 7.29 − 7.03 (m, 4H), 4.93 (t, J = 3.6 Hz, 2H), 3.94 (s, 3H), 3.90 (s, 2H), 3.81 (t, J = 8.6 Hz, 1H), 2.32 (dd, J = 8.8, 4.4 Hz, 2H), 2.22 (dd, J = 8.8, 2.8 Hz, 4H), 2.07 − 1.94 (m, 2H). | 501.2 |
| 183 | 4-(5-(3-amino-8- azabicyclo[3.2.1] octane-8-yl)-8-(3- fluoro-4- methoxyphenyl) imidazolo[1,2-c] pyrimidin-7-yl)- 2,3-difluorobe nzonitrile hydrochloride | 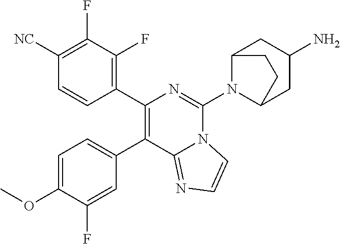 [SMILES: COc1ccc(-c2c(-c3ccc(C#N)c(F)c3F)nc(N3C4CCC3CC(N)C4)n3ccnc23)cc1F] | ¹H NMR (400 MHz, DMSO-d₆) § 8.36 (s, 3H), 7.99 (d, J = 1.6 Hz, 1H), 7.80 (ddd, J = 7.8, 5.8, 1.6 Hz, 1H), 7.72 (d, J = 1.4 Hz, 1H), 7.52 (t, J = 7.0 Hz, 1H), 7.30 (dd, J = 12.6, 2.2 Hz, 1H), 7.08 (t, J = 8.8 Hz, 1H), 6.96 (dt, J = 8.6, 1.6 Hz, 1H), 4.66 (d, J = 4.6 Hz, 2H), 3.84 (s, 3H), 3.51 (s, 1H), 2.06 (dd, J = 8.6, 4.2 Hz, 2H), 1.95 (s, 2H), 1.82 (dd, J = 17.2, 11.0 Hz, 4H). | 505.2 |
| 184 | 4-(5-(3-amino-8- azabicyclo[3.2.1] octane-8-yl)-8- (3-fluoro-4- methoxyphenyl) imidazolo[1,2-c] pyrimidin-7-yl)-2- methylbenzonitrile hydrochloride | 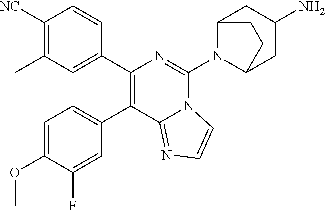 [SMILES: COc1ccc(-c2c(-c3ccc(C#N)c(C)c3)nc(N3C4CCC3CC(N)C4)n3ccnc23)cc1F] | ¹H NMR (400 MHz, Methanol-d₄) δ ppm 8.12 (d, J = 2.2 Hz, 1H), 7.91 (d, J = 1.8 Hz, 1H), 7.59-7.40 (m, 2H), 7.29 (d, J = 8.0 Hz, 1H), 7.23-7.06 (m, 2H), 7.00 (d, J = 8.4 Hz, 1H), 4.83 (s, 2H), 3.84 (s, 3H), 3.69 (q, J = 11.0, 9.8 Hz, 1H), 2.36 (s, 3H), 2.29- 2.19 (m, 2H), 2.19− 2.05 (m, 4H), 1.92 (t, J = 7.0 Hz, 2H). | 483.2 |
| 185 | 4-(5-(3-amino-8- azabicyclo[3.2.1] octane-8-yl)-8- (3-fluoro-4- methoxyphenyl) imidazolo[1,2-c] pyrimidin-7-yl)-2- hydroxybenzonitrile | 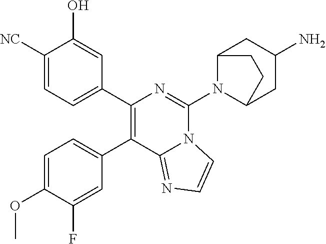 [SMILES: COc1ccc(-c2c(-c3ccc(C#N)c(O)c3)nc(N3C4CCC3CC(N)C4)n3ccnc23)cc1F] | ¹H NMR (400 MHz, Methanol-d₄) δ ppm 8.10 (d, J = 2.2 Hz, 1H), 7.91 (d, J = 2.2 Hz, 1H), 7.36 (d, J = 8.2 Hz, 1H), 7.21 − 7.06 (m, 2H), 7.06 − 6.94 (m, 2H), 6.90 (dd, J = 8.2, 1.4 Hz, 1H), 4.81 (d, J = 5.8 Hz, 2H), 3.84 (s, 3H), 3.70 (dt, J = 17.2, 9.8 Hz, 1H), 2.21 (dd, J = 8.0, 3.8 Hz, 2H), 2.11 (dd, J = 9.2, 4.4 Hz, 4H), 1.96 − 1.79 (m, 2H). | 485.2 |
| 186 | 4-(5-(3-amino-8- azabicyclo[3.2.1] octane-8-yl)-8-(6- fluorobenzo[c] [1,2,5]oxadiazole- 5-yl)imidazolo [1,2-c]pyrimidin- 7-yl)-2- fluorobenzonitrile | 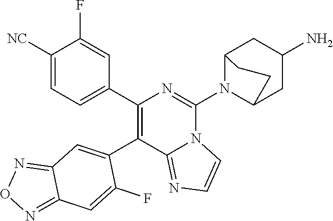 [SMILES: N#Cc1ccc(-c2nc(N3C4CCC3CC(N)C4)n3ccnc3c2-c2cc3nonc3cc2F)cc1F] | ¹H NMR (400 MHz, DMSO-d₆) δ ppm 8.32 (d, J = 6.4 Hz, 1H), 8.16 − 7.96 (m, 2H), 7.81 (dd, J = 8.0, 6.9 Hz, 1H), 7.77 − 7.60 (m, 2H), 7.38 (dd, J = 8.0, 1.6 Hz, 1H), 4.81 (d, J = 4.2 Hz, 2H), 3.18 (dq, J = (dd, J = 36.0, 9.8 Hz, 4H), 1.67 (q, J = 11.2, 5.6 Hz, 1H), 2.13 − 2.01 (m, 2H), 1.88 11.0 Hz, 2H). | 499.2 |

### Example 187

### Preparation of 4-(5-(4-aminopiperidin-1-yl)-8-(3-hydroxy-4-methoxyphenyl)-3-methylimidazolo[1,2-c]pyrimid in-7-yl)-2-fluorobenzonitrile

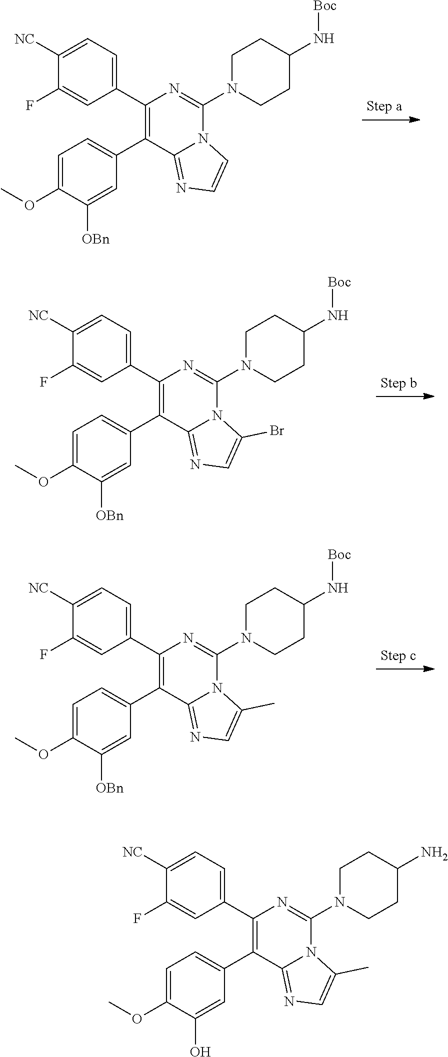 [SMILES: COc1ccc(-c2c(-c3ccc(C#N)c(F)c3)nc(N3CCC(NC(=O)OC(C)(C)C)CC3)n3ccnc23)cc1OCc1ccccc1 ; COc1ccc(-c2c(-c3ccc(C#N)c(F)c3)nc(N3CCC(NC(=O)OC(C)(C)C)CC3)n3c(Br)cnc23)cc1OCc1ccccc1 ; COc1ccc(-c2c(-c3ccc(C#N)c(F)c3)nc(N3CCC(NC(=O)OC(C)(C)C)CC3)n3c(C)cnc23)cc1OCc1ccccc1 ; COc1ccc(-c2c(-c3ccc(C#N)c(F)c3)nc(N3CCC(N)CC3)n3c(C)cnc23)cc1O]

### Step a): Synthesis of tert-butyl(1-(8-(3-(benzyloxy)-4-methoxyphenyl)-3-bromo-7-(4-cyano-3-fluorophenyl)imidazolo [1,2-c]pyrimidin-5-yl)piperidin-4-yl) carbamate

Tert-butyl(1-(8-(3-(benzyloxy)-4-methoxyphenyl)-7-(4-cyano-3-fluorophenyl)imidazolo[1,2-c]p yrimidin-5-yl)piperidin-4-yl) carbamate (100 mg, 0.15 mmol) is dissolved in acetonitrile (3 mL), cooled to 0° C. in an ice water bath, protected with nitrogen, added with NBS (27.5 mg, 0.15 mmol), and continue to react for 30 min while stirring. When LC-MS shows that the reaction is completed, the reaction solution is added with water (3 mL), extracted with ethyl acetate (10 mL×3), washed with saline (10 mL), dried with anhydrous sodium sulfate, filtered, and concentrated; and the residue is purified by silica gel chromatography (eluent: petroleum ether/ethyl acetate=1/1) to obtain tert-butyl(1-(8-(3-(benzyloxy)-4-methoxyphenyl)-3-bromo-7-(4-cyano-3-fluorophenyl)imidazolo [1,2-c]pyrimidin-5-yl)piperidin-4-yl) carbamate with a yield of 94.6%.

¹H NMR (400 MHz, DMSO-d₆) δ ppm 7.73 (ddd, J=8.2, 7.0, 5.2 Hz, 1H), 7.66-7.64 (m, 2H), 7.60 (s, 1H), 7.56 (ddd, J=7.2, 3.2, 1.0 Hz, 5H), 7.39 (d, J=1.2 Hz, 1H), 7.33 (d, J=4.0 Hz, 1H), 7.00 (d, J=8.4 Hz, 1H), 6.95 (t, J=1.8 Hz, 1H), 4.93 (d, J=2.2 Hz, 2H), 3.81 (s, 3H), 3.54 (d, J=12.8 Hz, 2H), 3.15-3.05 (m, 1H), 2.72 (s, 2H), 1.90 (s, 2H), 1.75-1.64 (m, 2H), 1.40 (d, J=1.4 Hz, 9H).

ESI-MS m/z: 727.2[M+H]⁺.

### Step b): Synthesis of tert-butyl(1-(8-(3-(benzyloxy)-4-methoxyphenyl)-7-(4-cyano-3-fluorophenyl)-3-methylimidazol o[1,2-c]pyrimidin-5-yl)piperidin-4-yl-carbamate

Tert-butyl(1-(8-(3-(benzyloxy)-4-methoxyphenyl)-3-bromo-7-(4-cyano-3-fluorophenyl)imidazol o[1,2-c]pyrimidin-5-yl)piperidin-4-yl)carbamate (106 mg, 0.15 mmol), 2,4,6-methyl-1,3,5,2,4,6-trioxatetraborane (339 mg, 2.68 mmol), potassium carbonate (74 mg, 0.54 mmol), and tetrakis(triphenylphosphine)palladium (42 mg, 0.04 mmol) are dissolved with 1,4-dioxane (10 mL); since the mixture is warmed to 100° C. for 1 h, when the LC-MS shows that the reaction is completed, it is transferred to a rotary evaporator and concentrated to dryness; and the residue is purified by silica gel chromatography (petroleum ether/ethyl acetate=1/2) to obtain tert-butyl(1-(8-(3-(benzyloxy)-4-methoxyphenyl)-7-(4-cyano-3-fluorophenyl)-3-methylimidazol o[1,2-c]pyrimidin-5-yl)piperidin-4-ylcarbamate with a yield of 94.0%.

ESI-MS m/z: 663.3[M+H]⁺.

### Step c): Synthesis of 4-(5-(4-aminopiperidin-1-yl)-8-(3-hydroxy-4-methoxyphenyl)-3-methylimidazolo[1,2-c]pyrimid in-7-yl)-2-fluorobenzonitrile

Tert-butyl(1-(8-(3-(benzyloxy)-4-methoxyphenyl)-7-(4-cyano-3-fluorophenyl)-3-methylimidazol o[1,2-c]pyrimidin-5-yl)piperidin-4-yl-carbamate (90 mg, 0.14 mmol) is added with trifluoroacetic acid (3 mL) and dissolved in a sealed tube, and under the nitrogen protection, warmed to 70° C. and reacted for 2 h; when the reaction is completed, the system is concentrated to dryness to obtain a crude product; and the crude product is purified by a pre-HPLC preparation column to obtain 4-(5-(4-aminopiperidin-1-yl)-8-(3-hydroxy-4-methoxyphenyl)-3-methylimidazolo[1,2-c]pyrimid in-7-yl)-2-fluorobenzonitrile with a yield of 18.3%.

¹H NMR (400 MHz, DMSO-d₆) δ ppm 9.02 (s, 1H), 7.79 (dd, J=8.2, 7.0 Hz, 1H), 7.47 (dd, J=11.2, 1.6 Hz, 1H), 7.39 (d, J=1.2 Hz, 1H), 7.32 (dd, J=8.2, 1.6 Hz, 1H), 6.90 (d, J=8.4 Hz, 1H), 6.82 (d, J=2.2 Hz, 1H), 6.65 (dd, J=8.4, 2.2 Hz, 1H), 3.79 (s, 3H), 3.55 (d, J=12.6 Hz, 2H), 2.94 (s, 3H), 2.73 (s, 3H), 1.91 (d, J=12.4 Hz, 2H), 1.61 (t, J=11.4 Hz, 2H).

ESI-MS m/z: 473.2[M+H]⁺.

Compounds in Examples 188-194 are prepared according to the synthesis method of Example 3 (separation method for the compounds: free alkali, hydrochloride and formate are separately prepared according to separation methods 4, 1 and 3, respectively), and their structures and characterization data are as follows:

| | | | | MS |
|---|---|---|---|---|
| Examples | Chemical name | Structure | ¹H NMR | (M + H)⁺ |
| 188 | 4-(1-(3-aminoazetidin-1- yl)-6-(p-tolyl)pyrrolo[1,2- a]pyrazin-7-yl)benzonitrile hydrochloride | 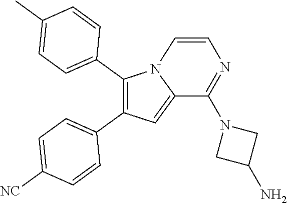 [SMILES: Cc1ccc(-c2c(-c3ccc(C#N)cc3)cc3c(N4CC(N)C4)nccn23)cc1] | ¹H NMR (400 MHz, Methanol-d₄) δ ppm 7.68-7.62 (m, 3H), 7.52-7.48 (m, 2H), 7.43-7.38 (m, 3H), 7.30-7.25 (m, 2H), 6.93 (d, J = 6.0 Hz, 1H), 4.52 (dq, J = 7.8 Hz, 1H), 3.33-3.31 (m, 2H), 3.30- 3.29 (m, 2H), 2.45 (s, 3H). | 380.2 |
| 189 | (R)-4-(1-(3-(methylamino) piperidin-1-yl)-6-(p-tolyl) pyrrolo[1,2-a]pyrazin-7- yl)benzonitrile hydrochloride | 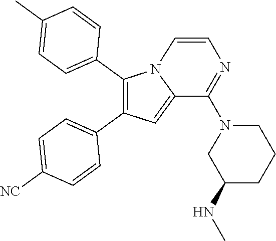 [SMILES: CN[C@@H]1CCCN(c2nccn3c(-c4ccc(C)cc4)c(-c4ccc(C#N)cc4)cc23)C1] | ¹H NMR (400 MHz, Methanol-d₄) δ ppm 7.91 (s, 1H), 7.68-7.63 (m, 2H), 7.58 (dd, J = 5.8, 0.8 Hz, 1H), 7.56- 7.52 (m, 2H), 7.44-7.39 (m, 2H), 7.31-7.26 (m, 2H), 7.03 (d, J = 5.8 Hz, 1H), 4.72 (dd, J = 13.2, 3.8 Hz, 1H), 4.28-4.20 (m, 1H), 3.78 (dd, J = 13.2, 9.8 Hz, 1H), 3.33-3.32 (m , 2H), 3.31-3.29 (m, 2H), 2.84 (s, 3H), 2.46 (s, 3H), 2.18 (dt, J = 12.8, 4.2 Hz, 1H), 2.05-1.84 (m, 1H). | 422.2 |
| 190 | 4-(1-(4-methylpiperazin- 1-yl)-6-(p-tolyl)pyrrolo [1,2-a]pyrazin-7-yl) benzonitrile | 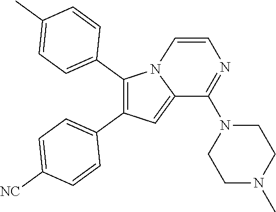 | ¹H NMR (400 MHz, DMSO-d₆) δ ppm 7.75-7.71 (m, 2H), 7.51- 7.47 (m, 2H), 7.35 (d, J = 7.8 Hz, 2H), 7.29-7.24 (m, 3H), 7.23 (d, J = 0.8 Hz, 1H), 7.10 (d, J = 4.8 Hz, 1H), 3.66 (t, J = 4.8 Hz, 4H), 2.51 (s, 2H) , 2.47 (s, 2H), 2.40 (s, 3H), 2.25 (s, 3H). | 408.2 |
| 191 | 4-(1-(piperidin-1-yl)-6-(p- tolyl)pyrrolo[1,2-a] pyrazin-7-yl)benzonitrile | 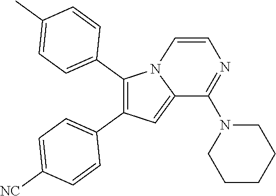 [SMILES: Cc1ccc(-c2c(-c3ccc(C#N)cc3)cc3c(N4CCCCC4)nccn23)cc1] | ¹H NMR (400 MHz, DMSO-d₆) δ ppm 7.75-7.70 (m, 2H), 7.50- 7.45 (m, 2H), 7.35 (d, J = 7.8 Hz, 2H), 7.27-7.22 (m, 3H), 7.15 (d, J = 0.8 Hz, 1H), 7.09 (d, J = 4.8 Hz, 1H), 3.65 (t, J = 4.2 Hz, 4H), 2.39 (s, 3H), 1.67 (s, 6H). | 393.2 |
| 192 | 4-(1-(3-aminopyrrolidin- 1-yl)-6-(p-tolyl)pyrrolo [1,2-a]pyrazine-7-yl) benzonitrile hydrochloride | 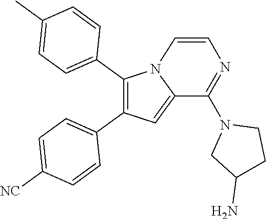 [SMILES: Cc1ccc(-c2c(-c3ccc(C#N)cc3)cc3c(N4CCC(N)C4)nccn23)cc1] | ¹H NMR (400 MHz, DMSO-d₆) δ ppm 8.62 (s, 3H), 7.93 (s, 1H), 7.80 (d, J = 8.2 Hz, 2H), 7.53- 7.46 (m, 2H), 7.42 (d, J = 7.8 Hz, 2H), 7.33-7.20 (m, 3H), 6.96 (d, J = 5.8 Hz, 1H), 4.12 (s, 2H), 2.51 (s, 2H), 2.49 (s, 2H), 2.42 (s, 3H), 2.37 (s, 1H). | 394.2 |
| 193 | 4-(1-(4-aminopiperidin-1- yl)-6-(p-tolyl)pyrrolo[1,2- a]pyrazin-7-yl)benzonitrile hydrochloride | 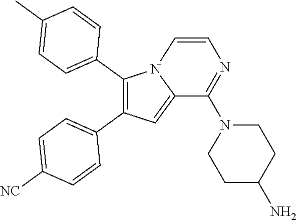 [SMILES: Cc1ccc(-c2c(-c3ccc(C#N)cc3)cc3c(N4CCC(N)CC4)nccn23)cc1] | ¹H NMR (400 MHz, DMSO-d₆) δ ppm 8.37 (s, 3H), 7.86 (m, 1H) 7.81-7.77 (m, 2H), 7.53-7.48 (m, 2H), 7.41 (d, J = 7.8 Hz, 2H), 7.35 (d, J = 5.6 Hz, 1H), 7.29 (d, J = 8.0 Hz, 2H), 7.01 (dd, J = 5.8, 2.2 Hz, 1H), 4.52 (s, 2H), 3.61 (s, 1H), 2.49 (s, 2H), 2.42 (s, 3H), 2.17 (d, J = 12.4 Hz, 2H), 1.82 (d, J = 12.6 Hz, 2H). | 408.2 |
| 194 | 4-(1-(3-amino-8-azabicyclo [3.2.1]octane-8-yl)-6-(5- fluoro-3-methylbenzo[d] isoxazol-6-yl) pyrrolo[1,2-a]pyrazin-7-yl) benzonitrile hydrochloride | 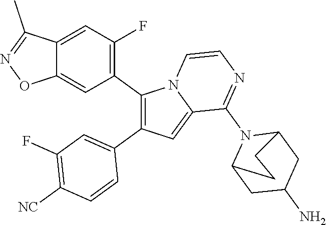 [SMILES: Cc1noc2cc(-c3c(-c4ccc(C#N)c(F)c4)cc4c(N5C6CCC5CC(N)C6)nccn34)c(F)cc12] | ¹H NMR (400 MHz, DMSO-d₆) δ ppm 8.20-8.14 (m, 3H), 8.10 (s, 2H), 8.09-8.00 (m, 2H), 7.99-7.88 (m, 2H), 7.29 (s, 1H), 5.30 (s, 2H), 3.78-3.74 (m, 1H), 2.64 (s, 3H), 2.19- 2.17 (m, 4H), 2.02-1.93 (m, 4H). | 511.2 |

### Example 195

### 4-(1-(4-methylpiperazin-1-yl)-6-(p-tolyl)pyrrolo[1,2-d][1,2,4]triazine-7-yl)benzonitrile is prepared according to the synthesis method (separation method 4) of Example 4, and the structure and representation data are as follows

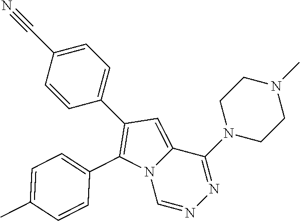

¹H NMR (400 MHz, Chloroform-d) δ ppm 8.57 (s, 1H), 7.55 (d, J=8.0 Hz, 2H), 7.38 (d, J=8.0 Hz, 2H), 7.30 (d, J=7.8 Hz, 2H), 7.21 (d, J=7.8 Hz, 2H), 6.90 (s, 1H), 3.88 (t, J=4.8 Hz, 4H), 2.72-2.61 (m, 4H), 2.44 (s, 3H), 2.40 (s, 3H).

ESI-MS m/z=409.2 [M+H]+.

### Example 196

### 4-(1-(4-aminopiperidin-1-yl)-6-(p-tolyl)pyrrolo[1,2-d][1,2,4]triazin-7-yl)benzonitrile hydrochloride is prepared according to the synthesis method (separation method 4) of Example 4, and the structure and representation data are as follows

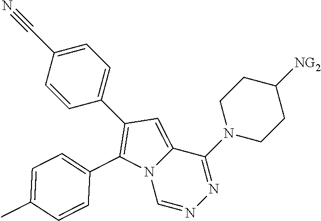

¹H NMR (400 MHz, Chloroform-d) δ ppm 8.58 (s, 3H), 8.48 (d, J=3.2 Hz, 2H), 7.69 (d, J=7.8 Hz, 2H), 7.51 (d, J=8.2 Hz, 2H), 7.39 (d, J=7.8 Hz, 2H), 7.32 (s, 2H), 4.59 (d, J=13.6 Hz, 2H), 3.70 (t, J=12.8 Hz, 2H), 3.57 (s, 1H), 2.46 (s, 3H), 2.33 (d, J=13.2 Hz, 2H), 2.03 (s, 2H).

ESI-MS m/z=409.2 [M+H]⁺.

### Example 197

### Preparation of 4-(4-(3-(methylamino)piperidin-1-yl)-7-(p-tolyl)pyrrolo[1,2-b]pyridazin-6-yl]benzonitrile hydrochloride

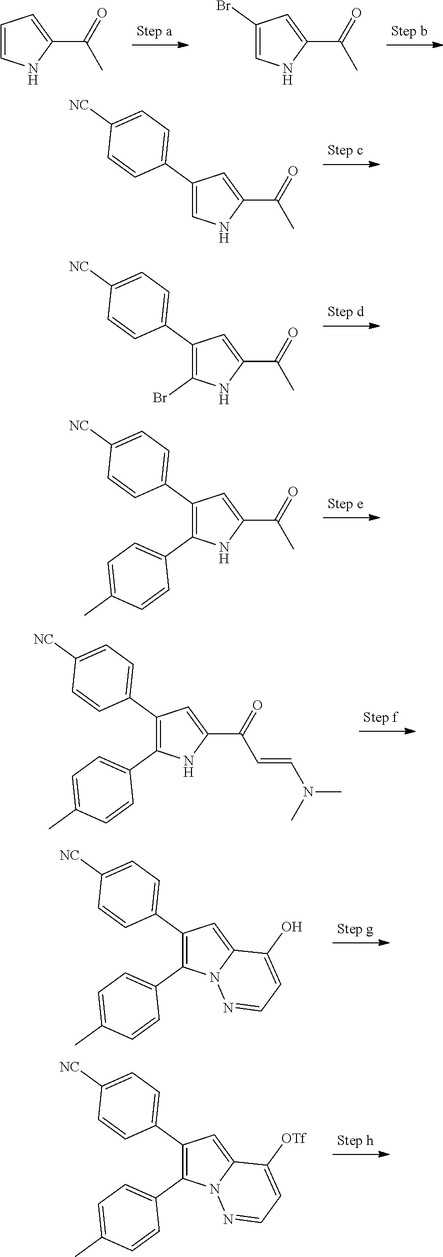

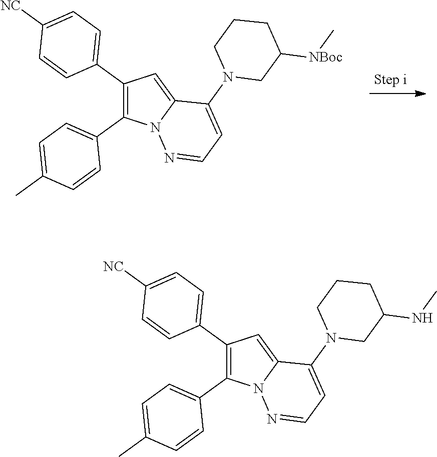

Step a): Preparation of 1-(4-bromo-1H-pyrrol-2-yl)ethane-1-one Acylpyrrole (10.0 g, 91.7 mmol), resin Amberlyst15 (0.9 g, 0.09 g/1.0 g raw material) and anhydrous tetrahydrofuran (150 mL) are sequentially added into a reaction flask, cooled to −30° C. in a cold bath, and slowly added with N-bromosuccinimide (16.3 g, 91.7 mmol), and continue to react for 2 h; when TLC shows that the raw materials disappear, the reaction solution is quenched with a saturated sodium sulfite solution (20 mL), and extracted with dichloromethane (100 mL×3); the organic phase is combined, washed with saturated saline (40 mL), dried with anhydrous sodium sulfate, filtered, and concentrated; and the residue is purified by silica gel chromatography (eluent: petroleum ether/ethyl acetate=6/1) to obtain 1-(4-bromo-1H-pyrrol-2-yl)ethane-1-one with a yield of 91.0%.

ESI-MS m/z: 188.9[M+H]⁺.

### Step b): Preparation of 4-(5-acetyl-1H-pyrrol-3-yl)benzonitrile

1-(4-bromo-1H-pyrrol-2-yl)ethane-1-one (1.0 g, 5.35 mmol), p-cyanophenylboronic acid (1.96 g, 13.37 mmol), Pd(dppf)Cl₂ (1.17 g, 1.60 mmol), cesium carbonate (6.09 g, 18.7 mmol), 1,4-dioxane (10 mL) and water (2 mL) are sequentially added into a reaction flask, and under nitrogen protection, reacted in microwaves at 100° C. for 60 min. When LC-MS shows that raw materials disappear, a product is generated. The reaction solution is concentrated under vacuum, dissolved in ethyl acetate (10 mL), and washed with water (10 mL); aqueous phase is extracted with ethyl acetate (10 mL×3); organic phase is combined, washed with saturated saline (10 mL), dried with anhydrous sodium sulfate, filtered, and concentrated; and the residue is purified by silica gel chromatography (eluent: petroleum ether/ethyl acetate=1/1) to obtain 4-(5-acetyl-1H-pyrrol-3-yl)benzonitrile with a yield of 71.4%

¹H NMR (400 MHz, Chloroform-d) δ ppm 9.74 (s, 1H), 7.63 (q, J=8.2 Hz, 4H), 7.42-7.37 (m, 1H), 7.19 (t, J=2.0 Hz, 1H), 2.50 (s, 3H).

ESI-MS m/z: 211.1[M+H]⁺.

### Step c): Preparation of 4-(5-acetyl-2-bromo-1H-pyrrol-3-yl)benzonitrile

4-(5-acetyl-1H-pyrrol-3-yl)benzonitrile (510 mg, 2.43 mmol), resin Amberlyst15 (46 mg, 0.09 g/1.0 g raw material) and anhydrous tetrahydrofuran (60 mL) are sequentially added into a reaction flask, cooled to −30° C. in a cold bath, and slowly added with NBS (432 mg, 2.43 mmol), and continue to react for 2 h; when TLC shows that the raw materials disappear, the reaction solution is quenched with a saturated sodium sulfite solution (10 mL), and extracted with ethyl acetate for three times (10 mL×3); the organic phase is combined, washed with saturated saline (10 mL), dried with anhydrous sodium sulfate, filtered, and concentrated; and the residue is purified by silica gel chromatography (eluent: petroleum ether/ethyl acetate=3/1) to obtain 4-(5-acetyl-2-bromo-1H-pyrrol-3-yl)benzonitrile with a yield of 65.9%.

¹H NMR (400 MHz, Chloroform-d) δ ppm 9.53 (s, 1H), 7.70 (s, 4H), 7.02 (d, J=2.8 Hz, 1H), 2.46 (s, 3H).

ESI-MS m/z: 290.0[M+H]⁺.

### Step d): Preparation of 4-(5-acetyl-2-(p-tolyl)-1H-pyrrol-3-yl)benzonitrile

4-(5-acetyl-2-bromo-1H-pyrrol-3-yl)benzonitrile (460 mg, 1.60 mmol), p-cyanophenylboronic acid (434 mg, 3.20 mmol), Pd(dppf)Cl₂ (117 mg, 0.16 mmol), cesium carbonate (1.56 g, 4.80 mmol), 1,4-dioxane (10 mL) and water (1 mL) are sequentially added into a reaction flask, and under nitrogen protection, reacted in mocrowaves at 100° C. for 60 min. When LC-MS shows that raw materials disappear, a product is generated. The reaction solution is concentrated under vacuum, dissolved in ethyl acetate (10 mL), and washed with water (10 mL); aqueous phase is extracted with ethyl acetate (10 mL×3); and organic phase is combined, then washed with saturated saline (10 mL), dried with anhydrous sodium sulfate, filtered, and concentrated. The residue is purified by silica gel chromatography (eluent: petroleum ether/ethyl acetate=4: 1) to obtain 4-(5-acetyl-2-(p-tolyl)-1H-pyrrol-3-yl)benzonitrile with a yield of 62.5%.

¹HNMR (400 MHz, Chloroform-d) δ ppm 9.39 (s, 1H), 7.60-7.52 (m, 2H), 7.40 (dd, J=8.4, 2.0 Hz, 2H), 7.23 (d, J=7.8 Hz, 2H), 7.18 (d, J=7.8 Hz, 2H), 7.05 (d, J=2.6 Hz, 1H), 2.48 (d, J=2.2 Hz, 3H), 2.38 (s, 3H).

ESI-MS m/z: 301.1[M+H]⁺.

### Step e): Preparation of (E)-4-(5-(3-(dimethylamino)acryloyl)-2-(p-tolyl)-1H-pyrrol-3-yl)benzonitrile

4-(5-acetyl-2-(p-tolyl)-1H-pyrrol-3-yl)benzonitrile (300 mg, 0.99 mmol) is dehydrated with toluene, dissolved in N,N-dimethylformamide dimethyl acetal (15 mL), and reacted at 80° C. for 16 h to form a yellow solid. When TLC shows that the raw materials disappear, a reaction solution is suction-filtered to obtain a filter cake; and the filter cake is washed with petroleum ether and dried to obtain (E)-4-(5-(3-(dimethylamino)acryloyl)-2-(p-tolyl)-1H-pyrrol-3-yl)benzonitrile crude product with a yield of 96.2%, which is directly used in the next step.

ESI-MS m/z: 356.2[M+H]⁺.

### Step f): Preparation of 4-(4-hydroxy-7-(p-tolyl)pyrrolo[1,2-b]pyrazin-6-yl]benzonitrile

(E)-4-(5-(3-(dimethylamino)acryloyl)-2-(p-tolyl)-1-H-pyrrol-3-yl)benzonitrile crude product (345 mg, 0.97 mmol) is dissolved in N-methylpyrrolidone (5 mL), then added with potassium tert-butoxide (163 mg, 1.45 mmol), stirred for 30 min, added with O—(4-nitrobenzoyl)hydroxyamine (353 mg, 1.94 mmol), and reacted at 30° C. for 2 h. LC-MS detection shows that the reaction is completed. The reaction solution is quenched with saturated ammonium chloride (1 mL), added with dilute hydrochloric acid (1 mL) to precipitate solid, and then extracted with ethyl acetate (5 mL×3), and there is no residue in the aqueous phase. After concentration, the residue is purified by silica gel chromatography (eluent: petroleum ether/ethyl acetate=2: 1) to obtain 4-(4-hydroxy-7-(p-tolyl)pyrrolo[1,2-b]pyrazin-6-yl]benzonitrile with a yield of 69.5%.

¹H NMR (400 MHz, DMSO-d₆) δ ppm 11.65 (s, 1H), 7.92 (d, J=5.2 Hz, 1H), 7.73 (d, J=8.0 Hz, 2H), 7.47 (d, J=8.0 Hz, 2H), 7.34-7.20 (m, 4H), 6.95 (s, 1H), 6.10 (d, J=5.4 Hz, 1H), 2.36 (s, 3H).

ESI-MS m/z: 326.1[M+H]⁺.

### Step g): Preparation of 6-(4-cyanophenyl)-7-(p-tolyl)pyrrolo[1,2-b]pyrazine-4-trifluoromethanesulfonate

4-(4-hydroxy-7-(p-tolyl)pyrrolo[1,2-b]pyrazin-6-yl]benzonitrile (120 mg, 0.36 mmol) is dehydrated with toluene, dissolved with dichloromethane (5 mL), added with triethylamine (55 mg, 0.46 mmol), and cooled to −30° C. The solution is added dropwise with 0.5 mL of dichloromethane solution of trifluoromethanesulfonic anhydride (126 mg, 0.44 mmol) and continues to react for 30 min. TLC detection shows that the reaction is completed. The reaction solution is quenched with ice water (10 mL), and extracted with ethyl acetate (5 mL×3); organic phase is combined, washed with saturated saline (5 mL), dried with anhydrous sodium sulfate, filtered and concentrated to obtain a crude product of 6-(4-cyanophenyl)-7-(p-tolyl)pyrrolo[1,2-b]pyrazine-4-trifluoromethane sulfonate with a yield of 94.8%, which is directly used in the next step.

ESI-MS m/: 458.1[M+H]⁺.

### Step h): Preparation of tert-butyl(1-(6-(4-cyanophenyl)-7-(p-tolyl)pyrrolo[1,2-b]pyridazin-4-yl)piperidin-3-yl)carbamate

6-(4-cyanophenyl)-7-(p-tolyl)pyrrolo[1,2-b]pyridazine-4-trifluoromethane sulfonate (156 mg, 0.31 mmol), triethylamine (62 mg, 0.62 mmol, 85ul), tert-butyl methyl(piperidin-3-yl)carbamate (132 mg, 0.62 mmol) are dissolved in N-methylpyrrolidone (5 mL), and reacted at 100° C. for 2 h. LC-MS shows that the product has no change and the reaction is completed. The system is quenched with water (5 mL), and then extracted with ethyl acetate (5 mL×3). Organic phase is combined, washed with saturated saline (5 mL), dried with anhydrous sodium sulfate, filtered, and concentrated to obtain a crude product; and the crude product is purified by a pre-HPLC preparation column (separation method 4), and then freeze-dried with acetonitrile and ultrapure water to obtain tert-butyl(1-(6-(4-cyanophenyl)-7-(p-tolyl)pyrrolo[1,2-b]pyridazin-4-yl)piperidin-3-yl)carbamate with a yield of 26%.

ESI-MS m/: 522.3[M+H]⁺.

### Step i): Preparation of 4-(4-(3-(methylamino)piperidin-1-yl)-7-(p-tolyl)pyrrolo[1,2-b]pyridazin-6-yl]benzonitrile hydrochloride

### Tert-butyl

(1-(6-(4-cyanophenyl)-7-(p-toly)pyrrolo[1,2-b]pyridazin-4-yl)piperidin-3-yl)carbamate (38 mg, 0.07 mmol) is dissolved in a dioxane hydrochloride solution (4 M, 2 mL), stirred under nitrogen protection and reacted for 30 min at room temperature till LC-MS shows that raw materials disappear and the reaction is completed. The reaction system is concentrated to dryness at low temperature, and freeze-dried with acetonitrile and water (5 mL) to obtain 4-(4-(3-(methylamino)piperidin-1-yl)-7-(p-tolyl)pyrrolo[1,2-b]pyrazin-6-yl]benzonitrile hydrochloride with a yield of 47.4%.

¹H NMR (400 MHz, DMSO-d₆) δ ppm 9.27-9.12 (m, 1H), 9.01-8.88 (m, 1H), 7.98-7.91 (m, 1H), 7.79-7.70 (m, 2H), 7.57-7.50 (m, 2H), 7.26 (q, J=8.0 Hz, 4H), 7.04 (s, 1H), 6.17 (d, J=5.4 Hz, 1H), 4.19 (d, J=12.4 Hz, 1H), 3.72 (d, J=12.6 Hz, 1H), 3.11 (q, J=10.8, 10.4 Hz, 2H), 2.63 (d, J=5.6 Hz, 3H), 2.36 (s, 3H), 2.17 (d, J=10.6 Hz, 1H), 1.95 (d, J=13.2 Hz, 1H), 1.68 (t, J=8.8 Hz, 2H), 1.23 (s, 1H).

ESI-MS m/z:422.2[M+H]⁺.

### Example 198

### 4-(4-(4-aminopiperidin-1-yl)-7-(p-tolyl)pyrrolo[1,2-b]pyrazin-6-yl)benzonitrile hydrochloride is prepared according to the synthesis method (separation method 1) of Example 197, and the structure and representation data are as follows

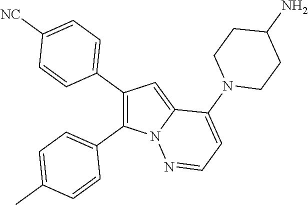 [SMILES: Cc1ccc(-c2c(-c3ccc(C#N)cc3)cc3c(N4CCC(N)CC4)ccnn23)cc1]

¹H NMR (400 MHz, DMSO-d₆) δ ppm 8.24-8.12 (m, 3H), 7.91 (d, J=5.4 Hz, 1H), 7.77-7.71 (m, 2H), 7.55-7.47 (m, 2H), 7.25 (q, J=8.0 Hz, 4H), 6.99 (s, 1H), 6.10 (d, J=5.6 Hz, 1H), 4.08 (d, J=13.0 Hz, 2H), 3.33 (d, J=9.4 Hz, 1H), 3.06 (t, J=12.6 Hz, 2H), 2.36 (s, 3H), 2.07 (d, J=12.4 Hz, 2H), 1.75 (d, J=1.8 Hz, 2H).

ESI-MS m/z:408.2[M+H]⁺.

Compounds in Examples 199-218 are prepared according to the synthesis method of Example 9 (separation method for the compounds: free alkali, hydrochloride and formate are separately prepared according to separation methods 4, 1 and 3, respectively), and their structures and characterization data are as follows:

| | | | | MS |
|---|---|---|---|---|
| Examples | Chemical name | Structure | ¹H NMR | (M + H)⁺ |
| 199 | 4-(5-(3-amino-8-azadicyclo [3.2.1]octane-8-carbonyl)- 2-methylthiophene- 3-yl)-2-fluorobenzonitrile hydrochloride | 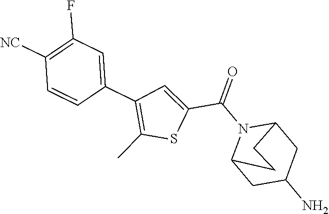 [SMILES: Cc1sc(C(=O)N2C3CCC2CC(N)C3)cc1-c1ccc(C#N)c(F)c1] | ¹H NMR (400 MHz, DMSO-d₆) δ ppm 8.17-7.94 (m, 4H), 7.75 (d, J = 10.8 Hz, 1H), 7.66-7.53 (m, 2H), 4.72 (s, 2H), 3.60 (s, 1H), 2.55 (s, 3H), 1.99 (s, 4H), 1.73 (q, J = 11.8, 9.8 Hz, 4H), 1.34-1.12 (m, 1H). | 370.1 |
| 200 | 4-(5-(3-amino-8-azabicyclo [3.2.1]octane-8-carbonyl)- 2-(1-hydro-indol-6- yl)thiophen-3-yl)-2- fluorobenzonitrile | 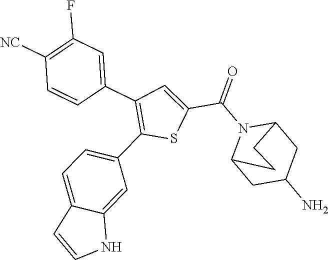 [SMILES: N#Cc1ccc(-c2cc(C(=O)N3C4CCC3CC(N)C4)sc2-c2ccc3cc[nH]c3c2)cc1F] | ¹H NMR (400 MHz, DMSO-d₆) δ ppm 11.23 (s, 1H), 7.82 (t, J = 7.6 Hz, 1H), 7.70-7.51 (m, 3H), 7.43 (s, 1H), 7.36 (s, 1H), 7.32-7.17 (m, 1H), 6.90 (d, J = 8.0 Hz, 1H), 6.46 (s, 1H), 4.68 (s, 2H), 3.13 (s, 1H), 1.80 (d, J = 44.0 Hz, 6H), 1.41 (d, J = 12.6 Hz, 2H). | 471.2 |
| 201 | 4-(5-(3-amino-8-azabicyclo [3.2.1]octane-8-carbonyl)- 2-(pyridin-2-yl)thiophen- 3-yl)-2-fluorobenzonitrile Hydrochloride | 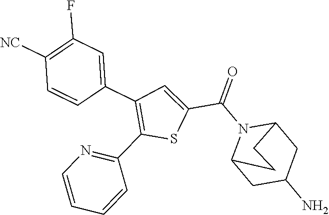 [SMILES: N#Cc1ccc(-c2cc(C(=O)N3C4CCC3CC(N)C4)sc2-c2ccccn2)cc1F] | H NMR (400 MHz, DMSO-d₆) δ ppm 8.62 (d, J = 4.4 Hz, 1H), 8.17 (s, 3H), 7.96 (t, J = 7.8 Hz, 1H), 7.72 (q, J = 10.6, 9.4 Hz, 2H), 7.61 (s, 1H), 7.39 (d, J = 8.0 Hz, 2H), 7.17 (d, J = 8.2 Hz, 1H), 4.76 (s, 2H), 3.61 (s, 1H), 2.04 (d, J = 13.0 Hz, 4H), 1.79 (d, J = 10.8 Hz, 4H). | 433.1 |
| 202 | 4-(5-(3-amino-8-azabicyclo [3.2.1]octane-8-carbonyl)- 2-(pyrimidin-4-yl) thiophen-3-yl)-2-fluoro- benzonitrile hydrochloride | 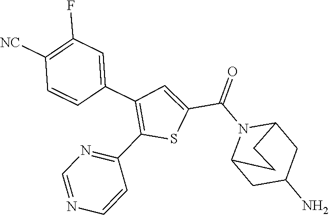 [SMILES: N#Cc1ccc(-c2cc(C(=O)N3C4CCC3CC(N)C4)sc2-c2ccncn2)cc1F] | ¹H NMR (400 MHz, DMSO-d₆) δ ppm 9.20 (d, J = 3.0 Hz, 1H), 8.20 (s, 3H), 8.68 (t, J = 4.4 Hz, 1H), 8.04- 7.98 (m, 1H), 7.92-7.69 (m, 1H), 7.64 (d, J = 3.2 Hz, 1H), 7.57-7.41 (m, 1H), 7.16 (s, 1H) , 4.74 (s, 2H), 3.62 (s, 1H) , 2.02 (d, J = 13.0 Hz, 4H), 1.78 (d, J = 10.4 Hz, 4H). | 434.1 |
| 203 | 4-(5-(3-amino-8-azabicyclo [3.2.1]octane-8-carbonyl)- 2-(1H-indazol-6-yl) thiophen-3-yl)-2-fluoro- benzonitrile | 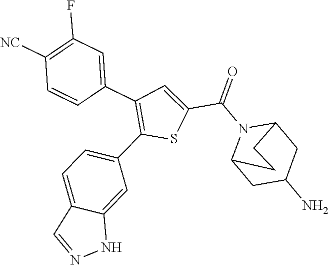 [SMILES: N#Cc1ccc(-c2cc(C(=O)N3C4CCC3CC(N)C4)sc2-c2ccc3cn[nH]c3c2)cc1F] | ¹H NMR (400 MHz, DMSO-d₆) δ ppm 8.12 (d, J = 1.0 Hz, 1H), 7.83 (dd, J = 8.2, 7.2 Hz, 1H), 7.77 (dd, J = 8.4, 0.8 Hz, 1H), 7.69 (s, 1H), 7.62 (dd, J = 10.8, 1.6 Hz, 1H), 7.51 (q, J = 1.2 Hz, 1H), 7.24 (dd, J = 8.2, 1.6 Hz, 1H), 6.98 (dd, J = 8.4, 1.6 Hz, 1H), 4.68 (s, 2H), 3.13 (tt, J = 11.0, 5.6 Hz, 1H), 2.11-1.52 (m, 8H). | 472.2 |
| 204 | (3-amino-8-azabicyclo [3.2.1]octane-8-yl) (5-(5-fluoro-3-methylbenzo [d]isoxazol-6-yl)-4- (pyridin-4-yl)thiophen-2- yl)methanone hydrochloride | 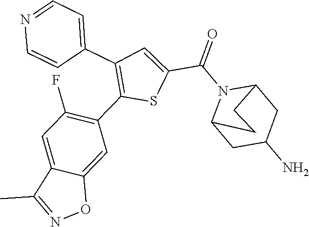 [SMILES: Cc1noc2cc(-c3sc(C(=O)N4C5CCC4CC(N)C5)cc3-c3ccncc3)c(F)cc12] | ¹H NMR (400 MHz, DMSO-d₆) δ ppm 8.74-8.56 (m, 2H), 8.18 (s, 3H), 8.02 (d, J = 5.6 Hz, 1H), 7.93 (s, 1H), 7.87 (d, J = 9.0 Hz, 1H), 7.68-7.47 (m, 2H), 4.80 (s, 2H), 3.79 (s, 1H),2.57 (s, 3H), 2.03 (m, 4H), 1.80 (m, 4H). | 463.2 |
| 205 | (3-amino-8-azabicyclo [3.2.1]octane-8-yl) (5-(5-fluoro-3- methylbenzo[d]isoxazol- 6-yl)-4-(1H-indazol-6-yl) thiophen-2-yl)methanone hydrochloride | 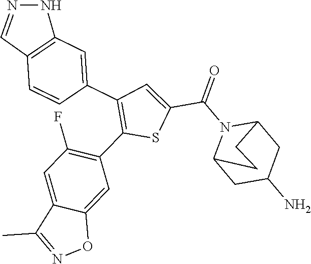 [SMILES: Cc1noc2cc(-c3sc(C(=O)N4C5CCC4CC(N)C5)cc3-c3ccc4cn[nH]c4c3)c(F)cc12] | ¹H NMR (400 MHz, DMSO-d₆) δ ppm 12.99 (s, 1H),8.20 (s, 3H), 7.97 (s, 1H), 7.88 (d, J = 5.4 Hz, 1H), 7.77- 7.67 (m, 2H), 7.62 (d, J = 8.4 Hz, 1H), 7.34 (s, 1H), 6.96 (dd, J = 8.4, 1.4 Hz, 1H), 4.74 (s, 2H), 3.69 (s, 1H), 2.47 (s, 3H), 2.12-1.90 (m, 4H), 1.75 (t, J = 11.8 Hz, 4H). | 502.2 |
| 206 | (3-amino-8-azabicyclo [3.2.1]octan-8-yl)(5-(5- fluoro-3-methylbenzo[d] isoxazol-6-yl)-4- (pyrimidine-5-yl) thiophen-2-yl) methanone hydrochloride | 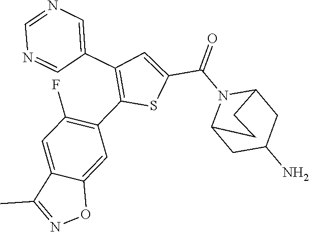 [SMILES: Cc1noc2cc(-c3sc(C(=O)N4C5CCC4CC(N)C5)cc3-c3cncnc3)c(F)cc12] | ¹H NMR (400 MHz, DMSO-d₆) δ ppm 9.12 (s, 1H), 8.75 (s, 2H),8.15 (s, 3H), 7.97 (d, J = 5.4 Hz, 2H), 7.85 (d, J = 9.2 Hz, 1H), 4.78 (s, 2H),3.72 (s, 1H), 2.55 (s, 3H), 1.99 (dt, J = 12.4, 6.8 Hz, 4H), 1.78 (d, J = 12.0 Hz, 4H). | 464.2 |
| 207 | (3-amino-8-azabicyclo [3.2.1]octan-8-yl)(5-(5- fluoro-3-methylbenzo[d] isoxazol-6-yl)-4-phenyithio- phene-2-yl)methanone hydrochloride | 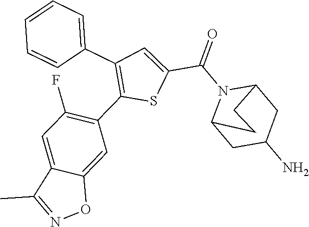 [SMILES: Cc1noc2cc(-c3sc(C(=O)N4C5CCC4CC(N)C5)cc3-c3ccccc3)c(F)cc12] | ¹H NMR (400 MHz, DMSO-d₆) δ ppm 8.18 (s, 3H), 7.87 (d, J = 5.4 Hz, 1H), 7.79 (d, J = 8.8 Hz, 1H), 7.72 (s, 1H), 7.29 (p, J = 5.8 Hz, 5H), 4.77 (s, 2H), 3.61 (dq, J = 12.1, 6.4, 6.0 Hz, 1H), 2.54 (s, 3H), 2.03 (s, 4H), 1.87- 1.68 (m, 4H). | 462.2 |
| 208 | (3-amino-8-azabicyclo [3.2.1]octan-8-yl) (5-(5-fluoro-3-methylbenzo [d]isoxazol-6-yl)-4- (pyridin-3-yl)thiophen-2-yl) methanone hydrochloride | 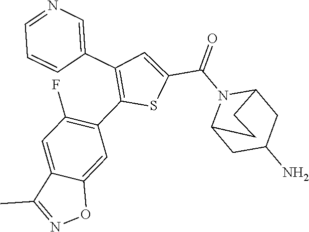 [SMILES: Cc1noc2cc(-c3sc(C(=O)N4C5CCC4CC(N)C5)cc3-c3cccnc3)c(F)cc12] | ¹H NMR (400 MHz, DMSO-d₆) δ ppm 8.80 (d, J = 7.8 Hz, 1H), 8.68 (ddd, J = 15.6, 5.4, 1.6 Hz, 1H),8.16 (s, 3H), 8.09 (s, 3H), 7.88- 7.82 (m, 1H), 7.73-7.61 (m, 1H), 4.78 (s, 2H), 3.63 (s, 1H), 2.55 (s, 3H), 2.04 (s, 4H), 1.89-1.70 (m, 4H). | 463.2 |
| 209 | 4-(5-(3-amino-8-azabicyclo [3.2.1]octan-8-carbonyl)- 2-(6-fluoro-3-methyl benzo[d]isoxazol-7-yl) thiophen-3-yl)-2-fluoro- benzonitrile hydrochloride | 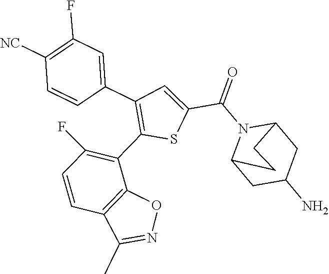 [SMILES: Cc1noc2c(-c3sc(C(=O)N4C5CCC4CC(N)C5)cc3-c3ccc(C#N)c(F)c3)c(F)ccc12] | ¹H NMR (400 MHz, DMSO-d₆) δ ppm 8.06-8.00 (m, 1H), 7.94 (s, 3H), 7.90 (d, J = 2.4 Hz, 1H), 7.81 (ddd, J = 9.6, 7.2, 2.2 Hz, 1H), 7.64 (dd, J = 10.6, 1.6 Hz, 1H), 7.41 (ddd, J = 11.0, 8.8, 2.4 Hz, 1H), 7.20 (dt, J = 8.2, 1.8 Hz, 1H), 4.80 (s, 2H), 3.61 (dt, J = 11.4, 5.6 Hz, 1H), 2.53 (s, 3H), 2.02 (s, 4H), 1.87-1.64 (m, 4H). | 505.1 |
| 210 | (3-amino-8-azabicyclo [3.2.1]octan-8-yl)(5-(5- fluoro-3-methylbenzo[d] isoxazol-6-yl)-4- (pyridin-2-yl)thiophen-2- yl)methanone | 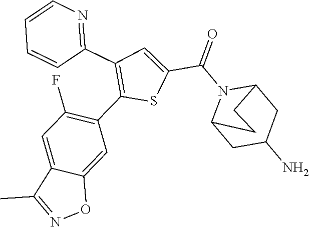 [SMILES: Cc1noc2cc(-c3sc(C(=O)N4C5CCC4CC(N)C5)cc3-c3ccccn3)c(F)cc12] | ¹H NMR (400 MHz, DMSO-d₆) δ ppm 8.46 (dd, J = 4.8, 1.6 Hz, 1H), 8.04- 7.88 (m, 2H), 7.88-7.66 (m, 2H), 7.41 (d, J = 7.8 Hz, 1H), 7.28 (dd, J = 7.6, 4.8 Hz, 1H), 4.68 (s, 2H), 3.18 (dt, J = 11.0, 5.6 Hz, 1H), 2.56 (s, 3H), 2.21-1.39 (m, 8H). | 463.2 |
| 211 | (3-amino-8-azabicyclo [3.2.1]octan-8-yl)(5-(5- fluoro-3-methylbenzo[d] isoxazol-6-yl)-4- (pyridin-2-yl)thiophen-2- yl)methanone | 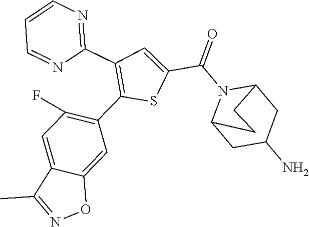 [SMILES: Cc1noc2cc(-c3sc(C(=O)N4C5CCC4CC(N)C5)cc3-c3ncccn3)c(F)cc12] | ¹H NMR (400 MHz, Chloroform-d) δ ppm 8.51 (d, J = 4.8 Hz, 2H), 8.03 (s, 1H), 7.56 (d, J = 5.2 Hz, 1H), 7.15 (d, J = 8.4 Hz, 1H), 7.03 (t, J = 4.8 Hz, 1H), 4.78 (s, 2H), 3.39 (s, 1H), 2.51 (s, 3H), 2.01 (m, 4H), 1.74 (d, J = 9.6 Hz, 4H). | 464.2 |
| 212 | 4-(5-(3-amino-8-azabicyclo [3.2.1]octane-8-carbonyl)- 2-(5-fluoro-3-methyl- benzo[d]isoxazol-6-yl) thiophen-3-yl)benzene- sulfonamide | 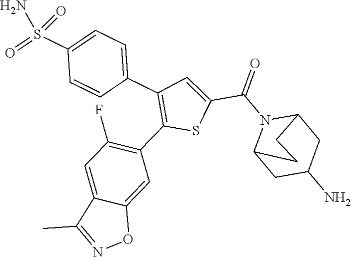 [SMILES: Cc1noc2cc(-c3sc(C(=O)N4C5CCC4CC(N)C5)cc3-c3ccc(S(N)(=O)=O)cc3)c(F)cc12] | ¹H NMR (400 MHz, DMSO-d₆) δ ppm 7.95 (d, J = 5.4 Hz, 1H), 7.80 (d, J = 8.8 Hz, 1H), 7.78-7.67 (m, 3H), 7.53-7.45 (m, 4H), 4.68 (s, 2H), 3.14 (tt, J = 11.0, 5.6 Hz, 1H), 2.55 (s, 3H), 2.04- 1.40 (m, 8H). | 541.1 |
| 213 | (3-amino-8-azabicyclo [3.2.1]octan-8-yl) (5-(5-fluoro-3-methylbenzo [d]isoxazol-6-yl)-4-(4- (methyldimethylsulfoxide- 16-sulfonyl)phenyl)thio- phen-2-yl)methanone | 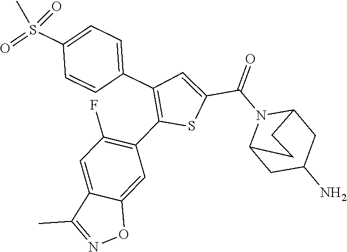 [SMILES: Cc1noc2cc(-c3sc(C(=O)N4C5CCC4CC(N)C5)cc3-c3ccc(S(C)(=O)=O)cc3)c(F)cc12] | ¹H NMR (400 MHz, DMSO-d₆) δ ppm 7.98 (d, J = 5.6 Hz, 1H), 7.83 (dd, J = 13.8, 8.6 Hz, 3H), 7.75 (s, 1H), 7.64-7.45 (m, 2H), 4.69 (s, 2H), 3.22 (s, 4H), 2.55 (s, 3H), 1.82 (d, J = 47.8 Hz, 8H). | 540.1 |
| 214 | 4-(5-(3-amino-8-azabicyclo [3.2.1]octan-8-carbonyl)- 2-(2-carbonyl-2,3- dihydrobenzo[d]oxazol-5- yl)thiophen-3-yl)-2-fluoro benzonitrile hydrochloride | 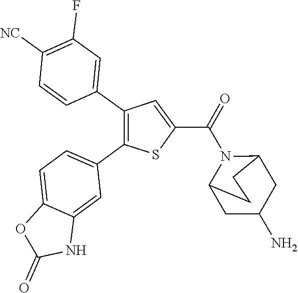 [SMILES: N#Cc1ccc(-c2cc(C(=O)N3C4CCC3CC(N)C4)sc2-c2ccc3oc(=O)[nH]c3c2)cc1F] | ¹H NMR (400 MHz, DMSO-d₆) δ ppm 11.75 (d, J = 7.6 Hz, 1H), 8.11-7.83 (m, 4H), 7.71 (s, 1H), 7.62 (dd, J = 10.7, 1.6 Hz, 1H), 7.33 (s, 1H), 7.23 (dd, J = 8.0, 1.6 Hz, 1H), 7.03 (dd, J = 8.4, 1.8 Hz, 1H), 6.97 (d, J = 1.8 Hz, 1H), 4.77 (s, 2H), 3.63 (d, J = 5.2 Hz, 1H), 1.96 (d, J = 40.4 Hz, 4H), 1.82- 1.64 (m, 4H). | 489.1 |
| 215 | 4-(5-(3-amino-8-azabicyclo [3.2.1]octan-8-carbonyl)- 2-(1-(cyanomethyl)-5- fluoro-1H-indazol-6- yl)thiophen-3-yl)-2-fluoro- benzonitrile | 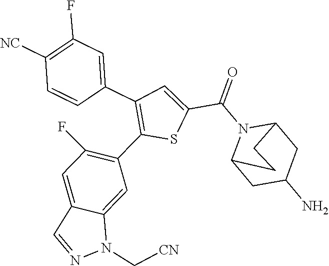 [SMILES: N#CCn1ncc2cc(F)c(-c3sc(C(=O)N4C5CCC4CC(N)C5)cc3-c3ccc(C#N)c(F)c3)cc21] | ¹H NMR (400 MHz, DMSO-d₆) δ ppm 8.28 (s, 1H), 8.10 (d, J = 5.8 Hz, 1H), 7.88-7.75 (m, 2H), 7.70 (d, J = 9.8 Hz, 1H), 7.61 (d, J = 10.8 Hz, 1H), 7.20 (d, J = 8.2 Hz, 1H), 5.83 (s, 2H), 4.70 (s, 2H), 3.13 (dq, J = 11.2, 5.6 Hz, 1H), 2.14-1.63 (m, 8H). | 529.2 |
| 216 | 4-(5-(3-amino-8-azabicyclo [3.2.1]octan-8-carbonyl)- 2-(5-fluoro-1H-benzo [d][1,2,3]triazol-6-yl)thio- phen-3-yl)-2-fluorobenzo- nitrile hydrochloride | 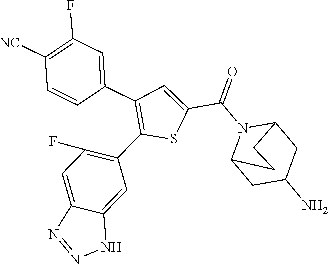 [SMILES: N#Cc1ccc(-c2cc(C(=O)N3C4CCC3CC(N)C4)sc2-c2cc3[nH]nnc3cc2F)cc1F] | 1H NMR (400 MHz, Methanol-d₄) δ 8.09 (s, 1H), 7.78 (s, 1H), 7.63 (t, J = 7.8 Hz, 2H), 7.35 (d, J = 9.8 Hz, 1H), 7.22 (d, J = 8.0 Hz, 1H), 3.80 (dd, J = 12.2, 6.6 Hz, 2H), 3.52-3.44 (m, 1H), 2.04 (s, 2H), 1.93 (dd, J = 12.4, 5.8 Hz, 4H), 1.60 (s, 2H). | 491.1 |
| 217 | 4-(5-(3-amino-8-azabicyclo [3.2.1]octan-8-carbonyl)- 2-(5-fluoro-1-(2- hydroxy-2-methylpropyl)- 1H-indazol-6-yl)thiophen- 3-yl)-2-fluorobenzonitrile hydrochloride | 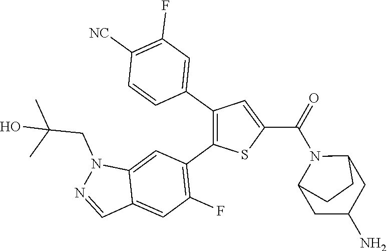 [SMILES: CC(C)(O)Cn1ncc2cc(F)c(-c3sc(C(=O)N4C5CCC4CC(N)C5)cc3-c3ccc(C#N)c(F)c3)cc21] | ¹H NMR (400 MHz, DMSO-d₆) δ ppm 8.02 (d, J = 15.6 Hz, 4H), 7.86 (d, J = 5.8 Hz, 1H), 7.81-7.67 (m, 2H), 7.62-7.47 (m, 2H), 7.15 (dd, J = 8.0, 1.6 Hz, 1H), 4.72 (s, 3H), 4.27 (s, 2H), 3.62-3.51 (m, 1H), 1.98 (s, 4H), 1.72 (t, J = 12.8 Hz, 4H), 1.01 (s, 6H). | 562.2 |
| 218 | 4-(5-(3-amino-8-azabicyclo [3.2.1]octan-8-carbonyl)- 2-(3-hydroxy-4-methoxy- phenyl)thiophen-3-yl)- 2-fluorobenzonitrile | 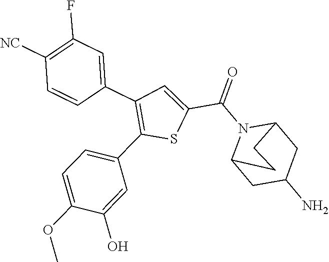 [SMILES: COc1ccc(-c2sc(C(=O)N3C4CCC3CC(N)C4)cc2-c2ccc(C#N)c(F)c2)cc1O] | ¹H NMR (400 MHz, DMSO-d₆) δ ppm 7.99-7.77 (m, 1H), 7.65-7.48 (m, 2H), 7.28 (dd, J = 8.0, 1.6 Hz, 1H), 6.94 (d, J = 8.4 Hz, 1H), 6.81-6.54 (m, 2H), 4.66 (s, 2H), 3.79 (s, 3H), 3.12 (tt, J = 11.0, 5.4 Hz, 1H), 2.18- 1.48 (m, 8H), 1.40 (s, 2H). | 478.2 |

### Example 219

### Preparation of 4-(5-(3-amino-8-azabicyclo[3.2.1]octane-8-carbonyl)-2-(5-fluoro-3-methylbenzo[d]isoxazol-6-yl) furan-3-yl)-2-fluorobenzonitrile hydrochloride

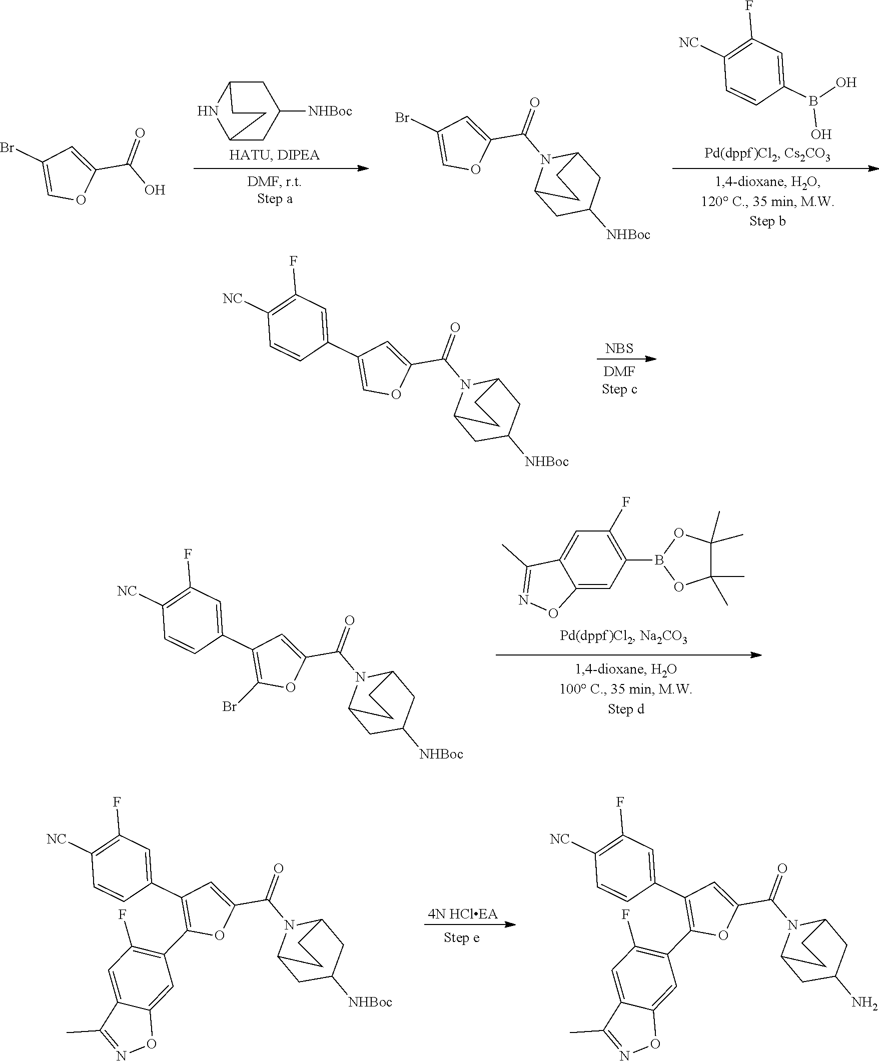

### Step a): Preparation of tert-butyl(8-(4-bromofuran-2-carbonyl)-8-azabicyclo[3.2.1]octane-3-yl)carbamate

4-bromofuran-2-carboxylic acid (500 mg, 2.63 mmol) and tert-butyl (8-azabicyclo[3.2.1]octane-3-yl)carbamate (650 mg, 2.879 mmol) are dissolved in N,N-dimethylformamide (20 mL), added with HATU (1.29 g, 3.40 mmol) and DIPEA (1.15 mL, 7.08 mmol), and stirred for 30 min at room temperature; when TLC and LC-MS show that the reaction is completed, the reaction solution is quenched with water, extracted with ethyl acetate (80 mL×3), washed with 80 mL of saline, dried with anhydrous sodium sulfate, filtered and concentrated to obtain a crude product; and the residue is purified by silica gel chromatography (eluent: petroleum ether/ethyl acetate=10/3) to obtain tert-butyl(8-(4-bromofuran-2-carbonyl)-8-azabicyclo[3.2.1]octane-3-yl)carbamate with a yield of 96.4%.

ESI-MS(m/z)=399.1[M+H]⁺.

### Step b): Preparation of tert-butyl(8-(4-(4-cyano-3-fluorophenyl)furan-2-carbonyl)-8-azabicyclo[3.2.1]octane-3-yl)carba mate

Tert-butyl(8-(4-(4-cyano-3-fluorophenyl)furan-2-carbonyl)-8-azabicyclo[3.2.1]octane-3-yl)carba mate (1 g, 2.5 mmol), (4-cyano-3-fluorophenyl)boronic acid (0.581 g, 3.5 mmol), Cs₂CO₃ (2.86 g, 8.79 mmol), and Pd(dppf)Cl₂ (186 mg, 0.25 mmol) are dissolved in 14 mL of dioxane and added with 0.4 mL of water; and this mixed reaction solution is purged by nitrogen blowing, warmed to 120° C., and reacted for 45 min while stirring. When the reaction is completed, the filtrate is concentrated under a reduced pressure; and the residue is purified by silica gel chromatography (eluent: petroleum ether/ethyl acetate=2/1) to obtain tert-butyl(8-(4-(4-cyano-3-fluorophenyl)furan-2-carbonyl)-8-azabicyclo[3.2.1]octan-3-yl)carbam ate with a yield of 83.2%.

ESI-MS(m/z)=440.2[M+H]⁺.

### Step c): Preparation of tert-butyl(8-(5-4-(4-cyano-3-fluorophenyl)furan-2-carbonyl)-8-azabicyclo[3.2.1]octane-3-yl)carbamate

Tert-butyl(8-(4-(4-cyano-3-fluorophenyl)furan-2-carbonyl)-8-azabicyclo[3.2.1]octane-3-yl)carba mate (300 mg, 0.683 mmol) is dissolved in N,N-dimethylformamide (10 mL), added with NBS (136 mg, 0.751 mmol) at 0° C. and stirred at room temperature; and LS-MS shows that the reaction is completed. The reaction solution is quenched with water (50 mL) and extracted with ethyl acetate (50 mL×2); organic layers are combined and then washed with saturated saline (60 mL×2), dried with anhydrous sodium sulfate, filtered, and concentrated to obtain a crude product; and the residue is purified by silica gel chromatography (eluent: petroleum ether/ethyl acetate=2/1) to obtain tert-butyl(8-(5-4-(4-cyano-3-fluorophenyl)furan-2-carbonyl)-8-azabicyclo[3.2.1]octane-3-yl)carbamate with a yield of 64.5%.

ESI-MS m/z)=518.1 [M+H]⁺.

### Step d): Preparation of tert-butyl(8-(4-(4-cyano-3-fluorophenyl)-5-(5-fluoro-3-methylbenzo[d]isoxazol-6-yl)furan-2-car bonyl)-8-azabicyclo[3.2.1]octane-3-yl)carbamate

Tert-butyl(8-(5-bromo-4-(4-cyano-3-fluorophenyl)furan-2-carbonyl)-8-azabicyclo[3.2.1]octan-3-yl)carbamate (70 mg, 0.135 mmol), 5-fluoro-3-methyl-6-(4,4,5,5-tetramethyl-1,3,2-dioxaborolane-2-yl)benzo[d]isoxazole (75 mg, 0.27 mmol), Na₂CO₃ (43 mg, 0.406 mmol), and Pd(dppf)Cl₂ (10 mg, 0.0137 mmol) are dissolved in 4 mL of dioxane and added with 0.4 mL of water; and this mixed reaction solution is purged by nitrogen blowing, warmed to 100° C., and reacted for 0.5 h while stirring. When the reaction is completed, the reaction solution is concentrated under a reduced pressure; and the residue is purified by silica gel chromatography (eluent: petroleum ether/ethyl acetate=10/7) to obtain tert-butyl(8-(4-(4-cyano-3-fluorophenyl)-5-(5-fluoro-3-methylbenzo[d]isoxazol-6-yl)furan-2-car bonyl)-8-azabicyclo[3.2.1]octane-3-yl)carbamate with a yield of 67.2%.

ESI-MS(m/z)=589.2[M+H]⁺

### Step e): Preparation of 4-(5-(3-amino-8-azabicyclo[3.2.1]octane-8-carbonyl)-2-(5-fluoro-3-methylbenzo[d]isoxazol-6-yl) furan-3-yl)-2-fluorobenzonitrile hydrochloride

Tert-butyl(8-(4-(4-cyano-3-fluorophenyl)-5-(5-fluoro-3-methylbenzo[d]isoxazol-6-yl) furan-2-carbonyl)-8-azabicyclo[3.2.1]octane-3-yl)carbamate (52 mg, 0.11 mmol) is added to 10 mL of 4M hydrochloric acid (ethyl acetate solution), and reacted for 2 h at room temperature, a small amount of solids are formed in the reaction system, and LC-MS shows that the reaction is completed. After the solvent is dried by spinning, the reaction solution is added with 4 mL of acetonitrile and 5 mL of pure water and freeze-dried to obtain 4-(5-(3-amino-8-azabicyclo[3.2.1]octane-8-carbonyl)-2-(5-fluoro-3-methylbenzo[d]isoxazol-6-yl) furan-3-yl)-2-fluorobenzonitrile hydrochloride with a yield of 92.5%.

¹H NMR (400 MHz, DMSO-d₆) δ ppm 8.07 (d, J=5.1 Hz, 1H), 8.03-7.80 (m, 5H), 7.76-7.62 (m, 2H), 7.37 (dd, J=17.8, 8.2 Hz, 1H), 5.02 (s, 1H), 4.74 (s, 1H), 3.63 (s, 1H), 2.59 (s, 3H), 2.14-1.90 (m, 4H), 1.76 (s, 4H).

ESI-MS m/z=489.2 [M+H]⁺.

### Example 220

### Preparation of 4-(2-(3-amino-8-azabicyclo[3.2.1]octane-8-carbonyl)-5-(5-fluoro-3-methylbenzo[d]isoxazol-6-yl) thiazol-4-yl)-2-fluorobenzonitrile hydrochloride

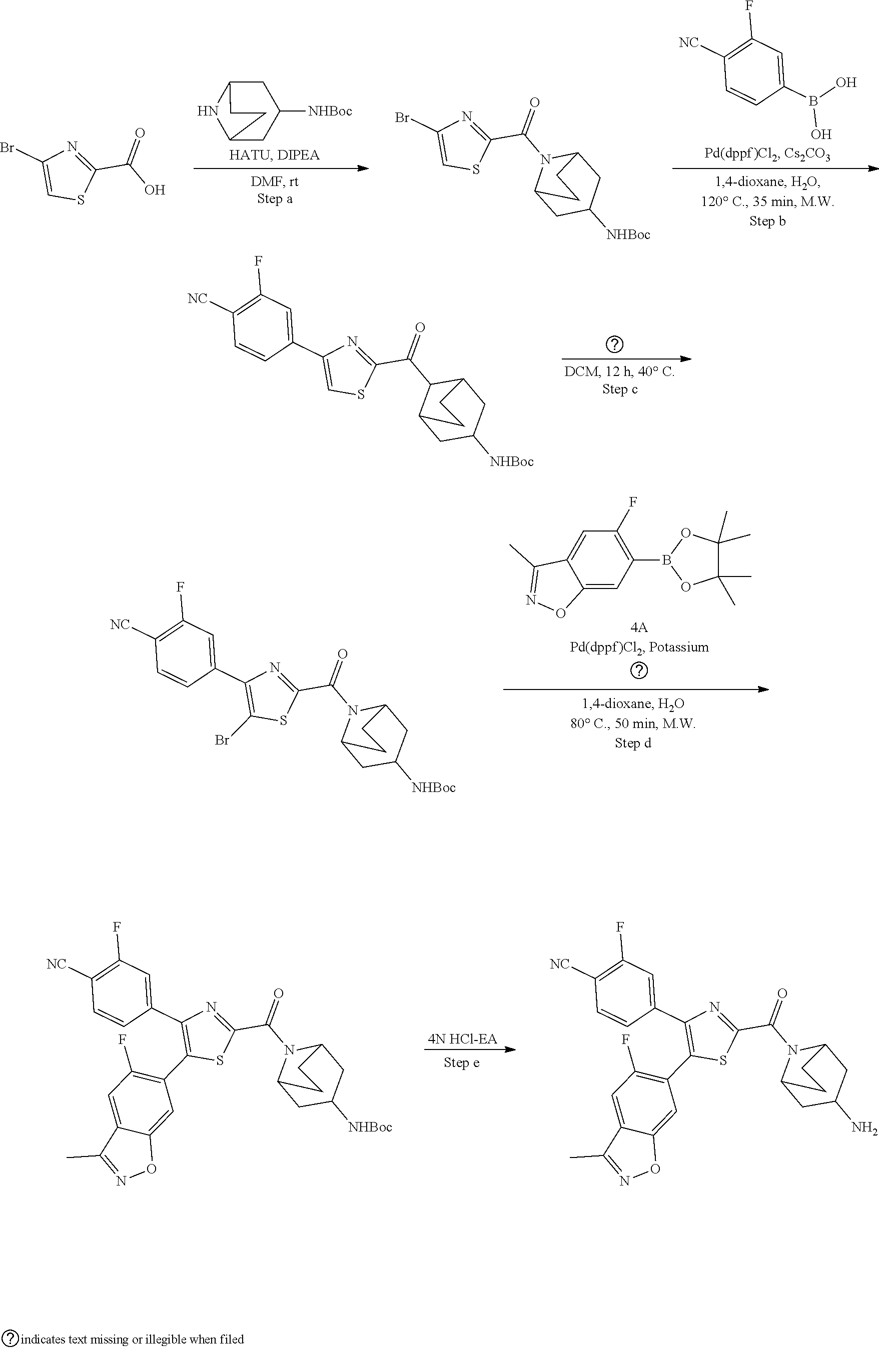

### Step a): Preparation of tert-butyl(8-(4-bromothiazol-2-carbonyl)-8-azabicyclo[3.2.1]octane-3-yl)carbamate

4-bromothiazole-2-carboxylic acid (300 mg, 1.44 mmol) and tert-butyl (8-azabicyclo[3.2.1]octane-3-yl)carbamate (360 mg, 1.58 mmol) are dissolved in N,N-dimethylformamide (8 mL), added with HATU (173 mg, 1.87 mmol) and DIPEA (558 mg, 4.3 mmol), and stirred for 30 min at room temperature; when TLC and LC-MS show that the reaction is completed, the reaction solution is quenched with water, extracted with ethyl acetate (40 mL×3), washed with 50 mL of saline, dried with anhydrous sodium sulfate, filtered and concentrated to obtain a crude product; and the residue is purified by silica gel chromatography (eluent: petroleum ether/ethyl acetate=10/7) to obtain tert-butyl(8-(4-bromothiazol-2-carbonyl)-8-azabicyclo[3.2.1]octane-3-yl)carbamate with a yield of 96.4%.

ESI-MS(m/z)=416.1[M+H]⁺.

### Step b): Preparation of tert-butyl(8-(4-(4-cyano-3-fluorophenyl)thiazol-2-carbonyl)bicyclo[3.2.1]octane-3-yl) carbamate

Tert-butyl(8-(4-bromothiazol-2-carbonyl)-8-azabicyclo[3.2.1]octan-3-yl)carbamate (0.6 g, 1.4 mmol), (4-cyano-3-fluorophenyl)boronic acid (0.334 g, 2.02 mmol), Cs₂CO₃ (1.6 g, 5.06 mmol), and Pd(dppf)Cl₂ (106 mg, 0.144 mmol) are dissolved in 14 mL of dioxane and added with 0.4 mL of water; and this mixed reaction solution is purged by nitrogen blowing, warmed to 120° C., and reacted for 0.5 h while stirring. When the reaction is completed, the reaction solution is concentrated under a reduced pressure; and the residue is purified by silica gel chromatography (eluent: dichloromethane/ethyl acetate=10/3) to obtain tert-butyl(8-(4-(4-cyano-3-fluorophenyl)thiazol-2-carbonyl)bicyclo[3.2.1]octane-3-yl) carbamate with a yield of 83.5%.

ESI-MS m/z)=456.2 [M+H]⁺.

### Step c): Preparation of tert-butyl(8-(5-bromo-4-(4-cyano-3-fluorophenyl)thiazol-2-carbonyl)-8-azabicyclo[3.2.1]octane-3-yl)carbamate

Tert-butyl(8-(4-(4-cyano-3-fluorophenyl)thiazol-2-carbonyl)bicyclo[3.2.1]octane-3-yl) carbamate (262 mg, 0.58 mmol) is dissolved in dichloromethane (10 mL), added with dibromohydantoin (166 mg, 0.575 mmol) and trifluoroacetic acid (65 mg, 0.575 mmol) and stirred at room temperature; when LS-MS shows that the reaction is completed. The reaction solution is quenched with saturated sodium bicarbonate, extracted with ethyl acetate (40 mL×3), then washed with 50 mL of saline, dried, filtered and concentrated under a reduced pressure to obtain residue; and the residue is purified by silica gel chromatography (eluent: dichloromethane/ethyl acetate=5/3) to obtain tert-butyl(8-(5-bromo-4-(4-cyano-3-fluorophenyl)thiazol-2-carbonyl)-8-azabicyclo[3.2.1]octane-3-yl)carbamate with a yield of 64.5%.

ESI-MS(m/z)=535.1[M+H]⁺.

### Step d): Preparation of tert-butyl(8-(4-(4-cyano-3-fluorophenyl)-5-(5-fluoro-3-methylbenzo[d]isoxazol-6-yl)thiazol-2-ca rbonyl)-8-azabicyclo[3.2.1]octan-3-yl)carbamate

Tert-butyl(8-(5-bromo-4-(4-cyano-3-fluorophenyl)thiazol-2-carbonyl)-8-azabicyclo[3.2.1]octane-3-yl)carbamate (45 mg, 0.084 mmol), 5-fluoro-3-methyl-6-(4,4,5,5-tetramethyl-1,3,2-dioxaborolane-2-yl)benzo[d]isoxazole (47 mg, 0.168 mmol), potassium trimethylsilanolate (22 mg, 0.17 mmol) and Pd(dppf)Cl₂ (6 mg, 0.0084 mmol) are dissolved in 4 mL of dioxane and added with 0.4 mL of water; and this mixed reaction solution is purged by nitrogen blowing, warmed to 80° C., and reacted for 1 while stirring. When the reaction is completed, the reaction solution is concentrated under a reduced pressure; and the residue is purified by silica gel chromatography (eluent: dichloromethane/ethyl acetate=5/3) to obtain tert-butyl(8-(4-(4-cyano-3-fluorophenyl)-5-(5-fluoro-3-methylbenzo[d]isoxazol-6-yl)thiazol-2-ca rbonyl)-8-azabicyclo[3.2.1]octane-3-yl)carbamate with a yield of 39.5%.

ESI-MS(m/z)=606.2[M+H]⁺

### Step e): Preparation of 4-(2-(3-amino-8-azabicyclo[3.2.1]octane-8-carbonyl)-5-(5-fluoro-3-methylbenzo[d]isoxazol-6-yl) thiazol-4-yl)-2-fluorobenzonitrile hydrochloride

Tert-butyl(8-(4-(4-cyano-3-fluorophenyl)-5-(5-fluoro-3-methylbenzo[d]isoxazol-6-yl)thiazol-2-c arbonyl)-8-azabicyclo[3.2.1]octane-3-yl)carbamate (52 mg, 0.11 mmol) is added to 10 mL of 4M hydrochloric acid (ethyl acetate solution), and reacted for 1 h at room temperature, a small amount of solids are formed in the reaction system. When LC-MS shows that the reaction is completed, the solvent is dried by spinning, the reaction solution is delivered to Prep-HPLC preparation (separation method 1) to obtain 4-(2-(3-amino-8-azabicyclo[3.2.1]octane-8-carbonyl)-5-(5-fluoro-3-methylbenzo[d]isoxazol-6-yl) thiazol-4-yl)-2-fluorobenzonitrile hydrochloride with a yield of 65.3%.

¹H NMR (400 MHz, DMSO-d₆) δ ppm 8.11 (s, 3H), 8.03 (s, 1H), 7.97-7.77 (m, 2H), 7.61 (d, J=10.6 Hz, 1H), 7.36 (d, J=8.2 Hz, 1H), 5.69 (d, J=7.2 Hz, 1H), 4.75 (d, J=6.8 Hz, 1H), 3.82-3.68 (m, 1H), 2.50 (q, J=1.8 Hz, 3H), 2.19-1.95 (m, 4H), 1.81 (dt, J=55.4, 12.2 Hz, 4H).

ESI-MS m/z=506.2 [M+H]⁺.

### Example 221

### 4-(2-(3-amino-8-azabicyclo[3.2.1]octan-8-carbonyl)-5-(1-(cyanomethyl)-5-fluoro-1H-indazol-6-yl)thiazol-4-yl)-2-fluorobenzonitrile hydrochloride is prepared according to the synthesis method (separation method 1) of Example 220, and the structure and representation data are as follows

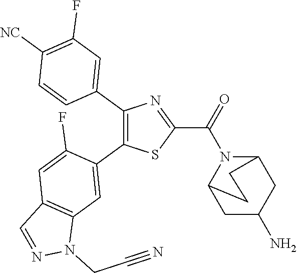 [SMILES: N#CCn1ncc2cc(F)c(-c3sc(C(=O)N4C5CCC4CC(N)C5)nc3-c3ccc(C#N)c(F)c3)cc21]

¹H NMR (400 MHz, Methanol-d₄) δ ppm 8.22 (d, J=1.0 Hz, 1H), 7.93 (d, J=5.6 Hz, 1H), 7.72-7.60 (m, 2H), 7.53 (dd, J=10.6, 1.6 Hz, 1H), 7.38 (dd, J=8.2, 1.6 Hz, 1H), 5.98-5.83 (m, 1H), 5.61 (s, 2H), 4.95 (dd, J=7.4, 3.4 Hz, 1H), 3.84 (tt, J=11.6, 5.8 Hz, 1H), 2.33-2.10 (m, 4H), 2.10-1.84 (m, 4H).

ESI-MS m/z=530.2 [M+H]⁺.

### Example 222

### Preparation of 4-(2-(3-amino-8-azabicyclo[3.2.1]octane-8-carbonyl)-5-(5-fluoro-3-methylbenzo[d]isoxazol-6-yl) isoxazol-4-yl)-2-fluorobenzonitrile hydrochloride

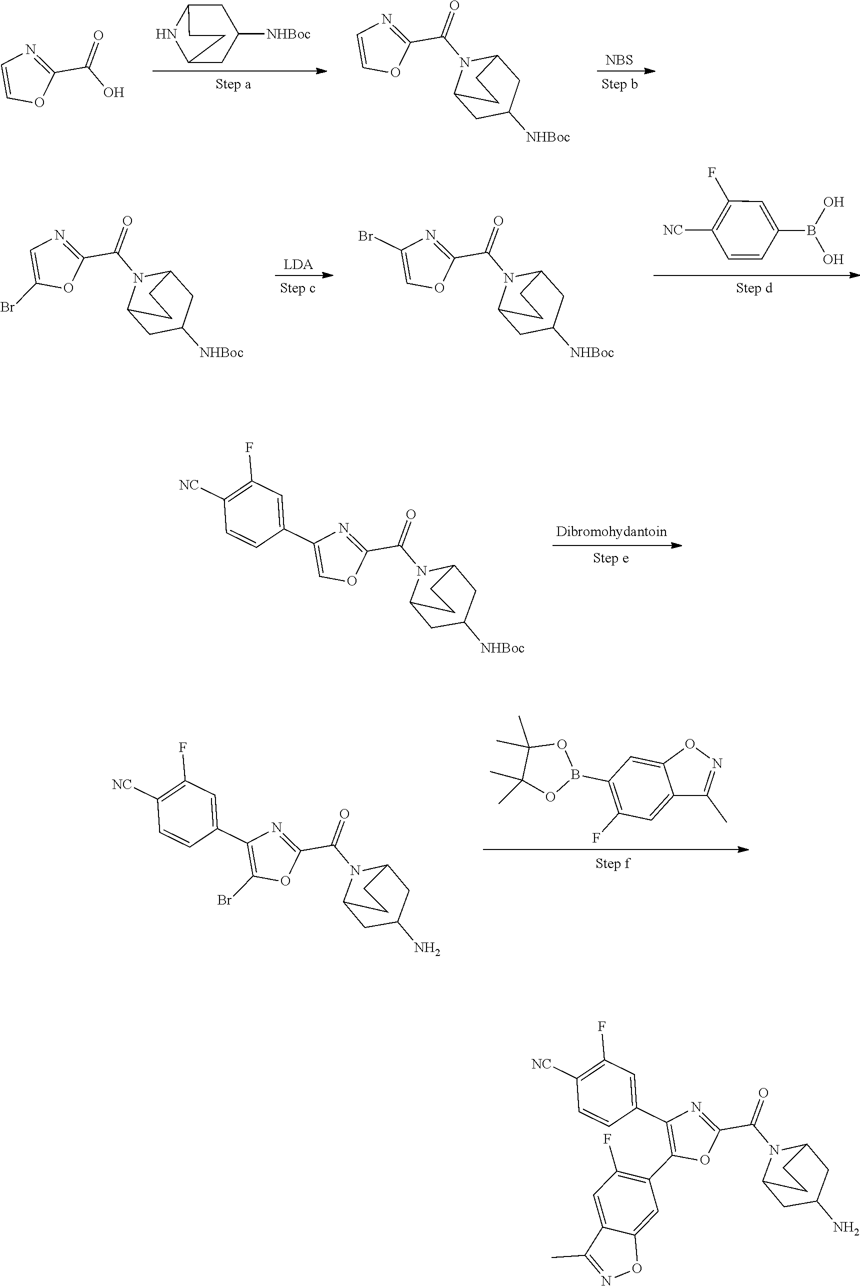

### Step a): Preparation of tert-butyl 8-(oxazol-2-carbonyl)-8-azabicyclo[3.2.1]octane-3-yl)carbamate

Oxazole-2-carboxylic acid (1.0 g, 8.844 mmol) tert-butyl (8-azabicyclo[3.2.1]octane-3-yl)carbamate (2.2 g, 9.728 mmol), and HATU (4.37 g, 11.497 mmol) are dissolved in DMF (50 mL) at 0° C., and added with DIEA (3.42 g, 26.532 mmol) while stirring at 0° C., and the reaction is maintained for 1 h at room temperature. When the reaction is completed, the reaction solution is quenched with water (300 mL), and extracted with ethyl acetate (50 mL×2); organic phase is combined, washed with saturated saline (100 mL×2), dried with anhydrous sodium sulfate, and filtered to obtain filtrate; the filtrate is concentrated under a reduced pressure; and the residue is purified by silica gel chromatography (eluent: ethyl acetate/petroleum ether=1/1) to obtain tert-butyl 8-(oxazol-2-carbonyl)-8-azabicyclo[3.2.1]octane-3-yl)carbamate with a yield of 91.6%.

¹H NMR (400 MHz, DMSO-d₆) δppm 8.31 (s, 1H), 7.47 (s, 1H), 6.74 (d, J=9.4 Hz, 1H), 5.21-5.20 (m, 1H), 4.65-4.62 (m, 1H), 3.92-3.88 (m, 1H), 2.07-1.93 (m, 1H), 1.91-1.74 (m, 5H), 1.65-1.48 (m, 2H), 1.36 (s, 9H).

ESI-MS(m/z)=322.1 [M+H]⁺.

### Step b): Preparation of tert-butyl (8-(5-bromooxazol-2-carbonyl)-8-azabicyclo[3.2.1]octane-3-yl)carbamate

Tert-butyl (8-(oxazole-2-carbonyl)-8-azabicyclo[3.2.1]octane-3-yl)carbamate (700 mg, 2.180 mmol) and tetrahydrofuran (10 mL) are added to a reaction flask, cooled to −78° C., added dropwise with n-butyllithium (2.7 mL, 1.6 M, 4.362 mmol), and stirred at −78° C. for 30 min. NBS (815 mg, 4.579 mmol) is dissolved in THE (2 mL), then added dropwise into a reaction flask and maintained to react at −78° C. for 4 h. When the reaction is completed, the reaction solution is quenched with saturated sodium sulfite solution (20 mL), and extracted with ethyl acetate (10 mL×2); organic phase is combined, washed with saturated saline (10 mL×2), dried with anhydrous sodium sulfate, and filtered to obtain filtrate; the filtrate is concentrated under a reduced pressure; and the residue is purified by silica gel chromatography (eluent: ethyl acetate/petroleum ether=1/2) to obtain tert-butyl (8-(5-bromooxazol-2-carbonyl)-8-azabicyclo[3.2.1]octane-3-yl)carbamate with a yield of 14.3%.

¹H NMR (400 MHz, DMSO-d₆) δppm 7.58 (s, 1H), 6.73 (d, J=8.2 Hz, 1H), 5.13-5.12 (m, 1H), 4.63-4.62 (m, 1H), 3.96-3.76 (m, 1H), 1.99-1.98 (m, 1H), 1.90-1.73 (m, 5H), 1.65-1.46 (m, 2H), 1.36 (s, 9H).

ESI-MS(m/z)=400.2 [M+H]⁺.

### Step c): Preparation of tert-butyl (8-(4-bromooxazol-2-carbonyl)-8-azabicyclo[3.2.1]octane-3-yl)carbamate

Tert-butyl (8-(5-bromooxazol-2-carbonyl)-8-azabicyclo[3.2.1]octane-3-yl)carbamate (110 mg, 0.276 mmol) and tetrahydrofuran (4 mL) are added to a reaction flask, cooled to −78° C., added dropwise with lithium diisopropylamide (0.3 mL, 2.0 M, 0.407 mmol), and stirred at −78° C. for 4 h. When the reaction is completed, the reaction solution is quenched with a saturated ammonium chloride solution (10 mL), and extracted with ethyl acetate (10 mL×2); organic phase is combined, washed with saturated saline (10 mL×2), dried with anhydrous sodium sulfate, and filtered to obtain filtrate; the filtrate is concentrated under a reduced pressure; and the residue is purified by silica gel chromatography (eluent: ethyl acetate/petroleum ether=1/3) to obtain tert-butyl (8-(4-bromooxazol-2-carbonyl)-8-azabicyclo[3.2.1]octane-3-yl)carbamate with a yield of 36.3%.

¹H NMR (400 MHz, DMSO-d6) δ 8.57 (s, 1H), 6.72 (d, J=8.0 Hz, 1H), 5.07-5.06 (m, 1H), 4.64-4.62 (m, 1H), 3.96-3.81 (m, 1H), 2.07-1.95 (m, 1H), 1.92-1.65 (m, 5H), 1.64-1.44 (m, 2H), 1.36 (s, 9H).

ESI-MS(m/z)=400.2 [M+H]⁺.

### Step d): Preparation of tert-butyl (8-(4-(4-cyano-3-fluorophenyl)oxazole-2-carbonyl)-8-azadicyclo[3.2.1]octyl-3-yl)carbamate

Tert-butyl (8-(4-bromooxazol-2-carbonyl)-8-azabicyclo[3.2.1]octane-3-yl)carbamate (35 mg, 0.0877 mmol), (4-cyano-3-fluorophenyl)boronic acid (29 mg, 0.175 mmol), Cs₂CO₃ (85 mg, 0.263 mmol), Pd(dppf)Cl₂ (6.4 mg, 0.00877 mmol), 1,4-dioxane (2 mL) and water (1 mL) are sequentially added in a reaction flask, subjected to nitrogen displacement for three times, warmed to 110° C. and reacted for 2 h while stirring. When the reaction is completed, the reaction solution is quenched with water (10 mL), and extracted with ethyl acetate (10 mL×2); organic phase is combined, washed with saturated saline (10 mL×2), dried with anhydrous sodium sulfate, and filtered to obtain filtrate; the filtrate is concentrated under a reduced pressure; and the residue is purified by silica gel chromatography (eluent: petroleum ether/ethyl acetate=1/1) to obtain the compound tert-butyl (8-(4-(4-cyano-3-fluorophenyl)oxazole-2-carbonyl)-8-azabicyclo[3.2.1]octane-3-yl)carbamate with a yield of 57.1%.

¹H NMR (400 MHz, DMSO-d₆) δppm 9.05 (s, 1H), 8.05 (t, J=9.0 Hz, 1H), 7.96 (d, J=10.4 Hz, 1H), 7.87 (d, J=8.0 Hz, 1H), 6.73 (d, J=8.4 Hz, 1H), 5.27-5.25 (m, 1H), 4.69-4.64 (m, 1H), 3.99-3.86 (m, 1H), 2.10-1.98 (m, 1H), 1.97-1.73 (m, 5H), 1.68 (t, J=12.8 Hz, 1H), 1.55 (t, J=12.0 Hz, 1H), 1.36 (s, 9H).

ESI-MS(m/z)=441.4 [M+H]⁺.

### Step e): Preparation of 4-(2-(3-amino-8-azabicyclo[3.2.1]octane-8-carbonyl)-5-bromooxazol-4-yl)-2-fluorobenzonitrile

### Tert-butyl

(8-(4-(4-cyano-3-fluorophenyl)oxazole-2-carbonyl)-8-azabicyclo[3.2.1]octyl-3-yl)carbamate (50 mg, 1.144 mmol), dichloromethane (2 mL) and trifluoroacetic acid (0.5 mL) are added to a reaction flask; and the reaction solution is then added with 1,3-dibromo-5,5-dimethylhydantoin (38 mg, 0.136 mmol), and reacted at room temperature for 16 h. When the reaction is completed, the reaction solution is quenched with a saturated sodium bicarbonate aqueous solution (10 mL), and extracted with dichloromethane (10 mL×3); organic phase is combined, washed with saturated saline (10 mL×2), dried with anhydrous sodium sulfate, and filtered to obtain filtrate; the filtrate is concentrated under a reduced pressure; and the residue is purified by a C18 silica gel column (eluent: acetonitrile/water=1.5/1) to obtain 4-(2-(3-amino-8-azabicyclo[3.2.1]octane-8-carbonyl)-5-bromooxazol-4-yl)-2-fluorobenzonitrile with a yield of 79.5%.

ESI-MS m/z)=419.0 [M+H]⁺.

### Step f): Preparation of 4-(2-(3-amino-8-azabicyclo[3.2.1]octane-8-carbonyl)-5-(5-fluoro-3-methylbenzo[d]isoxazol-6-yl) oxazol-4-yl)-2-fluorobenzonitrile hydrochloride

(4-(2-(3-amino-8-azabicyclo[3.2.1]octane-8-carbonyl)-5-bromooxazol-4-yl)-2-fluorobenzonitrile (38 mg, 0.090 mmol), 5-fluoro-3-methyl-6-(4,4,5,5-tetramethyl-1,3,2-dioxaborolane-2-yl)benzo[d]isoxazole (62 mg, 0.227 mmol), Pd(dppf)Cl₂ (8.3 mg, 0.0113 mmol), potassium trimethylsilanolate (29 mg, 0.227 mmol), and 1,4-dioxane (2 mL) are sequentially dissolved in a reaction flask, subjected to nitrogen displacement for three times, warmed to 80° C. and reacted for 2 h while stirring. When the reaction is completed, the reaction solution is quenched with water (10 mL), and extracted with ethyl acetate (5 mL×3); organic phase is combined, washed with saturated saline (10 mL×1), dried with anhydrous sodium sulfate, and filtered to obtain filtrate; the filtrate is concentrated under a reduced pressure; and the residue is purified by Prep-HPLC (separation method 4) to obtain the compound 4-(2-(3-amino-8-azabicyclo[3.2.1]octane-8-carbonyl)-5-(5-fluoro-3-methylbenzo[d]isoxazol-6-yl) oxazol-4-yl)-2-fluorobenzonitrile hydrochloride with a yield of 34.1%.

¹H NMR (400 MHz, Methanol-d₄) δppm 8.03 (d, J=4.8 Hz, 1H), 7.84-7.75 (m, 2H), 7.64 (d, J=10.4 Hz, 1H), 7.53 (d, J=8.0 Hz, 1H), 5.67-5.66 (m, 1H), 5.00-4.98 (m, 1H), 3.88-3.82 (m, 1H), 2.64 (s, 3H), 2.35-2.23 (m, 2H), 2.21-1.83 (m, 6H).

ESI-MS(m/z)=490.2 [M+H]⁺.

### Example 223

### Preparation of 4-(2-(4-aminopiperidin-1-yl)-5-(3-hydroxy-4-methoxyphenyl)thiazol-4-yl)-2-fluorobenzonitrile

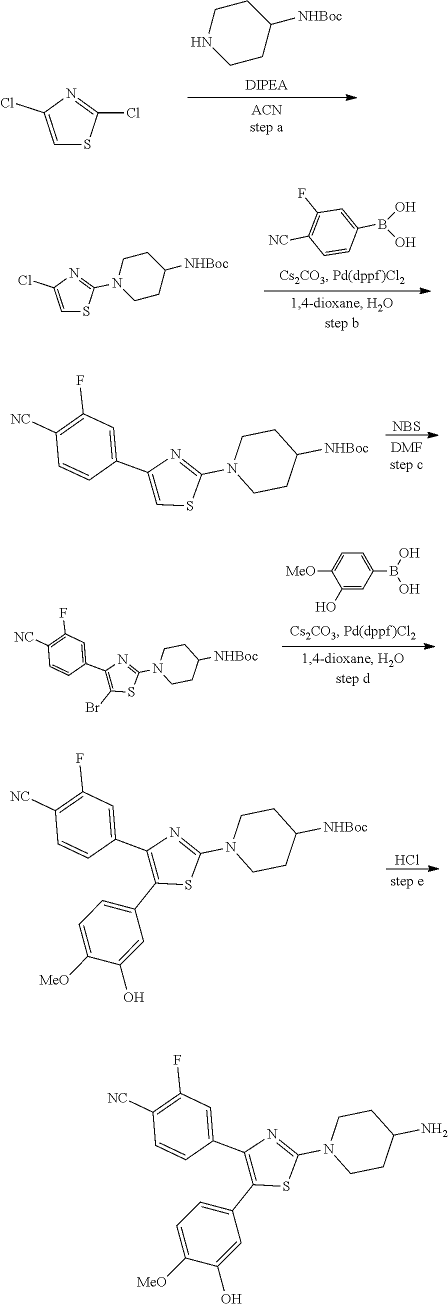

### Step a): Preparation of tert-butyl(1-(4-chlorothiazol-2-yl)piperidin-4-yl)carbamate

2,4-dichlorothiazole (800 mg, 5.2 mmol) and DIPEA (1.3 g, 10.4 mmol) are dissolved in acetonitrile (30 mL), added with tert-butylpiperidin-4-yl-carbamate (1.03 g, 5.2 mmol) in an ice bath, reacted overnight at room temperature, and dried by spinning; and the residue is then purified by silica gel chromatography (eluent: petroleum ether/ethyl acetate=5/3) to obtain tert-butyl(1-(4-chlorothiazol-2-yl)piperidin-4-yl)carbamate with a yield of 50.2%.

ESI-MS m/z=318.1 [M+H]⁺.

### Step b): Preparation of tert-butyl(1-(4-(4-cyano-3-fluorophenyl)thiazol-2-yl)piperidin-4-yl)carbamate

Tert-butyl(1-(4-chlorothiazol-2-yl)piperidin-4-yl)carbamate (600 mg, 1.89 mmol), (4-cyano-3-fluorophenyl)boronic acid (342 mg, 2.07 mmol), Cs₂CO₃ (1.6 g, 5.06 mmol), and Pd(dppf)Cl₂ (106 mg, 0.144 mmol) are dissolved in 14 mL of dioxane and added with 0.4 mL of water; and this mixed reaction solution is purged by nitrogen blowing, warmed to 120° C., and reacted for 0.5 h while stirring. When the reaction is completed, the reaction solution is concentrated under a reduced pressure; and the residue is purified by silica gel chromatography (eluent: petroleum ether/ethyl acetate=10/7) to obtain tert-butyl(1-(4-(4-cyano-3-fluorophenyl)thiazol-2-yl)piperidin-4-yl)carbamate with a yield of 80.3%.

ESI-MS m/z)=403.2 [M+H]⁺.

### Step c): Preparation of tert-butyl [1-(5-bromo-4-(4-cyano-3-fluorophenyl)thiazol-2-yl)piperidin-4-yl]carbamate

Tert-butyl(1-(4-(4-cyano-3-fluorophenyl)thiazol-2-yl)piperidin-4-yl) carbamate (300 mg, 0.744 mmol) is dissolved in N,N-dimethylformamide (10 mL), added with NBS (136 mg, 0.744 mmol) at 0° C. and reacted at room temperature. When LS-MS shows that the reaction is completed, the reaction solution is quenched with water (20 mL) and extracted with ethyl acetate (20 mL×2); organic layers are combined and then washed with saturated saline (15 mL×2), dried with anhydrous sodium sulfate, filtered, and concentrated; and the residue is purified by silica gel chromatography (eluent: petroleum ether/ethyl acetate=10/7) to obtain tert-butyl [1-(5-bromo-4-(4-cyano-3-fluorophenyl)thiazol-2-yl)piperidin-4-yl]carbamate with a yield of 50.5%.

ESI-MS m/z)=481.1 [M+H]⁺.

### Step d): Preparation of tert-butyl(1-(4-(4-cyano-3-fluorophenyl)-5-(3-hydroxy-4-methoxyphenyl)thiazol-2-yl)piperidin-4-yl)carbamate

Tert-butyl(1-(5-bromo-4-(4-cyano-3-fluorophenyl)thiazol-2-yl)piperidin-4-yl)carbamate (80 mg, 0.16 mmol), (3-hydroxy-4-methylphenyl)boronic acid (63 mg, 0.25 mmol), Cs₂CO₃ (162 mg, 0.5 mmol), and Pd(dppf)Cl₂ (10 mg, 0.016 mmol) are dissolved in 4 mL of dioxane and added with 0.4 mL of water; and this mixed reaction solution is purged by nitrogen blowing. The mixture reacts in a microwave reactor at 120° C. for 30 min; when LC-MS shows that the reaction is completed, a crude product is obtained by concentration, and the residue is purified by silica gel chromatography (eluent: dichloromethane/ethyl acetate=10/3) to obtain tert-butyl(1-(4-(4-cyano-3-fluorophenyl)-5-(3-hydroxy-4-methoxyphenyl)thiazol-2-yl)piperidin-4-yl)carbamate with a yield of 70.5%.

ESI-MSm/z=525.2[M+H]⁺

### Step e): Preparation of 4-(2-(4-aminopiperidin-1-yl)-5-(3-hydroxy-4-methoxyphenyl)thiazol-4-yl)-2-fluorobenzonitrile

Tert-butyl(1-(4-(4-cyano-3-fluorophenyl)-5-(3-hydroxy-4-methoxyphenyl)thiazol-2-yl)piperidin-4-yl)carbamate (60 mg, 0.114 mol) is added to 10 mL of 4M hydrochloric acid (ethyl acetate solution), and reacted for 2 h at room temperature, a small amount of solids are formed in the reaction system, and LC-MS shows that the reaction is completed. After the solvent is dried by spinning, the crude product is subjected to Prep-HPLC preparation (separation method 4) to obtain 4-(2-(4-aminopiperidin-1-yl)-5-(3-hydroxy-4-methoxyphenyl)thiazol-4-yl)-2-fluorobenzonitrile with a yield of 70.2%.

¹H NMR (400 MHz, DMSO-d₆) δ ppm 7.80 (dd, J=8.2, 7.2 Hz, 1H), 7.48 (dd, J=11.2, 1.6 Hz, 1H), 7.39 (dd, J=8.2, 1.6 Hz, 1H), 6.94 (d, J=8.2 Hz, 1H), 6.71 (d, J=7.4 Hz, 2H), 3.85 (dt, J=13.2, 4.0 Hz, 2H), 3.79 (s, 3H), 3.11 (ddd, J=13.4, 11.2, 3.0 Hz, 2H), 2.83 (tt, J=9.8, 3.8 Hz, 1H), 1.81 (dd, J=13.2, 3.6 Hz, 2H), 1.45-1.17 (m, 2H).

ESI-MS m/z: 425.1 [M+H]⁺.

Compounds in Examples 224-249 are prepared according to the synthesis method of Example 2 (separation method for the compounds: free alkali, hydrochloride and formate are separately prepared according to separation methods 4, 1 and 3, respectively), and their structures and characterization data are as follows:

| Ex- | | | | MS |
|---|---|---|---|---|
| amples | Chemical name | Structure | ¹H NMR | (M + H)+ |
| 224 | (6-(((1R,2S)-2-(4-fluoro phenyl)cyclopropyl) amino)methyl)-2-(3- hydroxy-4-methoxyphenyl) pyrimidin-4-yl)(4-(methyl- sulfonyl)piperazin-1-yl) methanone | 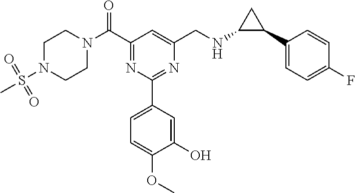 [SMILES: COc1ccc(-c2nc(CN[C@@H]3C[C@H]3c3ccc(F)cc3)cc(C(=O)N3CCN(S(C)(=O)=O)CC3)n2)cc1O] | ¹H NMR (400 MHz, Methanol-d₄) δ ppm 7.96 (dd, J = 8.6, 2.2 Hz, 1H), 7.91 (d, J = 2.0 Hz, 1H), 7.45 (s, 1H), 7.02 (dd, J = 8.6, 5.2 Hz, 3H), 6.97-6.84 (m, 2H), 4.08 (s, 2H), 4.02-3.86 (m, 5H), 3.79- 3.65 (m, 2H), 3.39 (t, J = 5.2 Hz, 2H),3.30 (s, 2H), 2.90 (s, 3H), 2.42 (ddd, J = 7.4, 4.4, 3.2 Hz, 1H), 1.97 (ddd, J = 9.4, 5.8, 3.2 Hz, 1H), 1.14 (ddd, J = 9.6, 5.4, 4.4 Hz, 1H), 0.99 (dt, J = 7.2, 5.6 Hz, 1H). | 556.2 |
| 225 | (2-(3-fluoro-4-methoxy phenyl)-6-(((1R,2S)-2- (4-fluorophenyl)cyclo- propyl)amino)methyl) pyrimidin-4-yl)(4-(methyl- sulfonyl)piperazin-1-yl) methanone | 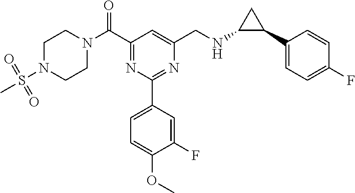 [SMILES: COc1ccc(-c2nc(CN[C@@H]3C[C@H]3c3ccc(F)cc3)cc(C(=O)N3CCN(S(C)(=O)=O)CC3)n2)cc1F] | ¹H NMR (400 MHz, Chloroform-d) δ ppm 8.28 (d, J = 8.8 Hz, 1H), 8.25-8.18 (m, 1H), 7.68 (s, 1H), 7.15-7.03 (m, 3H), 6.98 (t, J = 8.4 Hz, 2H), 4.36 (s, 2H), 4.03 (s, 3H), 4.00 (d, J = 5.6 Hz, 2H), 3.85 (s, 2H), 3.44 (d, J = 18.0 Hz, 4H), 2.92 (s, 3H), 2.75 (s, 1H), 2.54 (s, 1H), 1.32 (s, 1H), 1.17 (d, J = 6.6 Hz, 1H) | 558.2 |
| 226 | (2-(4-(1H-pyrazol-5-yl) phenyl)-6-(1R,2S)-2-(4- fluorophenyl)cyclopropyl) amino)methyl)pyrimidin- 4-yl)(4-(methylsulfonyl) piperazin-1-yl)methanone | 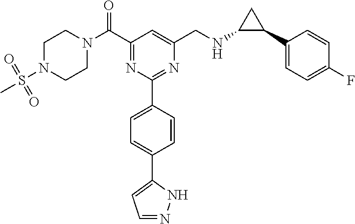 [SMILES: CS(=O)(=O)N1CCN(C(=O)c2cc(CN[C@@H]3C[C@H]3c3ccc(F)cc3)nc(-c3ccc(-c4ccn[nH]4)cc3)n2)CC1] | ¹H NMR (400 MHz, DMSO-d₆) δ ppm 13.01 (s, 1H), 8.40 (d, J = 8.0 Hz, 2H), 7.97 (d, J = 8.2 Hz, 2H), 7.83 (s, 1H), 7.62 (s, 1H), 7.16-6.91 (m, 4H), 6.82 (d, J = 2.2 Hz, 1H), 4.03 (s, 2H), 3.81 (t, J = 5.0 Hz, 2H), 3.61 (t, J = 5.2 Hz, 4H), 3.29 (s, 2H), 3.20 (t, J = 5.0 Hz, 2H), 2.95 (s, 3H), 2.38-2.30 (m, 1H), 1.92 (ddd, J = 9.0, 5.8, 3.0 Hz, 1H). | 576.2 |
| 227 | (6-((1R,2S)-2-(4-fluoro phenyl)cyclopropyl) amino)methyl)-2-(4- morpholinophenyl) pyrimidin-4-yl)(4- (methylsulfonyl) piperazin-1-yl) methanone | 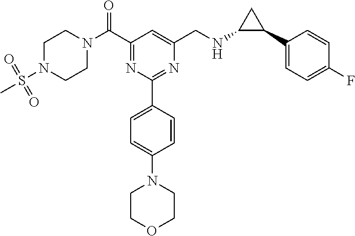 [SMILES: CS(=O)(=O)N1CCN(C(=O)c2cc(CN[C@@H]3C[C@H]3c3ccc(F)cc3)nc(-c3ccc(N4CCOCC4)cc3)n2)CC1] | ¹H NMR (400 MHz, Methanol-d₄) δ ppm 8.45-8.26 (m, 2H), 7.42 (s, 1H), 7.04 (dd, J = 8.8, 6.2 Hz, 4H), 7.00-6.85 (m, 2H), 4.59 (s, 2H), 4.09 (s, 2H), 3.92 (t, J = 5.2 Hz, 2H), 3.89-3.83 (m, 4H), 3.76- 3.66 (m, 2H), 3.40 (t, J = 5.2 Hz, 2H), 3.30 (t, J = 3.8 Hz, 4H), 2.92 (s, 3H), 2.43 (ddd, J = 7.4, 4.2, 3.2 Hz, 1H), 1.99 (ddd, J = 9.4, 5.8, 3.2 Hz, 1H), 1.16 (ddd, J = 9.6, 5.4, 4.2 Hz, 1H), 1.01 (dt, J = 7.4, 5.6 Hz, 1H). | 595.2 |
| 228 | (2-(4-(1H-1,2,3-triazol- 1-yl)phenyl)-6-((1R,2S)- 2-(4-fluorophenyl)cyclo- propyl)amino)methyl) pyrimidin-4-yl)(4- (methylsulfonyl) piperazin-1- yl)methanone | 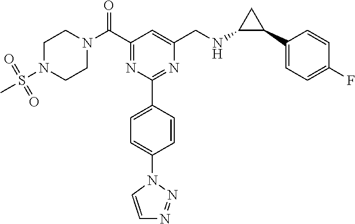 [SMILES: CS(=O)(=O)N1CCN(C(=O)c2cc(CN[C@@H]3C[C@H]3c3ccc(F)cc3)nc(-c3ccc(-n4ccnn4)cc3)n2)CC1] | ¹H NMR (400 MHz, Methanol-d₄) δ ppm 8.77-8.63 (m, 3H), 8.23- 8.01 (m, 2H), 7.97 (d, J = 1.2 Hz, 1H), 7.64 (s, 1H), 7.04 (ddd, J = 8.4, 5.4, 2.6 Hz, 2H), 7.00-6.80 (m, 2H), 4.18 (s, 2H), 4.05-3.86 (m, 2H), 3.74 (t, J = 5.2 Hz, 2H), 3.43 (t, J = 5.2 Hz, 2H), 3.33 (s, 2H), 2.93 (s, 3H), 2.46 (ddd, J = 7.4, 4.2, 3.2 Hz, 1H), 1.99 (ddd, J = 9.4, 5.8, 3.2 Hz, 1H), 1.27-1.11 (m, 1H), 1.03 (dt, J = 7.4, 5.6 Hz, 1H). | 577.2 |
| 229 | (2-(4-(1H-pyrazol-1-yl) phenyl)-6-(1R,2S)-2-(4- fluorophenyl)cyclopropyl) amino)methyl)pyrimidin- 4-yl)(4-morpholino) methanone | 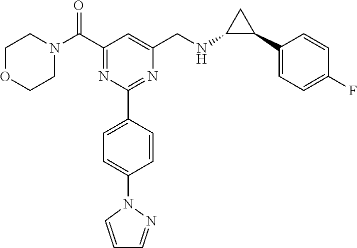 [SMILES: O=C(c1cc(CN[C@@H]2C[C@H]2c2ccc(F)cc2)nc(-c2ccc(-n3cccn3)cc2)n1)N1CCOCC1] | ¹H NMR (400 MHz, Methanol-d₄) δ ppm 8.69-8.48 (m, 2H), 8.34 (d, J = 2.4 Hz, 1H), 7.99-7.84 (m, 2H), 7.78 (d, J = 1.6 Hz, 1H), 7.55 (s, 1H), 7.13-6.99 (m, 2H), 6.98- 6.84 (m, 2H), 6.58 (t, J = 2.2 Hz, 1H), 4.13 (s, 2H), 3.82 (s, 4H), 3.71 (dd, J = 5.6, 4.0 Hz, 2H), 3.59 (dd, J = 5.6, 4.0 Hz, 2H), 2.43 (dt, J = 7.4, 3.8 Hz, 1H), 1.98 (ddd, J = 9.2, 5.8, 3.2 Hz, 1H), 1.15 (dt, J = 9.6, 4.8 Hz, 1H), 1.01 (dt, J = 7.2, 5.6 Hz, 1H). | 499.2 |
| 230 | (2-(4-(1H-pyrazol-1-yl) phenyl)-6-(1R,2S)-2-(4- fluorophenyl)cyclopropyl) amino)methyl)pyrimidine- 4-yl) (4,4-difluoropiperidin-1- yl)methanone | 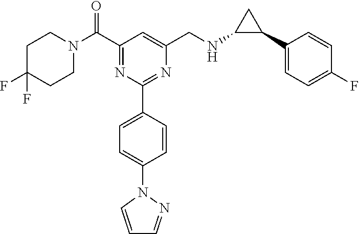 [SMILES: O=C(c1cc(CN[C@@H]2C[C@H]2c2ccc(F)cc2)nc(-c2ccc(-n3cccn3)cc2)n1)N1CCC(F)(F)CC1] | ¹H NMR (400 MHz, Methanol-d₄) δ ppm 8.69-8.49 (m, 2H), 8.36 (d, J = 2.6 Hz, 1H), 8.03-7.84 (m, 2H), 7.80 (d, J = 1.8 Hz, 1H), 7.58 (s, 1H), 7.12-6.99 (m, 2H), 6.99- 6.86 (m, 2H), 6.60 (s, 1H), 4.15 (s, 2H), 3.95 (t, J = 6.0 Hz, 2H), 3.68 (t, J = 5.8 Hz, 2H), 2.56-2.38 (m, 1H), 2.17 (qt, J = 13.4, 5.8 Hz, 4H), 2.00 (ddd, J = 9.4, 5.8, 3.2 Hz, 1H), 1.17 (dt, J = 9.6, 4.8 Hz, 1H), 1.03 (dt, J = 7.2, 5.6 Hz, 1H). | 533.2 |
| 231 | Methyl 1-(2-(4-(1H-pyrazol-1-yl) phenyl)-6-(1R,2S)-2-(4- fluorophenyl)cyclopropyl) amino)methylpyrimidine- 4-carbonyl)piperidine- 4-carboxylate | 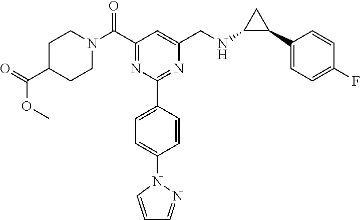 [SMILES: COC(=O)C1CCN(C(=O)c2cc(CN[C@@H]3C[C@H]3c3ccc(F)cc3)nc(-c3ccc(-n4cccn4)cc3)n2)CC1] | ¹H NMR (400 MHz, Methanol-d₄) δ ppm 8.59 (d, J = 8.8 Hz, 2H), 8.35 (d, J = 2.6 Hz, 1H), 7.91 (d, J = 8.8 Hz, 2H), 7.79 (d, J = 1.6 Hz, 1H), 7.52 (d, J = 1.2 Hz, 1H), 7.04 (dd, J = 8.4, 5.4 Hz, 2H), 6.92 (t, J = 8.8 Hz, 2H), 6.59 (s, 1H), 4.51 (dt, J = 13.2, 4.2 Hz, 1H), 4.14 (s, 2H), 3.91-3.77 (m, 1H), 3.73 (s, 3H), 3.22 (dddd, J = 37.8, 13.8, 11.2, 3.0 Hz, 2H), 2.77 (tt, J = 10.8, 4.0 Hz, 1H), 2.44 (qd, J = 4.4, 1.2 Hz, 1H), 2.17-2.04 (m, 1H), 2.04-1.90 (m, 2H), 1.80 (dt, J = | 555.2 |
| | | | 14.2, 11.0 Hz, 2H), 1.17 (dt, J = | |
| | | | 9.4, 4.8 Hz, 1H), 1.09-0.93 (m, 1H). | |
| 232 | (6-((1R,2S)-2-(4-fluoro phenyl)cyclopropyl) amino)methyl)-2-(imidazolo [1,2-a]pyridin-7-yl) pyrimidin-4-yl)(4-(methyl- sulfonyl)piperazin-1-yl) methanone | 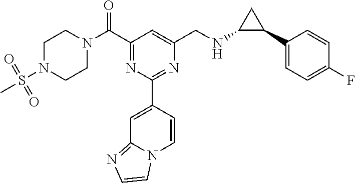 [SMILES: CS(=O)(=O)N1CCN(C(=O)c2cc(CN[C@@H]3C[C@H]3c3ccc(F)cc3)nc(-c3ccn4ccnc4c3)n2)CC1] | ¹H NMR (400 MHz, Methanol-d₄) δ ppm 8.71 (s, 1H), 8.56 (d, J = 7.0 Hz, 1H), 8.13-7.92 (m, 2H), 7.74 (d, J = 1.2 Hz, 1H), 7.65 (s, 1H), 7.15-6.99 (m, 2H), 6.99- 6.84 (m, 2H), 4.19 (s, 2H), 3.96 (t, J = 5.0 Hz, 2H), 3.78-3.69 (m, 2H), 3.44 (t, J = 5.2 Hz, 2H), 3.38 (t, J = 5.0 Hz, 2H), 2.95 (s, 3H), 2.47 (dt, J = 7.4, 3.8 Hz, 1H), 1.99 (ddd, J = 9.2, 5.8, | 550.2 |
| | | | 3.2 Hz, 1H), 1.18 (dt, J = 9.6, | |
| | | | 4.8 Hz, 1H), 1.04 (dt, J = 7.2, | |
| | | | 5.8 Hz, 1H). | |
| 233 | 2-(4-(1H-pyrazol-1-yl) phenyl)-6-(((1R,2S)-2-(4- fluorophenyl)cyclopropyl) amino)methyl)-N- (tetrahydro-2H-pyran-4- yl)pyrimidine-4- carboxamide | 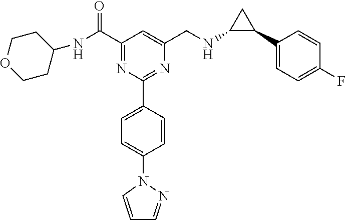 [SMILES: O=C(NC1CCOCC1)c1cc(CN[C@@H]2C[C@H]2c2ccc(F)cc2)nc(-c2ccc(-n3cccn3)cc2)n1] | ¹H NMR (400 MHz, Methanol-d₄) δ ppm 8.76-8.53 (m, 2H), 8.24 (d, J = 2.6 Hz, 1H), 7.95-7.74 (m, 3H), 7.68 (d, J = 1.8 Hz, 1H), 6.89 (ddd, J = 8.6, 5.4, 2.6 Hz, 2H), 6.87-6.74 (m, 2H), 6.48 (t, J = 2.2 Hz, 1H), 4.22-4.00 (m, 3H), 3.93 (ddd, J = 11.9, 4.4, 2.2 Hz, 2H), 3.47 (td, J = 11.7, 2.4 Hz, 2H), 2.32 (ddd, J = 7.4, 4.4, 3.2 Hz, 1H), 1.94-1.61 (m, 5H), 1.05 (ddd, J = 9.6, 5.3, 4.4 Hz, 1H), 0.90 (dt, J = 7.2, 5.6 Hz, 1H). | 513.2 |
| 234 | 1-(2-(4-(1H-pyrazol-1-yl) phenyl)-6-(((1R,2S)-2- (4-fluorophenyl)cyclo- propyl)amino)methyl) pyrimidine-4-carbonyl) piperidine-4-carboxylic acid | 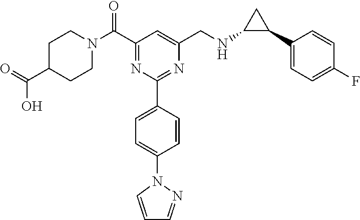 [SMILES: O=C(O)C1CCN(C(=O)c2cc(CN[C@@H]3C[C@H]3c3ccc(F)cc3)nc(-c3ccc(-n4cccn4)cc3)n2)CC1] | ¹H NMR (400 MHz, Methanol-d₄) δ ppm 8.81-8.48 (m, 2H), 8.35 (d, J = 2.6 Hz, 1H), 8.02-7.85 (m, 2H), 7.79 (d, J = 1.8 Hz, 1H), 7.52 (d, J = 2.8 Hz, 1H), 7.04 (ddd, J = 8.4, 5.4, 2.6 Hz, 2H), 6.98-6.86 (m, 2H), 6.59 (dd, J = 2.6, 1.8 Hz, 1H), 4.68-4.43 (m, 1H), 4.15 (s, 2H), 3.81 (tdd, J = 10.8, 9.4, 4.4, 2.0 Hz, 1H), 3.23 (tdd, J = 10.8, 5.0, 2.6 Hz, 1H), 3.19-3.04 (m, 1H), 2.56 (dddd, J = 13.6, 9.4, 4.2, 2.8 Hz, 1H), 2.46 (tdd, J = 7.2, 4.4, 3.2 Hz, 1H), 2.17-2.06 (m, 1H), | 541.2 |
| | | | 2.06-1.98 (m, 1H), 1.98-1.89 | |
| | | | (m, 1H), 1.89-1.76 (m, 2H), 1.18 | |
| | | | (dddd, J = 9.6, 5.4, 4.2, 1.1 Hz, | |
| | | | 1H), 1.07-0.89 (m, 1H). | |
| 235 | (2-(5-fluoro-3-methyl- benzo[d]isoxazol-6-yl)-6- (((1R,2S)-2-(4-fluoro- phenyl)cyclopropyl)amino) methyl)pyrimidin-4-yl) (4-(methylsulfonyl) piperazin-1-yl)methanone | 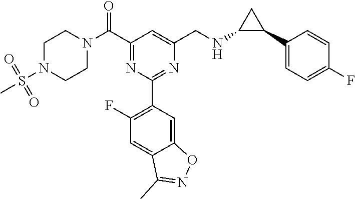 [SMILES: Cc1noc2cc(-c3nc(CN[C@@H]4C[C@H]4c4ccc(F)cc4)cc(C(=O)N4CCN(S(C)(=O)=O)CC4)n3)c(F)cc12] | ¹H NMR (400 MHz, Methanol-d₄) δ ppm 8.21 (d, J = 5.6 Hz, 1H), 7.88-7.45 (m, 2H), 7.03 (dd, J = 8.6, 5.4 Hz, 2H), 6.91 (t, J = 8.8 Hz, 2H), 4.18 (d, J = 1.2 Hz, 2H), 3.93 (t, J = 5.0 Hz, 2H), 3.77 (t, J = 5.0 Hz, 2H), 3.40 (t, J = 5.2 Hz, 2H), 3.36 (s, 2H), 2.91 (s, 3H), 2.63 (s, 3H), 2.45 (dt, J = 7.4, 3.8 Hz, 1H), 1.96 (ddd, J = 9.2, 5.8, 3.2 Hz, 1H), 1.16 (dt, J = 9.4, 4.8 Hz, 1H), 1.02 (dt, J = 7.2, 5.6 Hz, 1H). | 583.2 |
| 236 | 6-(((1R,2S)-2-(4-fluoro phenyl)cyclopropyl)amino) methyl)-2-(3-hydroxy- 4-methylphenyl)-N- (tetrahydro- 2H-pyran-4-yl)pyrimidine- 4-carboxamide | 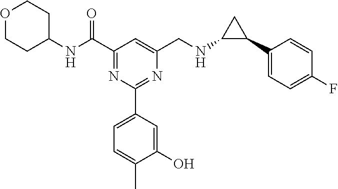 [SMILES: Cc1ccc(-c2nc(CN[C@@H]3C[C@H]3c3ccc(F)cc3)cc(C(=O)NC3CCOCC3)n2)cc1O] | ¹H NMR (400 MHz, DMSO-d₆) δ ppm 9.49 (s, 1H), 8.71 (d, J = 8.4 Hz, 1H), 8.08-7.87 (m, 3H), 7.22 (d, J = 7.8 Hz, 1H), 7.01 (qd, J = 8.8, 6.2 Hz, 4H), 4.08 (q, J = 7.6 Hz, 1H), 4.01 (s, 2H), 3.91 (dt, J = 11.4, 3.4 Hz, 2H), 3.48-3.37 (m, 2H), 2.28 (dd, J = 7.2, 3.8 Hz, 1H), 2.20 (s, 3H), 1.90 (ddd, J = 9.2, 5.8, 3.0 Hz, 1H), 1.77 (td, J = 10.2, 8.8, 4.0 Hz, 4H), 1.04 (dt, J = 9.2, 4.6 Hz, 1H), 0.95 (dt, J = 7.2, 5.4 Hz, 1H). | 477.2 |
| 237 | 6-(((1R,2S)-2-(4-fluoro phenyl)cyclopropyl) amino)methyl)-2-phenyl-N- (tetrahydro-2H-pyran-4- yl)pyrimidine-4- carboxamide | 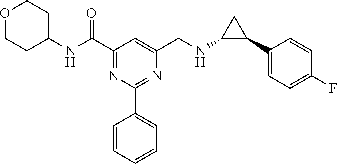 [SMILES: O=C(NC1CCOCC1)c1cc(CN[C@@H]2C[C@H]2c2ccc(F)cc2)nc(-c2ccccc2)n1] | ¹H NMR (400 MHz, DMSO-d₆) δ ppm 8.84 (d, J = 8.6 Hz, 1H), 8.62- 8.49 (m, 2H), 7.99 (s, 1H), 7.58- 7.44 (m, 3H), 7.06-6.94 (m, 4H), 4.11 (ddd, J = 13.8, 8.8, 4.8 Hz, 1H), 4.04 (s, 2H), 3.92 (dt, J = 11.4, 3.4 Hz, 2H), 3.42 (td, J = 11.4, 3.2 Hz, 2H), 2.29 (dt, J = 7.4, 3.8 Hz, 1H), 1.89 (ddt, J = 8.4, 5.6, 2.8 Hz, 1H), 1.79 (td, J = 11.8, 7.6 Hz, 4H), 1.05 (dt, J = 9.4, 4.8 Hz, | 447.2 |
| | | | 1H), 0.95 (dt, J = 7.2, 5.4 Hz, 1H). | |
| 238 | (2-(4-fluoro-3-hydroxy phenyl)-6-(((1R,2S)-2- (4-fluorophenyl)cyclo- propyl)amino)methyl) pyrimidin-4-yl)(4-(methyl- sulfonyl)piperazin-1-yl) methanone hydrochloride | 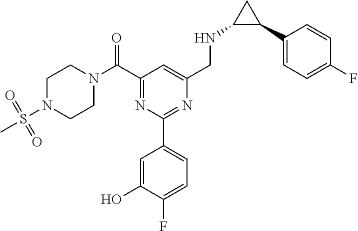 [SMILES: CS(=O)(=O)N1CCN(C(=O)c2cc(CN[C@@H]3C[C@H]3c3ccc(F)cc3)nc(-c3ccc(F)c(O)c3)n2)CC1] | ¹H NMR (400 MHz, DMSO-d₆) δ ppm 10.36-9.89 (m, 3H), 8.12 (dt, J = 10.0, 3.6 Hz, 1H), 8.01 (ddd, J = 8.6, 4.6, 2.2 Hz, 1H), 7.82-7.65 (m, 1H), 7.33 (ddt, J = 10.8, 8.6, 3.8 Hz, 1H), 7.22 (ddd, J = 8.8, 5.6, 2.8 Hz, 2H), 7.12 (td, J = 8.8, 2.2 Hz, 2H), 4.64 (s, 2H), 3.81 (t, J = 5.2 Hz, 2H), 3.59 (t, J = 4.8 Hz, 4H), 3.25 (dt, J = 25.2, 5.2 Hz, 2H), 3.09 (s, 1H), 2.97 (d, J = 1.6 Hz, 3H), 2.73-2.55 (m, 1H), 1.66 (d, J = 12.0 Hz, 1H), 1.33 (q, J = 6.6 Hz, 1H). | 544.2 |
| 239 | 4-(4-(3,5-dimethyl- piperazine-1-carbonyl)-6- ((((1R,2S)-2-(4-fluoro- phenyl)cyclopropyl)amino) methyl)pyrimidin-2-yl) benzonitrile | 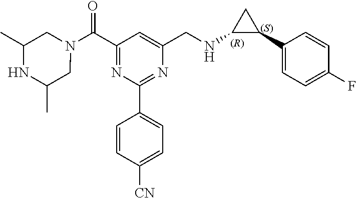 | ¹H NMR (400 MHz, DMSO-d₆) δ ppm 8.49 (d, J = 8.2 Hz, 2H), 8.11- 7.86 (m, 2H), 7.65 (s, 1H), 7.07- 6.89 (m, 4H), 4.45-4.32 (m, 1H), 4.03 (s, 2H), 3.49 (d, J = 12.7 Hz, 1H), 2.75 (s, 2H), 2.72-2.57 (m, 1H), 2.39-2.22 (m, 2H), 1.87 (ddt, J = 8.8, 5.6, 2.6 Hz, 1H), 1.04 (dd, J = 5.6, 3.8 Hz, 4H), 0.95 (q, J = 5.8 Hz, 1H), 0.83 (dd, J = 6.2, 2.2 Hz, 3H). | 485.2 |
| 240 | (6-((1R,2S)-2-(4-fluoro phenyl)cyclopropyl) amino)methyl)-2-(pyridin- 3-yl)pyrimidin-4-yl) (morpholino) methanone | 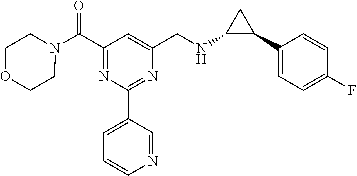 [SMILES: O=C(c1cc(CN[C@@H]2C[C@H]2c2ccc(F)cc2)nc(-c2cccnc2)n1)N1CCOCC1] | ¹H NMR (400 MHz, Methanol-d₄) δ ppm 9.56 (dd, J = 2.2, 0.8 Hz, 1H), 8.81 (dt, J = 8.2, 1.8 Hz, 1H), 8.69 (dd, J = 5.0, 1.8 Hz, 1H), 7.68- 7.55 (m, 2H), 7.01 (ddd, J = 8.6, 5.4, 2.6 Hz, 2H), 6.96-6.85 (m, 2H), 4.23-4.10 (m, 2H), 3.83 (s, 4H), 3.72 (dd, J = 5.6, 3.8 Hz, 2H), 3.59 (dd, J = 5.6, 3.8 Hz, 2H), 2.44 (ddd, J = 7.4, 4.4, 3.2 Hz, 1H), 1.96 (ddd, J = 9.4, 5.8, 3.2 Hz, 1H), 1.16 | 434.2 |
| | | | (ddd, J = 9.6, 5.4, 4.4 Hz, 1H), 1.02 | |
| | | | (dt, J = 7.2, 5.6 Hz, 1H). | |
| 241 | (6-(((1R,2S)-2-(4-fluoro phenyl)cyclopropyl) amino)methyl)-2-(3- hydroxy-4-methoxyphenyl) pyrimidin-4-yl) (morpholino)methanone | 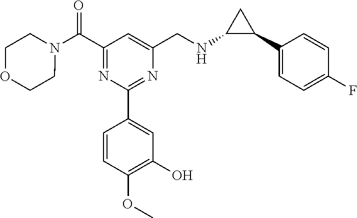 [SMILES: COc1ccc(-c2nc(CN[C@@H]3C[C@H]3c3ccc(F)cc3)cc(C(=O)N3CCOCC3)n2)cc1O] | ¹H NMR (400 MHz, Methanol-d₄) δ ppm 7.89-7.70 (m, 2H), 7.30 (s, 1H), 7.02-6.84 (m, 3H), 6.84- 6.66 (m, 2H), 3.95 (s, 2H), 3.80 (s, 3H), 3.67 (s, 4H), 3.58 (dd, J = 5.6, 3.8 Hz, 2H), 3.44 (dd, J = 5.6, 4.0 Hz, 2H), 2.28 (ddd, J = 7.4, 4.2, 3.2 Hz, 1H), 1.84 (ddd, J = 9.2, 5.8, 3.2 Hz, 1H), 1.01 (ddd, J = 9.6, 5.4, 4.2 Hz, 1H), 0.86 (dt, J = 7.2, 5.6 Hz, 1H). | 479.2 |
| 242 | (2-(4-(1H-1,2,3-triazol- 1-yl)phenyl)-6-(((1R,2S)- 2-(4-fluorophenyl)cyclo- propyl)amino)methyl) pyrimidin-4-yl) (morpholino)methanone | 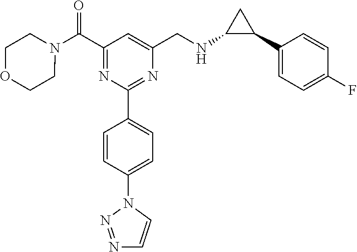 [SMILES: O=C(c1cc(CN[C@@H]2C[C@H]2c2ccc(F)cc2)nc(-c2ccc(-n3ccnn3)cc2)n1)N1CCOCC1] | ¹H NMR (400 MHz, Methanol-d₄) δ ppm 8.81-8.37 (m, 3H), 8.17- 7.98 (m, 2H), 7.94 (d, J = 1.2 Hz, 1H), 7.60 (s, 1H), 7.01 (ddd, J = 8.4, 5.4, 2.6 Hz, 2H), 6.98-6.80 (m, 2H), 4.15 (s, 2H), 3.82 (s, 4H), 3.72 (dd, J = 5.6, 4.2 Hz, 2H), 3.59 (dd, J = 5.6, 3.8 Hz, 2H), 2.43 (ddd, J = 7.4, 4.2, 3.2 Hz, 1H), 1.97 (ddd, J = 9.2, 5.8, 3.4 Hz, 1H), 1.16 (ddd, J = 9.6, 5.4, 4.2 Hz, 1H), 1.01 (dt, J = 7.2, 5.6 Hz, 1H). | 500.2 |
| 243 | 6-(((1R,2S)-2-(4-fluoro phenyl)cyclopropyl) amino)-2-(3-hydroxy-4- methoxyphenyl)-n-(tetra- hydro-2H pyran-4-yl)pyrimidine- 4-carboxamide | 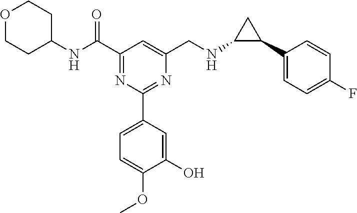 [SMILES: COc1ccc(-c2nc(CN[C@@H]3C[C@H]3c3ccc(F)cc3)cc(C(=O)NC3CCOCC3)n2)cc1O] | ¹H NMR (400 MHz, DMSO-d₆) δ ppm 8.79 (d, J = 8.4 Hz, 1H), 8.15- 8.04 (m, 2H), 7.87 (s, 1H), 7.01 (dd, J = 17.8, 8.8, 6.2, 2.4 Hz, 4H), 6.91 (d, J = 8.2 Hz, 1H), 4.09 (dt, J = 14.8, 7.6 Hz, 1H), 4.00 (s, 2H), 3.91 (dt, J = 11.2, 3.4 Hz, 2H), 3.87 (s, 3H), 3.42 (ddd, J = 14.2, 9.6, 4.2 Hz, 2H), 2.35-2.25 (m, 1H), 1.90 (ddd, J = 9.2, 5.8, 3.0 Hz, 1H), 1.78 (td, J = 9.4, 8.4, 3.8 Hz, 4H), 1.05 (dt, J = 9.2, 4.6 Hz, 1H), 0.94 (dt, J = 6.8, 5.2 Hz, 1H). | 493.2 |
| 244 | (4,4-difluoropiperidin-1- yl)(6-(((1R,2S)-2-(4- f1uorophenyl)cyclopropyl) amino)methyl)-2-(4- morpholinophenyl) pyrimidin-4-yl)methanone hydrochloride | 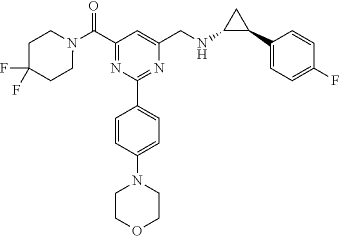 [SMILES: O=C(c1cc(CN[C@@H]2C[C@H]2c2ccc(F)cc2)nc(-c2ccc(N3CCOCC3)cc2)n1)N1CCC(F)(F)CC1] | ¹H NMR (400 MHz, DMSO-d₆) δ 9.93 (d, J = 27.2 Hz, 1H), 8.37 (d, J = 9.0 Hz, 2H), 7.57 (s, 1H), 7.26- 7.19 (m, 2H), 7.17-7.04 (m, 4H), 4.64 (s, 2H), 3.87-3.69 (m, 6H), 3.63-3.46 (m, 2H), 3.29 (t, J = 5.0 Hz, 4H), 3.10 (dt, J = 8.0, 4.2 Hz, 1H), 2.60 (ddd, J = 10.2, 6.5, 3.6 Hz, 1H), 2.12 (dt, J = 13.6, 6.6 Hz, 4H), 1.62 (ddd, J = 10.4, 6.3, 4.4 Hz, 1H), 1.35 (q, J = 6.8 Hz, 1H). | 552.2 |
| 245 | (6-((1R,2S)-2-(4-fluoro phenyl)cyclopropyl) amino)methyl)-2-(4- morpholinophenyl) pyrimidin-4-yl) (morpholino)methanone hydrochloride | 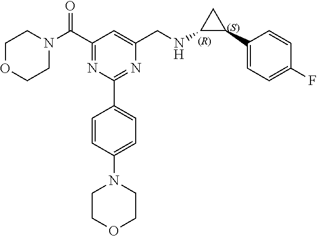 | ¹H NMR (400 MHz, DMSO-d₆) δ 10.07 (br,2H), 8.36 (d, J = 9.0 Hz, 2H), 7.56 (s, 1H), 7.25-7.19 (m, 2H), 7.17-7.04 (m, 4H), 4.62 (s, 2H), 3.76 (dd, J = 6.0, 3.8 Hz, 4H), 3.70 (d, J = 2.8 Hz, 4H), 3.59 (s, 2H), 3.46 (t, J = 4.8 Hz, 2H), 3.29 (t, J = 4.8 Hz, 4H), 3.09 (dt, J = 8.2, 4.2 Hz, 1H), 2.70-2.56 (m, 1H), 1.64 (ddd, J = 10.6, 6.3, 4.4 Hz, 1H), 1.34 (q, J = 6.8 Hz, 1H). | 518.2 |
| 246 | 6-(((1R,2S)-2-(4-fluoro phenyl)cyclopropyl)ami no)methyl)-2-(4-morpho- linophenyl)-N-(tetrahydro- 2H-pyran-4-yl)pyrimidine- 4-carboxamide hydrochloride | 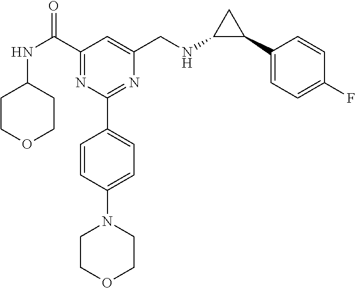 [SMILES: O=C(NC1CCOCC1)c1cc(CN[C@@H]2C[C@H]2c2ccc(F)cc2)nc(-c2ccc(N3CCOCC3)cc2)n1] | ¹H NMR (400 MHz, DMSO-d₆) δ 9.97 (br,2H), 8.90 (d, J = 8.5 Hz, 1H), 8.60 (d, J = 9.0 Hz, 2H), 7.91 (s, 1H), 7.22 (qd, J = 5.7, 2.2 Hz, 2H), 7.17-7.03 (m, 4H), 4.69 (s, 2H), 4.09 (tt, J = 10.8, 4.7 Hz, 1H), 3.97-3.87 (m, 2H), 3.78 (t, J = 4.8 Hz, 4H), 3.42 (td, J = 13.1, 11.5, 4.0 Hz, 2H), 3.31 (t, J = 4.9 Hz, 4H), 3.07 (dt, J = 8.3, 4.2 Hz, 1H), 2.33 (p, J = 1.9 Hz, 1H), 1.80 (ddt, J = 20.2, 15.3, 7.5 Hz, 4H), 1.61 (ddd, J = 10.5, 6.3, 4.4 Hz, 1H), 1.42-1.30 (m, 1H). | 532.3 |
| 247 | (4,4-difluoropiperidin-1- yl) (6-(1R,2S)-2-(4-fluoro- phenyl)cyclopropyl) amino)methyl)-2-(3- hydroxy-4-methoxyphenyl) pyrimidin-4-yl)methanone | 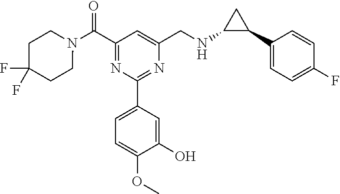 [SMILES: COc1ccc(-c2nc(CN[C@@H]3C[C@H]3c3ccc(F)cc3)cc(C(=O)N3CCC(F)(F)CC3)n2)cc1O] | ¹H NMR (400 MHz, DMSO-d₆) δ 7.83 (d, J = 8.2 Hz, 2H), 7.54 (s, 1H), 7.09-6.95 (m, 5H), 3.97 (s, 2H), 3.84 (s, 3H), 3.79 (t, J = 6.2 Hz, 2H), 3.53 (t, J = 6.0 Hz, 2H), 2.31 (ddd, J = 7.2, 4.2, 3.0 Hz, 1H), 2.10 (p, J = 14.0 Hz, 4H), 1.91 (ddd, J = 9.2, 5.8, 3.0 Hz, 1H), 1.04 (dt, J = 9.2, 4.8 Hz, 1H), 0.95 (dt, J = 7.0, 5.2 Hz, 1H). | 513.2 |
| 248 | (2-(4-(1H-1,2,3-triazol- 1-yl)phenyl)-6-((((1R,2S)- 2-(4-fluorophenyl)cyclo- propyl)amino)methyl) pyrimidin-4-yl)(4,4- difluoropiperidin-1-yl) methanone | 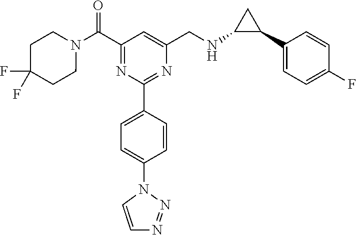 [SMILES: O=C(c1cc(CN[C@@H]2C[C@H]2c2ccc(F)cc2)nc(-c2ccc(-n3ccnn3)cc2)n1)N1CCC(F)(F)CC1] | ¹H NMR (400 MHz, DMSO-d₆) δ 8.95 (d, J = 1.2 Hz, 1H), 8.60- 8.52 (m, 2H), 8.14-8.08 (m, 2H), 8.03 (d, J = 1.2 Hz, 1H), 7.71 (s, 1H), 7.08-6.95 (m, 4H), 4.06 (s, 2H), 3.81 (t, J = 6.2 Hz, 2H), 3.56 (t, J = 6.0 Hz, 2H), 2.38-2.28 (m, 1H), 2.13 (dt, J = 13.4, 6.4 Hz, 4H), 1.92 (s, 1H), 1.15-1.03 (m, 1H), 0.97 (d, J = 6.4 Hz, 1H). | 534.2 |
| 249 | 2-(4-(1H-1,2,3-triazol-1- yl)phenyl)-6-((1R,2S)- 2-(4-fluorophenyl)cyclo propyl)amino)-n-(tetra- hydro-2H-pyran-4-yl) pyrimidin-4-carboxamide | 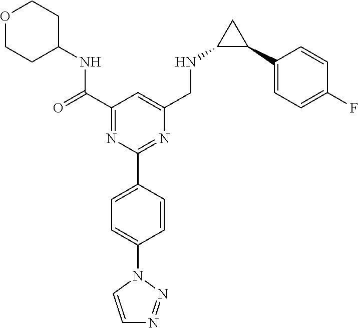 [SMILES: O=C(NC1CCOCC1)c1cc(CN[C@@H]2C[C@H]2c2ccc(F)cc2)nc(-c2ccc(-n3ccnn3)cc2)n1] | ¹H NMR (400 MHz, DMSO-d₆) δ ppm 9.02-8.89 (m, 2H), 8.84-8.73 (m, 2H), 8.15-7.91 (m, 4H), 7.08-6.85 (m, 4H), 4.21-4.09 (m, 1H), 4.07 (s, 2H), 3.93 (dt, J = 11.2, 3.4 Hz, 2H), 3.43 (td, J = 11.4, 2.8 Hz, 3H), 2.30 (dt, J = 7.4, 4.0 Hz, 1H), 1.94-1.73 (m, 4H), 1.10-0.92 (m, 2H). | 514.2 |

### Example 250

### Preparation of (4-(((1R,2 S)-2-(4-fluorophenyl)cyclopropyl)amino)methyl)-6-phenyl-1,3,5-triazin-2-yl)(4-(meth ylsulfonyl)piperazin-1-yl)methanone

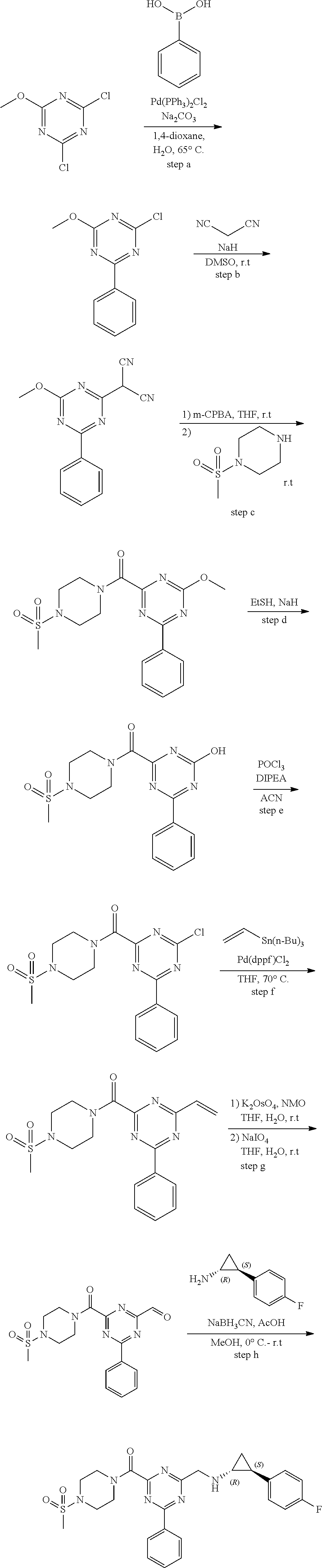

### Step a): Preparation of 2-chloro-4-methoxy-6-phenyl-1,3,5-triazine

2,4-dichloro-6-methoxy-1,3,5-triazine (5.0 g, 25.0 mmol), phenylboronic acid (4.6 g, 38.0 mmol), Pd(dpp₃)₂ (1.8 g, 2.5 mmol), Cs₂CO₃ (18.0 g, 56.0 mmol), 1,4-dioxane (50 ml) and water (12 mL) are sequentially added to a reaction flask, subjected to nitrogen displacement for three times, warmed to 65° C., and reacted for 16 h while stirring. When the reaction is completed, the reaction solution is concentrated under a reduced pressure; and the residue is purified by silica gel chromatography (eluent: petroleum ether/ethyl acetate=2/1) to obtain 2-chloro-4-methoxy-6-phenyl-1,3,5-triazine with a yield of 54.2%.

ESI-MS m/z)=222.1 [M+H]⁺.

### Step b): Preparation of 2-(4-methoxy-6-phenyl-1,3,5-triazin-2-yl)malononitrile

Malononitrile (3.3 g, 50.0 mmol) and DMSO (20 mL) are added to a reaction flask, added with NaH (1.0 g, 50.0 mmol, 60%) under ice bath agitation, and reacted at room temperature for 30 min while stirring. 2-chloro-4-methoxy-6-phenyl-1,3,5-triazine (5.5 g, 25.0 mmol) is added under ice bath agitation, and reacted for 30 min at room temperature while stirring. When the reaction is completed, the reaction solution is quenched with water (100 mL), and extracted with ethyl acetate (100 mL×3); organic phase is combined, and washed with saturated saline (100 mL×2), and concentrated under a reduced pressure to dryness; and the residue is pulped and purified by petroleum ether/ethyl acetate=3/1 (100 mL) and filtered to obtain 2-(4-methoxy-6-phenyl-1,3,5-triazin-2-yl)malononitrile with a yield of 61.0%.

ESI-MS(m/z)=252.1[M+H]⁻

### Step c): Preparation of (4-methoxy-6-phenyl-1,3,5-triazin-2-yl)(4-(methylsulfonyl)piperazin-1-yl)methanone

2-(4-methoxy-6-phenyl-1,3,5-triazin-2-yl)malononitrile (3.8 g, 15.25 mmol), m-CPBA (7.9 g, 45.75 mmol, 82%) and THF (40 mL) are added to a reaction flask, stirred at room temperature for 35 min, then added with 1-(methylsulfonyl)piperazine (7.5 g, 45.75 mmol), and maintained to react for 1 h at room temperature. When the reaction is completed, the reaction solution is quenched with water (50 mL), and extracted with ethyl acetate (50 mL×2); organic phase is combined, sequentially washed with a saturated sodium bicarbonate aqueous solution (50 mL) and saturated saline (50 mL×2), dried with anhydrous sodium sulfate, and filtered to obtain filtrate; the filtrate is concentrated under a reduced pressure; and the residue is purified by silica gel chromatography (eluent: petroleum ether/ethyl acetate=1: 1) to obtain (4-methoxy-6-phenyl-1,3,5-triazin-2-yl) (4-(methylsulfonyl)piperazin-1-yl)methanone with a yield of 15.5%.

ESI-MS m/z)=378.1 [M+H]⁺.

### Step d): Preparation of 4-hydroxy-6-phenyl-1,3,5-triazin-2-yl)(4-(methylsulfonyl)piperazin-1-yl)methanone

Ethanethiol (219 mg, 3.54 mmol) and DMF (10 mL) are added to a reaction flask, added with NaH (142 mg, 3.54 mmol, 60%) under ice bath agitation, and reacted while stirring at room temperature for 30 min. (4-methoxy-6-phenyl-1,3,5-triazin-2-yl) (4-(methylsulfonyl)piperazin-1-yl)methanone (884 mg, 2.36 mmol) is added under ice bath agitation, and reacted for 30 min at room temperature while stirring. When the reaction is completed, the reaction solution is quenched with water (100 mL), and extracted with ethyl acetate (100 mL×3); organic phase is combined, washed with saturated saline (100 mL×2), dried with anhydrous sodium sulfate, and filtered to obtain filtrate; and the filtrate is concentrated under a reduced pressure to obtain 4-hydroxy-6-phenyl-1,3,5-triazin-2-yl)(4-(methylsulfonyl)piperazin-1-yl)methanone with a yield of 53.0%.

¹H NMR (400 MHz, DMSO-d₆) δppm 13.40 (s, 1H), 8.38-8.14 (m, 2H), 7.69 (td, J=7.2, 1.4 Hz, 1H), 7.58 (t, J=7.8 Hz, 2H), 3.73 (t, J=5.2 Hz, 2H), 3.60 (t, J=5.2 Hz, 2H), 3.24 (t, J=5.2 Hz, 2H), 3.15 (t, J=5.2 Hz, 2H), 2.95 (s, 3H).

ESI-MS m/z)=364.1 [M+H]⁺.

### Step e): Preparation of (4-chloro-6-phenyl-1,3,5-triazin-2-yl) (4-(methylsulfonyl)piperazin-1-yl)methanone

(4-hydroxy-6-phenyl-1,3,5-triazin-2-yl)(4-(methylsulfonyl)piperazin-1-yl)methanone (455 mg, 1.25 mmol), phosphorus oxychloride (288 mg, 1.88 mmol), DIPEA (243 mg, 1.88 mmol) and acetonitrile (10 mL) are added sequentially to a reaction flask and stirred for 4 h at 60° C. When the reaction is completed, the reaction solution is concentrated under a reduced pressure; and the residue is purified by silica gel chromatography (eluent: petroleum ether/ethyl acetate=2/3) to obtain (4-chloro-6-phenyl-1,3,5-triazin-2-yl) (4-(methylsulfonyl)piperazin-1-yl)methanone with a yield of 75.3%.

ESI-MS m/z)=382.1 [M+H]⁺.

### Step f): Preparation of (4-(methylsulfonyl)piperazin-1-yl) (4-phenyl-6-vinyl-1,3,5-triazin-2-yl)methanone

(4-chloro-6-phenyl-1,3,5-triazin-2-yl) (4-(methylsulfonyl)piperazin-1-yl)methanone (341 mg, 0.94 mmol), vinyl tri-tert-butyltin (444 mg, 1.41 mmol), Pd(dppf)Cl₂ (68 mg, 0.09 mmol) and THE (10 ml) are added sequentially to a sealed-tube reactor, subjected to nitrogen displacement for three times, warmed to 70° C., and reacted for 16 h while stirring. When the reaction is completed, the reaction solution is concentrated under a reduced pressure; and the residue is purified by silica gel chromatography (eluent: petroleum ether/ethyl acetate=1/2) to obtain (4-(methylsulfonyl)piperazin-1-yl) (4-phenyl-6-vinyl-1,3,5-triazin-2-yl)methanone with a yield of 40.5%.

¹H NMR (400 MHz, DMSO-d₆) δ ppm 8.63-8.44 (m, 2H), 7.71 (t, J=7.4 Hz, 1H), 7.64 (s, 1H), 7.07-6.83 (m, 2H), 6.15 (dd, J=10.2, 1.8 Hz, 2H), 3.80 (t, J=5.0 Hz, 2H), 3.51 (t, J=4.8 Hz, 2H), 3.28 (d, J=6.0 Hz, 2H), 3.13 (t, J=5.0 Hz, 2H), 2.95 (d, J=6.2 Hz, 3H).

ESI-MS(m/z)=374.1 [M+H]⁺.

### Step g): Preparation of 4-(4-(methylsulfonyl)piperazine-1-carbonyl)-6-phenyl-1,3,5-triazine-2-carbaldehyde

4-(methylsulfonyl)piperazin-1-yl)(4-phenyl-6-vinyl-1,3,5-triazin-2-yl)methanone (142 mg, 0.38 mmol), NMO (133 uL, 0.57 mmol, 50%), potassium osmate (14 mg, 0.04 mmol), THE (6 mL) and water (2 mL) are added to a reaction flask and reacted for 1 h while stirring. When the reaction is completed, the reaction solution is added with sodium periodate (244 mg, 1.14 mmol), stirred for 1 h at room temperature till the reaction is completed, then quenched with water (20 mL), and extracted with ethyl acetate (20 mL×3); organic phase is combined, washed with saturated saline (20 mL×2), dried with anhydrous sodium sulfate, and filtered to obtain filtrate; the filtrate is concentrated under a reduced pressure; and the residue is purified by silica gel chromatography (eluent: petroleum ether/ethyl acetate=1/2) to obtain 4-(4-(methylsulfonyl)piperazine-1-carbonyl)-6-phenyl-1,3,5-triazine-2-carbaldehyde with a yield of 53.0%.

ESI-MS m/z)=376.1 [M+H]⁺.

### Step h): Preparation of (4-(((1R,2S)-2-(4-fluorophenyl)cyclopropyl)amino)methyl)-6-phenyl-1,3,5-triazin-2-yl)(4-(meth ylsulfonyl)piperazin-1-yl)methanone

4-(4-(methylsulfonyl)piperazine-1-carbonyl)-6-phenyl-1,3,5-triazine-2-carbaldehyde (76 mg, 0.201 mmol), (1R,2S)-2-(4-fluorophenyl)cyclopropane-1-amine (36 mg, 0.201 mmol), acetic acid (71 uL), methanol (400 uL) and DCE (5 mL) are added to a reaction flask sequentially, subjected to nitrogen displacement for three times, and reacted for 1 h at room temperature while stirring. When the reaction is completed, the reaction solution is then added sodium cyanoborohydride (64 mg, 1.005 mmol), stirred for 1 h at room temperature till the reaction is completed, quenched with sodium bicarbonate aqueous solution (20 mL), and extracted with ethyl acetate (20 mL×2); organic phase is combined, washed with saturated saline (20 mL), dried with anhydrous sodium sulfate, and filtered; and organic phase is concentrated under a reduced pressure; and the residue is purified by Prep-HPLC (separation method 4) to obtain (4-(((1R,2S)-2-(4-fluorophenyl)cyclopropyl)amino)methyl)-6-phenyl-1,3,5-triazin-2-yl)(4-(meth ylsulfonyl)piperazin-1-yl)methanone with a yield of 10.5%.

¹H NMR (400 MHz, DMSO-d₆) δ ppm 8.46 (d, J=7.8 Hz, 2H), 7.70 (t, J=7.4 Hz, 1H), 7.60 (t, J=7.6 Hz, 2H), 7.09-6.90 (m, 4H), 4.07 (s, 2H), 3.78 (s, 2H), 3.48 (d, J=26.8 Hz, 2H), 3.10 (d, J=5.2 Hz, 2H), 2.93 (s, 3H), 1.93 (s, 1H), 1.24 (s, 2H), 1.12-1.04 (m, 1H), 0.94 (t, J=6.2 Hz, 1H), 0.85 (d, J=7.4 Hz, 1H).

ESI-MS(m/z)=511.2 [M+H]⁺.

### Example 251

### Preparation of (2-(4-(1H-pyrazol-1-yl)phenoxy)-6-(((1R,2S)-2-(4-fluorophenyl)cyclopropyl)amino)methyl)pyri midin-4-yl)(4-(methylsulfonyl)piperazin-1-yl)methanone

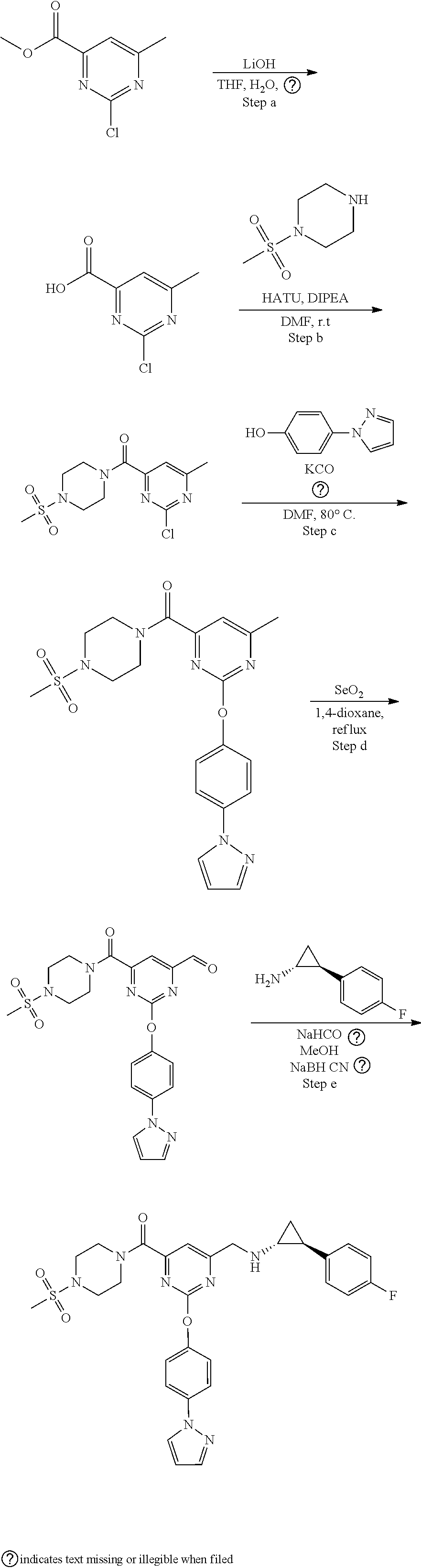

### Step a): Preparation of 2-chloro-6-methylpyrimidine-4-carboxylic acid

Methyl 2-chloro-6-methylpyrimidine-4-carboxylate (200 mg, 1.075 mmol), lithium hydroxide monohydrate (135 mg, 3.225 mmol), tetrahydrofuran (2 mL) and water (2 mL) are added to a reaction flask and reacted for 2 h at room temperature while stirring. The reaction solution is concentrated under a reduced pressure to remove organic phase; the residue is slowly added dropwise with concentrated hydrochloric acid (1 mL) and water (10 mL) under ice bath agitation, and extracted with ethyl acetate (10 mL×3); organic phase is combined, and washed with saturated saline (10 mL×2); the organic phase is concentrated under a reduced pressure to dryness; and the residue is purified by silica gel chromatography (eluent: dichloromethane/methanol=20/1) to obtain 2-chloro-6-methylpyrimidine-4-carboxylic acid with a yield of 90.2.

ESI-MS(m/z)=173.0[M+H]⁺.

### Step b): Preparation of (2-chloro-6-methylpyrimidin-4-yl) (4-(methylsulfonyl)piperazin-1-yl)methanone

2-chloro-6-methylpyrimidine-4-carboxylic acid (167 mg, 0.968 mmol), 1-(methylsulfonyl)piperazine (159 g, 0.968 mmol) and DMF (5 mL) are added to a reaction flask, added with HATU (191 mg, 1.162 mmol) and DIPEA (375 mg, 2.904 mmol) under ice bath agitation, and reacted at room temperature for 1 h. When the reaction is completed, the reaction solution is quenched with water (20 mL), and extracted with ethyl acetate (20 mL×2); organic phase is combined, sequentially washed with a saturated sodium bicarbonate aqueous solution (20 mL) and saturated saline (20 mL×2), dried with anhydrous sodium sulfate, and filtered to obtain filtrate; the filtrate is concentrated under a reduced pressure; and the residue is purified by silica gel chromatography (eluent: dichloromethane/methanol=10/1) to obtain (2-chloro-6-methylpyrimidin-4-yl) (4-(methylsulfonyl)piperazin-1-yl)methanone with a yield of 64.1%.

¹H NMR (400 MHz, Chloroform-d) δ ppm 7.39 (s, 1H), 3.86-3.79 (m, 2H), 3.64 (q, J=9.2, 7.0 Hz, 2H), 3.29 (dt, J=7.2, 5.0 Hz, 4H), 2.77 (s, 3H), 2.54 (s, 3H).

ESI-MS(m/z)=319.1 [M+H]+.

### Step c): Preparation of (2-(4-(1H-pyrazol-1-yl)phenoxy)-6-methylpyrimidin-4-yl)(4-(methyl sulfonyl)piperazin-1-yl)met hanone

(2-chloro-6-methylpyrimidin-4-yl)(4-(methylsulfonyl)piperazin-1-yl)methanone (197 mg, 0.620 mmol), 4-(1H-pyrazol-1-yl)phenol (149 mg, 0.930 mmol), potassium carbonate (257 mg, 1.860 mmol) and DMF (5 mL) are added to a reaction flask sequentially and stirred at 80° C. for 12 h. When the reaction is completed, the reaction solution is quenched with water (20 mL), and extracted with ethyl acetate (20 mL×3); organic phase is combined, washed with saturated saline (20 mL×2), and concentrated under a reduced pressure to dryness; and the residue is purified by silica gel chromatography (eluent: dichloromethane/methanol=10/1) to obtain (2-(4-(1H-pyrazol-1-yl)phenoxy)-6-methylpyrimidin-4-yl)(4-(methylsulfonyl)piperazin-1-yl)met hanone with a yield of 50.0%.

ESI-MS(m/z)=443.1[M+H]⁺.

### Step d): Preparation of 2-(4-(1H-pyrazol-1-yl)phenoxy)-6-(4-(methylsulfonyl)piperazin-1-carbonyl)pyrimidine-4-forma mide

(2-(4-(1H-pyrazol-1-yl)phenoxy)-6-methylpyrimidin-4-yl)(4-(methylsulfonyl)piperazin-1-yl)met hanone (206 mg, 0.465 mmol), tin dioxide (258 mg, 2.325 mmol) and 1,4-dioxane (10 mL) are added to a reaction flask sequentially, and reacted at 100° C. for 12 h while stirring. When the reaction is completed, the reaction solution is concentrated under a reduced pressure; and the residue is purified by silica gel chromatography (eluent: dichloromethane/methanol=10/1) to obtain 2-(4-(1H-pyrazol-1-yl)phenoxy)-6-(4-(methylsulfonyl)piperazin-1-carbonyl)pyrimidine-4-forma mide with a yield of 62.0%.

ESI-MS(m/z)=457.1[M+H]⁺.

### Step e): Preparation of (2-(4-(1H-pyrazol-1-yl)phenoxy)-6-(((1R,2S)-2-(4-fluorophenyl)cyclopropyl)amino)methyl)pyri midin-4-yl)(4-(methylsulfonyl)piperazin-1-yl)methanone

2-(4-(1H-pyrazol-1-yl)phenoxy)-6-(4-(methylsulfonyl)piperazin-1-carbonyl)pyrimidine-4-forma mide (132 mg, 0.288 mmol), (1R,2S)-2-(4-fluorophenyl)cyclopropane-1-amine (54 mg, 0.288 mmol), acetic acid (132 uL), methanol (660 uL), and DCE (10 mL) are added to a reaction flask sequentially, subjected to nitrogen displacement for three times, and reacted for 1 h at room temperature while stirring. When the reaction is completed, the reaction solution is then added with sodium cyanoborohydride (120 mg, 1.440 mmol), stirred for 1 h at room temperature till the reaction is ended, quenched with a sodium bicarbonate aqueous solution (20 mL), and extracted with ethyl acetate (20 mL×2); organic phase is combined, washed with saturated saline (20 mL), dried with anhydrous sodium sulfate, and filtered; the organic phase is concentrated under a reduced pressure; and the residue is purified by Prep-HPLC (separation method 4) to obtain (2-(4-(1H-pyrazol-1-yl)phenoxy)-6-(((1R,2S)-2-(4-fluorophenyl)cyclopropyl)amino)methyl)pyri midin-4-yl)(4-(methylsulfonyl)piperazin-1-yl)methanone with a yield of 55.5%.

¹H NMR (400 MHz, DMSO-d₆) δppm 8.48 (d, J=2.4 Hz, 1H), 8.00-7.81 (m, 2H), 7.75 (d, J=1.8 Hz, 1H), 7.49 (s, 1H), 7.42-7.29 (m, 2H), 7.21-6.87 (m, 4H), 6.55 (t, J=2.2 Hz, 1H), 3.96 (s, 1H), 3.67 (t, J=5.2 Hz, 2H), 3.46 (t, J=4.8 Hz, 2H), 3.13 (t, J=5.4 Hz, 2H), 2.91 (t, J=5.2 Hz, 2H), 2.72 (s, 3H), 1.92 (s, 3H), 1.02 (d, J=39.4 Hz, 2H).

ESI-MS(m/z)=592.2 [M+H]⁺.

### Example 252

### Preparation of 4-(6-(4-aminopiperidin-1-yl)-3-((2-(3-fluoro-4-methoxyphenyl)cyclopropyl)amino)methyl)-4-m ethoxypyridin-2-yl)-2-fluorobenzonitrile

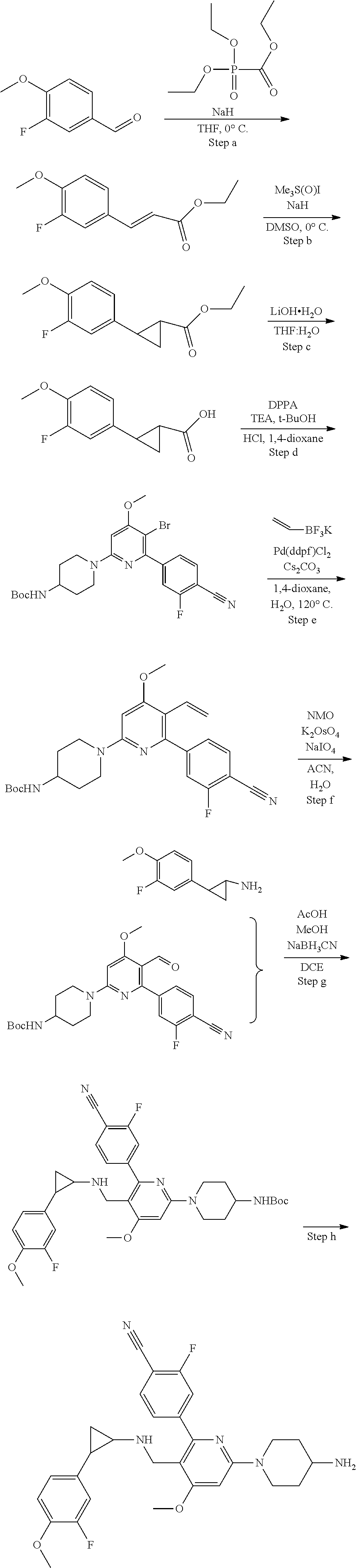

### Step a): Preparation of ethyl (E)-3-(3-fluoro-4-methoxyphenyl)acrylate

Ethyl(diethoxyphosphoryl)carboxylate (4.1 g, 19.481 mmol) and THE (20 mL) are added to a reaction flask, added with NaH (779 mg, 19.481 mmol, 60%) under ice bath agitation, and reacted at room temperature for 30 min while stirring. 3-fluoro-4-methoxybenzaldehyde (2 g, 12.987 mmol) is added under ice bath agitation, and reacted for 30 min at room temperature while stirring. When the reaction is completed, the reaction solution is quenched with water (100 mL), and extracted with ethyl acetate (100 mL×3); organic phase is combined, and washed with saturated saline (100 mL×2); the organic phase is concentrated under a reduced pressure to dryness; and the residue is purified by silica gel chromatography (eluent: petroleum ether/ethyl acetate=1/1) to obtain ethyl (E)-3-(3-fluoro-4-methoxyphenyl)acrylate with a yield of 80.5%.

ESI-MS(m/z)=225.1[M+H]⁻

### Step b): Preparation of ethyl (2-(3-fluoro-4-methoxyphenyl)cyclopropane-1-carboxylate

Trimethylsulfoxide iodide (3.1 g, 15.585 mmol) and DMSO (20 mL) are added to a reaction flask, added with NaH (623 mg, 15.585 mmol, 60%) under ice bath agitation, and reacted at room temperature for 30 min while stirring. Ethyl (E)-3-(3-fluoro-4-methoxyphenyl)acrylate (2.3 g, 10.390 mmol) is added under ice bath agitation, and reacted for 30 min at room temperature while stirring. When the reaction is completed, the reaction solution is quenched with water (100 mL), and extracted with ethyl acetate (100 mL×3); organic phase is combined, washed with saturated saline (100 mL×2), and concentrated under a reduced pressure to dryness; and the residue is purified by silica gel chromatography (eluent: petroleum ether/ethyl acetate=1/1) to obtain ethyl (2-(3-fluoro-4-methoxyphenyl)cyclopropane-1-carboxylate with a yield of 42.5%.

¹H NMR (400 MHz, Chloroform-d) δppm 6.92-6.77 (m, 3H), 4.17 (q, J=7.2 Hz, 2H), 3.86 (s, 3H), 2.46 (ddd, J=9.2, 6.4, 4.2 Hz, 1H), 1.82 (ddd, J=8.4, 5.4, 4.2 Hz, 1H), 1.56 (ddd, J=9.2, 5.4, 4.6 Hz, 2H), 1.32-1.18 (m, 3H).

ESI-MS(m/z)=239.1[M+H]⁺.

### Step c): Preparation of 2-(3-fluoro-4-methoxyphenyl)cyclopropane-1-carboxylic acid

Ethyl 2-(3-fluoro-4-methoxyphenyl)cyclopropane-1-carboxylate (1.1 g, 4.416 mmol), lithium hydroxide monohydrate (556 mg, 13.248 mmol), tetrahydrofuran (10 mL) and water (10 mL) are added to a reaction flask and reacted at 40° C. for 2 h while stirring. The reaction solution is concentrated under a reduced pressure to remove organic phase, slowly added dropwise with concentrated hydrochloric acid (3 mL) and water (20 mL) under ice bath agitation, and extracted with ethyl acetate (40 mL×3); organic phase is combined, and washed with saturated saline (40 mL×2); the organic phase is concentrated under a reduced pressure to dryness; and the residue is purified by silica gel chromatography (eluent: petroleum ether/ethyl acetate=1/1) to obtain 2-(3-fluoro-4-methoxyphenyl)cyclopropane-1-carboxylic acid with a yield of 85.0%.

¹H NMR (400 MHz, Chloroform-d) δ 6.99-6.62 (m, 3H), 3.87 (s, 3H), 2.54 (ddd, J=9.2, 6.6, 4.2 Hz, 1H), 1.83 (ddd, J=8.4, 5.2, 4.2 Hz, 1H), 1.63 (dt, J=9.8, 4.8 Hz, 1H), 1.34 (ddd, J=8.4, 6.8, 4.8 Hz, 1H).

ESI-MS(m/z)=210.0[M+H]⁺.

### Step d): Preparation of 2-(3-fluoro-4-methoxyphenyl)cyclopropane-1-amine

2-(3-fluoro-4-methoxyphenyl)cyclopropane-1-carboxylic acid (200 mg, 0.952 mmol), DPPA (393 mg, 1.428 mmol), TEA (144 mg, 1.428 mmol), and toluene (5 mL) are added to a reaction flask and reacted at 100° C. for 30 min while stirring. Concentrated hydrochloric acid (1 mL) is then added dropwise to the reaction solution in a reflux state. When the reaction is completed, the reaction solution is poured into a sodium bicarbonate aqueous solution (40 mL) in an ice bath, and extracted with ethyl acetate (40 mL×3); organic phase is combined, washed with saturated saline (40 mL×2), and concentrated under a reduced pressure to dryness; and the residue is purified by silica gel chromatography (eluent: petroleum ether/ethyl acetate=1/2) to obtain 2-(3-fluoro-4-methoxyphenyl)cyclopropane-1-amine with a yield of 91.5%.

ESI-MS(m/z)=182.2[M+H]⁺.

### Step e): Preparation of tert-butyl(1-(6-(4-cyano-3-fluorophenyl)-4-methoxy-5-vinylpyridin-2-yl)piperidin-4-yl)carbamat e

### Tert-butyl

(1-(5-bromo-6-(4-cyano-3-fluorophenyl)-4-methoxypyridin-2-yl)piperidin-4-yl)carbamate (110 mg, 0.218 mmol), vinyl potassium trifluoroborate (45 mg, 0.327 mmol), Cs₂CO₃ (142 mg, 0.436 mmol), Pd(dppf)Cl₂ (16 mg, 0.022 mmol), 1.4-Dioxane (4 mL) and H₂O (1 mL) are added to a reaction flask, subjected to nitrogen displacement for three times, and reacted at 120° C. for 1 h while stirring. The reaction solution is concentrated under a reduced pressure to dryness; and the residue is purified by silica gel chromatography (eluent: petroleum ether/ethyl acetate=1/1) to obtain tert-butyl(1-(6-(4-cyano-3-fluorophenyl)-4-methoxy-5-vinylpyridin-2-yl)piperidin-4-yl)carbamat e with a yield of 85.0%.

ESI-MS(m/z)=453.1[M+H]⁺.

### Step f): Preparation of tert-butyl (1-(6-(4-cyano-3-fluorophenyl)-5-formyl-4-methoxypyridin-2-yl)piperidin-4-yl)carbamate

### Tert-butyl

(1-(6-(4-cyano-3-fluorophenyl)-4-methoxy-5-vinylpyridin-2-yl)piperidin-4-yl)carbamate (84 mg, 0.185 mmol), NMO (133 uL, 0.555 mmol, 50%), potassium osmate (14 mg, 0.019 mmol), THE (6 ml) and water (2 mL) are added to a reaction flask and reacted for 1 h while stirring. When the reaction is completed, the reaction solution is added with sodium periodate (244 mg, 0.555 mmol), stirred for 1 h at room temperature till the reaction is completed, then added with a sodium sulfite aqueous solution (20 mL), and extracted with ethyl acetate (20 mL×3); organic phase is combined, washed with saturated saline (20 mL×2), dried with anhydrous sodium sulfate, and filtered to obtain filtrate; the filtrate is concentrated under a reduced pressure; and the residue is purified by silica gel chromatography (eluent: petroleum ether/ethyl acetate=1/1) to obtain tert-butyl (1-(6-(4-cyano-3-fluorophenyl)-5-formyl-4-methoxypyridin-2-yl)piperidin-4-yl)carbamate with a yield of 75.0%.

ESI-MS(m/z)=455.3[M+H]⁺.

### Step g): Preparation of tert-butyl(1-(6-(4-cyano-3-fluorophenyl)-5-(2-(3-fluoro-4-methoxyphenyl)cyclopropyl)amino)m ethyl)-4-methoxypyridin-2-yl)piperidin-4-yl)carbamate

### Tert-butyl

(1-(6-(4-cyano-3-fluorophenyl)-5-formyl-4-methoxypyridin-2-yl)piperidin-4-yl)carbamate (63 mg, 0.139 mmol), 2-(3-fluoro-4-methoxyphenyl)cyclopropane-1-amine (25 mg, 0.139 mmol), acetic acid (63 uL), methanol (315 uL), and DCE (5 mL) are added to a reaction flask sequentially, subjected to nitrogen displacement for three times, and reacted for 1 h at room temperature while stirring. When the reaction is completed, the reaction solution is then added with sodium cyanoborohydride (56 mg, 0.695 mmol), stirred for 1 h at room temperature till the reaction is completed, quenched with a sodium bicarbonate aqueous solution (20 mL), and extracted with ethyl acetate (20 mL×2); organic phase is combined, washed with saturated saline (20 mL), dried with anhydrous sodium sulfate, filtered, and concentrated under a reduced pressure; and the residue is purified by Prep-HPLC (separation method 4) to obtain tert-butyl(1-(6-(4-cyano-3-fluorophenyl)-5-(2-(3-fluoro-4-methoxyphenyl)cyclopropyl)amino)m ethyl)-4-methoxypyridin-2-yl)piperidin-4-yl)carbamate with a yield of 73.0%.

ESI-MS m/z)=620.3 [M+H]⁺.

### Step h): Preparation of 4-(6-(4-aminopiperidin-1-yl)-3-((2-(3-fluoro-4-methoxyphenyl)cyclopropyl)amino)methyl)-4-m ethoxypyridin-2-yl)-2-fluorobenzonitrile

Tert-butyl(1-(6-(4-cyano-3-fluorophenyl)-5-(2-(3-fluoro-4-methoxyphenyl)cyclopropyl)amino)m ethyl)-4-methoxypyridin-2-yl)piperidin-4-yl)carbamate (63 mg, 0.101 mmol) is added to a reaction flask, then added with 4M hydrogen chloride (ethyl acetate solution, 2.5 mL) and stirred at room temperature for 1 h; a large amount of solids precipitate, and then concentrated under a reduced pressure to obtain a crude product; and the crude product is purified by Prep-HPLC (separation method 4) to obtain 4-(6-(4-aminopiperidin-1-yl)-3-((2-(3-fluoro-4-methoxyphenyl)cyclopropyl)amino)methyl)-4-m ethoxypyridin-2-yl)-2-fluorobenzonitrile with a yield of 63.0%.

¹H NMR (400 MHz, DMSO-d₆) δppm 7.94 (dd, J=8.0, 7.0 Hz, 1H), 7.78 (dd, J=10.8, 1.6 Hz, 1H), 7.67 (dd, J=8.0, 1.4 Hz, 1H), 6.99 (t, J=8.8 Hz, 1H), 6.81-6.64 (m, 2H), 6.41 (d, J=8.4 Hz, 1H), 4.25 (t, J=15.0 Hz, 2H), 3.81 (d, J=10.4 Hz, 6H), 3.64-3.50 (m, 2H), 3.01-2.84 (m, 2H), 2.77 (dq, J=9.8, 4.8, 3.8 Hz, 1H), 2.13 (dt, J=7.4, 3.8 Hz, 1H), 1.75 (d, J=12.8 Hz, 2H), 1.68 (ddd, J=9.0, 5.6, 2.8 Hz, 1H), 1.21 (s, 2H), 0.93 (dt, J=9.4, 4.8 Hz, 1H), 0.89-0.80 (m, 1H).

ESI-MS m/z)=520.2 [M+H]⁺.

### Example 253

### Preparation of 6-(((1R,2S)-2-(4-fluorophenyl)cyclopropyl)amino)methyl)—N-(tetrahydro-2H-pyran-4-yl)pyrimid in-4-carboxamide hydrochloride

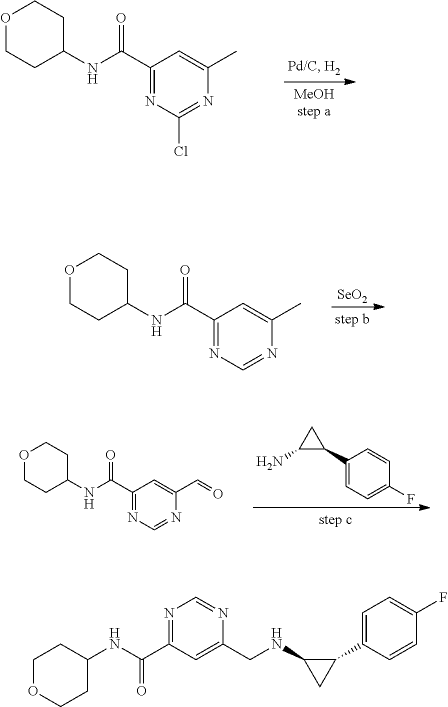

### Step a): Synthesis of 6-methyl-N-(tetrahydro-2H-pyran-4-yl)pyrimidine-4-carboxamide

2-chloro-6-methyl-N-(tetrahydro-2H-pyran-4-yl)pyrimidine-4-carboxamide (550 mg, 2.16 mmol), 10% palladium carbon (55 mg), sodium acetate (55 mg, 0.67 mmol), and anhydrous methanol (5 mL) are added to a reaction flask sequentially, subjected to hydrogen displacement for three times, and reacted for 3 h; when LC-MS monitors that the reaction is completed, the reaction solution is filtered with diatomite, and concentrated to dryness; and the residue is purified by silica gel chromatography (eluent: ethyl acetate/petroleum ether=1/1) to obtain 6-methyl-N-(tetrahydro-2H-pyran-4-yl)pyrimidin-4-carboxamide with a yield of 55.2%.

¹H NMR (400 MHz, DMSO-d₆) δ ppm 9.15 (d, J=1.4 Hz, 1H), 8.83 (d, J=8.4 Hz, 1H), 7.91 (s, 1H), 4.09-3.96 (m, 1H), 3.87 (dt, J=11.4, 3.4 Hz, 2H), 3.38 (ddd, J=11.6, 7.8, 5.4 Hz, 2H), 2.57 (s, 3H), 1.70 (h, J=4.0 Hz, 4H).

ESI-MS m/z: 222.1[M+H]⁺.

### Step b): Synthesis of 6-formyl-n-(tetrahydropyran-4-yl)pyrimidin-4-carboxamide

6-methyl-N-(tetrahydro-2H-pyran-4-yl)pyrimidin-4-carboxamide (170 mg, 0.77 mmol), selenium dioxide (427 mg, 3.85 mmol) and 1,4-dioxane (10 mL) are added to a reaction flask, warmed to 110° C. under nitrogen protection, and reacted for 16 h while stirring. When LC-MS shows that the reaction is completed, the reaction solution is added with a saturated sodium bicarbonate aqueous solution (15 mL), extracted with ethyl acetate (15 mL×3), washed with saturated saline (10 mL), dried with anhydrous sodium sulfate, filtered, and concentrated to obtain 6-formyl-n-(tetrahydropyran-4-yl)pyrimidin-4-carboxamide with a yield of 46.4%.

ESI-MS m/z: 236.1[M+H]⁺.

### Step c): Synthesis of 6-(((1R,2S)-2-(4-fluorophenyl)cyclopropyl)amino)methyl)—N-(tetrahydro-2H-pyran-4-yl)pyrimid ine-4-carboxamide

6-formyl-n-(tetrahydropyran-4-yl)pyrimidin-4-carboxamide (84 mg, 0.36 mmol) is dissolved in 1,2-dichloroethane (2 mL), then added with acetic acid (214 mg, 3.57 mmol), methanol (574 mg, 17.87 mmol), and (1R,2S)-2-(4-fluorophenyl)cyclopropane-1-amine (67.0 mg, 0.36 mmol), reacted for 1.5 h while stirring under nitrogen protection, added with sodium cyanoborohydride (90 mg, 1.43 mmol), and continues to react for 16 h. When LC-MS shows that the reaction is completed, the reaction solution is quenched with water (3 mL), then extracted with ethyl acetate (10 mL×3), washed with saturated saline (10 mL), dried with anhydrous sodium sulfate, filtered and concentrated, purified by pre-HPLC preparation (separation method 1), and freeze-dried to obtain 6-(((1R,2S)-2-(4-fluorophenyl)cyclopropyl)amino)methyl)—N-(tetrahydro-2H-pyran-4-yl)pyrimid in-4-carboxamide with a yield of 3.2%.

¹H NMR (400 MHz, Methanol-d₄) δ ppm 8.52 (s, 1H), 7.34 (s, 1H), 6.42-6.35 (m, 2H), 6.27-6.19 (m, 2H), 3.93 (s, 2H), 3.33 (dddt, J=12.6, 8.2, 6.8, 4.4 Hz, 1H), 3.19 (ddd, J=12.0, 4.2, 2.2 Hz, 2H), 2.73 (td, J=11.8, 2.2 Hz, 2H), 2.29 (ddd, J=8.0, 4.4, 3.6 Hz, 1H), 1.74 (ddd, J=10.4, 6.6, 3.6 Hz, 1H), 1.08 (ddd, J=12.6, 4.6, 2.2 Hz, 2H), 0.99-0.88 (m, 2H), 0.78 (ddd, J=10.4, 6.8, 4.4 Hz, 1H), 0.60 (dt, J=7.8, 6.8 Hz, 1H).

ESI-MS m/z: 371.2[M+H]⁺.

### Example 254

### Preparation of 3-(2-((1R,2S)-2-(4-fluorophenyl)cyclopropyl)aminoethoxy)-5-(4-(methylsulfonyl)piperazin-1-yl)-[1,1-biphenyl]-4-nitrile

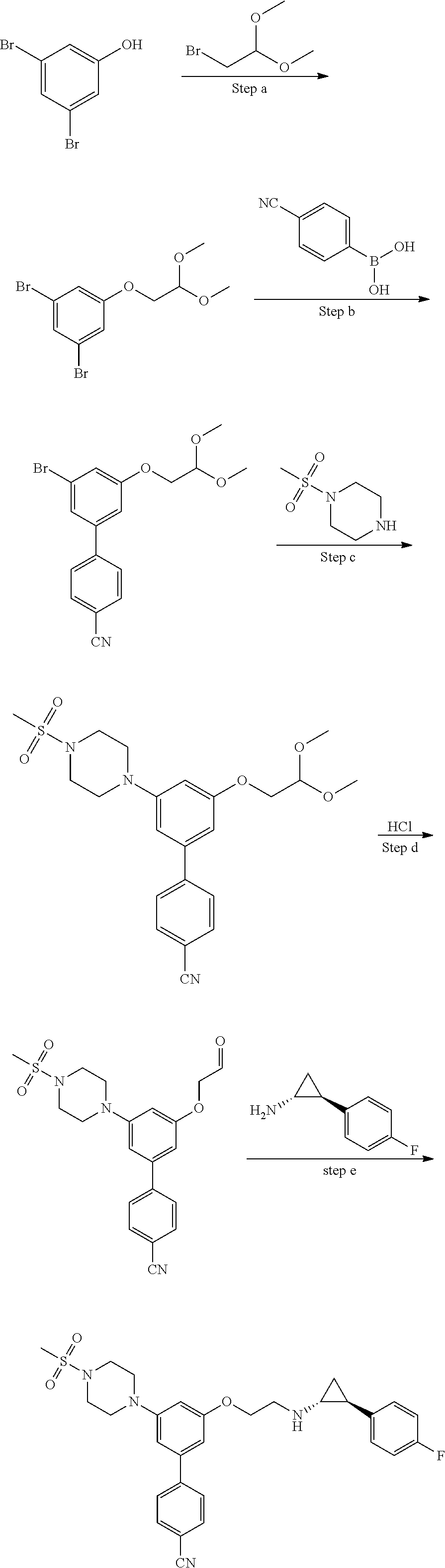

### Step a): Synthesis of 1,3-dibromo-5-(2,2-dimethoxy)benzene

A compound 3,5-dibromophenol (2.00 g, 8.01 mmol), 2-bromo-1,1-dimethoxyethane (2.03 g, 12.01 mmol), cesium carbonate (7.82 g, 24.03 mmol) and 40 mL of dry DMF are added sequentially to a reaction flask, and reacted at 800° C. overnight in an oil bath under nitrogen protection; when LC-MS shows that the reaction is completed, the reaction solution is diluted with water (40 mL), and extracted with ethyl acetate (30 mL×3); organic phase is combined. washed with saturated saline, dried with anhydrous sodium sulfate, filtered, and concentrated under a reduced pressure; and the residue is purified by silica gel chromatography (eluent: ethyl acetate/petroleum ether=1/10) to obtain 1,3-dibromo-5-(2,2-dimethoxy)benzene with a yield of 93.6%.

¹H NMR (400 MHz, Methanol-d₄) δ ppm 7.28 (t, J=1.6 Hz, 1H), 7.11 (d, J=1.6 Hz, 2H), 4.68 (t, J=5.2 Hz, 1H), 3.99 (d, J=5.2 Hz, 2H), 3.43 (s, 6H).

### Step b): Synthesis of 3′-bromo-5′-(2,2-dimethoxyethoxy)-[1,1′-biphenyl]-4-carbonitrile

1,3-dibromo-5-(2,2-dimethoxy)benzene (1.35 g, 3.99 mmol), p-cyanophenylboronic acid (587 mg, 3.99 mmol), Pd(dppf)Cl₂ (584 mg, 0.79 mmol), Na₂CO₃ (847 mg, 7.98 mmol), 15 mL of 1,4-dioxane and 3 mL of water are added to a reaction flask, purged by nitrogen blowing, and reacted at 80° C. in a microwave reactor for 30 min; a reaction progress is monitored by TLC (petroleum ether/ethyl acetate=3/1) and LC-MS. When LC-MS shows that the reaction is completed, the reaction solution is suction-filtered by a sand core funnel to obtain filtrate; the filtrate is concentrated to dryness; and the residue is purified by silica gel chromatography (eluent: ethyl acetate/petroleum ether=1/6) to obtain 3′-bromo-5′-(2,2-dimethoxyethoxy)-[1,1′-biphenyl]-4-carbonitrile with a yield of 68.44%.

¹H NMR (400 MHz, Chloroform-d) δ ppm 7.75-7.70 (m, 2H), 7.65-7.61 (m, 2H), 7.32 (t, J=1.6 Hz, 1H), 7.13 (t, J=2.0 Hz, 1H), 7.07 (t, J=1.8 Hz, 1H), 4.73 (t, J=5.2 Hz, 1H), 4.04 (d, J=5.2 Hz, 2H), 3.47 (s, 6H).

### Step c): Synthesis of 3′-(2,2-dimethoxyethoxy)-5′-(4-(methylsulfonyl)piperazin-1-yl)-[1,1′-biphenyl]-4-carbon

3′-bromo-5′-(2,2-dimethoxyethoxy)-[1,1′-biphenyl]-4-carbonitrile (300 mg, 0.83 mmol), 1-(methylsulfonyl)piperazine (204 mg, 1.25 mmol), PD₂(dba)₃ (152 mg, 0.16 mmol), RuPhos (155 mg, 0.33 mmol), Cs₂CO₃ (811 mg, 2.49 mmol) and 13 mL of toluene are sequentially added to a reaction bottle, subjected to nitrogen displacement for three times, warmed to 90° C. and reacted overnight. The reaction progress is monitored by TLC (petroleum ether/ethyl acetate=3/1) and LC-MS; when the reaction is completed, the reaction solution is cooled, and suction-filtered with a sand core funnel to obtain filtrate; the filtrate is concentrated to dryness; and the residue is purified by silica gel chromatography (eluent: ethyl acetate/petroleum ether=2/1) to obtain 3′-(2,2-dimethoxyethoxy)-5′-(4-(methylsulfonyl)piperazin-1-yl)-[1,1′-biphenyl]-4-carbon with a yield of 75.9%.

¹HNMR (400 MHz, DMSO-d₆) δ ppm 7.87 (s, 4H), 6.87 (s, 1H), 6.73 (s, 1H), 6.58 (d, J=2.8 Hz, 1H), 4.67 (t, J=5.2 Hz, 1H), 4.03 (d, J=5.2 Hz, 2H), 3.59 (d, J=2.2 Hz, 6H), 3.33 (d, J=5.2 Hz, 8H), 2.90 (s, 3H).

ESI-MS m/z: 446.2[M+H]⁺.

### Step d): Synthesis of 3′-(4-(methylsulfonyl)piperazin-1-yl)-5′-(2-oxyethoxy)-[1,1′-biphenyl]-4-carbonitrile

3′-(2,2-dimethoxyethoxy)-5′-(4-(methylsulfonyl)piperazin-1-yl)-[1,1′-biphenyl]-4-carbon (120 mg, 0.27 mmol) is dissolved in a 4M hydrochloric acid (dioxane solution, 8 mL) and stirred for 2 h at room temperature under nitrogen protection. When TLC and LC-MS monitor that the reaction is completed, the reaction solution is extracted with ethyl acetate (10 mL×3), washed with saturated saline (10 mL), dried with anhydrous sodium sulfate, filtered, and concentrated to obtain 3′-(4-(methylsulfonyl)piperazin-1-yl)-5′-(2-oxyethoxy)-[1,1′-biphenyl]-4-carbonitrile to obtain a crude product; and the crude product is directly used in the next step.

ESI-MS m/z: 400.1[M+H]⁺.

### Step e): Synthesis of 3′-(2-((1R,2S)-2-(4-fluorophenyl)cyclopropyl)aminoethoxy)-5′-(4-(methylsulfonyl)piperazin-1-y 1)-[1,1′-biphenyl]-4-nitrile

3′-(4-(Methylsulfonyl)piperazin-1-yl)-5′-(2-oxyethoxy)-[1,1′-biphenyl]-4-carbonitrile (134 mg, 0.34 mmol) and (1R,2S)-2-(4-fluorophenyl)cyclopropylamine hydrochloride (50.6 mg, 0.34 mmol) are weighed and dissolved in dry 1,2-dichloroethane (5 mL), added with acetic acid (33 ul) and anhydrous methanol (166 ul), and reacted at room temperature for 2 h under nitrogen protection; when LC-MS shows that the raw materials disappear, the reaction solution is added with sodium triacetoxyborohydride (142 mg, 0.67 mmol) and continues to react for 2 h; and LC-MS shows that the reaction is completed. The reaction solution is quenched with a saturated ammonium chloride aqueous solution (3 mL), extracted with dichloromethane (10 mL×3), washed with saturated saline (6 mL), and dried with anhydrous sodium sulfate, and no residue exists in aqueous LC-MS. The reaction solution is then concentrated under a reduced pressure to dryness, purified directly by a silica gel preparation chromatographic plate (methanol/dichloromethane=1/12), concentrated, and freeze-dried to obtain 3′-(2-((1R,2S)-2-(4-fluorophenyl)cyclopropyl)aminoethoxy)-5′-(4-(methylsulfonyl)piperazin-1-y 1)-[1,1′-biphenyl]-4-nitrile with a yield of 11.7%.

¹HNMR (400 MHz, DMSO-d₆) δ ppm 7.85 (q, J=8.2 Hz, 4H), 7.03 (p, J=8.6 Hz, 4H), 6.84 (s, 1H), 6.68 (s, 1H), 6.52 (s, 1H), 4.08 (t, J=5.8 Hz, 2H), 3.30 (d, J=5.0 Hz, 4H), 3.23 (d, J=4.6 Hz, 4H), 2.94 (d, J=5.8 Hz, 2H), 2.89 (s, 3H), 2.28 (dd, J=7.2, 3.8 Hz, 1H), 1.82 (d, J=8.4 Hz, 1H), 1.03-0.86 (m, 2H).

ESI-MS m/z: 535.2[M+H]⁺.

### Example 255

### Preparation of (2-(4-(1H-pyrazol-1-yl)phenyl)-6-(2-(((1R,2S)-2-(4-fluorophenyl)cyclopropyl)amino)ethyl)pyrid in-4-yl) (4-(methylsulfonyl)piperazin-1-yl)methanone

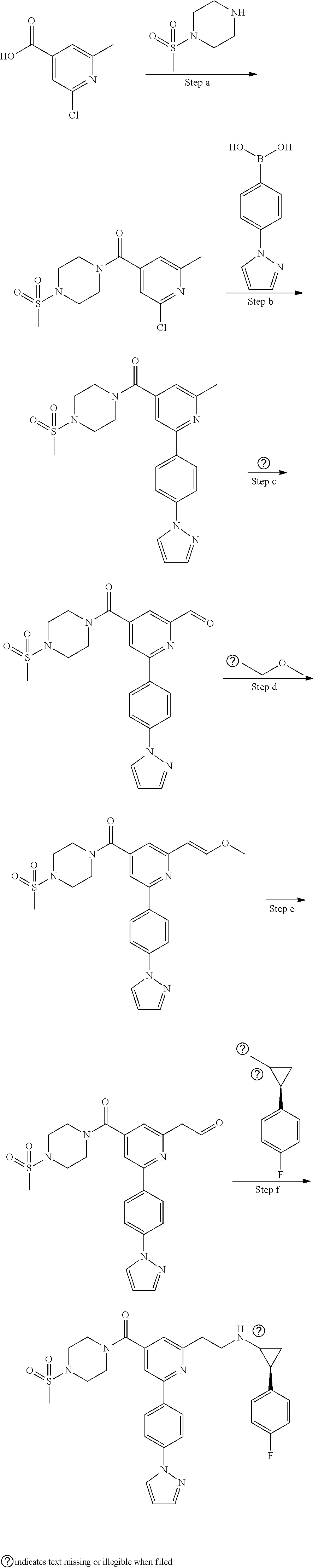

### Step a): Preparation of (2-chloro-6-methylpyridin-4-yl)(4-(methylsulfonyl)piperazin-1-yl) methanone

2-chloro-6-methylisonicotinic acid (2 g, 11.70 mmol), 1-(methylsulfonyl)piperazine (133.6 mg, 0.26 mmol), Pd(dppf)Cl₂ (2.3 g, 14.02 mmol), HATU (5.3 g, 13.95 mmol), DIEA (4.5 g, 34.88 mmol) and N,N-dimethylformamide (30 mL) are sequentially added to a 50 ml reaction flask and reacted for 1 h at room temperature while stirring. When the reaction is completed, the reaction solution is quenched with water (50 mL), and extracted with ethyl acetate (50 mL×2); organic phase is combined, washed with saturated saline (50 mL×2), dried with anhydroous sodium sulfate, and filtered to obtain filtrate; the filtrate is concentrated under a reduced pressure; and the residue is purified by silica gel chromatography (eluent: petroleum ether/ethyl acetate=2/1) to obtain (2-chloro-6-methylpyridin-4-yl)(4-(methylsulfonyl)piperazin-1-yl)methanone with a yield of 97.2%.

¹H NMR (400 MHz, DMSO-d₆) δ ppm 7.38 (t, J=1.0 Hz, 1H), 7.31 (d, J=1.2 Hz, 1H), 3.71 (t, J=5.0 Hz, 2H), 3.36 (t, J=5.0 Hz, 2H), 3.21 (t, J=5.2 Hz, 2H), 3.15-3.10 (m, 2H), 2.91 (s, 3H), 2.69 (s, 3H).

ESI-MS(m/z)=318.1 [M+H]⁺.

### Step b): Preparation of (2-(4-(1H-pyrazol-1-yl)phenyl)-6-methamphetamine-4-yl) (4-(methylsulfonamide)piperazin-1-yl)methanone

(2-chloro-6-methylpyridin-4-yl)(4-(methylsulfonyl)piperazin-1-yl)methanone (4 g, 3.79 mmol), (4-(1H-pyrazol-1-yl)phenyl)boronic acid (2.85 g, 4.55 mmol), Pd(dppf)Cl₂ (924 mg, 0.38 mmol), cesium carbonate (8.23 g, 7.64 mmol), 1,4-dioxane (40 mL) and water (10 mL) are added to a reaction flask sequentially and stirred in microwaves at 120° C. for 1 h. When the reaction is completed, the reaction solution is concentrated under a reduced pressure; and the residue is purified by silica gel chromatography (eluent: petroleum ether/ethyl acetate=1/1) to obtain (2-(4-(1H-pyrazol-1-yl)phenyl)-6-methamphetamine-4-yl) (4-(methylsulfonamide)piperazin-1-yl)methanone with a yield of 76.5%.

¹H NMR (400 MHz, DMSO-d₆) δ ppm 8.63 (d, J=2.6 Hz, 1H), 8.33-8.25 (m, 3H), 8.04-8.00 (m, 2H), 7.81 (d, J=1.8 Hz, 1H), 7.70 (d, J=1.8 Hz, 1H), 6.60 (t, J=2.2 Hz, 1H), 3.82 (t, J=5.0 Hz, 2H), 3.66 (t, J=4.8 Hz, 2H), 3.29 (t, J=5.2 Hz, 2H), 3.22 (t, J=5.0 Hz, 2H), 2.96 (s, 3H), 2.51 (s, 3H).

ESI-MS(m/z)=426.0 [M+H]⁺.

### Step c): Preparation of 6-(4-(1H-pyrazol-1-yl)phenyl)-4-(4-(methylsulfonyl)piperazin-1-carbonyl)pyridylaldehyde (2-(4-(1H-pyrazol-1-yl)phenyl)-6-methamphetamine-4-yl)

(4-(methylsulfonamide)piperazin-1-yl)methanone (2.0 g, 4.71 mmol), tin dioxide (8.36 g, 65.32 mmol) and 1,4-dioxane (30 mL) are added to a reaction flask sequentially, and reacted at 110° C. for 24 h while stirring. When the reaction is completed, the reaction solution is quenched with a saturated sodium bicarbonate aqueous solution (50 mL), and extracted with ethyl acetate (50 mL×2); organic phase is combined, washed with saturated saline (50 mL×2), dried with anhydrous sodium sulfate, and filtered to obtain filtrate; and the filtrate is concentrated under a reduced pressure to obtain 6-(4-(1H-pyrazol-1-yl)phenyl)-4-(4-(methylsulfonyl)piperazin-1-carbonyl)pyridylaldehyde with a yield of 48.5.

ESI-MS(m/z)=458.0 [M+H₂O+H]⁺.

### Step d): Preparation of (E)-(2-(4-(1H-pyrazol-1-yl)phenyl)-6-(2-methoxyvinyl)pyridin-4-yl)(4-(methylsulfonyl)piperazi n-1-yl)methanone

6-(4-(1H-pyrazol-1-yl)phenyl)-4-(4-(methylsulfonyl)piperazin-1-carbonyl)pyridylaldehyde (1 g, 2.28 mmol), TDA-1 (883 mg, 2.73 mmol) and (methoxymethyl)triphenylphosphonium chloride (620 mg, 1.81 mmol) are sequentially added to a reaction flask containing dichloromethane/5M potassium carbonate aqueous solution (10 ml/2 ml), and stirred at room temperature for 2 h. When the reaction is completed, the reaction solution is quenched with a sodium bicarbonate aqueous solution (10 mL), and extracted with ethyl acetate (20 mL×2); organic phase is combined, washed with saturated saline (40 mL×2), dried with anhydrous sodium sulfate, filtered and concentrated under a reduced pressure; and the residue is purified by silica gel chromatography (eluent: petroleum ether/ethyl acetate=1/1) to obtain (E)-(2-(4-(1H-pyrazol-1-yl)phenyl)-6-(2-methoxyvinyl)pyridin-4-yl)(4-(methylsulfonyl)piperazi n-1-yl)methanone with a yield of 9.4%.

¹H NMR (400 MHz, DMSO-d₆) δ ppm 8.61 (t, J=2.2 Hz, 2H), 8.31-8.24 (m, 2H), 8.01-7.96 (m, 3H), 7.80 (t, J=1.4 Hz, 2H), 6.60 (s, 1H), 6.05 (d, J=12.6 Hz, 1H), 3.88 (s, 4H), 3.76 (s, 3H), 3.17 (d, J=5.2 Hz, 4H), 2.94 (s, 3H).

ESI-MS(m/z)=468.2[M+H]⁺.

### Step e): Preparation of 2-(6-(4-(1H-pyrazol-1-yl)phenyl)-4-(4-(methylsulfonyl)piperazin-1-carbonyl)pyridin-2-yl)acetal dehyde

(E)-(2-(4-(1H-pyrazol-1-yl)phenyl)-6-(2-methoxyvinyl)pyridin-4-yl)(4-(methylsulfonyl)piperazi n-1-yl)methanone (100 g, 0.19 mmol) and 4M hydrochloric acid (dioxane solution, 10 mL) are sequentially added to a reaction flask, and stirred at room temperature for 1 h. When the reaction is completed, the reaction solution is quenched with a saturated sodium bicarbonate aqueous solution (20 mL), and extracted with ethyl acetate (50 mL×2); organic phase is combined, washed with saturated saline (30 mL×2), dried with anhydrous sodium sulfate, and filtered to obtain filtrate; and the filtrate is concentrated under a reduced pressure to obtain 2-(6-(4-(1H-pyrazol-1-yl)phenyl)-4-(4-(methylsulfonyl)piperazin-1-carbonyl)pyridin-2-yl)acetal dehyde with a yield of 82.5%.

ESI-MS(m/z)=472.0 [M+H₂O+H]⁺.

### Step f): Preparation of (2-(4-(1H-pyrazol-1-yl)phenyl)-6-(2-(((1R,2S)-2-(4-fluorophenyl)cyclopropyl)amino)ethyl)pyrid in-4-yl) (4-(methylsulfonyl)piperazin-1-yl)methanone

2-(6-(4-(1H-pyrazol-1-yl)phenyl)-4-(4-(methylsulfonyl)piperazin-1-carbonyl)pyridin-2-yl)acetal dehyde (80 mg, 0.18 mmol), (1R,2S)-2-(4-fluorophenyl)cyclopropan-1-amine (39.6 mg, 0.211 mmol) and 1,2-dichloroethane (10 mL) are added to a reaction flask sequentially, and stirred for 1 h at room temperature; and after a reaction intermediate is generated upon detection, sodium cyanoborohydride (22.2 mg, 0.354 mmol) is added. The reaction solution is stirred for 2 h till the reaction is completed upon detection; the reaction solution is then quenched with a sodium bicarbonate aqueous solution (10 mL), and extracted with ethyl acetate (20 mL×2); organic phase is combined, washed with saturated saline (30 mL×2), dried with anhydrous sodium sulfate, and filtered to obtain filtrate; the filtrate is concentrated under a reduced pressure; and the residue is purified by Prep-HPLC (separation method 4) to obtain (2-(4-(1H-pyrazol-1-yl)phenyl)-6-(2-(((1R,2S)-2-(4-fluorophenyl)cyclopropyl)amino)ethyl)pyrid in-4-yl) (4-(methylsulfonyl)piperazin-1-yl)methanone with a yield of 3.5%.

¹H NMR (400 MHz, Acetonitrile-d₃) δ ppm 8.24 (d, J=2.4 Hz, 2H), 8.22 (s, 1H), 7.96-7.87 (m, 2H), 7.77 (d, J=1.8 Hz, 1H), 7.70 (d, J=1.2 Hz, 1H), 7.20 (d, J=1.2 Hz, 1H), 7.14-7.05 (m, 2H), 7.04-6.96 (m, 2H), 6.57 (t, J=2.2 Hz, 1H), 3.84 (s, 2H), 3.48 (s, 2H), 3.31 (s, 2H), 3.20 (t, J=6.8 Hz, 3H), 3.06 (t, J=7.2 Hz, 2H), 2.84 (s, 3H), 2.38 (ddd, J=7.2, 4.3, 3.1 Hz, 1H), 1.88-1.76 (m, 2H), 1.31 (s, 1H), 1.02-0.86 (m, 2H).

ESI-MS(m/z)=589.0 [M+H]⁺.

### Example 256

### (6-(4-(1H-pyrazol-1-yl)phenyl)-4-(2-(((1R,2S)-2-(4-fluorophenyl)cyclopropyl)amino)ethyl)pyrid in-2-yl) (4-(methylsulfonyl)piperazin-1-yl)methanone is prepared according to the synthesis method of Example 255 (separation method 4), and its structure and characterization data are as follows

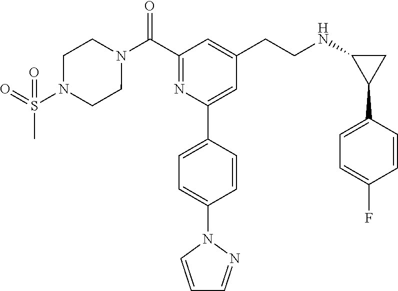 [SMILES: CS(=O)(=O)N1CCN(C(=O)c2cc(CCN[C@@H]3C[C@H]3c3ccc(F)cc3)cc(-c3ccc(-n4cccn4)cc3)n2)CC1]

¹H NMR (400 MHz, DMSO-d₆) δ ppm 8.60 (d, J=2.6 Hz, 1H), 8.22 (d, J=8.6 Hz, 2H), 7.99 (d, J=7.8 Hz, 3H), 7.79 (d, J=1.6 Hz, 1H), 7.46 (s, 1H), 7.15-6.96 (m, 4H), 6.59 (t, J=2.2 Hz, 1H), 3.80 (t, J=5.2 Hz, 2H), 3.67 (t, J=5.0 Hz, 2H), 2.98 (d, J=6.4 Hz, 3H), 2.94 (s, 5H), 2.87 (t, J=7.2 Hz, 2H), 2.46 (s, 1H), 2.31-2.25 (m, 1H), 1.81 (t, J=3.2 Hz, 1H), 0.92 (s, 2H).

ESI-MS(m/z)=589.2 [M+H]⁺.

### Example 257

### Preparation of (2-(4-(1H-pyrazol-1-yl)phenyl)-6-(3-((1R,2S)-2-(4-fluorophenyl)cyclopropyl)amino)pyrimidin-4-yl)(4-(methylsulfonyl)piperazin-1-yl)methanone

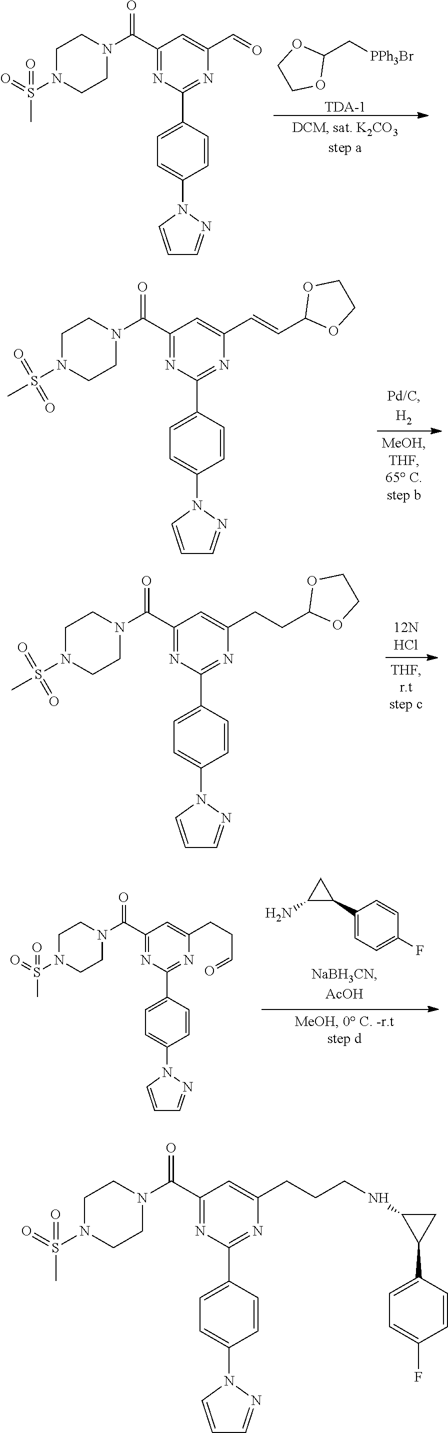

### Step a): Preparation of (E)-(6-(2-(1,3-dioxan-2-yl)vinyl)-2-(4-(1H-pyrazol-1-yl)phenyl)pyrimidin-4-yl)(4-(methylsulfonyl)p iperazin-1-yl)methanone

2-(4-(1H-pyrazol-1-yl)phenyl)-6-(4-(methylsulfonyl)piperazine-1-carbonyl)pyrimidine-4-aminoc arbaldehyde (230 mg, 0.523 mmol), TDA-1 (370 mg, 0.836 mmol), ((1,3-dioxolan-2-yl)methyl)bromotriphenyl-5-phosphine (186 mg, 0.575 mmol), saturated potassium carbonate aqueous solution (10 mL) and dichloromethane (20 ml) are sequentially added to a reaction flask, subjected to nitrogen displacement for three times, warmed to 45° C., and reacted for 2 h while stirring. When the reaction is completed, the reaction solution is quenched with dichloromethane (50 mL×3); organic phase is combined, washed with saturated saline (100 mL×2), and concentrated under a reduced pressure to dryness; and the residue is purified by silica gel chromatography (eluent: petroleum ether/ethyl acetate=1/1) to obtain (E)-(6-(2-(1,3-dioxolan-2-yl)vinyl)-2-(4-(1H-pyrazol-1-yl)phenyl)pyrimidin-4-yl)(4-(methylsulfonyl)piperazin-1-yl)met hanone with a yield of 31.0%.

ESI-MS(m/z)=511.2[M+H]⁺

### Step b): Preparation of (6-(2-(1,3-dioxolan-2-yl)ethyl)-2-(4-(1H-pyrazol-1-yl)phenyl)pyrimidin-4-yl) (4-(methylsulfonyl)piperazin-1-yl)methanone

(E)-(6-(2-(1,3-dioxolan-2-yl)vinyl)-2-(4-(1H-pyrazol-1-yl)phenyl)pyrimidin-4-yl)(4-(methylsulfonyl)piperazin-1-yl)met hanone (83 mg, 0.162 mmol), 5% palladium carbon (10 mg) and methanol (10 mL) are sequentially added to a reaction flask, subjected to hydrogen displacement for three times, and stirred for 2 h at room temperature in a hydrogen atmosphere. When the reaction is completed, the reaction solution is filtered to obtain filtrate; and the filtrate is concentrated under a reduced pressure to obtain (6-(2-(1,3-dioxolan-2-yl)ethyl)-2-(4-(1H-pyrazol-1-yl)phenyl)pyrimidin-4-yl)(4-(methylsulfonyl)piperazin-1-yl)met hanone with a yield of 100%.

ESI-MS m/z)=513.2 [M+H]⁺.

### Step c): Preparation of 3-(2-(4-(1H-pyrazol-1-yl)phenyl)-6-(4-(methylsulfonyl)piperazin-1-carbonyl)pyrimidin-4-yl)pro pionaldehyde

(6-(2-(1,3-dioxolan-2-yl)ethyl)-2-(4-(1H-pyrazol-1-yl)phenyl)pyrimidin-4-yl)(4-(methylsulfonyl) piperazin-1-yl)methanone (83 mg, 0.162 mmol) and tetrahydrofuran (2.5 mL) are sequentially added to a reaction flask, stirred for dissolving, then added with concentrated hydrochloric acid (2.5 mL) and stirred for 30 min at room temperature. When the reaction is completed, the reaction solution is quenched with saturated sodium bicarbonate aqueous solution (20 mL), and extracted with ethyl acetate (20 mL×2); and organic phase is combined, washed with saturated saline (20 mL), dried with anhydrous sodium sulfate, filtered, and concentrated under a reduced pressure to obtain 3-(2-(4-(1H-pyrazol-1-yl)phenyl)-6-(4-(methylsulfonyl)piperazin-1-carbonyl)pyrimidin-4-yl)pro pionaldehyde with a yield of 30.5%.

ESI-MS m/z)=469.2 [M+H]⁺.

### Step d: Preparation of (2-(4-(1H-pyrazol-1-yl)phenyl)-6-(3-((1R,2S)-2-(4-fluorophenyl)cyclopropyl)amino)pyrimidin-4-yl)(4-(methylsulfonyl)piperazin-1-yl)methanone

3-(2-(4-(1H-pyrazol-1-yl)phenyl)-6-(4-(methylsulfonyl)piperazin-1-carbonyl)pyrimidin-4-yl)pro pionaldehyde (23 mg, 0.049 mmol), (1R,2S)-2-(4-fluorophenyl)cyclopropane-1-amine (14 mg, 0.074 mmol), acetic acid (23 uL), methanol (70 uL), and DCE (2 mL) are added to a reaction flask sequentially, subjected to nitrogen displacement for three times, and reacted for 1 h at room temperature while stirring. When the reaction is completed, the reaction solution is then added with sodium cyanoborohydride (21 mg, 0.245 mmol), stirred for 1 h at room temperature till the reaction is ended, quenched with a sodium bicarbonate aqueous solution (20 mL), and extracted with ethyl acetate (20 mL×2); organic phase is combined, washed with saturated saline (20 mL), dried with anhydrous sodium sulfate, filtered, and concentrated under a reduced pressure; and the residue is purified by Prep-HPLC (separation method 4) to obtain (2-(4-(1H-pyrazol-1-yl)phenyl)-6-(3-((1R,2S)-2-(4-fluorophenyl)cyclopropyl)amino)pyrimidin-4-yl)(4-(methylsulfonyl)piperazin-1-yl)methanone with a yield of 20.5%.

¹H NMR (400 MHz, Chloroform-d) δppm 8.51 (d, J=8.4 Hz, 2H), 8.03 (d, J=2.4 Hz, 1H), 7.96-7.73 (m, 3H), 7.39 (s, 1H), 7.13-6.85 (m, 4H), 6.53 (d, J=2.0 Hz, 1H), 3.92 (dt, J=37.8, 5.0 Hz, 4H), 3.40 (dt, J=16.8, 5.0 Hz, 4H), 3.12-2.75 (m, 7H), 2.40-2.28 (m, 1H), 2.08 (t, J=7.4 Hz, 2H), 1.92 (d, J=6.4 Hz, 1H), 1.09 (dq, J=11.4, 6.6, 5.8 Hz, 1H), 0.94 (q, J=6.2 Hz, 1H).

ESI-MS(m/z)=604.2 [M+H]⁺.

### Example 258

Compound is prepared according to the synthesis method of Example 257 (separation method 1), and its structure and characterization data are as follows: 4-(4-(4,4-difluoropiperidine-1-carbonyl)-6-(3-(((1R,2S)-2-(4-fluorophenyl)cyclopropyl)amino)pr opyl)pyrimidin-2-yl)benzonitrile hydrochloride

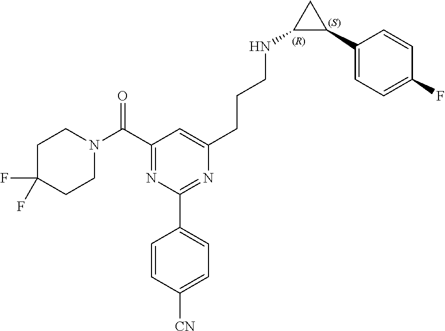

¹H NMR (400 MHz, DMSO-d₆+D₂0) δppm 8.67-8.41 (m, 2H), 8.16-7.94 (m, 2H), 7.65 (s, 1H), 7.22 (ddd, J=8.6, 5.5, 2.8 Hz, 2H), 7.20-7.03 (m, 2H), 3.80 (t, J=6.0 Hz, 2H), 3.56 (d, J=6.2 Hz, 2H), 3.19 (q, J=6.0, 4.0 Hz, 2H), 3.12-2.92 (m, 3H), 2.14 (dtd, J=41.8, 14.8, 14.2, 7.6 Hz, 7H), 1.49 (dd, J=6.4, 4.4 Hz, 1H), 1.30 (dt, J=8.0, 6.4 Hz, 1H).

ESI-MS(m/z)=520.2 [M+H]⁺.

### Example 259

### Preparation of 4-(4-(3-(1R,2S)-2-(4-fluorophenyl)cyclopropyl)amino)-6-(4-(methylsulfonyl)piperazin-1-yl)-1,3,5-triazin-2-yl)benzonitrile

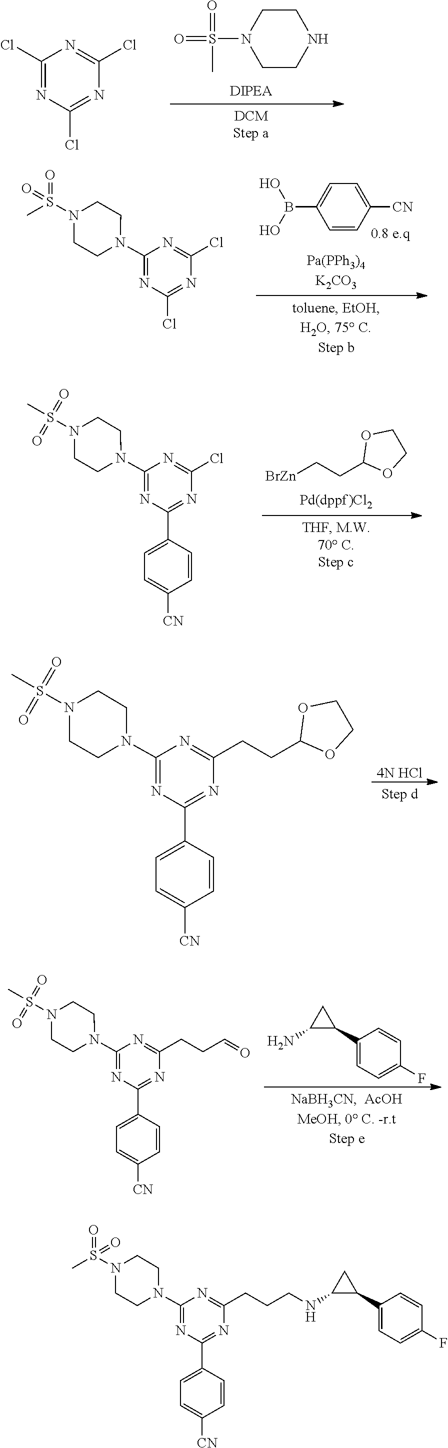

### Step a): Preparation of 2,4-dichloro-6-(4-(methylsulfonyl)piperazin-1-yl)-1,3,5-triazine

2,4,6-trichloro-1,3,5-triazine (3.0 g, 16.393 mmol), 1-(methylsulfonyl)piperazine (5.4 g, 32.787 mmol), DIPEA (2.1 g, 16.393 mmol), and DCM (50 mL) are sequentially added to a reaction flask, subjected to nitrogen displacement for three times, warmed to 50° C. and reacted for 1 h while stirring. When the reaction is completed, the reaction solution is concentrated under a reduced pressure; and the residue is purified by silica gel chromatography (eluent: petroleum ether/ethyl acetate=1/1) to obtain 2,4-dichloro-6-(4-(methylsulfonyl)piperazin-1-yl)-1,3,5-triazine with a yield of 65.3%.

ESI-MS m/z)=312.0 [M+H]⁺.

### Step b): Preparation of 4-(4-chloro-6-(4-(methylsulfonyl)piperazin-1-yl)-1,3,5-triazin-2-yl)benzonitrile

2,4-dichloro-6-(4-(methylsulfonyl)piperazin-1-yl)-1,3,5-triazine (3.3 g, 10.655 mmol), (4-cyanophenyl)boronic acid (1.5 g, 10.655 mmol), K₂CO₃ (2.9 g, 21.310 mmol), Pd(dppf)Cl₂ (780 mg, 1.066 mmol), 1.4-Dioxane (40 mL) and water (10 mL) are sequentially added to a reaction flask, and reacted at 75° C. for 6 h while stirring. When the reaction is completed, the reaction solution is concentrated under a reduced pressure to dryness; and the residue is purified by silica gel chromatography (eluent: petroleum ether/ethyl acetate=1/1) to obtain 4-(4-chloro-6-(4-(methylsulfonyl)piperazin-1-yl)-1,3,5-triazin-2-yl)benzonitrile with a yield of 31.1%.

ESI-MS(m/z)=379.1[M+H]⁺.

### Step c): Preparation of 4-(4-(2-(1,3-dioxolane-2-yl)ethyl)-6-(4-(methylsulfonyl)piperazin-1-yl)-1,3,5-triazin-2-yl)benzo nitrile

4-(4-chloro-6-(4-(methylsulfonyl)piperazin-1-yl)-1,3,5-triazin-2-yl)benzonitrile (1.256 g, 3.313 mmol), (2-(1,3-dioxolan-2-yl)ethyl)zinc bromide (II) (5 mL, 1M, 4.970 mmol), Pd(dppf)Cl₂ (242 mg, 0.331 mmol) and THE (15 mL) are added to a 30 mL microwave tube, subjected to nitrogen displacement for three times and reacted in microwaves at 75° C. for 1 h. When the reaction is completed, the reaction solution is concentrated under a reduced pressure; and the residue is purified by silica gel chromatography (eluent: petroleum ether/ethyl acetate=1/1) to obtain 4-(4-(2-(1,3-dioxolan-2-yl)ethyl)-6-(4-(methylsulfonyl)piperazin-1-yl)-1,3,5-triazin-2-yl)benzoni trile with a yield of 31.5%.

¹H NMR (400 MHz, DMSO-d₆) δppm 8.64-8.46 (m, 2H), 8.14-7.89 (m, 2H), 4.94 (d, J=4.8 Hz, 1H), 4.13-3.74 (m, 8H), 3.24 (d, J=5.8 Hz, 4H), 2.90 (d, J=1.8 Hz, 3H), 2.80 (t, J=7.8 Hz, 2H), 2.10 (q, J=6.8, 6.4 Hz, 2H).

ESI-MS(m/z)=445.2 [M+H]⁺.

### Step d): Preparation of 4-(5-(4-(methylsulfonyl)piperazin-1-yl)-6-oxy-3-(3-oxypropyl)pyridazine-1(6H)-yl)benzonitrile

4-(4-(2-(1,3-dioxolan-2-yl)ethyl)-6-(4-(methylsulfonyl)piperazin-1-yl)-1,3,5-triazin-2-yl)benzoni trile (465 mg, 1.043 mmol), THF (3 mL) and 4N hydrochloric acid (aqueous solution, 3 mL) are added to a reaction flask sequentially and stirred for 2 h at room temperature. When the reaction is completed, the reaction solution is poured into a sodium bicarbonate aqueous solution (20 mL), and extracted with ethyl acetate (20 mL×2); organic phase is combined, washed with saturated saline (10 mL×2), dried with anhydrous sodium sulfate, and filtered to obtain filtrate; the filtrate is concentrated under a reduced pressure; and the residue is purified by silica gel chromatography (eluent: petroleum ether/ethyl acetate=1/1) to obtain 4-(5-(4-(methylsulfonyl)piperazin-1-yl)-6-oxy-3-(3-oxypropyl)pyridazine-1(6H)-yl)benzonitrile with a yield of 45.5%.

ESI-MS m/z)=401.1 [M+H]⁺.

### Step e): Preparation of 4-(4-(3-(1R,2S)-2-(4-fluorophenyl)cyclopropyl)amino)-6-(4-(methylsulfonyl)piperazin-1-yl)-1,3,5-triazin-2-yl)benzonitrile

4-(5-(4-(methylsulfonyl)piperazin-1-yl)-6-oxy-3-(3-oxypropyl)pyridazine-1(6H)-yl)benzonitrile (190 mg, 0.475 mmol), (1R,2S)-2-(4-fluorophenyl)cyclopropane-1-amine (89 mg, 0.475 mmol), acetic acid (190 uL), methanol (950 uL), and DCE (5 mL) are sequentially added to a reaction flask, subjected to nitrogen displacement for three times, and reacted for 1 h at room temperature while stirring. When the reaction is completed, the reaction solution is then added with sodium cyanoborohydride (61 mg, 0.950 mmol), stirred for 1 h at room temperature till the reaction is ended, quenched with a sodium bicarbonate aqueous solution (20 mL), and extracted with ethyl acetate (20 mL×2); organic phase is combined, washed with saturated saline (20 mL), dried with anhydrous sodium sulfate, filtered, and concentrated under a reduced pressure; and the residue is purified by Prep-HPLC (separation method 3) to obtain 4-(4-(3-(1R,2S)-2-(4-fluorophenyl)cyclopropyl)amino)-6-(4-(methylsulfonyl)piperazin-1-yl)-1,3,5-triazin-2-yl)benzonitrile with a yield of 41.5%.

¹H NMR (400 MHz, DMSO-d₆) δppm 8.90-8.32 (m, 2H), 8.01 (d, J=8.2 Hz, 2H), 7.03 (qd, J=9.4, 4.5 Hz, 4H), 4.02 (d, J=40.8 Hz, 4H), 3.23 (t, J=6.6 Hz, 4H), 2.90 (d, J=2.4 Hz, 3H), 2.72 (dt, J=18.4, 7.4 Hz, 4H), 2.19 (dt, J=6.8, 3.4 Hz, 1H), 1.97-1.84 (m, 2H), 1.80 (td, J=6.2, 3.0 Hz, 1H), 1.03-0.58 (m, 2H).

ESI-MS(m/z)=536.2 [M+H]⁺.

### Example 260

### Preparation of 4-(3-(3-((1R,2S)-2-(4-fluorophenyl)cyclopropyl)amino)-5-(4-(methylsulfonyl)piperazin-1-yl)-6-oxypyridazine-1 (6H)-yl)benzonitrile formate

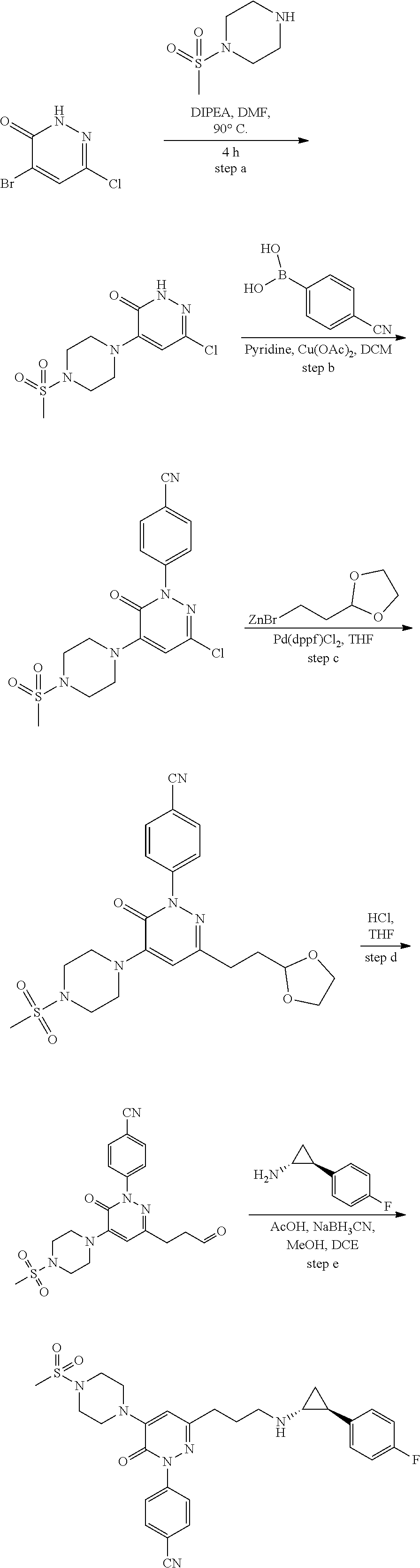

### Step a): Preparation of 6-chloro-4-(4-(methylsulfonyl)piperazin-1-yl)pyridinazine-3(2H)-one

4-bromo-6-chloropyridine-3(2H)-one (5 g, 23.8 mmol), and 1-(methylsulfonyl)piperazine (3.9 g, 23.8 mol) are dissolved in DMF (30 mL), added with DIPEA (9.2 g, 71.4 mmol) in an ice bath, warmed to 90° C. and reacted for 4 h. When LC-MS monitors that raw materials is reacted completely, the reaction solution is cooled to room temperature, then quenched with water (20 mL), and extracted with ethyl acetate (20 mL×2); organic phase is combined, washed with saturated saline (15 mL×2), dried with anhydrous sodium sulfate, filtered and concentrated under a reduced pressure; and the residue is purified by silica gel chromatography (eluent: petroleum ether/ethyl acetate=10/3) to obtain 6-chloro-4-(4-(methylsulfonyl)piperazin-1-yl) pyridinyl)pyridazine-3(2H)-one with a yield of 82.3%.

¹H NMR (400 MHz, DMSO-d₆) δppm 12.82 (s, 1H), 6.57 (s, 1H), 3.66 (t, J=5.0 Hz, 4H), 3.20 (t, J=5.0 Hz, 4H), 2.91 (s, 3H).

ESI-MS m/z=293.0 [M+H]⁺.

### Step b): Preparation of 4-(3-chloro-5-(4-hydrosulfonylpiperazin-1-yl)-6-oxypyridine-1(6H)benzonitrile

6-chloro-4-(4-(methylsulfonyl)piperazin-1-yl)pyridin-3(2H)-one (3 g, 10.2 mmol), (4-cyanophenyl)boronic acid (1.5 g, 10.2 mmol), Cu(OAc)₂ (372 mg, 2.0 mmol), pyridine (2.4 g, 30.6 mmol) and DCM (20 mL) are sequentially added to a reaction flask and reacted for 12 h at room temperature. When LC-MS shows that raw materials are reacted completely, the reaction solution is quenched with water (20 mL), and extracted with ethyl acetate (20 mL×2); organic phase is combined, washed with saturated saline (15 mL×2), dried with anhydrous sodium sulfate, filtered and concentrated under a reduced pressure; and the residue is purified by silica gel chromatography (eluent: petroleum ether/ethyl acetate=5/2) to obtain 4-(3-chloro-5-(4-hydrosulfonylpiperazin-1-yl)-6-oxypyridine-1(6H)benzonitrile with a yield of 61.5%.

ESI-MS m/z=394.1 [M+H]⁺.

### Step c): Preparation of 4-(3-(2-(1,3-dioxolan-2-yl)ethyl)-5-(4-(methylsulfonyl)piperazin-1-yl)-6-oxypyridine-1(6H)yl)benzonitrile

4-(3-chloro-5-(4-hydrosulfonylpiperazin-1-yl)-6-oxypyridine-1(6H)yl)benzonitrile (400 mg, 1.05 mmol), 2-(1,3-dioxolan-2-yl)ethyl)zinc bromide (778 mg, 3.15 mmol), and Pd(dppf)Cl₂ (77 mg, 0.105 mmol) are added to a microwave tube, blown with nitrogen continuously for 1 min and reacted in microwaves at 70° C. for 1 h. When LC-MS shows that the reaction has been completed, the reaction solution is concentrated under a reduced pressure; and the residue is purified by silica gel chromatography (eluent: petroleum ether/ethyl acetate=5/3) to obtain 4-(3-(2-(1,3-dioxolan-2-yl)ethyl)-5-(4-(methylsulfonyl)piperazin-1-yl)-6-oxypyridine-1(6H)yl)benzonitrile with a yield of 34.5%.

ESI-MS m/z=460.1 [M+H]⁺.

### Step d): Preparation of 4-(5-(4-(methylsulfonyl)piperazin-1-yl)-6-oxo-3-(3-oxypropyl)pyridazine-1(6H)-yl)benzonitrile

4-(3-(2-(1,3-dioxolan-2-yl)ethyl)-5-(4-(methylsulfonyl)piperazin-1-yl)-6-oxypyridine-1(6H)yl)b enzonitrile (140 mg, 0.33 mmol) is dissolved in THE (1 mL), added with a 4N hydrochloric acid (aqueous solution, 1 mL) and reacted at room temperature for 2 h. When LC-MS shows that the reaction is completed, the reaction solution is concentrated directly to obtain a crude product 4-(5-(4-(methylsulfonyl)piperazin-1-yl)-6-oxo-3-(3-oxypropyl)pyridazine-1(6H)-yl)benzonitrile; and the crude product is used directly in the next step.

ESI-MS m/z=416.2 [M+H]⁺.

### Step e): Preparation of 4-(3-(3-(((1R,2S)-2-(4-fluorophenyl)cyclopropyl)amino)propyl)-5-(4-(methylsulfonyl)piperazin-1-yl)-6-oxypyridazine-1 (6H)-yl)benzonitrile carboxylate

4-(5-(4-(methylsulfonyl)piperazin-1-yl)-6-oxo-3-(3-oxypropyl)pyridazine-1(6H)-yl)benzonitrile (60 mg, 0.14 mmol) and (1R,2S)-2-(4-fluorophenyl)cyclopropane-1-amine (27.8 mg, 0.14 mmol) are dissolved in DCE (2 mL), then added with AcOH (0.1 mL), MeOH (0.5 mL), and NaBH₃CN (36 mg, 0.57 mmol), subjected to nitrogen displacement for three times and then reacted at room temperature for 2 h. When LC-MS shows that the reaction has been completed, the reaction solution is quenched with a sodium bicarbonate aqueous solution (20 mL), and extracted with ethyl acetate (20 mL×2); and organic phase is combined, washed with saturated saline (15 mL×2), dried with anhydrous sodium sulfate, filtered, concentrated under a reduced pressure, and purified by Prep-HPLC (separation method 3) to obtain 4-(3-(3-(((1R,2S)-2-(4-fluorophenyl)cyclopropyl)amino)propyl)-5-(4-(methylsulfonyl)piperazin-1-yl)-6-oxypyridazine-1 (6H)-yl)benzonitrile formate with a yield of 5.9%.

¹H NMR (400 MHz, Methanol-d₄) δ ppm 8.50 (s, H), 7.87-7.82 (m, 2H), 7.79 (d, J=8.0 Hz, 2H), 7.12-7.05 (m, 2H), 6.98 (td, J=8.8, 2.4 Hz, 2H), 6.63 (d, J=2.6 Hz, 1H), 3.60-3.53 (m, 4H), 3.43-3.35 (m, 4H), 2.95 (t, J=7.8 Hz, 2H), 2.90 (d, J=2.6 Hz, 3H), 2.72 (t, J=7.6 Hz, 2H), 2.51 (dd, J=7.6, 3.8 Hz, 1H), 2.10-1.97 (m, 3H), 1.19 (s, 1H), 1.10 (d, J=6.8 Hz, 1H).

ESI-MS m/z=551.2 [M+H]⁺.

### Example 261

### Preparation of 4-(4-(3-(1R,2S)-2-(4-fluorophenyl)cyclopropyl)amino)-6-(4-(methylsulfonyl)piperazin-1-yl)pyri midin-2-yl)benzonitrile formate

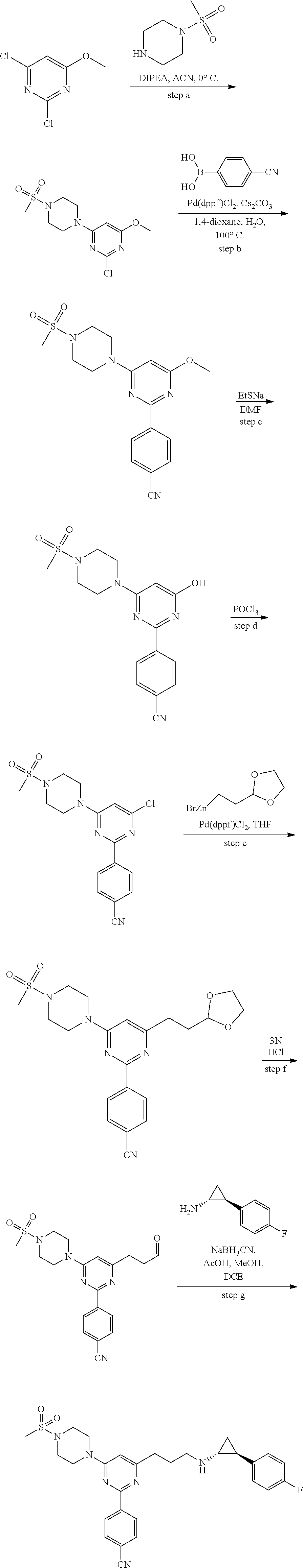

### Step a): Preparation of 2-chloro-4-methoxy-6-(4-(methylsulfonyl)piperazin-1-yl)pyrimidine

2,4-dichloro-6-methoxypyrimidine (2.3 g.12.9 mmol), 1-(methylsulfonyl)piperazine (2.1 g.12.9 mmol), and DIPEA (3.3 g, 25.8 mmol) are dissolved in acetonitrile (90 mL). The solution is reacted for 4 h at 0° C. When LS-MS shows the reaction is completed, the reaction solution is quenched with water (200 mL), and extracted with ethyl acetate (200 mL×2); organic phase is combined, washed with saturated saline (150 mL×2), dried with anhydrous sodium sulfate, filtered and concentrated to obtain a crude product; and the residue is purified by silica gel chromatography (eluent: petroleum ether/ethyl acetate=10/7) to obtain 2-chloro-4-methoxy-6-(4-(methylsulfonyl)piperazin-1-yl)pyrimidine with a yield of 60.2%.

¹H NMR (400 MHz, DMSO-d₆) δ ppm 6.24 (s, 1H), 3.91-3.77 (m, 7H), 3.18 (t, J=5.2 Hz, 4H), 2.89 (s, 3H).

ESI-MS m/z=307.1 [M+H]⁺.

### Step b): Preparation of 4-(4-methoxy-6-(4-(methylsulfonyl)piperazin-1-yl)pyrimidin-2-yl)benzonitrile

2—Chloro-4-methoxy-6-(4-(methylsulfonyl)piperazin-1-yl)pyrimidine (1.0 g, 3.25 mmol), (4-cyanophenyl)boronic acid (575 mg, 3.91 mmol), Cs₂CO₃ (2.124 mg, 6.52 mmol), Pd(dppf)Cl₂(238 mg, 0.325 mmol), 15 mL of dioxane and 1.5 mL of water are sequentially added to a reaction flask to obtain a mixed solution; the mixed solution is purged with by nitrogen blowing, and reacted in a microwave reactor at 100° C. for 60 min; and LC-MS shows that the reaction is completed, a crude product is obtained by concentration; and the residue is purified by silica gel chromatography (eluent: petroleum ether/ethyl acetate=10/9) to obtain 4-(4-methoxy-6-(4-(methylsulfonyl)piperazin-1-yl)pyrimidin-2-yl)benzonitrile with a yield of 97.2%.

¹H NMR (400 MHz, DMSO-d₆) δ ppm 8.29 (d, J=8.0 Hz, 2H), 7.96 (d, J=8.2 Hz, 2H), 6.82 (s, 1H), 3.95 (d, J=15.2 Hz, 7H), 3.46-3.11 (m, 4H), 2.90 (s, 3H).

ESI-MS m/z=374.7 [M+H]⁺.

### Step c): Preparation of 4-(4-hydroxy-6-(4-(methylsulfonyl)piperazin-1-yl)pyrimidin-2-yl)benzonitrile

Ethanethiol (5 mL) is dissolved in N,N-dimethylformamide (20 mL), added with NaH (4 g, 60% purity) at room temperature, and stirred for 30 min to obtain sodium ethanethiolate solution; 4-(4-methoxy-6-(4-(methylsulfonyl)piperazin-1-yl)pyrimidin-2-yl)benzonitrile (1.5 g, 4.02 mmol) is dissolved in a sodium ethanethiolate solution (20 mL), and reacted at 60° C. for 60 min; when the reaction is completed, the reaction solution is dried directly by pumping with an oil pump; and the residue is purified by silica gel chromatography (eluent: dichloromethane/methanol=5/2) to obtain 4-(4-hydroxy-6-(4-(methylsulfonyl)piperazin-1-yl)pyrimidin-2-yl)benzonitrile with a yield of 89.2%.

ESI-MS m/z=360.1 [M+H]⁺.

### Step d): Preparation of 4-(4-chloro-6-(4-methylsulfo)piperazin-1-yl)pyrimidin-2-yl)benzonitrile

4-(4-hydroxy-6-(4-(methylsulfonyl)piperazin-1-yl)pyrimidin-2-yl)benzonitrile (1.5 g, 4.02 mmol) is dissolved in phosphorus oxychloride (10 mL), reacted at 100° C. for 12 h, and concentrated to obtain a crude product; and the residue is purified by silica gel chromatography (eluent: petroleum ether/ethyl acetate=10/9) to obtain 4-(4-chloro-6-(4-methylsulfonyl)piperazin-1-yl)pyrimidin-2-yl)benzonitrile with a yield of 95.2%.

ESI-MS m/z:378.1 [M+H]⁺.

### Step e): Preparation of 4-(4-(2-(1,3-dioxolan-2-yl)ethyl)-6-(4-(methylsulfonyl)piperazin-1-yl)pyrimidin-2-yl)benzonitril e

4-(4-chloro-6-(4-methylsulfonyl)piperazin-1-yl)pyrimidin-2-yl)benzonitrile (100 mg, 0.265 mmol), 2-(1,3-dioxolan-2-yl)ethyl)zinc bromide (1.6 mL, 0.795 mmol), Pd(dppf)Cl₂ (20 mg, 0.0265 mmol) and tetrahydrofuran (10 mL) are added sequentially to a reaction flask. The solution is reacted for 1.5 h at 100° C., and LS-MS shows the reaction is completed. The reaction solution is concentrated to obtain a crude product; and the residue is purified by silica gel chromatography (eluent: petroleum ether/ethyl acetate=10/9) to obtain 4-(4-(2-(1,3-dioxolan-2-yl)ethyl)-6-(4-(methylsulfonyl)piperazin-1-yl)pyrimidin-2-yl)benzonitrile with a yield of 83.5%.

ESI-MS m/z:444.2 [M+H]⁺.

### Step f): Preparation of 4-(4-(4-methylsulfonyl)piperazin-1-yl)-6-(3-oxopropyl)pyrimidin-2-yl)benzonitrile

4-(4-(2-(1,3-dioxolan-2-yl)ethyl)-6-(4-(methylsulfonyl)piperazin-1-yl)pyrimidin-2-yl)benzonitril e (100 mg, 0.225 mmol) is added to a 2M dioxane hydrochloride solution (5 mL) and stirred for 1 h; when LS-MS shows that the reaction is completed, the reaction solution is added with a saturated sodium bicarbonate solution to adjust pH to 8-9 in an ice bath, and extracted with ethyl acetate (20 mL×2); the organic phase is combined, washed with saturated saline (15 mL×2), dried with anhydrous sodium sulfate, filtered, and concentrated to obtain a crude product; and the residue is purified by silica gel chromatography (eluent: petroleum ether/ethyl acetate=10/7) to obtain 4-(4-(4-methylsulfonyl)piperazin-1-yl)-6-(3-oxopropyl)pyrimidin-2-yl)benzonitrile with a yield of 43.4%.

ESI-MS m/z:400.1 [M+H]⁺.

### Step g): Preparation of 4-(4-(3-(1R,2S)-2-(4-fluorophenyl)cyclopropyl)amino)propyl)-6-(4-(methylsulfonyl)piperazin-1-yl)pyrimidin-2-yl)benzonitrile formate

4-(4-(4-methylsulfonyl)piperazin-1-yl)-6-(3-oxypropyl)pyrimidin-2-yl)benzonitrile (56 mg, 0.403 mmol) is dissolved in DCE (1 mL), then added with MeOH (0.2 mL), AcOH (0.01 mL) and (1R,2S)-2-(4-fluorophenyl)cyclopropylamine (26 mg, 0.1403 mmol), and stirred for 2 h at room temperature, and TLC shows that the raw materials are consumed completely. The reaction solution is added with NaBH₃CN(35 mg, 0.561 mmol); when TLC and LC-MS show that the raw materials are consumed completely, the mixture is quenched with water, and concentrated to obtain a crude product; and the crude product is purified by Prep-HPLC (separation method 3) to obtain 4-(4-(3-(1R,2S)-2-(4-fluorophenyl)cyclopropyl)amino)propyl)-6-(4-(methylsulfonyl)piperazin-1-yl)pyrimidin-2-yl)benzonitrile formate with a yield of 5.4%.

¹H NMR (400 MHz, Methanol-d₄) δ ppm 8.52 (s, 1H), 8.27 (d, J=8.4 Hz, 2H), 7.93-7.71 (m, 2H), 7.22-6.85 (m, 5H), 4.03 (t, J=4.8 Hz, 4H), 3.35-3.16 (m, 4H), 3.01-2.71 (m, 7H), 2.48 (dd, J=7.0, 3.8 Hz, 1H), 2.06 (q, J=7.4 Hz, 3H), 1.41-0.89 (m, 2H).

ESI-MS m/z:535.2[M+H]⁺.

### Example 262

### Preparation of (2-(4-(1H-pyrazol-1-yl)phenyl)-6-(4-((1R,2S)-2-(4-fluorophenyl)cyclopropyl)amino)butyl)pyrim idin-4-yl(4-(methylsulfonyl)piperazin-1-yl)methanone formate

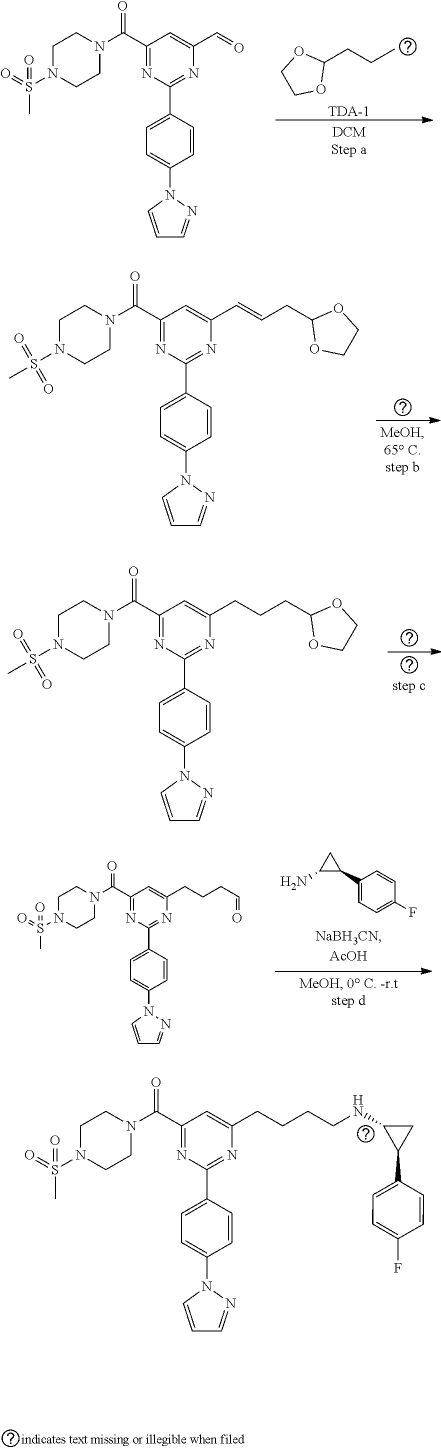

### Step a): Preparation of (E)-(6-(3-(1,3-dioxolan-2-yl)propyl-1-en-1-yl)-2-(4-(1H-pyrazol-1-yl)phenyl)pyrimidin-4-yl)(4-(met hylsulfonyl)piperazin-1-yl)methanone

2-(4-(1H-pyrazol-1-yl)phenyl)-6-(4-(methylsulfonyl)piperazine-1-carbonyl)pyrimidine-4-aminoc arbaldehyde (200 mg, 0.455 mmol), TDA-1 (220 mg, 0.682 mmol), (2-(1,3-dioxolan-2-yl)methyl)bromotriphenyl-15-phosphine (206 mg, 0.575 mmol), saturated potassium carbonate aqueous solution (10 mL) and dichloromethane (20 mL) are sequentially added to a reaction flask, subjected to nitrogen displacement for three times, warmed to 45° C., and reacted for 2 h while stirring. When the reaction is completed, the reaction solution is quenched with dichloromethane (50 mL×3); organic phase is combined, washed with saturated saline (50 mL×2), and concentrated under a reduced pressure to dryness; and the residue is purified by silica gel chromatography (eluent: petroleum ether/ethyl acetate=1/2) to obtain (E)-(6-(3-(1,3-dioxolan-2-yl)propyl-1-en-1-yl)-2-(4-(1H-pyrazol-1-yl)phenyl)pyrimidin-4-yl)(4-(met hylsulfonyl)piperazin-1-yl)methanone with a yield of 30.0%.

ESI-MS(m/z)=525.2[M+H]⁻

### Step b): Preparation of (6-(3-(1,3-dioxolan-2-yl)propyl)-2-(4-(1H-pyrazol-1-yl)phenyl)pyrimidin-4-yl) (4-(methylsulfonyl)piperazin-1-yl)methanone

(E)-(6-(3-(1,3-dioxolan-2-yl)propyl-1-en-1-yl)-2-(4-(1H-pyrazol-1-yl)phenyl)pyrimidin-4-yl)(4-(methylsulfonyl)piperazin-1-yl)methanone (72 mg, 0.137 mmol), 5% palladium carbon (10 mg) and methanol (10 mL) are sequentially added to a reaction flask, subjected to hydrogen displacement for three times, and stirred for 2 h at room temperature in a hydrogen atmosphere. When the reaction is completed, the reaction solution is filtered to obtain filtrate; the filtrate is concentrated under a reduced pressure to obtain (6-(3-(1,3-dioxolan-2-yl)propyl)-2-(4-(1H-pyrazol-1-yl)phenyl)pyrimidin-4-yl) (4-(methylsulfonyl)piperazin-1-yl)methanone with a yield of 100%.

ESI-MS m/z)=527.2 [M+H]⁺.

### Step c): Preparation of 4-(2-(4-(1H-pyrazol-1-yl)phenyl)-6-(4-(methylsulfonyl)piperazin-1-carbonyl)pyrimidin-4-yl)but yraldehyde

(6-(3-(1,3-dioxolan-2-yl)propyl)-2-(4-(1H-pyrazol-1-yl)phenyl)pyrimidin-4-yl)(4-(methylsulfon yl)piperazin-1-yl)methanone (72 mg, 0.137 mmol) and tetrahydrofuran (2.5 mL) are sequentially added to a reaction flask, stirred for dissolving, then added with concentrated hydrochloric acid (2.5 mL) and stirred for 30 min at room temperature. When the reaction is completed, the reaction solution is quenched with a saturated sodium bicarbonate aqueous solution (20 mL), and extracted with ethyl acetate (20 mL×2); organic phase is combined, washed with saturated saline (20 mL), dried with anhydrous sodium sulfate, filtered and concentrated under a reduced pressure to obtain 4-(2-(4-(1H-pyrazol-1-yl)phenyl)-6-(4-(methylsulfonyl)piperazin-1-carbonyl)pyrimidin-4-yl)but yraldehyde with a yield of 32.0%.

ESI-MS(m/z)=483.2[M+H]⁺.

### Step d): Preparation of (2-(4-(1H-pyrazol-1-yl)phenyl)-6-(4-((1R,2S)-2-(4-fluorophenyl)cyclopropyl)amino)butyl)pyrim idin-4-yl(4-(methylsulfonyl)piperazin-1-yl)methanone formate

4-(2-(4-(1H-pyrazol-1-yl)phenyl)-6-(4-(methylsulfonyl)piperazin-1-carbonyl)pyrimidin-4-yl)but yraldehyde (21 mg, 0.044 mmol), (1R,2S)-2-(4-fluorophenyl)cyclopropane-1-amine (12 mg, 0.066 mmol), acetic acid (21 uL), methanol (80 uL), and DCE (2 mL) are added to a reaction flask sequentially, subjected to nitrogen displacement for three times, and reacted for 1 h at room temperature while stirring. When the reaction is completed, the reaction solution is then added with sodium cyanoborohydride (18 mg, 0.220 mmol), stirred for 1 h at room temperature till the reaction is ended, quenched with a sodium bicarbonate aqueous solution (20 mL), and extracted with ethyl acetate (20 mL×2); organic phase is combined, washed with saturated saline (20 mL), dried with anhydrous sodium sulfate, filtered, and concentrated under a reduced pressure; and the residue is purified by Prep-HPLC (separation method 3) to obtain (2-(4-(1H-pyrazol-1-yl)phenyl)-6-(4-((1R,2S)-2-(4-fluorophenyl)cyclopropyl)amino)butyl)pyrim idin-4-yl(4-(methylsulfonyl)piperazin-1-yl)methanone formate with a yield of 22.5%.

¹H NMR (400 MHz, DMSO-d₆) δppm 8.62 (d, J=2.4 Hz, 1H), 8.50 (m, 3H), 8.12-7.92 (m, 2H), 7.82 (d, J=1.8 Hz, 1H), 7.50 (s, 1H), 7.02 (dd, J=7.4, 3.8 Hz, 4H), 6.61 (t, J=2.0 Hz, 1H), 3.80 (t, J=5.0 Hz, 2H), 3.60 (t, J=4.8 Hz, 2H), 3.28-3.12 (m, 4H), 2.95 (s, 3H), 2.87 (t, J=7.6 Hz, 2H), 2.65 (t, J=7.0 Hz, 2H), 2.16 (dd, J=7.0, 3.6 Hz, 1H), 1.90-1.69 (m, 3H), 1.50 (t, J=7.4 Hz, 2H), 0.91 (ddd, J=14.6, 8.0, 4.8 Hz, 2H).

ESI-MS(m/z)=618.3 [M+H]⁺.

### Example 263

### Preparation of 4-(4-((((1R,2S)-2-(4-fluorophenyl)cyclopropyl)amino)methyl)-6-(4-methylpiperazin-1-yl)pyrimi din-2-yl)benzonitrile

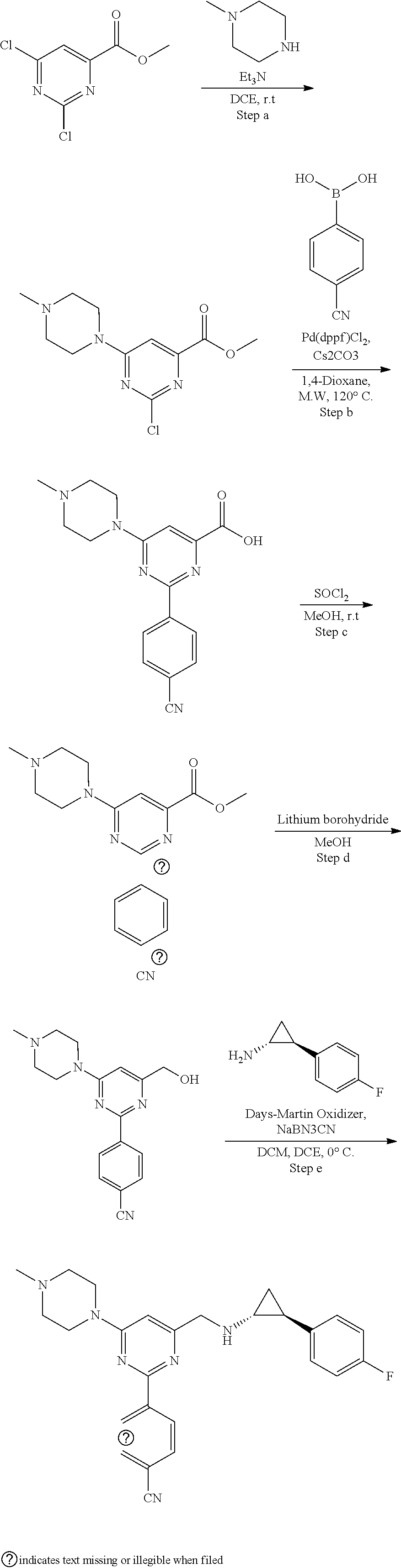

### Step a): Preparation of methyl 2-chloro-6-(4-methylpiperazin-1-yl)pyrimidine-4-carboxylate

Methyl 2,6-dichloropyrimidine-4-carboxylate (1.0 g, 4.83 mmol) and 1-methylpiperazine (0.48 g, 4.83 mol) are dissolved in DMF (15 mL), added with Et₃N (0.48 g, 4.83 mmol) in an ice bath, and reacted at room temperature for 30 min. The reaction solution is cooled to room temperature, then quenched with water (20 mL), and extracted with ethyl acetate (20 mL×2); organic phase is combined, washed with saturated saline (15 mL×2), dried with anhydrous sodium sulfate, filtered and concentrated under a reduced pressure; and the residue is purified by silica gel chromatography (eluent: petroleum ether/ethyl acetate=10/3) to obtain methyl 2-chloro-6-(4-methylpiperazin-1-yl)pyrimidine-4-carboxylate with a yield of 81.4%.

¹H NMR (400 MHz, DMSO-d₆) δppm 7.33 (s, 1H), 3.86 (s, 3H), 3.72 (d, J=24.6 Hz, 4H), 2.38 (t, J=5.2 Hz, 4H), 2.21 (s, 3H).

ESI-MS m/z=271.1 [M+H]⁺.

### Step b): Preparation of 2-(4-cyanophenyl)-6-(4-methylpiperazin-1-yl)pyrimidine-4-carboxylic acid

Methyl 2-chloro-6-(4-methylpiperazin-1-yl)pyrimidine-4-carboxylate (1.18 g, 4.36 mmol), (4-cyanophenyl)boronic acid (0.77 g, 5.23 mmol), Pd(dppf)Cl₂ (0.32 g, 0.44 mmol) and cesium carbonate (2.84 g, 8.72 mmol) are added to a microwave tube containing 1,4-dioxane (20 mL) and water (5 mL), and the mixture is stirred at 120° C. in microwaves for 1 h. When the reaction solution is cooled to room temperature, the reaction solution is added with silica gel and concentrated under a reduced pressure; and the residue is purified by silica gel chromatography (eluent: petroleum ether/ethyl acetate=2/1) to obtain 2-(4-cyanophenyl)-6-(4-methylpiperazin-1-yl)pyrimidine-4-carboxylic acid with a yield of 85.12%.

ESI-MS m/z=324.2 [M+H]⁺.

### Step c): Preparation of methyl 2-(4-cyanophenyl)-6-(4-methylpiperazin-1-yl)pyrimidin-4-carboxylate

Thionyl chloride (0.74 g, 6.18 mmol) and 2-(4-cyanophenyl)-6-(4-methylpiperazin-1-yl)pyrimidin-4-carboxylic acid (1 g, 3.09 mmol) are added to a reaction flask containing methanol (2 mL), the mixture is stirred at 40° C. for 12 h, and a solvent is concentrated to obtain methyl 2-(4-cyanophenyl)-6-(4-methylpiperazin-1-yl)pyrimidin-4-carboxylate.

ESI-MS m/z=338.2 [M+H]⁺.

### Step d): Preparation of 4-(4-(hydroxymethyl)-6-(4-methylpiperazin-1-yl)pyrimidin-2-yl)benzonitrile

Methyl 2-(4-cyanophenyl)-6-(4-methylpiperazin-1-yl)pyrimidine-4-carboxylate (220 mg, 0.65 mmol) and lithium borohydride (0.028 g, 1.29 mmol) are added to a reaction flask containing methanol (10 mL), and the mixture is stirred at room temperature for 2 h. The reaction solution is quenched with water (20 mL), and extracted with ethyl acetate (20 mL×2); organic phase is combined, washed with saturated saline (15 mL×2), dried with anhydrous sodium sulfate, and filtered; and the residue is purified by silica gel chromatography (eluent: petroleum ether/ethyl acetate=2/1) to obtain 4-(4-(hydroxymethyl)-6-(4-methylpiperazin-1-yl)pyrimidin-2-yl)benzonitrile with a yield of 57%.

ESI-MS m/z=310.2 [M+H]⁺.

### Step e): Preparation of 4-(4-((((1R,2S)-2-(4-fluorophenyl)cyclopropyl)amino)methyl)-6-(4-methylpiperazin-1-yl)pyrimi din-2-yl)benzonitrile

4-(4-(hydroxymethyl)-6-(4-methylpiperazin-1-yl)pyrimidin-2-yl)benzonitrile (150 mg, 0.48 mmol) and Dess-Martin reagent (0.41 g, 0.96 mmol) are added to a reaction flask containing methanol (5 mL); the mixture is stirred for 2 h at room temperature; the reaction solution is quenched with water (10 mL), and extracted with ethyl acetate (15 mL×2); and organic phase is combined, washed with saturated saline (15 mL×2), dried with anhydrous sodium sulfate, filtered, and concentrated. The reaction solution is then added with (1R,2S)-2-(4-fluorophenyl)cyclopropane-1-amine (0.14 g, 0.72 mmol) and 1,2-dichloroethane (4 mL); the mixture is stirred for 1 h at room temperature, then cooled to 0° C. and added with sodium cyanoborohydride (0.12 g, 1.92 mmol); the mixture is stirred at room temperature for 2 h while stirring; the reaction solution is quenched with water (20 mL), and extracted with ethyl acetate (20 mL×2); organic phase is combined, washed with saturated saline (15 mL×2), dried with anhydrous sodium sulfate, filtered, concentrated and then delivered to Prep-PLC (separation method 4) for freeze-drying to obtain 4-(4-((((1R,2S)-2-(4-fluorophenyl)cyclopropyl)amino)methyl)-6-(4-methylpiperazin-1-yl)pyrimi din-2-yl)benzonitrile with a yield of 3.7%.

¹H NMR (400 VUz, DMSO-d6) δppm 8.47 (dd, J=8.4, 2.2 Hz, 2H), 7.91 (dd, J=8.4, 2.2 Hz, 2H), 7.02 (dt, J=8.6, 2.6 Hz, 4H), 6.80 (d, J=2.2 Hz, 1H), 3.77 (d, J=2.2 Hz, 2H), 3.68 (s, 4H), 2.38 (d, J=5.2 Hz, 4H), 2.26 (q, J=3.6 Hz, 1H), 2.22 (d, J=2.2 Hz, 3H), 1.87 (ddt, J=9.0, 5.6, 2.6 Hz, 1H), 1.08-1.00 (m, 1H), 0.94 (q, J=6.0, 5.2 Hz, 1H).

ESI-MS m/z=443.2 [M+H]⁺.

### Examples 264-265

Compounds are prepared according to the synthesis method of Example 263 (separation method 4and their structures and characterization data are as follows:

| | | | | MS |
|---|---|---|---|---|
| Examples | Chemical name | Structure | ¹H NMR | (M + H)⁺ |
| 264 | 4-(4-((((1R,2S)-2-(4-fluoro- phenyl)cyclopropyl)amino) methyl)-6-(4-(methyl sulfonyl)piperazin-1-yl) pyrimidin-2-yl)benzonitrile | 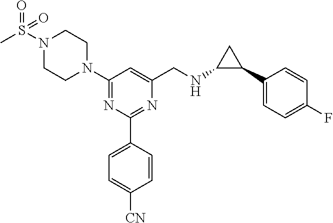 [SMILES: CS(=O)(=O)N1CCN(c2cc(CN[C@@H]3C[C@H]3c3ccc(F)cc3)nc(-c3ccc(C#N)cc3)n2)CC1] | ¹H NMR (400 MHz, DMSO-d₆) δ 8.53-8.46 (m, 2H), 7.98-7.91 (m, 2H), 7.11-6.98 (m, 4H), 6.90 (s, 1H), 3.84 (d, J = 5.2 Hz, 4H), 3.81 (s, 2H), 3.23 (t, J = 5.2 Hz, 4H), 3.11 (s, 1H), 2.91 (s, 3H), 1.89 (ddd, J = 9.2, 5.8, 3.0 Hz, 1H), 1.05 (dt, J = 9.2, 4.6 Hz, 1H), 0.95 (dt, J = 7.0, 5.2 Hz, 1H). | 507.2 |
| 265 | (1R,2S)-2-(4-fluorophenyl)- N-((6-(4-(methylsulfonyl) piperazin-1-yl)-2-(4- morpholinophenyl) pyrimidin-4-yl)methyl) cyclopropane-1-amine | 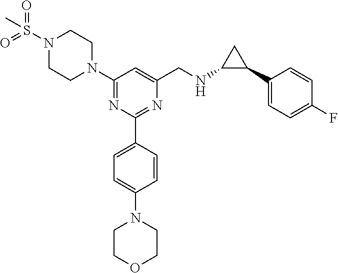 [SMILES: CS(=O)(=O)N1CCN(c2cc(CN[C@@H]3C[C@H]3c3ccc(F)cc3)nc(-c3ccc(N4CCOCC4)cc3)n2)CC1] | ¹H NMR (400 MHz, DMSO-d₆) δ 8.24-8.16 (m, 2H), 7.10-6.93 (m, 6H), 6.70 (s, 1H), 3.86 (m, 4H), 3.68 (m, 6H), 3.32 (s, 8H), 3.26-3.11 (m, 3H), 2.36-2.27 (m, 1H), 1.91 (s, 1H), 1.05 (dt, J = 9.4, 4.8 Hz, 1H), 1.00-0.90 (m, 1H). | 567.3 |

### Example 266

### Preparation of 5′-(4-aminopiperidin-1l-yl)-3-fluoro-3′,3′-dihydroxy-4′-methyl-[1,1′:2′,1′-triphenyl]-4-carbonitrile

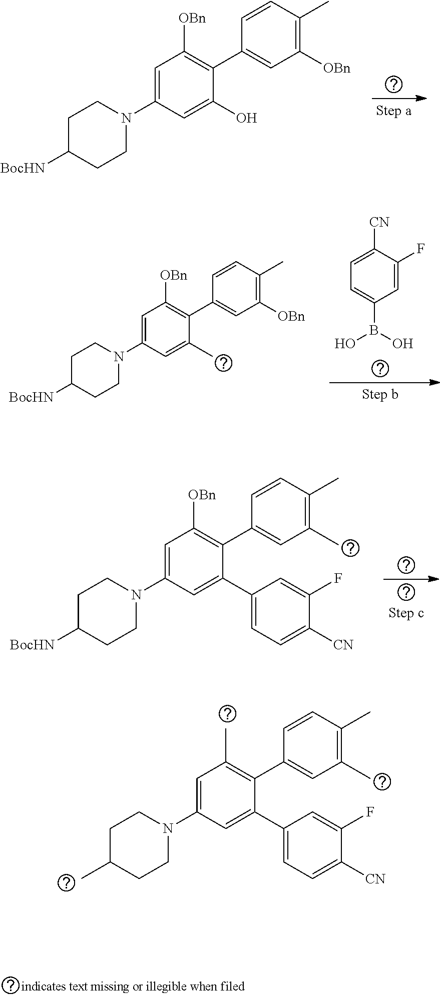 [SMILES: Cc1ccc(-c2c(O)cc(N3CCC(NC(=O)OC(C)(C)C)CC3)cc2OCc2ccccc2)cc1OCc1ccccc1 ; Cc1ccc(-c2c(C)cc(N3CCC(NC(=O)OC(C)(C)C)CC3)cc2OCc2ccccc2)cc1OCc1ccccc1 ; N#Cc1ccc(B(O)O)cc1F ; Cc1ccc(-c2c(OCc3ccccc3)cc(N3CCC(NC(=O)OC(C)(C)C)CC3)cc2-c2ccc(C#N)c(F)c2)cc1C ; Cc1ccc(-c2c(C)cc(N3CCC(C)CC3)cc2-c2ccc(C#N)c(F)c2)cc1C]

### Step a): Preparation of 3′,6-bis(benzyloxy)-4-(4-((tert-butoxycarbonyl)amino)piperidin-1-yl-4′-methyl-[1,1′-biphenyl]-2-yl-trifluoromethane sulfonate

### Tert-butyl

(1-(2,3′-bis(benzyloxy)-6-hydroxy-4′-methyl-[1,1′-biphenyl]-4-yl)piperidin-4-yl-carbamate (90 mg, 0.152 mmol) is dissolved in dichloromethane (5 mL), added with pyridine (24 mg, 0.3 mmol) and trifluoromethanesulfonic anhydride (52 mg, 0.182 mmol) in an ice bath and reacted for 4 h.

When TLC monitors that the raw materials are reacted completely, the reaction solution is quenched with a saturated ammonium chloride aqueous solution (20 mL), and extracted with dichloromethane (15 mL×2); and an organic layer is washed with a saturated sodium bicarbonate aqueous solution (15 mL×2), washed with saturated saline (15 mL×2), dried with anhydrous sodium sulfate, filtered, and concentrated to obtain a crude product 3′,6-bis(benzyloxy)-4-(4-((tert-butoxycarbonyl)amino)piperidin-1-yl)-4′-methyl-[1,1′-biphenyl]-2-yl-trifluoromethane sulfonate with a yield of 92.3%.

ESI-MS m/z=726.3 [M+H]⁺.

### Step b): Preparation of tert-butyl(1-(3,6′-bis(benzyloxy)-4′-cyano-3′-fluoro-4-methyl-[1,1′:2′,1′-triphenyl]-4′-yl)piperidin-4-yl)carbamate

3′,6-bis(benzyloxy)-4-(4-((tert-butoxycarbonyl)amino)piperidin-1-yl)-4′-methyl-[1,1′-biphenyl]-2-yl-trifluoromethane sulfonate (99 mg, 0.14 mmol), (4-cyano-3-fluorophenyl)boronic acid (35 mg, 0.2 mmol), Pd(dppf)Cl₂ (9 mg, 0.014 mmol) and Cs₂CO₃ (91 mg, 0.28 mmol) are dissolved in 3 mL of dioxane: water (4:1) in a microwave tube, continuously blown with nitrogen for 1 min, and reacted in microwaves at 120° C. for 1 h. When LC-MS shows that the reaction is completed, the reaction solution is concentrated under a reduced pressure; and the residue is purified by silica gel chromatography (eluent: petroleum ether/ethyl acetate=5/3) to obtain 4-(3-(2-(1,3-dioxolan-2-yl)ethyl)-5-(4-(methylsulfonyl)piperazin-1-yl)-6-oxypyridine-1(6H)yl)b enzonitrile with a yield of 60.5%.

ESI-MS m/z=698.3 [M+H]⁺.

### Step c): 5′-(4-aminopiperidin-1-yl)-3-fluoro-3′,3′-dihydroxy-4′-methyl-[1,1′:2′,1′-triphenyl]-4-carbonitrile

4-(3-(2-(1,3-dioxolan-2-yl)ethyl)-5-(4-(methylsulfonyl)piperazin-1-yl)-6-oxypyridine-1(6H)yl)b enzonitrile (56 mg, 0.08 mmol) is dissolved in dichloromethane (2 mL), added with a 1M boron tribromide solution (6 mL) at 0° C. and reacted for 30 min; and when LC-MS monitors that the reaction is completed, the residue is concentrated and purified by Prep-HPLC (separation method 1) to obtain 5′-(4-aminopiperidin-1-yl)-3-fluoro-3′,3′-dihydroxy-4′-methyl-[1,1′:2′,1′-triphenyl]-4-carbonitrile dihydrochloride with a yield of 30.2%.

¹H NMR (400 MHz, DMSO-d₆) δppm 9.19 (d, J=8.6.2 Hz, 2H), 8.05 (s, 3H), 7.71 (td, J=8.0, 4.8 Hz, 1H), 7.22 (d, J=10.4 Hz, 1H), 7.01 (d, J=7.8 Hz, 1H), 6.83 (dd, J=7.8, 4.8 Hz, 1H), 6.59 (d, J=12.2 Hz, 3H), 6.27 (d, J=7.4 Hz, 1H), 3.76 (d, J=12.8 Hz, 2H), 3.24 (s, 1H), 2.90 (s, 2H), 2.14-1.78 (m, 5H), 1.67 (s, 2H).

ESI-MS m/z=460.1[M+H]⁺.

### Example 267

### Preparation of 5′-(4-aminopiperidin-1-yl)-3-fluoro-3″-hydroxy-3′,4″-dimethoxy-[1,1′:2′,1″-terphenyl]-4-nitrile trifluoroacetate

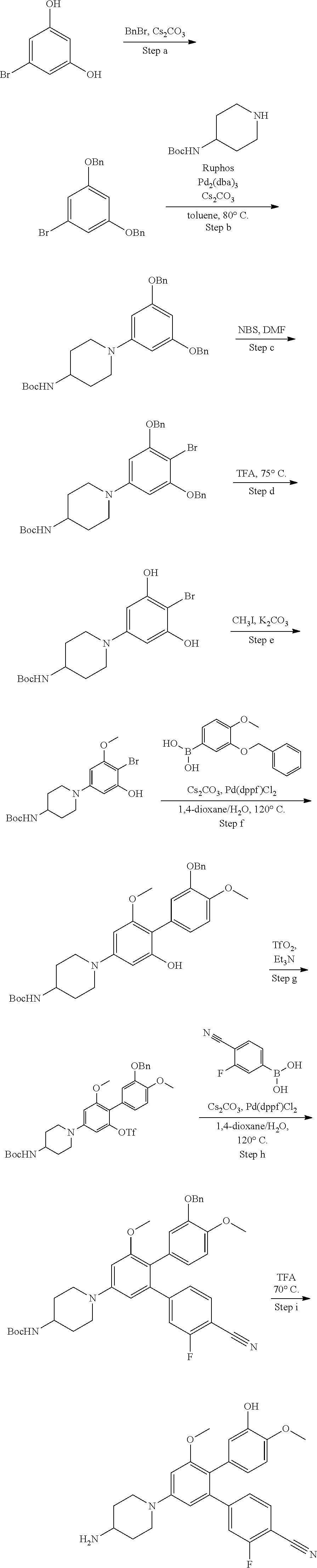

### Step a): Preparation of (((5-bromo-1,3-phenylene)bis(oxy))bis(methylene)diphenyl

Benzyl bromide (4.07 g, 23.82 mmol), 5-bromobenzene-1,3-diol (1.5 g, 7.94 mmol) and cesium carbonate (12.94 g, 39.7 mmol) are sequentially added to a reaction flask containing DMF (20 mL), and the mixture is stirred for 1 h at 70° C. The reaction solution is quenched with water (20 mL), and extracted with ethyl acetate (20 mL×2); and organic phase is washed with saturated saline (15 mL×2), dried with anhydrous sodium sulfate, filtered, and concentrated. The residue is purified by silica gel chromatography (eluent: petroleum ether/ethyl acetate=1: 1) to obtain (((5-bromo-1,3-phenylene)bis(oxy))bis(methylene)diphenyl with a yield of 85.5%.

¹H NMR (400 MHz, DMSO-d₆) δ ppm 7.46-7.29 (m, 10H), 6.83 (d, J=2.2 Hz, 2H), 6.69 (t, J=2.2 Hz, 1H), 5.10 (s, 4H).

ESI-MS m/z=367.1 [M+H]⁺.

### Step b): Preparation of tert-butyl (1-(3,5-bis(benzyloxy)phenyl)piperidin-4-yl)carbamate

(((5-bromo-1,3-phenyl)bis(oxy)bis(methylene)diphenyl (2.8 g, 7.58 mmol), tert-butyl piperidin-4-yl-carbamate (2.28 g, 11.37 mmol), 2-dicyclohexylphosphino-2′,6′-diisopropoxybiphenyl (0.71 g, 1.52 mmol), tris(dibenzylideneacetone)dipalladium (0.44 g, 0.76 mmol) and cesium carbonate (3.70 g, 11.37 mmol) are added to a reaction flask containing toluene (20 mL), and the mixture is stirred for 16 h while heated at 80° C. The reaction solution is filtered to obtain filtrate; the filtrate is then added with silica gel and concentrated; and the residue is purified by silica gel chromatography (eluent: petroleum ether/ethyl acetate=1/1) to obtain tert-butyl (1-(3,5-bis(benzyloxy)phenyl)piperidin-4-yl)carbamate with a yield of 59.4%.

¹H NMR (400 MHz, DMSO-d₆) δ ppm 7.48-7.28 (m, 10H), 6.82 (d, J=7.8 Hz, 1H), 6.15 (d, J=2.2 Hz, 2H), 6.10 (t, J=2.2 Hz, 1H), 5.03 (s, 4H), 3.67-3.56 (m, 2H), 3.31 (d, J=9.6 Hz, 2H), 2.70 (td, J=12.4, 2.6 Hz, 2H), 1.79-1.70 (m, 2H), 1.43 (d, J=3.6 Hz, 1H), 1.39 (s, 9H).

ESI-MS m/z=489.3[M+H]⁺.

### Step c): Preparation of tert-butyl (1-(3,5-bis(benzyloxy)-4-bromophenyl)piperidin-4-yl)carbamate

A DMF solution of NBS (0.58 g, 3.27 mmol) is slowly added dropwise to tert-butyl(3,5-bis(benzyloxy)phenyl)piperidin-4-yl)carbamate (2 g, 4.09 mmol) and DMF (20 mL) at −40° C., and the mixture is stirred at −40° C. for 2 h. The reaction solution is quenched with 20 mL of water, and extracted with ethyl acetate (20 mL×2); organic phase is combined, washed with saturated saline (20 mL×2), dried with anhydrous sodium sulfate, and filtered; and the residue is purified by silica gel chromatography (eluent: petroleum ether/ethyl acetate=1/1) to obtain tert-butyl (1-(3,5-bis(benzyloxy)-4-bromophenyl)piperidin-4-yl)carbamate with a yield of 97.9%.

¹H NMR (400 MHz, DMSO-d6) δ ppm 7.55 (d, J=7.2 Hz, 4H), 7.46 (t, J=7.6 Hz, 4H), 7.39 (d, J=7.2 Hz, 1H), 6.44 (s, 2H), 5.22 (s, 4H), 3.75 (dt, J=13.2, 3.8 Hz, 2H), 2.87-2.75 (m, 2H), 2.56 (p, J=2.0 Hz, 1H), 1.84 (dd, J=13.2, 4.2 Hz, 2H), 1.51 (d, J=3.4 Hz, 2H), 1.46 (s, 9H).

ESI-MS m/z=567.2 [M+H]⁺.

### Step d): Preparation of tert-butyl (1-(4-bromo-3,5-dihydroxyphenyl)piperidin-4-yl)carbamate

Tert-butyl (1-(3,5-bis(benzyloxy)-4-bromophenyl)piperidin-4-yl)carbamate (3 g, 5.29 mmol) is added to a reaction solution containing TFA (100 mL). The mixture is stirred at 75° C. for 16 h; when the reaction is completed, a solvent is removed under a reduced pressure; the mixture is then added with THE (20 mL) and water (20 mL); the pH is adjusted to 8 with a saturated sodium bicarbonate solution; the mixture is cooled to 0° C., and then added with tert-butoxycarbonic anhydride (1.73 g, 7.94 mmol); and the mixture is stirred for 2 h at room temperature. The reaction solution is quenched with water (50 mL), and extracted with ethyl acetate (100 mL×2); organic phase is combined, washed with saturated saline (100 mL×2), dried with anhydrous sodium sulfate, and filtered; the filtrate is concentrated under a reduced pressure; and the residue is purified by silica gel chromatography (eluent: petroleum ether/ethyl acetate=2/1) to obtain tert-butyl (1-(4-bromo-3,5-dihydroxyphenyl)piperidin-4-yl)carbamate with a yield of 39.5%.

¹H NMR (400 MHz, DMSO-d6) δ ppm 9.59 (d, J=2.9 Hz, 1H), 7.32-6.67 (m, 1H), 6.00 (t, J=1.6 Hz, 2H), 3.49-3.40 (m, 2H), 2.68 (td, J=12.6, 2.6 Hz, 2H), 1.99 (s, 1H), 1.75 (dd, J=12.8, 4.0 Hz, 2H), 1.49-1.41 (m, 2H), 1.38 (s, 9H).

ESI-MS m/z=387.1 [M+H]⁺.

### Step e): Preparation of tert-butyl (1-(4-bromo-3-hydroxy-5-methoxyphenyl)piperidin-4-yl)carbamate

Tert-butyl (1-(4-bromo-3,5-dihydroxyphenyl)piperidin-4-yl)carbamate (750 mg, 1.94 mmol), iodomethane (0.17 g, 1.16 mmol) and potassium carbonate (0.80 g, 5.82 mmol) are added to a reaction flask containing DMF (15 mL), and the mixture is stirred for 2 h at room temperature. The reaction solution is quenched with water (20 mL), and extracted with ethyl acetate (20 mL×2); organic phase is combined, washed with saturated saline (15 mL×2), dried with anhydrous sodium sulfate, and filtered; and the residue is purified by silica gel chromatography (eluent: petroleum ether/ethyl acetate=2/1) to obtain tert-butyl (1-(4-bromo-3-hydroxy-5-methoxyphenyl)piperidin-4-yl)carbamate with a yield of 20.6%.

¹H NMR (400 MHz, DMSO-d6) δ ppm 9.76 (s, 1H), 6.85 (d, J=7.8 Hz, 1H), 6.12 (d, J=1.6 Hz, 2H), 3.76 (s, 3H), 3.57 (dt, J=12.8, 3.8 Hz, 2H), 3.34 (s, 1H), 2.77-2.66 (m, 2H), 1.83-1.71 (m, 2H), 1.50-1.42 (m, 2H), 1.39 (s, 9H).

ESI-MS m/z=401.1 [M+H]⁺.

### Step f): Preparation of tert-butyl(1-(3′-(benzyloxy)-2-hydroxy-4′,6-dimethoxy-[1,1′-biphenyl]-4-yl)piperidin-4-yl)carba mate

Tert-butyl (1-(4-bromo-3-hydroxy-5-methoxyphenyl)piperidin-4-yl)carbamate (150 mg, 0.37 mmol), (3-(benzyloxy)-4-methoxyphenyl)boronic acid (0.11 g, 0.43 mmol), Pd(dppf)Cl₂ (0.27 g, 0.37 mmol) and cesium carbonate (0.24 g, 0.74 mmol) are added to a reaction flask containing 1,4-dioxane (2 mL) and water (0.5 mL); and the mixture is stirred at 120° C. in microwaves for 45 min. The reaction solution is then added with silica gel and concentrated; and the residue is purified by silica gel chromatography (eluent: petroleum ether/ethyl acetate=1/1) to obtain tert-butyl(1-(3′-(benzyloxy)-2-hydroxy-4′,6-dimethoxy-[1,1′-biphenyl]-4-yl)piperidin-4-yl)carba mate with a yield of 10.9%.

ESI-MS m/z=535.3 [M+H]+.

### Step g): Preparation of 3′-(benzyloxy)-4-(4-((tert-butoxycarbonyl)amino)piperidin-1-yl)-4′,6-dimethoxy-[1,1′-biphenyl]-2-yl trifluoromethane sulfonate

### Tert-butyl

(1-(3′-(benzyloxy)-2-hydroxy-4′,6-dimethoxy-[1,1′-biphenyl]-4-yl)piperidin-4-yl)carbamate (20 mg, 0.037 mmol) and triethylamine (0.011 g, 0.11 mmol) are added to a reaction flask containing DCM (2 mL); the mixture is stirred and cooled to 0° C., and added with trifluoromethanesulfonic anhydride (0.021 g, 0.074 mmol); and the mixture is stirred at 0° C. for 1 h. TLC detection shows that the reaction is completed. The reaction solution is extracted with water and then concentrated to obtain a crude product 3′-(benzyloxy)-4-(4-((tert-butoxycarbonyl)amino)piperidin-1-yl)-4′,6-dimethoxy-[1,1′-biphenyl]-2-yl trifluoromethane sulfonate.

ESI-MS m/z=667.2 [M+H]⁺.

### Step h): Preparation of tert-butyl(1-(3-(benzyloxy)-4″-cyano-3″-fluoro-4,6′-dimethoxy-[1,1′:2′,1″-tertiphenyl]-4′-yl)piper idin-4-yl)carbamate

3′-(benzyloxy)-4-(4-((tert-butoxycarbonyl)amino)piperidin-1-yl)-4′,6-dimethoxy-[1,1′-biphenyl]-2-yl trifluoromethane sulfonate (30 mg, 0.045 mmol), (4-cyano-3-fluorophenyl)boronic acid (11 mg, 0.068 mmol), Pd(dppf)Cl₂ (3.3 mg, 0.0045 mmol) and cesium carbonate (29 mg, 0.090 mmol) are added to a reaction flask containing 1,4-dioxane (2 mL) and water (0.5 mL); and the mixture is stirred at 120° C. in microwaves for 1 h. The reaction solution is then added with silica gel and concentrated; and the residue is purified by silica gel chromatography (eluent: petroleum ether/ethyl acetate=2/1) to obtain tert-butyl(1-(3-(benzyloxy)-4″-cyano-3″-fluoro-4,6′-dimethoxy-[1,1′:2′,1″-tertiphenyl]-4′-yl)piper idin-4-yl)carbamate with a yield of 31.8%.

ESI-MS m/z=638.3 [M+H]+.

### Step i): Preparation of 5′-(4-aminopiperidin-1-yl)-3-fluoro-3″-hydroxy-3′,4″-dimethoxy-[1,1′:2′,1″-terphenyl]-4-nitrile trifluoroacetate

Tert-butyl(1-(3-(benzyloxy)-4″-cyano-3″-fluoro-4,6′-dimethoxy-[1,1′:2′,1″-tertiphenyl]-4′-yl)pipe ridin-4-yl)carbamate (17 mg, 0.027 mmol) and trifluoroacetic acid (1 mL) are added to a reaction flask, and the mixture is stirred at 70° C. for 3 h. The reaction solution is concentrated, then purified by Prep-HPLC (separation method 2), and freeze-dried to obtain 5′-(4-aminopiperidin-1-yl)-3-fluoro-3″-hydroxy-3′,4″-dimethoxy-[1,1′:2′,1″-terphenyl]-4-nitrile trifluoroacetate with a yield of 8.9%.

¹H NMR (400 MHz, Methanol-d4) δ ppm 7.53 (t, J=7.4 Hz, 1H), 7.11-7.04 (m, 2H), 6.76 (dd, J=5.4, 3.2 Hz, 2H), 6.61-6.53 (m, 2H), 6.40 (ddd, J=8.4, 2.2, 1.0 Hz, 1H), 3.92 (d, J=13.0 Hz, 2H), 3.83 (d, J=1.0 Hz, 3H), 3.77 (s, 3H), 3.30-3.13 (m, 1H), 2.94 (t, J=12.4 Hz, 2H), 2.18-2.08 (m, 2H), 1.80 (qd, J=12.2, 4.2 Hz, 2H).

ESI-MS m/z=448.2 [M+H]⁺.

### Example 268

### Preparation of 4-(4-(4-aminopiperidin-1-yl)-7-(3-hydroxy-4-methylphenyl)furan[3,2-c]pyridin-6-yl)-2-fluorobe nzonitrile hydrochloride

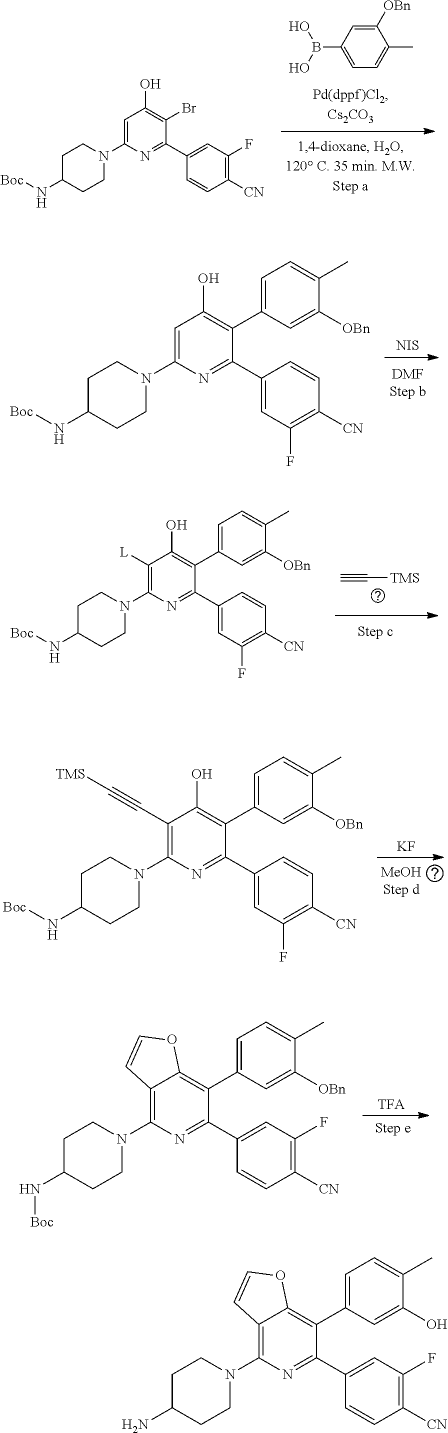

### Step a): Preparation of tert-butyl(1-(5-(3-(benzyloxy)-4-methylphenyl)-6-(4-cyano-3-fluorophenyl)-4-hydroxypyridin-2-yl)piperidin-4-yl)carbamate

Tert-butyl(1-(5-bromo-6-(4-cyano-3-fluorophenyl)-4-hydroxypyridin-2-yl)piperidin-4-yl)carbam ate (2 g, 4.07 mmol), (3-(benzyloxy)-4-methylphenyl)boronic acid (1.2 g, 4.88 mmol), Pd(dppf)Cl₂ (29.7 mg, 0.0407 mmol), and Cs₂CO₃ (2.6 g, 8.14 mmol) are dissolved in 60 mL of dioxane: water (4:1) in a microwave tube, continuously blown with nitrogen for 1 min, and reacted in microwaves at 120° C. for 1 h. When LC-MS shows that the reaction has been completed, the reaction solution is concentrated under a reduced pressure; and the residue is purified by silica gel chromatography (eluent: petroleum ether/ethyl acetate=5/3) to obtain tert-butyl(1-(5-(3-(benzyloxy)-4-methylphenyl)-6-(4-cyano-3-fluorophenyl)-4-hydroxypyridin-2-yl)piperidin-4-yl)carbamate with a yield of 40.5%.

ESI-MS m/z=609.3[M+H]⁺

### Step b): Preparation of tert-butyl(1-(5-(3-(benzyloxy)-4-methylphenyl)-6-(4-cyano-3-fluorophenyl)-4-hydroxy-3-iodopy ridin-2-yl)piperidin-4-yl)carbamate

### Tert-butyl

(1-(5-(3-(benzyloxy)-4-methylphenyl)-6-(4-cyano-3-fluorophenyl)-4-hydroxypyridin-2-yl)piperi din-4-yl)carbamate (403 mg, 0.66 mmol) and NIS (222.73 mg, 0.99 mmol) are dissolved in DMF (10 mL) and reacted for 30 min; upon the complete reaction through detection, the reaction solution is quenched with water, extracted with ethyl acetate, washed with saturated saline, and dried with anhydrous sodium sulfate; and the residue is purified by silica gel chromatography (eluent: petroleum ether/ethyl acetate=5/3) to obtain tert-butyl(1-(5-(3-(benzyloxy)-4-methylphenyl)-6-(4-cyano-3-fluorophenyl)-4-hydroxy-3-iodopy ridin-2-yl)piperidin-4-yl)carbamate with a yield of 90.5%.

ESI-MS m/z=735.2[M+H]⁺

### Step c): Preparation of tert-butyl(1-(5-(3-(benzyloxy)-4-methylphenyl)-6-(4-cyano-3-fluorophenyl)-4-hydroxy-3-((trime thylsilyl)ethynyl)pyridin-2-yl)piperidin-4-yl)carbamate

Tert-butyl(1-(5-(3-(benzyloxy)-4-methylphenyl)-6-(4-cyano-3-fluorophenyl)-4-hydroxy-3-iodop yridin-2-yl)piperidin-4-yl)carbamate (453 mg, 0.62 mmol), cuprous iodide (23.62 mg, 0.12 mmol), and ethyltrimethylsilane (1.2 g, 12.4 mmol) are dissolved in triethylamine (5 mL) and reacted at 25° C. for 18 h under nitrogen protection; when LC-MS shows that the reaction is completed, the reaction solution is concentrated under a reduced pressure; and the residue is purified by silica gel chromatography (eluent: petroleum ether/ethyl acetate=5/4) to obtain tert-butyl(1-(5-(3-(benzyloxy)-4-methylphenyl)-6-(4-cyano-3-fluorophenyl)-4-hydroxy-3-((trime thylsilyl)ethynyl)pyridin-2-yl)piperidin-4-yl)carbamate with a yield of 10.5%.

ESI-MS m/z=705.3[M+H]⁺

### Step d): Preparation of tert-butyl(1-(7-(3-(benzyloxy)-4-methylphenyl)-6-(4-cyano-3-fluorophenyl)furan[3,2-c]pyridin-4-yl)piperidin-4-yl)carbamate

Tert-butyl(1-(5-(3-(benzyloxy)-4-methylphenyl)-6-(4-cyano-3-fluorophenyl)-4-hydroxy-3-((trim ethylsilyl)ethynyl)pyridin-2-yl)piperidin-4-yl)carbamate (100 mg, 0.14 mmol) and potassium fluoride (81.34 mg, 1.40 mmol) are dissolved in methanol (2 mL), warmed to 50° C. and reacted for 4 h; when LC-MS shows that the reaction is completed, the reaction solution is concentrated under a reduced pressure; and the residue is purified by silica gel chromatography (eluent: petroleum ether/ethyl acetate=5/4) to obtain tert-butyl(1-(7-(3-(benzyloxy)-4-methylphenyl)-6-(4-cyano-3-fluorophenyl)furan[3,2-c]pyridin-4-yl)piperidin-4-yl)carbamate with a yield of 20.5%.

ESI-MS m/z=633.3[M+H]⁺

### Step e): Preparation of 4-(4-(4-aminopiperidin-1-yl)-7-(3-hydroxy-4-methylphenyl)furan[3,2-c]pyridin-6-yl)-2-fluorobe nzonitrile

Tert-butyl(1-(7-(3-(benzyloxy)-4-methylphenyl)-6-(4-cyano-3-fluorophenyl)furan[3,2-c]pyridin-4-yl)piperidin-4-yl) carbamate (50 mg, 0.008 mmol) is dissolved in trifluoroacetic acid (6 mL) and reacted at 75° C. for 2 h; when LC-MS monitors that the reaction is completed, the reaction solution is concentrated to obtain a crude product; and the crude product is purified by Prep-HPLC (separation method 1) to obtain 4-(4-(4-aminopiperidin-1-yl)-7-(3-hydroxy-4-methylphenyl)furan[3,2-c]pyridin-6-yl)-2-fluorobe nzonitrile with a yield of 50.2%.

¹H NMR (400 MHz, Methanol-d₄) δppm 7.96 (s, 1H), 7.66 (t, J=7.6 Hz, 1H), 7.52 (d, J=10.4 Hz, 1H), 7.38 (dd, J=8.2, 1.4 Hz, 1H), 7.29 (d, J=17.4 Hz, 1H), 7.12-7.05 (m, 1H), 6.68 (d, J=1.8 Hz, 1H), 6.63 (dd, J=7.6, 1.8 Hz, 1H), 4.64-4.52 (m, 2H), 3.58-3.46 (m, 1H), 2.23 (d, J=2.2 Hz, 4H), 1.97 (s, 1H), 1.86 (q, J=12.4 Hz, 2H), 1.68 (d, J=7.0 Hz, 1H), 1.20 (t, J=7.2 Hz, 1H).

ESI-MS m/z=443.2 [M+H]⁺.

### Example 269

### Preparation of 4-(2-((1R,2S)-2-(4-fluorophenyl)cyclopropyl)amino)methyl)-5-methoxy-6-(4-(methylsulfonyl)pi perazin-1-yl)pyrimidin-4-yl)benzonitrile hydrochloride

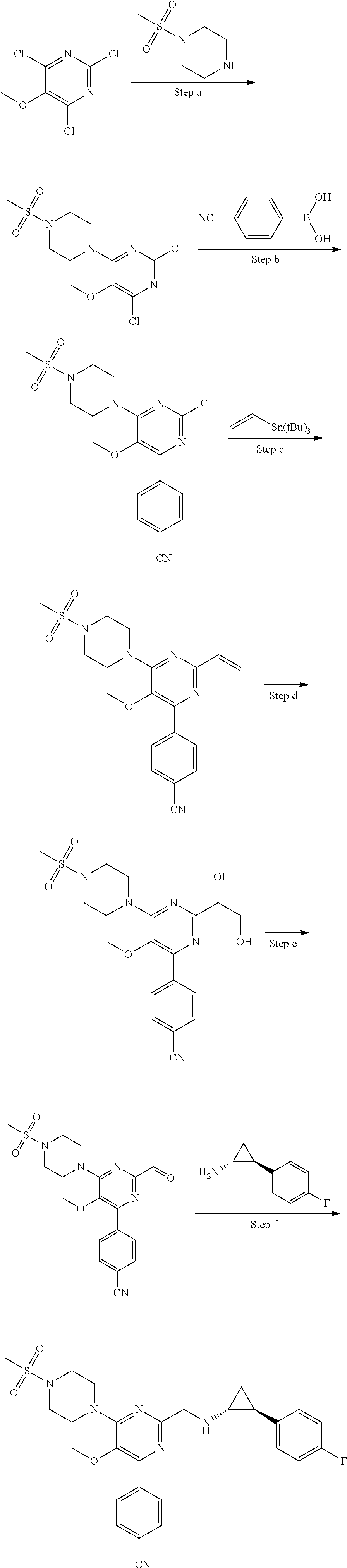

### Step a): Preparation of 2,4-dichloro-5-methoxy-6-(4-(methylsulfonyl)piperazin-1-yl)pyrimidine

2,4,6-trichloro-5-methoxypyrimidine (500 mg, 2.3 mmol) is dissolved in ethanol (10 mL), and added with triethylamine (711.2 mg, 7.0 mmol) and 1-methylsulfonyl piperazine (384.7 mg, 2.3 mmol). The mixture is reacted at 80° C. for 5 h under nitrogen protection; when LC-MS monitors that the raw materials disappear, the reaction solution is cooled to room temperature, poured into ice water and quenched, and extracted with ethyl acetate (20 mL×2); organic phase is combined, washed with saturated saline (15 mL×2), dried with anhydrous sodium sulfate, filtered and concentrated under a reduced pressure; and the residue is purified by silica gel chromatography (eluent: petroleum ether/ethyl acetate=1/1) to obtain 2,4-dichloro-5-methoxy-6-(4-(methylsulfonyl)piperazin-1-yl)pyrimidine with a yield of 68.9%.

ESI-MS m/z=341.2 [M+H]⁺.

### Step b): Preparation of 4-(2-chloro-5-methoxy-6-(4-(methylsulfonyl)piperazin-1-yl)pyrimidin-4-yl)benzonitrile

2,4-dichloro-5-methoxy-6-(4-(methylsulfonyl)piperazin-1-yl)pyrimidine (550 mg, 1.6 mmol), (4-cyanophenyl)boronic acid (237.7 mg, 1.6 mmol), Pd(dppf)Cl₂ (59.2 mg, 0.1 mmol), cesium carbonate (1.6 g, 4.9 mmol), 1,4-dioxane (7 mL) and water (0.7 mL) are sequentially added to a microwave tube, and the mixture is reacted at 60° C. in microwaves for 1.5 h. When LC-MS shows that raw materials are reacted completely, the reaction solution is diluted with water (10 mL), and extracted with ethyl acetate (20 mL×2); organic phase is combined, washed with saturated saline (15 mL×2), dried with anhydrous sodium sulfate, filtered and concentrated under a reduced pressure; and the residue is purified by silica gel chromatography (eluent: petroleum ether/ethyl acetate=1/1) to obtain 4-(2-chloro-5-methoxy-6-(4-(methylsulfonyl)piperazin-1-yl)pyrimidin-4-yl)benzonitrile with a yield of 58.6%.

ESI-MS m/z=407.8 [M+H]⁺.

### Step c): Preparation of 4-(5-methoxy-6-(4-(methylsulfonyl)piperazin-1-yl)-2-vinylpyrimidin-4-yl)benzonitrile

4-(2-chloro-5-methoxy-6-(4-(methylsulfonyl)piperazin-1-yl)pyrimidin-4-yl)benzonitrile (380 mg, 0.9 mmol) is dissolved in DMF (5 mL), added with lithium chloride (395.7 mg, 9.3 mmol), Pd(PPh₃)₄(215.7 mg, 0.2 mmol) and tributylvinyltin (592.0 mg, 1.9 mmol) and reacted at 100° C. for 2 h under nitrogen protection. When LC-MS shows that the reaction is completed, the system is cooled to room temperature, poured into ice water and quenched, and extracted with ethyl acetate (20 mL×2); organic phase is combined, washed with saturated saline (15 mL×2), dried with anhydrous sodium sulfate, filtered and concentrated under a reduced pressure; and the residue is purified by silica gel chromatography (eluent: petroleum ether/ethyl acetate=1/1) to obtain 4-(5-methoxy-6-(4-(methylsulfonyl)piperazin-1-yl)-2-vinylpyrimidin-4-yl)benzonitrile with a yield of 36.7%.

ESI-MS m/z=399.5 [M+H]⁺.

### Step d): Preparation of 4-(2,2-dihydroxyethyl)-5-methoxy-6-(4-(methylsulfonyl)piperazin-1-yl)pyrimidin-4-yl)benzonitr ile

4-(5-methoxy-6-(4-(methylsulfonyl)piperazin-1-yl)-2-vinylpyrimidin-4-yl)benzonitrile (136.7 mg, 0.3 mmol) is dissolved in acetonitrile (2 mL), added with potassium osmium dihydrate (12.6 mg, 34.2 mol) and N-methylmorpholine oxide (82.2 mg, 0.7 mmol), and reacted for 2 h at room temperature. When LC-MS shows that the reaction has been completed, the reaction solution is extracted with ethyl acetate to obtain a product; an organic phase is dried with anhydrous sodium sulfate, and concentrated under vacuum; and the residue is purified by silica gel chromatography (eluent: dichloromethane/ethyl acetate=1/1) to obtain 4-(2,2-dihydroxyethyl)-5-methoxy-6-(4-(methylsulfonyl)piperazin-1-yl)pyrimidin-4-yl)benzonitr ile with a yield of 74.2%.

ESI-MS m/z=434.2 [M+H]⁺.

### Step e): Preparation of 4-(2-formyl-5-methoxy-6-(4-(methylsulfonyl)piperazin-1-yl)pyrimidin-4-yl)benzonitrile

4-(2,2-dihydroxyethyl)-5-methoxy-6-(4-(methylsulfonyl)piperazin-1-yl)pyrimidin-4-yl)benzonitr ile (110 mg, 254.1 mol) is dissolved in acetonitrile (1 mL) and water (0.1 mL), added with sodium periodate (163 mg, 761.9 mol), and reacted overnight at room temperature; when LC-MS shows that the reaction has been completed, the reaction solution is extracted with ethyl acetate (20 mL×2) to obtain a product; organic phase is combined, washed with saturated saline (15 mL×2), dried with anhydrous sodium sulfate, filtered and concentrated under a reduced pressure; and the residue is purified by silica gel chromatography (eluent: dichloromethane/ethyl acetate=1:1) to obtain 4-(2-formyl-5-methoxy-6-(4-(methylsulfonyl)piperazin-1-yl)pyrimidin-4-yl)benzonitrile with a yield of 71.2%.

ESI-MS m/z=402.1 [M+H]⁺.

### Step f): Preparation of 4-(2-((1R,2S)-2-(4-fluorophenyl)cyclopropyl)amino)methyl)-5-methoxy-6-(4-(methylsulfonyl)pi perazin-1-yl)pyrimidin-4-yl)benzonitrile

4-(2-Formyl-5-methoxy-6-(4-(methylsulfonyl)piperazin-1-yl)pyrimidin-4-yl)benzonitrile (72.5 mg, 180.8 mol), and (1R,2S)-2-(4-fluorophenyl)cyclopropylamine; 2-(4-fluorophenyl)cyclopropylamine (34 mg, 180.8 mol) are dissolved in 1,2-dichloroethane (2 mL), then added into methanol (0.4 mL) and acetic acid (0.1 mL), reacted at room temperature for 1 h, then added with sodium cyanoborohydride (56.8 mg, 0.9 mmol), and reacted overnight at room temperature; when LC-MS shows that the reaction has been completed, the reaction solution is quenched with water, and extracted with ethyl acetate (10 mL×2) to obtain a product; organic phase is combined, washed with saturated saline (10 mL×2), dried with anhydrous sodium sulfate, filtered and concentrated under a reduced pressure; and the residue is subjected to Prep-HPLC (separation method 1) to obtain 4-(2-((1R,2S)-2-(4-fluorophenyl)cyclopropyl)amino)methyl)-5-methoxy-6-(4-(methylsulfonyl)pi perazin-1-yl)pyrimidin-4-yl)benzonitrile with a yield of 28.8%.

¹H NMR (400 MHz, Methanol-d₄) δ 8.65-8.54 (m, 2H), 7.88-7.76 (m, 2H), 7.25-7.14 (m, 2H), 7.09-6.98 (m, 2H), 4.64 (s, 2H), 4.00 (t, J=5.0 Hz, 4H), 3.80 (s, 3H), 3.40 (t, J=5.0 Hz, 4H), 3.15 (dt, J=8.0, 4.0 Hz, 1H), 2.88 (s, 3H), 2.68-2.56 (m, 1H), 1.71-1.59 (m, 1H), 1.48-1.39 (m, 1H).

ESI-MS m/z=537.2 [M+H]⁺.

### Example 270

### Preparation of 4-(4-(4-aminopiperidin-1-yl)-7-(3-fluoro-4-methoxyphenyl)-1H-imidazolo[4,5-c]pyridin-6-yl)-2-fluorobenzonitrile hydrochloride

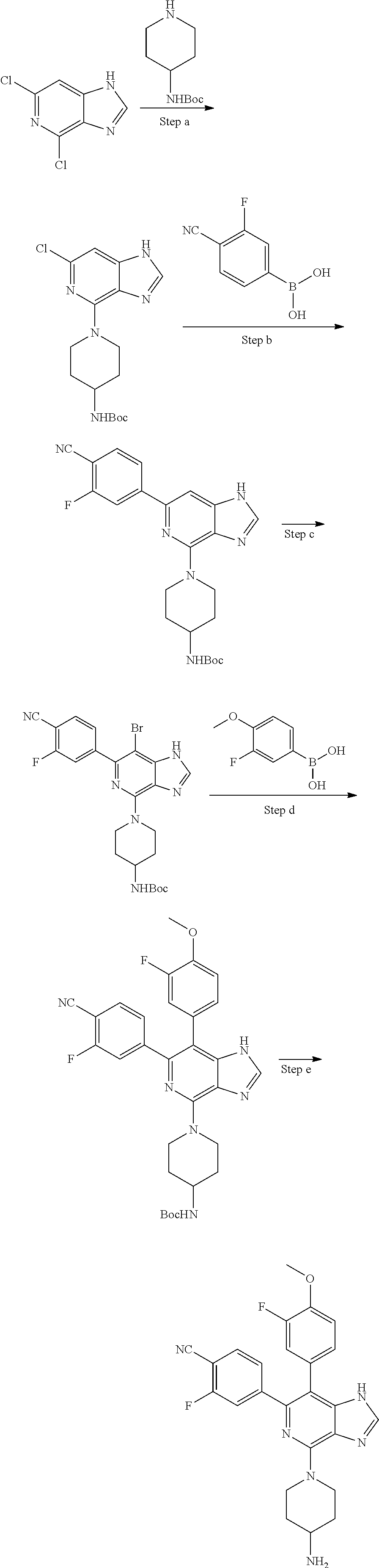

### Step a): Preparation of tert-butyl (1-(6-chloro-1H-imidazolo[4,5-c]pyridin-4-yl)piperidin-4-yl)carbamate

4,6-dichloro-1H-imidazolo[4,5-c]pyridine (200 mg, 1.1 mmol) and tert-butyl piperidin-4-yl-carbamate (213.1 mg, 1.1 mol) are dissolved in isopropanol (30 mL), added with triethylamine (323 mg, 3.2 mmol), warmed to 120° C. and reacted for 16 h. When LC-MS monitors that raw materials are reacted completely, the reaction solution is cooled to room temperature, then quenched with water (20 mL), and extracted with ethyl acetate (20 mL×2); organic phase is combined, washed with saturated saline (15 mL×2), dried with anhydrous sodium sulfate, filtered and concentrated under a reduced pressure; and the residue is purified by silica gel chromatography (eluent: petroleum ether/ethyl acetate=5/2) to obtain tert-butyl (1-(6-chloro-1H-imidazol[4,5-c]pyridin-4-yl)piperidin-4-yl)carbamate with a yield of 32.1%.

¹H NMR (400 MHz, DMSO-d₆) δ 12.66 (s, 1H), 8.09 (s, 1H), 6.83 (d, J=8.0 Hz, 1H), 6.79 (s, 1H), 5.12 (d, J=13.2 Hz, 2H), 3.55 (s, 1H), 3.15-3.03 (m, 2H), 1.86-1.73 (m, 2H), 1.39 (s, 9H), 1.37-1.31 (m, 2H).

ESI-MS m/z=352.2 [M+H]⁺.

### Step b): Preparation of tert-butyl (1-(6-(4-cyano-3-fluorophenyl)-1H-imidazolo[4,5-c]pyridin-4-yl)piperidin-4-yl-carbamate

Tert-butyl(1-(6-chloro-1H-imidazolo[4,5-c]pyridin-4-yl)piperidin-4-yl)carbamate (120 mg, 340 mol), (4-cyano-3-fluorophenyl)boronic acid (52.3 mg, 340 mol), Pd(dppf)Cl₂ (24.7 mg, 34 mol), cesium carbonate (333.4 mg, 1 mmol), and 1,4-dioxane: water=10:1 (3 mL) are sequentially added into a microwave tube, continuously blown with nitrogen for 1 min, and reacted in microwaves at 110° C. for 2 h. When LC-MS shows that raw materials are reacted completely, the system is cooled to room temperature, then quenched with water (20 mL), and extracted with ethyl acetate (10 mL×2); organic phase is combined, washed with saturated saline (15 mL×2), dried with anhydrous sodium sulfate, filtered and concentrated under a reduced pressure; and the residue is purified by silica gel chromatography (eluent: petroleum ether/ethyl acetate=5/2) to obtain tert-butyl (1-(6-(4-cyano-3-fluorophenyl)-1Himidazole[4,5-c]pyridin-4-yl)piperidin-4-ylcarbamate with a yield of 67.2%.

ESI-MS m/z=437.2 [M+H]⁺.

### Step c): Preparation of tert-butyl (1-(7-bromo-6-(4-cyano-3-fluorophenyl)-1H-imidazolo[4,5-c]pyridin-4-yl)piperidin-4-yl-carbam ate

Tert-butyl(1-(6-(4-cyano-3-fluorophenyl)-1H imidazolo[4,5-c]pyridin-4-yl)piperidin-4-yl-carbamate (100 mg, 229.1 mol) and NBS (27 mg, 229.1 mol) are dissolved in DMF, added dropwise to the system, and reacted for 1 h at room temperature. When LC-MS shows that the reaction is completed, the system is quenched with ice water, and extracted with ethyl acetate (10 mL×2); organic phase is combined, washed with saturated saline (15 mL×2), dried with anhydrous sodium sulfate, filtered and concentrated under a reduced pressure; and the residue is purified by silica gel chromatography (eluent: petroleum ether/ethyl acetate=5/2) to obtain tert-butyl (1-(7-bromo-6-(4-cyano-3-fluorophenyl)-1H-imidazolo[4,5-c]pyridin-4-yl)piperidin-4-yl-carbam ate with a yield of 67.8%.

ESI-MS m/z=515.1 [M+H]⁺.

### Step d): Preparation of tert-butyl (1-(6-(4-cyano-3-fluorophenyl)-7-(3-fluoro-4-methoxyphenyl)-1H-imidazolo[4,5-c]pyridin-4-yl) piperidin-4-yl-carbamate

### Tert-butyl

(1-(7-bromo-6-(4-cyano-3-fluorophenyl)-1H-imidazolo[4,5-c]pyridin-4-yl)piperidin-4-yl-carbam ate (80 mg, 155.2 mol), (3-cyano-4-fluorophenyl)boronic acid (26.4 mg, 155.2 mol), Pd(dppf)Cl₂ (11.3 mg, 15.5 mol), cesium carbonate (151.7 mg, 465.7 mol), and 1,4-dioxane: water=10:1 (2 mL) are sequentially added into a microwave tube, continuously blown with nitrogen for 1 min, and reacted in microwaves at 110° C. for 2 h. When LC-MS shows that raw materials are reacted completely, the system is cooled to room temperature, then quenched with water (10 mL), and extracted with ethyl acetate (10 mL×2); organic phase is combined, washed with saturated saline (15 mL×2), dried with anhydrous sodium sulfate, filtered and concentrated under a reduced pressure; and the residue is purified by silica gel chromatography (eluent: petroleum ether/ethyl acetate=5/2) to obtain tert-butyl (1-(6-(4-cyano-3-fluorophenyl)-7-(3-fluoro-4-methoxyphenyl)-1H-imidazolo[4,5-c]pyridin-4-yl) piperidin-4-ylcarbamate with a yield of 69.00.

ESI-MS m/z=561.2 [M+H]⁺.

### Step e): Preparation of 4-(4-(4-aminopiperidin-1-yl)-7-(3-fluoro-4-methoxyphenyl)-1H-imidazolo[4,5-c]pyridin-6-yl)-2-fluorobenzonitrile hydrochloride

Tert-butyl(1-(6-(4-cyano-3-fluorophenyl)-7-(3-fluoro-4-methoxyphenyl)-1H-imidazolo[4,5-c]pyr idin-4-yl)piperidin-4-yl-carbamate (60 mg, 0.11 mmol) is dissolved in 4M hydrochloric acid (ethyl acetate solution, 2 mL) and reacted for 1 h at room temperature. When LC-MS shows that the reaction is completed, the reaction solution is concentrated under a reduced pressure, and subjected to Prep-HPLC (separation method 1) to obtain 4-(4-(4-aminopiperidin-1l-yl)-7-(3-fluoro-4-methoxyphenyl)-1H-imidazolo[4,5-c]pyridin-6-yl)-2-fluorobenzonitrile hydrochloride with a yield of 72.4%.

¹H NMR (400 MHz, Methanol-d₄) δ 8.40 (d, J=13.8 Hz, 1H), 7.75 (q, J=8.4 Hz, 1H), 7.56 (t, J=10.0 Hz, 1H), 7.37 (t, J=8.2 Hz, 1H), 7.20-7.07 (m, 2H), 6.97 (d, J=8.6 Hz, 1H), 5.22 (d, J=13.8 Hz, 2H), 3.95-3.76 (s, 3H), 3.65-3.54 (m, 2H), 3.48 (p, J=1.6 Hz, 1H), 2.26 (s, 2H), 2.02-1.84 (m, 2H).

ESI-MS m/z=461.2 [M+H]+.

### Examples 271-272

Compounds are prepared according to the synthesis method of Example 270 (hydrochloride, separation method 1; free amine, separation method 4), and their structures and characterization data are as follows:

| | | | | MS |
|---|---|---|---|---|
| Examples | Chinese name | Structural formula | 1H NMR | (M + H)⁺ |
| 271 | 4-(4-(4-aminopiperidin- 1-yl)-7-(3-hydroxy-4- methoxyphenyl)-1H- imidazolo[4,5-c]pyridin-6- yl)-2-fluorobenzonitrile hydrochloride | 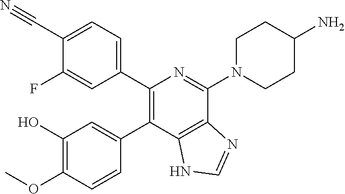 [SMILES: COc1ccc(-c2c(-c3ccc(C#N)c(F)c3)nc(N3CCC(N)CC3)c3nc[nH]c23)cc1O] | ¹H NMR (400 MHz, DMSO-d₆) δ 8.45 (s, 1H), 8.39 (s, 3H), 7.90 (t, J = 7.6 Hz, 1H), 7.60 (d, J = 10.8 Hz, 1H), 7.37 (dd, J = 8.1, 1.6 Hz, 1H), 7.01 (d, J = 8.4 Hz, 1H), 6.74 (d, J = 2.2 Hz, 1H), 6.66 (dd, J = 8.2, 2.2 Hz, 1H), 5.23 (d, J = 13.6 Hz, 2H), 3.85 (s, 3H), 3.39 (m, 3H), 2.15 (d, J = 12.4 Hz, 2H), 1.77 (t, J = 12.6 Hz, 2H). | 459.2 |
| 272 | 4-(4-(4-aminopiperidin- 1-yl)-7-(5-fluoro-3- methylbenzo[d]isoxazol-6- yl)-1H-imidazolo[4,5- c]pyridin-6-yl)-2- fluorobenzonitrile | 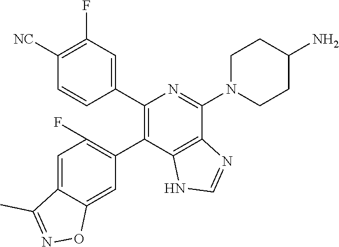 [SMILES: Cc1noc2cc(-c3c(-c4ccc(C#N)c(F)c4)nc(N4CCC(N)CC4)c4nc[nH]c34)c(F)cc12] | ¹H NMR (400 MHz, Methanol-d₄) δ 7.95 (s, 1H), 7.56-7.45 (m, 2H), 7.41 (dd, J = 8.2, 6.7 Hz, 1H), 7.32 (dd, J = 10.8, 1.6 Hz, 1H), 7.16 (dd, J = 8.2, 1.5 Hz, 1H), 5.20 (t, J = 13.2 Hz, 2H), 3.15-3.02 (m, 2H), 3.01-2.93 (m, 1H), 2.49 (s, 3H), 1.90 (d, J = 12.8 Hz, 2H), 1.45 (dq, J = 17.8, 11.7, 8.8 Hz, 2H). | 486.2 |

### Example 273

### Preparation of (6-(4-aminopiperidin-1-yl)-2-(4-cyano-3-fluorophenyl)-3-(3-hydroxy-4-methoxyphenyl)pyridine-4-carbonitrile)

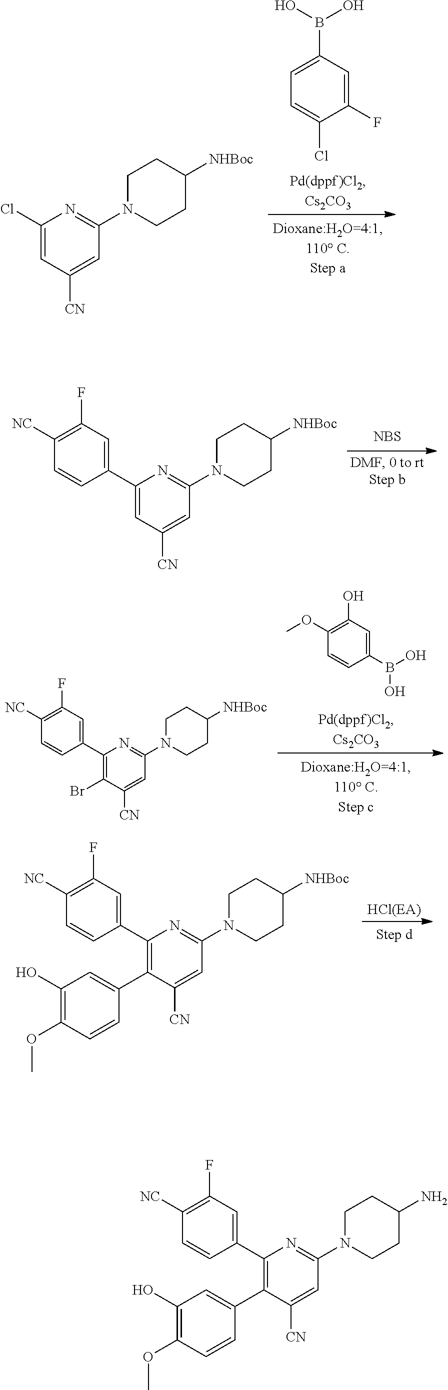

### Step a): Preparation of (tert-butyl N-(1-(4-cyano-6-(4-cyano-3-fluorophenyl)pyridin-2-yl)piperidin-4-yl)carbamate)

(Tert-butyl N-(1-(6-chloro-4-cyanopyridin-2-yl)piperidin-4-yl)carbamate (400 mg, 1.1 mmol), ((4-cyano-3-fluorophenyl)boronic acid) (381.2 mg, 2.4 mmol), cesium carbonate (845.8 mg, 2.6 mmol), and Pd(dppf)Cl₂ (86.4 mg, 0.1 mmol) are added to 12.5 mL of Dioxane/H₂O (4:1), subjected to nitrogen displacement, and reacted in microwaves at 115° C. for 1.5 h; and when LC-MS shows that the raw materials disappear, the solution is concentrated, added with silica gel to be mixed, and purified by a normal-phase column to obtain the product tert-butyl N-(1-(4-cyano-6-(4-cyano-3-fluorophenyl)pyridin-2-yl)piperidin-4-yl)carbamate with a yield of 72.3%.

ESI-MS m/z=422.4 [M+H]+.

### Step b): Preparation of (tert-butyl N-(1-(5-bromo-4-cyano-6-(4-cyano-3-fluorophenyl)pyridin-2-yl)piperidin-4-yl)carbamate)

(Tert-butyl N-(1-(4-cyano-6-(4-cyano-3-fluorophenyl)pyridin-2-yl)piperidin-4-yl)carbamate) (350.0 mg, 0.8 mmol) is dissolved in DMF (4 mL), cooled in an ice bath, added with NBS (162.5 mg, 0.9 mmol), and reacted in the ice bath for 1 h; and when LC-MS shows that the raw materials disappear, the reaction solution is poured into a saturated sodium sulfite solution and extracted with ethyl acetate, and the organic phase is washed with sodium sulfite and saline. The solution is concentrated, added with silica gel to be mixed, and purified by a normal-phase column to obtain the product (tert-butyl N-(1-(5-bromo-4-cyano-6-(4-cyano-3-fluorophenyl)pyridin-2-yl)piperidin-4-yl)carbamate) with a yield of 74.6%.

ESI-MS m/z=500.6 [M+H]⁺.

### Step c): Preparation of (tert-butyl N-(1-(3-cyano-4-(4-cyano-3-fluorophenyl)-5-(3-hydroxy-4-methoxyphenyl)-6-methylpyridin-2-yl)piperidin-4-yl)carbamate)

(Tert-butyl N-(1-(5-bromo-4-cyano-6-(4-cyano-3-fluorophenyl)pyridin-2-yl)piperidin-4-yl)carbamate) (80.0 mg, 0.1 mmol), (2-methoxy-5-(tetramethyl-1,3,2-dioxybenzaldehyde-2-yl)phenol) (60.2 mg, 0.2 mmol), cesium carbonate (104.2 mg, 0.3 mmol) and Pd(dppf)Cl₂ (11.7 mg, 0.1 mmol) are added to 5 mL of Dioxane/H₂O (4:1), subjected to nitrogen protection, and then reacted in microwaves at 115° C. for 1.5 h; and when LC-MS monitors that the raw materials disappear, the reaction solution is concentrated to remove a solvent. The system is purified by a silica gel column (EA:PE=1:1) to obtain the product tert-butyl N-(1-(3-cyano-4-(4-cyano-3-fluorophenyl)-5-(3-hydroxy-4-methoxyphenyl)-6-methylpyridin-2-yl)piperidin-4-yl)carbamate with a yield of 45.6%.

ESI-MS m/z=544.3 [M+H]+.

### Step d): Preparation of (6-(4-aminopiperidin-1-yl)-2-(4-cyano-3-fluorophenyl)-3-(3-hydroxy-4-methoxyphenyl)pyridin-4-carbonitrile)

(Tert-butyl N-(1-(3-cyano-4-(4-cyano-3-fluorophenyl)-5-(3-hydroxy-4-methoxyphenyl)-6-methylpyridin-2-yl)piperidin-4-yl) carbamate) (40.0 mg, 0.1 mmol) is added with a 4N hydrochloric acid (ethyl acetate solution, 8 mL), and reacted at room temperature for 0.5 h; and when LC-MS monitors that the raw materials disappear, the solution is concentrated and purified by Prep-HPLC (separation method 4) to obtain (6-(4-aminopiperidin-1-yl)-2-(4-cyano-3-fluorophenyl)-3-(3-hydroxy-4-methoxyphenyl)pyridin-4-carbonitrile) with a yield of 71.5%.

¹H NMR (400 MHz, DMSO-d₆) δ 7.81 (dd, J=8.2, 6.9 Hz, 1H), 7.50 (s, 1H), 7.36 (dd, J=10.8, 1.4 Hz, 1H), 7.22 (dd, J=8.2, 1.6 Hz, 1H), 6.90 (d, J=8.2 Hz, 1H), 6.71-6.44 (m, 2H), 4.29 (dd, J=13.2, 4.1 Hz, 2H), 3.77 (s, 3H), 3.03 (ddd, J=13.8, 11.3, 2.7 Hz, 2H), 2.85 (ddt, J=14.2, 9.6, 4.0 Hz, 1H), 1.78 (dt, J=11.4, 3.4 Hz, 2H), 1.33-1.11 (m, 2H).

ESI-MS m/z=444.2[M+H]⁺.

### Example 274

### Preparation of (6-(4-aminopiperidin-1-yl)-3-(4-cyano-3-fluorophenyl)-2-(3-hydroxy-4-methoxyphenyl)pyridin-4-carbonitrile)

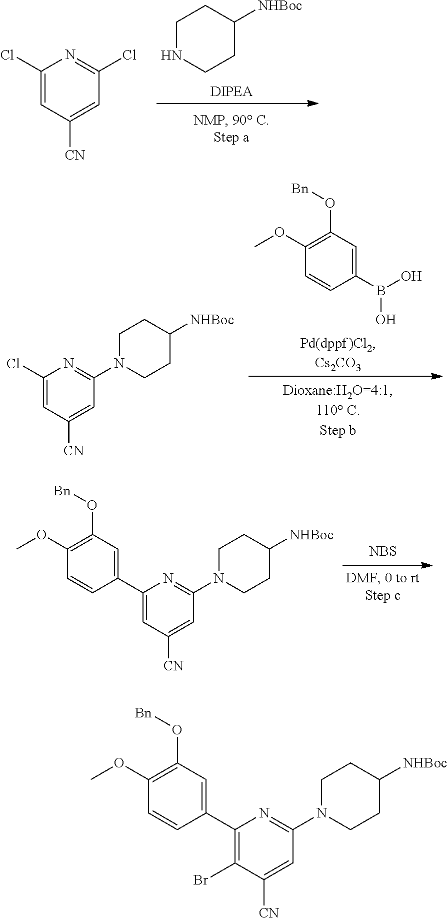

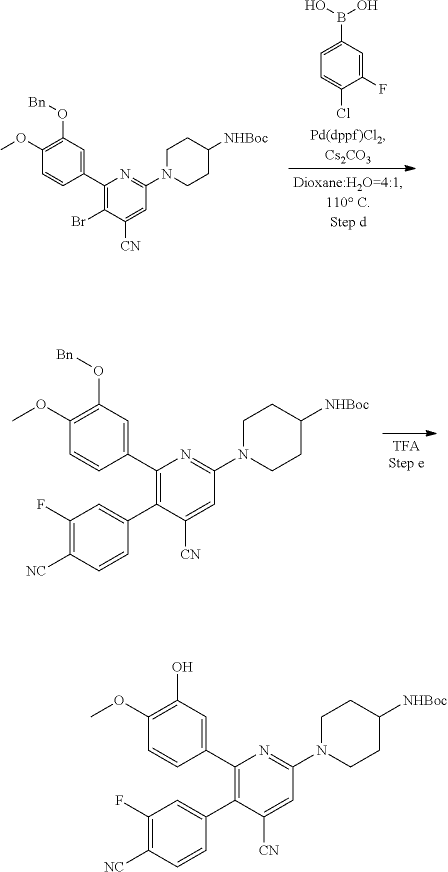 [SMILES: COc1ccc(-c2nc(N3CCC(NC(=O)OC(C)(C)C)CC3)cc(C#N)c2Br)cc1OCc1ccccc1 ; OB(O)c1ccc(Cl)c(F)c1 ; COc1ccc(-c2nc(N3CCC(NC(=O)OC(C)(C)C)CC3)cc(C#N)c2-c2ccc(C#N)c(F)c2)cc1OCc1ccccc1 ; COc1ccc(-c2nc(N3CCC(NC(=O)OC(C)(C)C)CC3)cc(C#N)c2-c2ccc(C#N)c(F)c2)cc1O]

### Step a): Preparation of (tert-butyl(N-(1-(6-chloro-4-ethynylpyridin-2-yl)piperidin-4-yl-carbamate)

(2,6-Dichloro-4-ethynylpyridine) (1 g, 5.8 mmol) is dissolved in NMP (10 mL), added with (tert-butyl(N-(piperidin-4-yl)carbamate) (2.33 g, 11.6 mmol), and reacted at 60° C. overnight; and when the LC-MS shows that the raw materials disappear, the system is added to water, and extracted with ethyl acetate to obtain a crude product (tert-butyl(N-(1-(6-chloro-4-ethynylpyridin-2-yl)piperidin-4-yl)carbamate) with a yield of 86%.

ESI-MS m/z=337.3 [M+H]+.

### Step b): Preparation of (tert-butyl N-(1-(6-(3-(benzyloxy)-4-methoxyphenyl)-4-cyanopyridin-2-yl)piperidin-4-yl)carbamate)

(Tert-butyl N-(1-(6-chloro-4-ethynylpyridin-2-yl)piperidin-4-yl)carbamate) (300.0 mg, 0.8 mmol), ((3-(benzyloxy)-4-methoxyphenyl)boronic acid) (367.5 mg, 1.4 mmol), cesium carbonate (637.9 mg, 1.9 mmol), and Pd(dppf)Cl₂ (65.1 mg, 0.1 mmol) are added to 5 mL of Dioxane/H₂O=4:1, subjected to nitrogen displacement, and reacted in microwaves at 115° C. for 1.5 h; and when LC-MS shows that the raw materials disappear, the solution is concentrated, added with silica gel to be mixed, and purified by a normal-phase column to obtain the product (tert-butyl N-(1-(6-(3-(benzyloxy)-4-methoxyphenyl)-4-cyanopyridin-2-yl)piperidin-4-yl)carbamate) with a yield of 84.0%.

ESI-MS m/z=515.2 [M+H]+.

### Step c): Preparation of (tert-butyl N-(1-(6-(3-(benzyloxy)-4-methoxyphenyl)-5-bromo-4-cyanopyridin-2-yl)piperidin-4-yl)carbamate)

(Tert-butyl N-(1-(6-(3-(benzyloxy)-4-methoxyphenyl)-4-cyanopyridin-2-yl)piperidin-4-yl)carbamate) (550.0 mg, 1.0 mmol) is dissolved in DMF (6 mL), cooled in an ice bath, added with NBS (209.4 mg, 1.1 mmol), and reacted in the ice bath for 1 h; and when LC-MS shows that the raw materials disappear, the reaction solution is poured into a saturated sodium sulfite solution and extracted with ethyl acetate, and the organic phase is washed with sodium sulfite and saline. The solution is concentrated to remove a solvent, added with silica gel to be mixed, and purified by a silica gel column to obtain the product (tert-butyl N-(1-(6-(3-(benzyloxy)-4-methoxyphenyl)-5-bromo-4-cyanopyridin-2-yl)piperidin-4-yl)carbamate) with a yield of 89.7%.

ESI-MS m/z=593.7 [M+H]+.

### Step d): Preparation of (tert-butyl N-(1-(6-(3-(benzyloxy)-4-methoxyphenyl)-4-cyano-5-(4-cyano-3-fluorophenyl)pyridin-2-yl)pipe ridin-4-yl)carbamate)

(Tert-butyl N-(1-(6-(3-(benzyloxy)-4-methoxyphenyl)-5-bromo-4-cyanopyridin-2-yl)piperidin-4-ylcarbamat e) (100.0 mg, 0.1 mmol), ((4-cyano-3-fluorophenyl)boronic acid) (42.0 mg, 0.2 mmol), cesium carbonate (110.7 mg, 0.3 mmol) and Pd(dppf)Cl₂ (12.4 mg, 0.1 mmol) are added to 2 mL of Dioxane/H₂O (4:1), subjected to nitrogen protection, and then reacted in microwaves at 115° C. for 1.5 h; and when LC-MS monitors that the raw materials disappear, the solution is concentrated. The system is purified by a silica gel column (EA:PE=1:1) to obtain the product (tert-butyl N-(1-(6-(3-(benzyloxy)-4-methoxyphenyl)-4-cyano-5-(4-cyano-3-fluorophenyl)pyridin-2-yl)pipe ridin-4-yl)carbamate) with a yield of 71.2%.

ESI-MS m/z=634.5 [M+H]+.

### Step e): Preparation of (6-(4-aminopiperidin-1-yl)-3-(4-cyano-3-fluorophenyl)-2-(3-hydroxy-4-methoxyphenyl)pyridin-4-carbonitrile)

(Tert-butyl N-(1-(6-(3-(benzyloxy)-4-methoxyphenyl)-4-cyano-5-(4-cyano-3-fluorophenyl)pyridin-2-yl)pipe ridin-4-yl)carbamate) (40.0 mg, 0.1 mmol) is added with TFA (2 mL), and reacted at room temperature for 0.5 h; and when LC-MS monitors that the raw materials disappear, the solution is concentrated and purified by Prep-HPLC (separation method 4) to obtain (6-(4-aminopiperidin-1-yl)-3-(4-cyano-3-fluorophenyl)-2-(3-hydroxy-4-methoxyphenyl)pyridin-4-carbonitrile) with a yield of 94%.

¹H NMR (400 MHz, DMSO-d₆) δ 9.03 (s, 1H), 7.90 (dd, J=8.2, 7.0 Hz, 1H), 7.52 (dd, J=10.4, 1.5 Hz, 1H), 7.41 (s, 1H), 7.21 (dd, J=8.0, 1.6 Hz, 1H), 6.83-6.70 (m, 2H), 6.56 (dd, J=8.4, 2.2 Hz, 1H), 4.33 (d, J=13.6 Hz, 2H), 3.72 (s, 3H), 3.05 (ddd, J=13.8, 11.3, 2.8 Hz, 2H), 2.87 (tt, J=9.8, 4.0 Hz, 1H), 1.79 (dd, J=13.2, 3.7 Hz, 2H), 1.21 (q, J=13.2, 11.3 Hz, 2H).

ESI-MS m/z=444.2[M+H]+.

### Example 275

Compound is prepared according to the synthesis method of Example 274 (separation method 4), and its structure and characterization data are as follows:

### 6-(4-aminopiperidin-1-yl)-2-(4-cyano-3-fluorophenyl)-3-(5-fluoro-3-methylbenzo[d]isoxazol-6-yl)isonionitrile

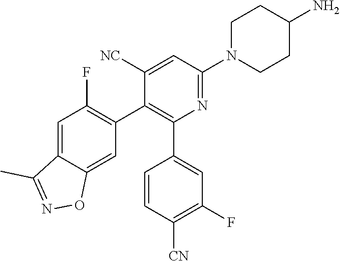 [SMILES: Cc1noc2cc(-c3c(C#N)cc(N4CCC(N)CC4)nc3-c3ccc(C#N)c(F)c3)c(F)cc12]

¹H NMR (400 MHz, DMSO-d₆) δ 7.88 (d, J=5.6 Hz, 1H), 7.84-7.73 (m, 2H), 7.66 (s, 1H), 7.45 (dd, J=10.4, 1.5 Hz, 1H), 7.17 (dd, J=8.2, 1.5 Hz, 1H), 4.44-4.26 (m, 2H), 3.18-3.01 (m, 2H), 2.90 (ddt, J=13.8, 9.4, 3.9 Hz, 1H), 2.55 (s, 3H), 1.81 (d, J=12.8 Hz, 2H), 1.36-1.15 (m, 2H).

ESI-MS m/z=471.2[M+H]⁺.

### Example 276

### Preparation of (2-(4-aminopiperidin-1-yl)-4-(4-cyano-3-fluorophenyl)-5-(3-hydroxy-4-methoxyphenyl)-6-meth ylpyridine-3-carbonitrile)

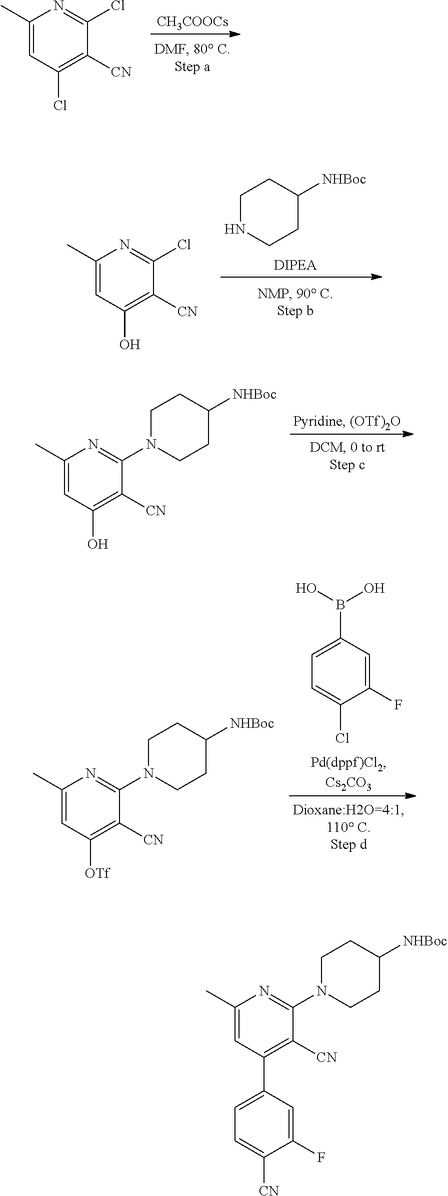

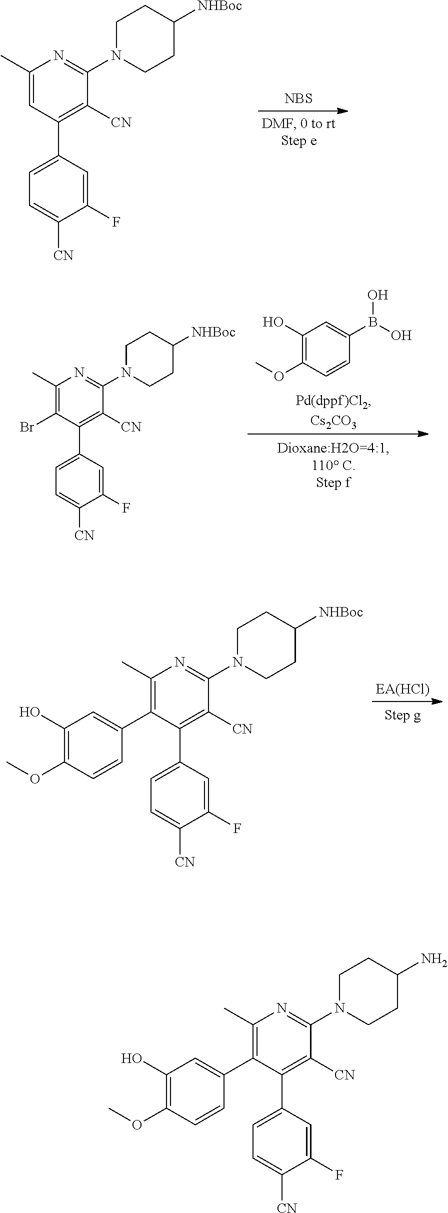 [SMILES: Cc1cc(-c2ccc(C#N)c(F)c2)c(C#N)c(N2CCC(NC(=O)OC(C)(C)C)CC2)n1 ; "NBS" ; Cc1nc(N2CCC(NC(=O)OC(C)(C)C)CC2)c(C#N)c(-c2ccc(C#N)c(F)c2)c1Br ; COc1ccc(B(O)O)cc1O ; COc1ccc(-c2c(C)nc(N3CCC(NC(=O)OC(C)(C)C)CC3)c(C#N)c2-c2ccc(C#N)c(F)c2)cc1O ; COc1ccc(-c2c(C)nc(N3CCC(N)CC3)c(C#N)c2-c2ccc(C#N)c(F)c2)cc1O]

### Step a): Preparation of (2-chloro-4-hydroxy-6-methylpyridine-3-carbonitrile)

(2,4-dichloro-6-methylpyridine-3-carbonitrile) (1.0 g, 5.3 mmol) and cesium acetate (3.0 g, 16.0 mmol) are added to DMF (12 mL) and warmed to 80° C. under nitrogen protection and reacted overnight; when LC-MS monitors that the raw materials disappear, the reaction solution mainly contains the resulting product; when the reaction is completed, the reaction solution is extracted with water and ethyl acetate; an organic phase is washed with saturated saline, dried with anhydrous sodium sulfate, filtered, concentrated, added with silica gel to be mixed, and purified by a normal-phase column to obtain the product (2-chloro-4-hydroxy-6-methylpyridine-3-carbonitrile) with a yield of 95.3%.

ESI-MS m/z=169.2 [M+H]+.

### Step b): Preparation of (tert-butyl N-(1-(3-cyano-4-hydroxy-6-methylpyridin-2-yl)piperidin-4-yl)carbamate)

DIPEA (1.7 g, 13.0 mmol), (2-chloro-4-hydroxy-6-methylpyridine-3-carbonitrile) (1.1 g, 6.5 mmol), and tert-butyl (N-(piperidin-4-yl)carbamate) (1.6 g, 7.8 mmol) are dissolved in NMP (10 mL), warmed to 80° C. and reacted for 2 h. When LC-MS monitors that the raw materials disappear, the reaction solution is extracted with ethyl acetate; the organic phase is washed with saturated saline, dried with anhydrous sodium sulfate, and filtered; the solution is concentrated, added with silica gel to be mixed, and purified by a normal-phase column to obtain the product (tert-butyl N-(1-(3-cyano-4-hydroxy-6-methylpyridin-2-yl)piperidin-4-yl)carbamate) with a yield of 86.2%.

ESI-MS m/z=333.4 [M+H]+.

### Step c): Preparation of 2-(4-((tert-butoxycarbonyl)amino)piperidin-1-yl)-3-cyano-6-methylpyridin-4-yl trifluoromethane sulfonate

(Tert-butyl N-(1-(3-cyano-4-hydroxy-6-methylpyridin-2-yl)piperidin-4-yl)carbamate (500.0 mg, 1.5 mmol) is dissolved in DCE (20.0 mL), added with pyridine (237.3 mg, 3.0 mmol), cooled in an ice bath, then added with trifluoromethanesulfonic anhydride (507.9 mg, 1.8 mmol), and reacted under the ice bath for 1 h; and when TLC (MeOH:DCM=1:8) monitors that the raw materials disappear and a new point is generated, the reaction solution is poured into a sodium bicarbonate aqueous solution and extracted with dichloromethane; organic phase is sequentially washed with a sodium bicarbonate aqueous solution, clean water, and saturated saline, and then concentrated to dryness to remove a solvent, which is used directly for the next reaction.

ESI-MS m/z=464.3 [M+H]+.

### Step d): Preparation of (tert-butyl N-(1-(3-cyano-4-(4-cyano-3-fluorophenyl)-6-methylpyridin-2-yl)piperidin-4-yl)carbamate)

A crude product (400.0 mg, 8.6 mmol) obtained in Step c, ((4-cyano-3-fluorophenyl)boronic acid) (212.7 mg, 1.3 mmol), cesium carbonate (560.4 mg, 1.7 mmol), and Pd(dppf)Cl₂ (62.9 mg, 0.1 mmol) are added to 15 mL of Dioxane/H₂O (4:1), subjected to nitrogen protection, and then reacted in microwaves at 115° C. for 1.5 h; and when LC-MS monitors that the raw materials disappear, the solution is concentrated, and purified by a silica gel column (EA:PE=1:1) to obtain the product (tert-butyl N-(1-(3-cyano-4-(4-cyano-3-fluorophenyl)-6-methylpyridin-2-yl)piperidin-4-yl)carbamate) with a yield of 85.4%.

ESI-MS m/z=436.4 [M+H]+.

### Step e): Preparation of (tert-butyl N-(1-(5-bromo-3-cyano-4-(4-cyano-3-fluorophenyl)-6-methylpyridin-2-yl)piperidin-4-yl)carbam ate)

(Tert-butyl N-(1-(3-cyano-4-(4-cyano-3-fluorophenyl)-6-methylpyridin-2-yl)piperidin-4-yl)carbamate (700 mg, 1.6 mmol) is dissolved in DMF (7.0 mL), added with NBS (515.7 mg, 2.9 mmol), and reacted at room temperature for 1.5 h; the reaction system is added with an aqueous solution of sodium sulfite and extracted with ethyl acetate; the organic phase is washed with water and saturated saline, then concentrated to remove the solvent, mixed and purified by a normal-phase column (EA:PE=2:3) to obtain the product (tert-butyl N-(1-(5-bromo-3-cyano-4-(4-cyano-3-fluorophenyl)-6-methylpyridin-2-yl)piperidin-4-yl)carbam ate) with a yield of 30.45%.

ESI-MS m/z=514.6 [M+H]+.

### Step f): Preparation of (tert-butyl N-(1-(3-cyano-4-(4-cyano-3-fluorophenyl)-5-(3-hydroxy-4-methoxyphenyl)-6-methylpyridin-2-yl)piperidin-4-yl)carbamate)

(Tert-butyl N-(1-(5-bromo-3-cyano-4-(4-cyano-3-fluorophenyl)-6-methylpyridin-2-yl)piperidin-4-yl)carbam ate (80.0 mg, 0.1 mmol), (2-methoxy-5-(tetramethyl-1,3,2-dioxaldehyde-2-yl)phenol) (60.0 mg, 0.2 mmol), cesium carbonate (104.2 mg, 0.3 mmol), and Pd(dppf)Cl₂ (11.7 mg, 0.1 mmol) are added to 2.0 mL of Dioxane/H₂O=4:1, subjected to nitrogen protection, and then reacted in microwaves at 115° C. for 1.5 h; and when LC-MS monitors that the raw materials disappear, the solution is concentrated, and purified by a silica gel column (EA:PE=1:1) to obtain the product (tert-butyl N-(1-(3-cyano-4-(4-cyano-3-fluorophenyl)-5-(3-hydroxy-4-methoxyphenyl)-6-methylpyridin-2-yl)piperidin-4-yl)carbamate) with a yield of 97.0%.

ESI-MS m/z=558.6 [M+H]+.

### Step g): 2-(4-aminopiperidin-1-yl)-4-(4-cyano-3-fluorophenyl)-5-(3-hydroxy-4-methoxyphenyl)-6-methy lpyridine-3-carbonitrile)

(Tert-butyl N-(1-(3-cyano-4-(4-cyano-3-fluorophenyl)-5-(3-hydroxy-4-methoxyphenyl)-6-methylpyridin-2-yl)piperidin-4-yl) carbamate) (35.0 mg, 0.063 mmol) is added with 4N HCl (ethyl acetate) (5 mL), and reacted at room temperature for 0.5 h; and the solution is concentrated to remove a solvent and purified by Prep-HPLC (separation method 4) to obtain (2-(4-aminopiperidin-1-yl)-4-(4-cyano-3-fluorophenyl)-5-(3-hydroxy-4-methoxyphenyl)-6-meth ylpyridine-3-carbonitrile) with a yield of 62.4%.

¹H NMR (400 MHz, DMSO-d₆) δ 8.98 (s, 1H), 7.88 (dd, J=8.0, 6.9 Hz, 1H), 7.53 (dd, J=10.2, 1.4 Hz, 1H), 7.24 (dd, J=8.0, 1.5 Hz, 1H), 6.77 (d, J=8.2 Hz, 1H), 6.59-6.31 (m, 2H), 4.16 (dd, J=12.8, 4.4 Hz, 2H), 3.70 (s, 3H), 3.12 (ddd, J=13.8, 11.2, 2.6 Hz, 2H), 2.84 (tt, J=9.8, 4.1 Hz, 1H), 2.22 (s, 3H), 1.93-1.76 (m, 2H), 1.43-1.24 (m, 2H).

ESI-MS m/z=458.2[M+H]+.

### Example 277

Compound is prepared according to the synthesis method of Example 276 (separation method 4), and its structure and characterization data are as follows:

### 2-(4-aminopiperidin-1-yl)-4-(4-cyano-3-fluorophenyl)-5-(5-fluoro-3-methylbenzo[d]isoxazol-6-yl)-6-methylnicotinonitrile

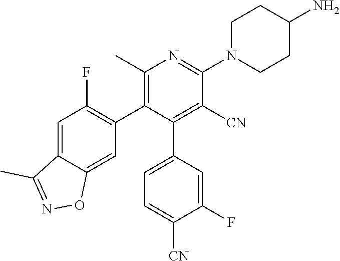 [SMILES: Cc1nc(N2CCC(N)CC2)c(C#N)c(-c2ccc(C#N)c(F)c2)c1-c1cc2onc(C)c2cc1F]

¹H NMR (400 MHz, DMSO-d₆) δ 7.87 (s, 1H), 7.66 (m, 3H), 7.27 (d, J=26.2 Hz, 1H), 4.29 (d, J=13.2 Hz, 2H), 3.20 (t, J=11.8 Hz, 2H), 2.89 (tt, J=9.6, 4.1 Hz, 1H), 2.50 (s, 3H), 2.21 (s, 3H), 1.86 (dd, J=13.6, 3.5 Hz, 2H), 1.36 (dd, J=14.6, 8.8 Hz, 2H).

ESI-MS m/z=485.2[M+H]+.

### Example 278

### Preparation of 4-(4-(4-aminopiperidin-1-yl)-7-(3-hydroxy-4-methoxyphenyl)oxazolo[4,5-c]pyridin-6-yl)-2-fluo robenzonitrile

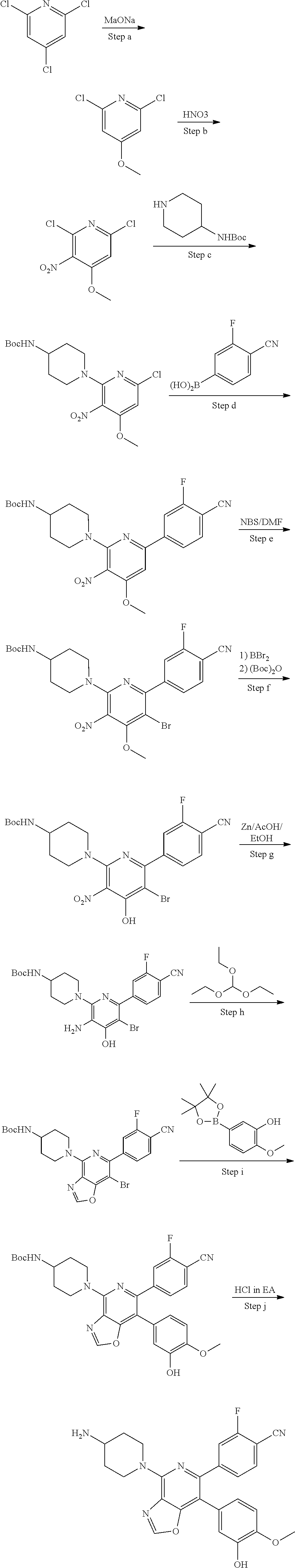

### Step a): Preparation of 2,6-dichloro-4-methoxypyridine

2,4,6-trichloropyridine (10 g, 54.81 mmol) is dissolved in anhydrous methanol (50 mL), subjected to nitrogen protection, cooled to 0° C. in an ice water bath, added with sodium methoxide (2.96 g, 54.81 mmol), and continues to react for 16 h; and LC-MS detection shows that the reaction is completed. The reaction solution is quenched with water (100 mL), and a solid is precipitated, filtered and washed, and concentrated to obtain 2,6-dichloro-4-methoxypyridine with a yield of 93.6.

ESI-MS m/z: 178.0[M+H]⁺.

### Step b): Preparation of 2,6-dichloro-4-methoxy-3-nitropyridine

2,6-dichloro-4-methoxypyridine (5.4 g, 30.33 mmol) is weighed, added with concentrated sulfuric acid (59.6 g, 607.5 mmol, 32.4 mL), cooled to 0° C. in an ice water bath, subjected to nitrogen protection, slowly added with fuming nitric acid (152.3 g, 2.42 mol, 108 mL), warmed to 100° C. under nitrogen protection, and undergoes a reflux reaction for 3 h. The reaction solution is added to ice water for quenching, and a solid is precipitated, suction-filtered and washed, and concentrated and dried to obtain a crude product; and the crude product is purified by silica gel chromatography (eluent: ethyl acetate/petroleum ether=1:5) to obtain 2,6-dichloro-4-methoxy-3-nitropyridine with a yield of 48.2% o.

¹H NMR (400 MHz, DMSO-d₆) δ ppm 7.75 (s, 1H), 4.07 (s, 3H).

ESI-MS m/z: 224.0[M+H]+.

### Step c): Preparation of tert-butyl(1-(6-chloro-4-methoxy-3-nitropyridin-2-yl)piperidin-4-yl)carbamate

2,6-dichloro-4-methoxy-3-nitropyridine (6.22 g, 27.89 mmol), tert-butylpiperidine-4-carbamate (6.14 g, 30.68 mmol), and triethylamine (16.9 g, 167.3 mmol) are weighed and dissolved in dichloromethane (150 mL), subjected to nitrogen protection, and reacted in an ice water bath for 16 h while stirring; when LC-MS detection shows that the reaction is completed, the reaction solution is concentrated to dryness; and the residue is purified by silica gel chromatography (eluent: petroleum ether/ethyl acetate=1: 5) to obtain tert-butyl(1-(6-chloro-4-methoxy-3-nitropyridin-2-yl)piperidin-4-yl)carbamate with a yield of 88.1%.

¹H NMR (400 MHz, DMSO-d₆) δ ppm 6.84 (s, 1H), 3.92 (s, 3H), 3.62 (dd, J=13.4, 4.0 Hz, 2H), 3.48 (s, 1H), 2.99 (ddd, J=13.9, 11.5, 2.7 Hz, 2H), 1.80-1.72 (m, 2H), 1.38 (s, 9H), 1.37-1.31 (m, 2H).

ESI-MS m/z: 387.1[M+H]⁺.

### Step d): Synthesis of tert-butyl(1-(6-(4-cyano-3-fluorophenyl)-4-methoxy-3-nitropyridin-2-yl)piperidin-4-yl)carbamat e

Tert-butyl(1-(6-chloro-4-methoxy-3-nitropyridin-2-yl)piperidin-4-yl) carbamate (7.0 g, 18.10 mmol), (4-cyano-3-fluorophenyl)boronic acid (3.43 g, 20.82 mmol), cesium carbonate (17.69 g, 54.30 mmol), [1,1′-bis(diphenylphosphino)ferrocene]palladium dichloride (660 mg, 0.91 mmol) are weighed and equally divided into 7 parts, which are respectively dissolved in 1,4-Dioxane (10 mL), added with water (2 mL), subjected to nitrogen protection, warmed to 100° C. in microwaves, and reacted for 45 min; when LC-MS detection shows that the reaction is completed, the reaction solution is extracted with ethyl acetate (30 mL×3); organic phase is combined, washed with saturated saline (15 mL×2), dried with anhydrous sodium sulfate, filtered, and concentrated under a reduced pressure; and the residue is purified by silica gel chromatography (eluent: petroleum ether/ethyl acetate=1: 3) to obtain tert-butyl(1-(6-(4-cyano-3-fluorophenyl)-4-methoxy-3-nitropyridin-2-yl)piperidin-4-yl)carbamat e with a yield of 71.2%.

¹H NMR (400 MHz, Chloroform-d) δ 7.87-7.77 (m, 2H), 7.71 (dd, J=7.8, 6.4 Hz, 1H), 6.82 (s, 1H), 4.00 (s, 3H), 3.92-3.83 (m, 2H), 3.70 (s, 1H), 3.10 (ddd, J=13.8, 11.4, 2.6 Hz, 2H), 2.09-1.95 (m, 2H), 1.54-1.47 (m, 2H), 1.45 (s, 9H).

ESI-MS m/z: 472.2 [M+H]⁺.

### Step e): Preparation of tert-butyl(1-(5-bromo-6-(4-cyano-3-fluorophenyl)-4-methoxy-3-nitropyridin-2-yl)piperidin-4-yl) carbamate

### Tert-butyl

(1-(6-(4-cyano-3-fluorophenyl)-4-methoxy-3-nitropyridin-2-yl)piperidin-4-yl)carbamate (5.0 g, 10.60 mmol) is weighed, dissolved in dry DMF (30 mL), added with N-bromosuccinimide (2.83 g, 15.9 mmol), and reacted for 1 h while stirring. When LC-MS monitors that the reaction is completed, the reaction solution is diluted with water (50 mL) and extracted with ethyl acetate (50 mL×3); organic layers are combined and then washed with saturated saline (25 mL×2), dried with anhydrous sodium sulfate, filtered, and concentrated under a reduced pressure to dryness; and the residue is purified by silica gel chromatography (eluent: ethyl acetate/petroleum ether=1:2) to obtain tert-butyl(1-(5-bromo-6-(4-cyano-3-fluorophenyl)-4-methoxy-3-nitropyridin-2-yl)piperidin-4-yl) carbamate with a yield of 97.8%.

¹H NMR (400 MHz, DMSO-d₆) δ ppm 8.08 (dd, J=8.0, 6.8 Hz, 1H), 7.82 (dd, J=10.2, 1.4 Hz, 1H), 7.68 (dd, J=8.0, 1.6 Hz, 1H), 3.99 (s, 3H), 3.69 (d, J=13.4 Hz, 2H), 3.02 (ddd, J=13.8, 11.6, 2.6 Hz, 2H), 1.83-1.70 (m, 2H), 1.42 (s, 2H), 1.37 (s, 9H).

ESI-MS m/z: 550.1 [M+H]⁺.

### Step f): Preparation of tert-butyl(1-(5-bromo-6-(4-cyano-3-fluorophenyl)-4-methoxy-3-nitropyridin-2-yl)piperidin-4-yl) carbamate

Tert-butyl(1-(5-bromo-6-(4-cyano-3-fluorophenyl)-4-methoxy-3-nitropyridin-2-yl)piperidin-4-yl) carbamate (2.0 g, 3.63 mmol) is weighed, dissolved in dry dichloromethane (30 mL), cooled to −78° C. under nitrogen protection, added with boron tribromide (22.7 g, 90.7 mmol), and reacted for 24 h while stirring. In an ice bath, the reaction solution is added dropwise with a saturated sodium bicarbonate aqueous solution (5 mL) for quenching; the system is concentrated under a reduced pressure; the residue is dissolved with tetrahydrofuran (5 mL) and water (5 mL), added with sodium carbonate to adjust the pH to 8, added with di-tert-butyl dicarbonate (3.96 g, 18.15 mmol), and reacted for 1 h while stirring under nitrogen protection; when LC-MS detection shows that the reaction is completed, the reaction solution is diluted with water (10 mL) and extracted with ethyl acetate (30 mL×3); organic phase is combined and then washed with saturated saline (20 mL×2), dried with anhydrous sodium sulfate, filtered, and concentrated under a reduced pressure to dryness; and the residue is purified by silica gel chromatography (eluent: ethyl acetate/petroleum ether=2: 1) to obtain tert-butyl(1-(5-bromo-6-(4-cyano-3-fluorophenyl)-4-methoxy-3-nitropyridin-2-yl)piperidin-4-yl) carbamate with a yield of 58.4%.

ESI-MS m/z: 536.1[M+H]⁺.

### Step g): Preparation of tert-butyl(1-(3-amino-5-bromo-6-(4-cyano-3-fluorophenyl)-4-hydroxypyridin-2-yl)piperidin-4-yl) carbamate

Tert-butyl(1-(5-bromo-6-(4-cyano-3-fluorophenyl)-4-methoxy-3-nitropyridin-2-yl)piperidin-4-yl) carbamate (970 mg, 1.81 mmol) and zinc powder (355.2 mg, 5.43 mmol) are weighed, added with absolute ethanol (5 mL) and acetic acid (1 mL), mixed and dissolved; the system is warmed to 40° C. under nitrogen protection, and reacted for 2 h; and LC-MS detection shows that the reaction is completed. The reaction solution is concentrated to dryness under a reduced pressure, added with saturated sodium bicarbonate (5 mL), then washed with saturated saline (20 mL), dried with anhydrous sodium sulfate, filtered, and concentrated under a reduced pressure to dryness; and the residue is purified by silica gel chromatography (eluent: dichloromethane/ethyl acetate=1:1) to obtain tert-butyl(1-(3-amino-5-bromo-6-(4-cyano-3-fluorophenyl)-4-hydroxypyridin-2-yl)piperidin-4-yl) carbamate with a yield of 68.5%.

ESI-MS m/z: 506.1[M+H]⁺.

### Step h): Preparation of tert-butyl(1-(7-bromo-6-(4-cyano-3-fluorophenyl)oxazole[4,5-c]pyridin-4-yl)piperidin-4-yl)carb amate

Tert-butyl(1-(3-amino-5-bromo-6-(4-cyano-3-fluorophenyl)-4-hydroxypyridin-2-yl)carbamate (200 mg, 0.39 mmol) is weighed and dissolved in triethyl orthoformate (6.23 g, 38.4 mmol, 7 mL); the system is warmed to 100° C. under nitrogen protection, and reacted while stirring; when LC-MS detection shows that the reaction is completed, the reaction solution is concentrated under a reduced pressure to dryness; and the residue is purified by silica gel chromatography (eluent: petroleum ether/ethyl acetate=3: 1) to obtain tert-butyl(1-(7-bromo-6-(4-cyano-3-fluorophenyl)oxazolo[4,5-c]pyridin-4-yl)piperidin-4-yl)carb amate with a yield of 84.9%.

ESI-MS m/z: 516.1[M+H]⁺.

### Step i): Preparation of tert-butyl(1-(6-(4-cyano-3-fluorophenyl)-7-(3-hydroxy-4-methoxyphenyl)oxazolo[4,5-c]pyridin-4-yl)piperidin-4-yl)carbamate

Tert-butyl(1-(7-bromo-6-(4-cyano-3-fluorophenyl)oxazolo[4,5-c]pyridin-4-yl)piperidin-4-yl)carb amate (40 mg, 0.08 mmol), 2-methoxy-5-(4,4,5,5-tetramethyl-1,3,2-dioxaborolane-2-yl)phenol, cesium carbonate (75.3 mg, 0.23 mmol), and [1,1′-bis(diphenylphosphino)ferrocene]palladium dichloride (5.6 mg, 0.01 mmol) are weighed, dissolved in 1,4-dioxane (1.5 mL), added with water (0.3 mL), subjected to nitrogen protection, warmed to 100° C. in microwaves, and reacted for 35 min; when LC-MS detection shows that the reaction is completed, the reaction solution is extracted with ethyl acetate (20 mL×3); organic phase is combined, washed with saturated saline (15 mL×2), dried with anhydrous sodium sulfate, filtered, and concentrated under a reduced pressure; and the residue is purified by silica gel chromatography (eluent: ethyl acetate/petroleum ether=1:2) to obtain ttert-butyl(1-(6-(4-cyano-3-fluorophenyl)-7-(3-hydroxy-4-methoxyphenyl)oxazolo[4,5-c]pyridin-4-yl)piperidin-4-yl)carbamate with a yield of 92.2%.

¹H NMR (400 MHz, DMSO-d₆) δ ppm 9.07 (s, 1H), 8.73 (s, 1H), 7.81 (dd, J=8.2, 7.0 Hz, 1H), 7.46 (dd, J=10.8, 1.6 Hz, 1H), 7.32 (dd, J=82, 1.4 Hz, 1H), 6.88 (dd, J=23.8, 8.2 Hz, 2H), 6.68 (d, J=2.2 Hz, 1H), 6.62 (dd, J=8.2, 2.2 Hz, 1H), 5.01 (d, J=13.4 Hz, 2H), 3.78 (s, 3H), 3.59 (s, 1H), 3.19 (t, J=12.4 Hz, 2H), 1.85 (d, J=11.8 Hz, 2H), 1.46 (d, J=14.8 Hz, 2H), 1.39 (s, 9H).

ESI-MS m/z: 560.2[M+H]⁺.

### Step j): Preparation of 4-(4-(4-aminopiperidin-1-yl)-7-(3-hydroxy-4-methoxyphenyl)oxazolo[4,5-c]pyridin-6-yl)-2-fluo robenzonitrile

Tert-butyl(1-(6-(4-cyano-3-fluorophenyl)-7-(3-hydroxy-4-methoxyphenyl)oxazole[4,5-c]pyridin-4-yl)piperidin-4-yl)carbamate (45 mg, 0.08 mmol) is dissolved in a 4M hydrochloric acid (ethyl acetate solution, 2 mL), and reacted for 60 min at room temperature under nitrogen protection to precipitate solid. When LC-MS detection shows that the reaction is completed, the reaction solution is concentrated to dryness, and is purified by pre-HPLC (separation method 4) to obtain 4-(4-(4-aminopiperidin-1-yl)-7-(3-hydroxy-4-methoxyphenyl)oxazolo[4,5-c]pyridin-6-yl)-2-fluo robenzonitrile with a yield of 36.6%.

¹H NMR (400 MHz, DMSO-d₆) δ ppm 8.71 (s, 1H), 7.81 (dd, J=8.2, 6.8 Hz, 1H), 7.45 (dd, J=10.8, 1.6 Hz, 1H), 7.32 (dd, J=8.2, 1.6 Hz, 1H), 6.91 (d, J=8.4 Hz, 1H), 6.68 (d, J=2.2 Hz, 1H), 6.62 (dd, J=8.2, 2.2 Hz, 1H), 4.96 (dd, J=13.4, 3.6 Hz, 2H), 3.78 (s, 3H), 3.20 (ddd, J=13.8, 11.6, 2.6 Hz, 2H), 2.89 (td, J=9.8, 4.8 Hz, 1H), 1.84 (dd, J=13.2, 3.8 Hz, 2H), 1.29 (qd, J=12.6, 11.8, 7.0 Hz, 2H).

ESI-MS m/z: 460.2 [M+H]⁺.

### Example 279

Compound is prepared according to the synthesis method of Example 278 (separation method 4), and its structure and characterization data are as follows:

### 4-(4-(4-aminopiperidin-1-yl)-7-(5-fluoro-3-methylbenzo[d]isoxazol-6-yl)oxazolo[4,5-c]pyridin-6-yl)-2-fluorobenzonitrile

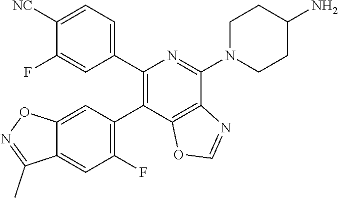 [SMILES: Cc1noc2cc(-c3c(-c4ccc(C#N)c(F)c4)nc(N4CCC(N)CC4)c4ncoc34)c(F)cc12]

¹H NMR (400 MHz, Methanol-d₄) δ ppm 8.25 (s, 1H), 7.53 (d, J=5.4 Hz, 1H), 7.45 (dd, J=8.4, 5.6 Hz, 2H), 7.33 (dd, J=10.4, 1.6 Hz, 1H), 7.18 (dd, J=8.1, 1.5 Hz, 1H), 5.22 (ddd, J=12.6, 7.6, 3.6 Hz, 2H), 3.10 (ddt, J=13.8, 7.8, 2.6 Hz, 2H), 2.94 (tt, J=10.8, 4.2 Hz, 1H), 2.47 (s, 3H), 1.98-1.82 (m, 2H), 1.37 (pd, J=12.2, 4.2 Hz, 2H).

ESI-MS m/z: 487.2 [M+H]⁺.

### Example 280

### Preparation of 4-(5-(4-aminopiperidin-1-yl)-8-(3-hydroxy-4-methoxyphenyl)-3-oxo-3,4-dihydro-2H-pyridine[4,3-b][1,4]oxazin-7-yl)-2-fluorobenzonitrile hydrochloride

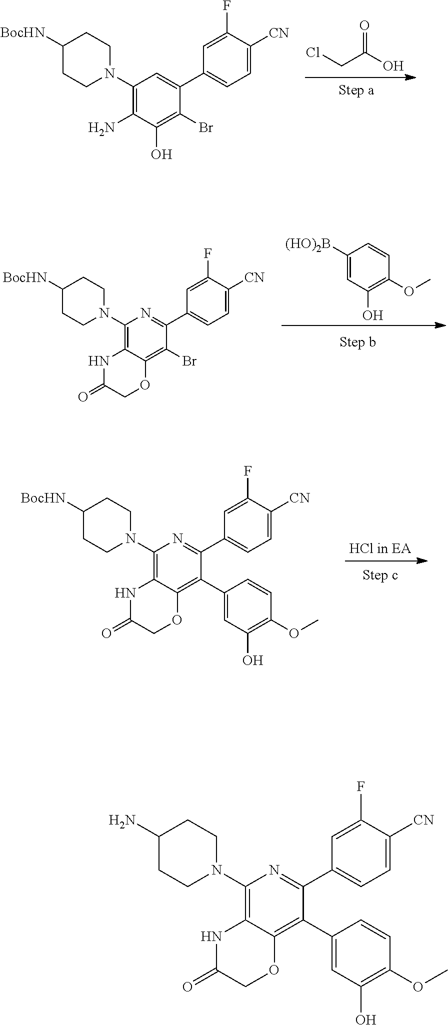 [SMILES: CC(C)(C)OC(=O)NC1CCN(c2cc(-c3ccc(C#N)c(F)c3)c(Br)c(O)c2N)CC1 ; O=C(O)CCl ; CC(C)(C)OC(=O)NC1CCN(c2nc(-c3ccc(C#N)c(F)c3)c(Br)c3c2NC(=O)CO3)CC1 ; COc1ccc(B(O)O)cc1O ; COc1ccc(-c2c(-c3ccc(C#N)c(F)c3)nc(N3CCC(NC(=O)OC(C)(C)C)CC3)c3c2OCC(=O)N3)cc1O ; COc1ccc(-c2c(-c3ccc(C#N)c(F)c3)nc(N3CCC(N)CC3)c3c2OCC(=O)N3)cc1O]

Step a): Preparation of tert-butyl(1-(8-bromo-7-(4-cyano-3-fluorophenyl)-3-oxo-3,4-dihydro-2H-pyrido[4,3-b][1,4]oxaz in-5-yl)piperidin-4-ylcarbamate hydrochloride

### Tert-butyl

(1-(3-amino-5-bromo-6-(4-cyano-3-fluorophenyl)-4-hydroxypyridin-2-yl)piperidin-4-ylcarbamat e (200 mg, 0.39 mmol) and potassium carbonate (161.7 mg, 1.17 mmol) are weighed, dissolved in tetrahydrofuran (10 mL), added with 2-chloroacetyl chloride (66.07 mg, 0.58 mmol) in an ice water bath, reacted for 1 h while stirring, and quenched with saturated sodium bicarbonate solution (1 mL). The reaction solution is extracted with ethyl acetate (20 ml×3); organic phase is combined, washed with saturated saline (15 mL×2), dried with anhydrous sodium sulfate, filtered, concentrated to dryness under a reduced pressure, added with sodium iodide (58.46 mg, 0.39 mmol) and potassium carbonate (107.8 mg, 0.78 mmol), dissolved in dry acetonitrile (10 mL), warmed to 60° C., and reacted for 3 h. When LC-MS detection shows that the reaction is completed, the reaction solution is concentrated to dryness; and the residue is purified by silica gel chromatography (eluent: ethyl acetate/petroleum ether=1:1); and the eluent is concentrated to obtain tert-butyl(1-(8-bromo-7-(4-cyano-3-fluorophenyl)-3-oxo-3,4-dihydro-2H-pyrido[4,3-b][1,4]oxaz in-5-yl)piperidin-4-yl-carbamate hydrochloride with a yield of 71.3%.

¹H NMR (400 MHz, DMSO-d₆) δ ppm 10.44 (s, 1H), 8.02 (dd, J=8.2, 6.8 Hz, 1H), 7.77 (dd, J=10.4, 1.6 Hz, 1H), 7.70 (dd, J=8.2, 1.6 Hz, 1H), 6.90 (d, J=7.4 Hz, 1H), 4.79 (s, 2H), 3.52 (d, J=13.0 Hz, 2H), 3.40 (s, 1H), 2.84 (t, J=11.4 Hz, 2H), 1.74 (d, J=12.0 Hz, 2H), 1.64 (td, J=11.2, 7.6 Hz, 2H), 1.39 (s, 9H).

ESI-MS m/z: 546.1[M+H]⁺.

### Step b): Preparation of tert-butyl(1-(7-(4-cyano-3-fluorophenyl)-8-(3-hydroxy-4-methoxyphenyl)-3-oxo-3,4-dihydro-2H -pyrido[4,3-b][1,4]oxazin-5-yl)piperidin-4-yl)carbamate

Tert-butyl(1-(8-bromo-7-(4-cyano-3-fluorophenyl)-3-oxo-3,4-dihydro-2H-pyrido[4,3-b][1,4]oxa zin-5-yl)piperidin-4-yl-carbamate (60.0 mg, 0.11 mmol), 3-hydroxy-4-methoxyphenylboronic acid (27.7 mg, 0.17 mmol), cesium carbonate (107.5 mg, 0.33 mmol), and [1,1′-bis(diphenylphosphino)ferrocene]palladium dichloride (8.1 mg, 0.01 mmol) are weighed, dissolved in 1,4-dioxane (3 mL), added with water (0.6 mL), subjected to nitrogen protection, warmed to 120° C. in microwaves, and reacted for 30 min; when LC-MS detection shows that the reaction is completed, the reaction solution is extracted with ethyl acetate (20 mL×3); organic phase is combined, washed with saturated saline (15 mL×2), dried with anhydrous sodium sulfate, filtered, and concentrated under a reduced pressure; and the residue is purified by silica gel chromatography (eluent: ethyl acetate/petroleum ether=1:1) to obtain tert-butyl(1-(7-(4-cyano-3-fluorophenyl)-8-(3-hydroxy-4-methoxyphenyl)-3-oxo-3,4-dihydro-2H-pyrido[4,3-b][1,4]oxazin-5-yl)piperidin-4-yl)carbamate with a yield of 92.5%.

ESI-MS m/z: 590.2[M+H]⁺.

### Step c): Preparation of 4-(5-(4-aminopiperidin-1-yl)-8-(3-hydroxy-4-methoxyphenyl)-3-oxo-3,4-dihydro-2H-pyridine[4,3-b][1,4]oxazin-7-yl)-2-fluorobenzonitrile hydrochloride

Tert-butyl(1-(7-(4-cyano-3-fluorophenyl)-8-(3-hydroxy-4-methoxyphenyl)-3-oxo-3,4-dihydro-2H-pyrido[4,3-b][1,4]oxazin-5-yl)piperidin-4-yl)carbamate (60 mg, 0.09 mmol) is added to a 4M hydrochloric acid (ethyl acetate solution, 1 mL), and reacted for 30 min under nitrogen protection to precipitate solid; and LC-MS detection shows that the reaction is completed. The reaction solution is concentrated to dryness and freeze-dried to obtain 4-(5-(4-aminopiperidin-1-yl)-8-(3-hydroxy-4-methoxyphenyl)-3-oxo-3,4-dihydro-2H-pyrido[4,3-b][1,4]oxazin-7-yl)-2-fluorobenzonitrile hydrochloride with a yield of 98.7%.

¹H NMR (400 MHz, DMSO-d₆) δ ppm 10.36 (s, 1H), 9.01 (s, 1H), 8.17-7.95 (br, 3H), 7.75 (dd, J=8.2, 7.0 Hz, 1H), 7.36 (dd, J=11.0, 1.4 Hz, 1H), 7.18 (dd, J=8.2, 1.6 Hz, 1H), 6.86 (d, J=8.2 Hz, 1H), 6.59 (d, J=2.2 Hz, 1H), 6.48 (dd, J=8.2, 2.2 Hz, 1H), 4.60 (s, 2H), 3.76 (s, 3H), 3.63 (d, J=12.8 Hz, 2H), 3.24-3.14 (m, 1H), 3.00-2.82 (m, 2H), 2.00-1.89 (m, 2H), 1.84 (dt, J=11.8, 5.9 Hz, 2H).

ESI-MS m/z: 490.2 [M+H]⁺.

### Example 281

Compound is prepared according to the synthesis method of Example 280 (separation method 4), and its structure and characterization data are as follows:

### 4-(5-(4-aminopiperidin-1-yl)-8-(5-fluoro-3-methylbenzo[d]isoxazol-6-yl)-3,4-dihydro-2H-pyri[4,3-b][1,4]oxazol-7-yl)-2-fluorobenzonitrile

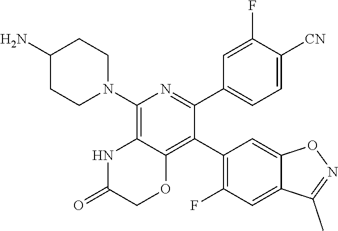 [SMILES: Cc1noc2cc(-c3c(-c4ccc(C#N)c(F)c4)nc(N4CCC(N)CC4)c4c3OCC(=O)N4)c(F)cc12]

¹H NMR (400 MHz, Methanol-d4) δ ppm 7.51 (td, J=8.2, 3.2 Hz, 3H), 7.39-7.32 (m, 1H), 7.16 (dd, J=8.2, 1.6 Hz, 1H), 4.68-4.60 (m, 2H), 3.79-3.65 (m, 2H), 3.09-2.94 (m, 3H), 2.56 (s, 3H), 1.96 (d, J=12.0 Hz, 2H), 1.85-1.67 (m, 2H).

ESI-MS m/z: 517.2 [M+H]⁺.

### Example 282

### Preparation of 2-(4-aminopiperidin-1-yl)-6-(4-cyano-3-fluorophenyl)-5-(3-hydroxy-4-methoxyphenyl)nicotinon itrile hydrochloride

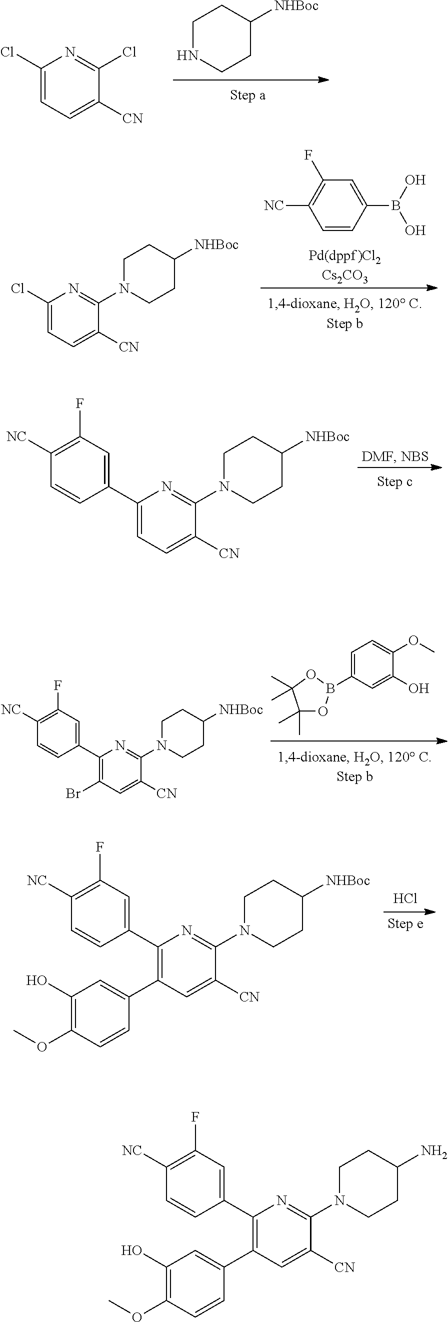

Step a): Preparation of tert-butyl (1-(6-chloro-3-cyanopyridin-2-yl)piperidin-4-yl)carbamate 2,6-dichloronicotinonitrile (2 g, 11.53 mmol), tert-butyl piperidin-4-yl-carbamate (2.54 g, 12.68 mmol) and triethylamine (3.5 g, 34.59 mmol) are added to a reaction flask containing NMP (20 mL), and the mixture is reacted for 16 h at room temperature while stirring. The reaction solution is quenched with water (20 mL), and extracted with ethyl acetate (30 mL×2); organic phase is combined, washed with saturated saline (30 mL×3), dried with anhydrous sodium sulfate, and filtered; and the residue is purified by silica gel chromatography (eluent: petroleum ether/ethyl acetate=2/1) to obtain tert-butyl (1-(6-chloro-3-cyanopyridin-2-yl)piperidin-4-yl)carbamate with a yield of 57.1%.

¹H NMR (400 MHz, DMSO-d6) δ ppm 7.90 (d, J=9.0 Hz, 1H), 6.93 (d, J=9.0 Hz, 1H), 4.32-4.19 (m, 2H), 3.58 (d, J=10.6 Hz, 1H), 3.11 (ddd, J=14.0, 11.6, 2.8 Hz, 2H), 1.85-1.76 (m, 2H), 1.39 (s, 9H), 1.35-1.25 (m, 2H).

ESI-MS m/z=337.2 [M+H]+.

### Step b): Preparation of tert-butyl (1-(3-cyano-4-(4-cyano-3-fluorophenyl)pyridin-2-yl)piperidin-4-yl)carbamate

Tert-butyl (1-(6-chloro-3-cyanopyridin-2-yl)piperidin-4-yl)carbamate (400 mg, 1.19 mmol), 4-cyano-3-fluorophenylboronic acid (0.22 g, 1.31 mmol), Pd(dppf)Cl₂ (0.087 g, 0.12 mmol) and cesium carbonate (0.78 g, 2.38 mmol) are added to a microwave tube containing 1,4-dioxane (8 mL) and water (1 mL), and the mixture is stirred at 120° C. in microwaves for 1 h. The reaction solution is then added with silica gel and concentrated; and the residue is purified by silica gel chromatography (eluent: petroleum ether/ethyl acetate=1/1) to obtain tert-butyl (1-(3-cyano-4-(4-cyano-3-fluorophenyl)pyridin-2-yl)piperidin-4-yl)carbamate with a yield of 95.7%.

¹H NMR (400 MHz, DMSO-d6) δ ppm 8.11 (s, 1H), 8.01-7.90 (m, 2H), 7.86 (dd, J=8.2, 1.8 Hz, 1H), 7.05 (d, J=9.2 Hz, 1H), 6.88 (d, J=7.8 Hz, 1H), 4.40 (d, J=13.4 Hz, 2H), 3.32 (d, J=1.6 Hz, 1H), 3.18-3.07 (m, 2H), 1.87-1.77 (m, 2H), 1.39 (s, 9H), 1.36-1.27 (m, 2H).

ESI-MS m/z=422.2 [M+H]+.

### Step c): Preparation of tert-butyl (1-(5-bromo-3-cyano-6-(4-cyano-3-fluorophenyl)pyridin-2-yl)piperidin-4-yl)carbamate

Tert-butyl (1-(3-cyano-6-(4-cyano-3-fluorophenyl)pyridin-2-yl)piperidin-4-yl)carbamate (460 mg, 1.09 mmol) and NBS (0.23 g, 1.31 mmol) are added to a reaction flask containing DMF (15 mL), and the mixture is stirred for 2 h at room temperature. The reaction solution is quenched with water (20 mL), and extracted with ethyl acetate (30 mL×2); organic phase is combined, washed with saturated saline (30 mL×2), dried with anhydrous sodium sulfate, and filtered; and the residue is purified by silica gel chromatography (eluent: petroleum ether/ethyl acetate=2/1) to obtain tert-butyl (1-(5-bromo-3-cyano-6-(4-cyano-3-fluorophenyl)pyridin-2-yl)piperidin-4-yl)carbamate with a yield of 75.9%.

¹H NMR (400 MHz, DMSO-d6) δ ppm 8.56 (d, J=1.2 Hz, 1H), 8.20-8.09 (m, 1H), 7.98 (dt, J=10.4, 1.4 Hz, 1H), 7.94-7.85 (m, 1H), 6.92 (d, J=8.0 Hz, 1H), 4.12 (d, J=13.4 Hz, 2H), 3.13-3.00 (m, 2H), 1.93-1.79 (m, 2H), 1.58-1.47 (m, 2H), 1.39 (s, 9H).

ESI-MS m/z=500.1 [M+H]+.

### Step d): Preparation of tert-butyl (1-(3-cyano-4-(4-cyano-3-fluorophenyl)-5-(3-hydroxy-4-methoxyphenyl)pyridin-2-yl)piperidin-4-yl)carbamate

### Tert-butyl

(1-(5-bromo-3-cyano-3-cyano-6-(4-cyano-3-fluorophenyl)pyridin-2-yl)piperidin-4-yl)carbamate (80 mg, 0.16 mmol), Pd(Dppf)Cl₂ (12 mg, 0.016 mmol), cesium carbonate (0.10 g, 0.32 mmol) and 2-methoxy-5-(4,4,5,5-tetramethyl-1,3,2-dioxaborolane-2-yl)phenol (44 mg, 0.18 mmol) are added to a microwave tube containing 1,4-dioxane (4 mL) and water (1 mL), and the mixture is stirred at 120° C. in microwaves for 1 h under nitrogen protection. The reaction solution is then added with silica gel and concentrated; and the residue is purified by silica gel chromatography (eluent: petroleum ether/ethyl acetate=1/1) to obtain tert-butyl (1-(3-cyano-4-(4-cyano-3-fluorophenyl)-5-(3-hydroxy-4-methoxyphenyl)pyridin-2-yl)piperidin-4-yl)carbamate with a yield of 97.0%.

ESI-MS m/z=544.3 [M+H]+.

### Step e): Preparation of 2-(4-aminopiperidin-1-yl)-6-(4-cyano-3-fluorophenyl)-5-(3-hydroxy-4-methoxyphenyl)nicotinon itrile hydrochloride

### Tert-butyl

(1-(3-cyano-6-(4-cyano-3-fluorophenyl)-5-(3-hydroxy-4-methoxyphenyl)pyridin-2-yl)piperidin-4-yl)carbamate (80 mg, 0.15 mmol) and 4M hydrochloric acid (ethyl acetate solution, 5.5 mg, 0.15 mmol) are added to a reaction flask, and the mixture is stirred for 1 h at room temperature. The reaction solution is concentrated, and then delivered to Prep-HPLC preparation (separation method 1) to obtain 2-(4-aminopiperidin-1-yl)-6-(4-cyano-3-fluorophenyl)-5-(3-hydroxy-4-methoxyphenyl)nicotinon itrile hydrochloride with a yield of 97.11%.

¹H NMR (400 MHz, DMSO-d6) δ ppm 9.33 (s, 1H), 8.22-8.13 (br, 3H), 8.03 (dd, J=10.4, 1.6 Hz, 1H), 7.97 (dd, J=8.1, 1.6 Hz, 1H), 7.94 (s, 1H), 7.11-6.92 (m, 3H), 3.86 (d, J=14.2 Hz, 2H), 3.82 (s, 3H), 3.22 (tq, J=10.6, 4.8 Hz, 1H), 2.93-2.78 (m, 2H), 1.85 (dd, J=13.0, 4.2 Hz, 2H), 1.62 (tt, J=12.0, 6.2 Hz, 2H).

ESI-MS m/z=444.2 [M+H]+.

### Example 283

### Preparation of 2-(4-aminopiperidin-1-yl)-5-(4-cyano-3-fluorophenyl)-6-(3-hydroxy-4-methoxyphenyl)nicotinon itrile hydrochloride

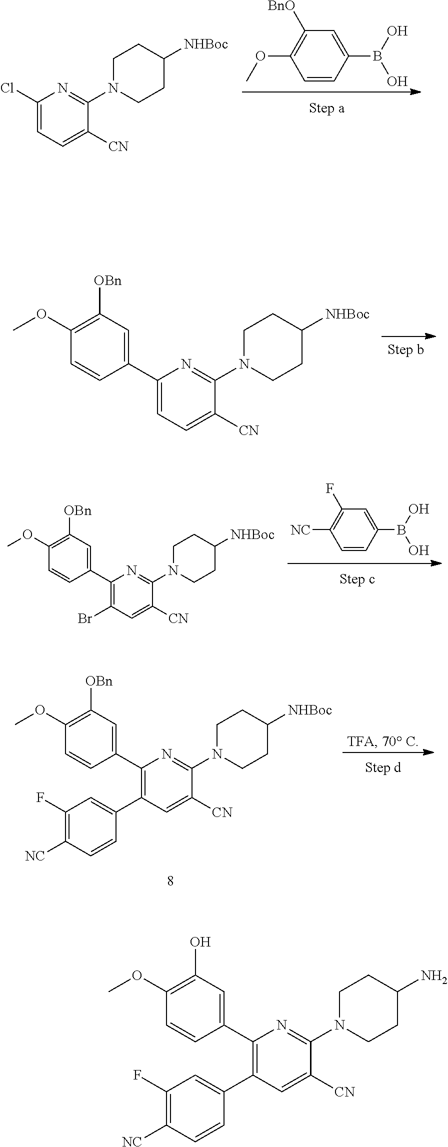

### Step a): Preparation of tert-butyl (1-(6-(3-(benzyloxy)-4-methoxyphenyl)-3-cyanopyridin-2-yl)piperidin-4-yl)carbamate hydrochloride

Tert-butyl (1-(6-chloro-3-cyanopyridin-2-yl)piperidin-4-yl)carbamate (400 mg, 1.19 mmol), (3-(benzyloxy)-4-methoxyphenyl)boronic acid (0.34 g, 1.31 mmol), Pd(dppf)Cl₂ (87 mg, 0.12 mmol) and cesium carbonate (0.78 g, 2.38 mmol) are added to a microwave tube containing 1,4-dioxane (8 mL) and water (2 mL), and the mixture is stirred at 120° C. in microwaves for 1 h. The reaction solution is then added with silica gel and concentrated; and the residue is purified by silica gel chromatography (eluent: petroleum ether/ethyl acetate=1/1) to obtain tert-butyl (1-(6-(3-(benzyloxy)-4-methoxyphenyl)-3-cyanopyridin-2-yl)piperidin-4-yl)carbamate with a yield of 99.4%.

¹H NMR (400 MHz, DMSO-d6) δ ppm 7.86 (d, J=9.0 Hz, 1H), 7.51-7.37 (m, 4H), 7.34 (dd, J=6.8, 1.8 Hz, 2H), 7.12 (d, J=8.6 Hz, 1H), 6.89 (s, 1H), 6.87 (s, 1H), 5.18 (s, 2H), 4.34 (d, J=13.4 Hz, 2H), 3.86 (s, 3H), 3.32 (s, 1H), 3.12-3.02 (m, 2H), 1.86-1.76 (m, 2H), 1.40 (s, 9H), 1.32 (dd, J=12.4, 3.6 Hz, 2H).

ESI-MS m/z=515.3 [M+H]+.

### Step b): Preparation of tert-butyl (1-(6-(3-(benzyloxy)-4-methoxyphenyl)-5-bromo-3-cyanopyridin-2-yl)piperidin-4-yl)carbamate

### Tert-butyl

(1-(6-(3-(benzyloxy)-4-methoxyphenyl)-3-cyanopyridin-2-yl)piperidin-4-yl)carbamate (580 mg, 1.13 mmol) and NBS (0.24 g, 1.36 mmol) are added to a reaction flask containing DMF (15 mL), and the mixture is stirred for 2 h at room temperature. The reaction solution is quenched with water (20 mL), and extracted with ethyl acetate (30 mL×2); organic phase is combined, washed with saturated saline (30 mL×2), dried with anhydrous sodium sulfate, and filtered; and the residue is purified by silica gel chromatography (eluent: petroleum ether/ethyl acetate=2/1) to obtain tert-butyl (1-(6-(3-(benzyloxy)-4-methoxyphenyl)-5-bromo-3-cyanopyridin-2-yl)piperidin-4-yl)carbamate with a yield of 98.5%.

¹H NMR (400 MHz, DMSO-d6) δ ppm 8.40 (s, 1H), 7.55 (dd, J=6.0, 2.3 Hz, 2H), 7.48-7.44 (m, 2H), 7.39 (t, J=7.4 Hz, 2H), 7.36-7.31 (m, 1H), 7.19-7.11 (m, 1H), 6.91 (d, J=8.0 Hz, 1H), 5.16 (s, 2H), 4.09-3.99 (m, 2H), 3.86 (s, 3H), 3.34 (d, J=1.4 Hz, 1H), 3.08-2.96 (m, 2H), 1.85 (d, J=11.6 Hz, 2H), 1.52 (dt, J=12.4, 7.2 Hz, 2H), 1.39 (s, 9H).

ESI-MS m/z=593.2 [M+H]+.

### Step c): Preparation of tert-butyl(1-(6-(3-(benzyloxy)-4-methoxyphenyl)-3-cyano-5-(4-cyano-3-fluorophenyl)pyridin-2-yl)piperidin-4-yl)carbamate

Tert-butyl(1-(6-(3-(benzyloxy)-4-methoxyphenyl)-5-bromo-3-cyanopyridin-2-yl)piperidin-4-yl)c arbamate (200 mg, 0.34 mmol), Pd(dppf)Cl₂ (25 mg, 0.034 mmol), cesium carbonate (0.22 g, 0.68 mmol) and (4-cyano-3-fluorophenyl)boronic acid (62 mg, 0.37 mmol) are added to a microwave tube containing 1,4-Dioxane (4 mL) and water (1 mL), and the mixture is stirred at 120° C. in microwaves for 1 h under nitrogen protection. The reaction solution is then added with silica gel and concentrated; and the residue is purified by silica gel chromatography (eluent: petroleum ether/ethyl acetate=1/1) to obtain tert-butyl(1-(6-(3-(benzyloxy)-4-methoxyphenyl)-3-cyano-5-(4-cyano-3-fluorophenyl)pyridin-2-yl)piperidin-4-yl)carbamate with a yield of 98.8%.

ESI-MS m/z=634.3 [M+H]+.

### Step d): Preparation of 2-(4-aminopiperidin-1-yl)-5-(4-cyano-3-fluorophenyl)-6-(3-hydroxy-4-methoxyphenyl)nicotinon itrile hydrochloride

### Tert-butyl

(1-(6-(3-(benzyloxy)-4-methoxyphenyl)-3-cyano-5-(4-cyano-3-fluorophenyl)pyridin-2-yl)piperi din-4-yl)carbamate (165 mg, 0.26 mmol) and trifluoroacetic acid (30 mg, 0.26 mmol) are added to a reaction flask, and the mixture is stirred at 70° C. for 16 h. The reaction solution is concentrated, and then purified by Prep-HPLC (separation method 1) to obtain 2-(4-aminopiperidin-1-yl)-5-(4-cyano-3-fluorophenyl)-6-(3-hydroxy-4-methoxyphenyl)nicotinon itrile hydrochloride with a yield of 94.0%.

¹H NMR (400 MHz, DMSO-d6) δ ppm 9.38 (s, 1H), 9.07 (s, 1H), 8.11 (s, 1H), 8.09 (d, J=6.7 Hz, 2H), 8.03 (dd, J=8.2, 7.0 Hz, 1H), 7.81 (dd, J=10.8, 1.6 Hz, 1H), 7.64 (dd, J=8.2, 1.6 Hz, 1H), 7.50-7.43 (m, 2H), 7.13-7.06 (m, 1H), 3.75 (d, J=13.4 Hz, 3H), 3.23 (d, J=1.6 Hz, 2H), 2.91 (s, 1H), 2.88 (d, J=2.4 Hz, 2H), 1.85 (s, 2H), 1.58-1.46 (m, 2H).

ESI-MS m/z=444.2 [M+H]+.

### Example 284

Compound is prepared according to the synthesis method of Example 283 (separation method 1), and its structure and characterization data are as follows: 2-(4-aminopiperidin-1-yl)-6-(4-cyano-3-fluorophenyl)-5-(5-fluoro-3-methylbenzo[d]isoxazol-6-yl)nicotinonitrile hydrochloride

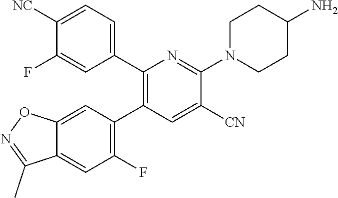 [SMILES: Cc1noc2cc(-c3cc(C#N)c(N4CCC(N)CC4)nc3-c3ccc(C#N)c(F)c3)c(F)cc12]

¹H NMR (400 MHz, Methanol-d₄) δppm 7.98-7.77 (m, 4H), 7.72 (d, J=5.4 Hz, 1H), 7.62 (d, J=8.8 Hz, 1H), 3.92 (dp, J=14.0, 2.2 Hz, 2H), 3.20-3.10 (m, 1H), 2.86 (ddd, J=14.2, 12.2, 2.2 Hz, 2H), 2.50 (s, 3H), 1.89-1.73 (m, 2H), 1.43 (qd, J=12.2, 4.1 Hz, 2H).

ESI-MS m/z=470.2 [M+H]⁺.

### Example 285

### Preparation of 4-(7-(4-aminopiperidin-1-yl)-4-(3-hydroxy-4-methoxyphenyl)-1-methyl-1H-pyrazolo[3,4-c]pyri din-5-yl)-2-fluorobenzonitrile

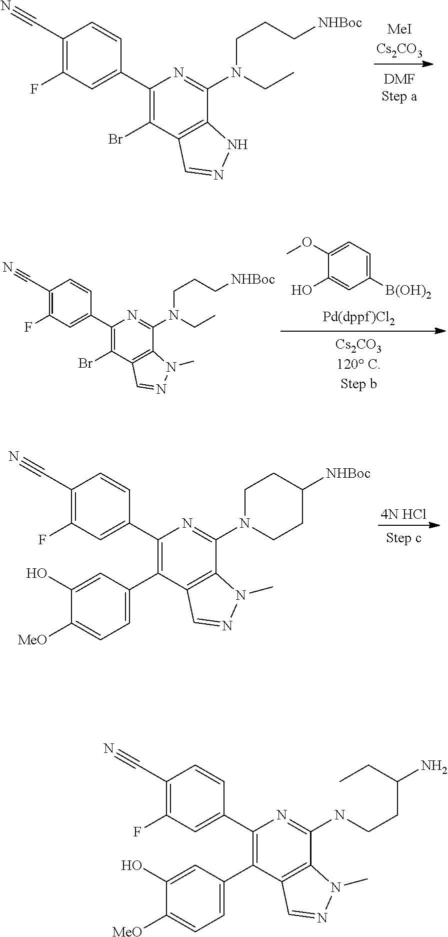

### Step a): Preparation of tert-butyl(1-(4-bromo-5-(4-cyano-3-fluorophenyl)-1-methyl-1H-pyrazolo[3,4-c]pyridin-7-yl)pipe ridin-4-yl)carbamate

Tert-butyl(1-(4-bromo-5-(4-cyano-3-fluorophenyl)-1H-pyrazolo[3,4-c]pyridin-7-yl)piperidin-4-y 1)carbamate (200 mg, 0.39 mmol), methyl iodide (83 mg, 0.58 mmol), cesium carbonate (254 mg, 0.78 mmol), and DMF (10 mL) are added to a microwave reactor and reacted while stirring at 40° C. for 2 h. When the reaction is completed, the reaction solution is added with water (20 mL), and extracted with ethyl acetate (20 mL×2); organic phase is combined, washed with saturated saline (15 mL×2), dried with anhydrous sodium sulfate, filtered and concentrated under a reduced pressure; and the residue is purified by silica gel chromatography (eluent: petroleum ether/ethyl acetate=10/3) to obtain tert-butyl(1-(4-bromo-5-(4-cyano-3-fluorophenyl)-1-methyl-1H-pyrazole[3,4-c]pyridin-7-yl)pipe ridin-4-yl)carbamate with a yield of 63.0%.

ESI-MS(m/z)=529.1[M+H]⁺.

### Step b): Preparation of tert-butyl(1-(5-(4-cyano-3-fluorophenyl)-4-(3-hydroxy-4-methoxyphenyl)-1-methyl-1H-pyrazole [3,4-c]pyridin-7-yl)piperidin-4-yl)carbamate

Tert-butyl(1-(4-bromo-5-(4-cyano-3-fluorophenyl)-1-methyl-1H-pyrazole[3,4-c]pyridin-7-yl)pip eridin-4-yl)carbamate (130 mg, 0.25 mmol), (3-(benzyloxy)-4-methoxyphenyl)boronic acid (61 mg, 0.38 mmol), Cs₂CO₃ (163 mg, 0.50 mmol), Pd(dppf)Cl₂ (22 mg, 0.03 mmol), 1.4-Dioxane (10 mL) and H₂O (2.5 mL) are added to a reaction flask, and reacted while stirring at 120° C. for 1 h. The reaction solution is concentrated under a reduced pressure to dryness; and the residue is purified by silica gel chromatography (eluent: petroleum ether/ethyl acetate=4/3) to obtain tert-butyl(1-(5-(4-cyano-3-fluorophenyl)-4-(3-hydroxy-4-methoxyphenyl)-1-methyl-1H-pyrazol o[3,4-c]pyridin-7-yl)piperidin-4-yl)carbamate with a yield of 56.0%.

ESI-MS(m/z)=573.2[M+H]⁺.

### Step c): Preparation of 4-(7-(4-aminopiperidin-1-yl)-4-(3-hydroxy-4-methoxyphenyl)-1-methyl-H-pyrazolo[3,4-c]pyri din-5-yl)-2-fluorobenzonitrile

Tert-butyl(1-(5-(4-cyano-3-fluorophenyl)-4-(3-hydroxy-4-methoxyphenyl)-1-methyl-1H-pyrazol e[3,4-c]pyridin-7-yl)piperidin-4-yl)carbamate (80 mg, 0.14 mmol) is added to a reaction flask, then added with 4M hydrogen chloride (ethyl acetate solution, 2.5 mL) and stirred at room temperature for 1 h; a large amount of solids precipitate, and then concentrated under a reduced pressure to obtain a crude product; and the crude product is purified by Prep-HPLC (separation method 1) to obtain 4-(7-(4-aminopiperidin-1-yl)-4-(3-hydroxy-4-methoxyphenyl)-1-methyl-1H-pyrazolo[3,4-c]pyri din-5-yl)-2-fluorobenzonitrile with a yield of 42.5%.

¹H NMR (400 MHz, DMSO-d₆+D₂0) δ ppm 8.21 (s, 1H), 7.77 (t, J=7.6 Hz, 1H), 7.49 (dd, J=11.0, 1.6 Hz, 1H), 7.33 (dd, J=8.0, 1.6 Hz, 1H), 6.94 (d, J=8.2 Hz, 1H), 6.63 (d, J=7.4 Hz, 2H), 5.21 (d, J=13.6 Hz, 2H), 4.18 (s, 3H), 3.79 (s, 3H), 3.44 (dq, J=11.4, 6.6, 5.4 Hz, 1H), 3.30 (t, J=12.8 Hz, 2H), 2.20-1.98 (m, 2H), 1.67 (qd, J=12.4, 4.0 Hz, 2H).

ESI-MS(m/z)=473.2 [M+H]⁺.

### Example 286

Compound is prepared according to the synthesis method of Example 285 (separation method 1), and its structure and characterization data are as follows:

### 4-(7-(4-aminopiperidin-1-yl)-4-(5-fluoro-3-methylbenzo[d]isoxazol-6-yl)-1-methyl-1H-pyrazolo [3,4-c]pyridin-5-yl)-2-fluorobenzonitrile

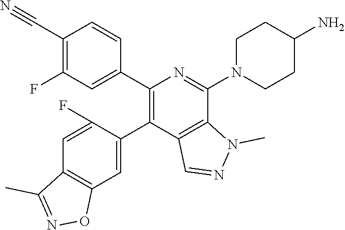 [SMILES: Cc1noc2cc(-c3c(-c4ccc(C#N)c(F)c4)nc(N4CCC(N)CC4)c4c3cnn4C)c(F)cc12]

¹H NMR (400 MHz, Methanol-d₄) δ ppm 7.82 (d, J=11.8 Hz, 1H), 7.52 (d, J=5.4 Hz, 1H), 7.45-7.24 (m, 3H), 7.22-7.11 (m, 1H), 5.33 (t, J=14.4 Hz, 2H), 4.08 (s, 3H), 3.16-3.01 (m, 2H), 3.01-2.88 (m, 1H), 2.47 (s, 3H), 1.88 (dd, J=17.4, 13.4 Hz, 2H), 1.54-1.31 (m, 2H).

ESI-MS(m/z)=500.2 [M+H]⁺.

### Example 287

### Preparation of 4-(7-(4-aminopiperidin-1-yl)-4-(3-hydroxy-4-methoxyphenyl)-1H-pyrazolo[3,4-c]pyridin-5-yl)-2-fluorobenzonitrile hydrochloride

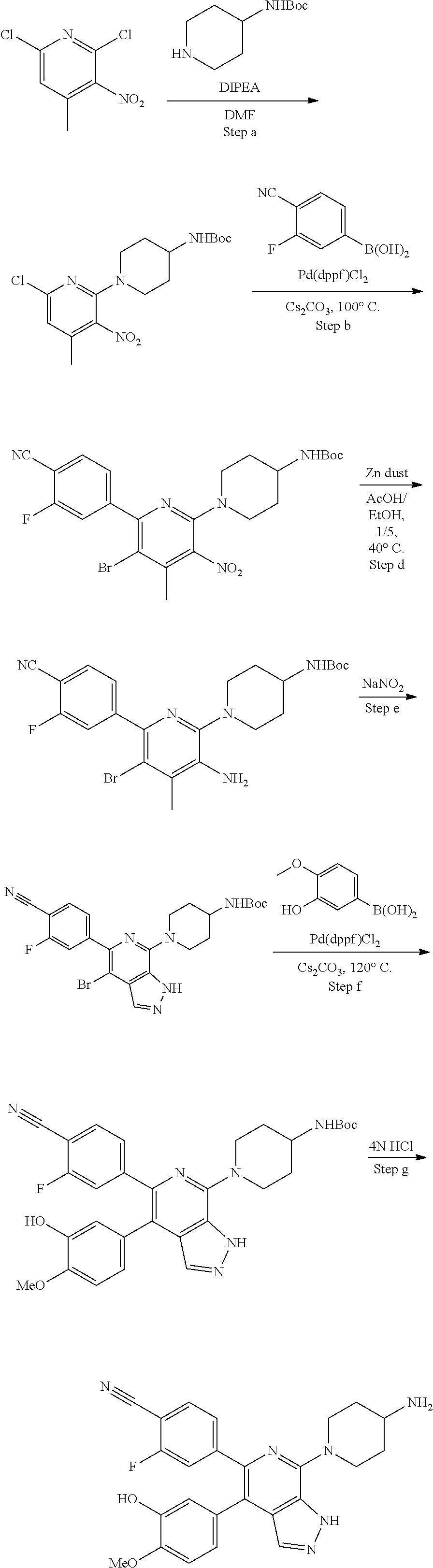

### Step a): Preparation of tert-butyl(1-(6-chloro-4-methyl-3-nitropyridin-2-yl)piperidin-4-yl)carbamate

2,6-dichloro-4-methyl-3-nitropyridine (500 mg, 2.4 mmol), tert-butyl piperidin-4-yl-carbamate (728 mg, 3.6 mmol) and NMP (10 mL) are added to a microwave reactor and reacted while stirring at 80° C. for 5 h. When the reaction is completed, the reaction solution is added with water (20 mL), and extracted with ethyl acetate (20 mL×2); organic phase is combined, washed with saturated saline (15 mL×2), dried with anhydrous sodium sulfate, filtered and concentrated under a reduced pressure; and the residue is purified by silica gel chromatography (eluent: petroleum ether/ethyl acetate=10/3) to obtain tert-butyl(1-(6-chloro-4-methyl-3-nitropyridin-2-yl)piperidin-4-yl)carbamate with a yield of 63.0%.

ESI-MS(m/z)=371.1[M+H]⁺.

### Step b): Preparation of tert-butyl(1-(5-bromo-6-(4-cyano-3-fluorophenyl)-4-methyl-3-nitropyridin-2-yl)piperidin-4-yl)c arbamate

Tert-butyl(1-(6-chloro-4-methyl-3-nitropyridin-2-yl)piperidin-4-yl)carbamate (561 mg, 1.5 mmol), (4-cyano-3-fluorophenyl) boronic acid (371 mg, 2.3 mmol), Cs₂CO₃ (978 mg, 3.0 mmol), Pd(dppf)Cl₂ (110 mg, 0.15 mmol), 1.4-dioxane (10 mL) and H₂O (2.5 mL) are added to a reaction flask, and reacted while stirring at 120° C. for 1 h. The reaction solution is concentrated under a reduced pressure to dryness; and the residue is purified by silica gel chromatography (eluent: petroleum ether/ethyl acetate=8/3) to obtain tert-butyl(1-(5-bromo-6-(4-cyano-3-fluorophenyl)-4-methyl-3-nitropyridin-2-yl)piperidin-4-yl)c arbamate with a yield of 57.0%.

ESI-MS(m/z)=456.2[M+H]⁺.

### Step c): Preparation of tert-butyl(1-(5-bromo-6-(4-cyano-3-fluorophenyl)-4-methyl-3-nitropyridin-2-yl)piperidin-4-yl)c arbamate

Tert-butyl(1-(5-bromo-6-(4-cyano-3-fluorophenyl)-4-methyl-3-nitropyridin-2-yl)piperidin-4-yl)c arbamate (598 mg, 1.3 mmol) and DMF (2 mL) are added to a reaction flask, added with NBS in batches (347 mg, 1.9 mmol) under ice bath agitation, and reacted while stirring at room temperature for 30 min. When the reaction is completed, the reaction solution is added with water (40 mL), and extracted with ethyl acetate (40 mL×3); organic phase is combined, and washed with saturated saline (40 mL×2), and concentrated under a reduced pressure to dryness; and the residue is purified by silica gel chromatography (eluent: petroleum ether/ethyl acetate=8/3) to obtain tert-butyl(1-(5-bromo-6-(4-cyano-3-fluorophenyl)-4-methyl-3-nitropyridin-2-yl)piperidin-4-yl)c arbamate with a yield of 77.0%.

ESI-MS(m/z)=534.1[M+H]⁺.

### Step d): Preparation of tert-butyl(1-(3-amino-5-bromo-6-(4-cyano-3-fluorophenyl)-4-methylpyridin-2-yl)piperidin-4-yl) carbamate

Tert-butyl(1-(3-amino-5-bromo-6-(4-cyano-3-fluorophenyl)-4-methylpyridin-2-yl)piperidin-4-yl) carbamate (534 mg, 1.0 mmol) and acetic acid (5 mL) are added to a reaction flask, added with zinc powder (300 mg, 5.0 mmol) under ice bath, warmed to 40° C. and reacted for 4 h. When LC-MS monitors that raw materials have been reacted completely, the reaction solution is cooled to room temperature, then quenched with a 1N sodium hydroxide aqueous solution (20 mL), and extracted with ethyl acetate (40 mL×2); organic phase is combined, washed with saturated saline (30 mL×2), dried with anhydrous sodium sulfate, filtered and concentrated under a reduced pressure; and the residue is purified by silica gel chromatography (eluent: petroleum ether/ethyl acetate=1/2) to obtain tert-butyl(1-(3-amino-5-bromo-6-(4-cyano-3-fluorophenyl)-4-methylpyridin-2-yl)piperidin-4-yl) carbamate with a yield of 62.3%.

ESI-MS(m/z)=504.1[M+H]⁺.

### Step e): Preparation of tert-butyl(1-(4-bromo-5-(4-cyano-3-fluorophenyl)-1H-pyrazolo[3,4-c]pyridin-7-yl)piperidin-4-yl) carbamate

Tert-butyl(1-(3-amino-5-bromo-6-(4-cyano-3-fluorophenyl)-4-methylpyridin-2-yl)piperidin-4-yl) carbamate (312 mg, 0.62 mmol), sodium nitrite (43 mg, 0.62 mmol), and acetic acid (5 mL) are added to a reaction flask and reacted at room temperature for 16 h while stirring. When the reaction is completed, the reaction solution is concentrated under a reduced pressure to dryness; the residue is added with sodium bicarbonate water (40 mL) and extracted with ethyl acetate (40 mL×3); organic phase is combined, and washed with saturated saline (40 mL×2), and concentrated under a reduced pressure to dryness; and the residue is purified by silica gel chromatography (eluent: petroleum ether/ethyl acetate=1/2) to obtain tert-butyl(1-(4-bromo-5-(4-cyano-3-fluorophenyl)-1H-pyrazolo[3,4-c]pyridin-7-yl)piperidin-4-yl) carbamate with a yield of 45.0%.

ESI-MS(m/z)=515.1[M+H]⁺.

### Step f): Preparation of tert-butyl(1-(5-(4-cyano-3-fluorophenyl)-4-(3-hydroxy-4-methoxyphenyl)-1H-pyrazole[3,4-c]py ridin-7-yl)piperidin-4-yl)carbamate

Tert-butyl(1-(4-bromo-5-(4-cyano-3-fluorophenyl)-1H-pyrazole[3,4-c]pyridin-7-yl)piperidin-4-y 1)carbamate (144 mg, 0.28 mmol), (3-(benzyloxy)-4-methoxyphenyl)boronic acid (71 mg, 0.42 mmol), Cs₂CO₃ (183 mg, 0.56 mmol), Pd(dppf)Cl₂ (22 mg, 0.03 mmol), 1.4-Dioxane (10 mL) and H₂O (2.5 mL) are added to a reaction flask, and reacted at 120° C. for 1 h while stirring. The reaction solution is concentrated under a reduced pressure to dryness; and the residue is purified by silica gel chromatography (eluent: petroleum ether/ethyl acetate=2/3) to obtain tert-butyl(1-(5-(4-cyano-3-fluorophenyl)-4-(3-hydroxy-4-methoxyphenyl)-1H-pyrazole[3,4-c]py ridin-7-yl)piperidin-4-yl)carbamate with a yield of 56.0%.

ESI-MS(m/z)=559.2[M+H]⁺.

### Step g): Preparation of 4-(7-(4-aminopiperidin-1-yl)-4-(3-hydroxy-4-methoxyphenyl)-1H-pyrazole[3,4-c]pyridin-5-yl)-2-fluorobenzonitrile

Tert-butyl(1-(5-(4-cyano-3-fluorophenyl)-4-(3-hydroxy-4-methoxyphenyl)-1H-pyrazole[3,4-c]py ridin-7-yl)piperidin-4-yl)carbamate (88 mg, 0.16 mmol) is added to a reaction flask, then added with a hydrogen chloride ethyl acetate solution (4M, 2.5 mL) and stirred at room temperature for 1 h; a large amount of solids precipitate, and then concentrated under a reduced pressure to obtain a crude product; and the crude product is purified by Prep-HPLC (separation method 1) to obtain 4-(7-(4-aminopiperidin-1-yl)-4-(3-hydroxy-4-methoxyphenyl)-1H-pyrazolo[3,4-c]pyridin-5-yl)-2-fluorobenzonitrile hydrochloride with a yield of 22.5%.

¹H NMR (400 MHz, DMSO-d₆+D₂0) δ ppm 8.14 (s, 1H), 7.79 (t, J=7.6 Hz, 1H), 7.51 (d, J=10.8 Hz, 1H), 7.32 (dd, J=8.8, 1.4 Hz, 1H), 6.94 (d, J=8.2 Hz, 1H), 6.65 (d, J=7.6 Hz, 2H), 4.88 (d, J=17.6 Hz, 2H), 3.79 (s, 3H), 3.43 (tt, J=10.8, 4.2 Hz, 1H), 3.29 (t, J=12.8 Hz, 2H), 2.09 (q, J=4.4, 3.8 Hz, 2H), 1.71 (qd, J=12.4, 4.0 Hz, 2H).

ESI-MS(m/z)=459.2 [M+H]⁺.

### Example 288

Compound in Example 288 is prepared according to the synthesis method of Example 287 (separation method 1), and its structure and characterization data are as follows:

### 4-(7-(4-aminopiperidin-1-yl)-4-(5-fluoro-3-methylbenzo[d]isoxazol-6-yl)-1H-pyrazole[3,4-c]pyr idin-5-yl)-2-fluorobenzonitrile hydrochloride

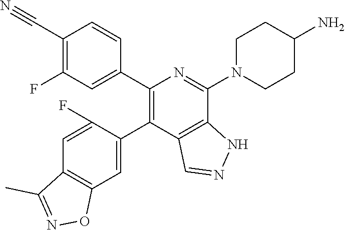 [SMILES: Cc1noc2cc(-c3c(-c4ccc(C#N)c(F)c4)nc(N4CCC(N)CC4)c4[nH]ncc34)c(F)cc12]

¹H NMR (400 MHz, DMSO-d₆) δ ppm 8.23 (s, 1H), 8.08 (m, 3H), 7.92-7.66 (m, 3H), 7.52 (dd, J=10.8, 1.4 Hz, 1H), 7.23 (dd, J=8.0, 1.6 Hz, 1H), 4.37 (s, 2H), 3.47 (s, 2H), 3.22 (t, J=12.8 Hz, 2H), 2.57 (s, 3H), 2.08 (d, J=12.4 Hz, 2H), 1.67 (t, J=11.8 Hz, 2H).

ESI-MS(m/z)=486.2 [M+H]⁺.

### Biological Activity Test

### 1.1 LSD1 Enzyme Activity Detection Method:

The effects of compounds on LSD1 enzyme activity are detected by an HTRF technology to evaluate their inhibition levels on LSD1 enzyme activity. First, 90PM test compound mother solution (dissolved in DMSO) is diluted for a 5-fold concentration gradient with DMSO successively to obtain a total of 8 concentrations of compound working solution 1 (90×). Then, 8 concentrations of working solution 1 are diluted for a 30-fold concentration gradient, i.e, 2 μL of working solution 1 is sucked and added to 58 μL of Buffer, and fully shaken and mixed well on a vortex mixer to obtain a total of 8 concentrations of screening compound working solution 2 (3×). In a 384-well shallow white plate, 2 μL of 3×LSD1 (Activemotif, 31426) enzyme solution and 2 μL of compound working solution 2 (3×) are sequentially added to each well, mixed well, and incubated at room temperature for 15 min; 2 μL of 3×H3K4mel (Anaspec, AS-64355-025) substrate solution is sequentially added to each well, mixed well, and incubated at room temperature for 60 min; 2 μL of stop solution (containing 5.4 mM 2-PCPA) is sequentially added to each well, mixed well, and incubated for 15 min at room temperature; and 4 μL of Eu-anti H3K4 (PerkinElmer, TRF0404-D) and allophycocyanin (Prozyme, PJ27S) premixed antibody (1:1) solution is sequentially added to each well, mixed well, and incubated at room temperature for 60 min. The 384-well plate is placed on a multifunctional microplate reader for reading values, an excitation light wavelength is set to 337 nm, and values at 620 nm and 665 nm are recorded. The data results are presented as a ratio of a 665 nm signal value to a 620 nm signal value per well, that is: Ratio=104×665 nm signal value/620 nm signal value. An inhibition rate is calculated by the following formula:

% Inhibition rate=[(negative-test compound)/(negative-Blank)]*100

IC₅₀ is fitting calculated from the inhibition rate by selecting log(inhibitor) vs. response-Variable Slope(four parameters) by GraphPad Prim software.

Note: Negative is an inhibitor-free group; Blank is an enzyme-free group.

### 1.2 In Vitro Cell Proliferation Detection Method for Screened Compound

The number of viable cells is measured by a CellTiter-Glo® reagent to assess the inhibitory effect of the compound on cell proliferation. NCI-H1417 cells in the logarithmic growth phase are collected and inoculated into a 96-well plate with a transparent bottom at 100 μl per well and a density of 7×10′ cells/well, and cultured overnight at 37° C., 5% CO₂; the compound is diluted by 5-fold successively with DMSO to obtain gradient diluents of 8 concentrations, and then diluted with BEGM (100 FBS) cell culture medium to obtain a compound working solution (2×), which is added to a cell supernatant at 100 μL per well, and continued to be cultured for 7 days at 37° C. and 500 CO₂. The plate is removed, and the plate and CellTiter-Glo® mixed reagent are equilibrated for about 10-30 min at room temperature (25° C.); and after centrifugation, 100 μL of medium is carefully pipetted, and 85 μL of CellTiter-Glo® reagent is then added. Cells are well mixed with the CellTiter-Glo® mixed reagent for 2 min using a microwell shaker and incubated for 10 min at room temperature. The 96-well plate is placed on a multifunctional microplate reader to record a luminescence value (RLU).

An inhibition rate is calculated by the following formula:

$$\% inhibition rate = [ \frac{{Ratio}_{Negative control} - {Ratio}_{Test compound}}{{Ratio}_{Negative control}} ] * 100$$

IC₅₀ is calculated from the inhibition rate by GraphPad Prism software.

Note: Negative control is an inhibitor-free group.

TABLE I
Compound activity

| | Inhibition of | NCI-H1417 |
|---|---|---|
| | enzymatic | proliferation |
| | activity of LSD1 | inhibition |
| Examples | IC50(nM) | IC50(nM) |
| 1 | 186.3 ± 69.6 | 11.3 |
| 2 | 17.9 ± 3.2 | 0.73 |
| 3 | 0.80 ± 0.08 | 34.2 |
| 4 | 2.30 ± 0.74 | 30.4 |
| 5 | 28.5 ± 1.9 | 165 |
| 6 | 0.30 ± 0.25 | 3.64 |
| 7 | 0.40 ± 0.29 | 46.4 |
| 8 | 0.30 ± 0.04 | 6.94 |
| 9 | 0.026 ± 0.014 | 0.52 |
| 10 | 79.1 ± 18.9 | 371 |
| 11 | 2.90 ± 0.28 | 13.03 |
| 12 | 42.4 ± 4.1 | 1.71 |
| 14 | 13.8 | 64.9 |
| 15 | 49.9 | 103.8 |
| 16 | 15.2 ± 3.7 | 38.05 |
| 17 | 19.39 | >1000 |
| 18 | 78.33 | 87.15 |
| 19 | 5.965 | >1000 |
| 20 | 166.2 | N/A |
| 21 | 10.48 | 38.71 |
| 22 | 9.026 | 34.94 |
| 23 | 0.18 ± 0.08 | 0.4397 |
| 24 | 21.47 ± 20.46 | 252 |
| 25 | 71.7 | 868 |
| 26 | 3.67 ± 0.72 | 7.45 |
| 27 | 77.5 | 446 |
| 28 | 441 | N/A |
| 29 | 3.38 | 11.8 |
| 30 | 213.3 | N/A |
| 31 | 51 | 77.2 |
| 32 | 48.8 | 262 |
| 33 | 210.12 | N/A |
| 35 | 35.7 | 31.8 |
| 36 | 35.2 | 21.2 |
| 37 | 15.1 | 173 |
| 38 | 57.6 | 179 |
| 39 | 21.5 ± 7.6 | 121.3 |
| 40 | 280 | N/A |
| 41 | 22.1 | 7.93 |
| 42 | 1.0 ± 0.1 | 8.78 |
| 44 | 12.8 ± 4.6 | 28.9 |
| 45 | 184 | 160.5 |
| 46 | 2.0 ± 1.4 | 96.4 |
| 47 | 2.6 ± 1.3 | 105.8 |
| 48 | 57.8 | 290 |
| 49 | 61.4 | 227.8 |
| 50 | 7.0 ± 2.1 | 33.2 |
| 51 | 64 ± 20 | 88.2 |
| 52 | 100 | 167 |
| 55 | 2.3 ± 0.7 | 10 ± 2.9 |
| 56 | 2.9 ± 1.2 | 4.88 |
| 58 | 1.3 ± 0.4 | 7.26 |
| 59 | 0.7 ± 0.3 | 2.35 |
| 60 | 0.4 ± 0.2 | 0.49 |
| 61 | 26.45 | 386.7 |
| 62 | 21.0 | 223.7 |
| 63 | 5.1 ± 0.4 | 191.5 |
| 64 | 119.3 | 344 |
| 65 | 0.7 ± 0.4 | 0.95 |
| 66 | 0.2 ± 0.1 | 0.2 |
| 67 | 0.2 ± 0.1 | 0.2 |
| 68 | 3.4 ± 1.3 | 6.92 |
| 69 | 3.9 ± 2.7 | 8.41 |
| 70 | 0.5 ± 0.3 | 0.885 |
| 71 | 0.3 ± 0.2 | 0.338 |
| 72 | 6.6 ± 1.0 | 12.2 |
| 73 | 1.9 ± 0.4 | 1.61 |
| 74 | 166.3 | N/A |
| 75 | 499.6 | N/A |
| 76 | 4.9 ± 2.5 | 1.37 |
| 77 | 2.4 ± 0.9 | 2.19 |
| 78 | 1.6 ± 0.3 | 2.53 |
| 79 | 0.3 ± 0.1 | 0.272 |
| 80 | 1.0 ± 0.4 | 1.22 |
| 81 | 671 | N/A |
| 82 | 12.9 ± 0.2 | 72.4 |
| 83 | 35.4 | 629.9 |
| 84 | 2.18 | 4.91 |
| 85 | 0.6 | 0.76 |
| 86 | 0.147 | 0.63 |
| 87 | 2.82 ± 1.49 | 3.287 |
| 88 | 565.9 | N/A |
| 89 | 0.46 ± 0.11 | 1.003 |
| 90 | 7.48 | 5.34 |
| 91 | 1.1 ± 0.3 | 1.82 |
| 92 | 8.2 ± 0.1 | 8.78 |
| 93 | 153 | 30.7 |
| 94 | 24.9 | 103.6 |
| 95 | 0.16 ± 0.11 | 0.467 |
| 96 | 3.0 ± 0.6 | 11.8 |
| 97 | 88.45 | 185 |
| 98 | 0.70 ± 0.0.2 | 0.604 |
| 99 | 4.9 ± 1.2 | 1.72 |
| 100 | 8.9 ± 0.3 | 3.18 |
| 101 | 1.0 ± 0.2 | 0.917 |
| 102 | 712 | N/A |
| 103 | 0.6 ± 0.2 | 6.27 |
| 104 | 0.7 ± 0.02 | 1.76 |
| 105 | 2.3 ± 0.04 | 5.45 |
| 106 | 1.2 ± 0.7 | 0.594 |
| 107 | 2.5 ± 1.7 | 8.35 |
| 108 | 1.5 ± 0.7 | 4.02 |
| 109 | 4.0 ± 1.7 | 15.98 |
| 110 | 3.20 ± 0.37 | 42.6 |
| 114 | 67 | >1000 |
| 117 | 0.46 ± 0.02 | 3.76 |
| 118 | 0.27 ± 0.01 | 0.6 |
| 119 | 9.54 ± 1.2 | 39.06 |
| 120 | 0.77 ± 0.43 | 2.081 |
| 121 | 3.15 ± 1.03 | 8.381 |
| 122 | 8.68 ± 0.76 | 30.16 |
| 123 | 28.7 | 47.65 |
| 124 | 0.08 ± 0.03 | 0.163 |
| 125 | 0.35 ± 0.09 | 0.386 |
| 126 | 15.9 | 21.3 |
| 127 | 34.4 | 135 |
| 128 | 4.19 ± 0.11 | 6.46 |
| 129 | 0.61 ± 0.13 | 111.6 |
| 130 | 10.74 | 17.6 |
| 131 | 4.46 ± 0.09 | 80.7 |
| 132 | 5.62 | 11.9 |
| 133 | 0.54 ± 0.32 | 1.04 |
| 134 | 6.1 ± 1.9 | 7.25 |
| 135 | 9.6 ± 2.7 | 8.19 |
| 136 | 0.06 ± 0.02 | 0.176 |
| 137 | 0.07 ± 0.05 | 0.553 |
| 138 | 0.1 ± 0.05 | 0.305 |
| 139 | 0.69 ± 0.18 | 2.83 |
| 140 | 0.4 ± 0.1 | 0.20 ± 0.15 |
| 141 | 224 | N/A |
| 142 | 0.198 | 0.214 |
| 143 | 5.5 ± 2.9 | 4.82 |
| 144 | 1.6 ± 0.2 | 18.2 |
| 145 | 1.0 ± 0.2 | 2.42 |
| 146 | 0.5 ± 0.2 | 8.90 |
| 147 | 11.5 ± 4.4 | 8.00 |
| 148 | 6.9 ± 1.3 | 78.0 |
| 149 | 33.2 | 12.8 |
| 150 | 20.3 | 31.2 |
| 152 | 23.5 | 12.4 |
| 153 | 5.2 ± 0.5 | 31.6 |
| 154 | 0.5 ± 0.1 | 0.959 |
| 155 | 0.2 ± 0.1 | 0.352 |
| 156 | 0.2 ± 0.06 | 0.447 |
| 157 | 0.30 ± 0.3 | 2.05 |
| 158 | 0.2 ± 0.2 | 0.763 |
| 159 | 2.8 ± 1.0 | 5.82 |
| 160 | 1.2 ± 0.2 | 0.29 |
| 161 | 3.9 ± 0.2 | 8.37 |
| 162 | 59.9 | 11.65 |
| 163 | 5.6 ± 1.6 | 9.11 |
| 164 | 0.4 ± 0.2 | 0.22 |
| 165 | 3.4 ± 1.5 | 4.68 |
| 166 | 1.0 ± 0.5 | 2.39 |
| 167 | 78.4 | 155.2 |
| 168 | 0.5 ± 0.1 | 1.24 |
| 169 | 4.3 ± 0.9 | 4.0 |
| 170 | 13.2 | 18.3 |
| 171 | 1.9 ± 1.5 | 6.0 |
| 172 | 5.0 ± 4.1 | 149.0 |
| 173 | 0.8 ± 0.2 | 8.15 |
| 174 | 13.2 | 14.4 |
| 175 | 3.2 ± 2.1 | 4.13 |
| 176 | 16.8 | 21.6 |
| 178 | 3.8 ± 2.2 | 6.24 |
| 179 | 938.5 | N/A |
| 180 | 77.1 | N/A |
| 181 | 192.6 | N/A |
| 183 | 3.6 ± 0.6 | 6.28 |
| 184 | 110.5 | N/A |
| 185 | 14.1 | 159.6 |
| 186 | 17.3 | 18.0 |
| 187 | 0.8 ± 0.1 | 1.11 |
| 188 | 35 | >1000 |
| 189 | 0.29 ± 0.11 | 4.04 |
| 190 | 0.331 | 1122 |
| 191 | 104 | N/A |
| 192 | 10.6 | 81 |
| 193 | 1.72 | 61.4 |
| 195 | 208 | N/A |
| 196 | 37.7 | 656 |
| 197 | 9.44 ± 5.96 | 24.18 |
| 198 | 174.4 | N/A |
| 199 | 72 | >1000 |
| 200 | 0.69 | 34 |
| 201 | 26.8 | 384 |
| 202 | 77.1 | >1000 |
| 203 | 0.39 | 37.6 |
| 204 | 12.2 ± 7.3 | 118 |
| 205 | 902 | N/A |
| 206 | 432 | N/A |
| 207 | 1254 | N/A |
| 208 | 641 | N/A |
| 209 | 0.507 | 1.44 |
| 210 | 5524 | N/A |
| 211 | 206 | N/A |
| 212 | 207.1 | N/A |
| 213 | 36.7 | N/A |
| 214 | 0.47 ± 0.13 | 493.3 |
| 215 | 0.45 ± 0.05 | 4.19 |
| 216 | 8.14 ± 1.84 | 20.86 |
| 217 | 12.8 | 67.8 |
| 218 | 0.15 ± 0.03 | 0.88 |
| 219 | 7.30 ± 0.46 | 95.3 |
| 220 | 0.42 ± 0.06 | 1.32 |
| 221 | 10.0 ± 3.2 | 29.8 |
| 222 | 14.39 | 41 |
| 223 | 0.1 ± 0.03 | 2.39 |
| 224 | 25.7 | 11.6 |
| 225 | 52.1 | 27.8 |
| 226 | 19.8 | 2.53 |
| 227 | 42.6 | 3.91 |
| 228 | 22.9 | 4.68 |
| 229 | 12.99 | 2.05 |
| 230 | 47.9 | 3.92 |
| 231 | 22.5 | 30.2 |
| 232 | 548.5 | 209.3 |
| 233 | 7.2 ± 2.3 | 9.36 |
| 234 | 165 ± 8 | 31 ± 8 |
| 235 | 284.7 | 98.3 |
| 236 | 21.7 | 2.82 |
| 237 | 423 ± 23 | 28.3 |
| 238 | 98 ± 1 | 5.31 |
| 239 | 4.8 ± 1.8 | 0.986 |
| 240 | 770 | 29.5 |
| 241 | 26.9 | 12.6 |
| 242 | 49.6 | 2.83 |
| 244 | 116.1 | 5.07 |
| 245 | 39.1 | 1.98 |
| 246 | 18.1 | 1.782 |
| 247 | 65.8 | 6.51 |
| 248 | 139.7 | 2.98 |
| 249 | 15.8 | 1.11 |
| 251 | N/A | 599.5 |
| 252 | 73.7 | >1000 |
| 254 | 121 ± 58 | N/A |
| 255 | 171.5 | 29.1 |
| 256 | 71.3 | 10.8 |
| 257 | 43.1 ± 25.4 | 2.3 |
| 258 | 232.4 | 89.72 |
| 259 | 139.1 | 76.6 |
| 260 | 156 | 46.1 |
| 261 | 53.5 | 3.22 |
| 262 | 157 ± 40 | 16.2 |
| 263 | 17.2 | 6.90 |
| 264 | 87.6 | 27.4 |
| 265 | 224.2 | N/A |
| 266 | 0.7 ± 0.004 | 0.48 |
| 267 | 1.0 ± 0.4 | 3.87 |
| 268 | 1.5 ± 0.5 | 0.378 |
| 269 | N/A | 232.7 |
| 270 | 10.1 | 8.64 |
| 271 | 0.5 ± 0.4 | 9.77 |
| 272 | 1.8 ± 0.8 | 11.0 |
| 273 | 0.2 ± 0.05 | 1.19 |
| 274 | 23.76 | >1000 |
| 275 | 0.94 | 3.6 |
| 276 | 0.1 ± 0.01 | 1.1 ± 0.7 |
| 277 | 0.39 | 1.33 |
| 278 | 0.3 ± 0.1 | 0.648 |
| 279 | 2.27 | 6.61 |
| 280 | 0.272 | 2.7 ± 1.0 |
| 281 | 2.23 | 14.8 |
| 282 | N/A | 136 |
| 284 | 633.4 | N/A |
| 285 | 0.93 | 2.16 |
| 286 | 3.5 | 4.58 |
| 287 | 0.265 | 5.97 |
| 288 | 0.591 | 6.33 |
| Reference compound | 2.1 ± 1.5 | 5.0 ± 2.0 |
| (CC90011) | | |

In the table above, N/A means not applicable.
Conclusion: The compounds of the present invention have significant inhibitory effects on LSD1.

### 2. CYP 450 Enzyme Inhibition Test

Using mixed human liver microsomes as a CYP 450 enzyme source, specific probe substrates of respective CYP isoenzymes (2 substrates for CYP 3A4) are incubated respectively with test compounds of a series of concentrations in the presence of cofactor NADPH. LC-MS/MS is used to determine the amount of metabolites generated by a probe substrate in the incubation system, the IC₅₀ values of the test compounds on specific isoenzymes/substrates are calculated, and their inhibitory effects on the activities of human liver microsomal cytochrome P450 isoenzymes are evaluated. During the experiment, 49 μl of PBS, 50 μl of probe substrate, and 50 μl of human liver microsomal working solution are sequentially added to the incubation system, then added with 1 μl of the test compound working solutions of various concentrations and mixed well. After 5 min of pre-incubation at 37° C., 50 μl of NADPH is added to start the reaction. After incubation for the corresponding time, an appropriate amount of glacial acetonitrile containing an internal standard is added to terminate the reaction, vortexed and mixed well, and centrifuged to take the supernatant, and the supernatant is injected into LC-MS/MS to detect the amount of metabolites generated by the probe substrate. The percentages of residual enzyme activity of metabolites at different test compound concentrations are calculated with the enzyme activity at 0 concentration point (characterized by the amount of metabolites generated) being 100%. IC₅₀ is calculated from the residual enzyme activity by GraphPad Prism software.

TABLE II
CYP inhibition of representative compounds

| Examples | CYP3A4-T IC50 (μM) |
|---|---|
| 12 | 5.59 |
| 23 | 54.2 |
| 66 | 3.65 |
| 70 | 8.71 |
| 71 | 4.3 |
| 73 | 7.59 |
| 79 | >10 |
| 80 | >10 |
| 87 | 6.74 |
| 118 | 8.68 |
| 120 | 8.93 |
| 125 | 49.9 |
| 128 | >50 |
| 155 | 18.0 |
| 164 | >10 |
| 169 | >10 |
| 175 | 8.08 |
| 224 | 5.54 |
| 227 | 14.1 |
| 239 | >10 |
| 273 | >10 |
| 280 | >10 |
| Reference compound | >50 |

3. hERG Inhibition Test

A manual patch-clamp technology is used to evaluate whether the test compound has a potential inhibitory effect on a voltage-gated potassium ion channel hERG. In this experiment, the effects of 5 concentrations of compound (2 parallel samples per concentration) on the current of the hERG channel are detected, a dose-response curve of the compound is obtained and IC₅₀ is calculated. First, the hERG current measured in the extracellular fluid containing 0.1% DMSO is used as a baseline for detection. After the hERG current remains stable for at least 5 min, the solution containing the test compound is perfused sequentially around the cells from low to high concentration. It is necessary to wait about 5 min after each perfusion to allow the compound to act fully on the cells and record the hERG current synchronously. After the recorded current tends to stabilize, the last five hERG current values are recorded, and averaged as a final current value at a specific concentration. After testing the compound, 150 nM Dofetilide is added to the same cell to completely inhibit its current as a positive control for this cell. At the same time, the positive compound Dofetilide is simultaneously detected with the same patch-clamp system before and after the end of a test drug experiment to ensure the reliability and sensitivity of the entire detection system. The data that meets the acceptance criteria is outputted by PatchMaster software, and the current inhibition percentage is calculated using the following formula.

$$Tail current inhibition rate = \frac{Tail current {value}_{compound -} Tail current {value}_{positive drug}}{Tail current {value}_{blank -} Tail current {value}_{positive drug}} ) \times 100$$

The dose-effect curve is fitted by Graphpad Prism 8.0 software and the IC₅₀ value is calculated.

TABLE III
hERG inhibition of representative compounds

| Examples | hERG IC₅₀ (μM) |
|---|---|
| 2 | 2.58 |
| 9 | 2.10 |
| 11 | >30 |
| 12 | 4.69 |
| 23 | >30 |
| 26 | >30 |
| 41 | >30 |
| 42 | >30 |
| 50 | 17.64 |
| 66 | 15.46 |
| 79 | 4.38 |
| 80 | 9.33 |
| 89 | 13.84 |
| 90 | 4.62 |
| 92 | 10.92 |
| 95 | 6.92 |
| 98 | 5.85 |
| 104 | 6.70 |
| 105 | 5.60 |
| 117 | 7.19 |
| 118 | 13.24 |
| 120 | 7.83 |
| 121 | 6.04 |
| 124 | >30 |
| 125 | 9.52 |
| 128 | 13.66 |
| 132 | >30 |
| 133 | 7.17 |
| 134 | 18.39 |
| 135 | 23.41 |
| 136 | >30 |
| 137 | >10 |
| 215 | 11.39 |
| 224 | 5.60 |
| 227 | 6.29 |
| 228 | 7.21 |
| 230 | 4.99 |
| 233 | 4.47 |
| 236 | 5.40 |
| 246 | 8.55 |
| 257 | 2.83 |
| Reference compound | 17.14 |

### 4. Caco-2 Permeability Test

A Caco-2 cell model is used to evaluate the permeability of the test compound. In this experiment, Caco-2 cells are incubated with the test compound at 37° C., 500 CO₂, 9500 relative humidity, the compound concentrations on both sides of AB are determined by LC-MS/MS, and the apparent permeability coefficient and efflux ratio are calculated. During the experiment, Caco-2 cells are inoculated into a Transwell chamber, exchanged for mediums every two days, and cultured to 21-28 days; the transmembrane resistance value of the cell membrane is determined, the integrity of the cell membrane is evaluated, and the permeability test may only be carried out after the membrane resistance reaches the qualified standard. According to different test groups, a compound containing permeate or a compound free permeate is respectively added on side A and B, and incubated in an incubator at 37° C., 500 CO₂, 9500 relative humidity for 120 min, and samples are taken after the test for LC-MS/MS detection. In addition, after the permeability test, the fluorescent yellow transmittance of Caco-2 cells should be measured to further evaluate the integrity of the monolayer membrane. After all tests are completed, the apparent permeability coefficient and efflux ratio of the test compound are calculated according to the following formula.

$${P}_{a p p} = \frac{{V}_{A}}{Area \times time} \times \frac{{[ drug ]}_{acceptor}}{{[ drug ]}_{initial donor}} )$$

$$Efflux ratio = \frac{{P}_{a p p ( B - A )}}{{P}_{a p p ( B - A )}}$$

TABLE IV
Caco-2 membrane permeability of representative compounds

| | Mean Papp (10⁻⁶ cm/s) | | |
|---|---|---|---|
| Examples | A to B | B to A | Efflux Ratio |
| 2 | 14.97 | 15.03 | 1.00 |
| 9 | 1.50 | 37.63 | 25.20 |
| 11 | 3.66 | 27.17 | 7.42 |
| 12 | 5.95 | 6.92 | 1.16 |
| 87 | 2.97 | 3.35 | 1.13 |
| 120 | 2.88 | 7.37 | 2.56 |
| 124 | 1.73 | 24.73 | 14.27 |
| 125 | 4.27 | 24.97 | 5.84 |
| 220 | 2.30 | 6.21 | 2.70 |
| 236 | 1.44 | 3.99 | 2.76 |
| 246 | 3.59 | 3.36 | 0.96 |
| 257 | 1.54 | 4.55 | 2.95 |
| 261 | 0.49 | 0.67 | 1.38 |
| Reference compound | 7.26 | 41.00 | 5.66 |

### 5. Rat PK Test

Male SD rats are administered the test compound by intravenous injection (IV) and gavage (PO), respectively and the plasma concentrations of the test compounds in rats are measured by LC-MS/MS, the main pharmacokinetic parameters are calculated, and the pharmacokinetic behaviors in vivo are evaluated. In the experiment, whole blood is collected before IV administration and 0.083 h, 0.25 h, 0.5 h, 1 h, 2 h, 4 h, 6 h, 8 h and 24 h after IV administration; and before PO administration and 0.25 h, 0.5 h, 1 h, 2 h, 4 h, 6 h, 8 h and 24 h after PO administration, respectively. The whole blood is placed in K₂-EDTA anticoagulant tubes, centrifuged (4° C.) within 10 min to separate plasma, and stored at −80° C. for testing. During sample processing, an appropriate amount of methanol or acetonitrile containing internal standards is used for protein precipitation, vortexing, and centrifugation, and then the supernatant is injected into LC-MSMS for detection. A non-atrioventricular model of WinNonLin 8.3 is used to calculate pharmacokinetic parameters.

TABLE V
Rat PK of representative compounds

| | Cl(ml/min/ | Cₘₐₓ(ng/ | | | | |
|---|---|---|---|---|---|---|
| Examples | kg/ml) | ml) | Vss(l/kg) | T_{1/2}(h) | AUC_{INF}(h*ng/ml) | F(%) |
| 66 | 68.2 | 42.5 | 11.3 | 1.96 | 227 | 17.7 |
| 89 | 58.3 | 22.5 | 13.0 | 6.26 | 208 | 13.1 |
| 118 | 56.1 | 26.9 | 20.0 | 4.67 | 192 | 12.1 |
| 120 | 39.9 | 34.7 | 14.5 | 4.82 | 310 | 14.4 |
| 125 | 60.6 | 65.0 | 14.7 | 2.58 | 323 | 21.8 |
| 246 | 39.6 | 56.2 | 4.22 | 4.96 | 265 | 9.54 |
| Reference | 94.5 | 26.0 | 19.1 | 2.32 | 132 | 14.3 |
| compound | | | | | | |

The reference compound is CC-90011:

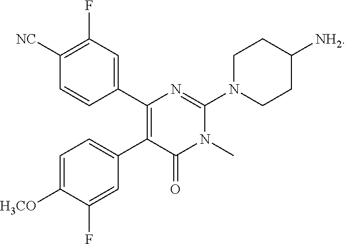 [SMILES: COc1ccc(-c2c(-c3ccc(C#N)c(F)c3)nc(N3CCC(N)CC3)n(C)c2=O)cc1F]

What is claimed is:

1. A compound of formula (I), or a tautomer, mesomer, racemate, enantiomer, diastereoisomer, or mixture thereof, or a deuterated isotopic derivative, pharmaceutically acceptable hydrate, solvate, salt or cocrystal thereof, 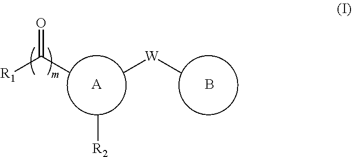
wherein
ring A is selected from C₆₋₁₀ aryl, C₃₋₁₀ heteroaryl, C₃₋₁₀ cycloalkyl, C₂₋₁₀ heterocycloalkyl, C₃₋₁₀ cycloalkenyl, C₂₋₁₀ heterocycloalkenyl, C₆₋₁₀ aryl fused C₃₋₁₀ cycloalkyl, C₆₋₁₀ aryl fused C₂₋₁₀ heterocycloalkyl, C₆₋₁₀ aryl fused C₃₋₁₀ cycloalkenyl, C₆₋₁₀ aryl fused C₂₋₁₀ heterocycloalkenyl, 5-10-membered heteroaryl fused C₃₋₁₀ cycloalkyl, 5-10-membered heteroaryl fused C₂₋₁₀ heterocycloalkyl, 5-10-membered heteroaryl fused C₃₋₁₀ cycloalkenyl, and 5-10-membered heteroaryl fused C₂₋₁₀ heterocycloalkenyl; the above groups are optionally substituted by 0 to 4 substituents selected from halogen, —CN, hydroxy, C₁₋₆ alkyl, halogen substituted C₁₋₆ alkyl, C₁₋₆ alkoxy, halogen substituted C₁₋₆ alkoxy, C₆₋₁₀ aryl substituted C₁₋₆ alkoxy, —OC₃₋₁₀ cycloalkyl, C₃₋₁₀ cycloalkyl, —O—C₁₋₆ alkyl-(C₃₋₁₀ cycloalkyl), —O—(C₂₋₁₀ heterocycloalkyl), C₂₋₁₀ heterocycloalkyl, —O—C₁₋₆ alkyl-(C₂₋₁₀ heterocycloalkyl), C₂₋₆ alkenyl, C₂₋₆ alkynyl, ═O, —NR_{c}R_{d}, and 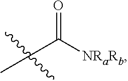
and the ═O can only be a substituent of a non-aromatic ring; and the heteroaryl, heterocycloalkyl, heterocycloalkenyl, C₆₋₁₀ aryl fused C₂₋₁₀ heterocycloalkyl, C₆₋₁₀ aryl C₂₋₁₀ fused heterocycloalkenyl, 5-10-membered heteroaryl fused C₃₋₁₀ cycloalkyl, 5-10-membered heteroaryl fused C₂₋₁₀ heterocycloalkyl, 5-10-membered heteroaryl fused C₃₋₁₀ cycloalkenyl, 5-10-membered heteroaryl fused C₂₋₁₀ heterocycloalkenyl each contain 1 to 4 heteroatoms selected from N, O or S; 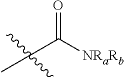 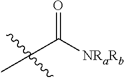
ring B is selected from C₆₋₁₀ aryl, C₃₋₁₀ heteroaryl, C₃₋₁₀ cycloalkyl, and C₂₋₁₀ heterocycloalkyl; the aryl, the heteroaryl, the cycloalkyl and the heterocycloalkyl are optionally substituted by 0 to 4 substituents selected from halogen, —CN, hydroxy, C₁₋₆ alkyl, halogen substituted C₁₋₆ alkoxy, C₃₋₁₀ cycloalkyl, C₂₋₁₀ heterocycloalkyl, C₂₋₆ alkenyl, C₂₋₆ alkynyl, or —NR_{c}R_{d}; the heteroaryl and the heterocycloalkyl each contain 1 to 4 heteroatoms selected from N, O or S; provided that ring B is not a 9-membered spiro ring;
W is selected from a bond, 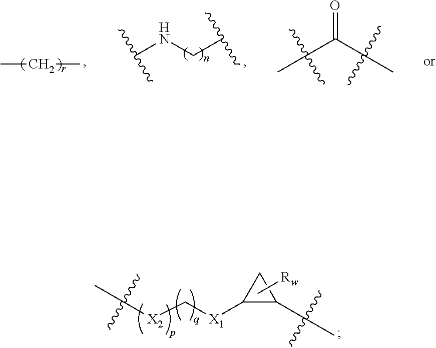
and when W is a bond, ring A and ring B are directly connected via the bond; 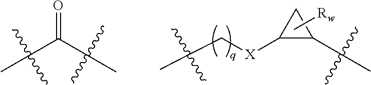
X₁ is selected from —NRₓ—, —O—, or —CHRₓ—;
X₂ is selected from —NH—, or —O—;
R_{W} and Rₓ are independently selected from H, C₁₋₆ alkyl, C₁₋₆ alkoxy, halogen, —CN, C₂₋₆ alkenyl, C₂₋₆ alkynyl, —C(═O)C₁₋₆ alkyl, —OC₃₋₆ cycloalkyl, C₆₋₁₀ aryl, C₅₋₁₀ heteroaryl, C₃₋₁₀ cycloalkyl, and C₂₋₁₀ heterocycloalkyl; the alkyl, the alkenyl, the alkynyl, the aryl, the heteroaryl, the cycloalkyl or heterocycloalkyl are optionally substituted by 0 to 4 substituents selected from halogen, halogen substituted C₁₋₆ alkyl, —CN, hydroxy, C₁₋₆ alkyl, C₁₋₆ alkoxy, —C(═O)C₁₋₆ alkyl, —OC₃₋₆ cycloalkyl or C₁₋₄ alkylthio; the heterocyclyl contains 1 to 3 heteroatoms selected from N, o or S;
R₁ and R₂ are independently selected from hydrogen, C₁₋₆ alkyl, C₁₋₆ alkoxy, halogen, —CN, C₂₋₆ alkenyl, C₂₋₆ alkynyl, —C(═O)C₁₋₆ alkyl, —OC₃₋₆ cycloalkyl, —OC₆₋₁₀ aryl, —O—(5-10-membered heteroaryl), C₆₋₁₀ aryl, C₅₋₁₀ heteroaryl, C₃₋₁₀ cycloalkyl, C₂₋₁₀ heterocycloalkyl, C₃₋₁₀ cycloalkenyl, C₂₋₁₀ heterocycloalkenyl, C₆₋₁₀ aryl fused C₂₋₁₀ heterocycloalkyl, and —NR₄R₅; the alkyl, the alkenyl, the alkynyl, the aryl, the heteroaryl, the cycloalkyl, the heterocycloalkyl, the cycloalkenyl, and the heterocycloalkenyl are optionally further substituted by 0 to 4 substituents selected from halogen, —CN, —CH₂CN, —NH₂, hydroxy, C₁₋₆ alkyl, halogen substituted C₁₋₆ alkyl, hydroxy substituted C₁₋₆ alkyl, C₁₋₆ alkoxy, halogen substituted C₁₋₆ alkoxy, hydroxy substituted C₁₋₆ alkoxy, ═O, —C(═O)C₁₋₆ alkyl, —C(═O)OC₁₋₆ alkyl, —COOH, C₂₋₆ alkynyl, hydroxy substituted C₂₋₆ alkynyl, —OC₃₋₆ cycloalkyl, C₁₋₄ alkylthio, C₅₋₁₀ heteroaryl, C₆₋₁₀ aryl, C₄₋₁₀ cycloalkyl, C₂₋₁₀ heterocycloalkyl, and —S(O)₂Rₐ; the heteroaryl and heterocycloalkyl each contain 1 to 3 heteroatoms selected from N, O or S;
R₄ and R₅ are independently selected from hydrogen, C₁₋₆ alkyl, amino substituted C₁₋₆ alkyl, C₃₋₁₀ cycloalkyl, C₂₋₁₀ heteroaryl, C₆₋₁₀ aryl, and 5-10-membered heteroaryl; and Ro is selected from C₁₋₆ alkyl, and amino;
Rₐ and R_{b} are independently selected from hydrogen, C₁₋₆ alkyl, C₁₋₆ alkoxy, halogen, —CN, C₂₋₆ alkenyl, C₂₋₆ alkynyl, —C(═O)C₁₋₆ alkyl, —OC₃₋₆ cycloalkyl, C₄₋₁₀ cycloalkyl, C₆₋₁₀ aryl, C₅₋₁₀ heteroaryl, C₃₋₁₀ cycloalkyl, and C₂₋₁₀ heterocycloalkyl; the alkyl, the alkenyl, the alkynyl, the aryl, the heteroaryl, the cycloalkyl, or the heterocycloalkyl is optionally further substituted by 0 to 4 substituents selected from halogen, halogen substituted C₁₋₆ alkyl, —CN, hydroxy, C₁₋₆ alkyl, C₁₋₆ alkoxy, —C(═O)C₁₋₆ alkyl, —OC₃₋₆ cycloalkyl, C₁₋₄ alkylthio, C₅₋₁₀ heteroaryl, C₆₋₁₀ aryl, C₄₋₁₀ cycloalkyl, C₂₋₁₀ heterocycloalkyl, and —S(O)₂Rₐ; and the heterocyclyl contains 1 to 3 heteroatoms selected from N, O or S;
R_{c} and Rₐ are independently selected from hydrogen, C₁₋₆ alkyl, C₁₋₆ alkoxy, halogen, —CN, C₂₋₆ alkenyl, C₂₋₆ alkynyl, —C(═O)C₁₋₆, alkyl, —OC₃₋₆, cycloalkyl, C₃₋₁₀ cycloalkyl, C₆₋₁₀ aryl, C₅₋₁₀ heteroaryl, C₄₋₁₀ cycloalkyl, and C₂₋₁₀ heterocycloalkyl; the alkyl, the alkenyl, the alkynyl, the aryl, the heteroaryl, the cycloalkyl, or the heterocycloalkyl is optionally further substituted by 0 to 4 substituents selected from halogen, hydroxy, amino, aminoalkyl, aminocycloalkyl, aminoheterocyclyl, C₁₋₆ alkyl, C₁₋₆ alkoxy, C₅₋₁₀ heteroaryl, aryl, hydroxy, —CN, and —S(O)₂Rₐ;
the heterocyclyl contains 1 to 3 heteroatoms selected from N, O or S; R₆ is selected from C₁₋₆ alkyl, and amino; when ring A is selected from a aromatic heterocycle fused by a 5-membered ring and a 6-membered ring, R_{c} and Rₐ are not simultaneously methyl;
m and n are independently selected from integers from 0 to 6; when m is 0, R₁ is directly connected to ring A; 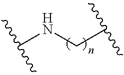
r is an integer selected from 1 to 6;
p is selected from 0 or 1; and when p is 0, X₂ is absent; and
q is selected from 1, 2, 3 or 4.

2-96. (canceled)

97. The compound according to claim 1, wherein the compound is selected from the compound of formula (I-6a), 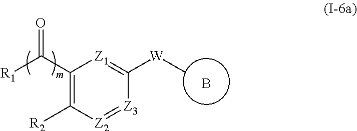
wherein Z₁, Z₂, and Z₃ are each independently selected from N or ═CR_{z}—; and
R_{z} is selected from hydrogen, halogen, —CN, hydroxy, C₁₋₆ alkyl, halogen substituted C₁₋₆ alkyl, C₁₋₆ alkoxy, halogen substituted C₁₋₆ alkoxy, C₆₋₁₀ aryl substituted C₁₋₆ alkoxy, —OC₃₋₁₀ cycloalkyl, C₃₋₁₀ cycloalkyl, —O—C₁₋₆ alkyl-(C₃₋₁₀ cycloalkyl), —O—(C₂₋₁₀ heterocycloalkyl), C₂₋₁₀ heterocycloalkyl, —O—C₁₋₆ alkyl-(C₂₋₁₀ heterocycloalkyl), C₂₋₆ alkenyl, C₂₋₆ alkynyl, —NR_{c}R_{d}, and

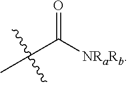

98. The compound according to claim 97, wherein R_{z} is selected from hydrogen, —CN, hydroxy, methyl, methoxy, ethoxy, propoxy, difluoromethoxy, benzyloxy, cyclopentaneoxy, and —C(O)NH₂.

99. The compound according to claim 97, wherein W is selected from a bond, 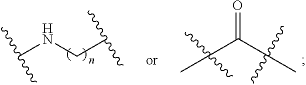
and n is selected from 0 or 1.

100. The compound according to claim 1, wherein ring A is selected from:

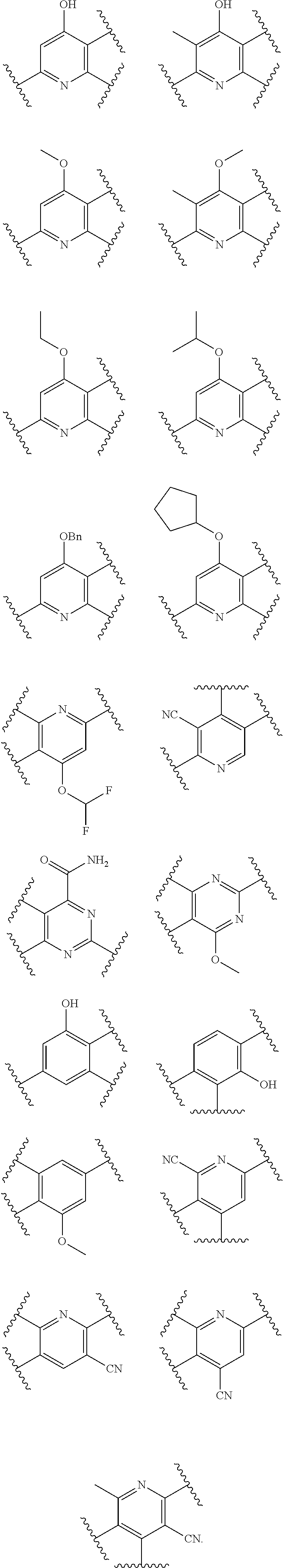

101. The compound according to claim 1, wherein the compound is selected from the compound of formula (I-8a), 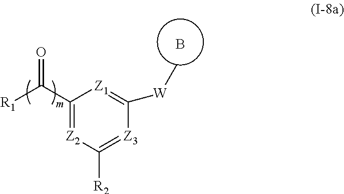
wherein Z₁, Z₂, and Z₃ are each independently selected from N or ═CR_{z}—; and
R_{z} is selected from hydrogen, halogen, —CN, hydroxy, C₁₋₆ alkyl, halogen substituted C₁₋₆ alkyl, C₁₋₆ alkoxy, halogen substituted C₁₋₆ alkoxy, C₆₋₁₀ aryl substituted C₁₋₆ alkoxy, —OC₃₋₁₀ cycloalkyl, C₃₋₁₀ cycloalkyl, —O—C₁₋₆ alkyl-(C₃₋₁₀ cycloalkyl), —O—(C₂₋₁₀ heterocycloalkyl), C₂₋₁₀ heterocycloalkyl, —O—C₁₋₆ alkyl-(C₂₋₁₀ heterocycloalkyl), C₂₋₆ alkenyl, C₂₋₆ alkynyl, —NR_{c}R_{d}, and

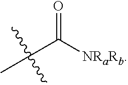

102. The compound according to claim 101, wherein R_{z} is selected from hydrogen or C₁₋₆ alkoxy.

103. The compound according to claim 101, wherein W is selected from a bond,

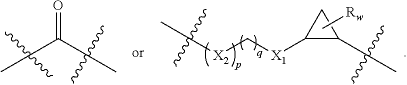

104. The compound according to claim 103, wherein X₂ is —O—; p is selected from 0 or 1; q is selected from 1, 2, 3, or 4; X₁ is —NH—; and R_{W} is H.

105. The compound according to claim 1, wherein ring A is selected from

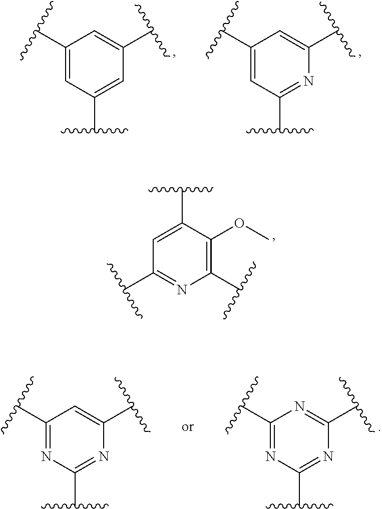 [SMILES: *c1cc(*)cc(*)c1 ; *c1cc(*)nc(*)c1 ; *c1cc(*)c(OC)c(*)n1 ; *c1cc(*)nc(*)n1 ; *c1nc(*)nc(*)n1]

106. The compound according to claim 1, wherein the compound is selected from the compound of formula (I-5a), 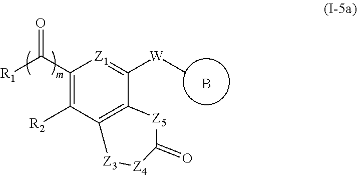
wherein Z₁ is N; Z₃, Z₄, and Z₅ are each independently selected from O, —NR₃— or —CR_{y}R_{z}—;
R₃ is selected from hydrogen or C₁₋₆ alkyl;
R_{y} and R_{z} are independently selected from hydrogen, halogen, —CN, hydroxy, C₁₋₆ alkyl, halogen substituted C₁₋₆ alkyl, C₁₋₆ alkoxy, halogen substituted C₁₋₆ alkoxy, C₆₋₁₀ aryl substituted C₁₋₆ alkoxy, —OC₃₋₁₀ cycloalkyl, C₃₋₁₀ cycloalkyl, —O—C₁₋₆ alkyl-(C₃₋₁₀ cycloalkyl), —O—(C₂₋₁₀ heterocycloalkyl), C₂₋₁₀ heterocycloalkyl, —O—C₁₋₆ alkyl-(C₂₋₁₀ heterocycloalkyl), C₂₋₆ alkenyl, C₂₋₆ alkynyl, —NR_{c}R_{d}, and 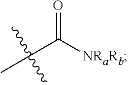
and W is a bond.

107. The compound according to claim 106, wherein R_{y} and R_{z} are both hydrogen.

108. The compound according to claim 1, wherein ring A is

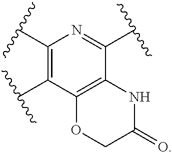 [SMILES: *c1nc(*)c2c(c1*)OCC(=O)N2]

109. The compound according to claim 1, wherein ring B is selected from C₆₋₁₀ aryl, C₃₋₁₀ cycloalkyl or C₂₋₁₀ heterocycloalkyl; the heterocycloalkyl contains 1 to 2 N heteroatoms; the cycloalkyl and the heterocycloalkyl are monocyclic, bicyclic or polycyclic group; the bicyclic and polycyclic group are bridge ring or fused ring; and
the hydrogen of the aryl, cycloalkyl and heterocycloalkyl ring is optionally substituted by 0 to 2 substituents selected from halogen, —CN, hydroxy, C₁₋₆ alkyl, C₁₋₆ alkoxy, C₂₋₁₀ heterocycloalkyl or —NR_{c}R_{d}, wherein the C₂₋₁₀ heterocycloalkyl as a substituent contains 1 to 2 heteroatoms selected from N or O; and R_{c} and R_{d} are each independently selected from H, C₁₋₆ alkyl, hydroxy substituted C₁₋₆ alkyl, and C₁₋₆ alkoxy substituted C₁₋₆ alkyl.

110. The compound according to claim 109, wherein ring B is selected from 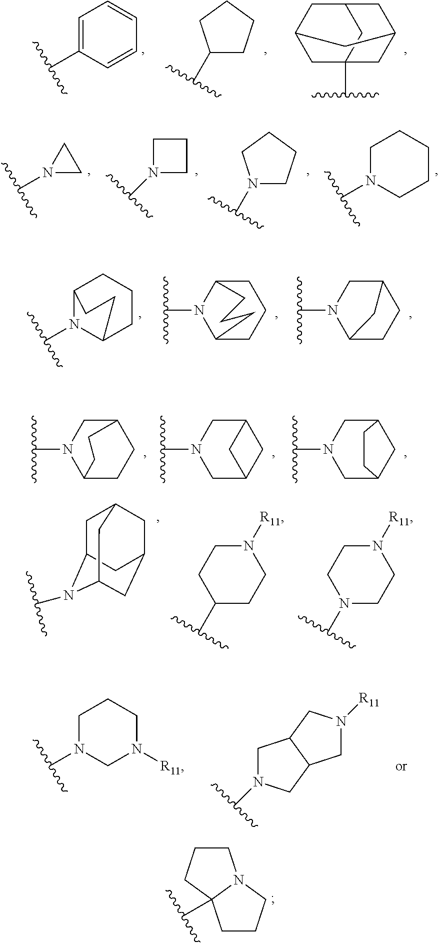
R₁₁ is selected from H, or C₁₋₆ alkyl, and the hydrogen of the ring is optionally substituted by 0 to 2 substituents selected from halogen, —CN, hydroxy, C₁₋₆ alkyl, C₁₋₆ alkoxy, 6-membered heterocycloalkyl or —NR_{c}R_{d}, wherein the 6-membered heterocycloalkyl as a substituent contains 1 to 2 heteroatoms selected from N and 0; and R_{c} and R_{d} are each independently selected from H, C₁₋₆ alkyl, hydroxy substituted C₁₋₆ alkyl, and C₁₋₃ alkoxy substituted C₁₋₃ alkyl.

111. The compound according to claim 109, wherein the hydrogen of the ring is optionally substituted by 0 to 2 substituents selected from —F, —CN, hydroxy, methyl, methoxy, —NH₂, —NHCH₃, —N(CH₃)₂,

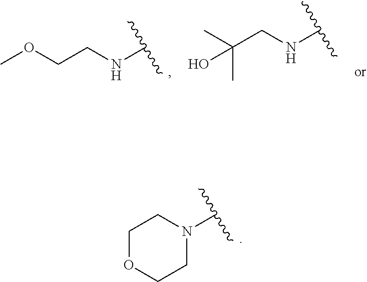

112. The compound according to claim 1, wherein ring B is selected from

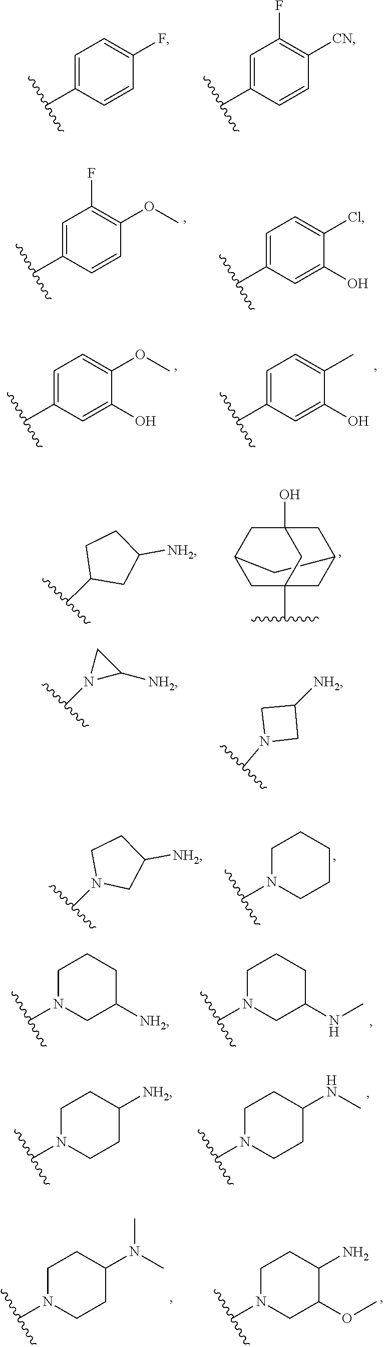

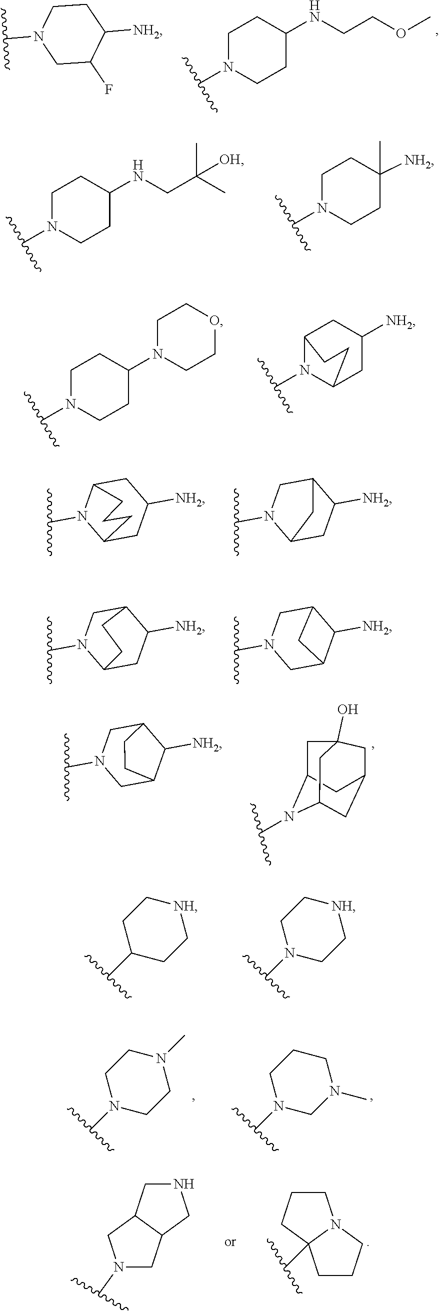

113. The compound according to claim 1, wherein ring B is selected from

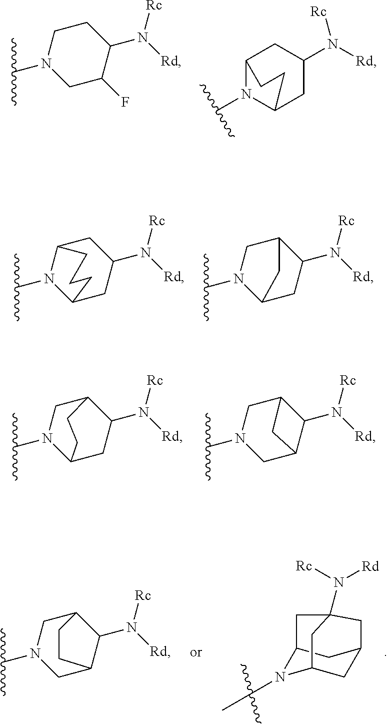

114. The compound according to claim 113, wherein R_{c} and R_{d} are each independently selected from H, C₁₋₆ alkyl, hydroxy substituted C₁₋₆ alkyl, or C₁₋₆ alkoxy substituted C₁₋₆ alkyl.

115. The compound according to claim 1, wherein R₁ and/or R₂ is selected from C₆₋₁₀ aryl, 5-10-membered heteroaryl, —OC₆₋₁₀ aryl, —O(5-10-membered heteroaryl), and C₆₋₁₀ aryl fused C₂₋₁₀ heterocycloalkyl; the C₆₋₁₀ aryl, the 5-10-membered heteroaryl, the —OC₆₋₁₀ aryl, the —O(5-10-membered heteroaryl), and the C₆₋₁₀ aryl fused C₂₋₁₀ heterocycloalkyl are optionally substituted by 0 to 4 substituents selected from —CN, —CH₂CN, halogen, —S(O)₂R₆, C₁₋₆ alkyl, halogen substituted C₁₋₆ alkyl, hydroxy, C₁₋₆ alkoxy, halogen substituted C₁₋₆ alkoxy, —NH₂, hydroxy substituted C₁₋₆ alkyl, hydroxy substituted C₂₋₆ alkynyl, hydroxy substituted C₁₋₆ alkoxy, 5-10-membered heteroaryl or C₂₋₁₀ heterocycloalkyl; the C₆₋₁₀ aryl fused C₂₋₁₀ heterocycloalkyl can be substituted by ═O; the heteroaryl and the heterocycloalkyl each contain 1 to 3 heteroatoms selected from N, O or S; and R₆ is selected from C₁₋₆ alkyl or —NH₂.

116. The compound according to claim 115, wherein R₁ and/or R₂ is selected from phenyl, 5-10-membered heteroaryl, phenoxy, and phenyl fused 5-membered heterocycloalkyl; the phenyl, the 5-10-membered heteroaryl, the phenoxy or the phenyl fused 5-membered heterocycloalkyl are optionally substituted by 0 to 4 substituents selected from —CN, —CH₂CN, halogen, —S(O)₂R₆, C₁₋₆ alkyl, halogen substituted C₁₋₆ alkyl, hydroxy, C₁₋₆ alkoxy, halogen substituted C₁₋₆ alkoxy, —NH₂, hydroxy substituted C₁₋₆ alkyl, hydroxy substituted C₂₋₆ alkynyl, hydroxy substituted C₁₋₆ alkoxy, 5-6-membered heteroaryl or 5-6-membered heterocycloalkyl; the phenyl fused 5-membered heterocycloalkyl can be substituted by ═O; the heteroaryl and the heterocycloalkyl each contain 1 to 3 heteroatoms selected from N, O or S; and R₆ is selected from C₁₋₆ alkyl or —NH₂.

117. The compound according to claim 115, wherein R₁ and/or R₂ is selected from phenyl, 5-10-membered heteroaryl, phenoxy, and phenyl fused 5-membered heterocycloalkyl; the phenyl, the 5-10-membered heteroaryl, the phenoxy or the phenyl fused 5-membered heterocycloalkyl are optionally substituted by 0 to 4 substituents selected from —CN, —CH₂CN, F, —C₁, —S(O)₂R₆, methyl, pentyl, trifluoromethyl, hydroxy, methoxy, trifluoromethoxy, —NH₂, 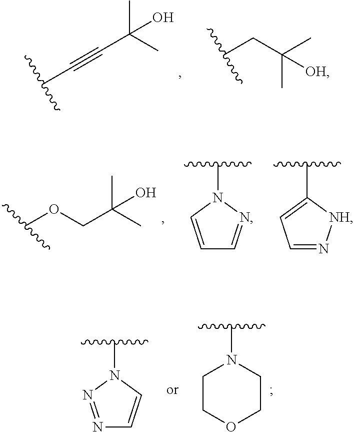
the phenyl fused 5-membered heterocycloalkyl can be substituted by ═O; the heteroaryl and the heterocycloalkyl each contain 1 to 3 heteroatoms selected from N, O or S; and R₆ is selected from methyl or —NH₂.

118. The compound according to claim 1, wherein R₁ and/or R₂ is selected from:

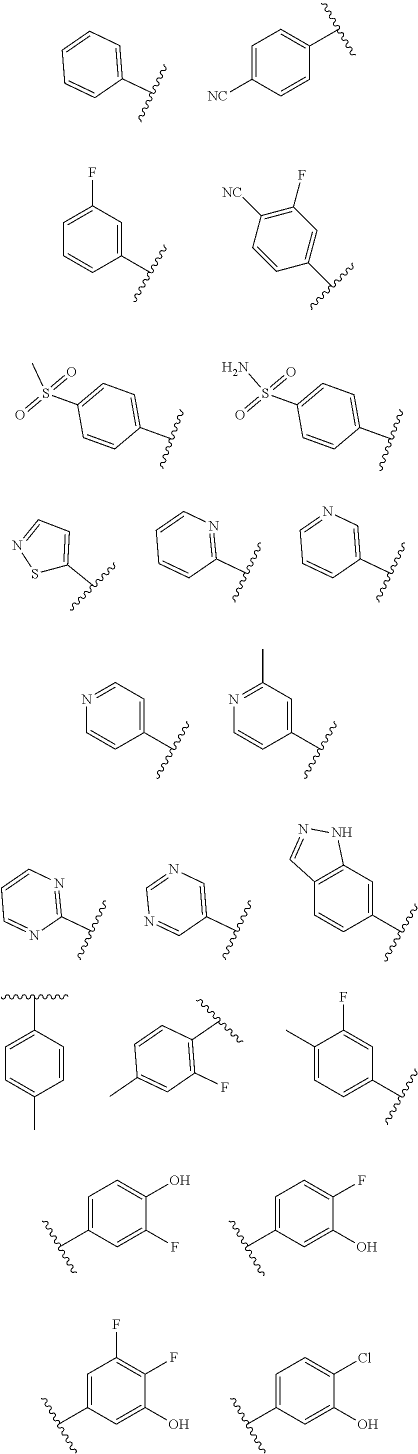

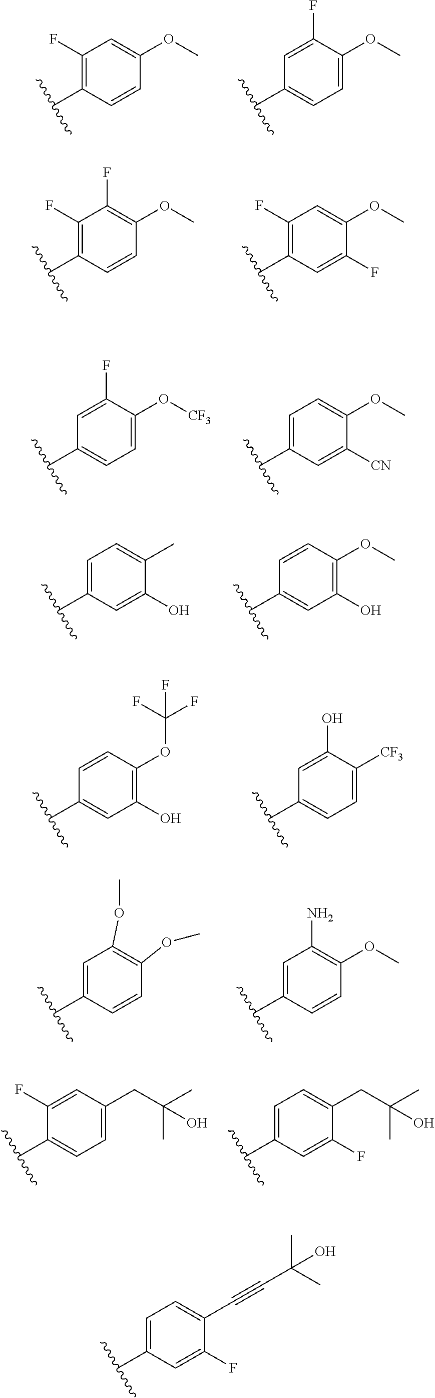

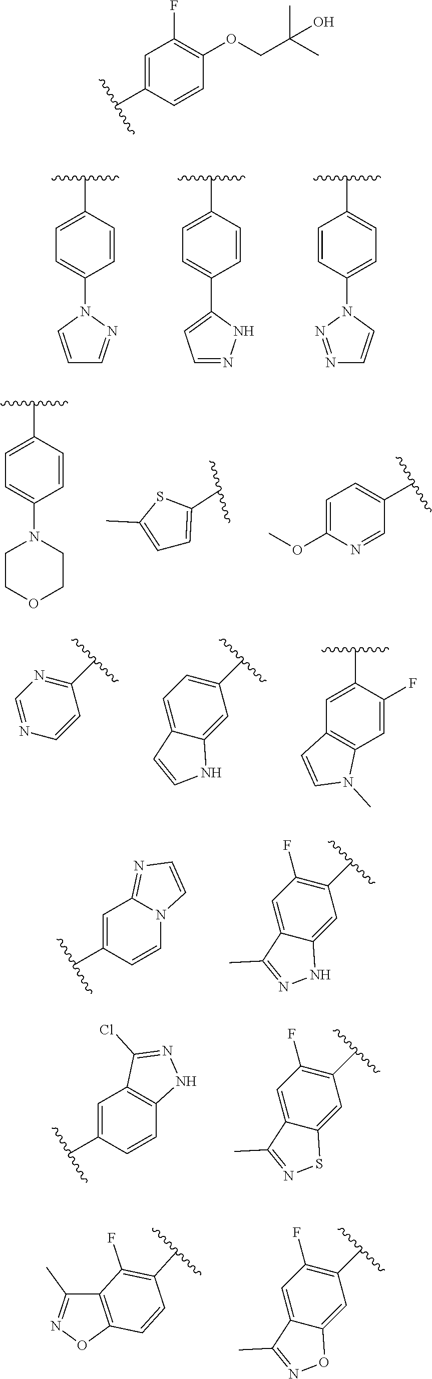

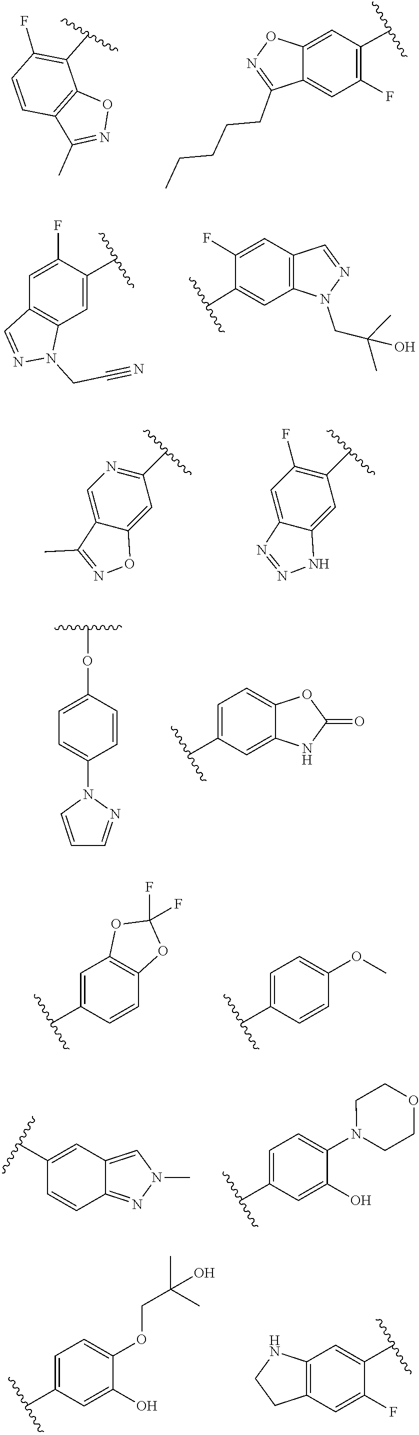

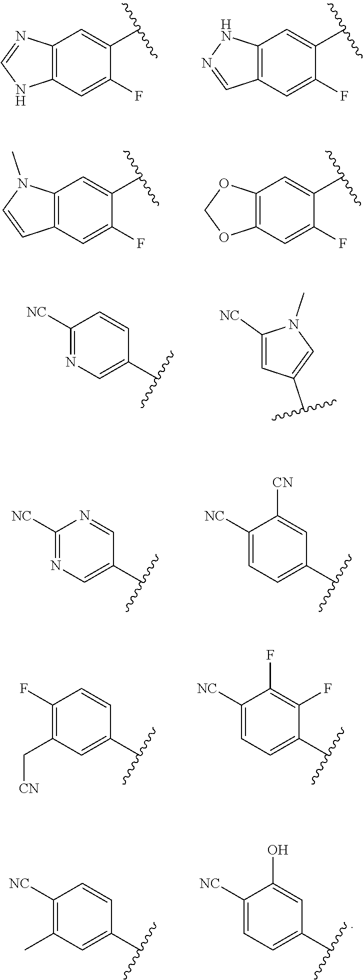

119. The compound according to claim 1, wherein one of R₁ and R₂ is —NR₄R₅; R₄ and R₅ are each independently selected from H, C₆₋₁₀ alkyl, —NH₂ substituted C₁₋₆ alkyl, C₃₋₁₀ cycloalkyl, C₂₋₁₀ heterocycloalkyl, C₆₋₁₀ aryl or 5-10-membered heteroaryl; the C₆₋₁₀ aryl and the 5-10-membered heteroaryl are optionally substituted by 0 to 4 substituents selected from halogen or C₁₋₆ alkoxy; and the C₂₋₁₀ heterocycloalkyl and the 5-10-membered heteroaryl contain 1 to 3 heteroatoms selected from N, O or S.

120. The compound according to claim 119, wherein R₄ and R₅ are each independently selected from H, C₁₋₆ alkyl, —NH₂ substituted C₁₋₆ alkyl, 6-membered heterocycloalkyl or phenyl;
the phenyl is optionally substituted by 0 to 2 substituents selected from halogen or C₁₋₆ alkoxy;
and the 6-membered heterocycloalkyl contains 1 to 2 heteroatoms selected from N or O.

121. The compound according to claim 1, wherein one of R₁ and R₂ is selected from

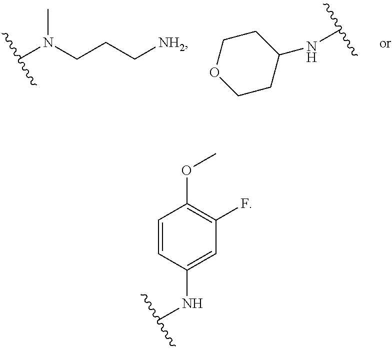

122. Compounds of the following formula, or a tautomer, mesomer, racemate, enantiomer, diastereoisomer, or mixture thereof, or a deuterated isotopic derivative, pharmaceutically acceptable hydrate, solvate, salt or cocrystal thereof,

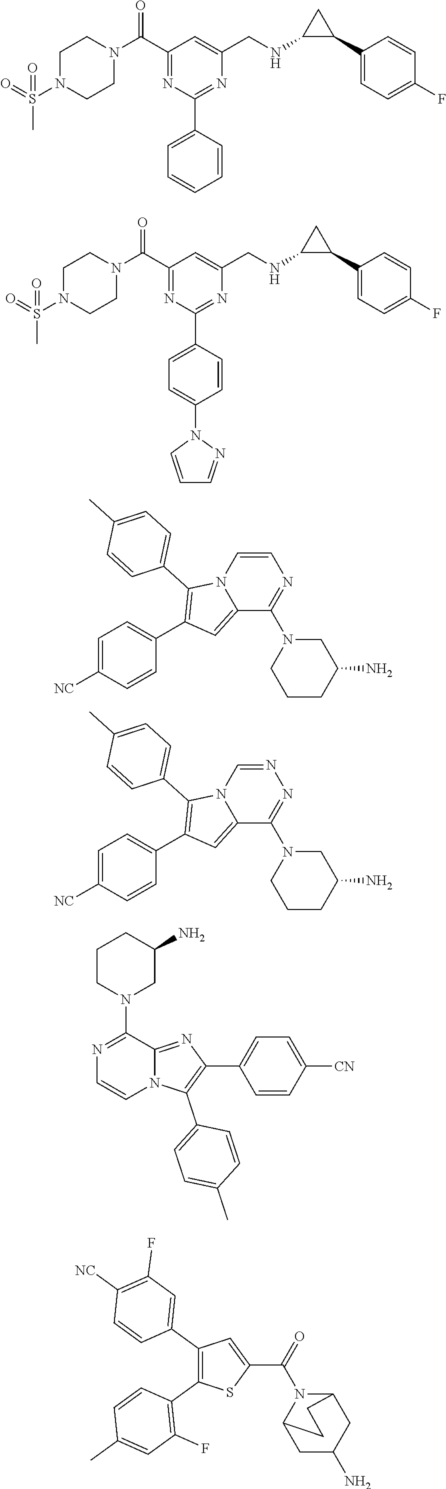

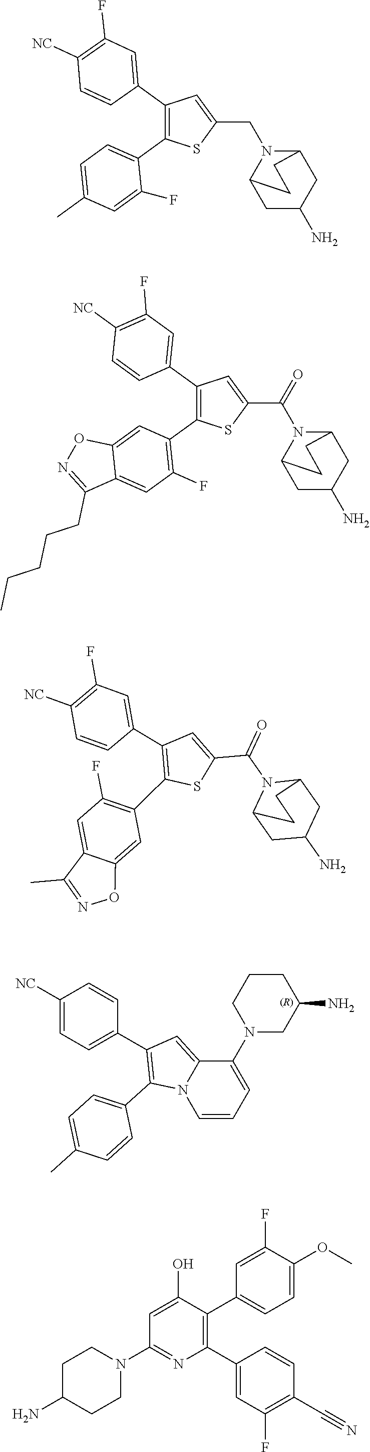

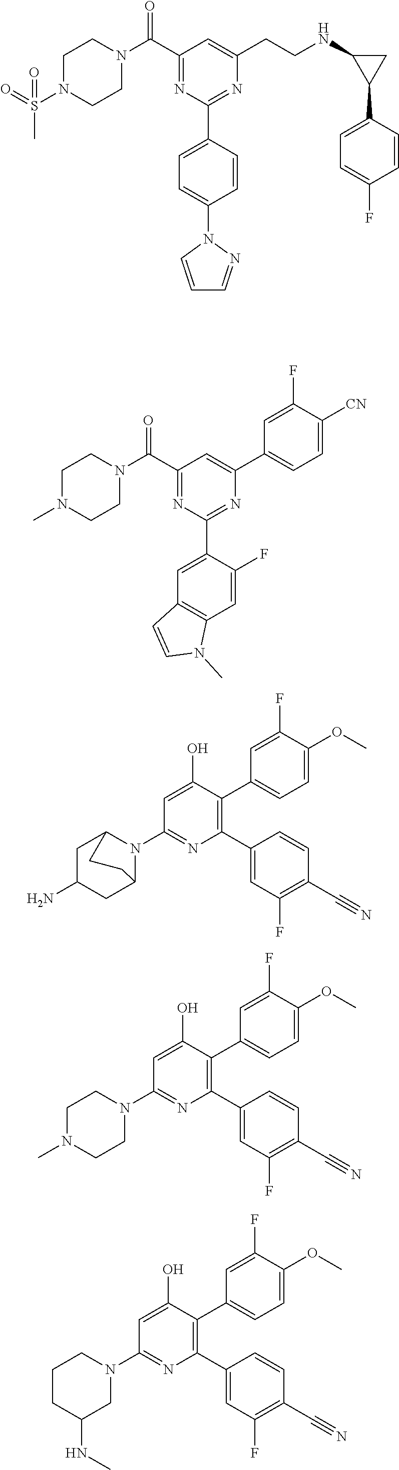

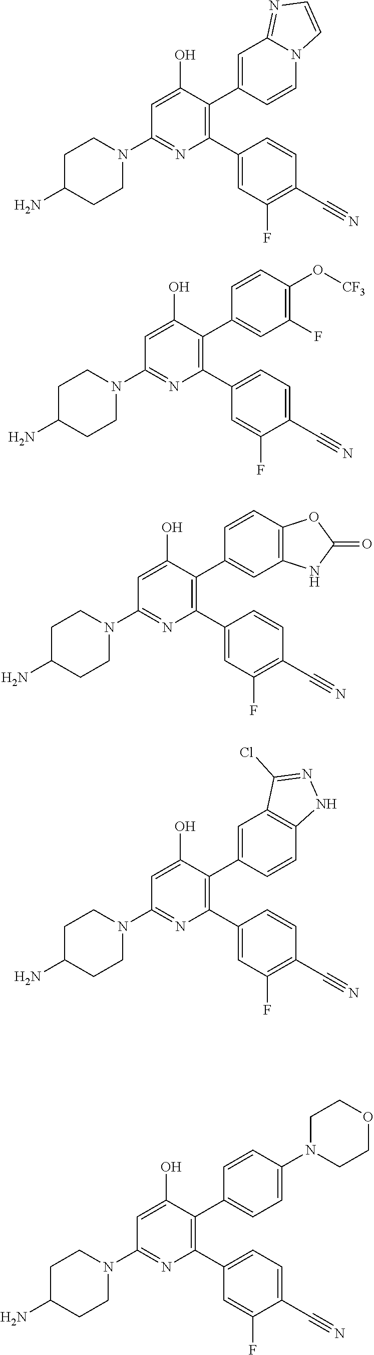

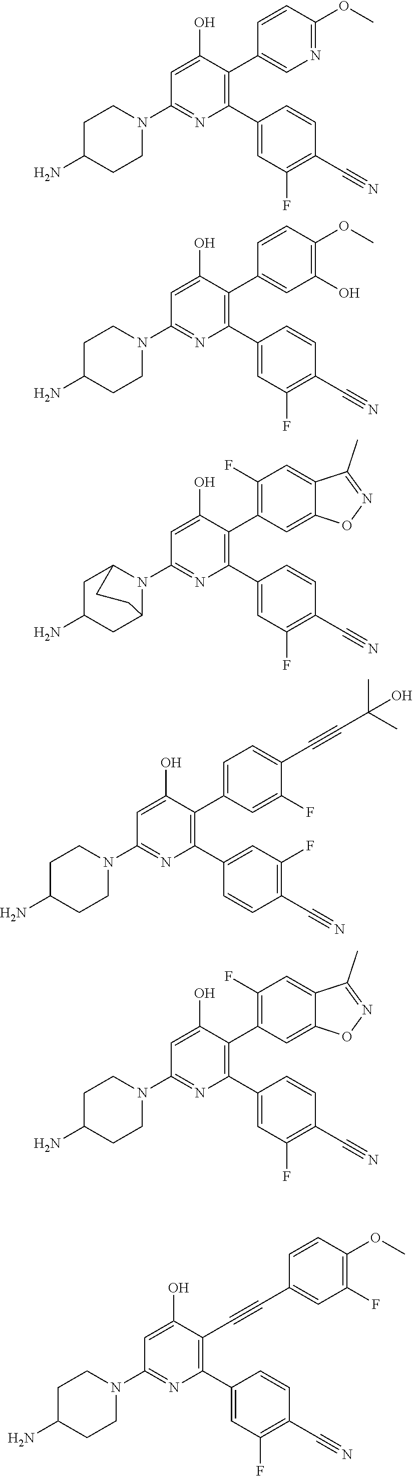

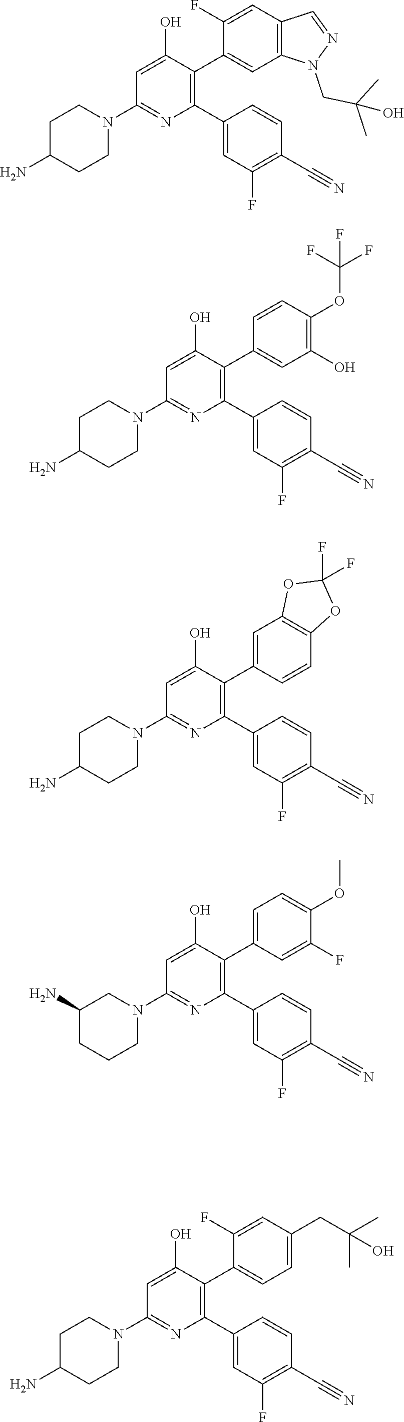

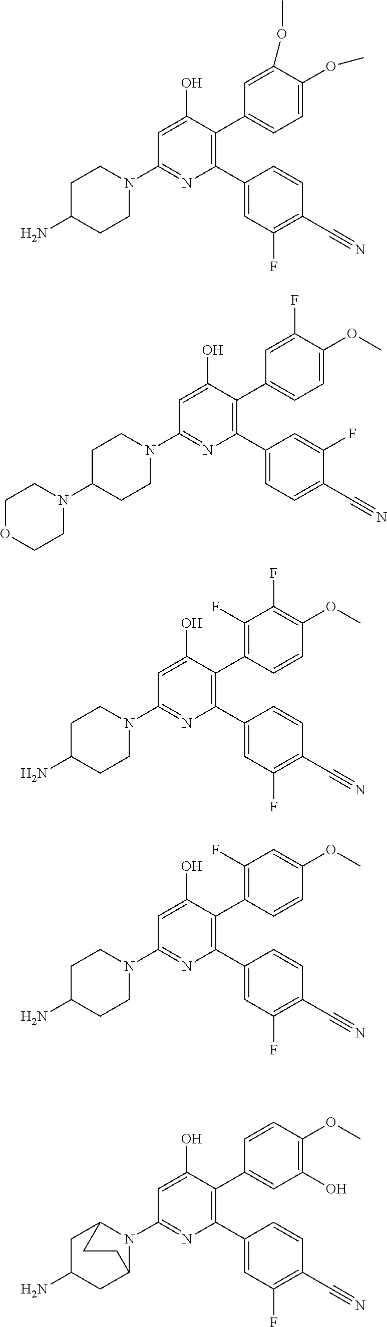

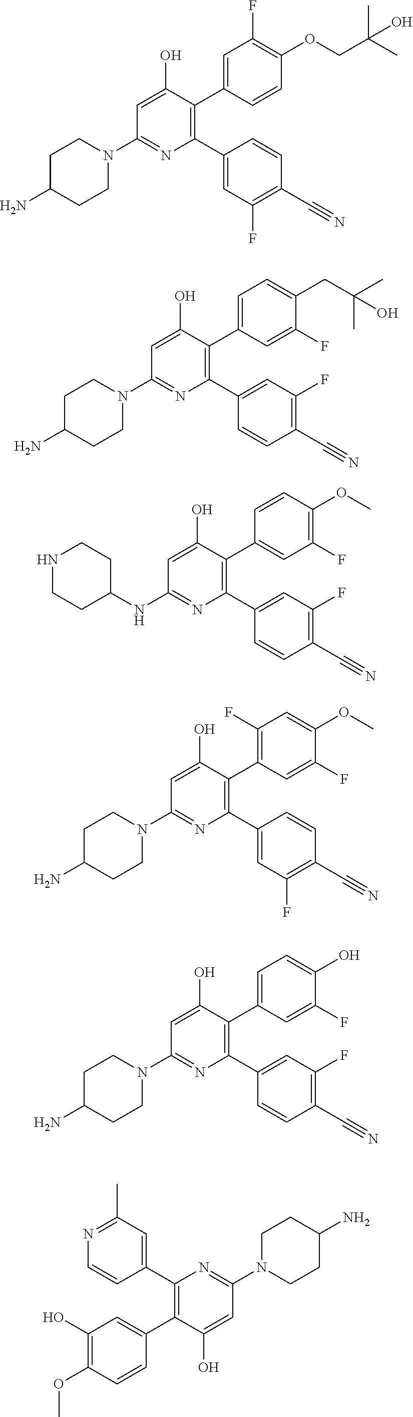

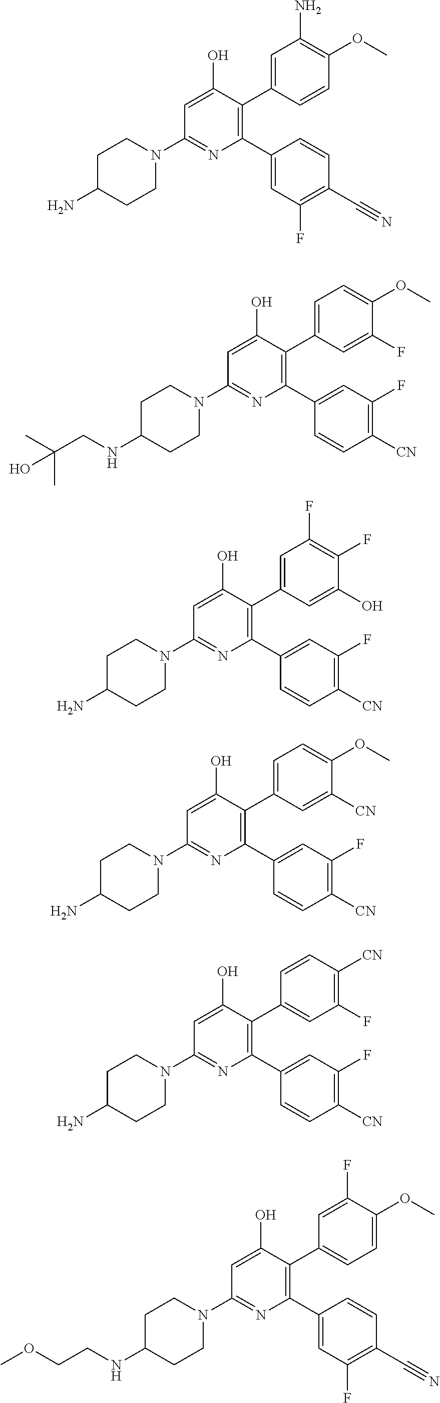

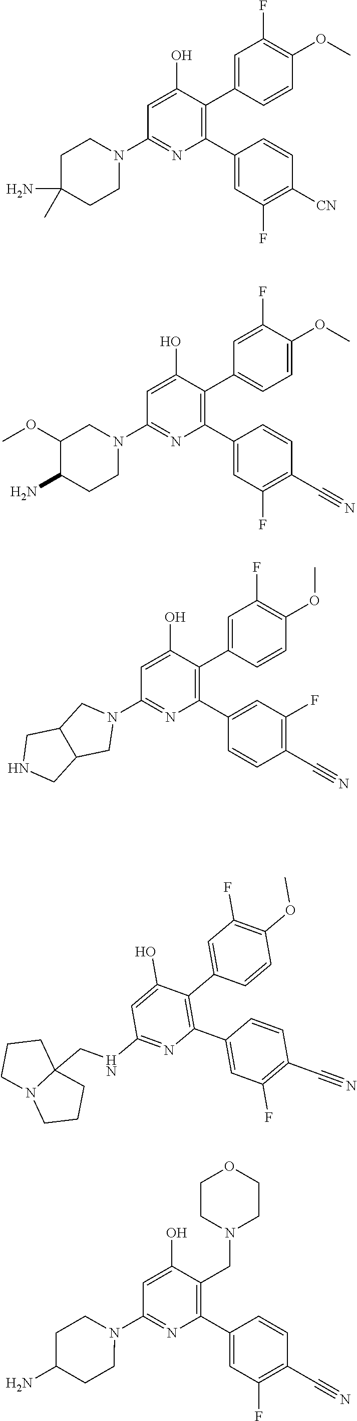

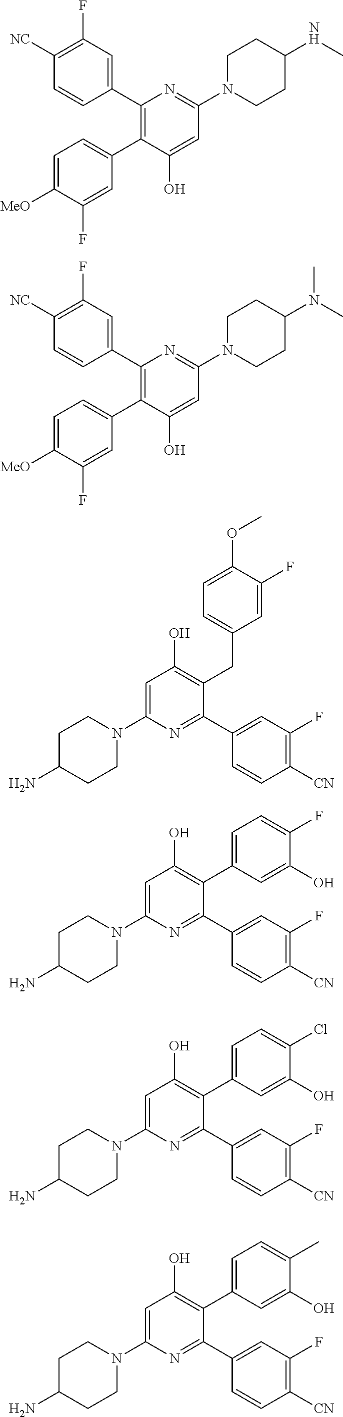

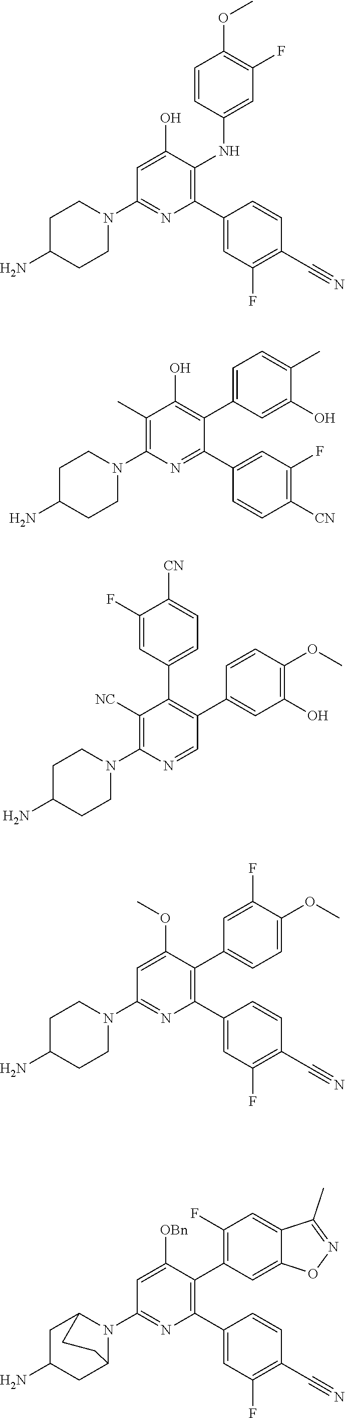 [SMILES: COc1ccc(Nc2c(O)cc(N3CCC(N)CC3)nc2-c2ccc(C#N)c(F)c2)cc1F ; Cc1ccc(-c2c(-c3ccc(C#N)c(F)c3)nc(N3CCC(N)CC3)c(C)c2O)cc1O ; COc1ccc(-c2cnc(N3CCC(N)CC3)c(C#N)c2-c2ccc(C#N)c(F)c2)cc1O ; COc1ccc(-c2c(OC)cc(N3CCC(N)CC3)nc2-c2ccc(C#N)c(F)c2)cc1F ; Cc1noc2cc(-c3c(OCc4ccccc4)cc(N4C5CCC4CC(N)C5)nc3-c3ccc(C#N)c(F)c3)c(F)cc12]

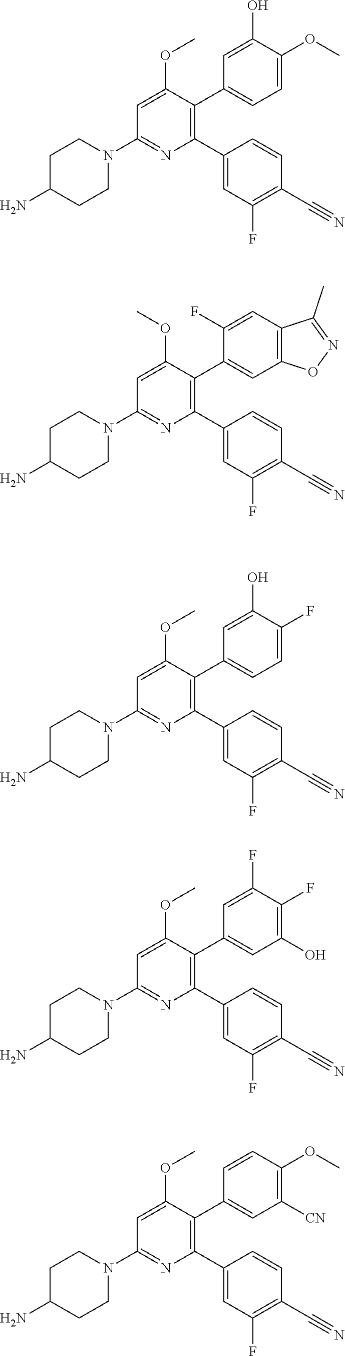

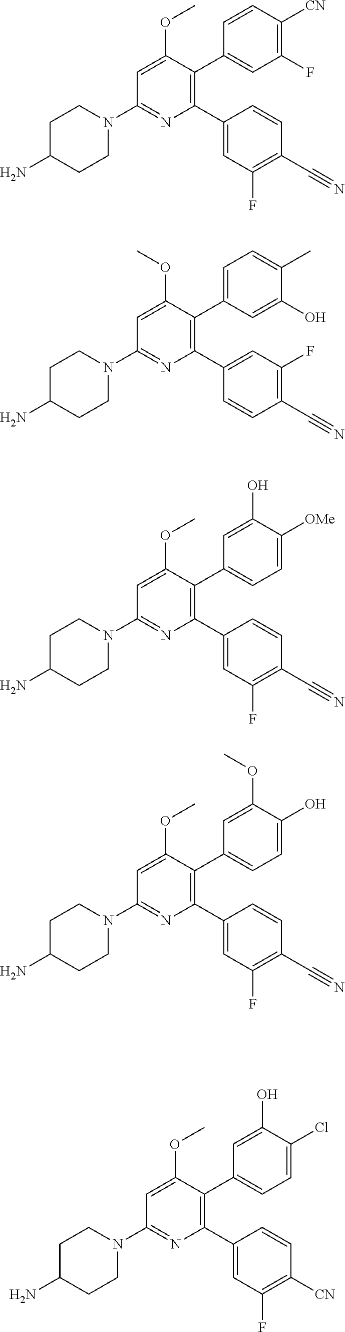

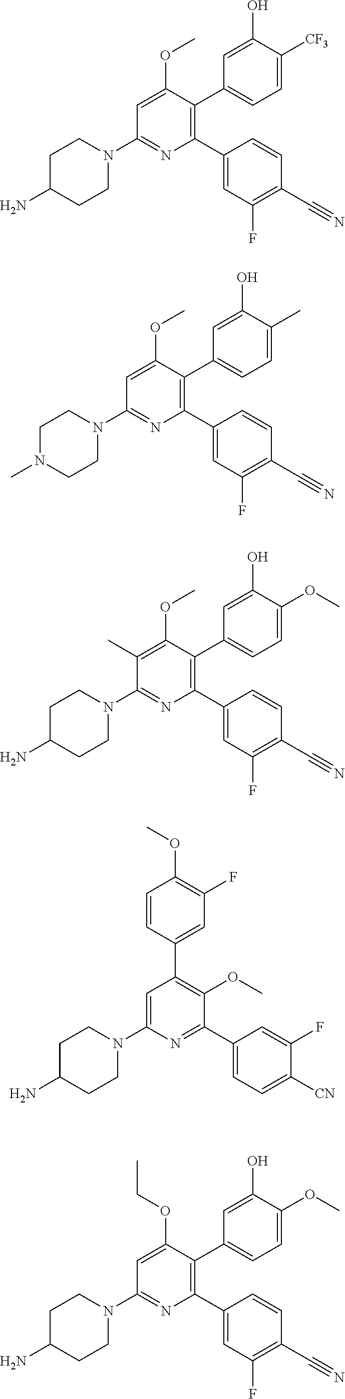

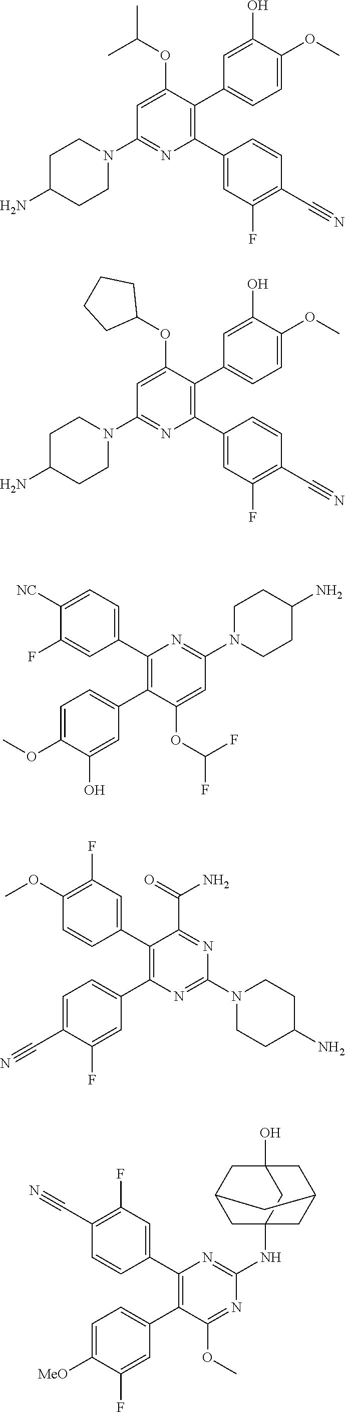

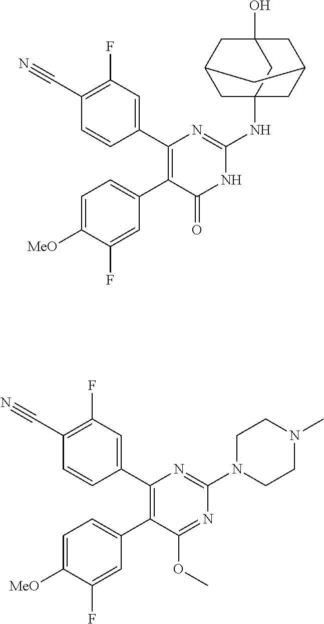 [SMILES: COc1ccc(-c2c(-c3ccc(C#N)c(F)c3)nc(NC34CC5CC(CC(O)(C5)C3)C4)[nH]c2=O)cc1F ; COc1ccc(-c2c(OC)nc(N3CCN(C)CC3)nc2-c2ccc(C#N)c(F)c2)cc1F]

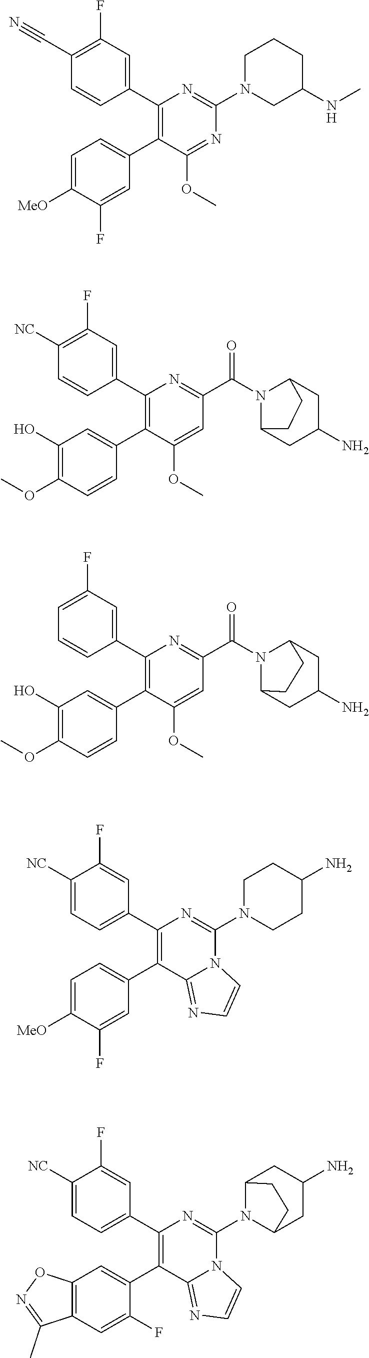

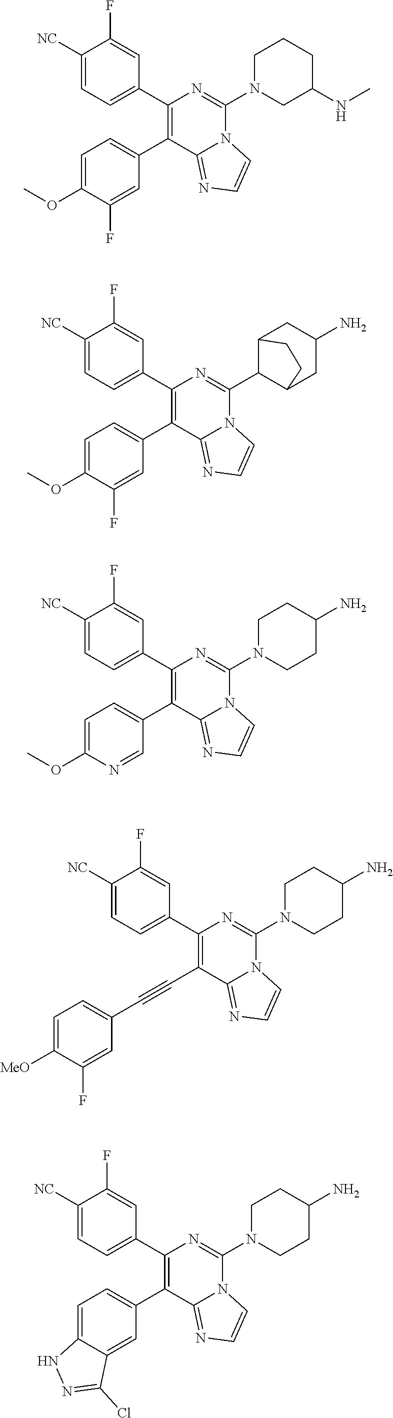

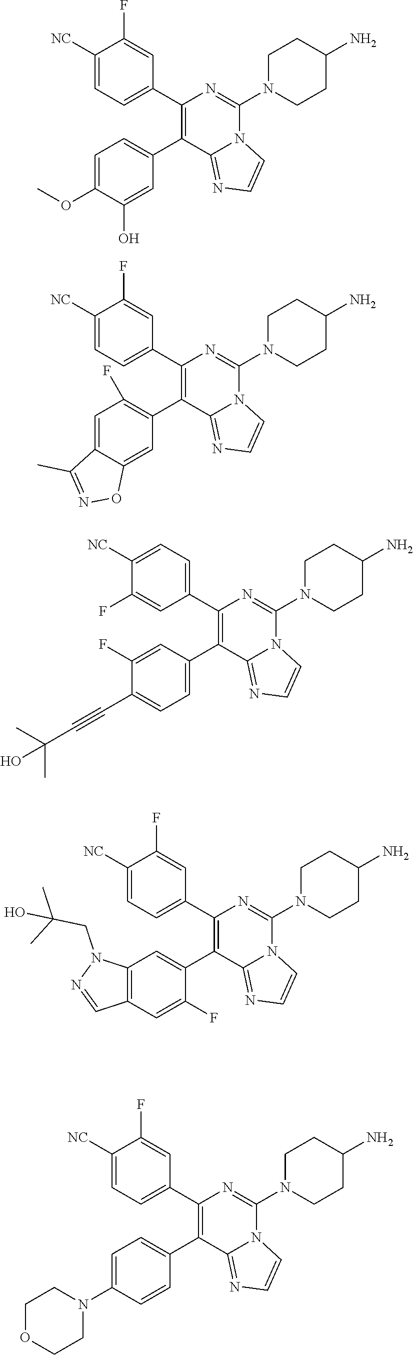

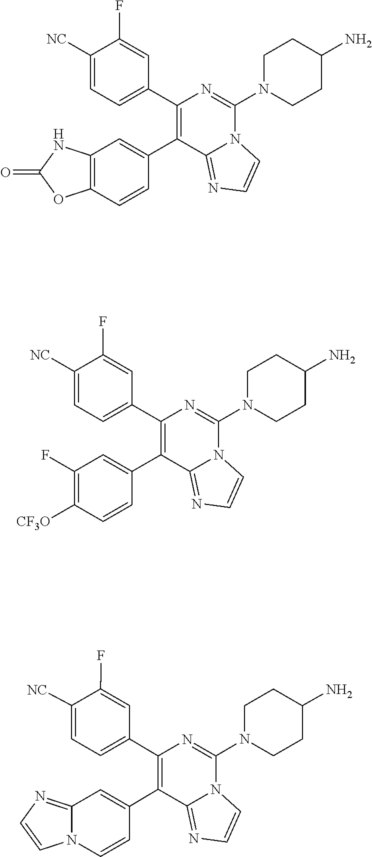

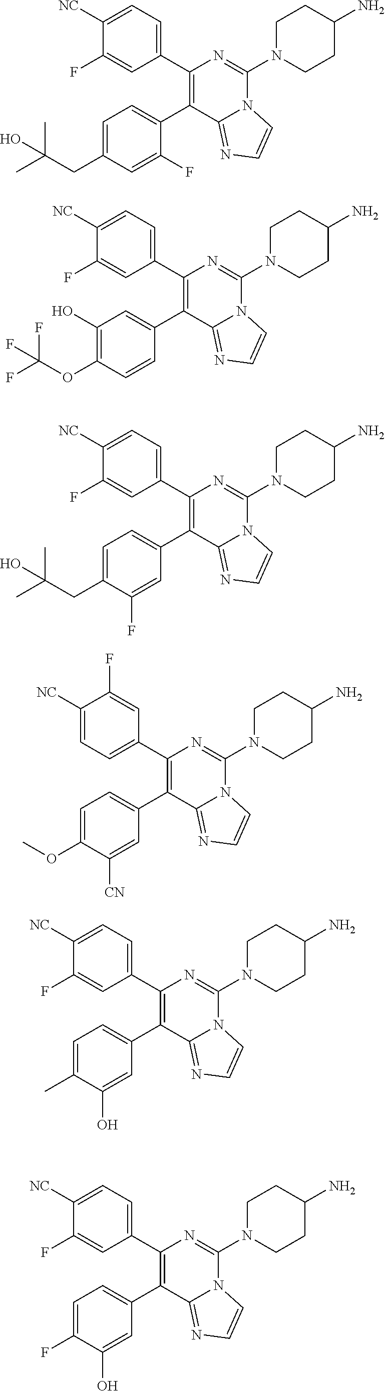

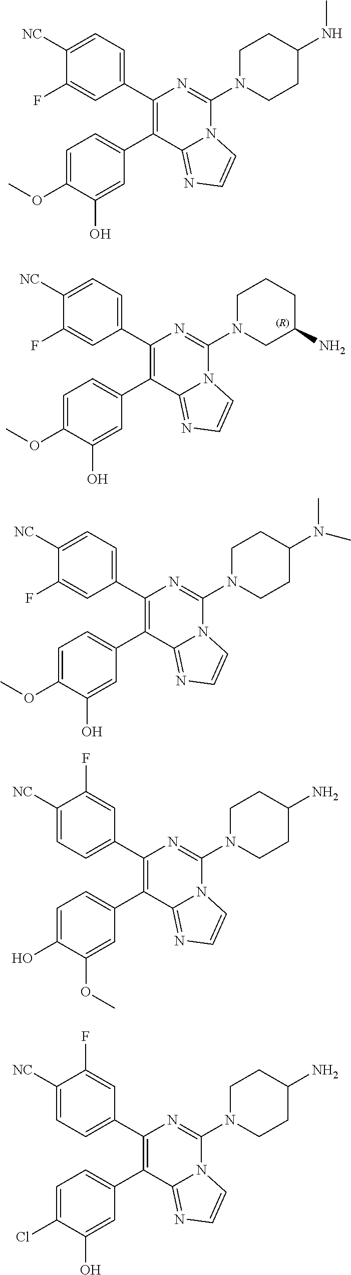

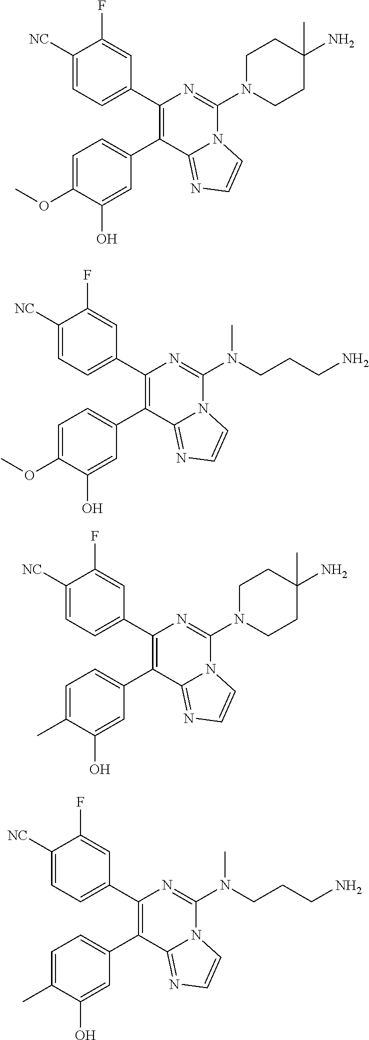

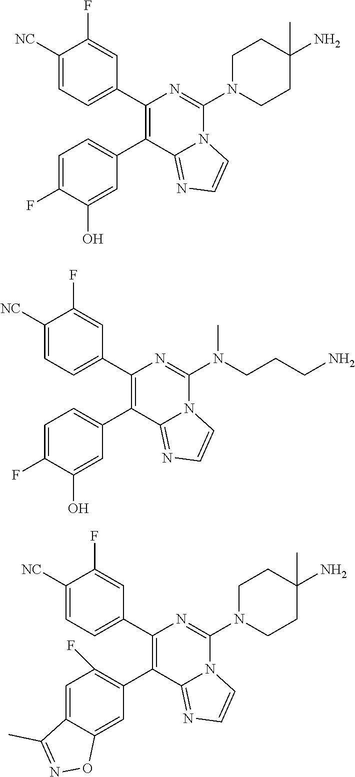

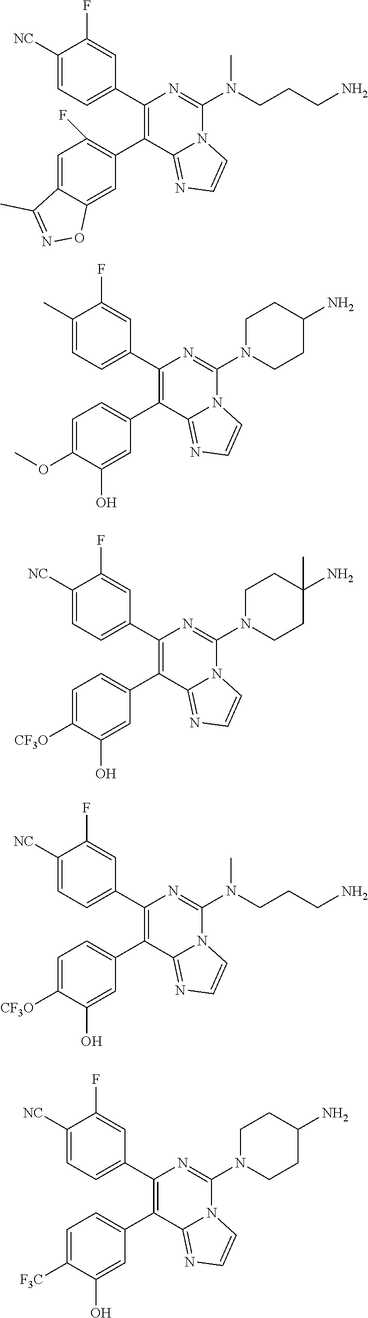

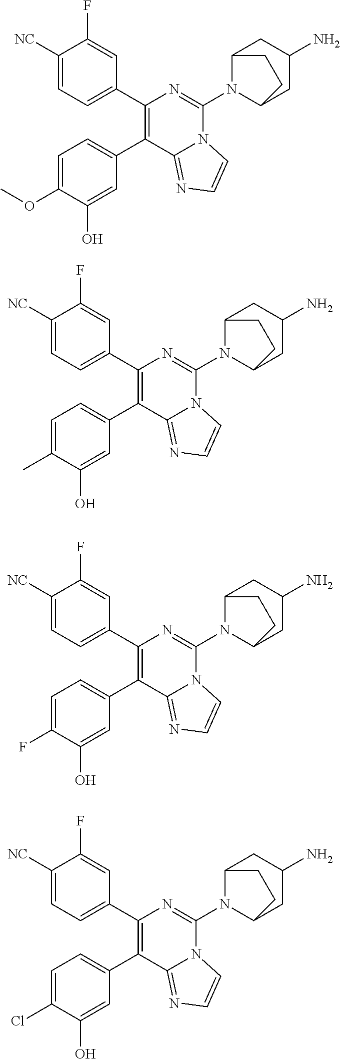

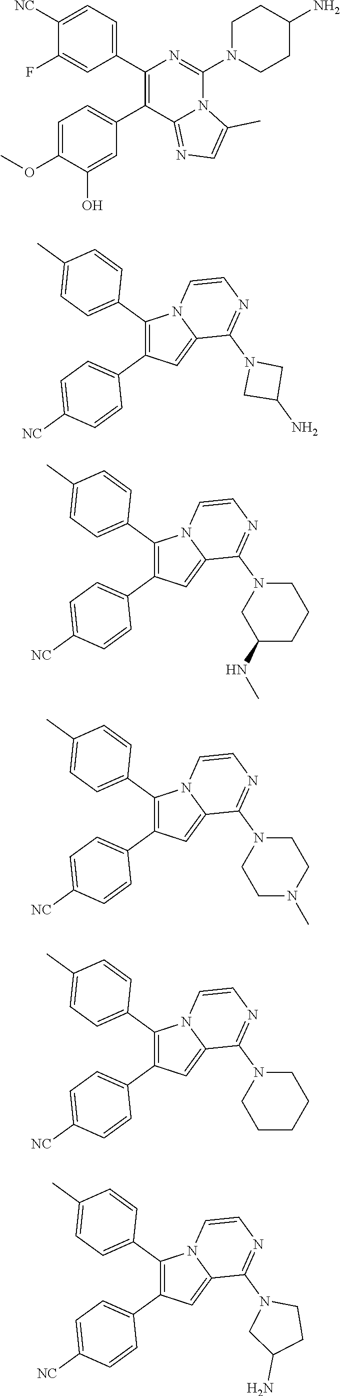

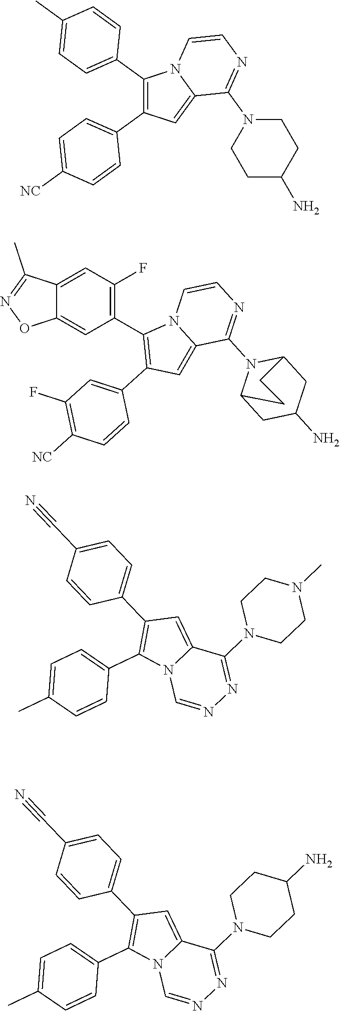

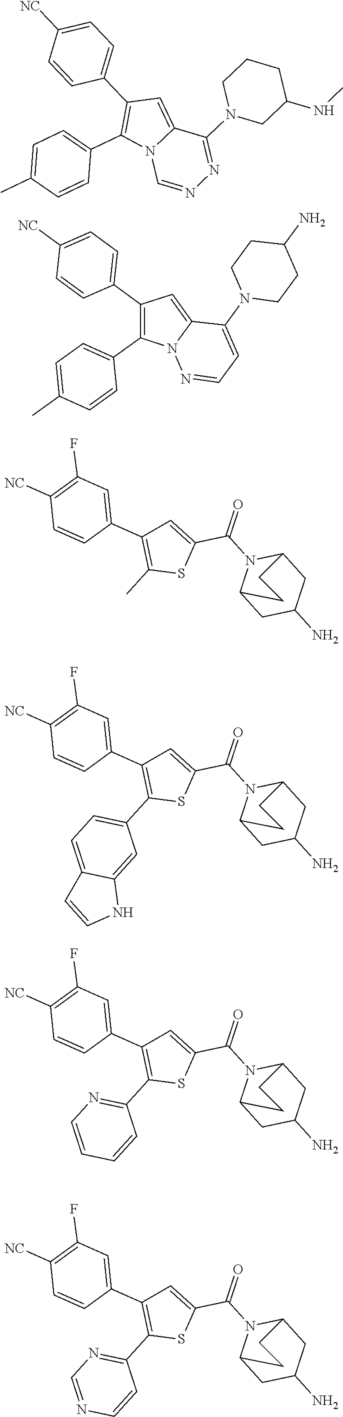

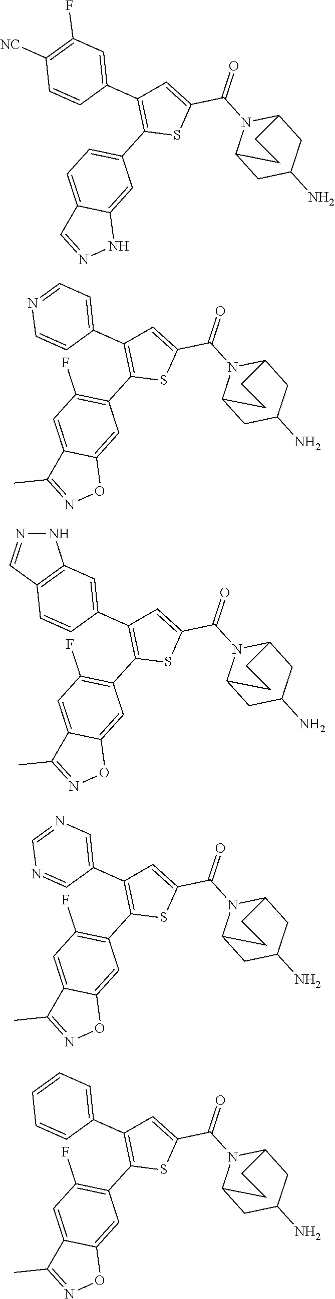

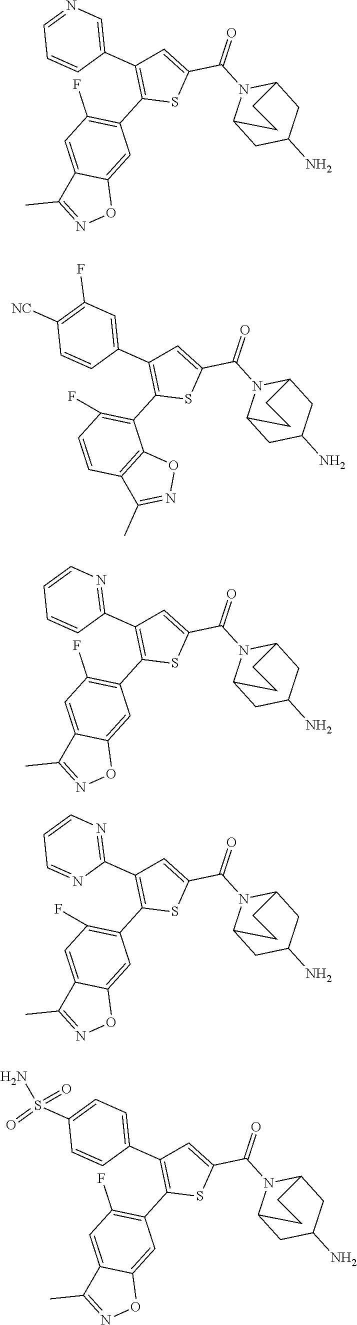

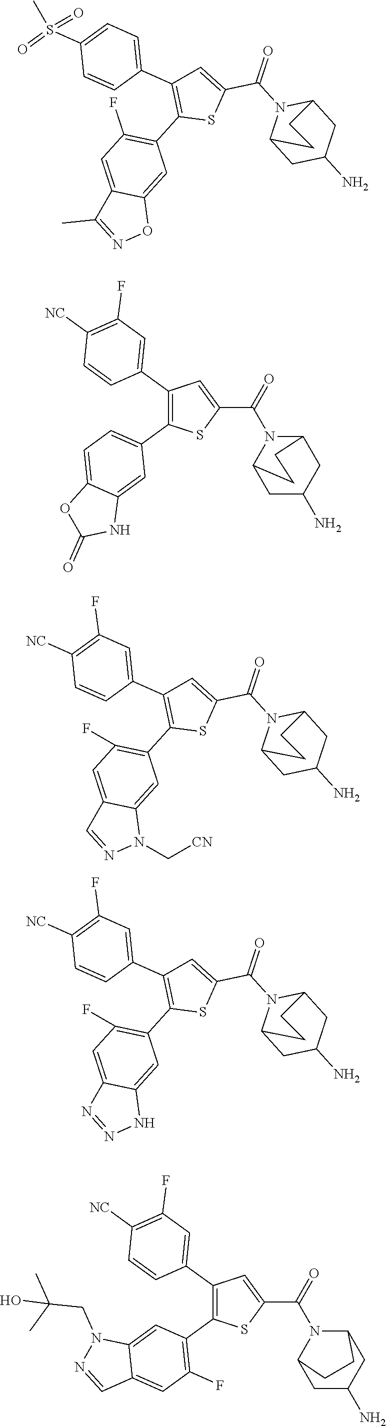

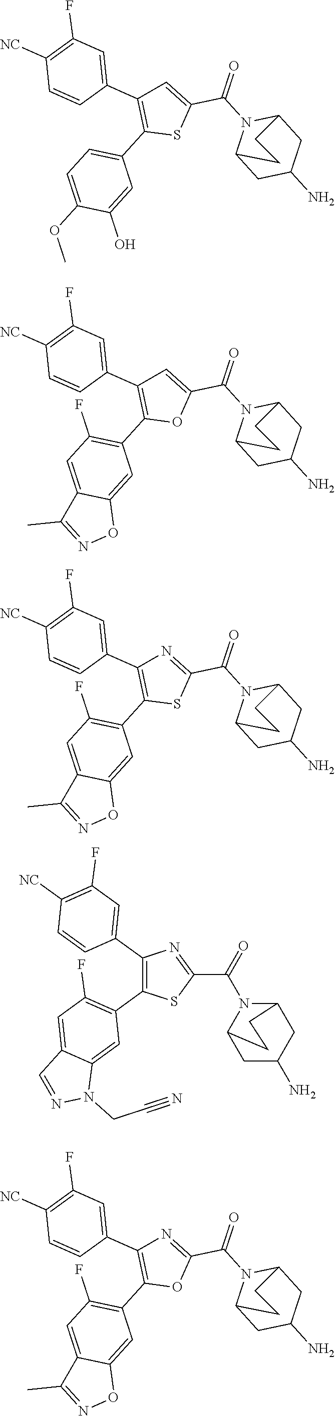

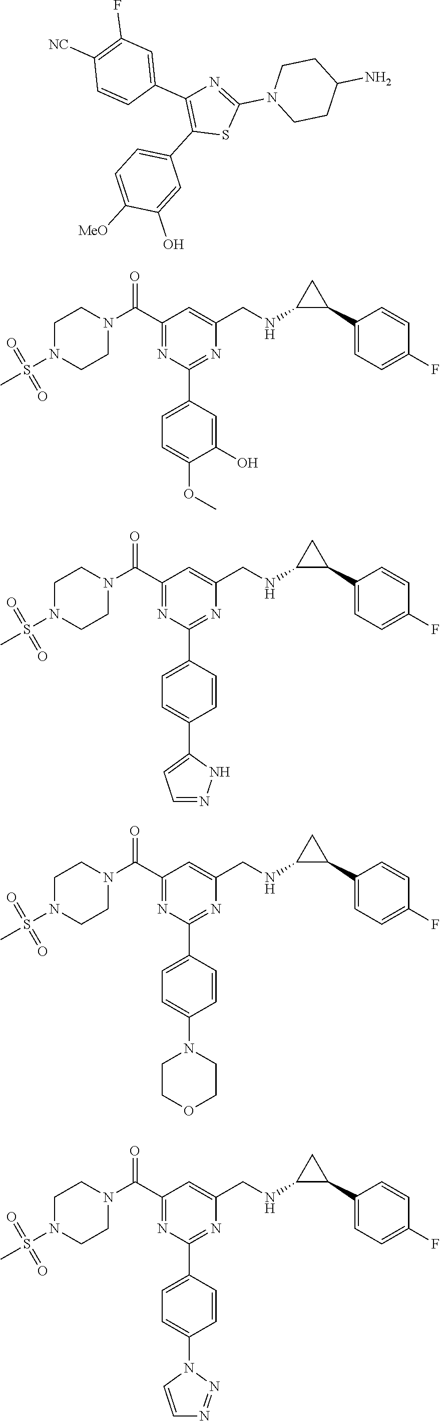 [SMILES: COc1ccc(-c2sc(N3CCC(N)CC3)nc2-c2ccc(C#N)c(F)c2)cc1O ; COc1ccc(-c2nc(CN[C@@H]3C[C@H]3c3ccc(F)cc3)cc(C(=O)N3CCN(S(C)(=O)=O)CC3)n2)cc1O ; CS(=O)(=O)N1CCN(C(=O)c2cc(CN[C@@H]3C[C@H]3c3ccc(F)cc3)nc(-c3ccc(-c4ccn[nH]4)cc3)n2)CC1 ; CS(=O)(=O)N1CCN(C(=O)c2cc(CN[C@@H]3C[C@H]3c3ccc(F)cc3)nc(-c3ccc(N4CCOCC4)cc3)n2)CC1 ; CS(=O)(=O)N1CCN(C(=O)c2cc(CN[C@@H]3C[C@H]3c3ccc(F)cc3)nc(-c3ccc(-n4ccnn4)cc3)n2)CC1]

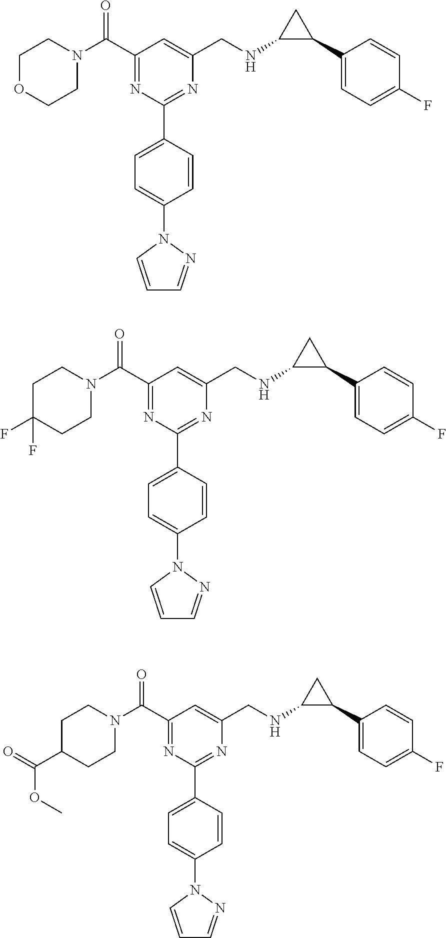 [SMILES: O=C(c1cc(CN[C@@H]2C[C@H]2c2ccc(F)cc2)nc(-c2ccc(-n3cccn3)cc2)n1)N1CCOCC1 ; O=C(c1cc(CN[C@@H]2C[C@H]2c2ccc(F)cc2)nc(-c2ccc(-n3cccn3)cc2)n1)N1CCC(F)(F)CC1 ; COC(=O)C1CCN(C(=O)c2cc(CN[C@@H]3C[C@H]3c3ccc(F)cc3)nc(-c3ccc(-n4cccn4)cc3)n2)CC1]

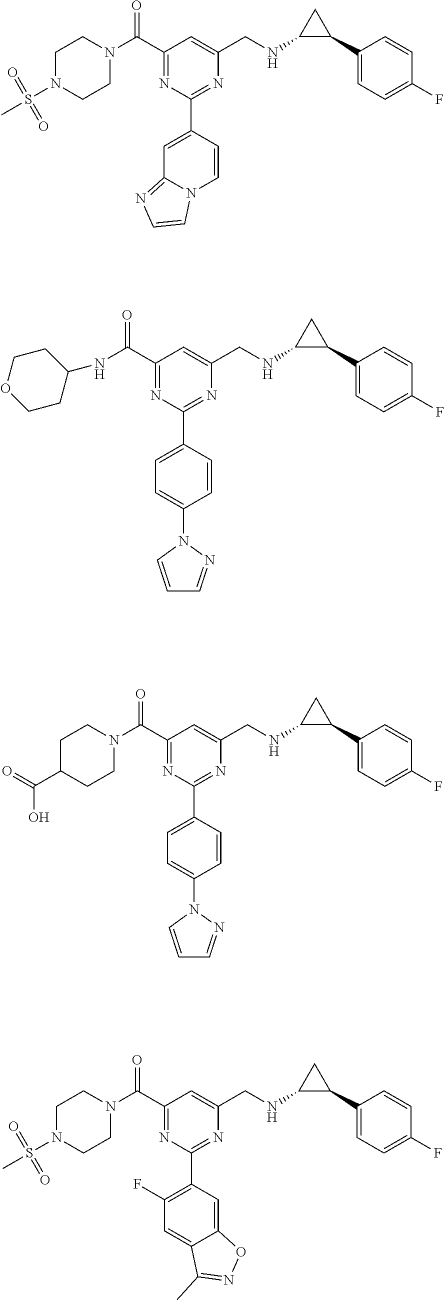 [SMILES: CS(=O)(=O)N1CCN(C(=O)c2cc(CN[C@@H]3C[C@H]3c3ccc(F)cc3)nc(-c3ccn4ccnc4c3)n2)CC1 ; O=C(NC1CCOCC1)c1cc(CN[C@@H]2C[C@H]2c2ccc(F)cc2)nc(-c2ccc(-n3cccn3)cc2)n1 ; O=C(O)C1CCN(C(=O)c2cc(CN[C@@H]3C[C@H]3c3ccc(F)cc3)nc(-c3ccc(-n4cccn4)cc3)n2)CC1 ; Cc1noc2cc(-c3nc(CN[C@@H]4C[C@H]4c4ccc(F)cc4)cc(C(=O)N4CCN(S(C)(=O)=O)CC4)n3)c(F)cc12]

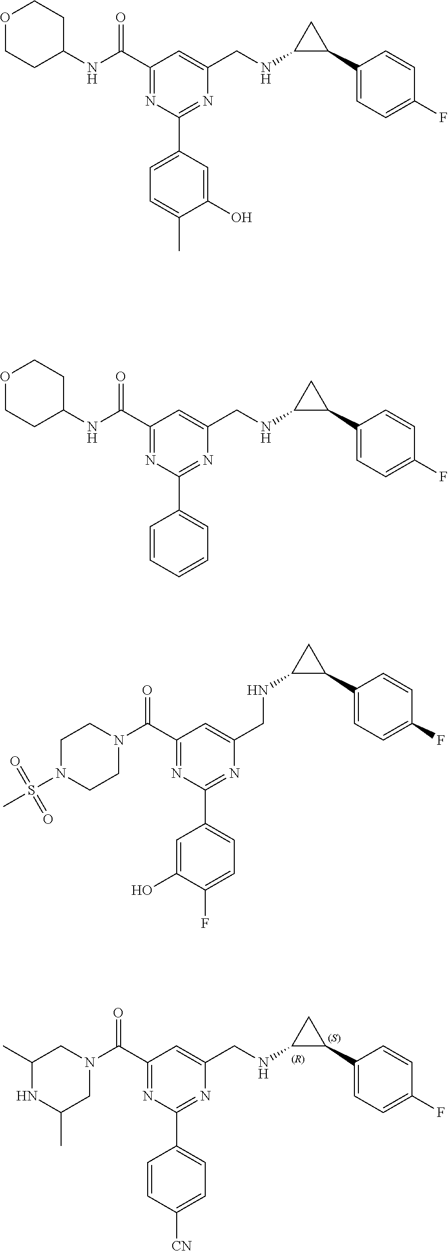

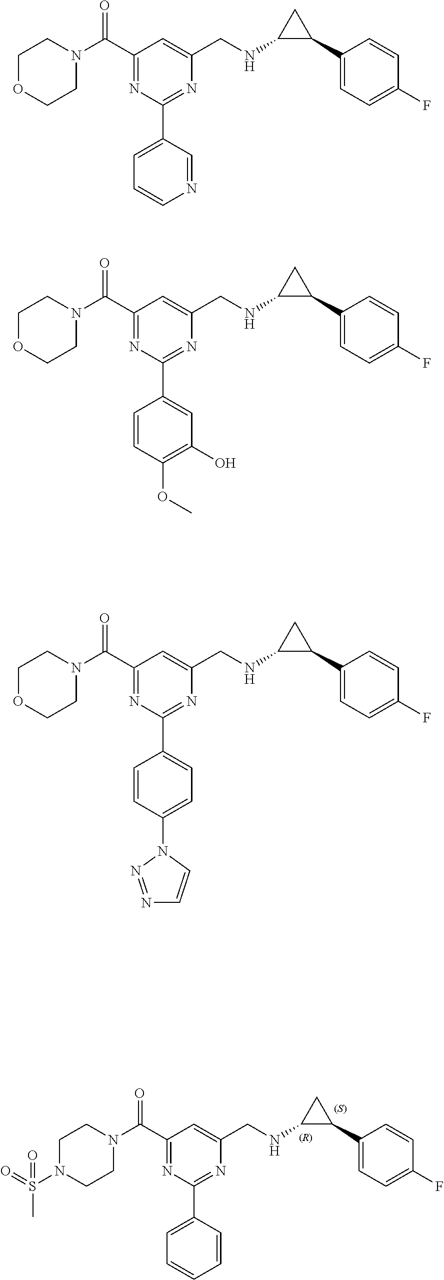

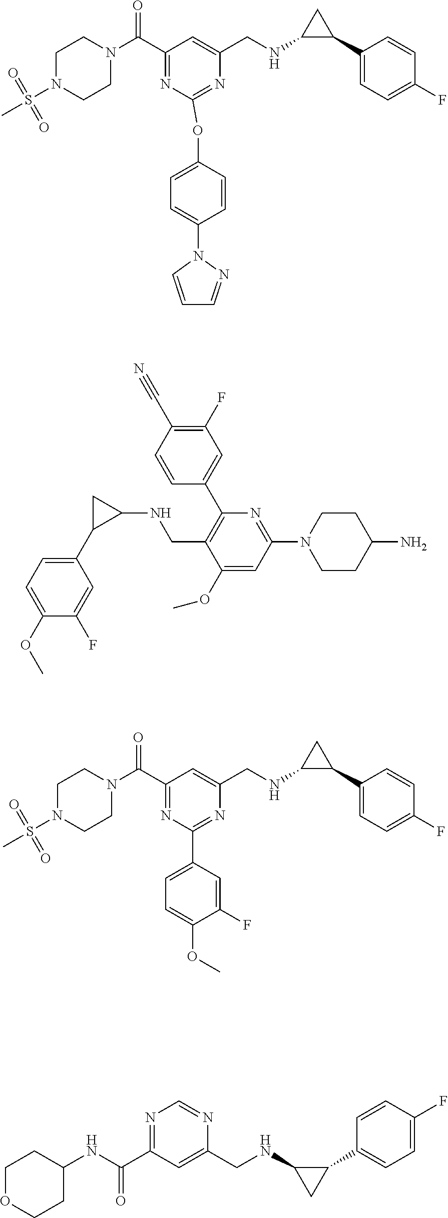 [SMILES: CS(=O)(=O)N1CCN(C(=O)c2cc(CN[C@@H]3C[C@H]3c3ccc(F)cc3)nc(Oc3ccc(-n4cccn4)cc3)n2)CC1 ; COc1ccc(C2CC2NCc2c(OC)cc(N3CCC(N)CC3)nc2-c2ccc(C#N)c(F)c2)cc1F ; COc1ccc(-c2nc(CN[C@@H]3C[C@H]3c3ccc(F)cc3)cc(C(=O)N3CCN(S(C)(=O)=O)CC3)n2)cc1F ; O=C(NC1CCOCC1)c1cc(CN[C@@H]2C[C@H]2c2ccc(F)cc2)ncn1]

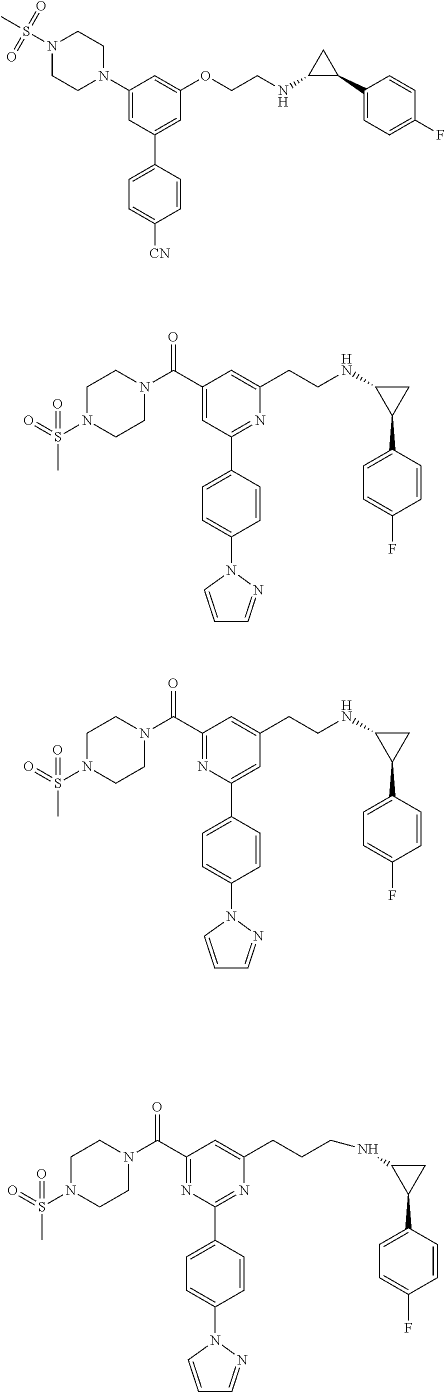

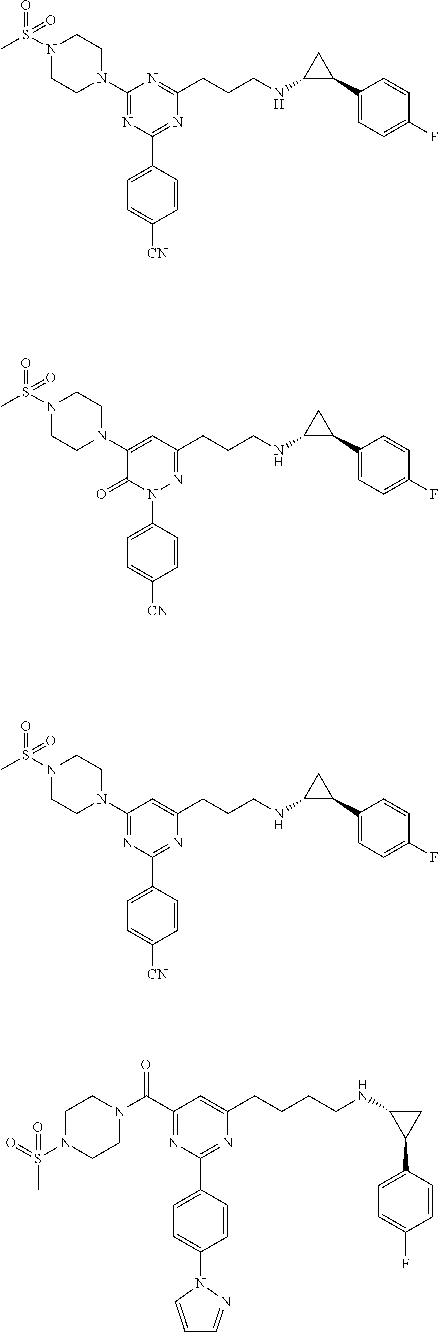 [SMILES: CS(=O)(=O)N1CCN(c2nc(CCCN[C@@H]3C[C@H]3c3ccc(F)cc3)nc(-c3ccc(C#N)cc3)n2)CC1 ; CS(=O)(=O)N1CCN(c2cc(CCCN[C@@H]3C[C@H]3c3ccc(F)cc3)nn(-c3ccc(C#N)cc3)c2=O)CC1 ; CS(=O)(=O)N1CCN(c2cc(CCCN[C@@H]3C[C@H]3c3ccc(F)cc3)nc(-c3ccc(C#N)cc3)n2)CC1 ; CS(=O)(=O)N1CCN(C(=O)c2cc(CCCCN[C@@H]3C[C@H]3c3ccc(F)cc3)nc(-c3ccc(-n4cccn4)cc3)n2)CC1]

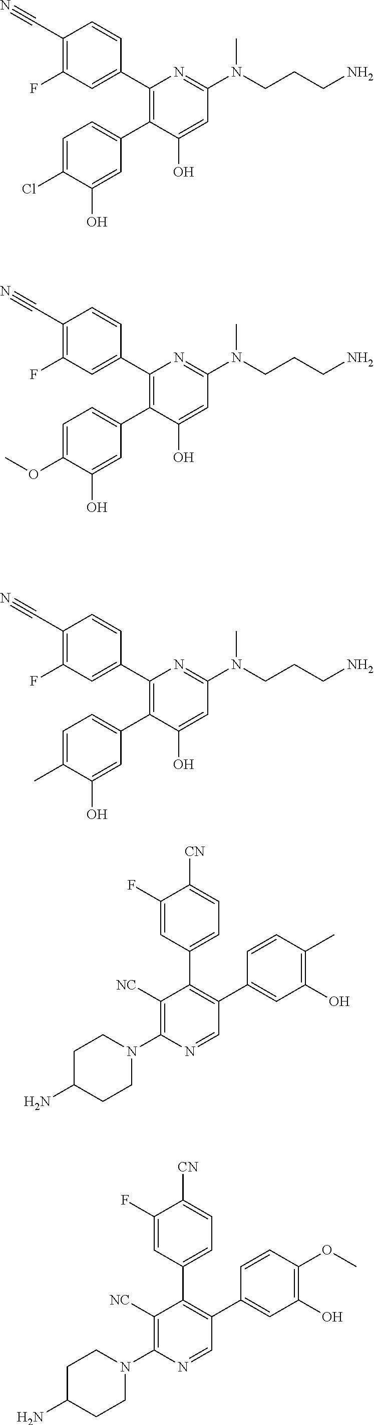 [SMILES: CN(CCCN)c1cc(O)c(-c2ccc(Cl)c(O)c2)c(-c2ccc(C#N)c(F)c2)n1 ; COc1ccc(-c2c(O)cc(N(C)CCCN)nc2-c2ccc(C#N)c(F)c2)cc1O ; Cc1ccc(-c2c(O)cc(N(C)CCCN)nc2-c2ccc(C#N)c(F)c2)cc1O ; Cc1ccc(-c2cnc(N3CCC(N)CC3)c(C#N)c2-c2ccc(C#N)c(F)c2)cc1O ; COc1ccc(-c2cnc(N3CCC(N)CC3)c(C#N)c2-c2ccc(C#N)c(F)c2)cc1O]

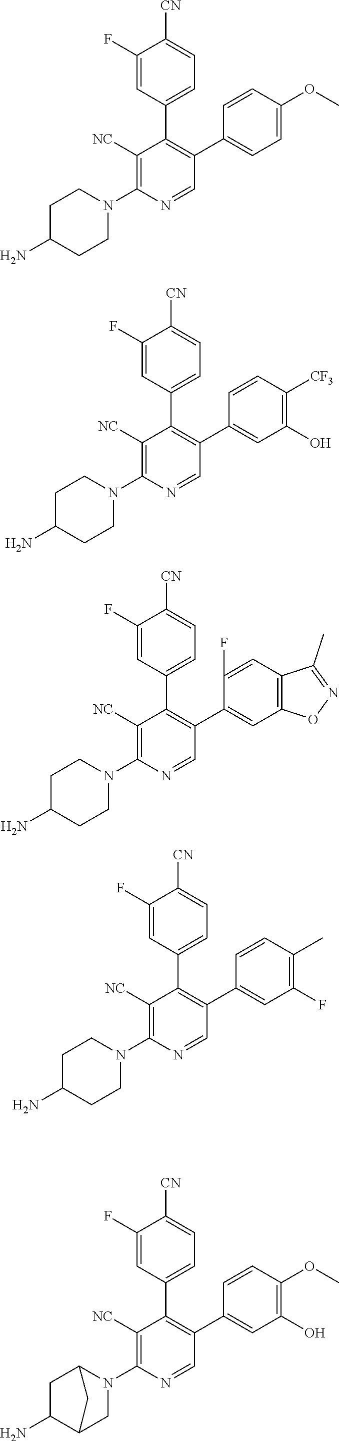 [SMILES: COc1ccc(-c2cnc(N3CCC(N)CC3)c(C#N)c2-c2ccc(C#N)c(F)c2)cc1 ; N#Cc1ccc(-c2c(-c3ccc(C(F)(F)F)c(O)c3)cnc(N3CCC(N)CC3)c2C#N)cc1F ; Cc1noc2cc(-c3cnc(N4CCC(N)CC4)c(C#N)c3-c3ccc(C#N)c(F)c3)c(F)cc12 ; Cc1ccc(-c2cnc(N3CCC(N)CC3)c(C#N)c2-c2ccc(C#N)c(F)c2)cc1F ; COc1ccc(-c2cnc(N3CC4CC3CC4N)c(C#N)c2-c2ccc(C#N)c(F)c2)cc1O]

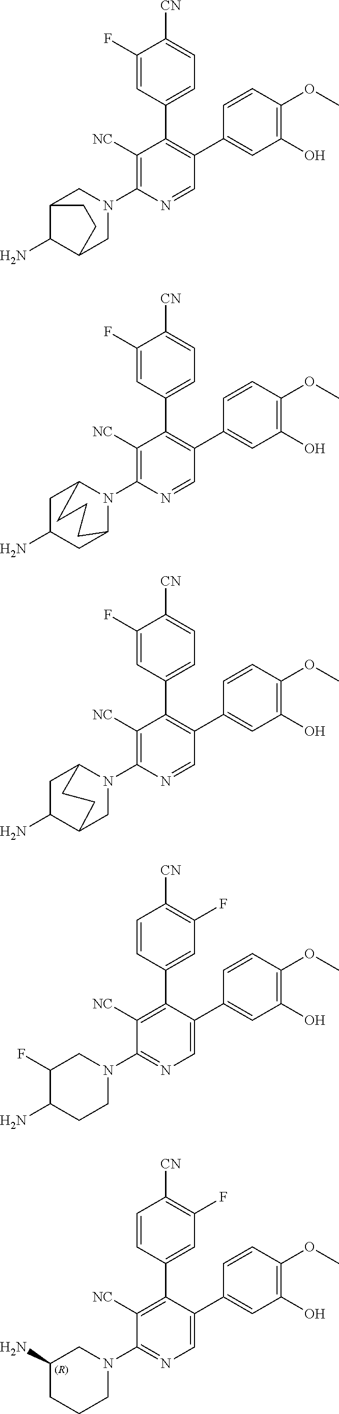

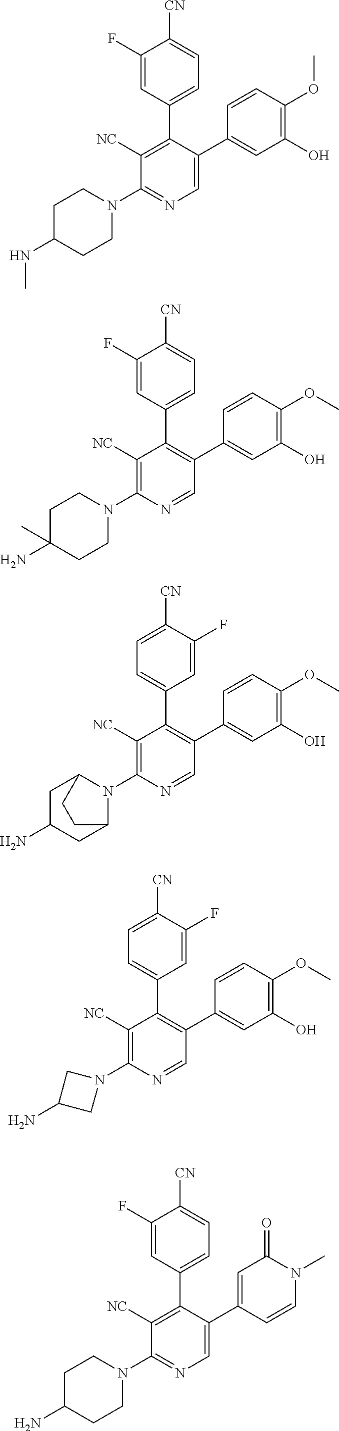 [SMILES: CNC1CCN(c2ncc(-c3ccc(OC)c(O)c3)c(-c3ccc(C#N)c(F)c3)c2C#N)CC1 ; COc1ccc(-c2cnc(N3CCC(C)(N)CC3)c(C#N)c2-c2ccc(C#N)c(F)c2)cc1O ; COc1ccc(-c2cnc(N3C4CCC3CC(N)C4)c(C#N)c2-c2ccc(C#N)c(F)c2)cc1O ; COc1ccc(-c2cnc(N3CC(N)C3)c(C#N)c2-c2ccc(C#N)c(F)c2)cc1O ; Cn1ccc(-c2cnc(N3CCC(N)CC3)c(C#N)c2-c2ccc(C#N)c(F)c2)cc1=O]

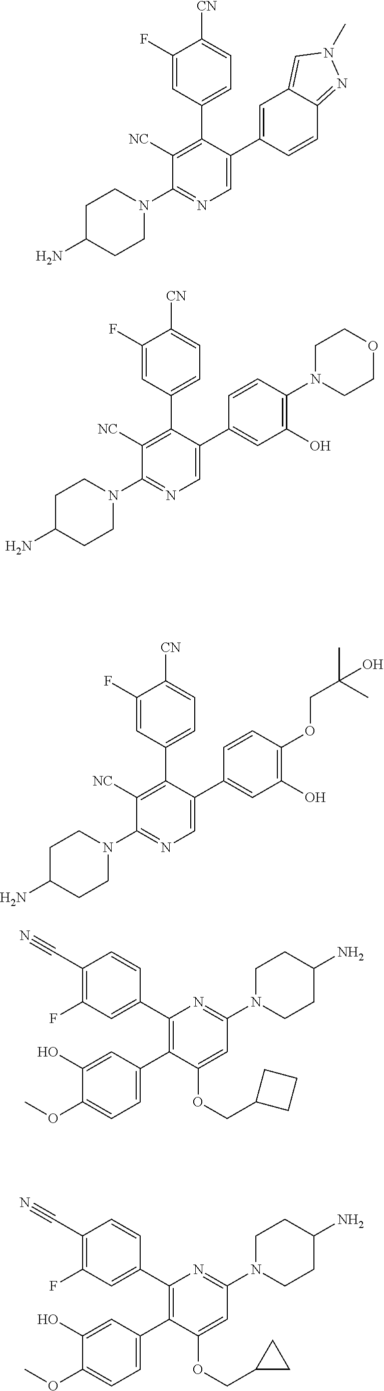

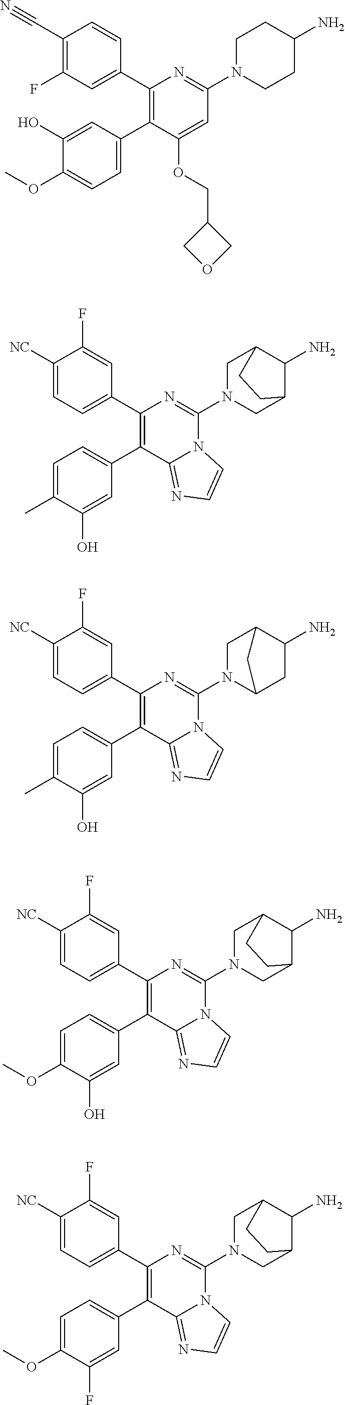 [SMILES: COc1ccc(-c2c(OCC3COC3)cc(N3CCC(N)CC3)nc2-c2ccc(C#N)c(F)c2)cc1O ; Cc1ccc(-c2c(-c3ccc(C#N)c(F)c3)nc(N3CC4CCC(C3)C4N)n3ccnc23)cc1O ; Cc1ccc(-c2c(-c3ccc(C#N)c(F)c3)nc(N3CC4CC3CC4N)n3ccnc23)cc1O ; COc1ccc(-c2c(-c3ccc(C#N)c(F)c3)nc(N3CC4CCC(C3)C4N)n3ccnc23)cc1O ; COc1ccc(-c2c(-c3ccc(C#N)c(F)c3)nc(N3CC4CCC(C3)C4N)n3ccnc23)cc1F]

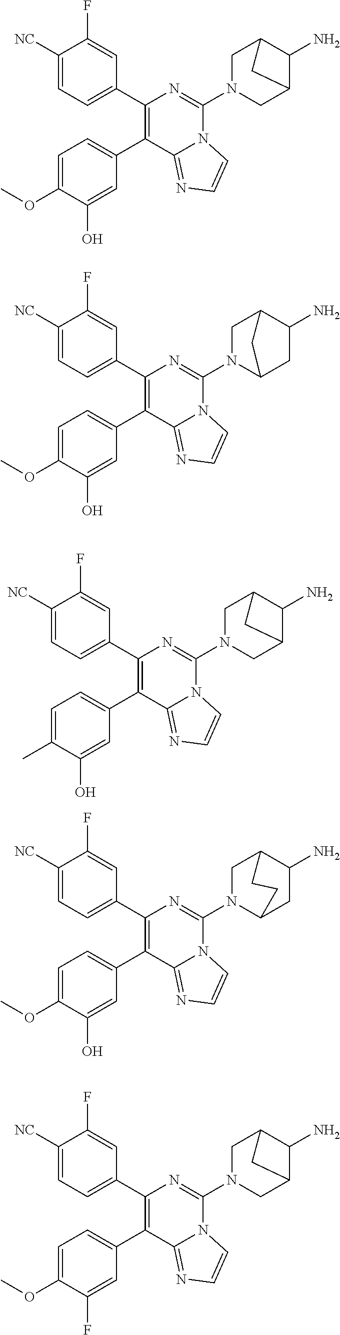

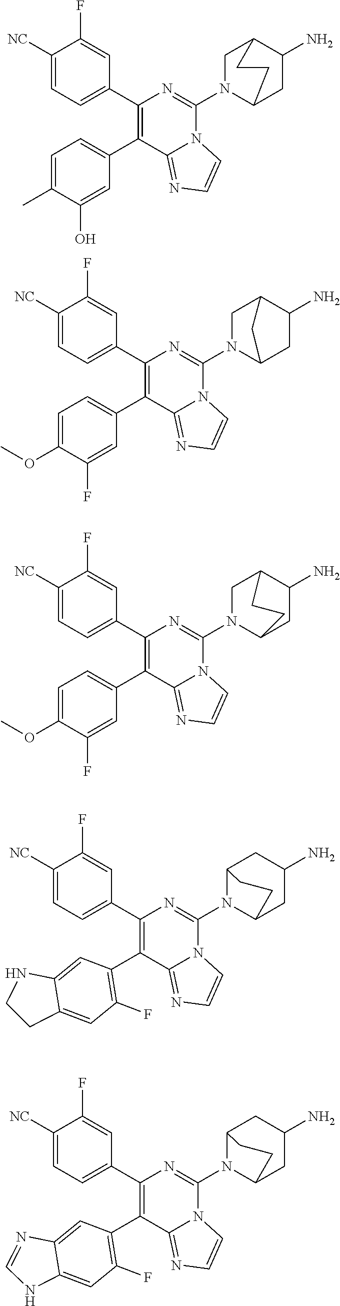

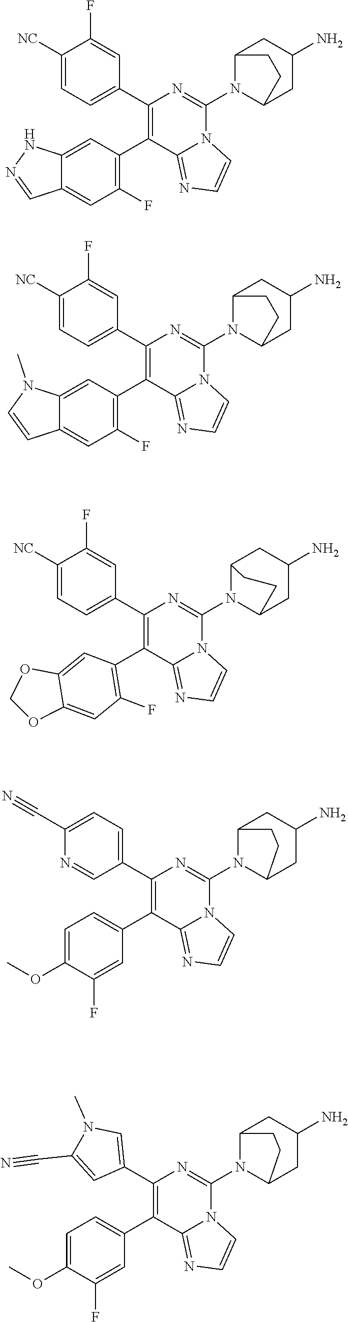

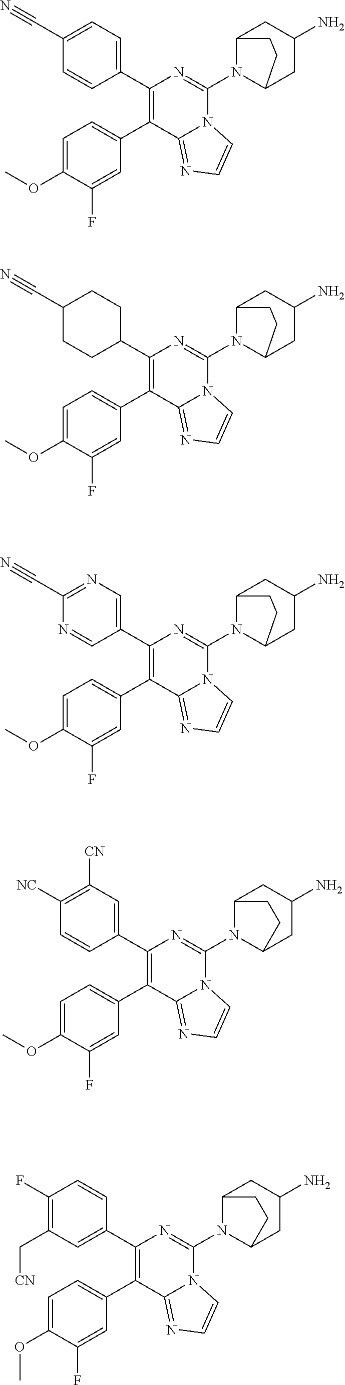 [SMILES: COc1ccc(-c2c(-c3ccc(C#N)cc3)nc(N3C4CCC3CC(N)C4)n3ccnc23)cc1F ; COc1ccc(-c2c(C3CCC(C#N)CC3)nc(N3C4CCC3CC(N)C4)n3ccnc23)cc1F ; COc1ccc(-c2c(-c3cnc(C#N)nc3)nc(N3C4CCC3CC(N)C4)n3ccnc23)cc1F ; COc1ccc(-c2c(-c3ccc(C#N)c(C#N)c3)nc(N3C4CCC3CC(N)C4)n3ccnc23)cc1F ; COc1ccc(-c2c(-c3ccc(F)c(CC#N)c3)nc(N3C4CCC3CC(N)C4)n3ccnc23)cc1F]

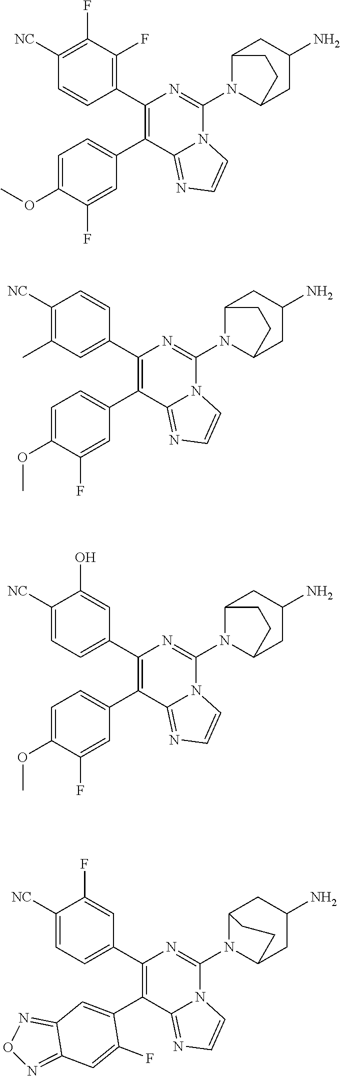 [SMILES: COc1ccc(-c2c(-c3ccc(C#N)c(F)c3F)nc(N3C4CCC3CC(N)C4)n3ccnc23)cc1F ; COc1ccc(-c2c(-c3ccc(C#N)c(C)c3)nc(N3C4CCC3CC(N)C4)n3ccnc23)cc1F ; COc1ccc(-c2c(-c3ccc(C#N)c(O)c3)nc(N3C4CCC3CC(N)C4)n3ccnc23)cc1F ; N#Cc1ccc(-c2nc(N3C4CCC3CC(N)C4)n3ccnc3c2-c2cc3nonc3cc2F)cc1F]

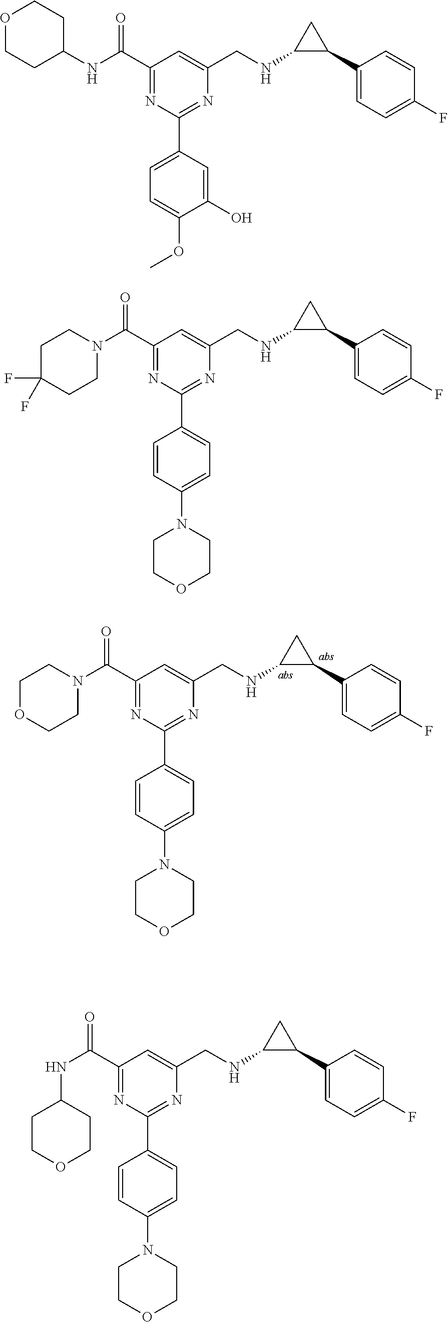

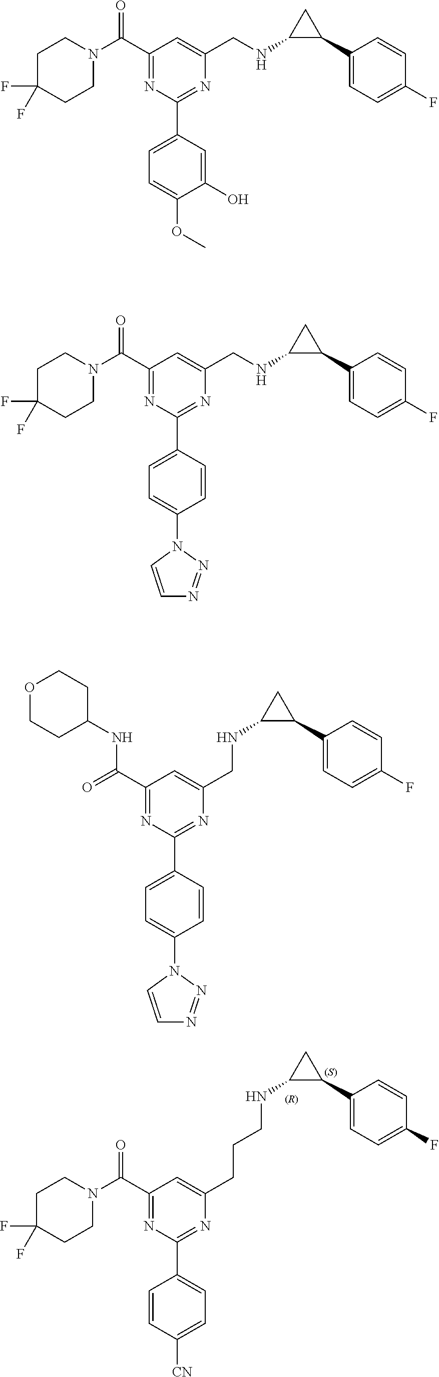

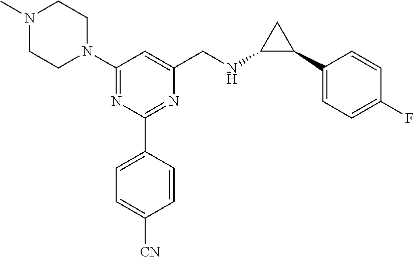 [SMILES: CN1CCN(c2cc(CN[C@@H]3C[C@H]3c3ccc(F)cc3)nc(-c3ccc(C#N)cc3)n2)CC1]

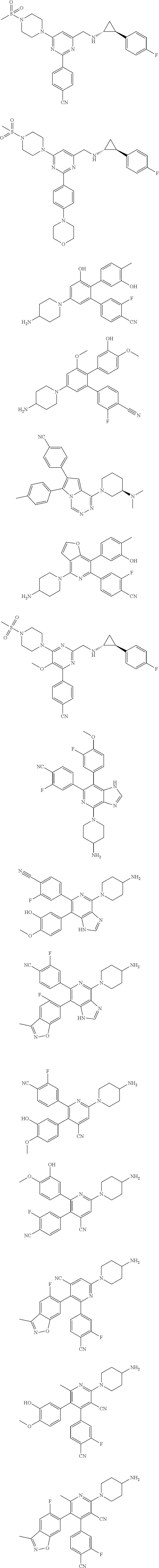

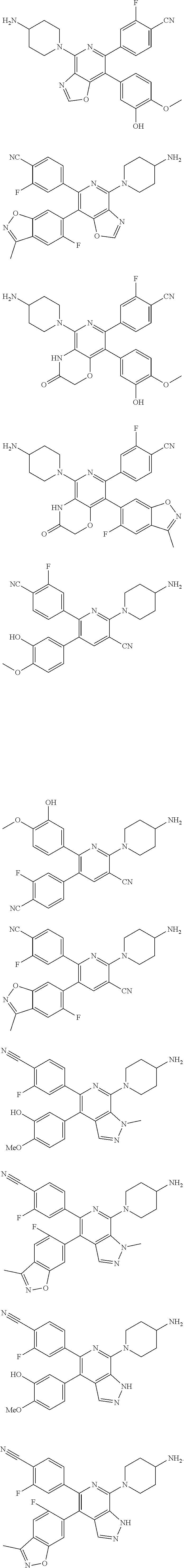

123. A pharmaceutical composition, the pharmaceutical composition comprising therapeutically effective amount of the compound according to claim 1 or the tautomer, the mesomer, the racemate, the enantiomer, the diastereoisomer, or the mixture thereof, or the deuterated isotope derivative, the pharmaceutically acceptable hydrate, the solvate, the salt or the cocrystal thereof, and a pharmaceutically acceptable carrier, diluent, adjuvant, vehicle or excipient; the composition may further comprise one or more other therapeutic agents.

124. A method for treating a disease related to LSD1, comprising administering the compound according to claim 1 or the tautomer, the mesomer, the racemate, the enantiomer, the diastereoisomer, or the mixture thereof, or the deuterated isotope derivative, the pharmaceutically acceptable hydrate, the solvate, the salt or the cocrystal thereof or the pharmaceutical composition comprising said compound to a subject in need.

125. The method according to claim 124, wherein the disease related to LSD1 is tumor.

126. The method according to claim 124, wherein the disease related to LSD1 is small cell lung cancer.

127. The compound according to claim 1, wherein ring A is selected from 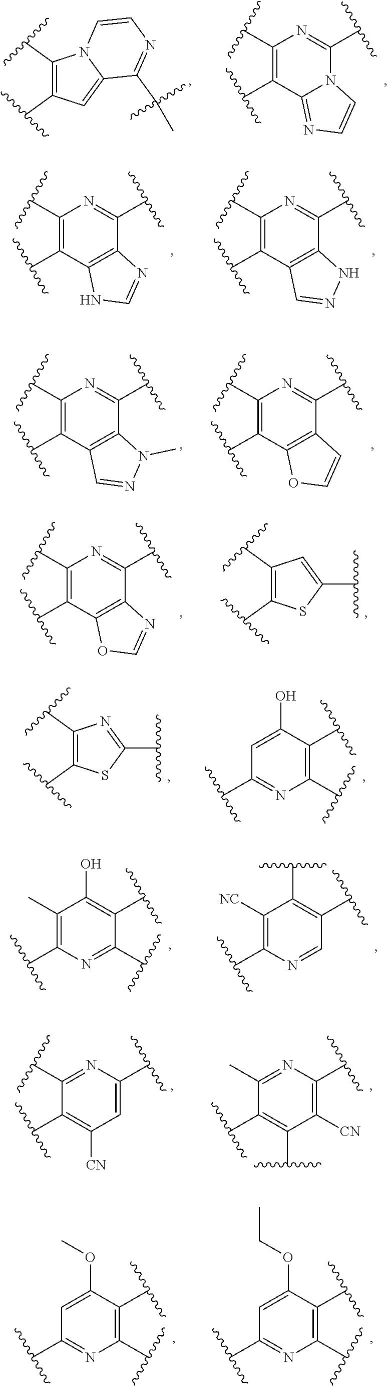 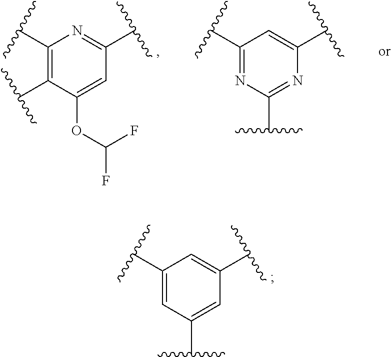 [SMILES: *c1cc(OC(F)F)c(*)c(*)n1 ; *c1cc(*)nc(*)n1 ; *c1cc(*)cc(*)c1]
ring B is selected from 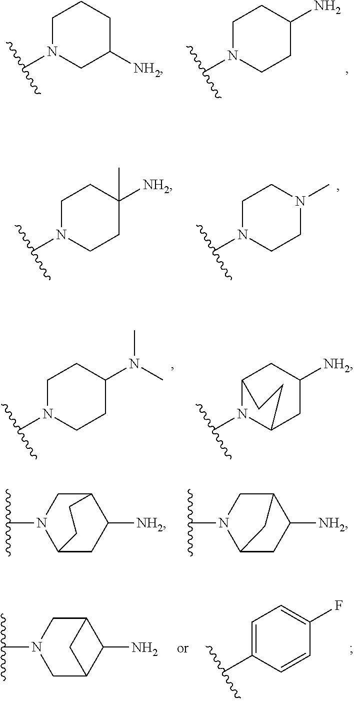
W is selected from a bond, —CH₂—, 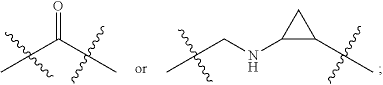
and
R₁ and/or R₂ is selected from: 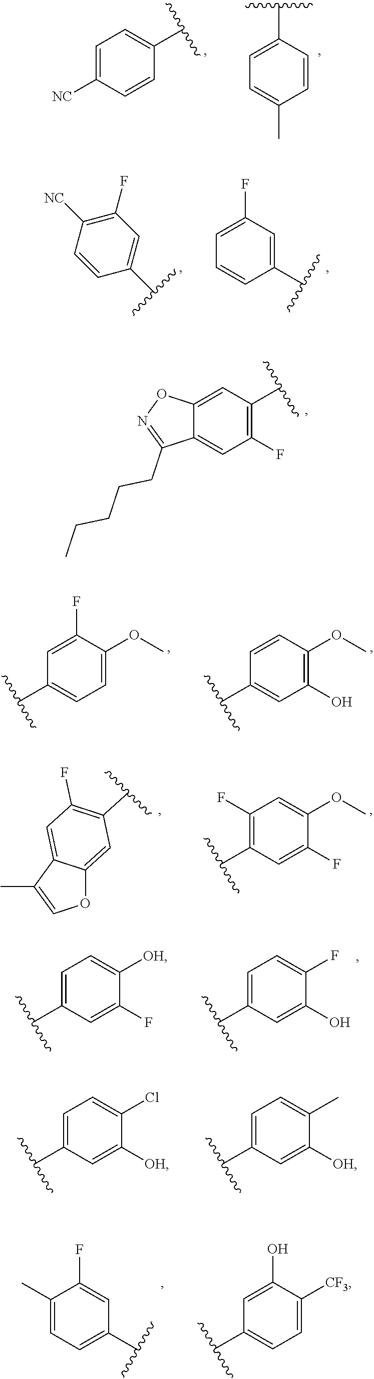 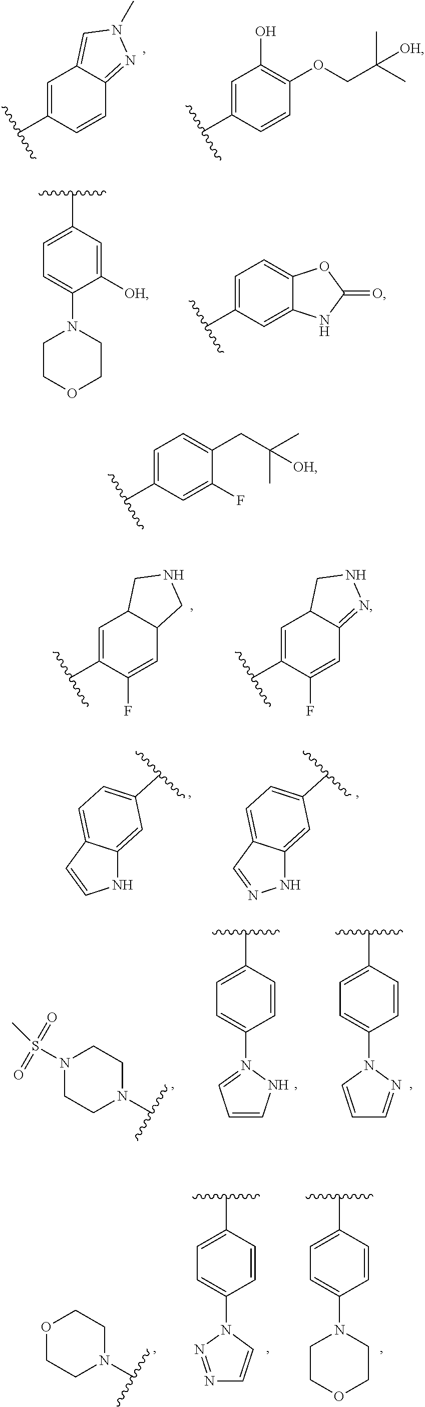 [chemical structure]
